(19) United States
(12) Patent Application Publication
(10) Pub. No.: US 2024/0352003 A1
(43) Pub. Date: Oct. 24, 2024

(54) HETEROCYCLIC COMPOUND, ORGANIC LIGHT EMITTING DEVICE COMPRISING SAME AND COMPOSITION FOR ORGANIC MATERIAL LAYER

(71) Applicant: LT MATERIALS CO., LTD., Yongin-si (KR)

(72) Inventors: Eui Jeong CHOI, Yongin-si (KR); Hyun Joo LEE, Yongin-si (KR); Geon Yu PARK, Yongin-si (KR); Young Seok NO, Yongin-si (KR); Dong Jun KIM, Yongin-si (KR); Dae Hyuk CHOI, Yongin-si (KR)

(73) Assignee: LT MATERIALS CO., LTD., Yongin-si, Gyeonggi-do (KR)

(21) Appl. No.: 18/214,786

(22) Filed: Jun. 27, 2023

(30) Foreign Application Priority Data
Apr. 7, 2023 (KR) 10-2023-0046235

(51) Int. Cl. C07D 403/08; C07D 487/04; H10K 50/11; H10K 50/15; H10K 50/17; H10K 50/18; H10K 85/60

(52) U.S. Cl. CPC C07D 403/08; C07D 487/04; H10K 50/11; H10K 50/15; H10K 50/171; H10K 50/181; H10K 85/6572; C07B 2200/05

(57) ABSTRACT

The present disclosure relates to a heterocyclic compound represented by Chemical Formula 1, an organic light emitting device including the same, and a composition for an organic material layer:

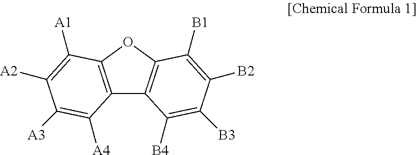

in Chemical Formula 1, each substituent has the same definition as described in the detailed description.

### CROSS-REFERENCE TO RELATED APPLICATION(S)

This application claims priority to Korean Patent Application No. 10-2023-0046235, filed on Apr. 7, 2023, the disclosure of which is incorporated herein by reference in its entirety.

### TECHNICAL FIELD

The present disclosure relates to a heterocyclic compound, an organic light emitting device including the same, and a composition for an organic material layer.

### DESCRIPTION OF THE RELATED ART

An organic light emitting device is one type of self-emissive display devices, and has advantages of having a wide viewing angle and a high response speed as well as having an excellent contrast.

The organic light emitting device has a structure of disposing an organic thin film between two electrodes. When a voltage is applied to the organic light emitting device having such a structure, electrons and holes injected from the two electrodes bind and pair in the organic thin film, and then light emits as these annihilate. The organic thin film may be formed in a single layer or a multilayer as necessary.

A material of the organic thin film may have a light emitting function as necessary. For example, as a material of the organic thin film, compounds each capable of forming a light emitting layer themselves alone may be used, or compounds each capable of performing a role of a host or a dopant of a host-dopant-based light emitting layer may also be used. In addition thereto, compounds capable of performing roles of hole injection, hole transport, electron blocking, hole blocking, electron transport, electron injection and the like may also be used as a material of the organic thin film.

Development of an organic thin film material has been continuously required for enhancing performance, lifetime or efficiency of an organic light emitting device.

### PRIOR ART DOCUMENTS

### Patent Documents

U.S. Pat. No. 4,356,429

### SUMMARY

The present disclosure is directed to providing a heterocyclic compound, an organic light emitting device including the same, and a composition for an organic material layer.

One embodiment of the present application provides a heterocyclic compound represented by the following Chemical Formula 1.

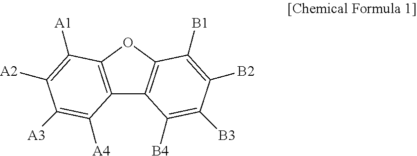

In Chemical Formula 1,
A1 to A4 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102 and —SiR101R102R103; and a group represented by the following Structural Formula 1, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
at least one of A1 to A4 is the group represented by the following Structural Formula 1,

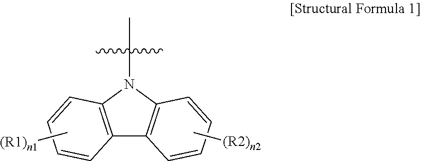

B1 to B4 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102 and —SiR101R102R103; and a group represented by the following Structural Formula 2, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a unsubstituted C2 to C60 heteroaryl group, and substituted or
at least one of B1 to B4 is the group represented by the following Structural Formula 2,

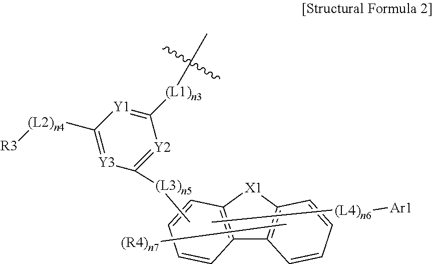

in Structural Formula 1,
R1 and R2 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102 and —SiR101R102R103, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group, and
n1 and n2 are the same as or different from each other, and each independently an integer of 0 to 4,
in Structural Formula 2,
X1 is O or S,
L1 to L4 are the same as or different from each other, and each independently a single bond; a substituted or unsubstituted C6 to C60 arylene group; or a substituted or unsubstituted C2 to C60 heteroarylene group,
Y1 to Y3 are the same as or different from each other and each independently CH or N, and at least one of Y1 to Y3 is N,
Ar1 is a substituted or unsubstituted C6 to C60 aryl group; or a substituted or unsubstituted C2 to C60 heteroaryl group,
R3 and R4 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102 and —SiR101R102R103, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
n3 to n6 are the same as or different from each other, and each independently an integer of 0 to 3, and
n7 is an integer of 0 to 6.

In addition, one embodiment of the present application provides an organic light emitting device including: a first electrode; a second electrode provided opposite to the first electrode; and one or more organic material layers provided between the first electrode and the second electrode, wherein one or more layers of the organic material layers include the heterocyclic compound represented by Chemical Formula 1.

In addition, one embodiment of the present application provides an organic light emitting device, wherein the organic material layer including the heterocyclic compound of Chemical Formula 1 further includes a heterocyclic compound represented by the following Chemical Formula 2 or Chemical Formula 3.

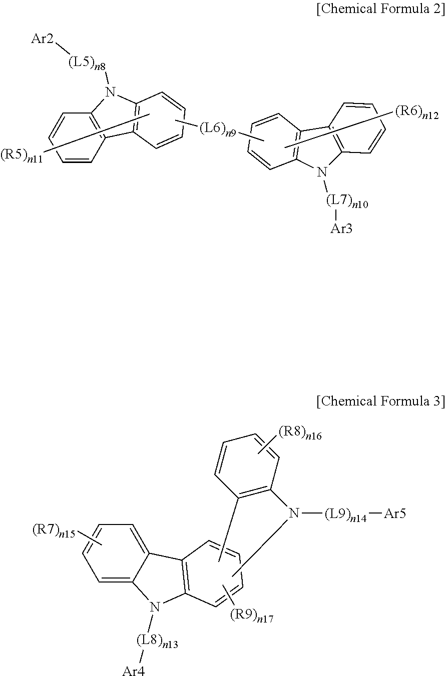

In Chemical Formulae 2 and 3,
L5 to L9 are the same as or different from each other, and each independently a single bond; a substituted or unsubstituted C6 to C60 arylene group; or a substituted or unsubstituted C2 to C60 heteroarylene group,
Ar2 to Ar5 are the same as or different from each other, and each independently a substituted or unsubstituted C6 to C60 aryl group; or a substituted or unsubstituted C2 to C60 heteroaryl group,
R5 to R9 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102 and —SiR101R102R103, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
n8 to n10, n13 and n14 are the same as or different from each other, and each independently an integer of 0 to 3,
n11 and n12 are an integer of 0 to 7,
n15 and n16 are an integer of 0 to 4, and
n17 is an integer of 0 to 2.

In addition, another embodiment of the present application provides a composition for an organic material layer, the composition including: the heterocyclic compound represented by Chemical Formula 1, and the heterocyclic compound represented by Chemical Formula 2 or 3.

The heterocyclic compound according to one embodiment can be used as an organic material layer material of an organic light emitting device. The compound is capable of performing roles of a hole injection layer material, an electron blocking layer material, a hole transport layer material, a light emitting layer material, an electron transport layer material, a hole blocking layer material, an electron injection layer material and the like in an organic light emitting device. Particularly, the compound can be used as a light emitting layer material of an organic light emitting device.

The heterocyclic compound can be used as a light emitting material either alone or as a mixture with a P-type host, and may be used as a host material or a dopant material of a light emitting layer.

Particularly, in the heterocyclic compound represented by Chemical Formula 1, the dibenzofuran parent structure

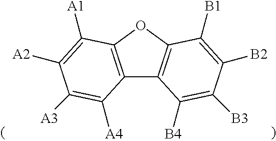

forms a resonance structure, and accordingly, the No. 2 and No. 4 carbon sites of the benzene ring included in the parent structure are negatively charged, and the No. 1 and No. 3 carbon sites become relatively electron deficient. Herein, when bonding an electron transport moiety to the No. 1 and No. 3 carbon sites that are relatively electron deficient and bonding a hole transport moiety to the No. 2 and No. 4 carbon sites that are relatively electron abundant, the transport speed of electrons in the molecule becomes faster. Accordingly, when a triazine substituent is introduced to the relatively electron-deficient No. 1 and No. 3 carbon sites of the benzene ring in the dibenzofuran parent structure as in the heterocyclic compound represented by Chemical Formula 1 according to the present disclosure, the role of the triazine substituent is strengthened compared to when a triazine substituent is introduced to the No. 2 and No. 4 carbon sites of the benzene ring.

In addition, the heterocyclic compound represented by Chemical Formula 1 according to the present disclosure further includes a dibenzofuran or dibenzothiophene substituent in the triazine substituent linked to the dibenzofuran parent structure, and further includes an aryl group or a heteroaryl group in the dibenzofuran or dibenzothiophene substituent, and accordingly, electron transport can be strengthened through expanding aromaticity of the LUMO (lowest unoccupied molecular orbital) level and the resonance structure, and when the heterocyclic compound represented by Chemical Formula 1 is used in an organic light emitting device together with the heterocyclic compound according to Chemical Formula 2 or 3 according to the present disclosure, there is an effect that intermolecular electron transport occurs more smoothly.

Accordingly, by using the compound represented by Chemical Formula 1 in an organic material layer, it is possible to lower a driving voltage of an organic light emitting device, enhance light emission efficiency, and enhance lifetime properties of an organic light emitting device due to thermal stability of the compound.

### BRIEF DESCRIPTION OF THE DRAWINGS

FIGS. 1 to 3 are diagrams each schematically illustrating a lamination structure of an organic light emitting device according to one embodiment of the present disclosure.

### DESCRIPTION OF SPECIFIC EMBODIMENTS

Hereinafter, the present disclosure will be described in more detail.

In the present specification, a term “substitution” means that a hydrogen atom bonding to a carbon atom of a compound is changed to another substituent, and the position of substitution is not limited as long as it is a position at which the hydrogen atom is substituted, that is, a position at which a substituent is capable of substituting, and when two or more substituents substitute, the two or more substituents may be the same as or different from each other.

In the present specification, a term “substituted or unsubstituted” means being substituted with one or more substituents selected from the group consisting of deuterium; halogen; a cyano group; a C1 to C60 linear or branched alkyl group; a C2 to C60 linear or branched alkenyl group; a C2 to C60 linear or branched alkynyl group; a C3 to C60 monocyclic or polycyclic cycloalkyl group; a C2 to C60 monocyclic or polycyclic heterocycloalkyl group; a C6 to C60 monocyclic or polycyclic aryl group; a C2 to C60 monocyclic or polycyclic heteroaryl group; —SiRR′R″; —P(═O)RR′; a C1 to C20 alkylamine group; a C6 to C60 monocyclic or polycyclic arylamine group; and a C2 to C60 monocyclic or polycyclic heteroarylamine group or being unsubstituted, or being substituted with a substituent in which two or more substituents selected from among the substituents exemplified above are linked or being unsubstituted.

In the present specification, “the number of protons” means the number of substituents that a specific compound may have, and specifically, the number of protons may mean the number of hydrogens.

For example, unsubstituted benzene may be expressed to have the number of protons of 5, an unsubstituted naphthyl group may be expressed to have the number of protons of 7, a naphthyl group substituted with a phenyl group may be expressed to have the number of protons of 6, and an unsubstituted biphenyl group may be expressed to have the number of protons of 9.

In the present specification, the halogen may be fluorine, chlorine, bromine or iodine.

In the present specification, the alkyl group includes a linear or branched form having 1 to 60 carbon atoms, and may be further substituted with other substituents. The number of carbon atoms of the alkyl group may be from 1 to 60, specifically from 1 to 40 and more specifically from 1 to 20. Specific examples thereof may include a methyl group, an ethyl group, a propyl group, an n-propyl group, an isopropyl group, an n-butyl group, an isobutyl group, a tert-butyl group, a sec-butyl group, a 1-methyl-butyl group, a 1-ethyl-butyl group, an n-pentyl group, an isopentyl group, a neopentyl group, a tert-pentyl group, an n-hexyl group, a 1-methylpentyl group, a 4-methyl-2-pentyl group, a 3,3-dimethylbutyl group, a 2-ethylbutyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group and the like, but are not limited thereto.

In the present specification, the alkenyl group includes a linear or branched form having 2 to 60 carbon atoms, and may be further substituted with other substituents. The number of carbon atoms of the alkenyl group may be from 2 to 60, specifically from 2 to 40 and more specifically from 2 to 20. Specific examples thereof may include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl) vinyl-1-yl group, a 2,2-bis(diphenyl-1-yl) vinyl-1-yl group, a stilbenyl group, a styrenyl group and the like, but are not limited thereto.

In the present specification, the alkynyl group includes a linear or branched form having 2 to 60 carbon atoms, and may be further substituted with other substituents. The number of carbon atoms of the alkynyl group may be from 2 to 60, specifically from 2 to 40 and more specifically from 2 to 20.

In the present specification, the alkoxy group may be linear, branched or cyclic. The number of carbon atoms of the alkoxy group is not particularly limited, but is preferably from 1 to 20. Specific examples thereof may include a methoxy group, an ethoxy group, an n-propoxy group, an isopropoxy group, an n-butoxy group, an isobutoxy group, a tert-butoxy group, a sec-butoxy group, an n-pentyloxy group, a neopentyloxy group, an isopentyloxy group, an n-hexyloxy group, a 3,3-dimethylbutyloxy group, a 2-ethylbutyloxy group, an n-octyloxy group, an n-nonyloxy group, an n-decyloxy group, a benzyloxy group, a p-methylbenzyloxy group and the like, but are not limited thereto.

In the present specification, the cycloalkyl group includes a monocyclic or polycyclic group having 3 to 60 carbon atoms, and may be further substituted with other substituents. Herein, the polycyclic group means a group in which the cycloalkyl group is directly linked to or fused with another cyclic group. Herein, the another cyclic group may be a cycloalkyl group, but may also be different types of cyclic groups such as a heterocycloalkyl group, an aryl group and a heteroaryl group. The number of carbon atoms of the cycloalkyl group may be from 3 to 60, specifically from 3 to 40 and more specifically from 5 to 20. Specific examples thereof may include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group and the like, but are not limited thereto.

In the present specification, the heterocycloalkyl group includes O, S, Se, N or Si as a heteroatom, includes a monocyclic or polycyclic group having 2 to 60 carbon atoms, and may be further substituted with other substituents. Herein, the polycyclic group means a group in which the heterocycloalkyl group is directly linked to or fused with another cyclic group. Herein, the another cyclic group may be a heterocycloalkyl group, but may also be different types of cyclic groups such as a cycloalkyl group, an aryl group and a heteroaryl group. The number of carbon atoms of the heterocycloalkyl group may be from 2 to 60, specifically from 2 to 40 and more specifically from 3 to 20.

In the present specification, the aryl group includes a monocyclic or polycyclic group having 6 to 60 carbon atoms, and may be further substituted with other substituents. Herein, the polycyclic group means a group in which the aryl group is directly linked to or fused with another cyclic group. Herein, the another cyclic group may be an aryl group, but may also be different types of cyclic groups such as a cycloalkyl group, a heterocycloalkyl group and a heteroaryl group. The aryl group may include a spiro group. The number of carbon atoms of the aryl group may be from 6 to 60, specifically from 6 to 40 and more specifically from 6 to 25. Specific examples of the aryl group may include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused ring group thereof, and the like, but are not limited thereto.

In the present specification, the phosphine oxide group is represented by —P(═O)R101R102, and R101 and R102 are the same as or different from each other and may be each independently a substituent formed with at least one of hydrogen; deuterium; a halogen group; an alkyl group; an alkenyl group; an alkoxy group; a cycloalkyl group; an aryl group; and a heterocyclic group. Specifically, the phosphine oxide group may be substituted with an aryl group, and as the aryl group, the examples described above may be applied. Examples of the phosphine oxide group may include a diphenylphosphine oxide group, a dinaphthylphosphine oxide group and the like, but are not limited thereto.

In the present specification, the silyl group is a substituent including Si and having the Si atom directly linked as a radical, and is represented by —SiR101R102R103. R101 to R103 are the same as or different from each other, and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present specification, the fluorenyl group may be substituted, and adjacent substituents may bond to each other to form a ring.

When the fluorenyl group is substituted, the following structural formulae and the like may be included, however, the structure is not limited thereto.

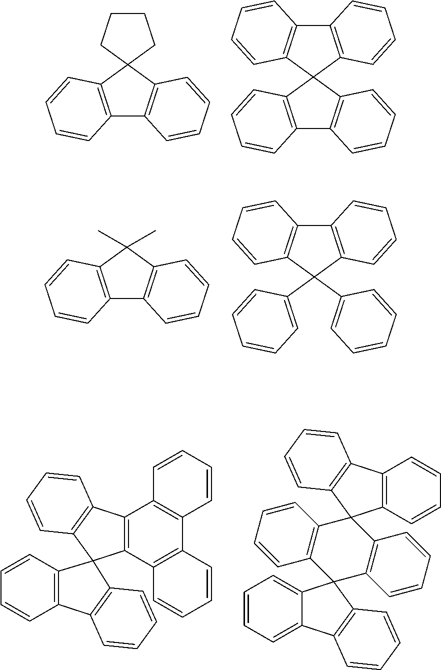

In the present specification, the spiro group is a group including a spiro structure, and may have 15 to 60 carbon atoms. For example, the spiro group may include a structure in which a 2,3-dihydro-1H-indene group or a cyclohexane group spiro bonds to a fluorenyl group. Specifically, the spiro group may include any one of groups of the following structural formulae.

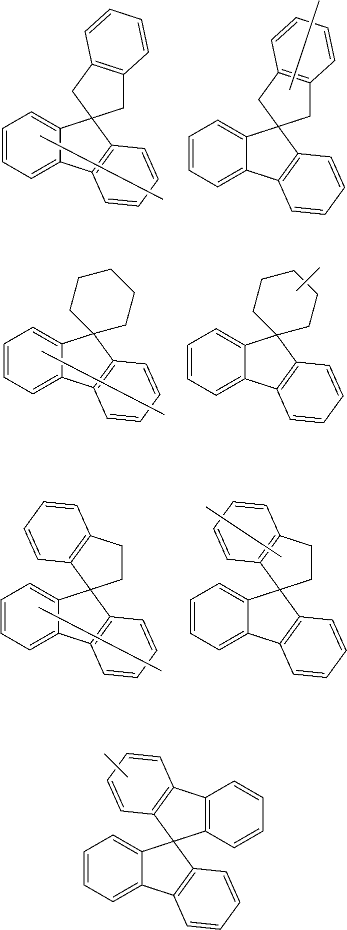

In the present specification, the heteroaryl group includes S, O, Se, N or Si as a heteroatom, includes a monocyclic or polycyclic group having 2 to 60 carbon atoms, and may be further substituted with other substituents. Herein, the polycyclic group means a group in which the heteroaryl group is directly linked to or fused with another cyclic group. Herein, the another cyclic group may be a heteroaryl group, but may also be different types of cyclic groups such as a cycloalkyl group, a heterocycloalkyl group and an aryl group. The number of carbon atoms of the heteroaryl group may be from 2 to 60, specifically from 2 to 40 and more specifically from 3 to 25. Specific examples of the heteroaryl group may include a pyridyl group, a pyrrolyl group, a pyrimidyl group, a pyridazinyl group, a furanyl group, a thiophenyl group, an imidazolyl group, a pyrazolyl group, an oxazolyl group, an isoxazolyl group, thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a quinozolinyl group, a naphthyridyl group, an acridinyl group, a phenanthridinyl group, an imidazopyridinyl group, a diazanaphthalenyl group, a triazaindenyl group, a 2-indolyl group, an indolizinyl group, a benzothiazolyl group, a benzoxazolyl group, a benzimidazolyl group, a benzothiophenyl group, a benzofuranyl group, a dibenzothiophenyl group, a dibenzofuranyl group, a carbazolyl a benzocarbazolyl group, a dibenzocarbazolyl group, a phenazinyl group, a dibenzosilole group, a spirobi (dibenzosilole) group, a dihydrophenazinyl group, a phenoxazinyl group, a phenanthridyl group, a thienyl group, an indolo[2,3-a]carbazolyl group, an indolo[2,3-b]carbazolyl group, an indolinyl group, a 10,11-dihydro-dibenzo[b,f]azepinyl group, a 9,10-dihydroacridinyl group, a phenanthrazinyl group, a phenothiazinyl group, a phthalazinyl group, a naphthylidinyl group, a phenanthrolinyl group, a benzo[c][1,2,5]thiadiazolyl group, a 5,10-dihydrodibenzo[b,e][1,4]azasilinyl group, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group and the like, but are not limited thereto.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and the number of carbon atoms is not particularly limited, but preferably from 1 to 30. Specific examples of the amine group may include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like, but are not limited thereto.

In the present specification, the arylene group means the aryl group having two bonding sites, that is, a divalent group. The descriptions on the aryl group provided above may be applied thereto except for those that are each a divalent group. In addition, the heteroarylene group means the heteroaryl group having two bonding sites, that is, a divalent group. The descriptions on the heteroaryl group provided above may be applied thereto except for those that are each a divalent group.

In the present specification, an “adjacent” group may mean a substituent substituting an atom directly linked to an atom substituted by the corresponding substituent, a substituent sterically most closely positioned to the corresponding substituent, or another substituent substituting an atom substituted by the corresponding substituent. For example, two substituents substituting at ortho positions in a benzene ring, and two substituents substituting at the same carbon in an aliphatic ring may be interpreted as groups “adjacent” to each other.

In the present disclosure, a “case of a substituent being not indicated in a chemical formula or compound structure” means that a hydrogen atom bonds to a carbon atom. However, since deuterium (2H) is an isotope of hydrogen, some hydrogen atoms may be deuterium.

In one embodiment of the present disclosure, a “case of a substituent being not indicated in a chemical formula or compound structure” may mean that positions to which substituents may come are all hydrogen or deuterium. In other words, since deuterium is an isotope of hydrogen, some hydrogen atoms may be deuterium that is an isotope, and herein, a content of the deuterium may be from 0% to 100%.

In one embodiment of the present disclosure, in a “case of a substituent being not indicated in a chemical formula or compound structure”, hydrogen and deuterium may be used interchangeably in compounds when deuterium is not explicitly excluded such as “a deuterium content being 0%”, “a hydrogen content being 100%” or “substituents being all hydrogen”.

In one embodiment of the present disclosure, deuterium is one of isotopes of hydrogen, is an element having deuteron formed with one proton and one neutron as a nucleus, and may be expressed as hydrogen-2, and the elemental symbol thereof may also be written as D or ²H.

In one embodiment of the present disclosure, an isotope means an atom with the same atomic number (Z) but with a different mass number (A), and may also be interpreted as an element with the same number of protons but with a different number of neutrons.

In one embodiment of the present disclosure, a content T % of a specific substituent may be defined as T2/T1×100=T % when the total number of substituents that a basic compound may have is defined as T1, and the number of specific substituents among these is defined as T2.

In other words, in one example, having a deuterium content of 20% in a phenyl group represented by

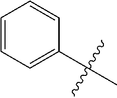 [SMILES: *c1ccccc1]

may mean that the total number of substituents that the phenyl group may have is 5 (T1 in the formula), and the number of deuterium among these is 1 (T2 in the formula). In other words, having a deuterium content of 20% in a phenyl group may be represented by the following structural formulae.

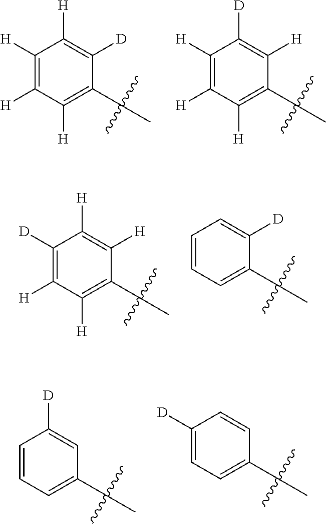 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

In addition, in one embodiment of the present disclosure, “a phenyl group having a deuterium content of 0%” may mean a phenyl group that does not include a deuterium atom, that is, a phenyl group that has 5 hydrogen atoms.

In the present disclosure, the C6 to C60 aromatic hydrocarbon ring means a compound including an aromatic ring formed with C6 to C60 carbons and hydrogens. Examples thereof may include benzene, biphenyl, terphenyl, triphenylene, naphthalene, anthracene, phenalene, phenanthrene, fluorene, pyrene, chrysene, perylene, azulene and the like, but are not limited thereto, and include all aromatic hydrocarbon ring compounds known in the art and satisfying the above-mentioned number of carbon atoms.

One embodiment of the present application provides a heterocyclic compound represented by the following Chemical Formula 1.

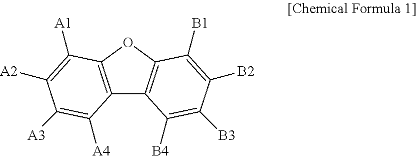

In Chemical Formula 1,
A1 to A4 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102 and —SiR101R102R103; and a group represented by the following Structural Formula 1, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
at least one of A1 to A4 is the group represented by the following Structural Formula 1,

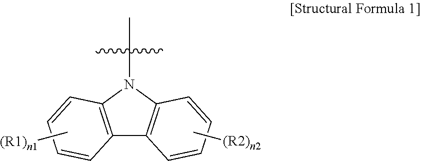

B1 to B4 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102 and —SiR101R102R103; and a group represented by the following Structural Formula 2, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group, and
at least one of B1 to B4 is the group represented by the following Structural Formula 2,

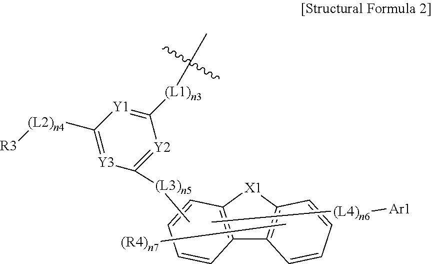

in Structural Formula 1,
R1 and R2 are the same as or different from each other, and each independently selected from the group consisting of hydrogen; deuterium; halogen; a cyano group; a substituted or unsubstituted C1 to C60 alkyl group; a substituted or unsubstituted to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102 and —SiR101R102R103, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group, and
n1 and n2 are the same as or different from each other, and each independently an integer of 0 to 4,
in Structural Formula 2,
X1 is O or S,
L1 to L4 are the same as or different from each other, and each independently a single bond; a substituted or unsubstituted C6 to C60 arylene group; or a substituted or unsubstituted C2 to C60 heteroarylene group,
Y1 to Y3 are the same as or different from each other and each independently CH or N, and at least one of Y1 to Y3 is N,
Ar1 is a substituted or unsubstituted C6 to C60 aryl group; or a substituted or unsubstituted C2 to C60 heteroaryl group,
R3 and R4 are the same as or different from each other, and each independently selected from the group consisting of hydrogen; deuterium; halogen; a cyano group; a substituted or unsubstituted C1 or to C60 alkyl group; a substituted unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102 and —SiR101R102R103, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
n3 to n6 are the same as or different from each other, and each independently an integer of 0 to 3, and
n7 is an integer of 0 to 6.

In one embodiment of the present application, A1 to A4 of Chemical Formula 1 are the same as or different from each other, and each independently selected from the group consisting of hydrogen; deuterium; halogen; a cyano group; a substituted or unsubstituted C1 to C40 alkyl group; a substituted or unsubstituted C2 to C40 alkenyl group; a substituted or unsubstituted C2 to C40 alkynyl group; a substituted or unsubstituted C1 to C40 alkoxy group; a substituted or unsubstituted C3 to C40 cycloalkyl group; a substituted or unsubstituted C2 to C40 heterocycloalkyl group; a substituted or unsubstituted C6 to C40 aryl group; a substituted or unsubstituted C2 to C40 heteroaryl group; —P(═O)R101R102 and —SiR101R102R103; and the group represented by Structural Formula 1, or two or more groups adjacent to each other bond to each other to form a substituted or unsubstituted C6 to C40 aromatic hydrocarbon ring or a substituted or unsubstituted C2 to C40 heteroring, R101, R102 and R103 are the same as or different from each other and each independently a substituted or unsubstituted C1 to C40 alkyl group; a substituted or unsubstituted C6 to C40 aryl group; or a substituted or unsubstituted C2 to C40 heteroaryl group, and at least one of A1 to A4 may be the group represented by Structural Formula 1.

In one embodiment of the present application, A1 to A4 of Chemical Formula 1 are the same as or different from each other, and each independently selected from the group consisting of hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R101R102 and —SiR101R102R103; and the group represented by Structural Formula 1, or two or more groups adjacent to each other bond to each other to form a substituted or unsubstituted C6 to C30 aromatic hydrocarbon ring or a substituted or unsubstituted C2 to C30 heteroring, R101, R102 and R103 are the same as or different from each other and each independently a substituted or unsubstituted C1 to C30 alkyl group; a substituted or unsubstituted C6 to C30 aryl group; or a substituted or unsubstituted C2 to C30 heteroaryl group, and at least one of A1 to A4 may be the group represented by Structural Formula 1.

In one embodiment of the present application, A1 to A4 of Chemical Formula 1 are the same as or different from each other, and each independently selected from the group consisting of hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; and the group represented by Structural Formula 1, or two or more groups adjacent to each other bond to each other to form a substituted or unsubstituted C6 to C20 aromatic hydrocarbon ring or a substituted or unsubstituted C2 to C20 heteroring, and at least one of A1 to A4 may be the group represented by Structural Formula 1.

In one embodiment of the present application, A1 to A4 of Chemical Formula 1 are the same as or different from each other, and each independently selected from the group consisting of hydrogen; deuterium; halogen; a cyano group; a substituted or unsubstituted C1 to C10 alkyl group; a substituted or unsubstituted C2 to C10 alkenyl group; a substituted or to C10 alkynyl group; a substituted or unsubstituted C2 unsubstituted C1 to C10 alkoxy group; a substituted or unsubstituted C3 to C10 cycloalkyl group; a substituted or unsubstituted C2 to C10 heterocycloalkyl group; a substituted or unsubstituted C6 to C10 aryl group; a substituted or unsubstituted C2 to C10 heteroaryl group; and the group represented by Structural Formula 1, or two or more groups adjacent to each other bond to each other to form a substituted or unsubstituted C6 to C10 aromatic hydrocarbon ring or a substituted or unsubstituted C2 to C10 heteroring, and at least one of A1 to A4 may be the group represented by Structural Formula 1.

In one embodiment of the present application, A1 to A4 of Chemical Formula 1 are the same as or different from each other, and may be each independently hydrogen; deuterium; or the group represented by Structural Formula 1.

In one embodiment of the present application, A1 of Chemical Formula 1 is the group represented by Structural Formula 1, and A2 to A4 are the same as or different from each other and may be each independently hydrogen; or deuterium.

In one embodiment of the present application, A2 of Chemical Formula 1 is the group represented by Structural Formula 1, and A1, A3 and A4 are the same as or different from each other and may be each independently hydrogen; or deuterium.

In one embodiment of the present application, A3 of Chemical Formula 1 is the group represented by Structural Formula 1, and A1, A2 and A4 are the same as or different from each other and may be each independently hydrogen; or deuterium.

In one embodiment of the present application, A4 of Chemical Formula 1 is the group represented by Structural Formula 1, and A1 to A3 are the same as or different from each other and may be each independently hydrogen; or deuterium.

In one embodiment of the present application, B1 to B4 of Chemical Formula 1 are the same as or different from each other, and each independently selected from the group consisting of hydrogen; deuterium; halogen; a cyano group; a substituted or unsubstituted C1 to C40 alkyl group; a substituted or unsubstituted C2 to C40 alkenyl group; a substituted or unsubstituted C2 to C40 alkynyl group; a substituted or unsubstituted C1 to C40 alkoxy group; a substituted or unsubstituted C3 to C40 cycloalkyl group; a substituted or unsubstituted C2 to C40 heterocycloalkyl group; a substituted or unsubstituted C6 to C40 aryl group; a substituted or unsubstituted C2 to C40 heteroaryl group; —P(═O)R101R102 and —SiR101R102R103; and the group represented by Structural Formula 2, or two or more groups adjacent to each other bond to each other to form a substituted or unsubstituted C6 to C40 aromatic hydrocarbon ring or a substituted or unsubstituted C2 to C40 heteroring, R101, R102 and R103 are the same as or different from each other and each independently a substituted or unsubstituted C1 to C40 alkyl group; a substituted or unsubstituted C6 to C40 aryl group; or a substituted or unsubstituted C2 to C40 heteroaryl group, and at least one of B1 to B4 may be the group represented by Structural Formula 2.

In one embodiment of the present application, B1 to B4 of Chemical Formula 1 are the same as or different from each other, and each independently selected from the group consisting of hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R101R102 and —SiR101R102R103; and the group represented by Structural Formula 2, or two or more groups adjacent to each other bond to each other to form a substituted or unsubstituted C6 to C30 aromatic hydrocarbon ring or a substituted or unsubstituted C2 to C30 heteroring, R101, R102 and R103 are the same as or different from each other and each independently a substituted or unsubstituted C1 to C30 alkyl group; a substituted or unsubstituted C6 to C30 aryl group; or a substituted or unsubstituted C2 to C30 heteroaryl group, and at least one of A1 to A4 may be the group represented by Structural Formula 2.

In one embodiment of the present application, B1 to B4 of Chemical Formula 1 are the same as or different from each other, and each independently selected from the group consisting of hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; and the group represented by Structural Formula 2, or two or more groups adjacent to each other bond to each other to form a substituted or unsubstituted C6 to C20 aromatic hydrocarbon ring or a substituted or unsubstituted C2 to C20 heteroring, and at least one of B1 to B4 may be the group represented by Structural Formula 2.

In one embodiment of the present application, B1 to B4 of Chemical Formula 1 are the same as or different from each other, and each independently selected from the group consisting of hydrogen; deuterium; halogen; a cyano group; a substituted or unsubstituted C1 to C10 alkyl group; a substituted or unsubstituted C2 to C10 alkenyl group; a substituted or unsubstituted C2 to C10 alkynyl group; a substituted or unsubstituted C1 to C10 alkoxy group; a substituted or unsubstituted C3 to C10 cycloalkyl group; a substituted or unsubstituted C2 to C10 heterocycloalkyl group; a substituted or unsubstituted C6 to C10 aryl group; a substituted or unsubstituted C2 to C10 heteroaryl group; and the group represented by Structural Formula 2, or two or more groups adjacent to each other bond to each other to form a substituted or unsubstituted C6 to C10 aromatic hydrocarbon ring or a substituted or unsubstituted C2 to C10 heteroring, and at least one of B1 to B4 may be the group represented by Structural Formula 2.

In one embodiment of the present application, B1 to B4 of Chemical Formula 1 are the same as or different from each other, and may be each independently hydrogen; deuterium; or the group represented by Structural Formula 2.

In one embodiment of the present application, B1 of Chemical Formula 1 is the group represented by Structural Formula 1, and B2 to B4 are the same as or different from each other and may be each independently hydrogen; or deuterium.

In one embodiment of the present application, B2 of Chemical Formula 1 is the group represented by Structural Formula 1, and B1, B3 and B4 are the same as or different from each other and may be each independently hydrogen; or deuterium.

In one embodiment of the present application, B3 of Chemical Formula 1 is the group represented by Structural Formula 1, and B1, B2 and B4 are the same as or different from each other and may be each independently hydrogen; or deuterium.

In one embodiment of the present application, B4 of Chemical Formula 1 is the group represented by Structural Formula 1, and B1 to B3 are the same as or different from each other and may be each independently hydrogen; or deuterium.

In one embodiment of the present application, in Structural Formula 1 of Chemical Formula 1, R1 and R2 are the same as or different from each other, and each independently selected from the group consisting of hydrogen; deuterium; halogen; a cyano group; a substituted or unsubstituted C1 to C40 alkyl group; a substituted or unsubstituted C2 to C40 alkenyl group; a substituted or unsubstituted C2 to C40 alkynyl group; a substituted or unsubstituted C1 to C40 alkoxy group; a substituted or unsubstituted C3 to C40 cycloalkyl group; a substituted or unsubstituted C2 to C40 heterocycloalkyl group; a substituted or unsubstituted C6 to C40 aryl group; a substituted or unsubstituted C2 to C40 heteroaryl group; —P(═O)R101R102 and —SiR101R102R103, or two or more groups adjacent to each other bond to each other to form a substituted or unsubstituted C6 to C40 aromatic hydrocarbon ring or a substituted or unsubstituted C2 to C40 heteroring, and R101, R102 and R103 are the same as or different from each other and may be each independently a substituted or unsubstituted C1 to C40 alkyl group; a substituted or unsubstituted C6 to C40 aryl group; or a substituted or unsubstituted C2 to C40 heteroaryl group.

In one embodiment of the present application, in Structural Formula 1 of Chemical Formula 1, R1 and R2 are the same as or different from each other, and each independently selected from the group consisting of hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R101R102 and —SiR101R102R103, or two or more groups adjacent to each other bond to each other to form a substituted or unsubstituted C6 to C30 aromatic hydrocarbon ring or a substituted or unsubstituted C2 to C30 heteroring, and R101, R102 and R103 are the same as or different from each other and may be each independently a substituted or unsubstituted C1 to C30 alkyl group; a substituted or unsubstituted C6 to C30 aryl group; or a substituted or unsubstituted C2 to C30 heteroaryl group.

In one embodiment of the present application, in Structural Formula 1 of Chemical Formula 1, R1 and R2 are the same as or different from each other, and each independently selected from the group consisting of hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; and a substituted or unsubstituted C2 to C20 heteroaryl group, or two or more groups adjacent to each other may bond to each other to form a substituted or unsubstituted C6 to C20 aromatic hydrocarbon ring or a substituted or unsubstituted C2 to C20 heteroring.

In one embodiment of the present application, in Structural Formula 1 of Chemical Formula 1, R1 and R2 are the same as or different from each other, and each independently selected from the group consisting of hydrogen; deuterium; halogen; a cyano group; a substituted or unsubstituted C1 to C10 alkyl group; a substituted or unsubstituted C2 to C10 alkenyl group; a substituted or unsubstituted C2 to C10 alkynyl group; a substituted or unsubstituted C1 to C10 alkoxy group; a substituted or unsubstituted C3 to C10 cycloalkyl group; a substituted or unsubstituted C2 to C10 heterocycloalkyl group; a substituted or unsubstituted C6 to C10 aryl group; and a substituted or unsubstituted C2 to C10 heteroaryl group, or two or more groups adjacent to each other may bond to each other to form a substituted or unsubstituted C6 to C10 aromatic hydrocarbon ring or a substituted or unsubstituted C2 to C10 heteroring.

In one embodiment of the present application, in Structural Formula 1 of Chemical Formula 1, R1 and R2 are the same as or different from each other, and may be each independently hydrogen; deuterium; or a substituted or unsubstituted phenyl group.

In one embodiment of the present application, in Structural Formula 2 of Chemical Formula 1, X1 may be O.

In one embodiment of the present application, in Structural Formula 2 of Chemical Formula 1, X1 may be S.

In one embodiment of the present application, in Structural Formula 2 of Chemical Formula 1, L1 to L4 are the same as or different from each other, and may be each independently a single bond; a substituted or unsubstituted C6 to C40 arylene group; or a substituted or unsubstituted C2 to C40 heteroarylene group.

In one embodiment of the present application, in Structural Formula 2 of Chemical Formula 1, L1 to L4 are the same as or different from each other, and may be each independently a single bond; a substituted or unsubstituted C6 to C30 arylene group; or a substituted or unsubstituted C2 to C30 heteroarylene group.

In one embodiment of the present application, in Structural Formula 2 of Chemical Formula 1, L1 to L4 are the same as or different from each other, and may be each independently a single bond; a substituted or unsubstituted C6 to C20 arylene group; or a substituted or unsubstituted C2 to C20 heteroarylene group.

In one embodiment of the present application, in Structural Formula 2 of Chemical Formula 1, L1 to L4 are the same as or different from each other, and may be each independently a single bond; a substituted or unsubstituted C6 to C10 arylene group; or a substituted or unsubstituted C2 to C10 heteroarylene group.

In one embodiment of the present application, in Structural Formula 2 of Chemical Formula 1, L1 to L4 are the same as or different from each other, and may be each independently a single bond; a substituted or unsubstituted phenylene group; a substituted or unsubstituted biphenylene group; or a substituted or unsubstituted naphthylene group.

In one embodiment of the present application, in Structural Formula 2 of Chemical Formula 1, Y1 is N, and Y2 and Y3 may be CH.

In another embodiment, in Structural Formula 2 of Chemical Formula 1, Y1 and Y2 are N, and Y3 may be CH.

In another embodiment, in Structural Formula 2 of Chemical Formula 1, Y1 and Y3 are N, and Y2 may be CH.

In another embodiment, in Structural Formula 2 of Chemical Formula 1, Y1 is CH, and Y2 and Y3 may be N.

In another embodiment, in Structural Formula 2 of Chemical Formula 1, Y1 and Y2 are CH, and Y3 may be N.

In another embodiment, in Structural Formula 2 of Chemical Formula 1, Y1 and Y3 are CH, and Y2 may be N.

In another embodiment, in Structural Formula 2 of Chemical Formula 1, Y1 to Y3 may be N.

In one embodiment of the present application, in Structural Formula 2 of Chemical Formula 1, Ar1 may be a substituted or unsubstituted C6 to C40 aryl group; or a substituted or unsubstituted C2 to C40 heteroaryl group.

In one embodiment of the present application, in Structural Formula 2 of Chemical Formula 1, Ar1 may be a substituted or unsubstituted C6 to C30 aryl group; or a substituted or unsubstituted C2 to C30 heteroaryl group.

In one embodiment of the present application, in Structural Formula 2 of Chemical Formula 1, Ar1 may be a substituted or unsubstituted C6 to C20 aryl group; or a substituted or unsubstituted C2 to C20 heteroaryl group.

In one embodiment of the present application, in Structural Formula 2 of Chemical Formula 1, Ar1 may be a substituted or unsubstituted C6 to C10 aryl group; or a substituted or unsubstituted C2 to C10 heteroaryl group.

In one embodiment of the present application, in Structural Formula 2 of Chemical Formula 1, Ar1 may be a substituted or unsubstituted phenyl group; a substituted or unsubstituted biphenyl group; a substituted or unsubstituted naphthyl group; or a substituted or unsubstituted terphenyl group.

In one embodiment of the present application, in Structural Formula 2 of Chemical Formula 1, R3 and R4 are the same as or different from each other, and each independently selected from the group consisting of hydrogen; deuterium; halogen; a cyano group; a substituted or unsubstituted C1 to C40 alkyl group; a substituted or unsubstituted C2 to C40 alkenyl group; a substituted or unsubstituted C2 to C40 alkynyl group; a substituted or unsubstituted C1 to C40 alkoxy group; a substituted or unsubstituted C3 to C40 cycloalkyl group; a substituted or unsubstituted C2 to C40 heterocycloalkyl group; a substituted or unsubstituted C6 to C40 aryl group; a substituted or unsubstituted C2 to C40 heteroaryl group; —P(═O)R101R102 and —SiR101R102R103, or two or more groups adjacent to each other bond to each other to form a substituted or unsubstituted C6 to C40 aromatic hydrocarbon ring or a substituted or unsubstituted C2 to C40 heteroring, and R101, R102 and R103 are the same as or different from each other and may be each independently a substituted or unsubstituted C1 to C40 alkyl group; a substituted or unsubstituted C6 to C40 aryl group; or a substituted or unsubstituted C2 to C40 heteroaryl group.

In one embodiment of the present application, in Structural Formula 2 of Chemical Formula 1, R3 and R4 are the same as or different from each other, and each independently selected from the group consisting of hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R101R102 and —SiR101R102R103, or two or more groups adjacent to each other bond to each other to form a substituted or unsubstituted C6 to C30 aromatic hydrocarbon ring or a substituted or unsubstituted C2 to C30 heteroring, and R101, R102 and R103 are the same as or different from each other and may be each independently a substituted or unsubstituted C1 to C30 alkyl group; a substituted or unsubstituted C6 to C30 aryl group; or a substituted or unsubstituted C2 to C30 heteroaryl group.

In one embodiment of the present application, in Structural Formula 2 of Chemical Formula 1, R3 and R4 are the same as or different from each other, and each independently selected from the group consisting of hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; and a substituted or unsubstituted C2 to C20 heteroaryl group, or two or more groups adjacent to each other may bond to each other to form a substituted or unsubstituted C6 to C20 aromatic hydrocarbon ring or a substituted or unsubstituted C2 to C20 heteroring.

In one embodiment of the present application, in Structural Formula 2 of Chemical Formula 1, R3 and R4 are the same as or different from each other, and each independently selected from the group consisting of hydrogen; deuterium; halogen; a cyano group; a substituted or unsubstituted C1 to C10 alkyl group; a substituted or unsubstituted C2 to C10 alkenyl group; a substituted or unsubstituted C2 to C10 alkynyl group; a substituted or unsubstituted C1 to C10 alkoxy group; a substituted or unsubstituted C3 to C10 cycloalkyl group; a substituted or unsubstituted C2 to C10 heterocycloalkyl group; a substituted or unsubstituted C6 to C10 aryl group; and a substituted or unsubstituted C2 to C10 heteroaryl group, or two or more groups adjacent to each other may bond to each other to form a substituted or unsubstituted C6 to C10 aromatic hydrocarbon ring or a substituted or unsubstituted C2 to C10 heteroring.

In one embodiment of the present application, in Structural Formula 2 of Chemical Formula 1, R3 and R4 are the same as or different from each other, and may be each independently hydrogen; deuterium; a substituted or unsubstituted phenyl group; a substituted or unsubstituted biphenyl group; a substituted or unsubstituted naphthyl group; or a substituted or unsubstituted terphenyl group.

In one embodiment of the present application, Chemical Formula 1 may be represented by any one of the following Chemical Formula 1-1 or 1-2.

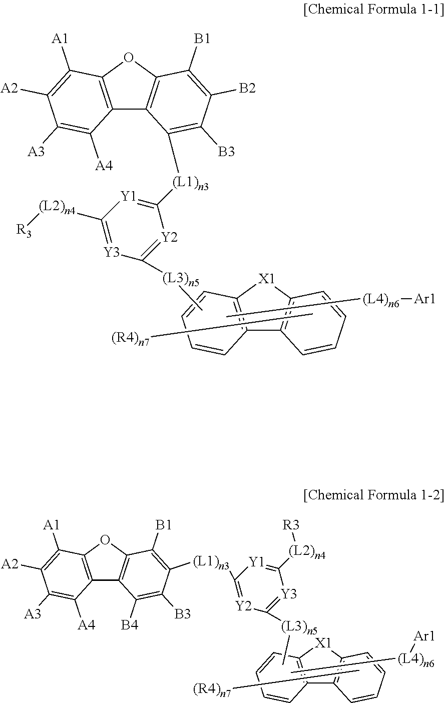

In Chemical Formulae 1-1 and 1-2,
A1 to A4, B1 to B4, X1, L1 to L4, Y1 to Y3, R3, R4, n3 to n7 and Ar1 have the same definitions as in Chemical Formula 1.

In one embodiment of the present application, Structural Formula 2 may be represented by any one of the following Structural Formulae 2-1 to 2-8.

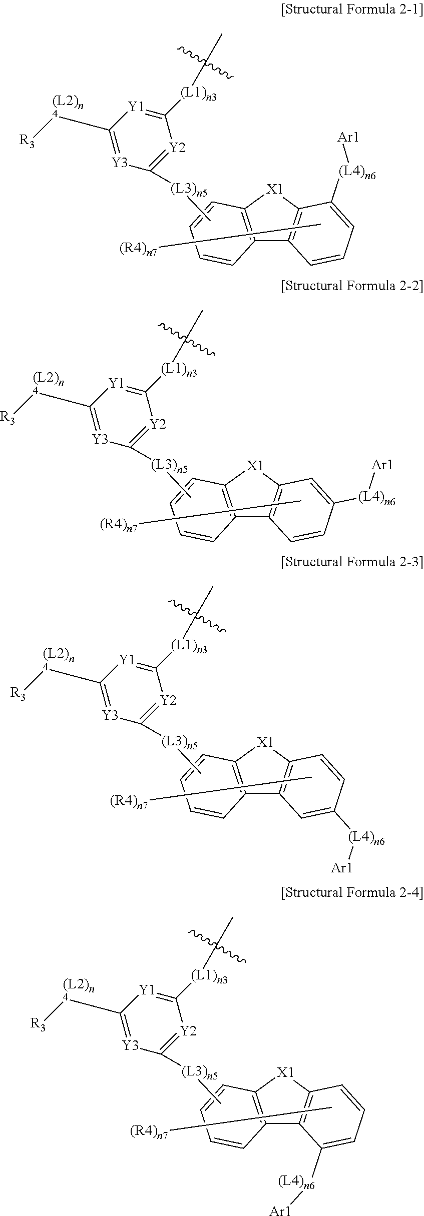

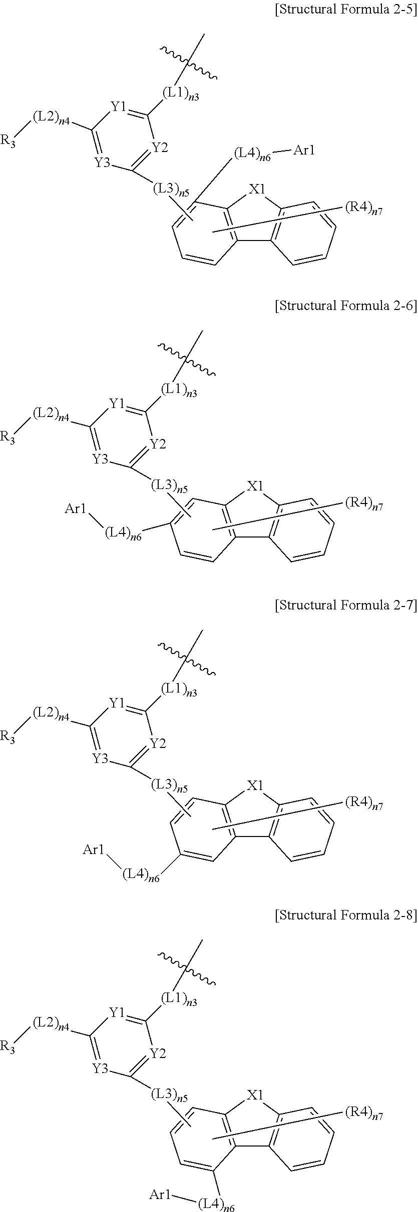

In Structural Formulae 2-1 to 2-8,
L1 to L4, X1, Y1 to Y3, R3, R4, n3 to n7 and Ar1 have the same definitions as in Chemical Formula 1.

In one embodiment of the present application, the heterocyclic compound represented by Chemical Formula 1 may not include deuterium as a substituent, or may have a deuterium content of, for example, greater than 0%, 1% or greater, 10% or greater, 20% or greater, 30% or greater, 40% or greater or 50% or greater, and 100% or less, 90% or less, 80% or less, 70% or less or 60% or less with respect to the total number of hydrogen atoms and deuterium atoms.

In one embodiment of the present application, the heterocyclic compound represented by Chemical Formula 1 may not include deuterium, or may have a deuterium content of 1% to 100% with respect to the total number of hydrogen atoms and deuterium atoms.

In one embodiment of the present application, the heterocyclic compound represented by Chemical Formula 1 may not include deuterium, or may have a deuterium content of 10% to 100% with respect to the total number of hydrogen atoms and deuterium atoms.

In embodiment of the present application, the heterocyclic compound represented by Chemical Formula 1 may not include deuterium, or may have a deuterium content of 20% to 90% with respect to the total number of hydrogen atoms and deuterium atoms.

In one embodiment of the present application, the heterocyclic compound represented by Chemical Formula 1 may not include deuterium, or may have a deuterium content of 30% to 80% with respect to the total number of hydrogen atoms and deuterium atoms.

In embodiment of the present application, the heterocyclic compound represented by Chemical Formula 1 may not include deuterium, or may have a deuterium content of 40% to 70% with respect to the total number of hydrogen atoms and deuterium atoms.

In one embodiment of the present application, the heterocyclic compound represented by Chemical Formula 1 may have a deuterium content of 0% or greater, 1% or greater, 5% or greater, 10% or greater, 15% or greater, 20% or greater, 25% or greater, 30% or greater, 35% or greater, 40% or greater, 45% or greater or 50% or greater, and 100% or less, 95% or less, 90% or less, 85% or less, 80% or less, 75% or less, 70% or less, 65% or less or 60% or less based on the total number of hydrogen atoms and deuterium atoms.

One embodiment of the present application provides a heterocyclic compound, wherein Chemical Formula 1 is represented by any one of the following compounds. In addition, in one embodiment of the present application, the following compounds are just one example, and the present application is not limited thereto and may include other compounds included in Chemical Formula 1 including additional substituents.

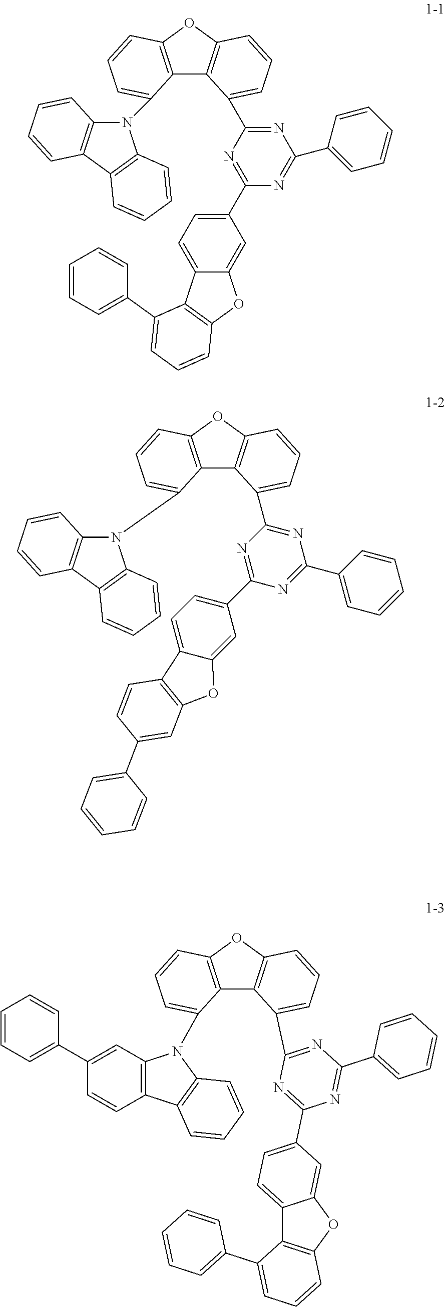 [SMILES: c1ccc(-c2nc(-c3ccc4c(c3)oc3cccc(-c5ccccc5)c34)nc(-c3cccc4oc5cccc(-n6c7ccccc7c7ccccc76)c5c34)n2)cc1 ; c1ccc(-c2ccc3c(c2)oc2cc(-c4nc(-c5ccccc5)nc(-c5cccc6oc7cccc(-n8c9ccccc9c9ccccc98)c7c56)n4)ccc23)cc1 ; c1ccc(-c2ccc3c4ccccc4n(-c4cccc5oc6cccc(-c7nc(-c8ccccc8)nc(-c8ccc9c(c8)oc8cccc(-c%10ccccc%10)c89)n7)c6c45)c3c2)cc1]

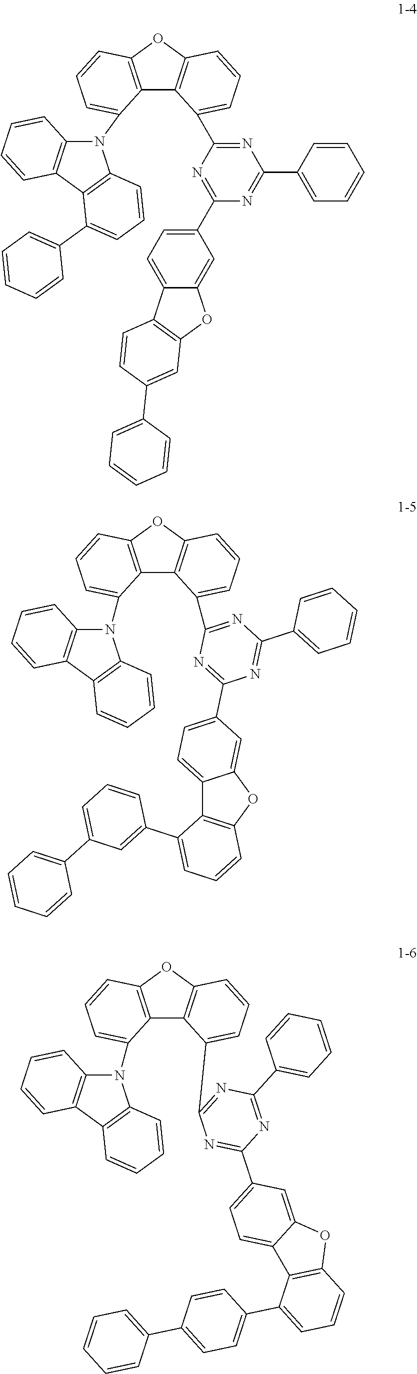

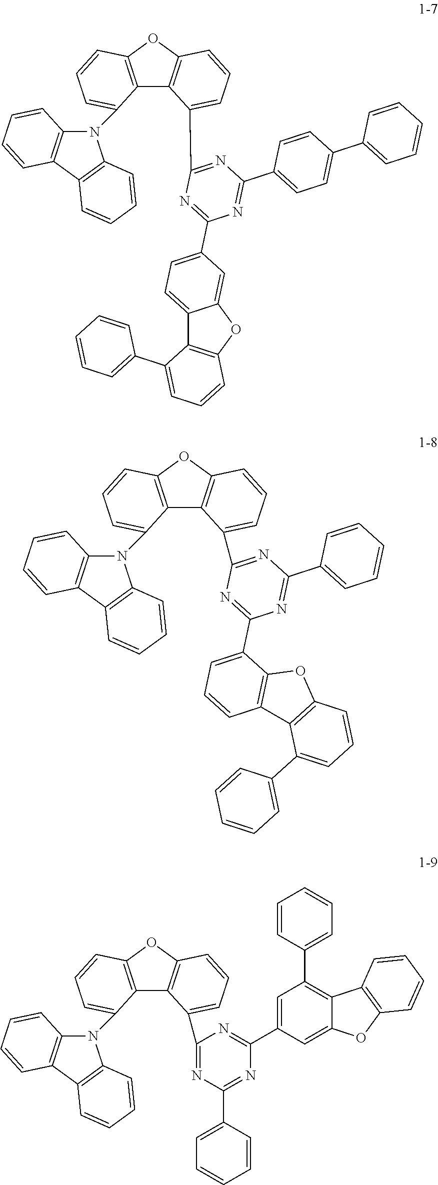 [SMILES: c1ccc(-c2ccc(-c3nc(-c4ccc5c(c4)oc4cccc(-c6ccccc6)c45)nc(-c4cccc5oc6cccc(-n7c8ccccc8c8ccccc87)c6c45)n3)cc2)cc1 ; c1ccc(-c2nc(-c3cccc4c3oc3cccc(-c5ccccc5)c34)nc(-c3cccc4oc5cccc(-n6c7ccccc7c7ccccc76)c5c34)n2)cc1 ; c1ccc(-c2nc(-c3cc(-c4ccccc4)c4c(c3)oc3ccccc34)nc(-c3cccc4oc5cccc(-n6c7ccccc7c7ccccc76)c5c34)n2)cc1]

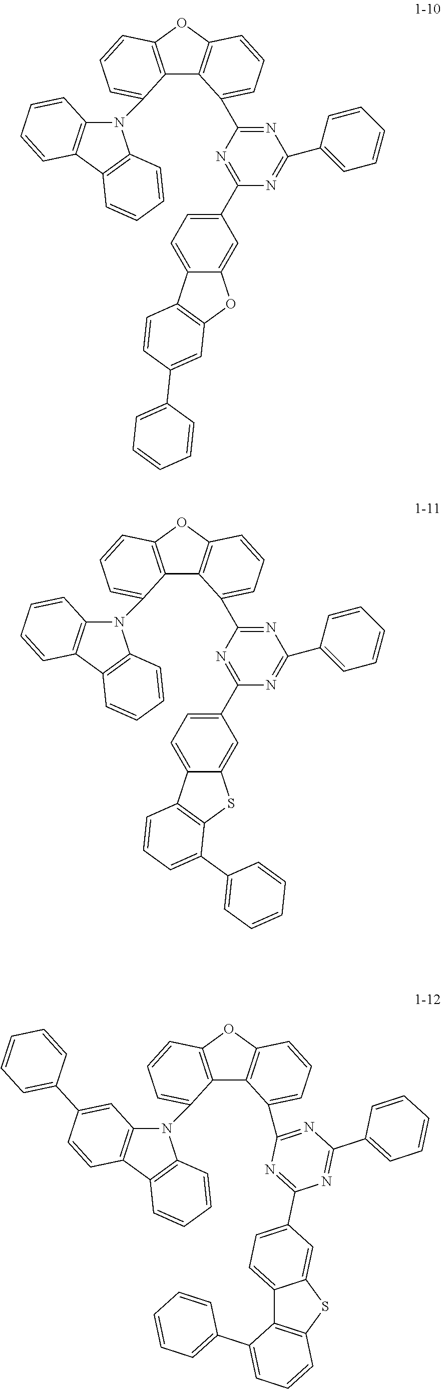 [SMILES: c1ccc(-c2ccc3c(c2)oc2cc(-c4nc(-c5ccccc5)nc(-c5cccc6oc7cccc(-n8c9ccccc9c9ccccc98)c7c56)n4)ccc23)cc1 ; c1ccc(-c2nc(-c3ccc4c(c3)sc3c(-c5ccccc5)cccc34)nc(-c3cccc4oc5cccc(-n6c7ccccc7c7ccccc76)c5c34)n2)cc1 ; c1ccc(-c2ccc3c4ccccc4n(-c4cccc5oc6cccc(-c7nc(-c8ccccc8)nc(-c8ccc9c(c8)sc8cccc(-c%10ccccc%10)c89)n7)c6c45)c3c2)cc1]

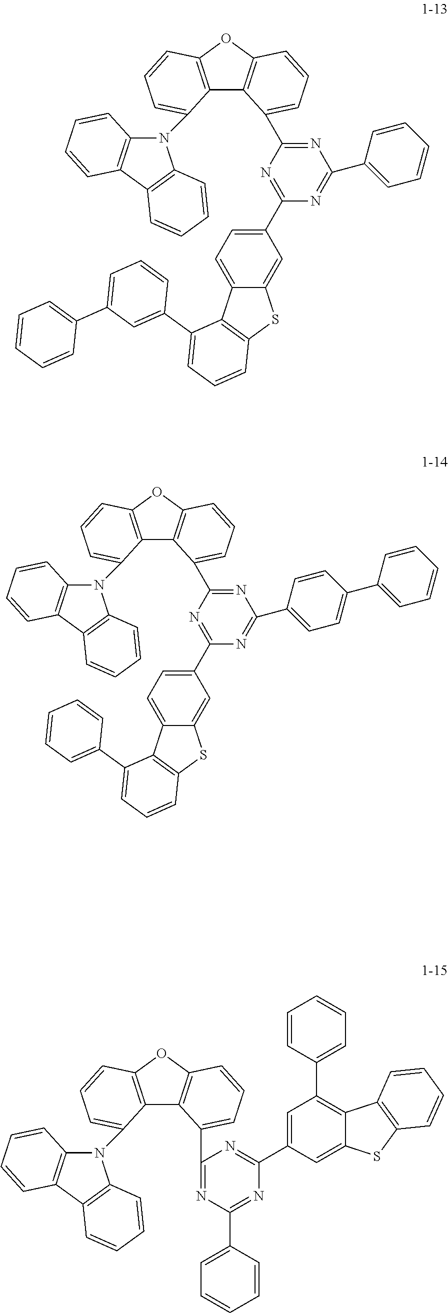 [SMILES: c1ccc(-c2cccc(-c3cccc4sc5cc(-c6nc(-c7ccccc7)nc(-c7cccc8oc9cccc(-n%10c%11ccccc%11c%11ccccc%11%10)c9c78)n6)ccc5c34)c2)cc1 ; c1ccc(-c2ccc(-c3nc(-c4ccc5c(c4)sc4cccc(-c6ccccc6)c45)nc(-c4cccc5oc6cccc(-n7c8ccccc8c8ccccc87)c6c45)n3)cc2)cc1 ; c1ccc(-c2nc(-c3cc(-c4ccccc4)c4c(c3)sc3ccccc34)nc(-c3cccc4oc5cccc(-n6c7ccccc7c7ccccc76)c5c34)n2)cc1]

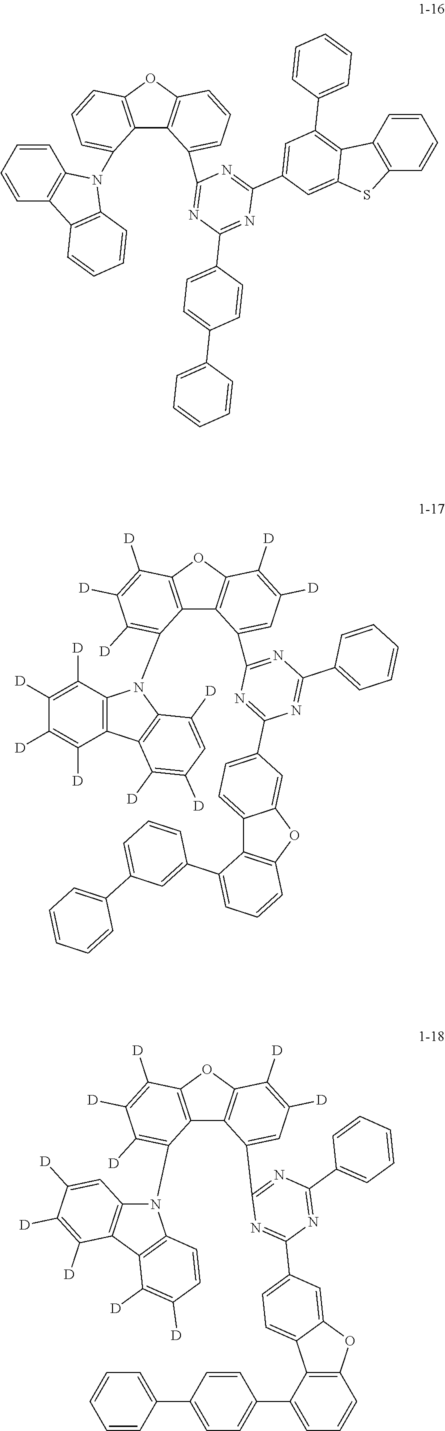 [SMILES: c1ccc(-c2ccc(-c3nc(-c4cc(-c5ccccc5)c5c(c4)sc4ccccc45)nc(-c4cccc5oc6cccc(-n7c8ccccc8c8ccccc87)c6c45)n3)cc2)cc1 ; [2H]c1cc(-c2nc(-c3ccccc3)nc(-c3ccc4c(c3)oc3cccc(-c5cccc(-c6ccccc6)c5)c34)n2)c2c(oc3c([2H])c([2H])c([2H])c(-n4c5c([2H])cc([2H])c([2H])c5c5c([2H])c([2H])c([2H])c([2H])c54)c32)c1[2H] ; [2H]c1cc2c(c([2H])c1[2H])c1c([2H])c([2H])ccc1n2-c1c([2H])c([2H])c([2H])c2oc3c([2H])c([2H])cc(-c4nc(-c5ccccc5)nc(-c5ccc6c(c5)oc5cccc(-c7ccc(-c8ccccc8)cc7)c56)n4)c3c12]

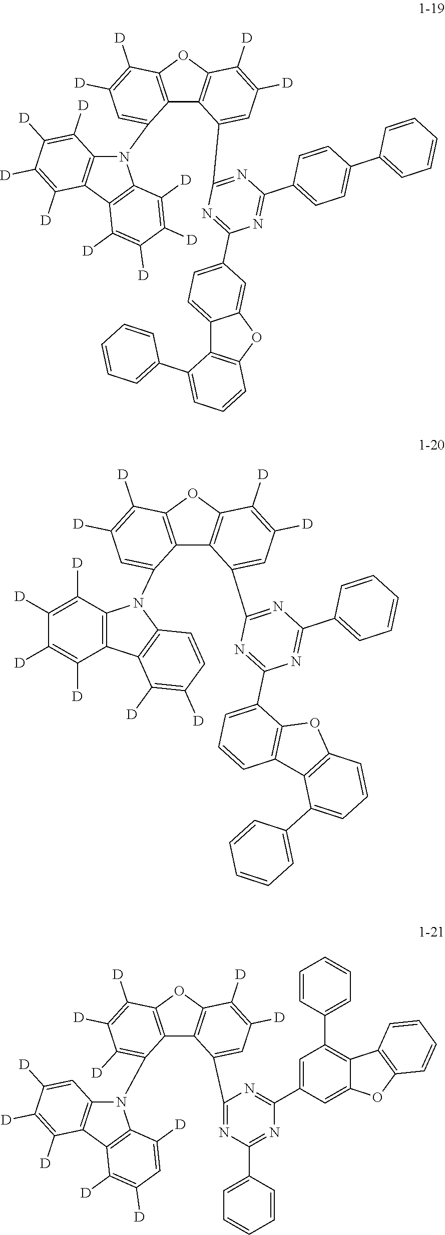 [SMILES: [2H]c1cc(-c2nc(-c3ccc(-c4ccccc4)cc3)nc(-c3ccc4c(c3)oc3cccc(-c5ccccc5)c34)n2)c2c(oc3c([2H])c([2H])cc(-n4c5c([2H])c([2H])c([2H])c([2H])c5c5c([2H])c([2H])c([2H])c([2H])c54)c32)c1[2H] ; [2H]c1cc(-c2nc(-c3ccccc3)nc(-c3cccc4c3oc3cccc(-c5ccccc5)c34)n2)c2c(oc3c([2H])c([2H])cc(-n4c5ccc([2H])c([2H])c5c5c([2H])c([2H])c([2H])c([2H])c54)c32)c1[2H] ; [2H]c1cc2c(c([2H])c1[2H])c1c([2H])c([2H])cc([2H])c1n2-c1c([2H])c([2H])c([2H])c2oc3c([2H])c([2H])cc(-c4nc(-c5ccccc5)nc(-c5cc(-c6ccccc6)c6c(c5)oc5ccccc56)n4)c3c12]

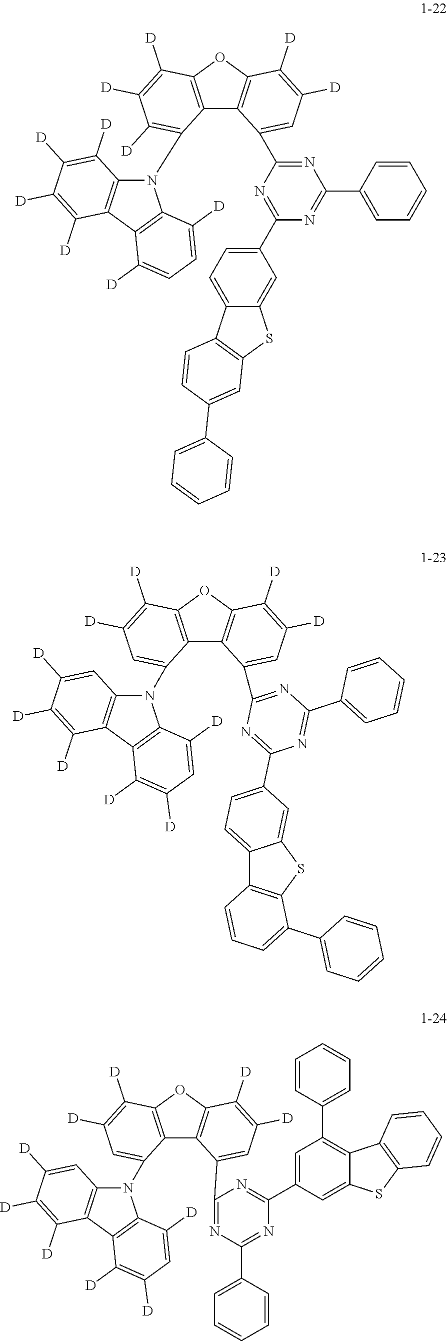 [SMILES: [2H]c1cc(-c2nc(-c3ccccc3)nc(-c3ccc4c(c3)sc3cc(-c5ccccc5)ccc34)n2)c2c(oc3c([2H])c([2H])c([2H])c(-n4c5c([2H])ccc([2H])c5c5c([2H])c([2H])c([2H])c([2H])c54)c32)c1[2H] ; [2H]c1cc2c(c([2H])c1[2H])c1c([2H])c([2H])cc([2H])c1n2-c1cc([2H])c([2H])c2oc3c([2H])c([2H])cc(-c4nc(-c5ccccc5)nc(-c5ccc6c(c5)sc5c(-c7ccccc7)cccc56)n4)c3c12 ; [2H]c1cc2c(c([2H])c1[2H])c1c([2H])c([2H])cc([2H])c1n2-c1cc([2H])c([2H])c2oc3c([2H])c([2H])cc(-c4nc(-c5ccccc5)nc(-c5cc(-c6ccccc6)c6c(c5)sc5ccccc56)n4)c3c12]

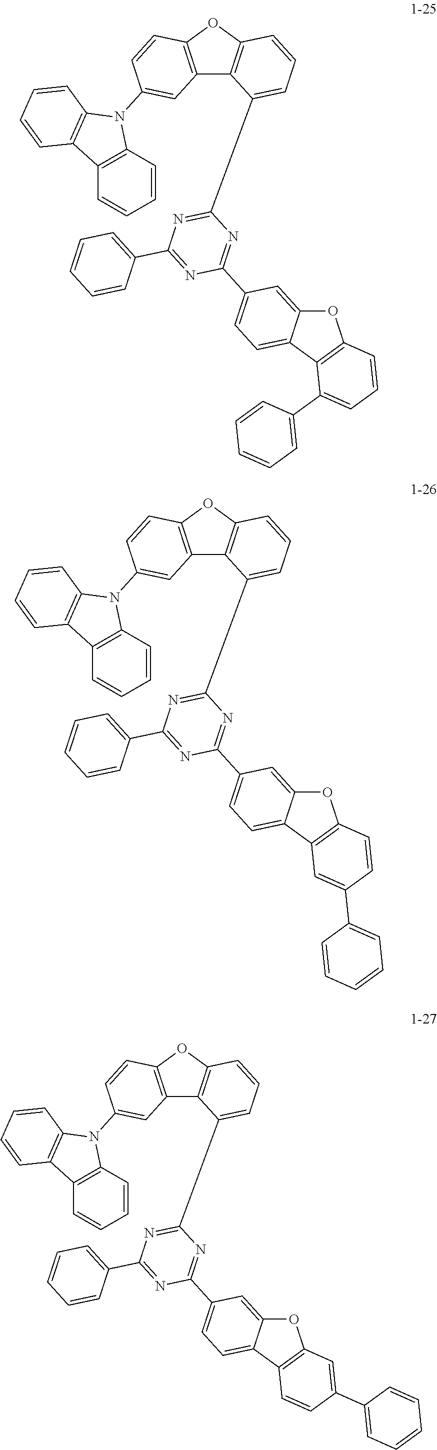 [SMILES: c1ccc(-c2nc(-c3ccc4c(c3)oc3cccc(-c5ccccc5)c34)nc(-c3cccc4oc5ccc(-n6c7ccccc7c7ccccc76)cc5c34)n2)cc1 ; c1ccc(-c2ccc3oc4cc(-c5nc(-c6ccccc6)nc(-c6cccc7oc8ccc(-n9c%10ccccc%10c%10ccccc%109)cc8c67)n5)ccc4c3c2)cc1 ; c1ccc(-c2ccc3c(c2)oc2cc(-c4nc(-c5ccccc5)nc(-c5cccc6oc7ccc(-n8c9ccccc9c9ccccc98)cc7c56)n4)ccc23)cc1]

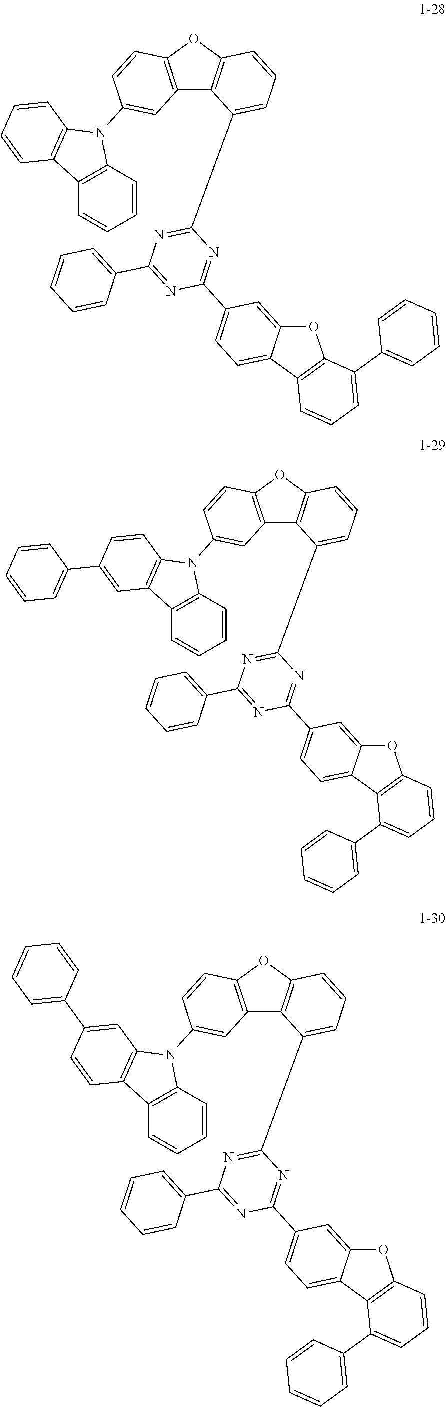 [SMILES: c1ccc(-c2nc(-c3ccc4c(c3)oc3c(-c5ccccc5)cccc34)nc(-c3cccc4oc5ccc(-n6c7ccccc7c7ccccc76)cc5c34)n2)cc1 ; c1ccc(-c2ccc3c(c2)c2ccccc2n3-c2ccc3oc4cccc(-c5nc(-c6ccccc6)nc(-c6ccc7c(c6)oc6cccc(-c8ccccc8)c67)n5)c4c3c2)cc1 ; c1ccc(-c2ccc3c4ccccc4n(-c4ccc5oc6cccc(-c7nc(-c8ccccc8)nc(-c8ccc9c(c8)oc8cccc(-c%10ccccc%10)c89)n7)c6c5c4)c3c2)cc1]

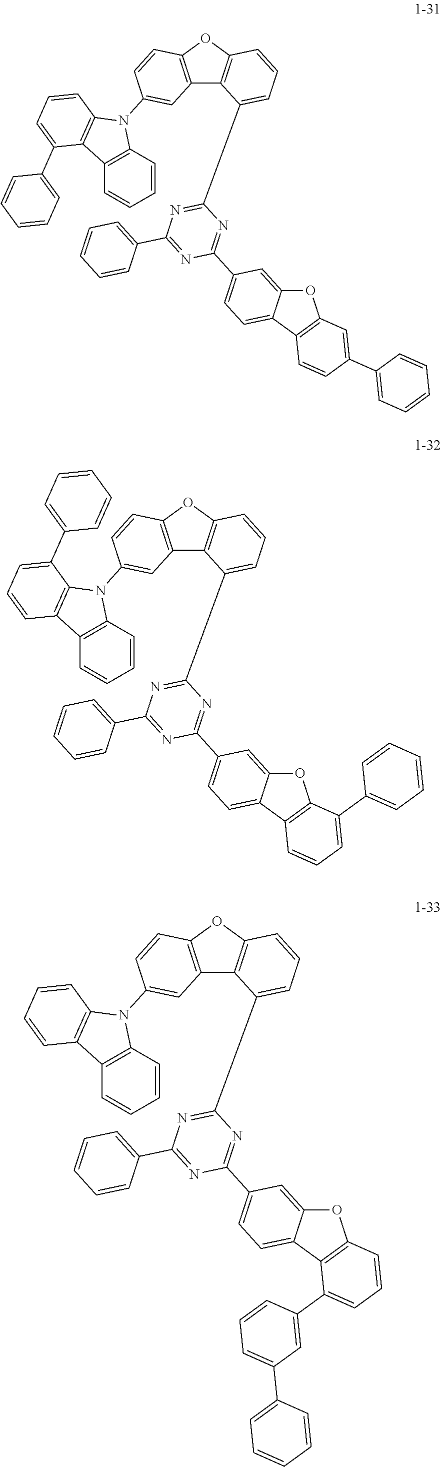 [SMILES: c1ccc(-c2ccc3c(c2)oc2cc(-c4nc(-c5ccccc5)nc(-c5cccc6oc7ccc(-n8c9ccccc9c9c(-c%10ccccc%10)cccc98)cc7c56)n4)ccc23)cc1 ; c1ccc(-c2nc(-c3ccc4c(c3)oc3c(-c5ccccc5)cccc34)nc(-c3cccc4oc5ccc(-n6c7ccccc7c7cccc(-c8ccccc8)c76)cc5c34)n2)cc1 ; c1ccc(-c2cccc(-c3cccc4oc5cc(-c6nc(-c7ccccc7)nc(-c7cccc8oc9ccc(-n%10c%11ccccc%11c%11ccccc%11%10)cc9c78)n6)ccc5c34)c2)cc1]

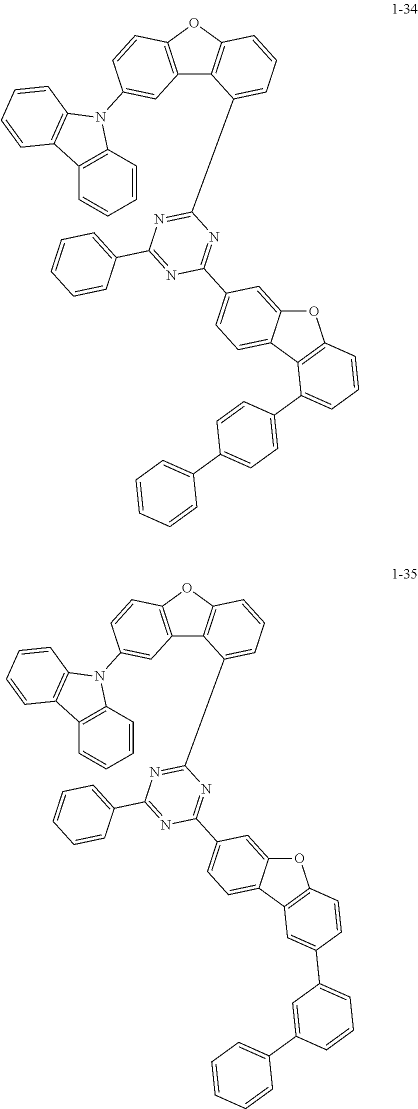 [SMILES: c1ccc(-c2ccc(-c3cccc4oc5cc(-c6nc(-c7ccccc7)nc(-c7cccc8oc9ccc(-n%10c%11ccccc%11c%11ccccc%11%10)cc9c78)n6)ccc5c34)cc2)cc1 ; c1ccc(-c2cccc(-c3ccc4oc5cc(-c6nc(-c7ccccc7)nc(-c7cccc8oc9ccc(-n%10c%11ccccc%11c%11ccccc%11%10)cc9c78)n6)ccc5c4c3)c2)cc1]

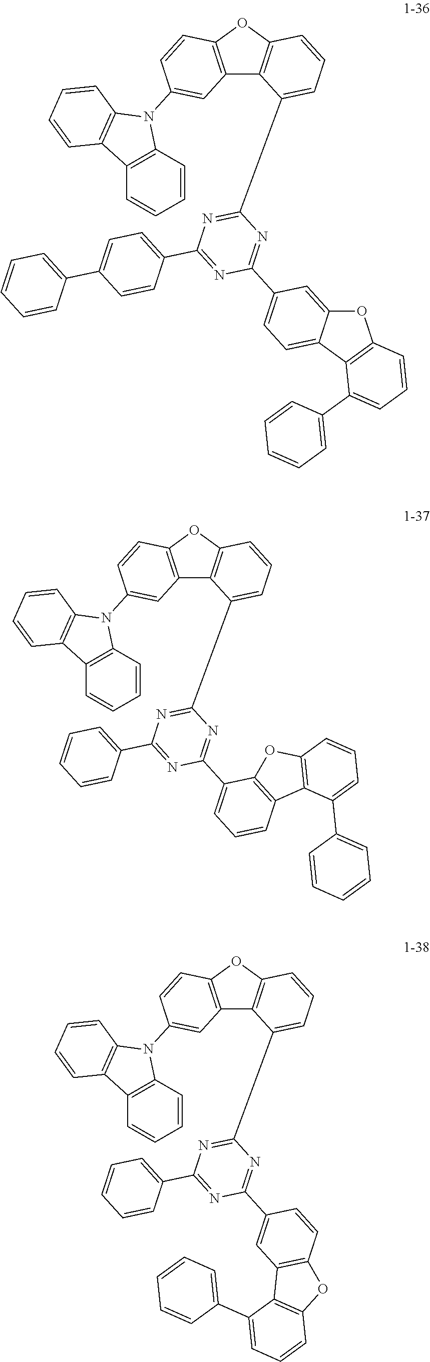 [SMILES: c1ccc(-c2ccc(-c3nc(-c4ccc5c(c4)oc4cccc(-c6ccccc6)c45)nc(-c4cccc5oc6ccc(-n7c8ccccc8c8ccccc87)cc6c45)n3)cc2)cc1 ; c1ccc(-c2nc(-c3cccc4c3oc3cccc(-c5ccccc5)c34)nc(-c3cccc4oc5ccc(-n6c7ccccc7c7ccccc76)cc5c34)n2)cc1 ; c1ccc(-c2nc(-c3ccc4oc5cccc(-c6ccccc6)c5c4c3)nc(-c3cccc4oc5ccc(-n6c7ccccc7c7ccccc76)cc5c34)n2)cc1]

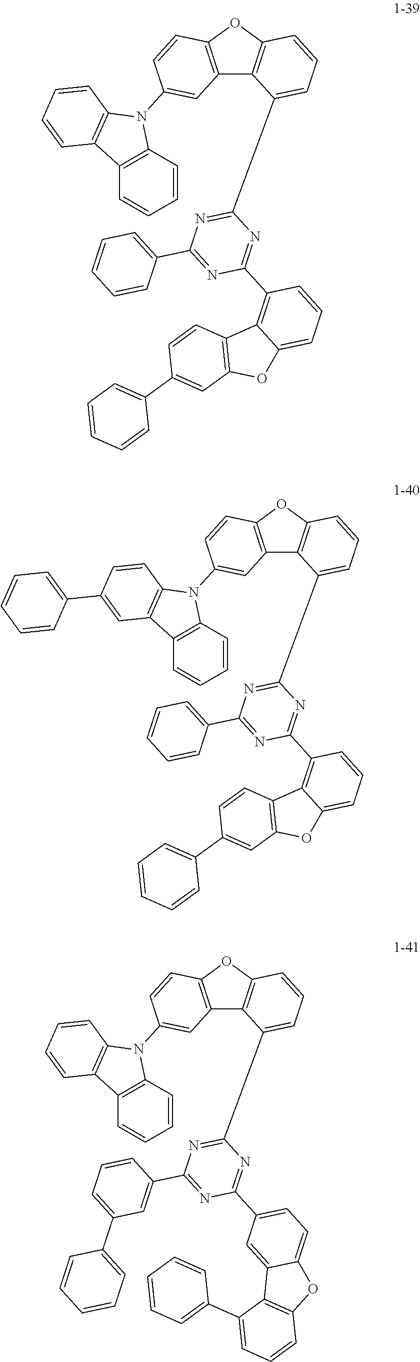 [SMILES: c1ccc(-c2ccc3c(c2)oc2cccc(-c4nc(-c5ccccc5)nc(-c5cccc6oc7ccc(-n8c9ccccc9c9ccccc98)cc7c56)n4)c23)cc1 ; c1ccc(-c2ccc3c(c2)oc2cccc(-c4nc(-c5ccccc5)nc(-c5cccc6oc7ccc(-n8c9ccccc9c9cc(-c%10ccccc%10)ccc98)cc7c56)n4)c23)cc1 ; c1ccc(-c2cccc(-c3nc(-c4ccc5oc6cccc(-c7ccccc7)c6c5c4)nc(-c4cccc5oc6ccc(-n7c8ccccc8c8ccccc87)cc6c45)n3)c2)cc1]

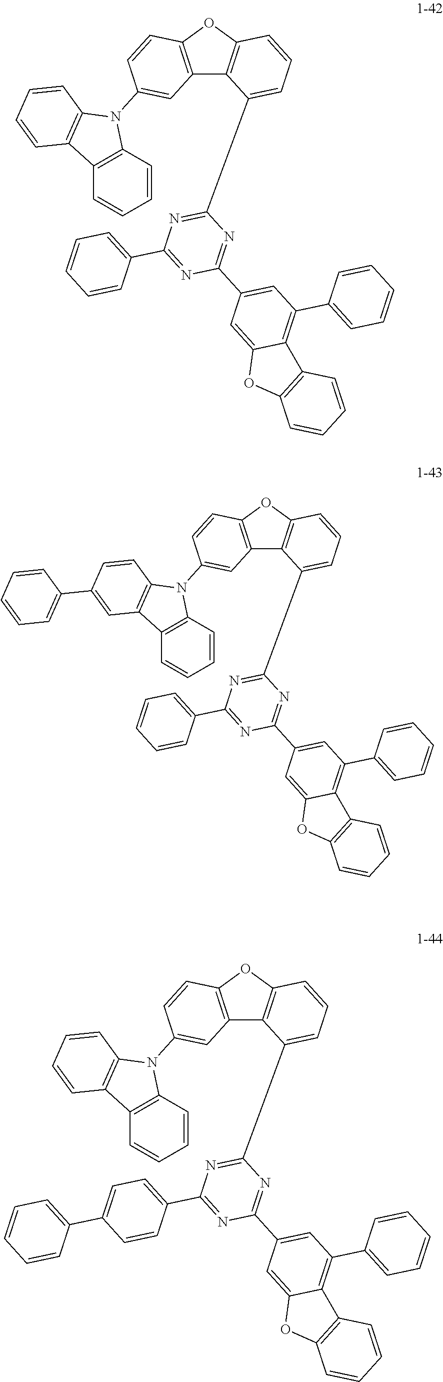 [SMILES: c1ccc(-c2nc(-c3cc(-c4ccccc4)c4c(c3)oc3ccccc34)nc(-c3cccc4oc5ccc(-n6c7ccccc7c7ccccc76)cc5c34)n2)cc1 ; c1ccc(-c2ccc3c(c2)c2ccccc2n3-c2ccc3oc4cccc(-c5nc(-c6ccccc6)nc(-c6cc(-c7ccccc7)c7c(c6)oc6ccccc67)n5)c4c3c2)cc1 ; c1ccc(-c2ccc(-c3nc(-c4cc(-c5ccccc5)c5c(c4)oc4ccccc45)nc(-c4cccc5oc6ccc(-n7c8ccccc8c8ccccc87)cc6c45)n3)cc2)cc1]

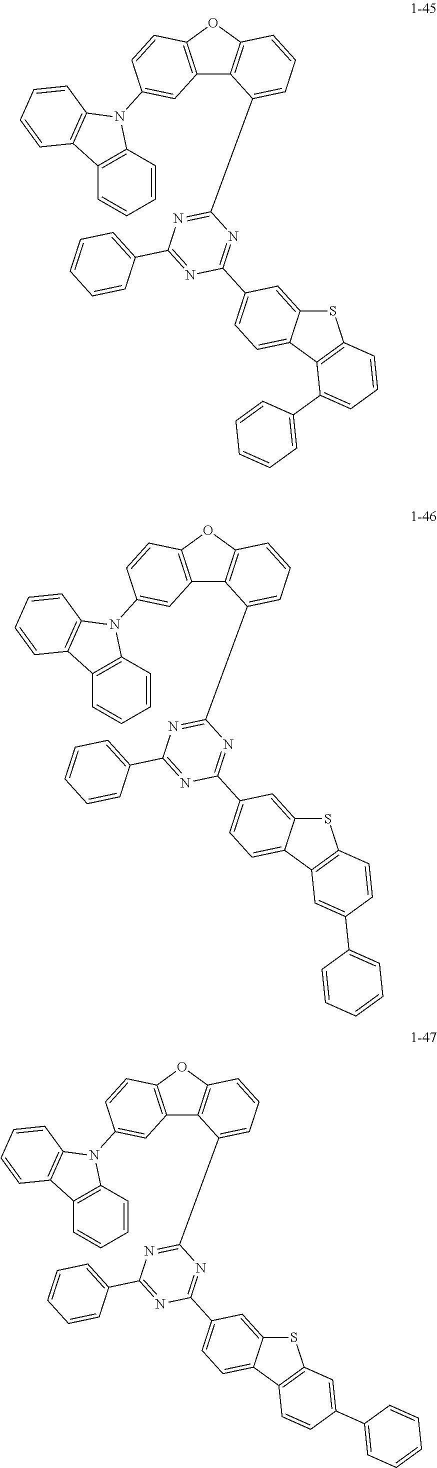 [SMILES: c1ccc(-c2nc(-c3ccc4c(c3)sc3cccc(-c5ccccc5)c34)nc(-c3cccc4oc5ccc(-n6c7ccccc7c7ccccc76)cc5c34)n2)cc1 ; c1ccc(-c2ccc3sc4cc(-c5nc(-c6ccccc6)nc(-c6cccc7oc8ccc(-n9c%10ccccc%10c%10ccccc%109)cc8c67)n5)ccc4c3c2)cc1 ; c1ccc(-c2ccc3c(c2)sc2cc(-c4nc(-c5ccccc5)nc(-c5cccc6oc7ccc(-n8c9ccccc9c9ccccc98)cc7c56)n4)ccc23)cc1]

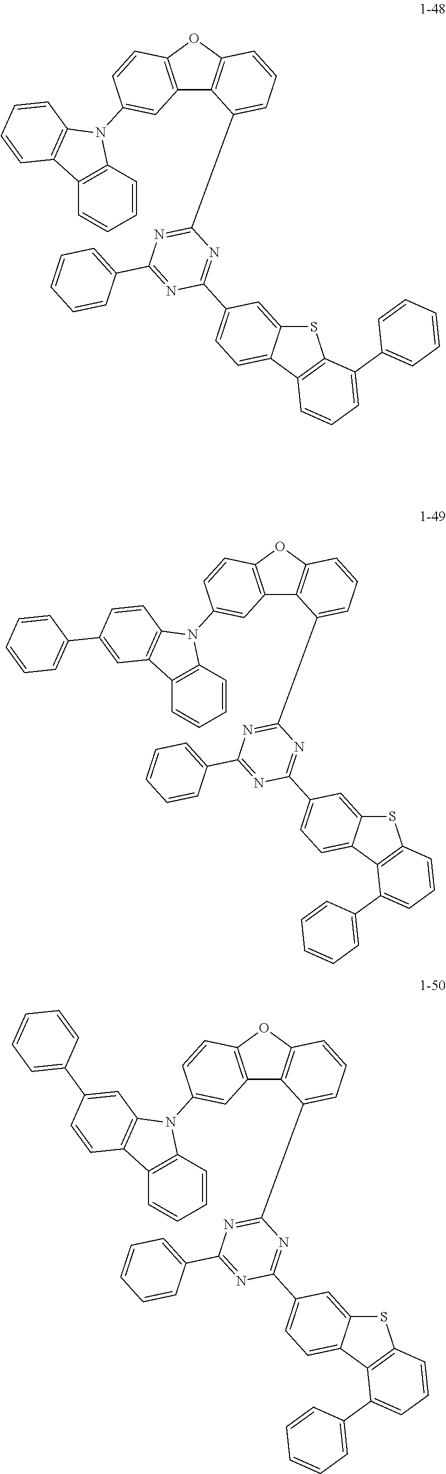 [SMILES: c1ccc(-c2nc(-c3ccc4c(c3)sc3c(-c5ccccc5)cccc34)nc(-c3cccc4oc5ccc(-n6c7ccccc7c7ccccc76)cc5c34)n2)cc1 ; c1ccc(-c2ccc3c(c2)c2ccccc2n3-c2ccc3oc4cccc(-c5nc(-c6ccccc6)nc(-c6ccc7c(c6)sc6cccc(-c8ccccc8)c67)n5)c4c3c2)cc1 ; c1ccc(-c2ccc3c4ccccc4n(-c4ccc5oc6cccc(-c7nc(-c8ccccc8)nc(-c8ccc9c(c8)sc8cccc(-c%10ccccc%10)c89)n7)c6c5c4)c3c2)cc1]

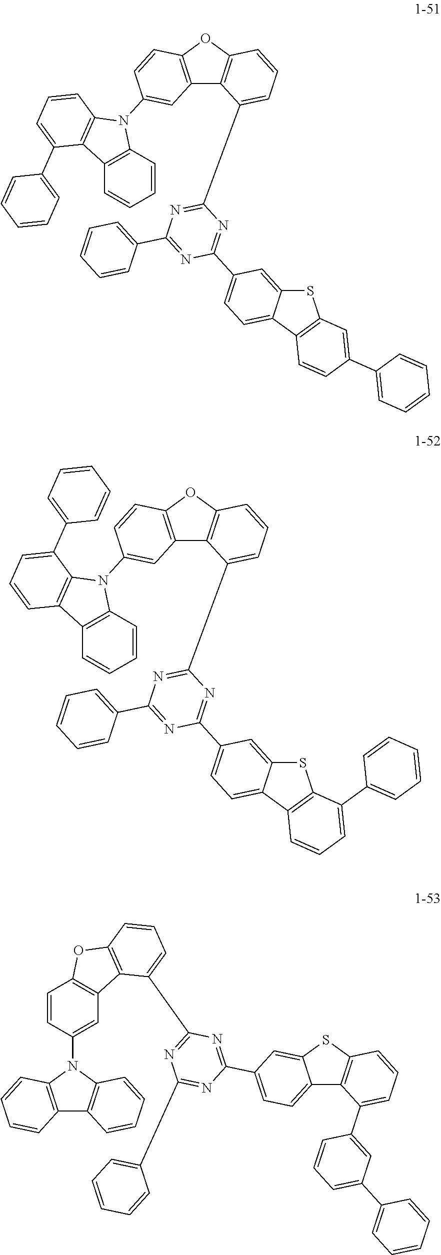 [SMILES: c1ccc(-c2ccc3c(c2)sc2cc(-c4nc(-c5ccccc5)nc(-c5cccc6oc7ccc(-n8c9ccccc9c9c(-c%10ccccc%10)cccc98)cc7c56)n4)ccc23)cc1 ; c1ccc(-c2nc(-c3ccc4c(c3)sc3c(-c5ccccc5)cccc34)nc(-c3cccc4oc5ccc(-n6c7ccccc7c7cccc(-c8ccccc8)c76)cc5c34)n2)cc1 ; c1ccc(-c2cccc(-c3cccc4sc5cc(-c6nc(-c7ccccc7)nc(-c7cccc8oc9ccc(-n%10c%11ccccc%11c%11ccccc%11%10)cc9c78)n6)ccc5c34)c2)cc1]

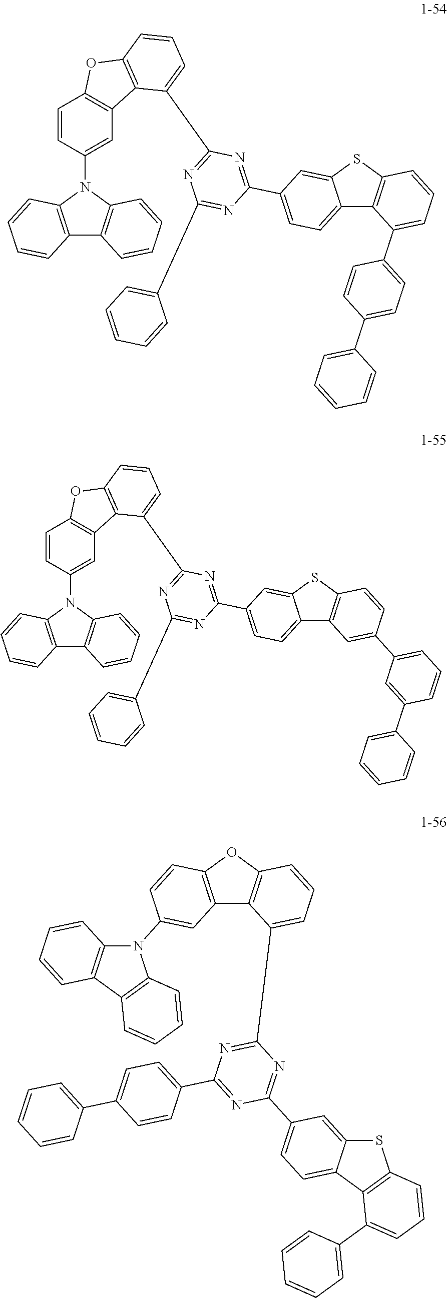 [SMILES: c1ccc(-c2ccc(-c3cccc4sc5cc(-c6nc(-c7ccccc7)nc(-c7cccc8oc9ccc(-n%10c%11ccccc%11c%11ccccc%11%10)cc9c78)n6)ccc5c34)cc2)cc1 ; c1ccc(-c2cccc(-c3ccc4sc5cc(-c6nc(-c7ccccc7)nc(-c7cccc8oc9ccc(-n%10c%11ccccc%11c%11ccccc%11%10)cc9c78)n6)ccc5c4c3)c2)cc1 ; c1ccc(-c2ccc(-c3nc(-c4ccc5c(c4)sc4cccc(-c6ccccc6)c45)nc(-c4cccc5oc6ccc(-n7c8ccccc8c8ccccc87)cc6c45)n3)cc2)cc1]

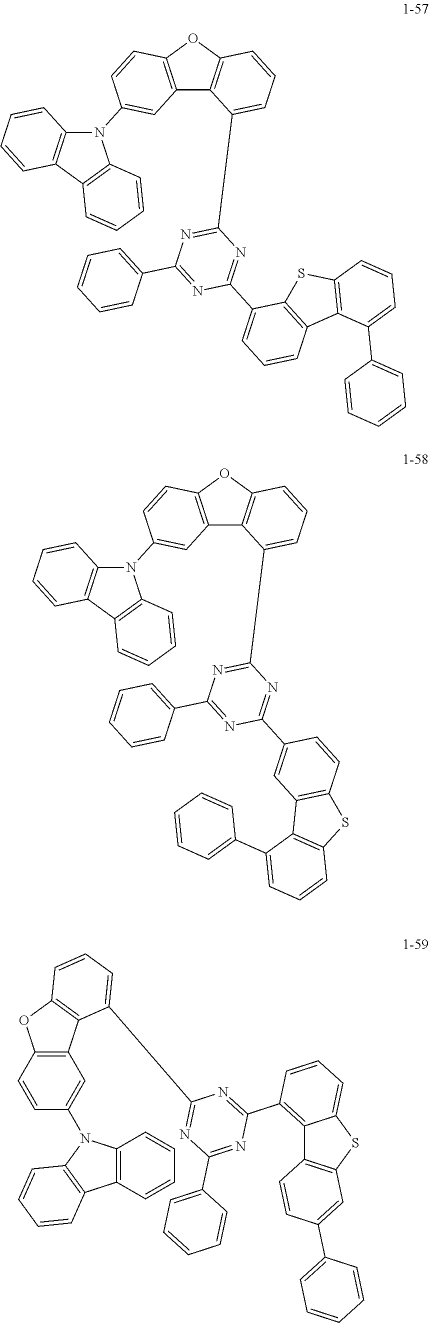 [SMILES: c1ccc(-c2nc(-c3cccc4c3sc3cccc(-c5ccccc5)c34)nc(-c3cccc4oc5ccc(-n6c7ccccc7c7ccccc76)cc5c34)n2)cc1 ; c1ccc(-c2nc(-c3ccc4sc5cccc(-c6ccccc6)c5c4c3)nc(-c3cccc4oc5ccc(-n6c7ccccc7c7ccccc76)cc5c34)n2)cc1 ; c1ccc(-c2ccc3c(c2)sc2cccc(-c4nc(-c5ccccc5)nc(-c5cccc6oc7ccc(-n8c9ccccc9c9ccccc98)cc7c56)n4)c23)cc1]

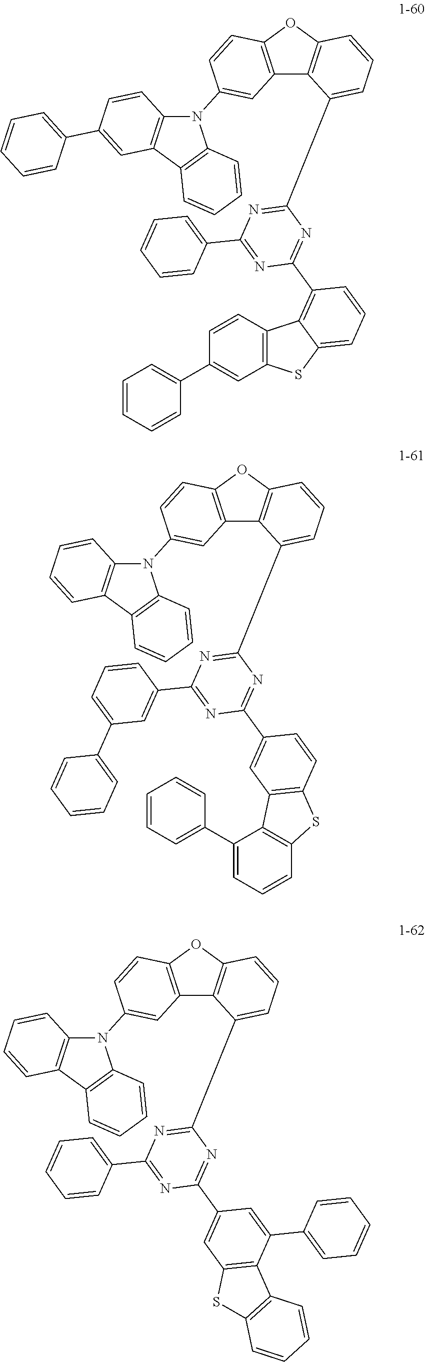 [SMILES: c1ccc(-c2ccc3c(c2)sc2cccc(-c4nc(-c5ccccc5)nc(-c5cccc6oc7ccc(-n8c9ccccc9c9cc(-c%10ccccc%10)ccc98)cc7c56)n4)c23)cc1 ; c1ccc(-c2cccc(-c3nc(-c4ccc5sc6cccc(-c7ccccc7)c6c5c4)nc(-c4cccc5oc6ccc(-n7c8ccccc8c8ccccc87)cc6c45)n3)c2)cc1 ; c1ccc(-c2nc(-c3cc(-c4ccccc4)c4c(c3)sc3ccccc34)nc(-c3cccc4oc5ccc(-n6c7ccccc7c7ccccc76)cc5c34)n2)cc1]

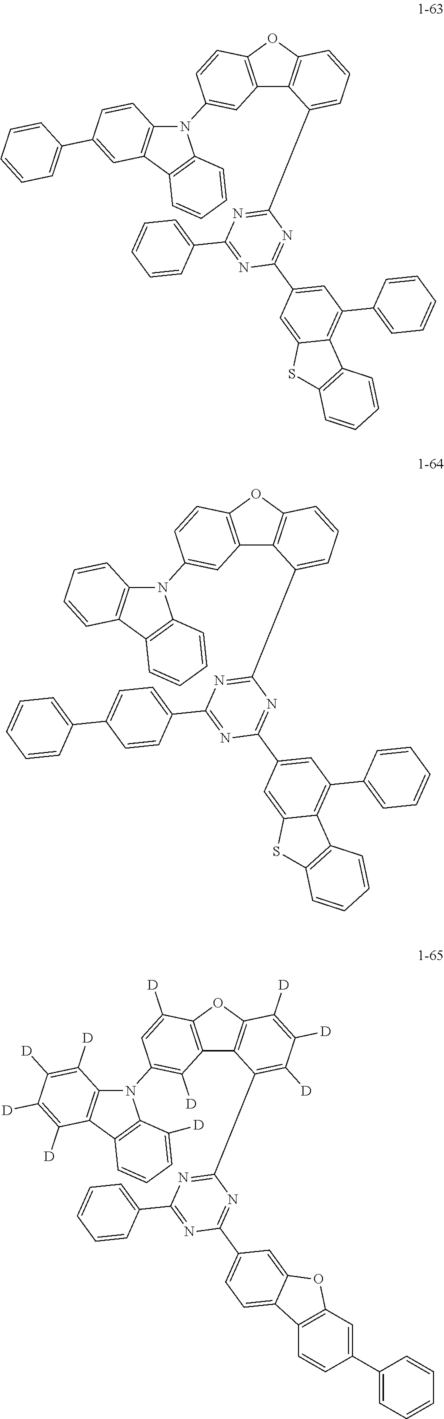 [SMILES: c1ccc(-c2ccc3c(c2)c2ccccc2n3-c2ccc3oc4cccc(-c5nc(-c6ccccc6)nc(-c6cc(-c7ccccc7)c7c(c6)sc6ccccc67)n5)c4c3c2)cc1 ; c1ccc(-c2ccc(-c3nc(-c4cc(-c5ccccc5)c5c(c4)sc4ccccc45)nc(-c4cccc5oc6ccc(-n7c8ccccc8c8ccccc87)cc6c45)n3)cc2)cc1 ; [2H]c1c([2H])c(-c2nc(-c3ccccc3)nc(-c3ccc4c(c3)oc3cc(-c5ccccc5)ccc34)n2)c2c(oc3c([2H])cc(-n4c5c([2H])cccc5c5c([2H])c([2H])c([2H])c([2H])c54)c([2H])c32)c1[2H]]

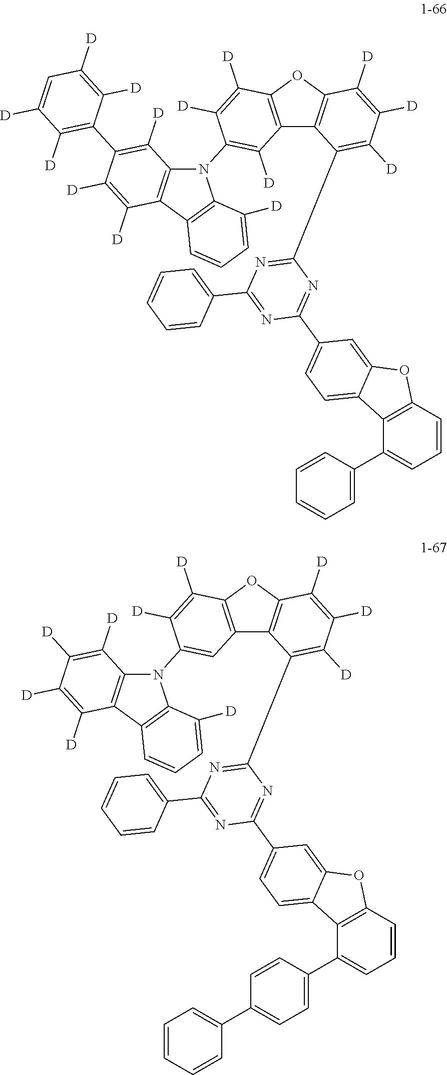

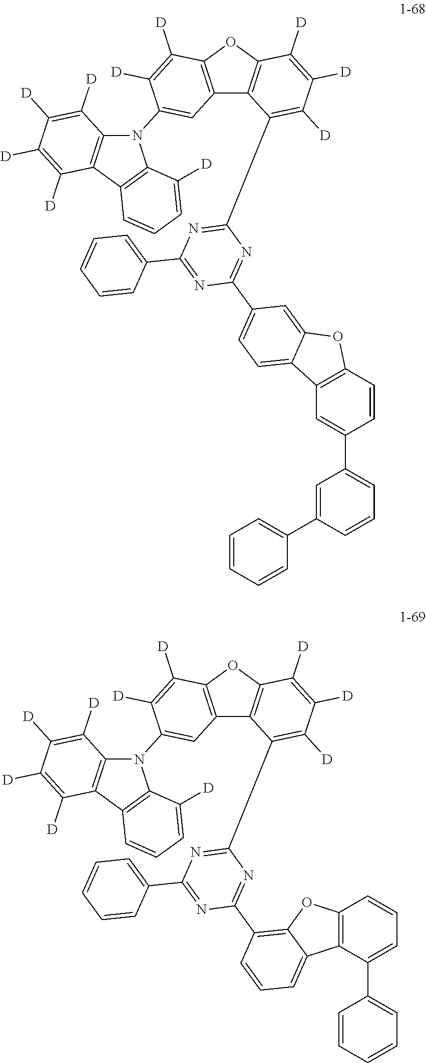 [SMILES: [2H]c1c(-n2c3c([2H])cccc3c3c([2H])c([2H])c([2H])c([2H])c32)cc2c(oc3c([2H])c([2H])c([2H])c(-c4nc(-c5ccccc5)nc(-c5ccc6c(c5)oc5ccc(-c7cccc(-c8ccccc8)c7)cc56)n4)c32)c1[2H] ; [2H]c1c(-n2c3c([2H])cccc3c3c([2H])c([2H])c([2H])c([2H])c32)cc2c(oc3c([2H])c([2H])c([2H])c(-c4nc(-c5ccccc5)nc(-c5cccc6c5oc5cccc(-c7ccccc7)c56)n4)c32)c1[2H]]

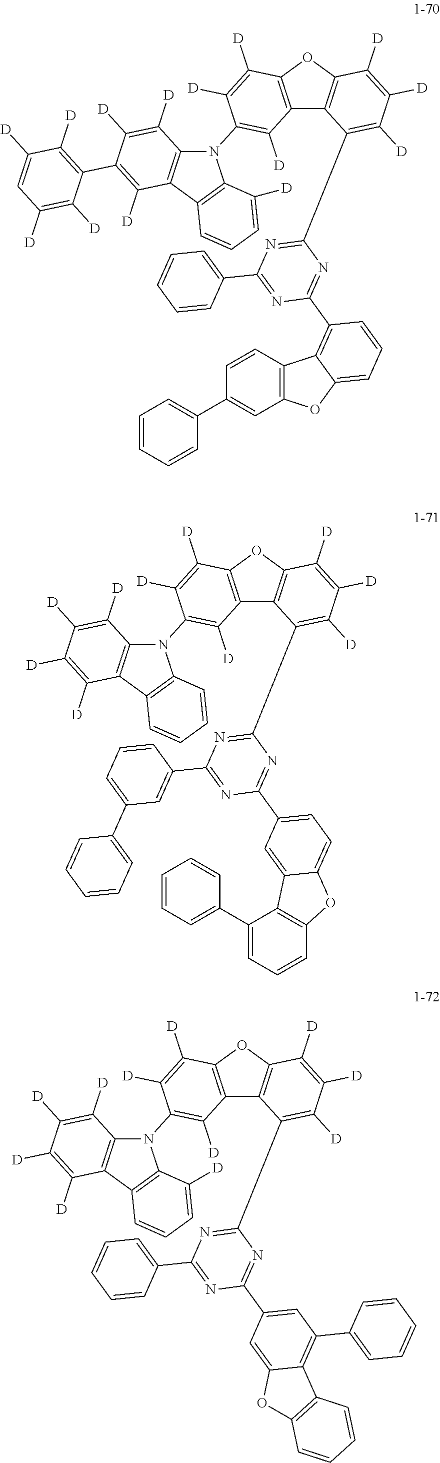

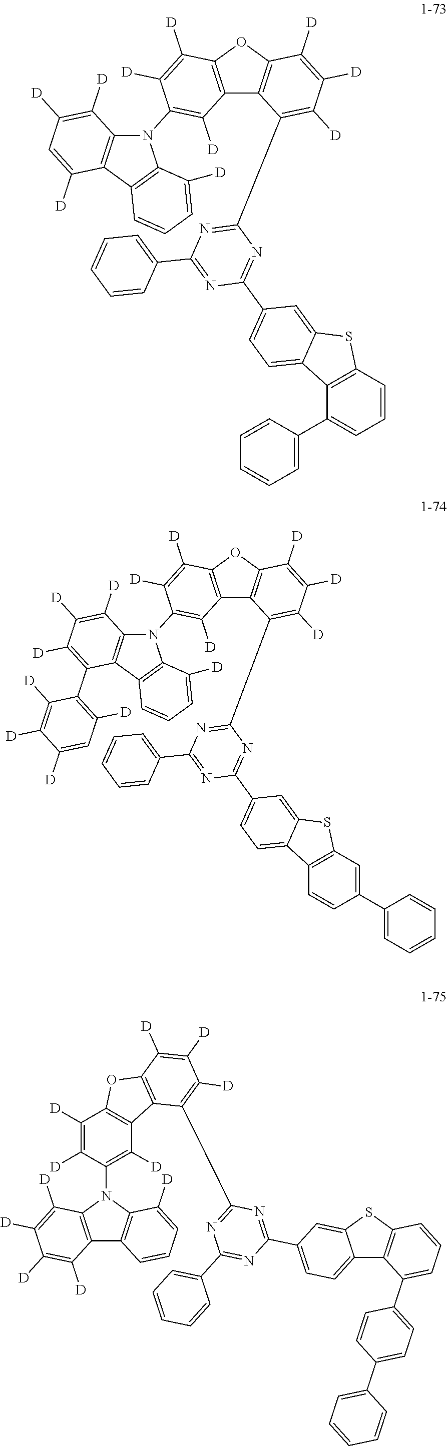

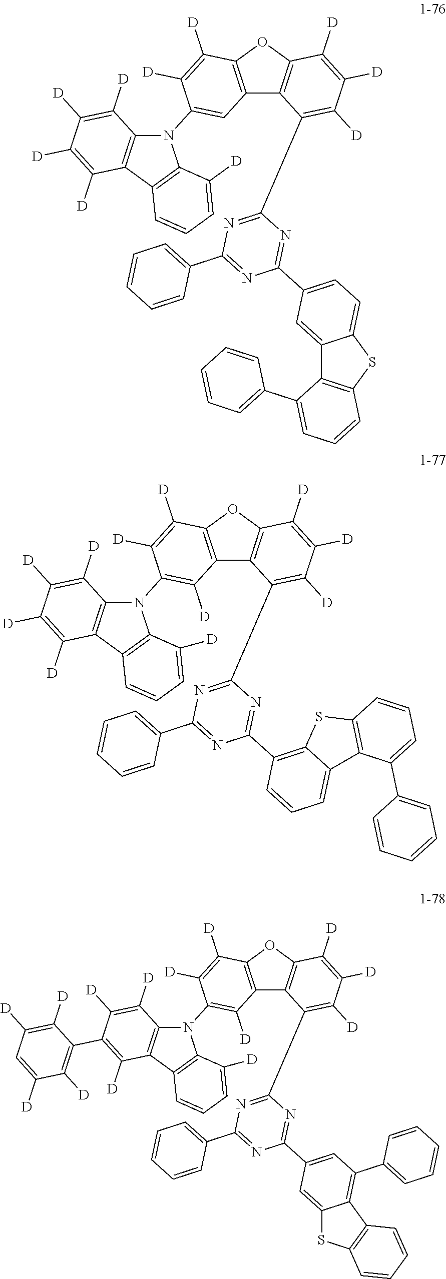

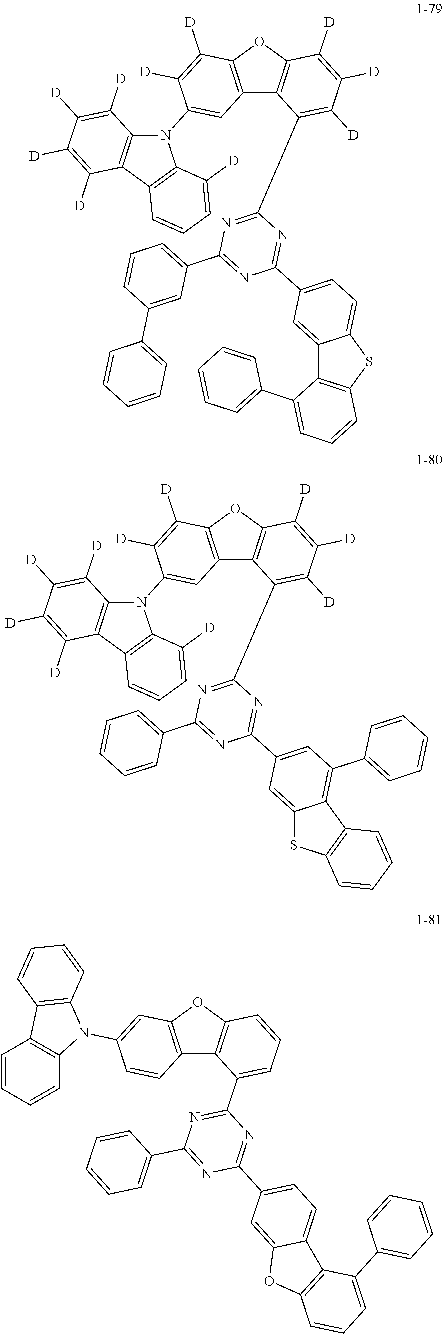 [SMILES: [2H]c1c(-n2c3c([2H])cccc3c3c([2H])c([2H])c([2H])c([2H])c32)cc2c(oc3c([2H])c([2H])c([2H])c(-c4nc(-c5cccc(-c6ccccc6)c5)nc(-c5ccc6sc7cccc(-c8ccccc8)c7c6c5)n4)c32)c1[2H] ; [2H]c1c(-n2c3c([2H])cccc3c3c([2H])c([2H])c([2H])c([2H])c32)cc2c(oc3c([2H])c([2H])c([2H])c(-c4nc(-c5ccccc5)nc(-c5cc(-c6ccccc6)c6c(c5)sc5ccccc56)n4)c32)c1[2H] ; c1ccc(-c2nc(-c3ccc4c(c3)oc3cccc(-c5ccccc5)c34)nc(-c3cccc4oc5cc(-n6c7ccccc7c7ccccc76)ccc5c34)n2)cc1]

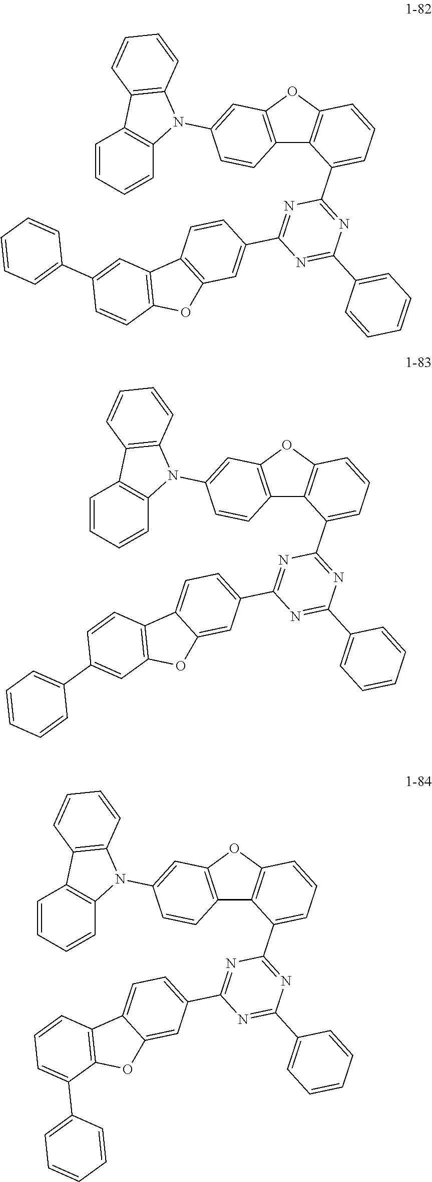 [SMILES: c1ccc(-c2ccc3oc4cc(-c5nc(-c6ccccc6)nc(-c6cccc7oc8cc(-n9c%10ccccc%10c%10ccccc%109)ccc8c67)n5)ccc4c3c2)cc1 ; c1ccc(-c2ccc3c(c2)oc2cc(-c4nc(-c5ccccc5)nc(-c5cccc6oc7cc(-n8c9ccccc9c9ccccc98)ccc7c56)n4)ccc23)cc1 ; c1ccc(-c2nc(-c3ccc4c(c3)oc3c(-c5ccccc5)cccc34)nc(-c3cccc4oc5cc(-n6c7ccccc7c7ccccc76)ccc5c34)n2)cc1]

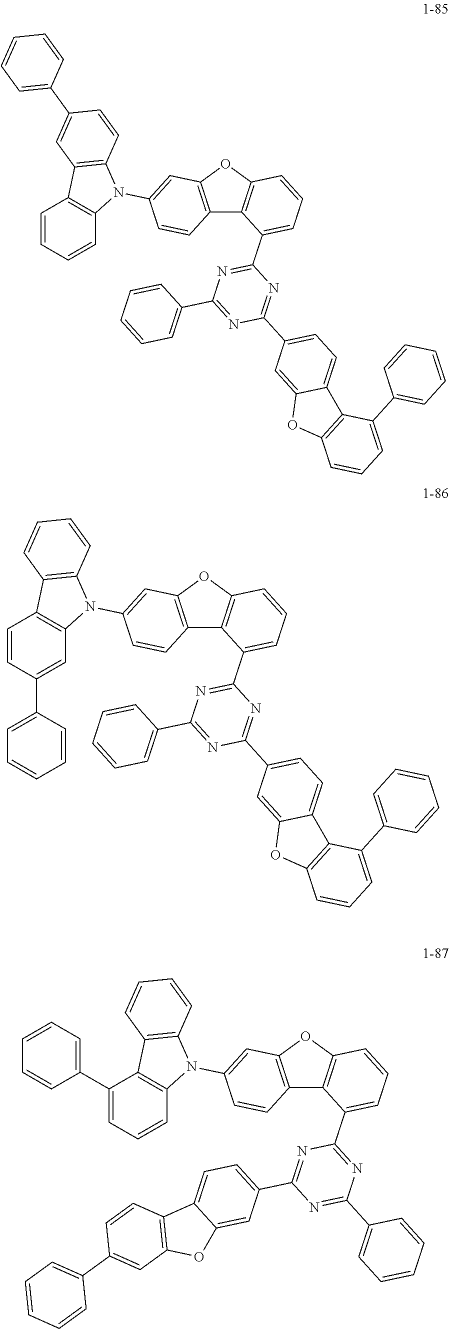 [SMILES: c1ccc(-c2ccc3c(c2)c2ccccc2n3-c2ccc3c(c2)oc2cccc(-c4nc(-c5ccccc5)nc(-c5ccc6c(c5)oc5cccc(-c7ccccc7)c56)n4)c23)cc1 ; c1ccc(-c2ccc3c4ccccc4n(-c4ccc5c(c4)oc4cccc(-c6nc(-c7ccccc7)nc(-c7ccc8c(c7)oc7cccc(-c9ccccc9)c78)n6)c45)c3c2)cc1 ; c1ccc(-c2ccc3c(c2)oc2cc(-c4nc(-c5ccccc5)nc(-c5cccc6oc7cc(-n8c9ccccc9c9c(-c%10ccccc%10)cccc98)ccc7c56)n4)ccc23)cc1]

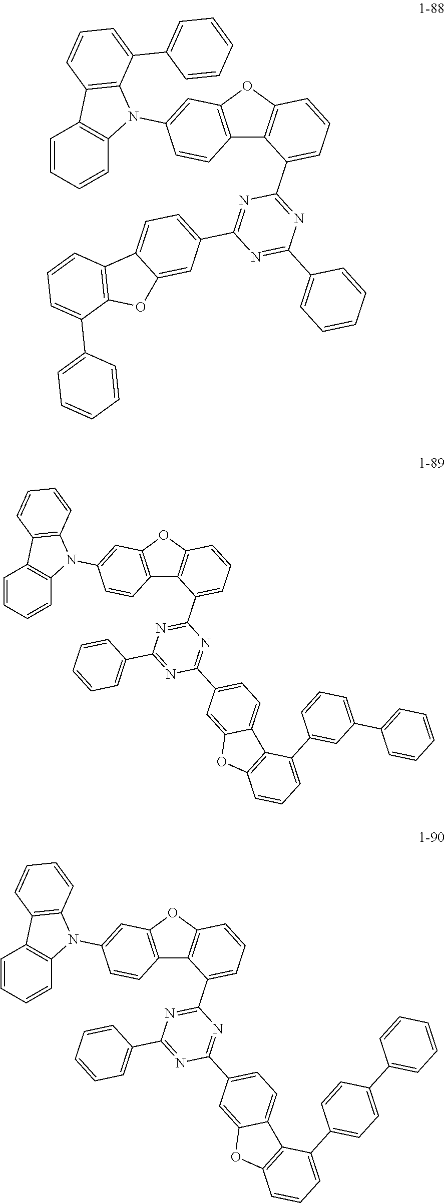 [SMILES: c1ccc(-c2nc(-c3ccc4c(c3)oc3c(-c5ccccc5)cccc34)nc(-c3cccc4oc5cc(-n6c7ccccc7c7cccc(-c8ccccc8)c76)ccc5c34)n2)cc1 ; c1ccc(-c2cccc(-c3cccc4oc5cc(-c6nc(-c7ccccc7)nc(-c7cccc8oc9cc(-n%10c%11ccccc%11c%11ccccc%11%10)ccc9c78)n6)ccc5c34)c2)cc1 ; c1ccc(-c2ccc(-c3cccc4oc5cc(-c6nc(-c7ccccc7)nc(-c7cccc8oc9cc(-n%10c%11ccccc%11c%11ccccc%11%10)ccc9c78)n6)ccc5c34)cc2)cc1]

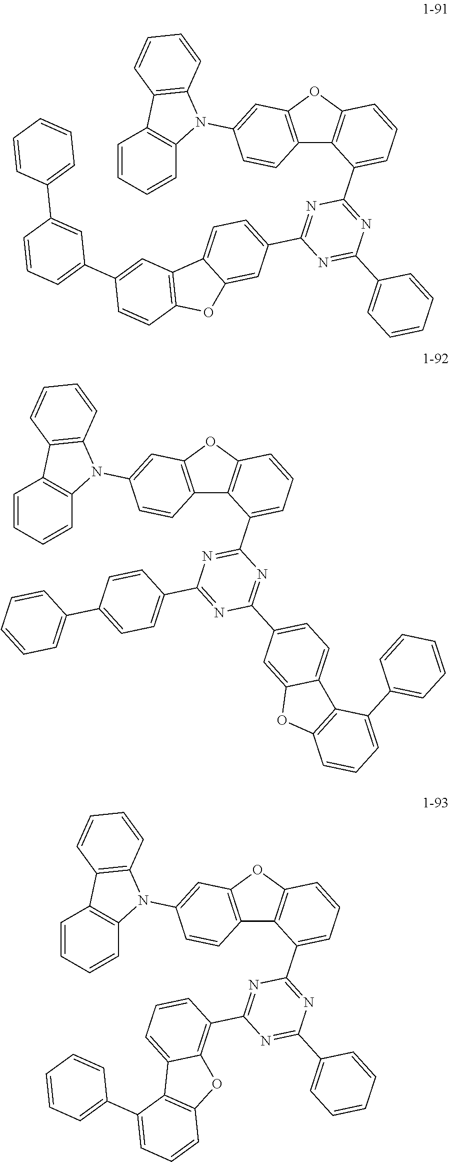 [SMILES: c1ccc(-c2cccc(-c3ccc4oc5cc(-c6nc(-c7ccccc7)nc(-c7cccc8oc9cc(-n%10c%11ccccc%11c%11ccccc%11%10)ccc9c78)n6)ccc5c4c3)c2)cc1 ; c1ccc(-c2ccc(-c3nc(-c4ccc5c(c4)oc4cccc(-c6ccccc6)c45)nc(-c4cccc5oc6cc(-n7c8ccccc8c8ccccc87)ccc6c45)n3)cc2)cc1 ; c1ccc(-c2nc(-c3cccc4c3oc3cccc(-c5ccccc5)c34)nc(-c3cccc4oc5cc(-n6c7ccccc7c7ccccc76)ccc5c34)n2)cc1]

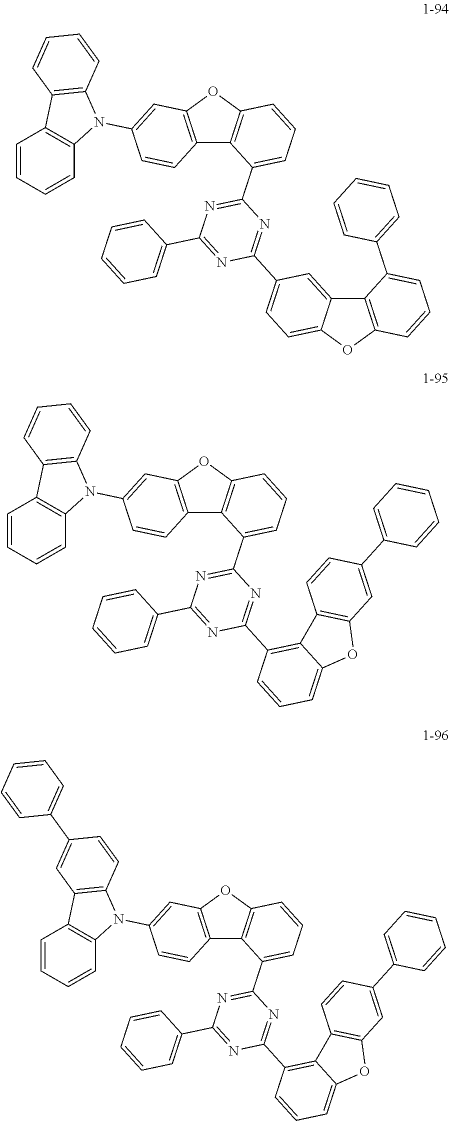 [SMILES: c1ccc(-c2nc(-c3ccc4oc5cccc(-c6ccccc6)c5c4c3)nc(-c3cccc4oc5cc(-n6c7ccccc7c7ccccc76)ccc5c34)n2)cc1 ; c1ccc(-c2ccc3c(c2)oc2cccc(-c4nc(-c5ccccc5)nc(-c5cccc6oc7cc(-n8c9ccccc9c9ccccc98)ccc7c56)n4)c23)cc1 ; c1ccc(-c2ccc3c(c2)oc2cccc(-c4nc(-c5ccccc5)nc(-c5cccc6oc7cc(-n8c9ccccc9c9cc(-c%10ccccc%10)ccc98)ccc7c56)n4)c23)cc1]

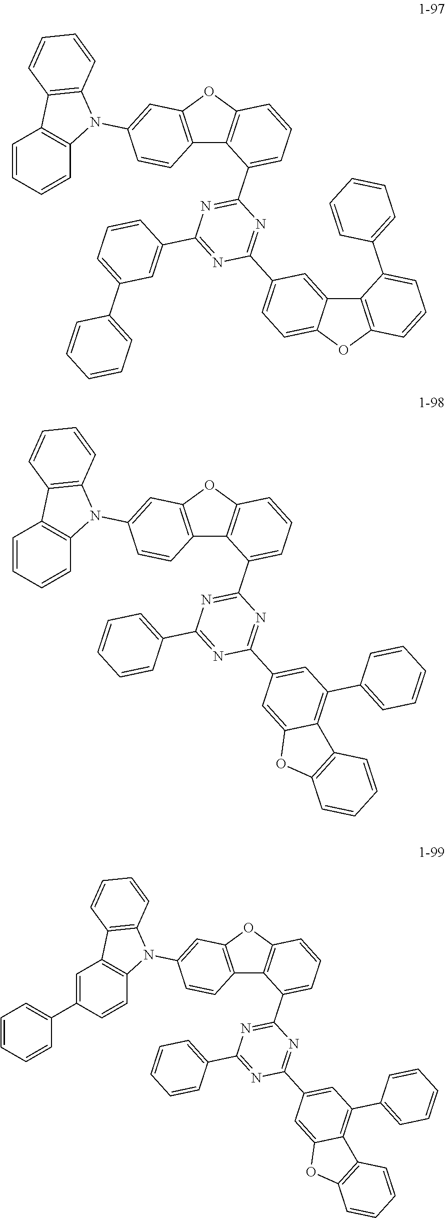 [SMILES: c1ccc(-c2cccc(-c3nc(-c4ccc5oc6cccc(-c7ccccc7)c6c5c4)nc(-c4cccc5oc6cc(-n7c8ccccc8c8ccccc87)ccc6c45)n3)c2)cc1 ; c1ccc(-c2nc(-c3cc(-c4ccccc4)c4c(c3)oc3ccccc34)nc(-c3cccc4oc5cc(-n6c7ccccc7c7ccccc76)ccc5c34)n2)cc1 ; c1ccc(-c2ccc3c(c2)c2ccccc2n3-c2ccc3c(c2)oc2cccc(-c4nc(-c5ccccc5)nc(-c5cc(-c6ccccc6)c6c(c5)oc5ccccc56)n4)c23)cc1]

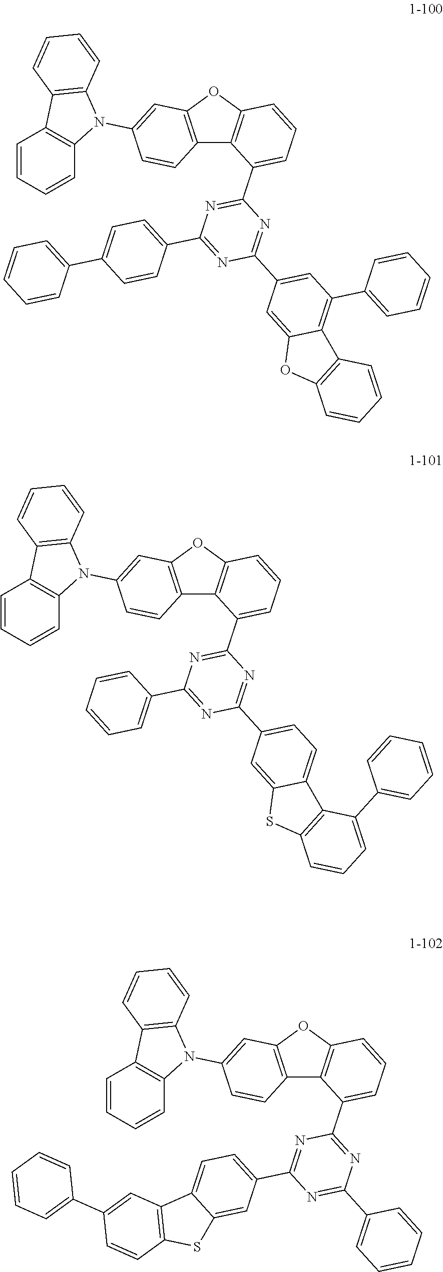 [SMILES: c1ccc(-c2ccc(-c3nc(-c4cc(-c5ccccc5)c5c(c4)oc4ccccc45)nc(-c4cccc5oc6cc(-n7c8ccccc8c8ccccc87)ccc6c45)n3)cc2)cc1 ; c1ccc(-c2nc(-c3ccc4c(c3)sc3cccc(-c5ccccc5)c34)nc(-c3cccc4oc5cc(-n6c7ccccc7c7ccccc76)ccc5c34)n2)cc1 ; c1ccc(-c2ccc3sc4cc(-c5nc(-c6ccccc6)nc(-c6cccc7oc8cc(-n9c%10ccccc%10c%10ccccc%109)ccc8c67)n5)ccc4c3c2)cc1]

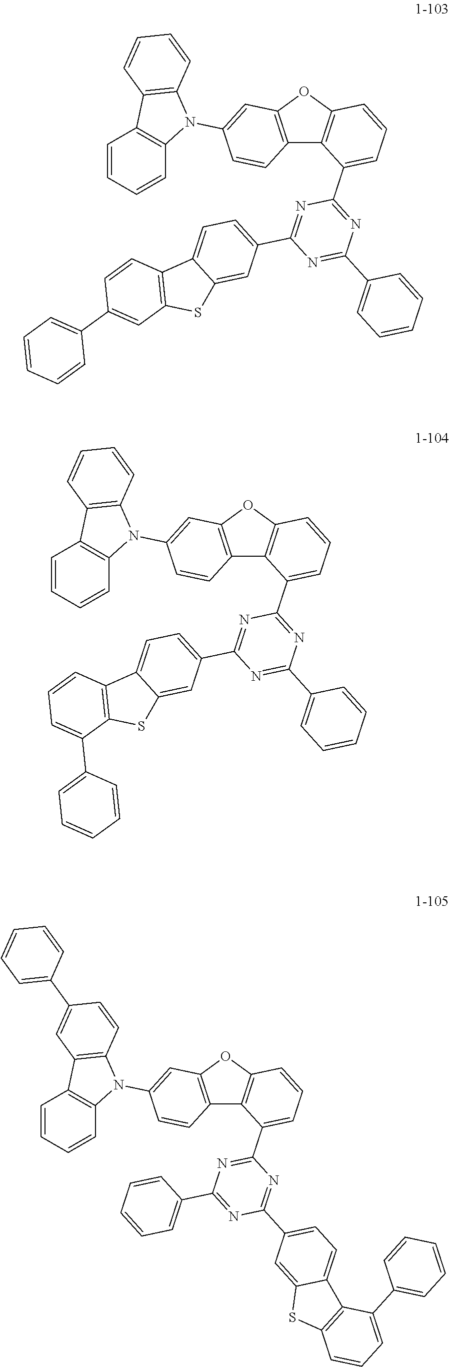 [SMILES: c1ccc(-c2ccc3c(c2)sc2cc(-c4nc(-c5ccccc5)nc(-c5cccc6oc7cc(-n8c9ccccc9c9ccccc98)ccc7c56)n4)ccc23)cc1 ; c1ccc(-c2nc(-c3ccc4c(c3)sc3c(-c5ccccc5)cccc34)nc(-c3cccc4oc5cc(-n6c7ccccc7c7ccccc76)ccc5c34)n2)cc1 ; c1ccc(-c2ccc3c(c2)c2ccccc2n3-c2ccc3c(c2)oc2cccc(-c4nc(-c5ccccc5)nc(-c5ccc6c(c5)sc5cccc(-c7ccccc7)c56)n4)c23)cc1]

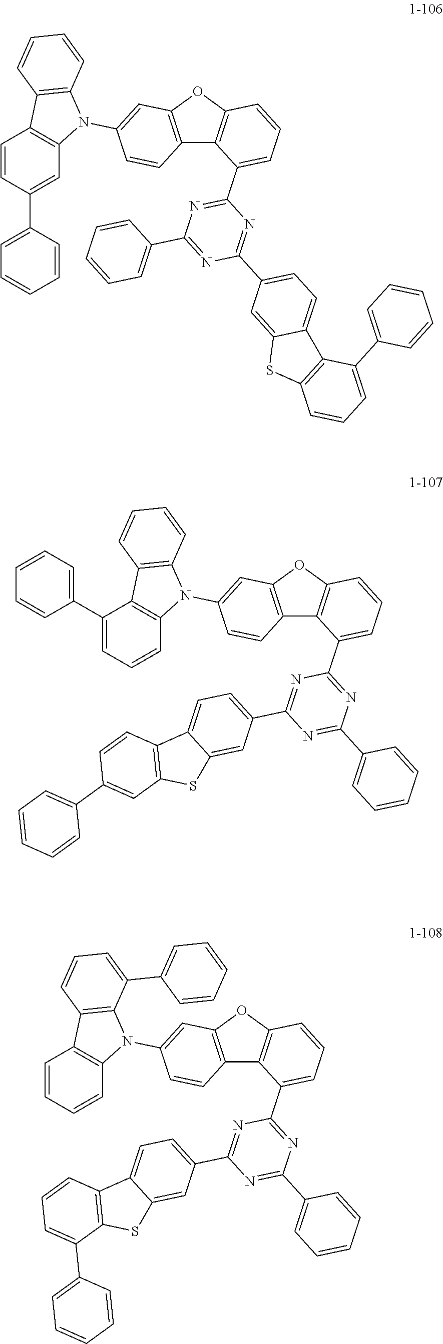 [SMILES: c1ccc(-c2ccc3c4ccccc4n(-c4ccc5c(c4)oc4cccc(-c6nc(-c7ccccc7)nc(-c7ccc8c(c7)sc7cccc(-c9ccccc9)c78)n6)c45)c3c2)cc1 ; c1ccc(-c2ccc3c(c2)sc2cc(-c4nc(-c5ccccc5)nc(-c5cccc6oc7cc(-n8c9ccccc9c9c(-c%10ccccc%10)cccc98)ccc7c56)n4)ccc23)cc1 ; c1ccc(-c2nc(-c3ccc4c(c3)sc3c(-c5ccccc5)cccc34)nc(-c3cccc4oc5cc(-n6c7ccccc7c7cccc(-c8ccccc8)c76)ccc5c34)n2)cc1]

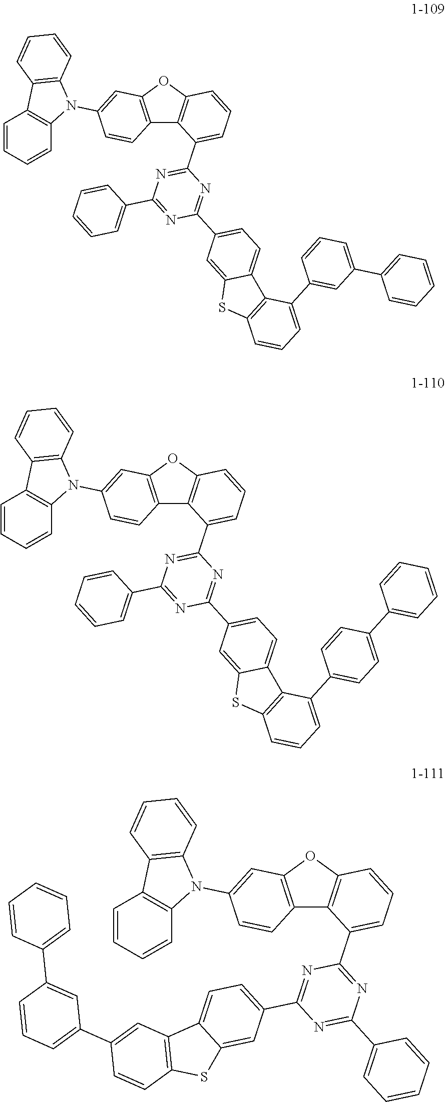 [SMILES: c1ccc(-c2cccc(-c3cccc4sc5cc(-c6nc(-c7ccccc7)nc(-c7cccc8oc9cc(-n%10c%11ccccc%11c%11ccccc%11%10)ccc9c78)n6)ccc5c34)c2)cc1 ; c1ccc(-c2ccc(-c3cccc4sc5cc(-c6nc(-c7ccccc7)nc(-c7cccc8oc9cc(-n%10c%11ccccc%11c%11ccccc%11%10)ccc9c78)n6)ccc5c34)cc2)cc1 ; c1ccc(-c2cccc(-c3ccc4sc5cc(-c6nc(-c7ccccc7)nc(-c7cccc8oc9cc(-n%10c%11ccccc%11c%11ccccc%11%10)ccc9c78)n6)ccc5c4c3)c2)cc1]

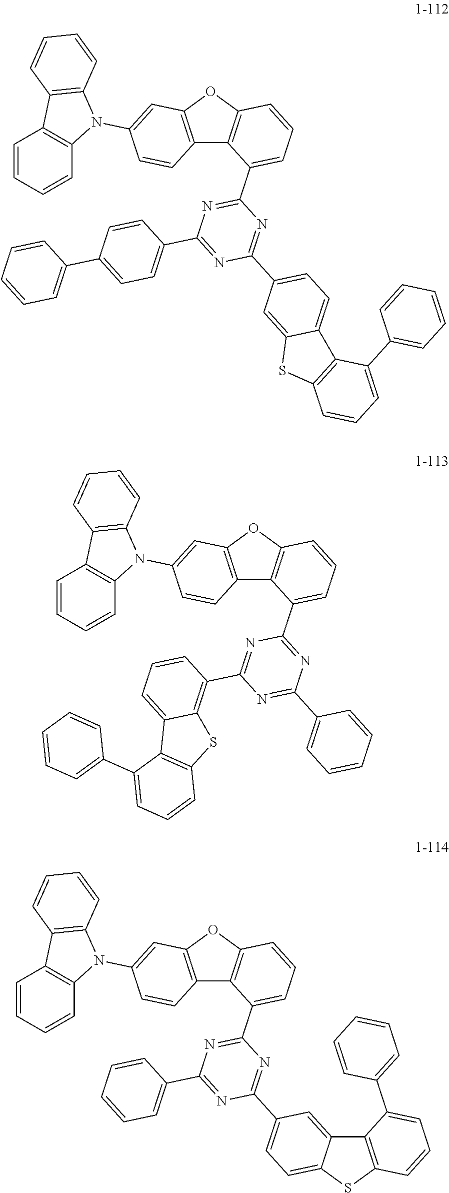 [SMILES: c1ccc(-c2ccc(-c3nc(-c4ccc5c(c4)sc4cccc(-c6ccccc6)c45)nc(-c4cccc5oc6cc(-n7c8ccccc8c8ccccc87)ccc6c45)n3)cc2)cc1 ; c1ccc(-c2nc(-c3cccc4c3sc3cccc(-c5ccccc5)c34)nc(-c3cccc4oc5cc(-n6c7ccccc7c7ccccc76)ccc5c34)n2)cc1 ; c1ccc(-c2nc(-c3ccc4sc5cccc(-c6ccccc6)c5c4c3)nc(-c3cccc4oc5cc(-n6c7ccccc7c7ccccc76)ccc5c34)n2)cc1]

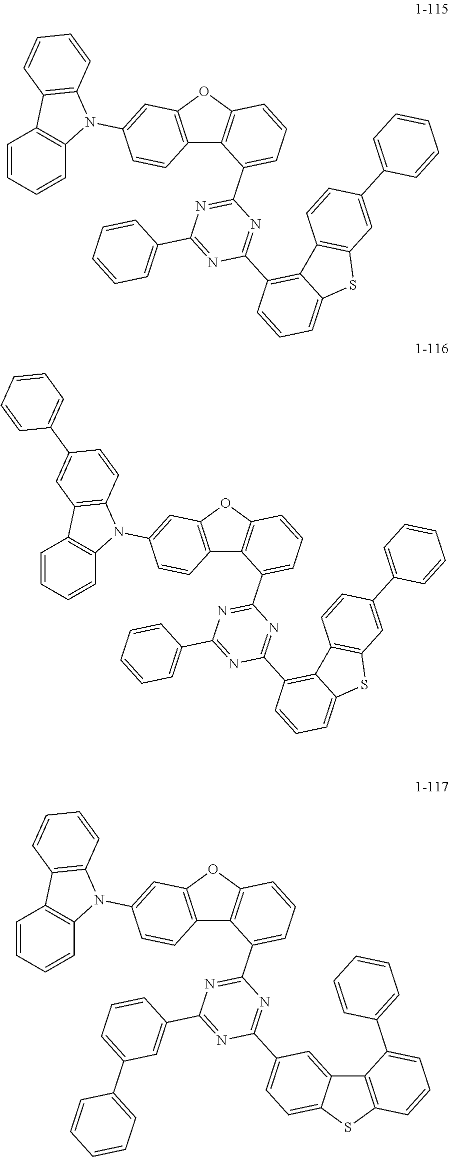 [SMILES: c1ccc(-c2ccc3c(c2)sc2cccc(-c4nc(-c5ccccc5)nc(-c5cccc6oc7cc(-n8c9ccccc9c9ccccc98)ccc7c56)n4)c23)cc1 ; c1ccc(-c2ccc3c(c2)sc2cccc(-c4nc(-c5ccccc5)nc(-c5cccc6oc7cc(-n8c9ccccc9c9cc(-c%10ccccc%10)ccc98)ccc7c56)n4)c23)cc1 ; c1ccc(-c2cccc(-c3nc(-c4ccc5sc6cccc(-c7ccccc7)c6c5c4)nc(-c4cccc5oc6cc(-n7c8ccccc8c8ccccc87)ccc6c45)n3)c2)cc1]

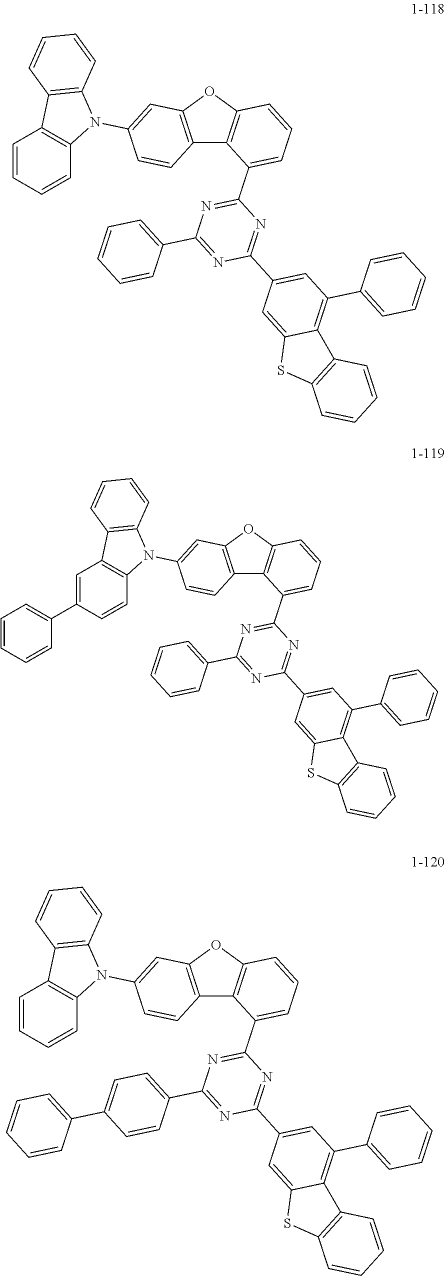 [SMILES: c1ccc(-c2nc(-c3cc(-c4ccccc4)c4c(c3)sc3ccccc34)nc(-c3cccc4oc5cc(-n6c7ccccc7c7ccccc76)ccc5c34)n2)cc1 ; c1ccc(-c2ccc3c(c2)c2ccccc2n3-c2ccc3c(c2)oc2cccc(-c4nc(-c5ccccc5)nc(-c5cc(-c6ccccc6)c6c(c5)sc5ccccc56)n4)c23)cc1 ; c1ccc(-c2ccc(-c3nc(-c4cc(-c5ccccc5)c5c(c4)sc4ccccc45)nc(-c4cccc5oc6cc(-n7c8ccccc8c8ccccc87)ccc6c45)n3)cc2)cc1]

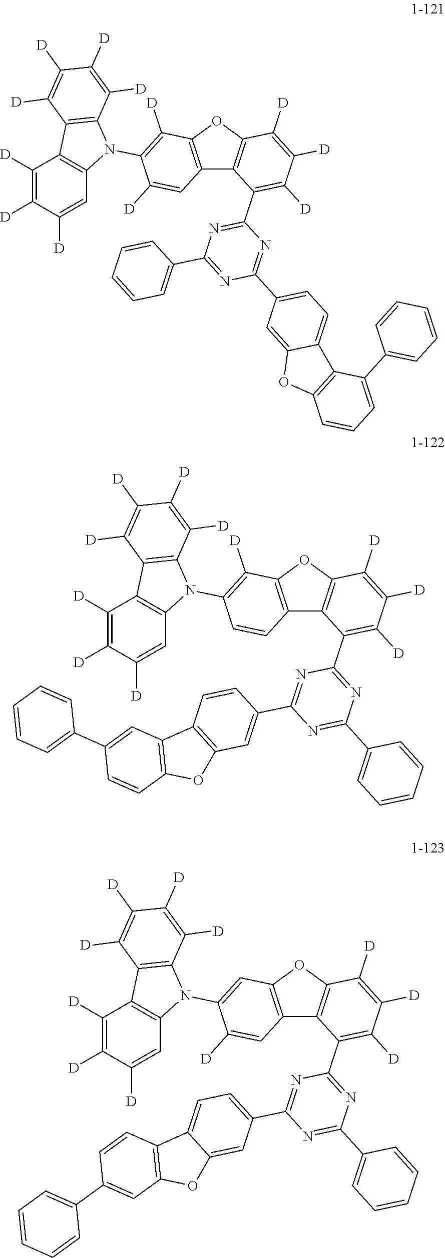 [SMILES: [2H]c1cc2c(c([2H])c1[2H])c1c([2H])c([2H])c([2H])c([2H])c1n2-c1c([2H])cc2c(oc3c([2H])c([2H])c([2H])c(-c4nc(-c5ccccc5)nc(-c5ccc6c(c5)oc5cccc(-c7ccccc7)c56)n4)c32)c1[2H] ; [2H]c1cc2c(c([2H])c1[2H])c1c([2H])c([2H])c([2H])c([2H])c1n2-c1ccc2c(oc3c([2H])c([2H])c([2H])c(-c4nc(-c5ccccc5)nc(-c5ccc6c(c5)oc5ccc(-c7ccccc7)cc56)n4)c32)c1[2H] ; [2H]c1cc2c(cc1-n1c3cc([2H])c([2H])c([2H])c3c3c([2H])c([2H])c([2H])c([2H])c31)oc1c([2H])c([2H])c([2H])c(-c3nc(-c4ccccc4)nc(-c4ccc5c(c4)oc4cc(-c6ccccc6)ccc45)n3)c12]

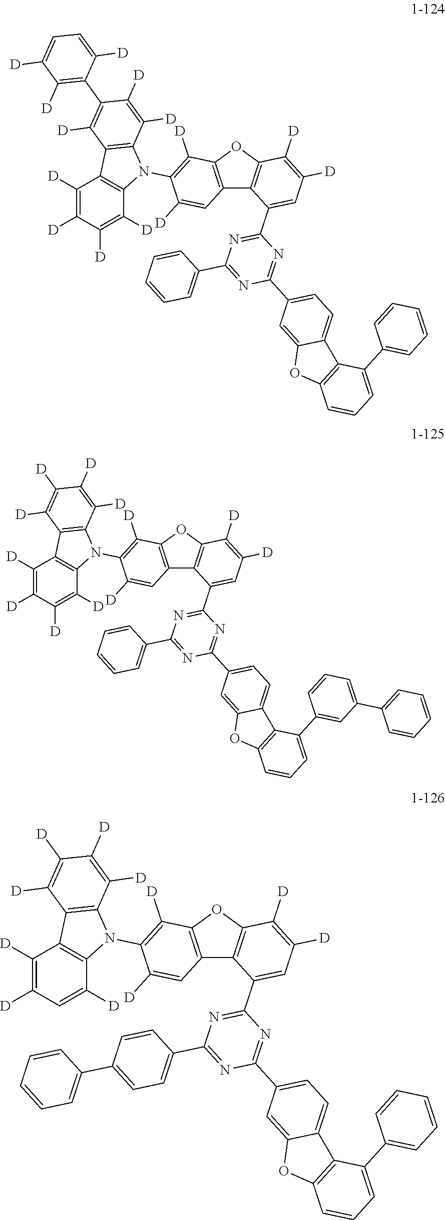

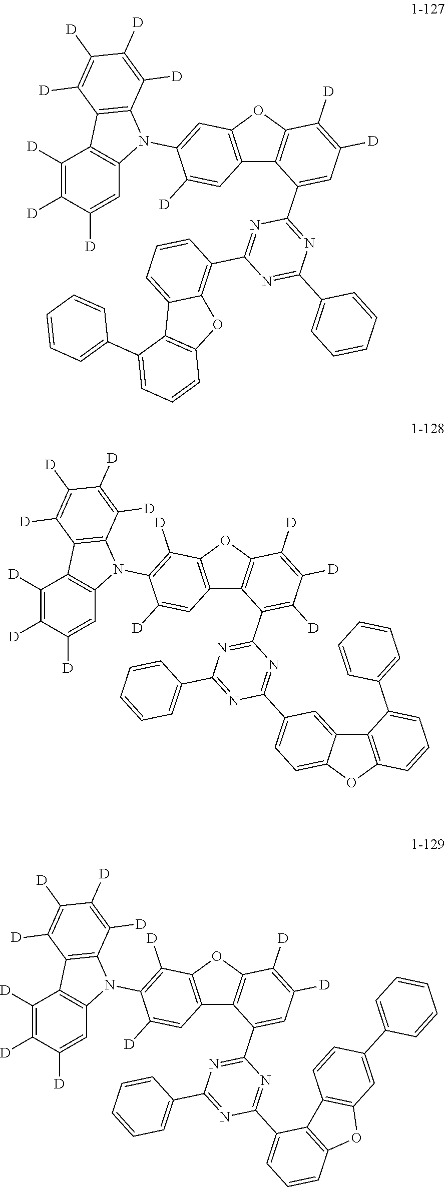 [SMILES: [2H]c1cc2c(cc1-n1c3cc([2H])c([2H])c([2H])c3c3c([2H])c([2H])c([2H])c([2H])c31)oc1c([2H])c([2H])cc(-c3nc(-c4ccccc4)nc(-c4cccc5c4oc4cccc(-c6ccccc6)c45)n3)c12 ; [2H]c1cc2c(c([2H])c1[2H])c1c([2H])c([2H])c([2H])c([2H])c1n2-c1c([2H])cc2c(oc3c([2H])c([2H])c([2H])c(-c4nc(-c5ccccc5)nc(-c5ccc6oc7cccc(-c8ccccc8)c7c6c5)n4)c32)c1[2H] ; [2H]c1cc2c(c([2H])c1[2H])c1c([2H])c([2H])c([2H])c([2H])c1n2-c1c([2H])cc2c(oc3c([2H])c([2H])cc(-c4nc(-c5ccccc5)nc(-c5cccc6oc7cc(-c8ccccc8)ccc7c56)n4)c32)c1[2H]]

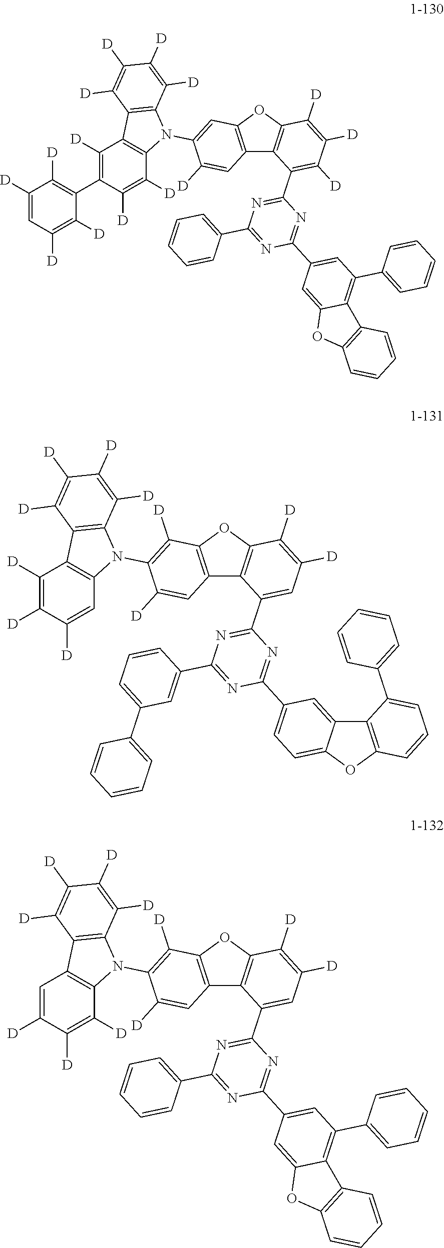

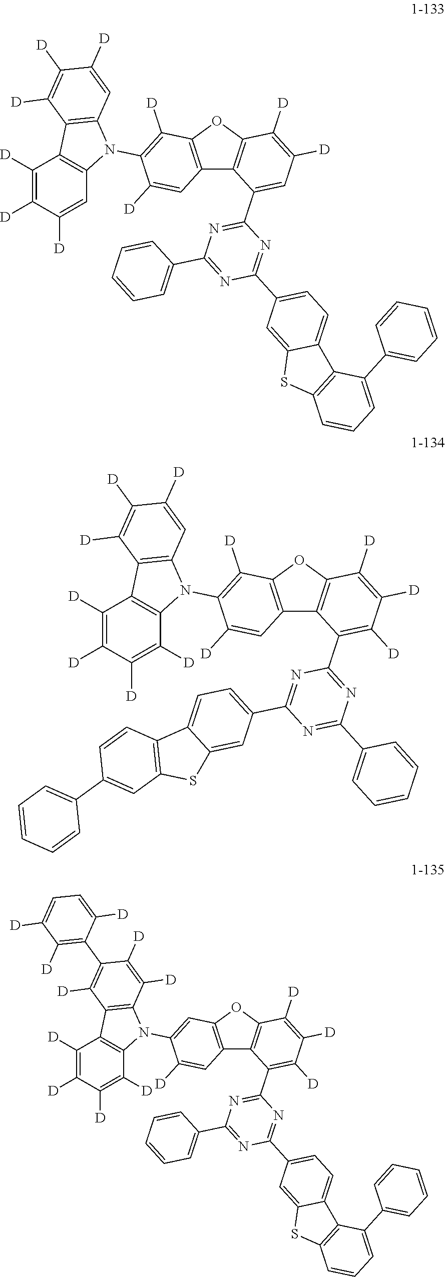

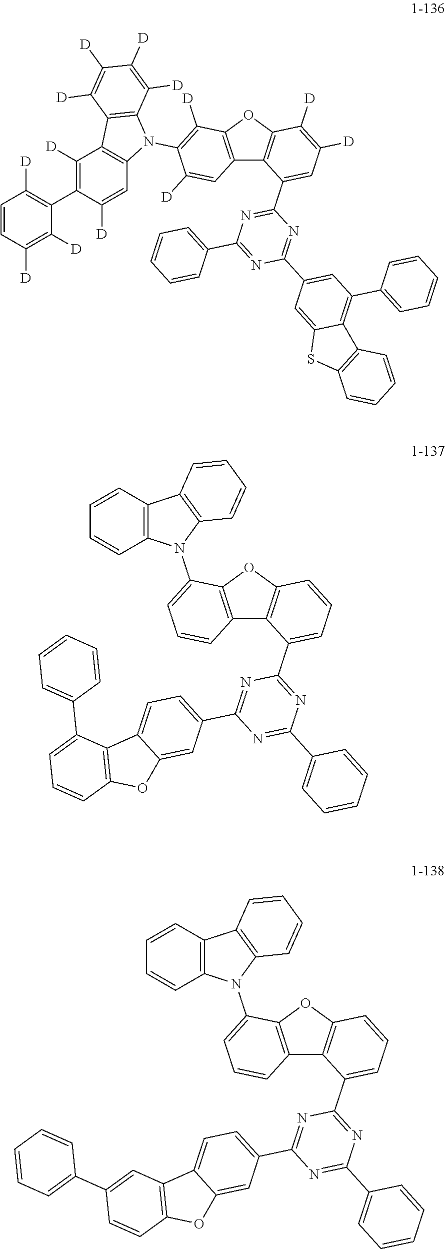 [SMILES: [2H]c1ccc([2H])c(-c2c([2H])cc3c(c2[2H])c2c([2H])c([2H])c([2H])c([2H])c2n3-c2c([2H])cc3c(oc4c([2H])c([2H])cc(-c5nc(-c6ccccc6)nc(-c6cc(-c7ccccc7)c7c(c6)sc6ccccc67)n5)c43)c2[2H])c1[2H] ; c1ccc(-c2nc(-c3ccc4c(c3)oc3cccc(-c5ccccc5)c34)nc(-c3cccc4oc5c(-n6c7ccccc7c7ccccc76)cccc5c34)n2)cc1 ; c1ccc(-c2ccc3oc4cc(-c5nc(-c6ccccc6)nc(-c6cccc7oc8c(-n9c%10ccccc%10c%10ccccc%109)cccc8c67)n5)ccc4c3c2)cc1]

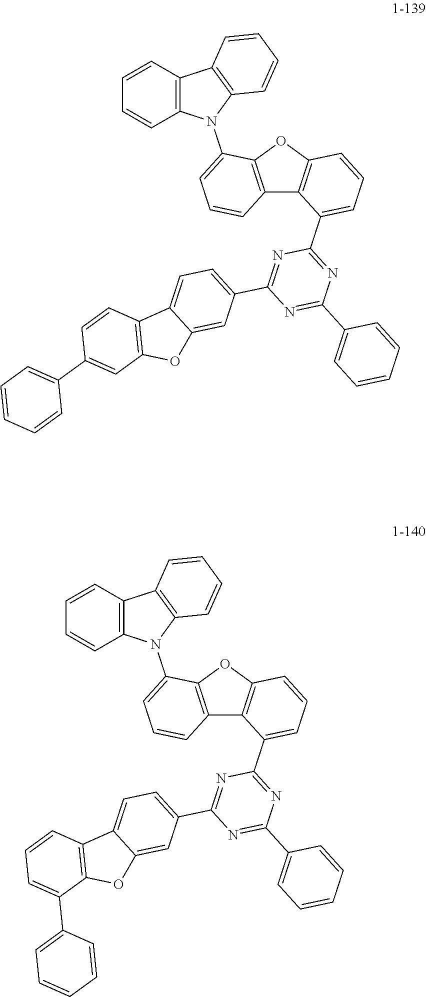 [SMILES: c1ccc(-c2ccc3c(c2)oc2cc(-c4nc(-c5ccccc5)nc(-c5cccc6oc7c(-n8c9ccccc9c9ccccc98)cccc7c56)n4)ccc23)cc1 ; c1ccc(-c2nc(-c3ccc4c(c3)oc3c(-c5ccccc5)cccc34)nc(-c3cccc4oc5c(-n6c7ccccc7c7ccccc76)cccc5c34)n2)cc1]

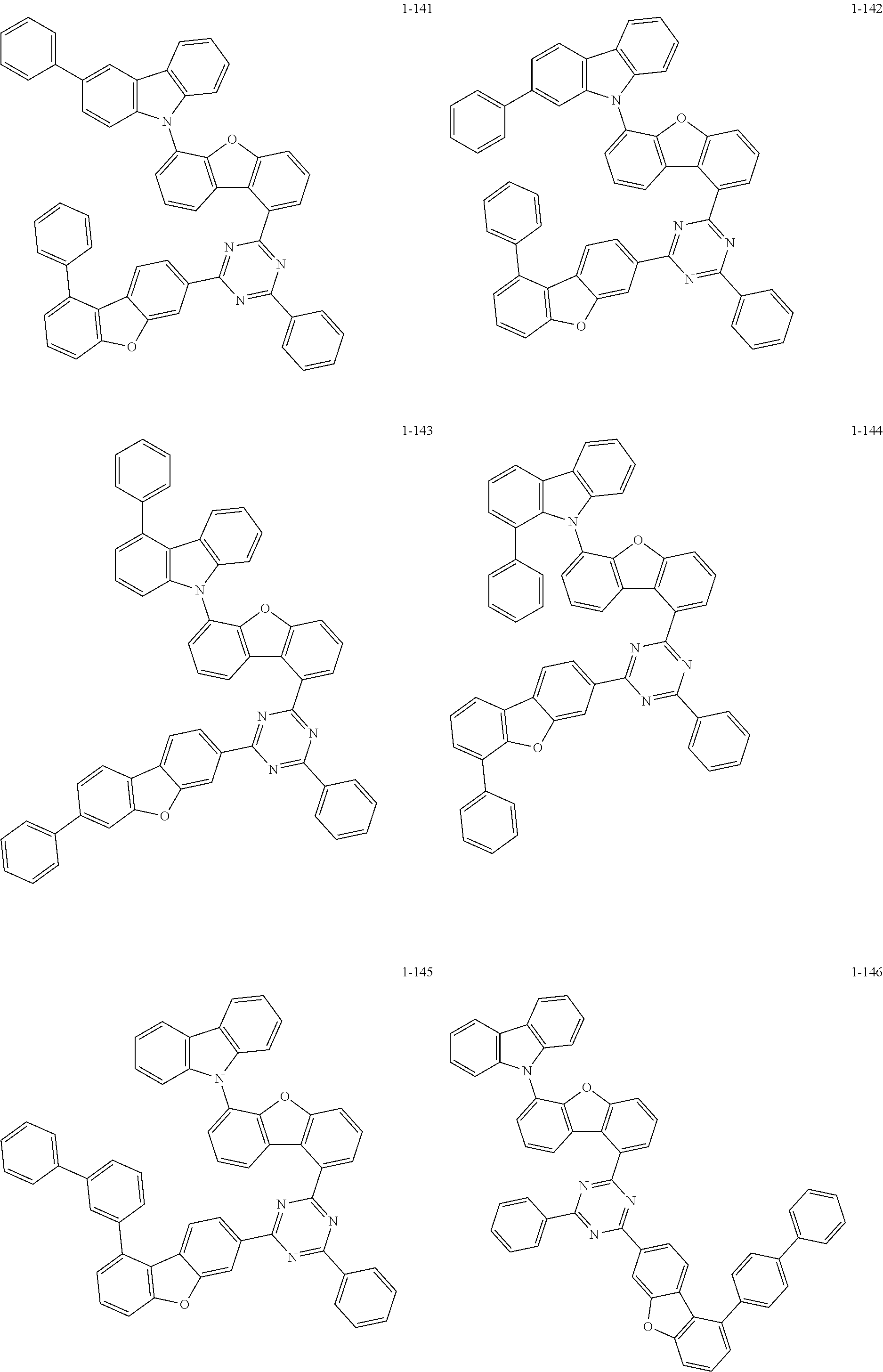

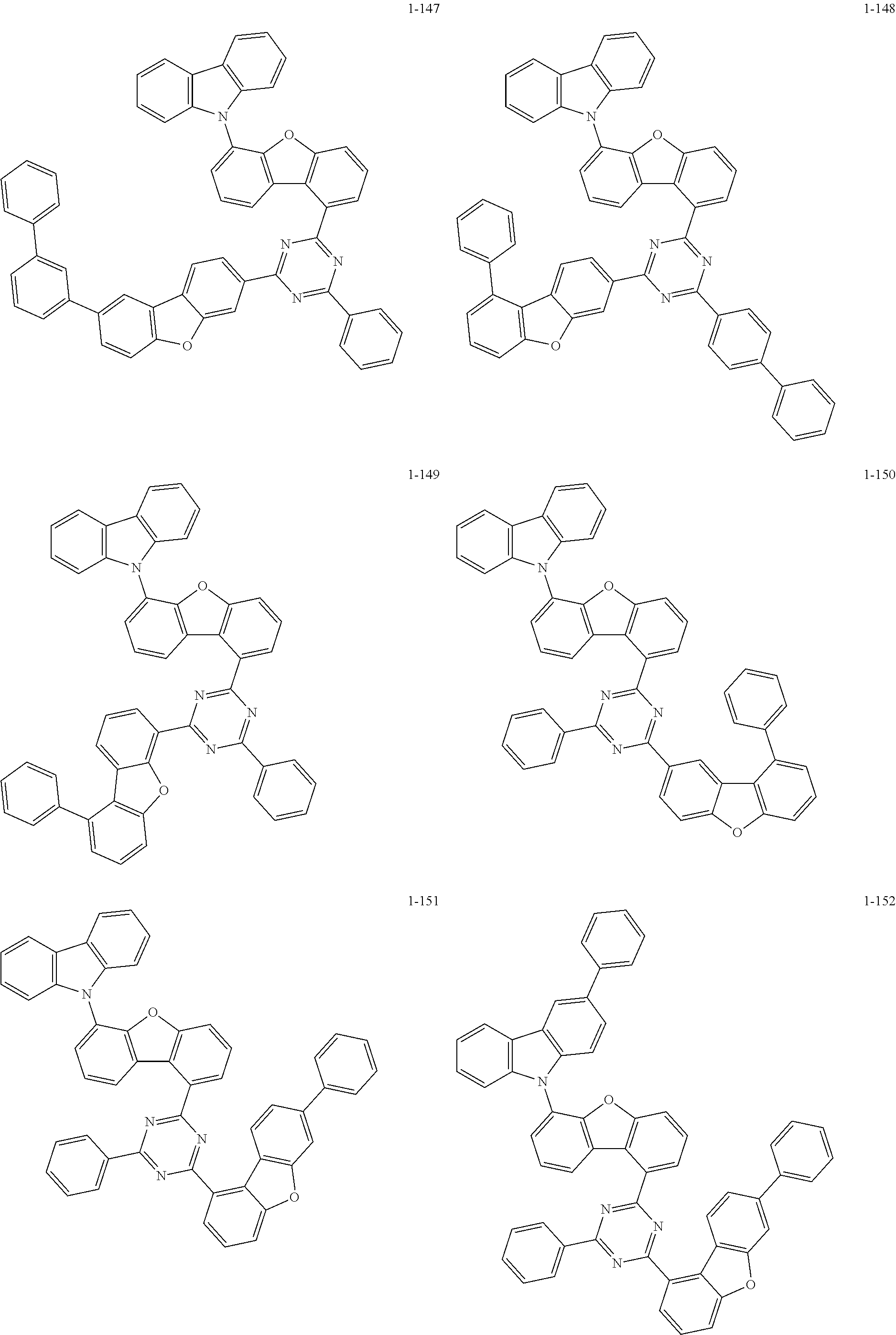

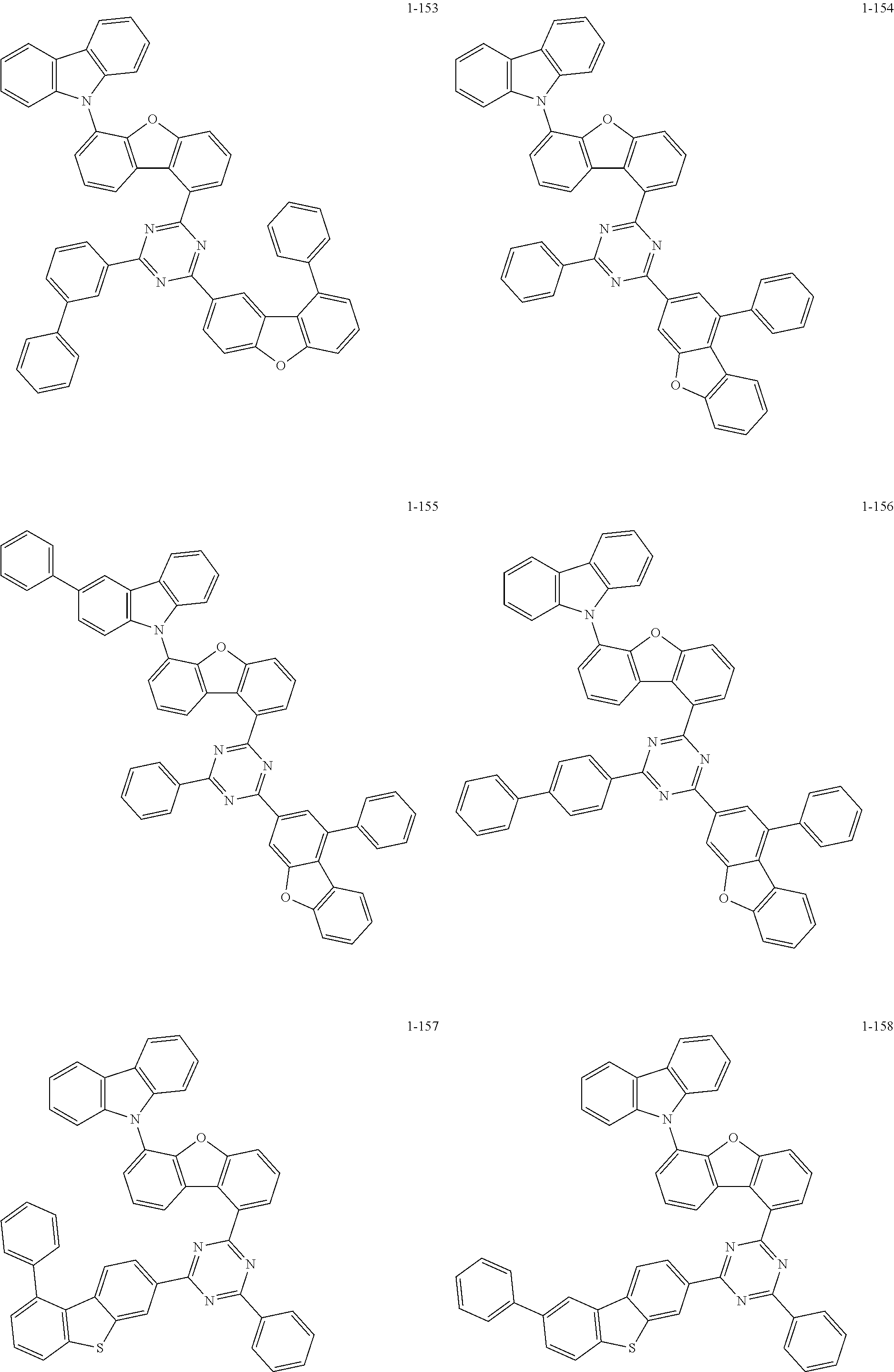

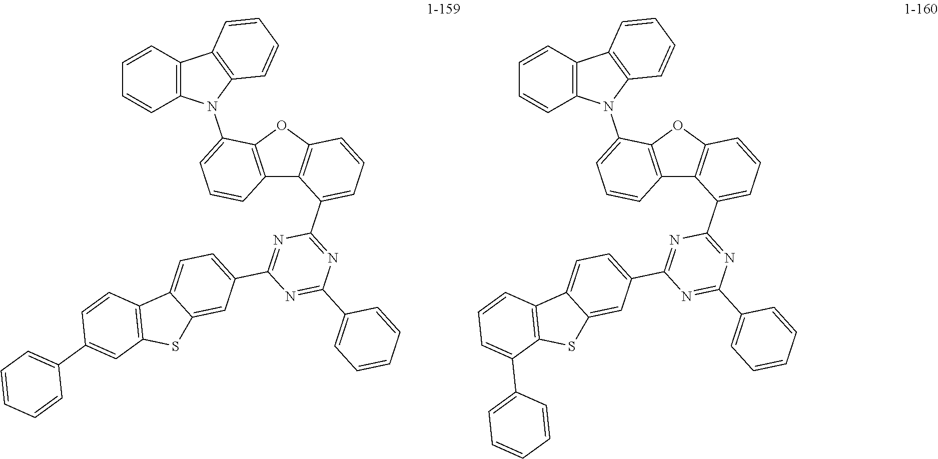 [SMILES: c1ccc(-c2ccc3c(c2)sc2cc(-c4nc(-c5ccccc5)nc(-c5cccc6oc7c(-n8c9ccccc9c9ccccc98)cccc7c56)n4)ccc23)cc1 ; c1ccc(-c2nc(-c3ccc4c(c3)sc3c(-c5ccccc5)cccc34)nc(-c3cccc4oc5c(-n6c7ccccc7c7ccccc76)cccc5c34)n2)cc1]

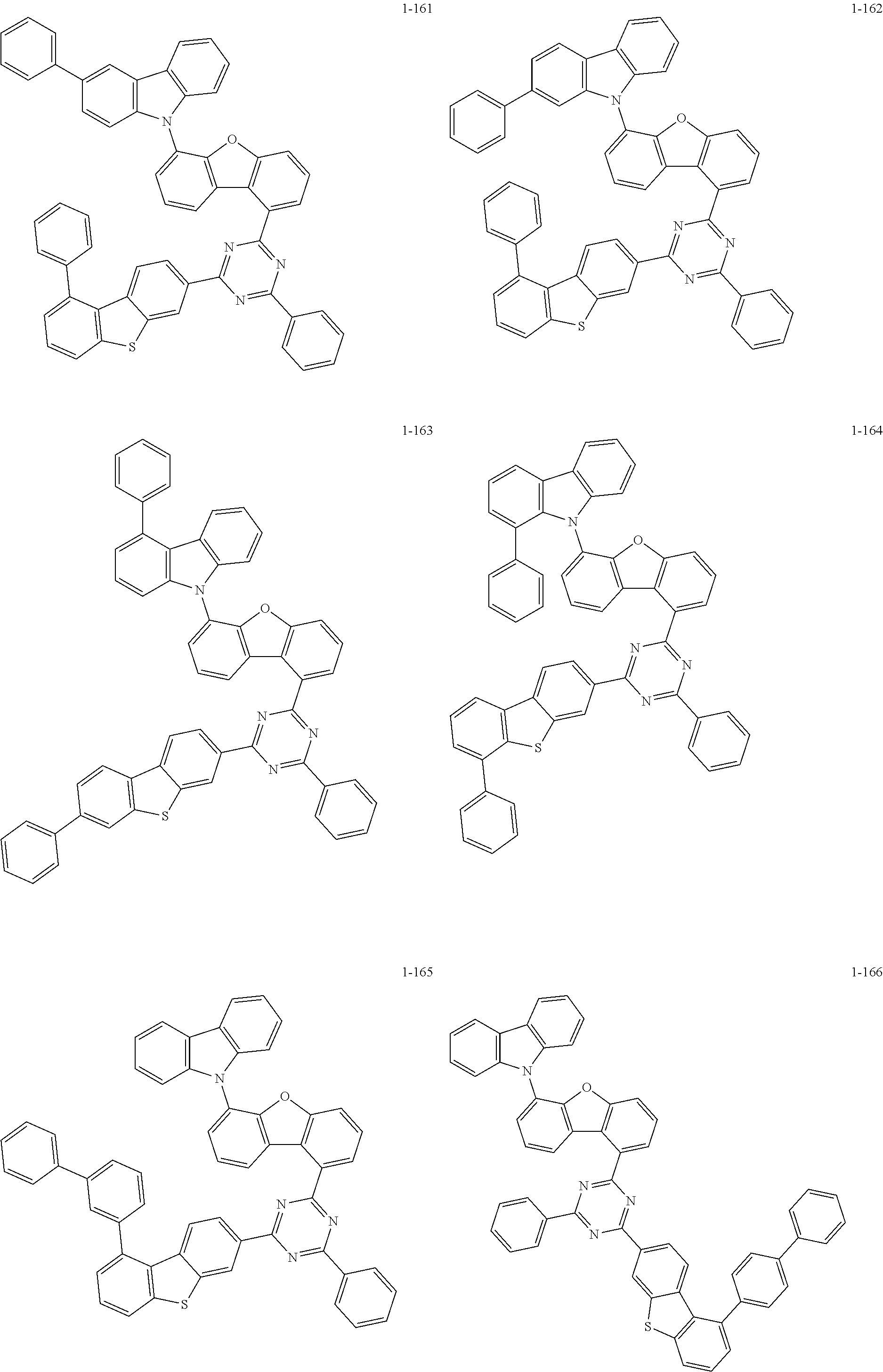

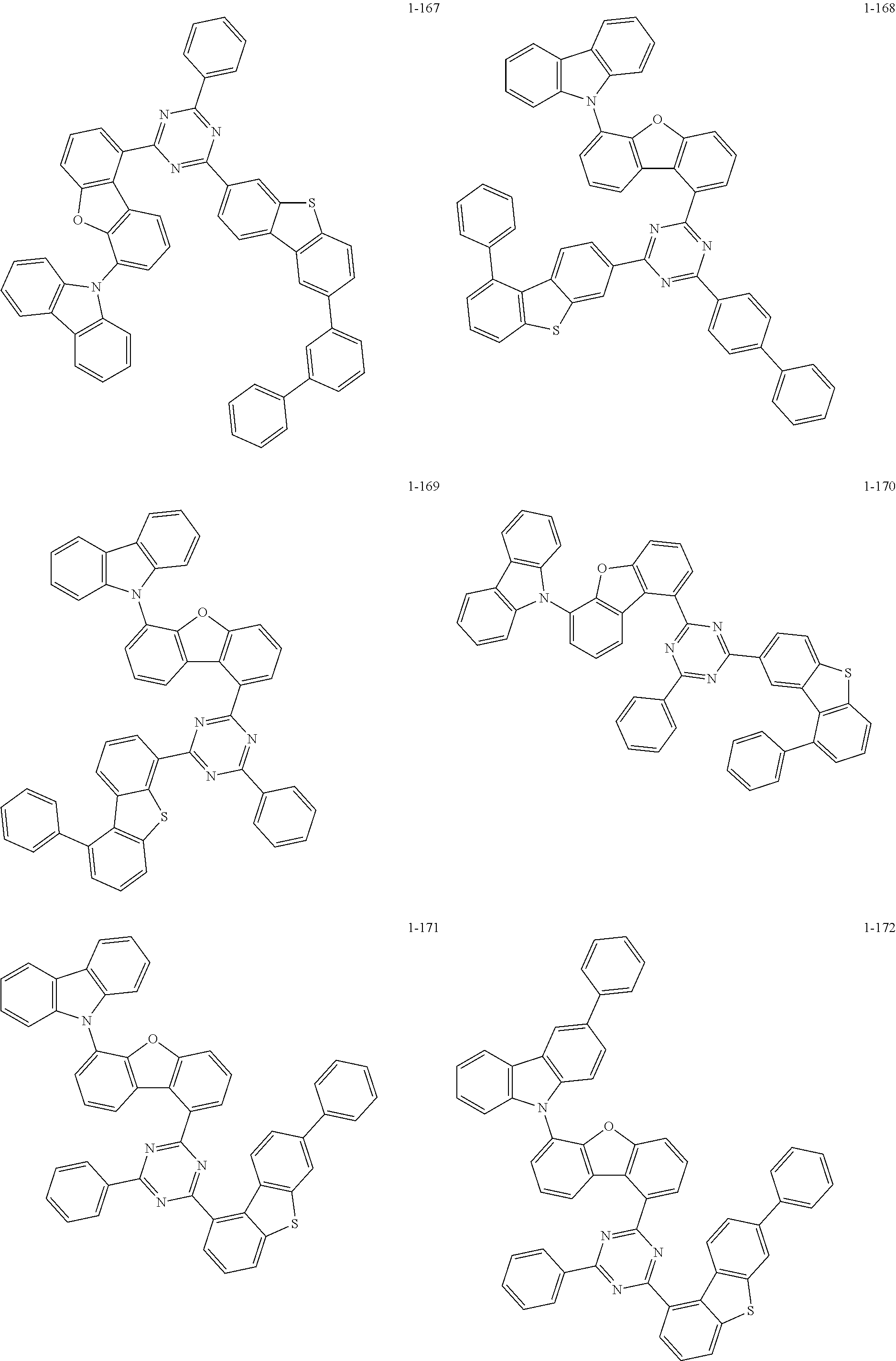

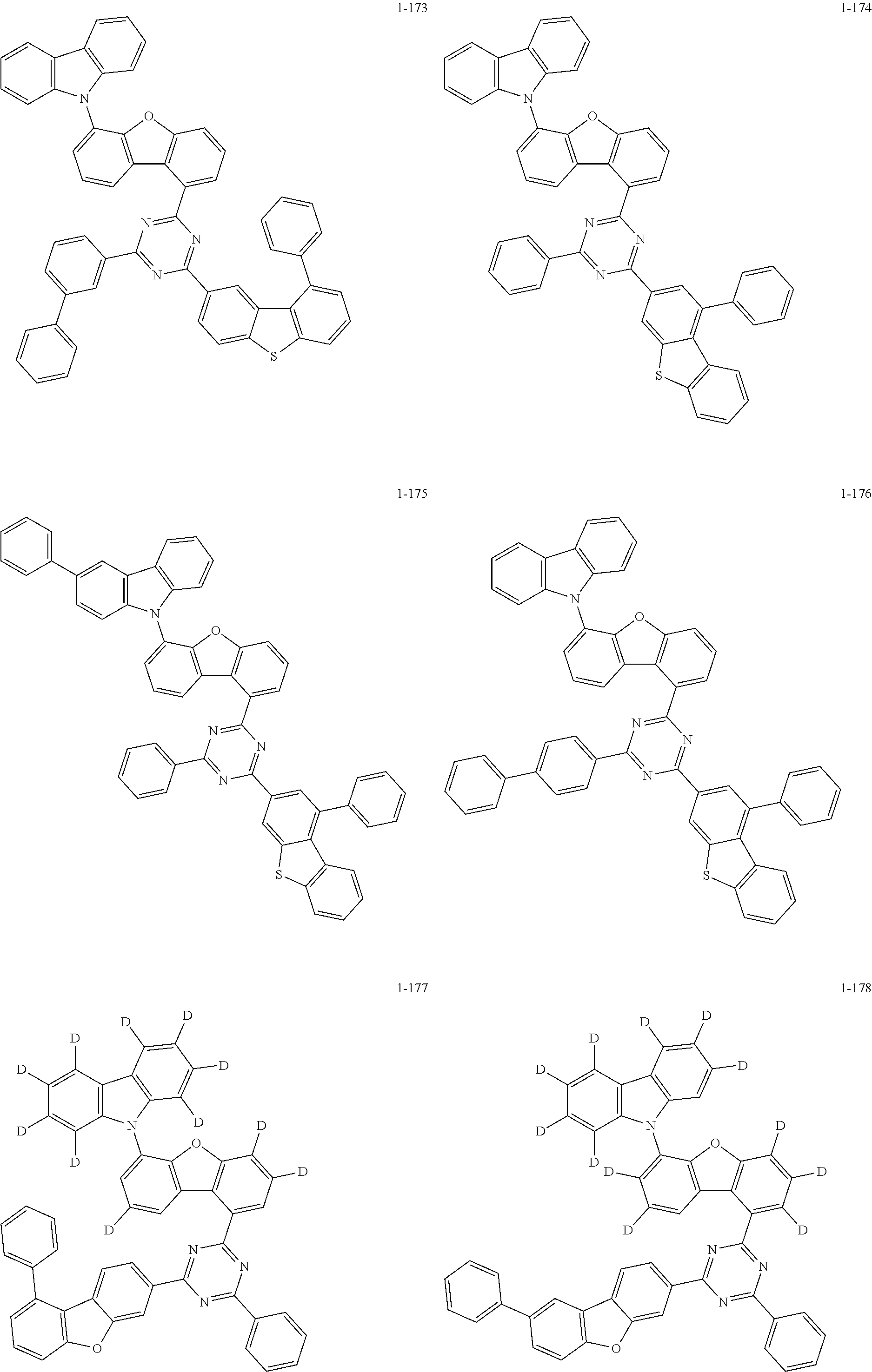

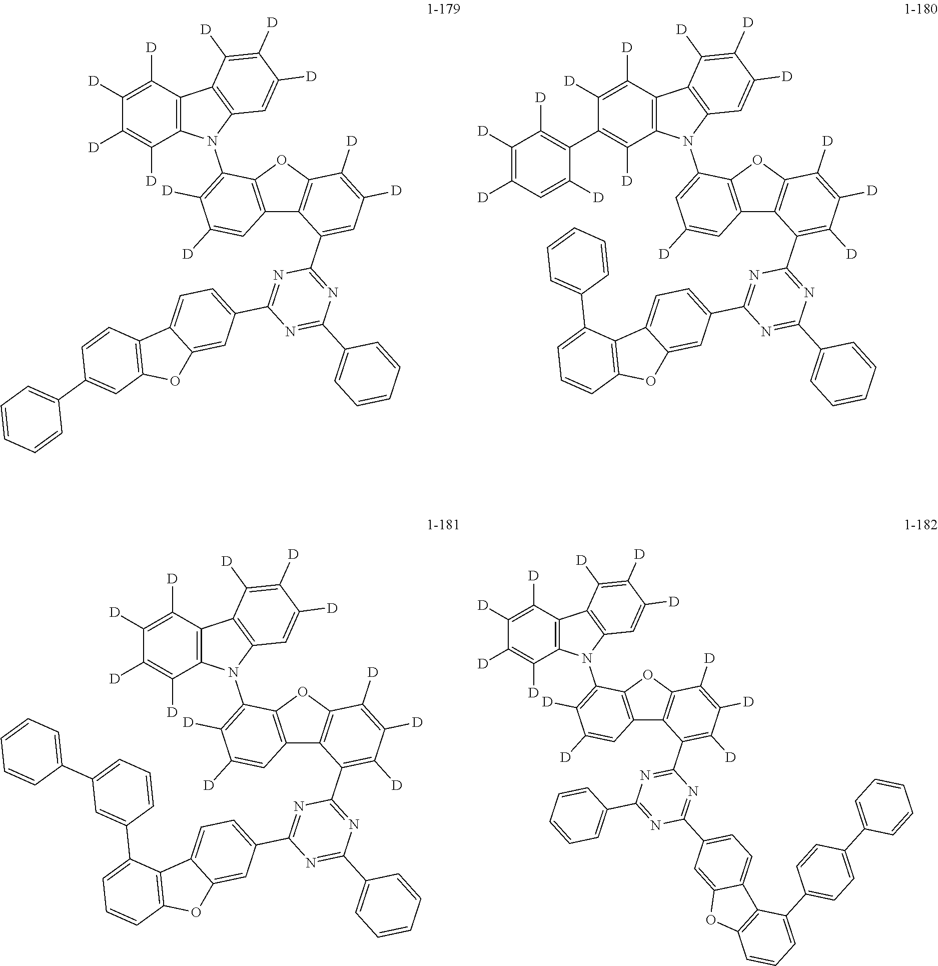

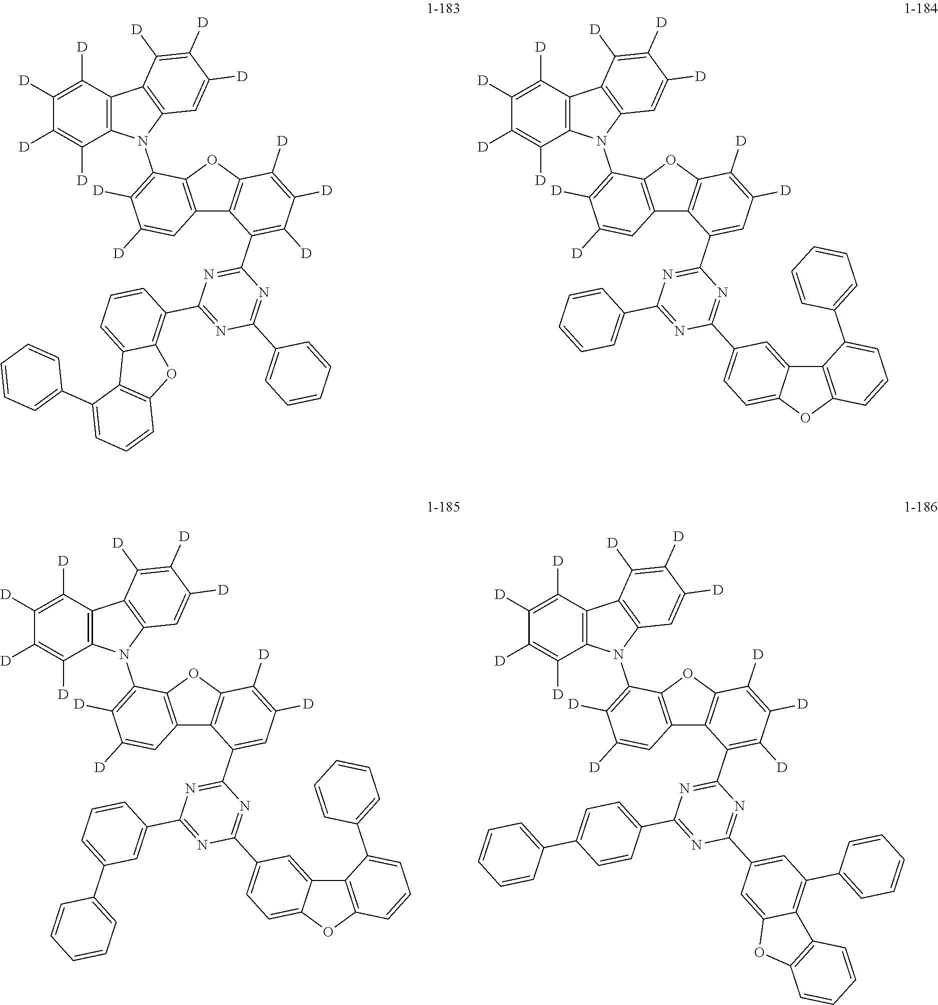

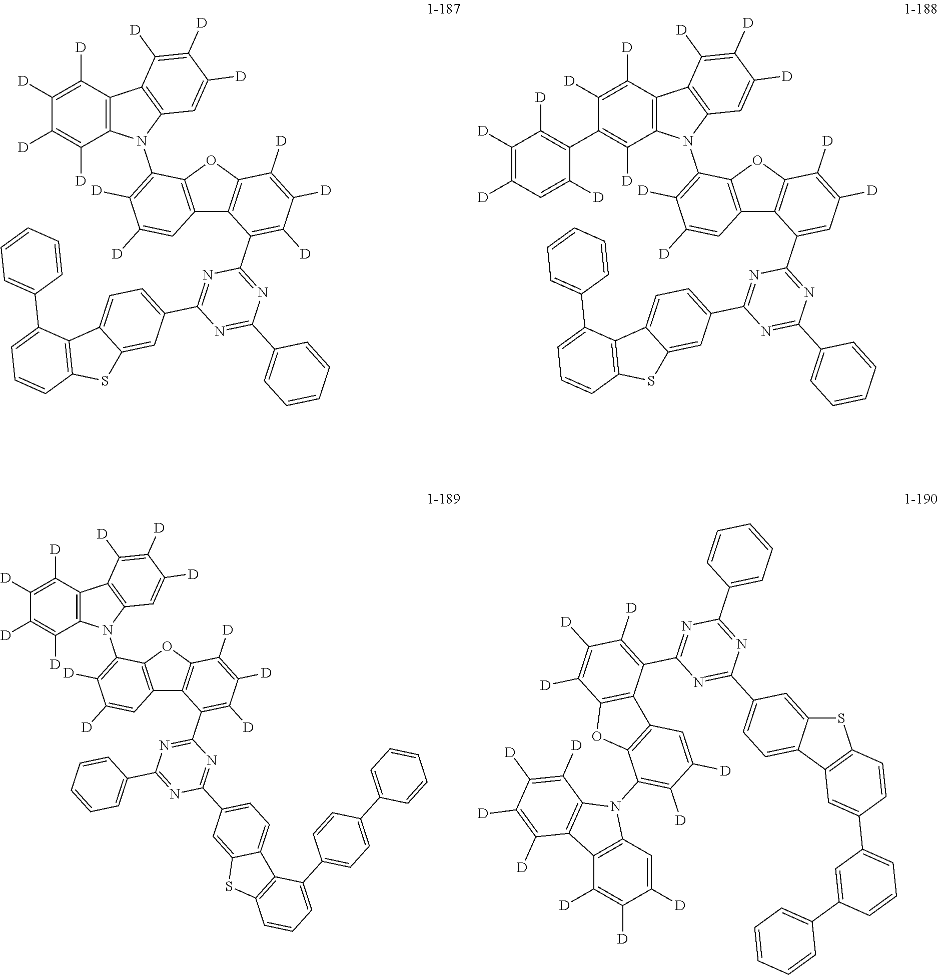

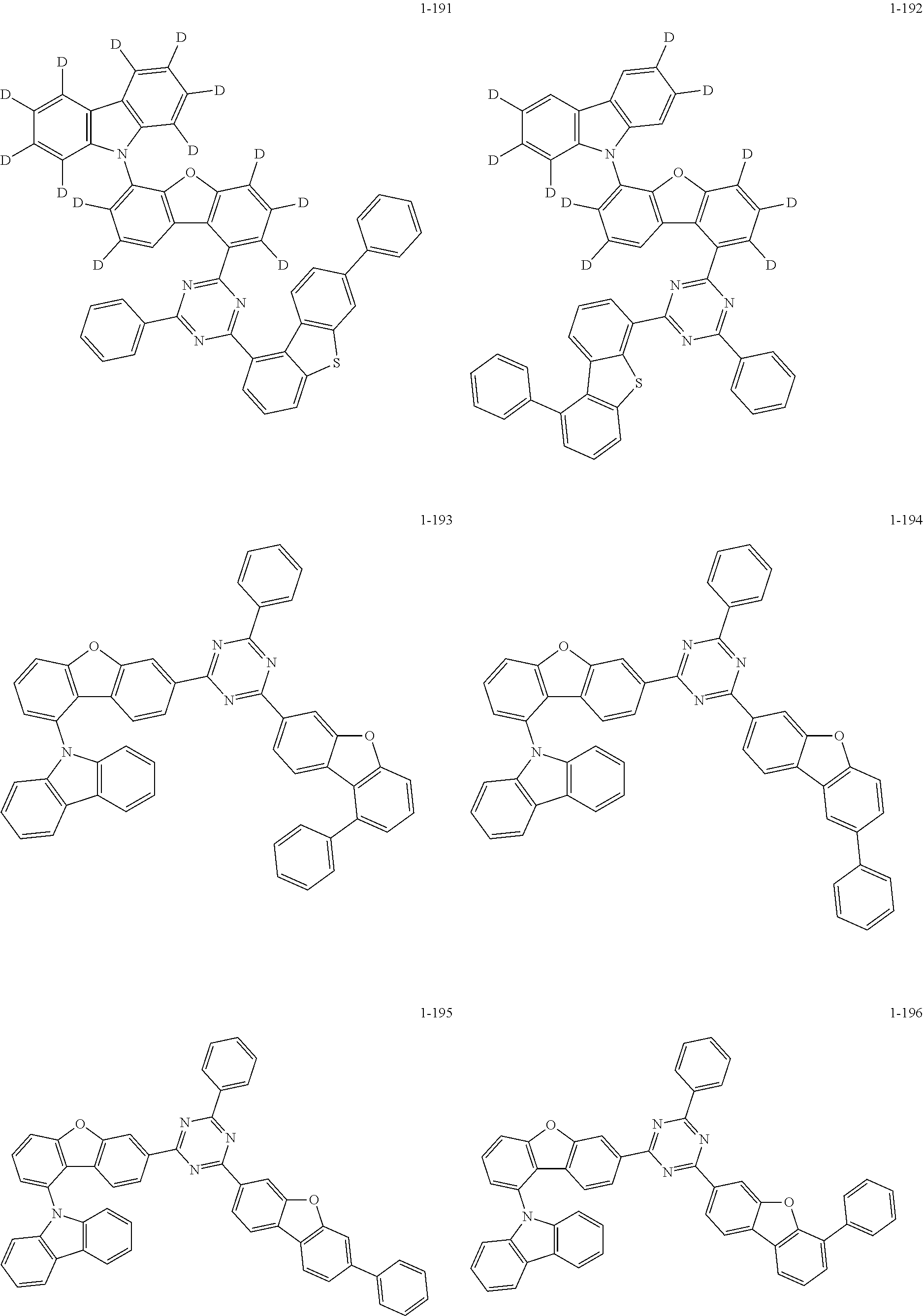

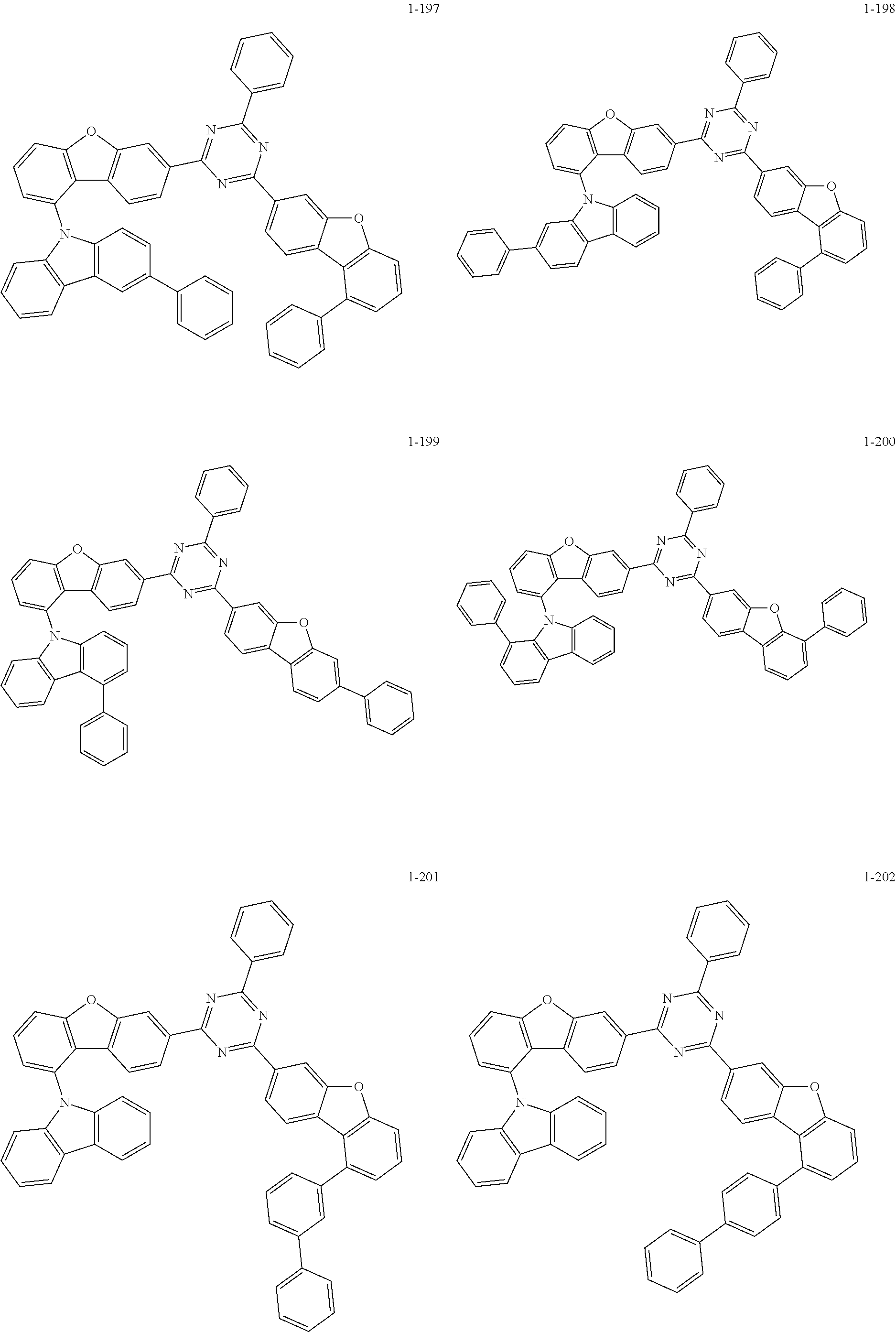

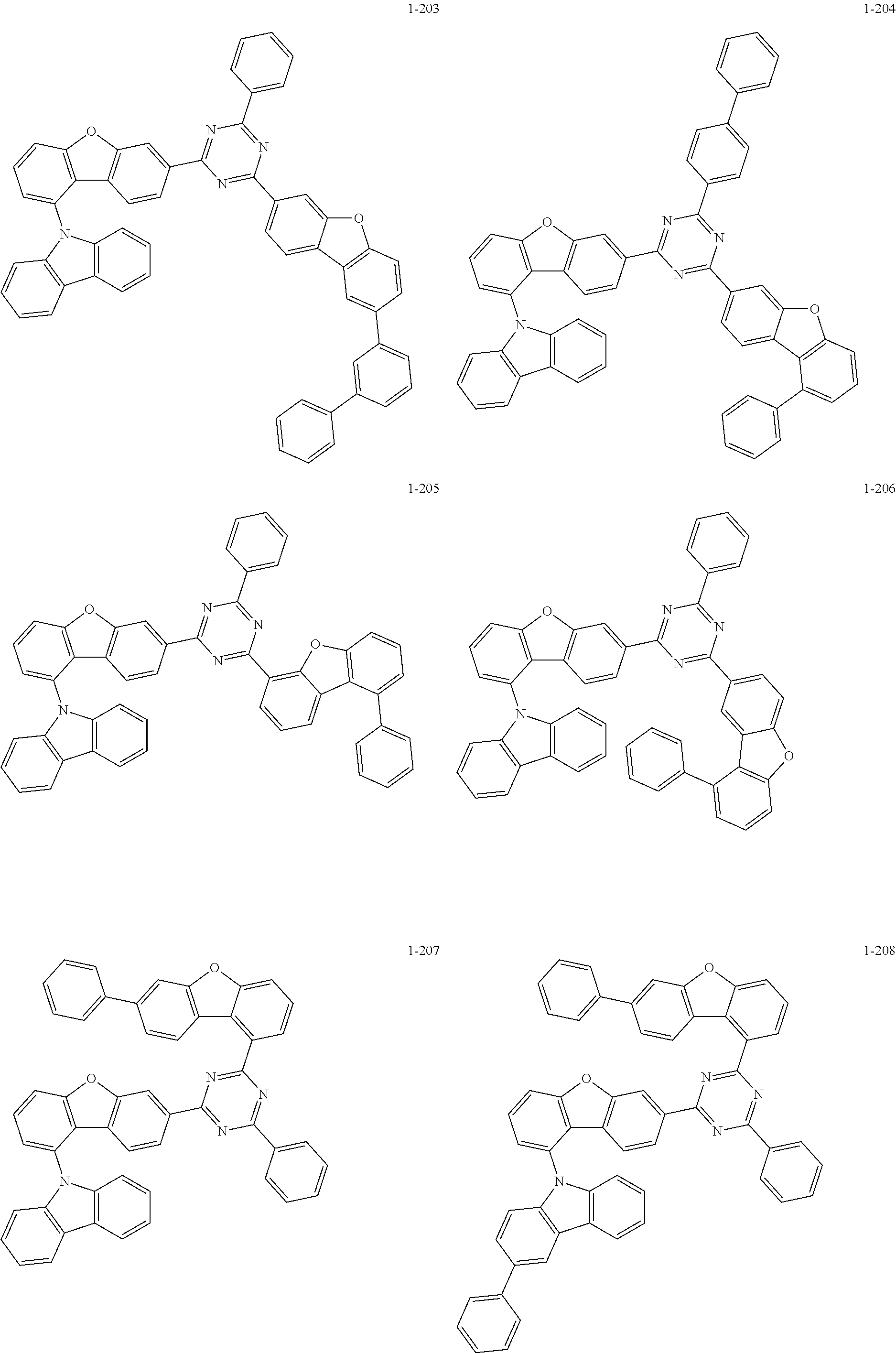

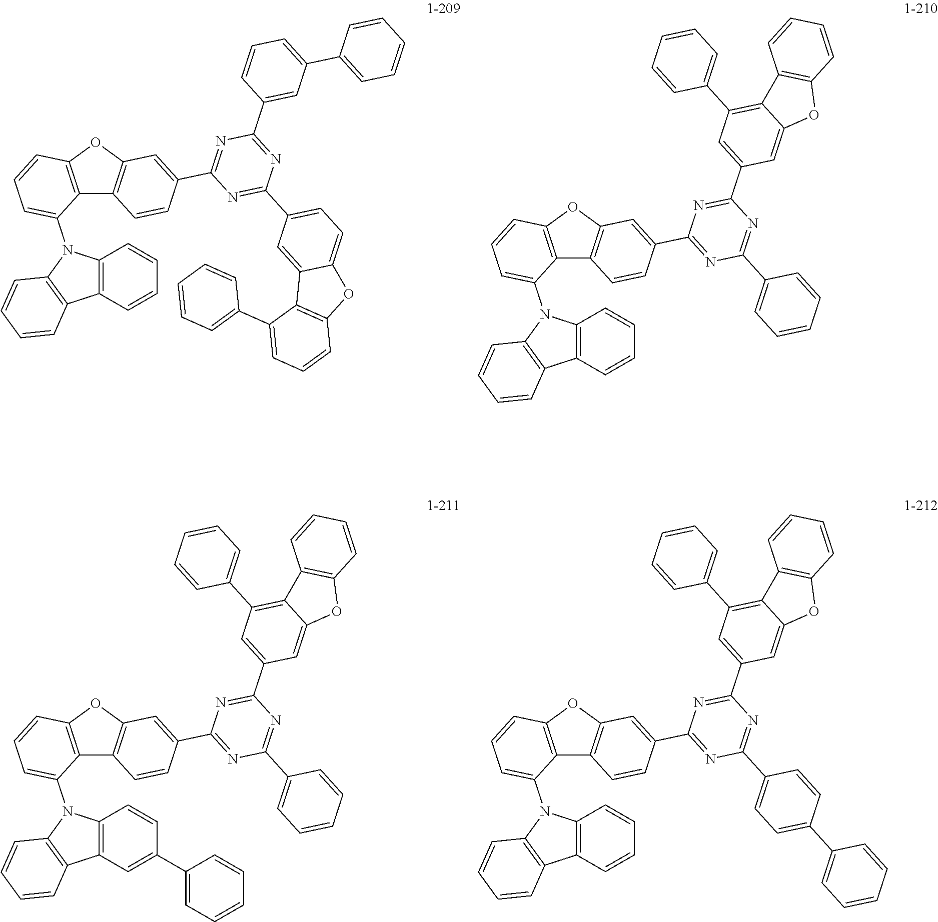 [SMILES: c1ccc(-c2cccc(-c3nc(-c4ccc5c(c4)oc4cccc(-n6c7ccccc7c7ccccc76)c45)nc(-c4ccc5oc6cccc(-c7ccccc7)c6c5c4)n3)c2)cc1 ; c1ccc(-c2nc(-c3ccc4c(c3)oc3cccc(-n5c6ccccc6c6ccccc65)c34)nc(-c3cc(-c4ccccc4)c4c(c3)oc3ccccc34)n2)cc1 ; c1ccc(-c2ccc3c(c2)c2ccccc2n3-c2cccc3oc4cc(-c5nc(-c6ccccc6)nc(-c6cc(-c7ccccc7)c7c(c6)oc6ccccc67)n5)ccc4c23)cc1 ; c1ccc(-c2ccc(-c3nc(-c4ccc5c(c4)oc4cccc(-n6c7ccccc7c7ccccc76)c45)nc(-c4cc(-c5ccccc5)c5c(c4)oc4ccccc45)n3)cc2)cc1]

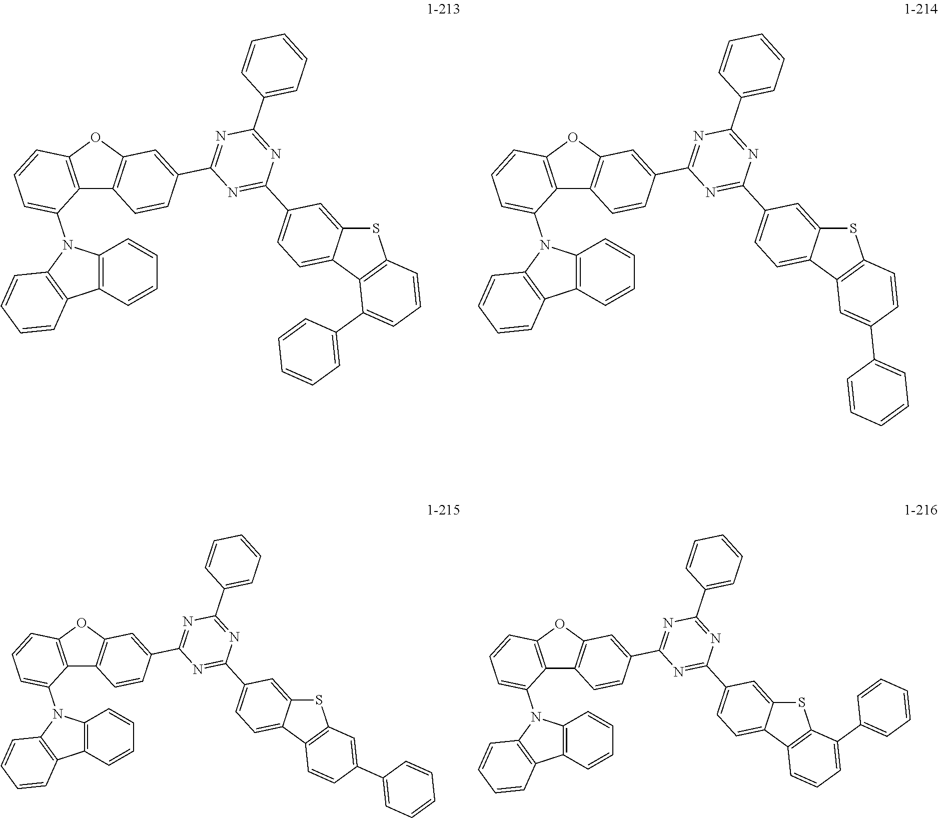 [SMILES: c1ccc(-c2nc(-c3ccc4c(c3)oc3cccc(-n5c6ccccc6c6ccccc65)c34)nc(-c3ccc4c(c3)sc3cccc(-c5ccccc5)c34)n2)cc1 ; c1ccc(-c2ccc3sc4cc(-c5nc(-c6ccccc6)nc(-c6ccc7c(c6)oc6cccc(-n8c9ccccc9c9ccccc98)c67)n5)ccc4c3c2)cc1 ; c1ccc(-c2ccc3c(c2)sc2cc(-c4nc(-c5ccccc5)nc(-c5ccc6c(c5)oc5cccc(-n7c8ccccc8c8ccccc87)c56)n4)ccc23)cc1 ; c1ccc(-c2nc(-c3ccc4c(c3)oc3cccc(-n5c6ccccc6c6ccccc65)c34)nc(-c3ccc4c(c3)sc3c(-c5ccccc5)cccc34)n2)cc1]

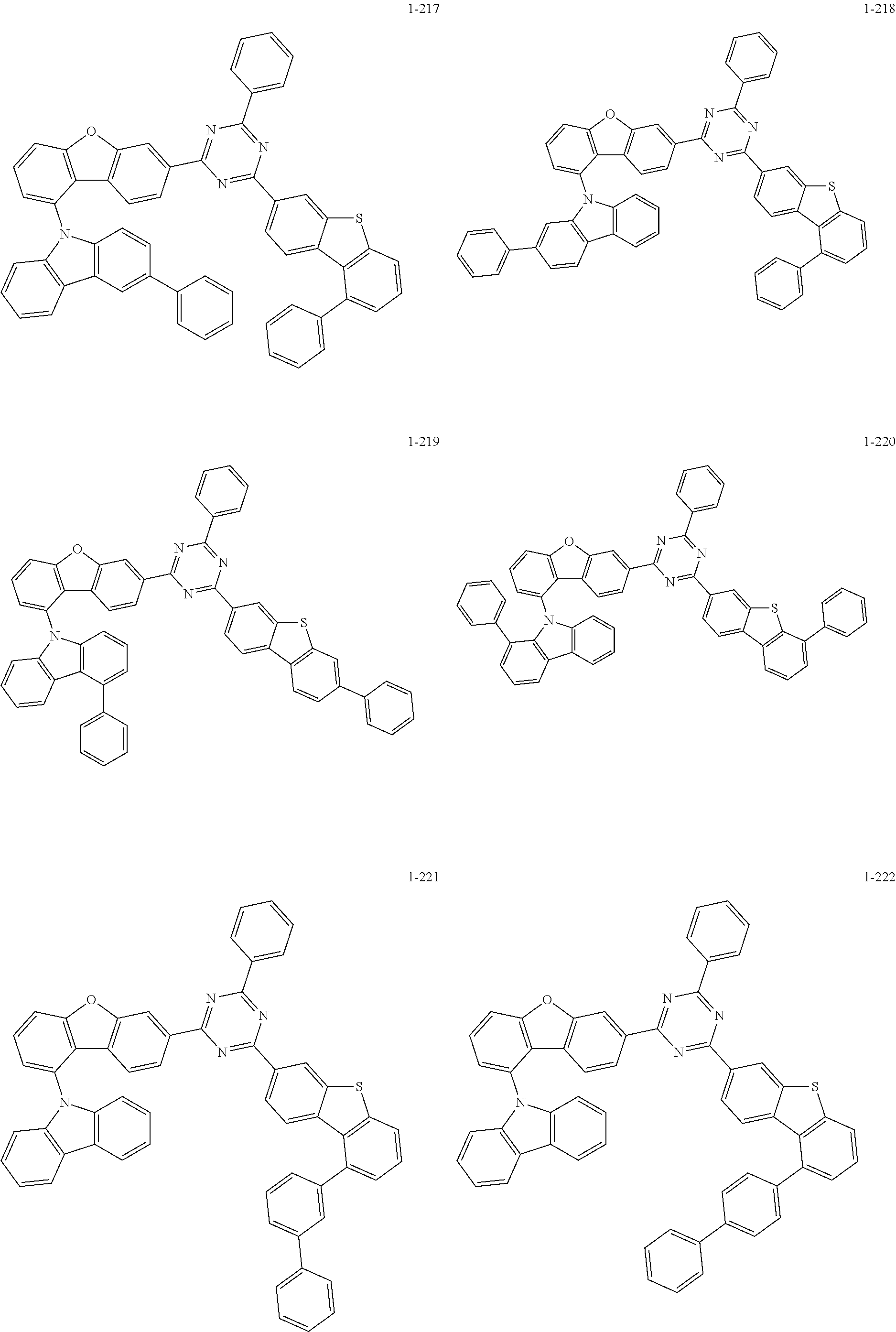

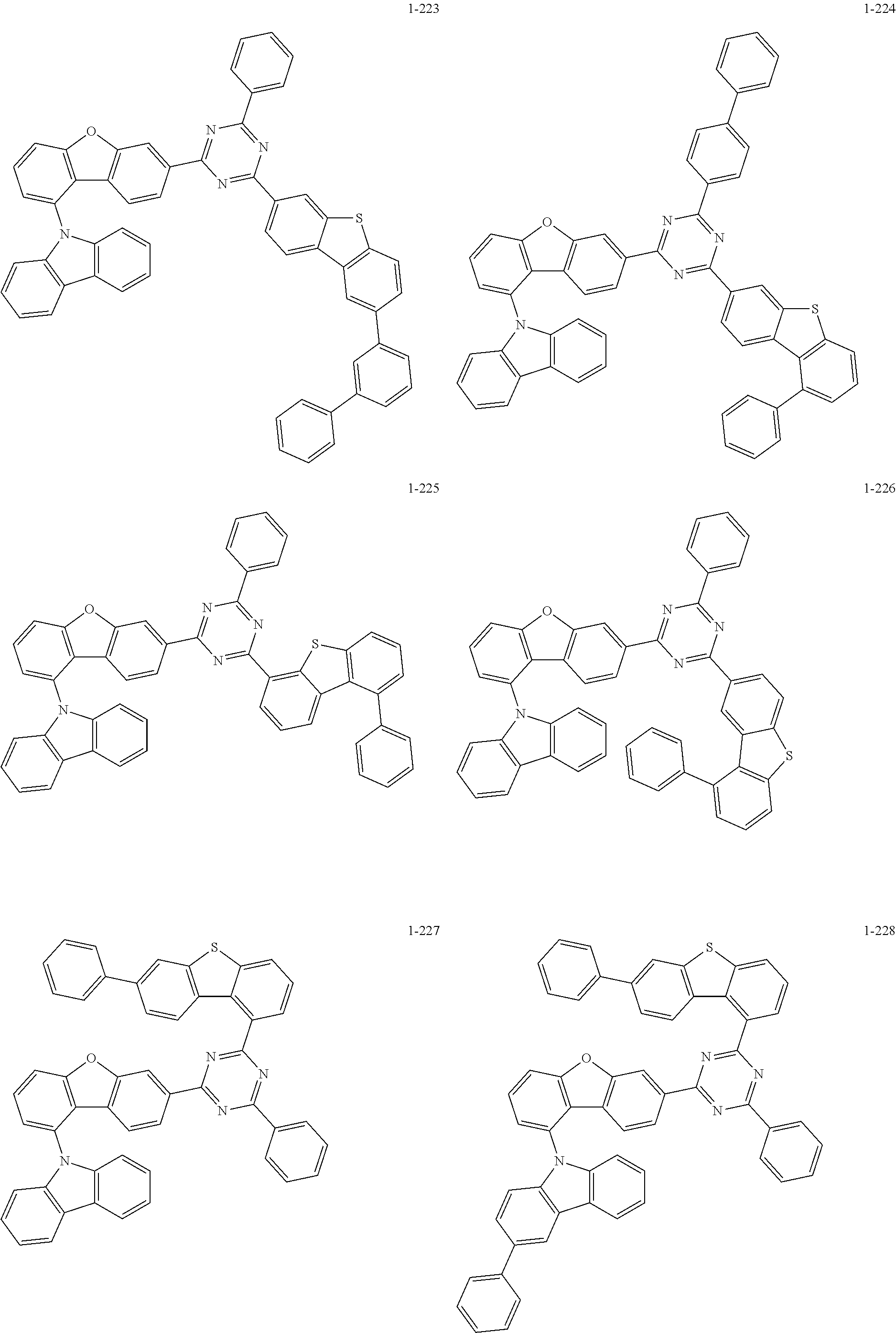

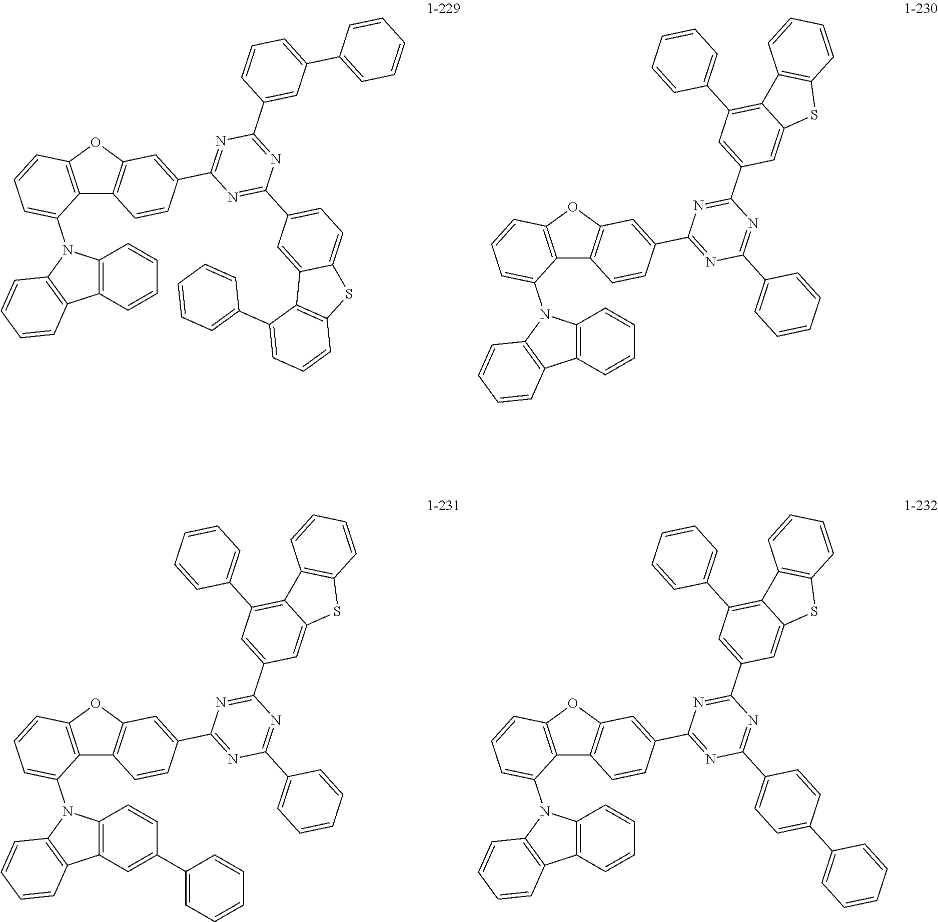 [SMILES: c1ccc(-c2cccc(-c3nc(-c4ccc5c(c4)oc4cccc(-n6c7ccccc7c7ccccc76)c45)nc(-c4ccc5sc6cccc(-c7ccccc7)c6c5c4)n3)c2)cc1 ; c1ccc(-c2nc(-c3ccc4c(c3)oc3cccc(-n5c6ccccc6c6ccccc65)c34)nc(-c3cc(-c4ccccc4)c4c(c3)sc3ccccc34)n2)cc1 ; c1ccc(-c2ccc3c(c2)c2ccccc2n3-c2cccc3oc4cc(-c5nc(-c6ccccc6)nc(-c6cc(-c7ccccc7)c7c(c6)sc6ccccc67)n5)ccc4c23)cc1 ; c1ccc(-c2ccc(-c3nc(-c4ccc5c(c4)oc4cccc(-n6c7ccccc7c7ccccc76)c45)nc(-c4cc(-c5ccccc5)c5c(c4)sc4ccccc45)n3)cc2)cc1]

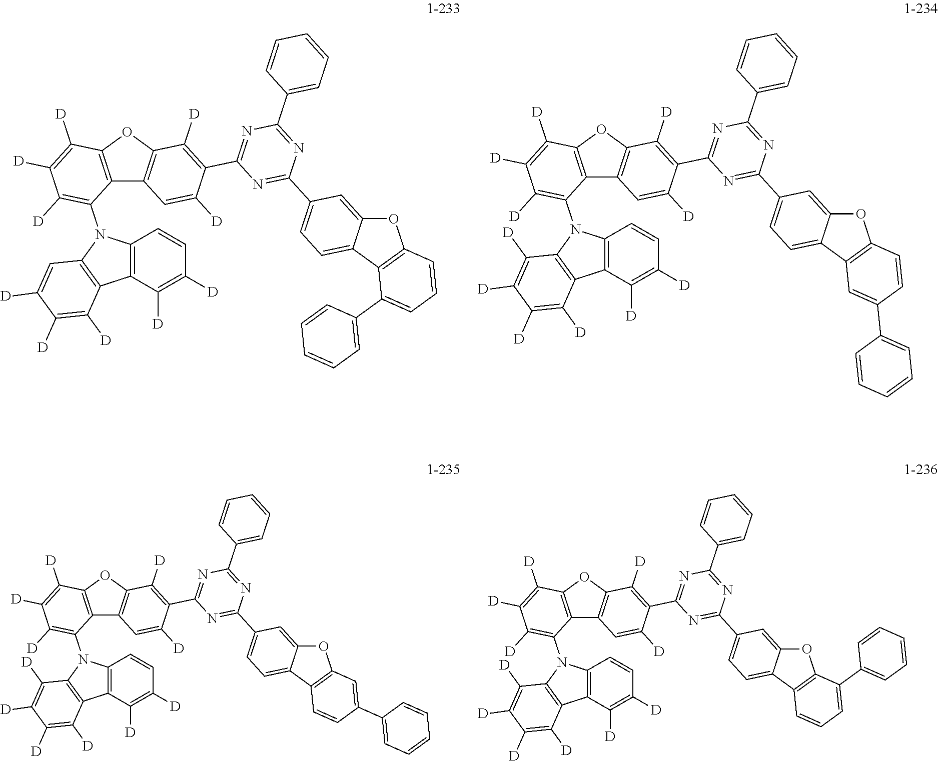

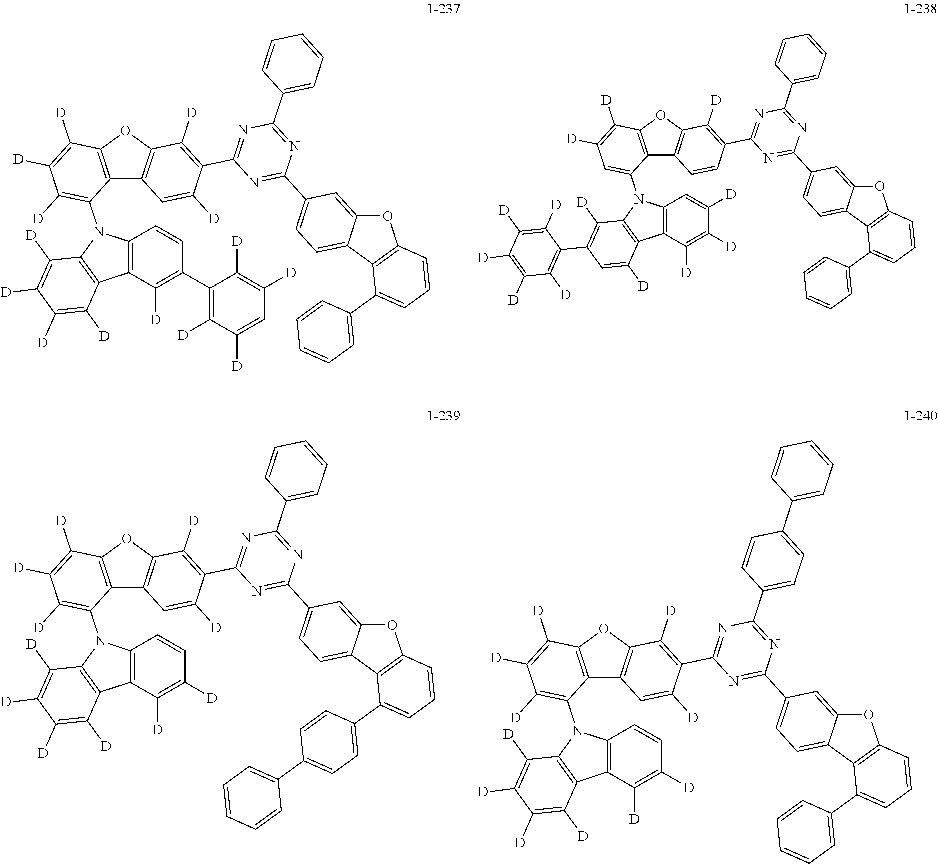

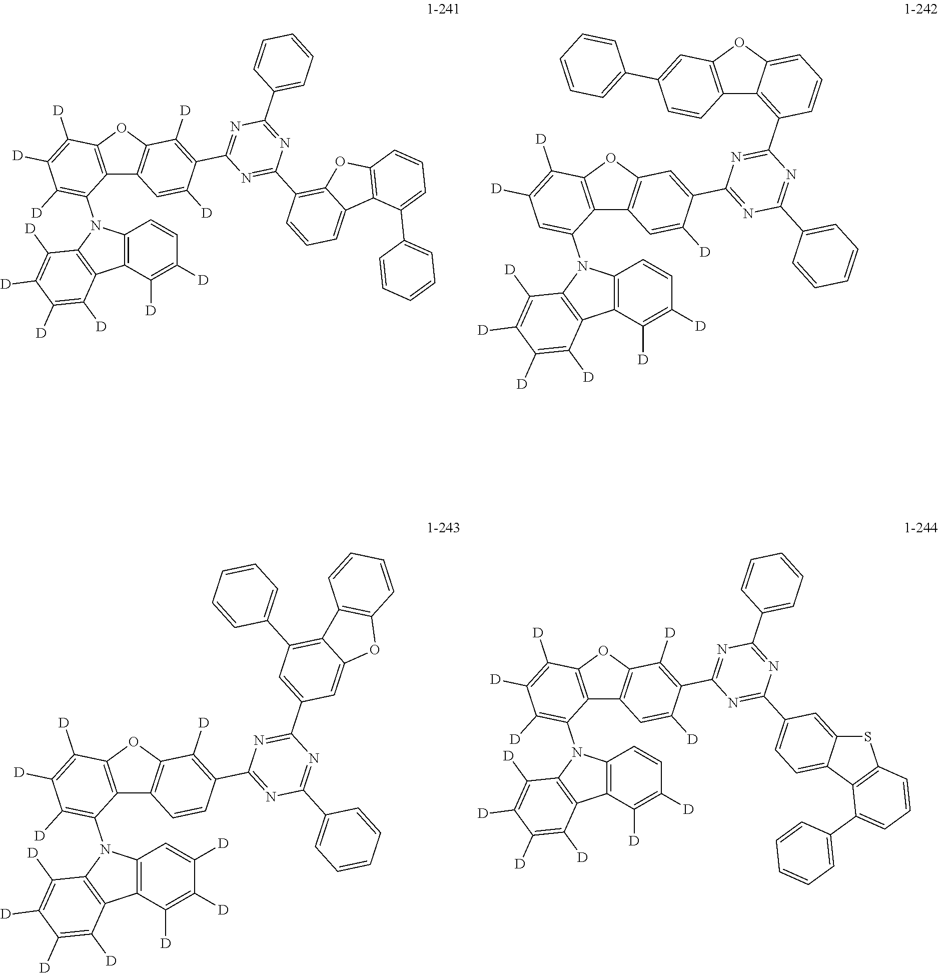

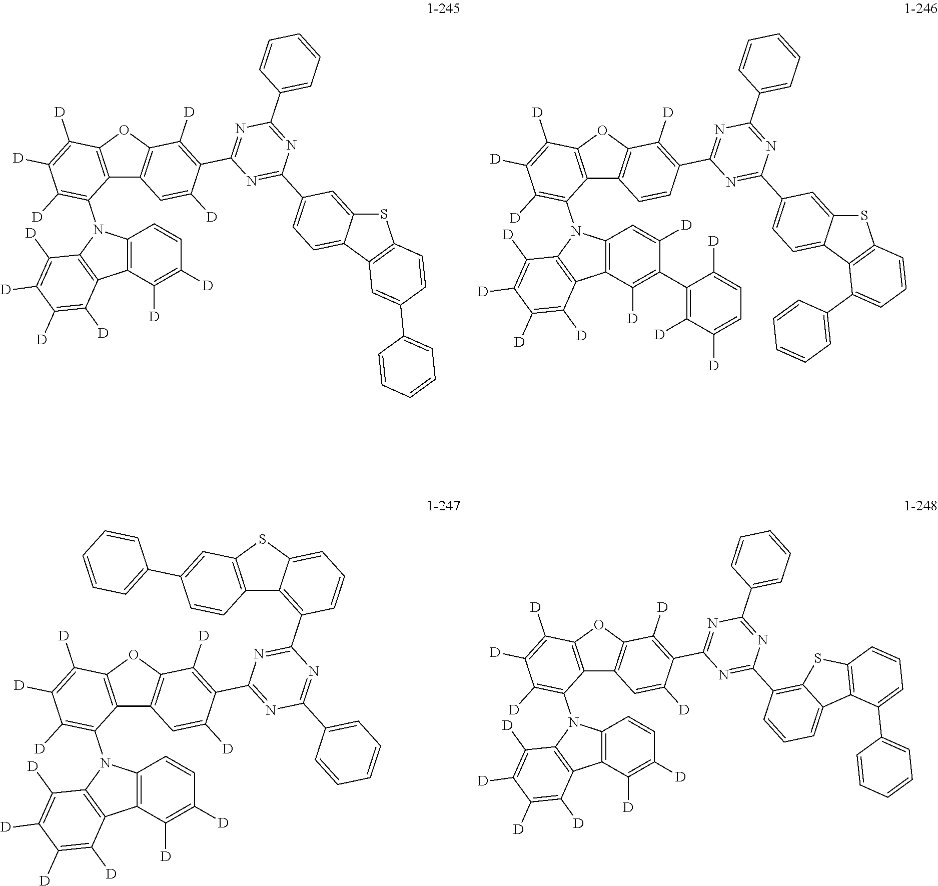

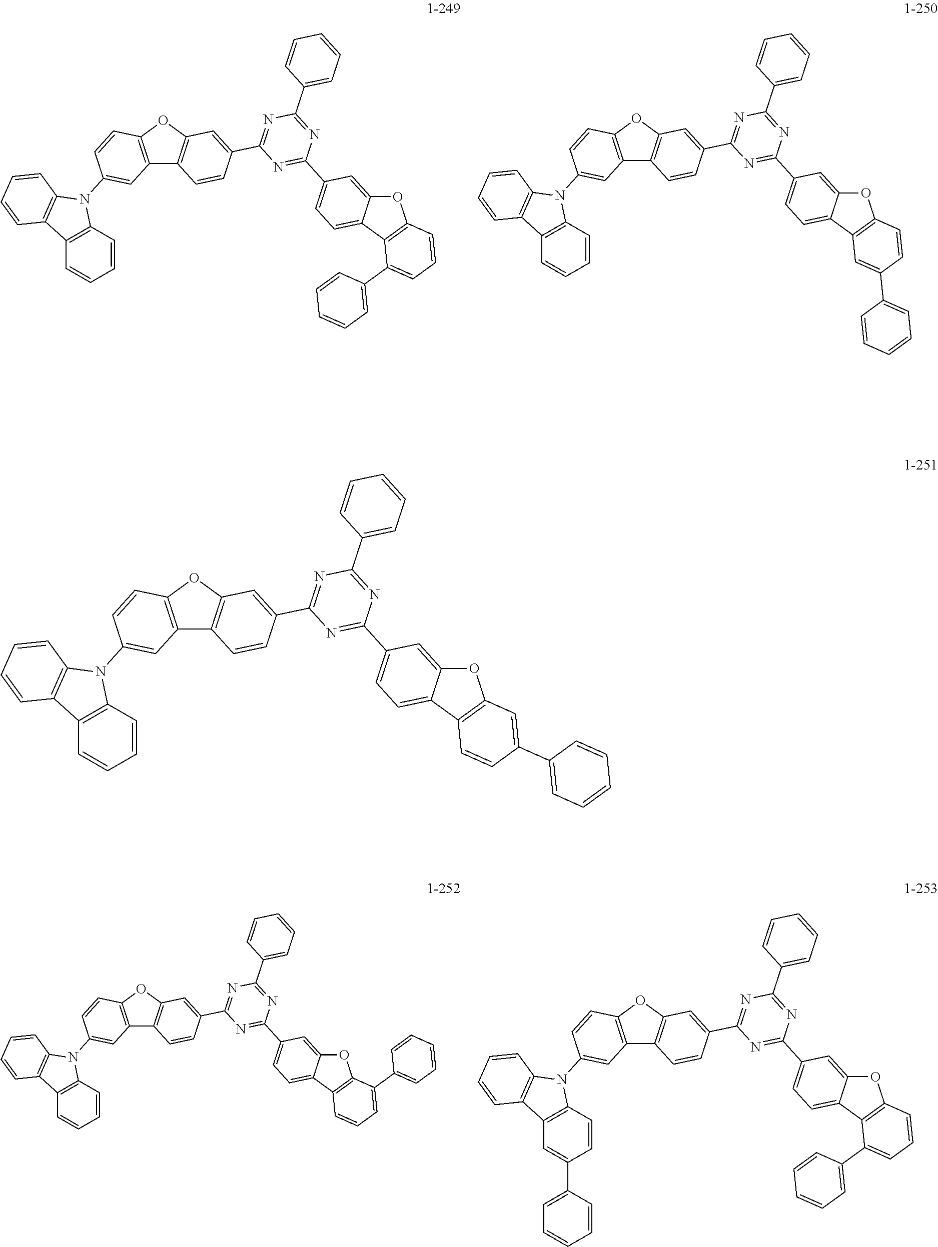

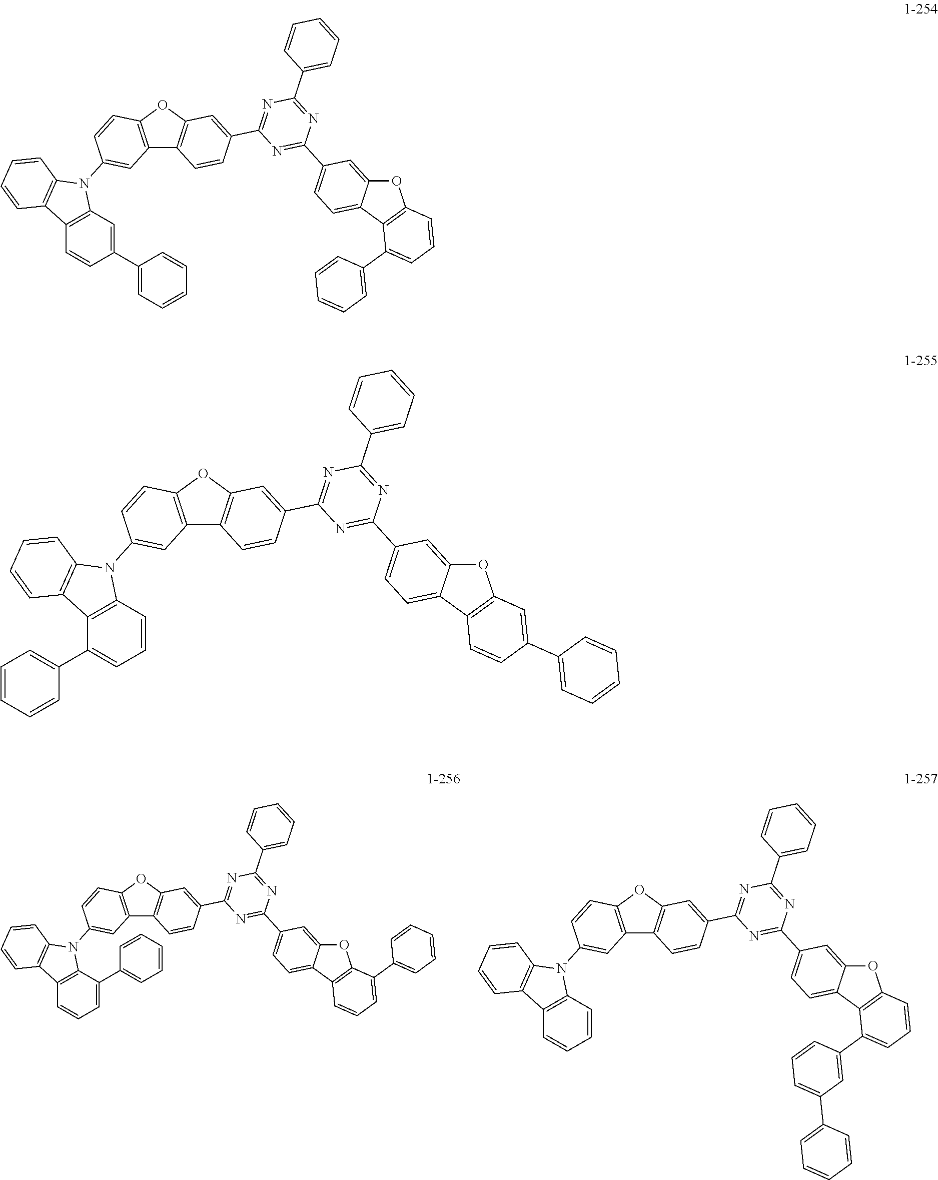 [SMILES: c1ccc(-c2ccc3c4ccccc4n(-c4ccc5oc6cc(-c7nc(-c8ccccc8)nc(-c8ccc9c(c8)oc8cccc(-c%10ccccc%10)c89)n7)ccc6c5c4)c3c2)cc1 ; c1ccc(-c2ccc3c(c2)oc2cc(-c4nc(-c5ccccc5)nc(-c5ccc6c(c5)oc5ccc(-n7c8ccccc8c8c(-c9ccccc9)cccc87)cc56)n4)ccc23)cc1 ; c1ccc(-c2nc(-c3ccc4c(c3)oc3ccc(-n5c6ccccc6c6cccc(-c7ccccc7)c65)cc34)nc(-c3ccc4c(c3)oc3c(-c5ccccc5)cccc34)n2)cc1 ; c1ccc(-c2cccc(-c3cccc4oc5cc(-c6nc(-c7ccccc7)nc(-c7ccc8c(c7)oc7ccc(-n9c%10ccccc%10c%10ccccc%109)cc78)n6)ccc5c34)c2)cc1]

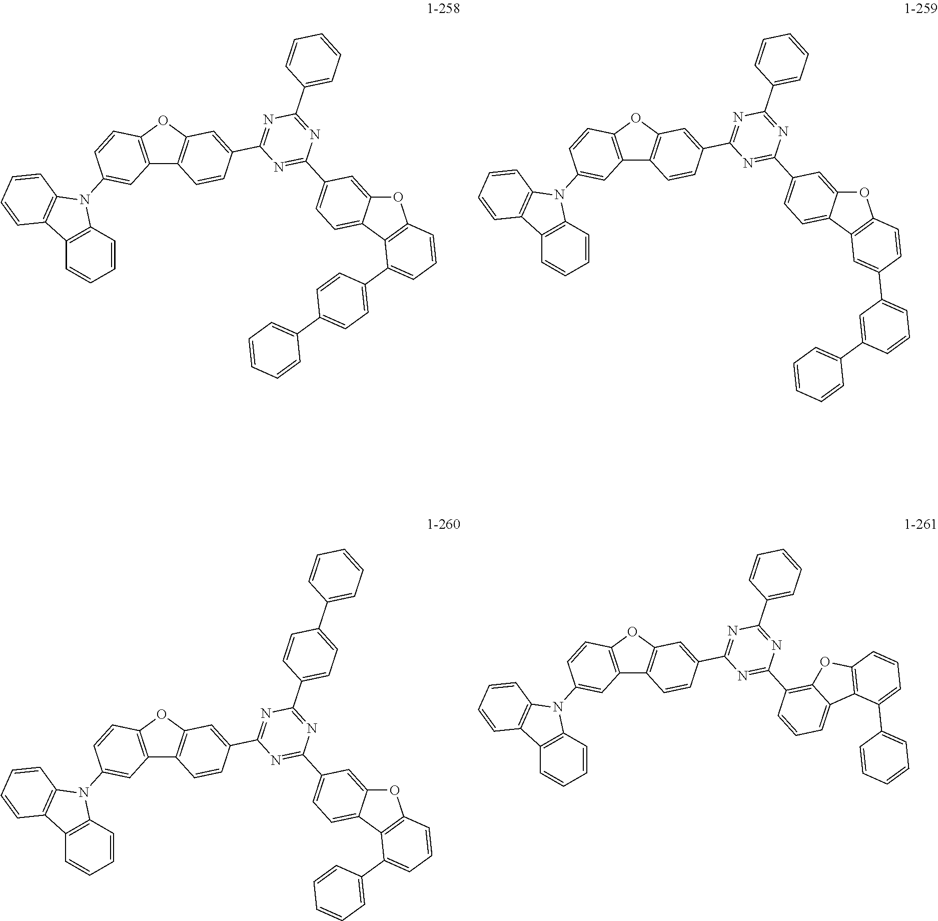 [SMILES: c1ccc(-c2ccc(-c3cccc4oc5cc(-c6nc(-c7ccccc7)nc(-c7ccc8c(c7)oc7ccc(-n9c%10ccccc%10c%10ccccc%109)cc78)n6)ccc5c34)cc2)cc1 ; c1ccc(-c2cccc(-c3ccc4oc5cc(-c6nc(-c7ccccc7)nc(-c7ccc8c(c7)oc7ccc(-n9c%10ccccc%10c%10ccccc%109)cc78)n6)ccc5c4c3)c2)cc1 ; c1ccc(-c2ccc(-c3nc(-c4ccc5c(c4)oc4ccc(-n6c7ccccc7c7ccccc76)cc45)nc(-c4ccc5c(c4)oc4cccc(-c6ccccc6)c45)n3)cc2)cc1 ; c1ccc(-c2nc(-c3ccc4c(c3)oc3ccc(-n5c6ccccc6c6ccccc65)cc34)nc(-c3cccc4c3oc3cccc(-c5ccccc5)c34)n2)cc1]

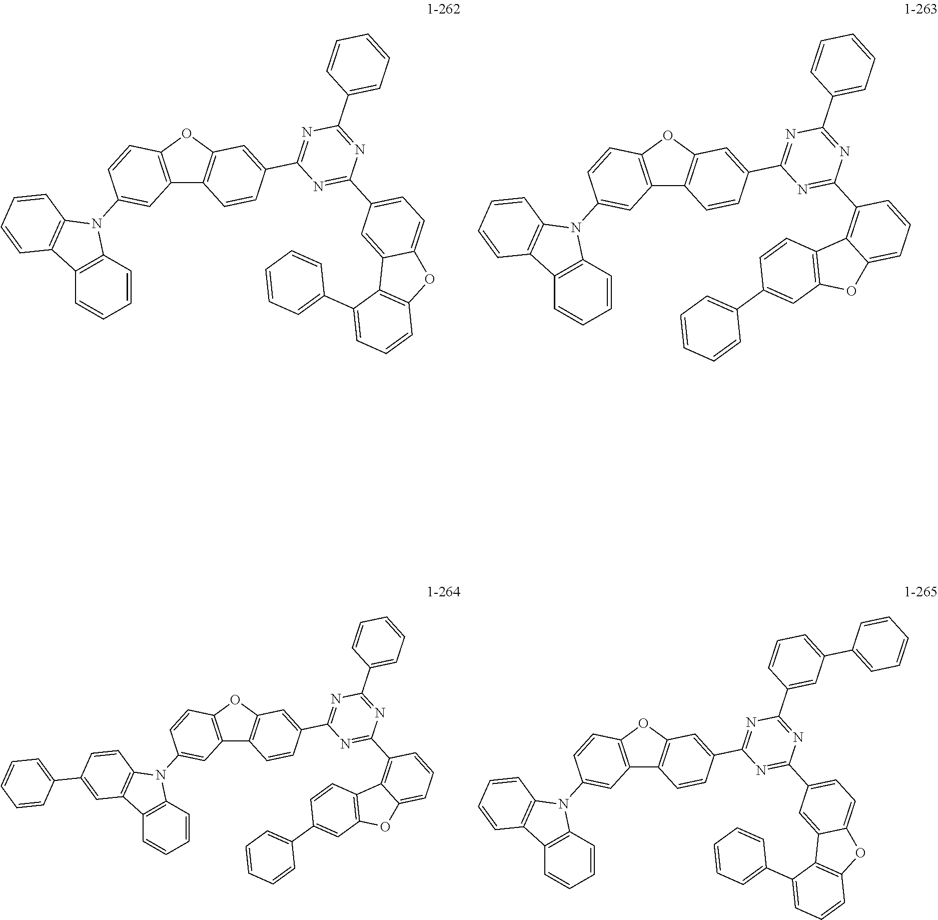 [SMILES: c1ccc(-c2nc(-c3ccc4c(c3)oc3ccc(-n5c6ccccc6c6ccccc65)cc34)nc(-c3ccc4oc5cccc(-c6ccccc6)c5c4c3)n2)cc1 ; c1ccc(-c2ccc3c(c2)oc2cccc(-c4nc(-c5ccccc5)nc(-c5ccc6c(c5)oc5ccc(-n7c8ccccc8c8ccccc87)cc56)n4)c23)cc1 ; c1ccc(-c2ccc3c(c2)oc2cccc(-c4nc(-c5ccccc5)nc(-c5ccc6c(c5)oc5ccc(-n7c8ccccc8c8cc(-c9ccccc9)ccc87)cc56)n4)c23)cc1 ; c1ccc(-c2cccc(-c3nc(-c4ccc5c(c4)oc4ccc(-n6c7ccccc7c7ccccc76)cc45)nc(-c4ccc5oc6cccc(-c7ccccc7)c6c5c4)n3)c2)cc1]

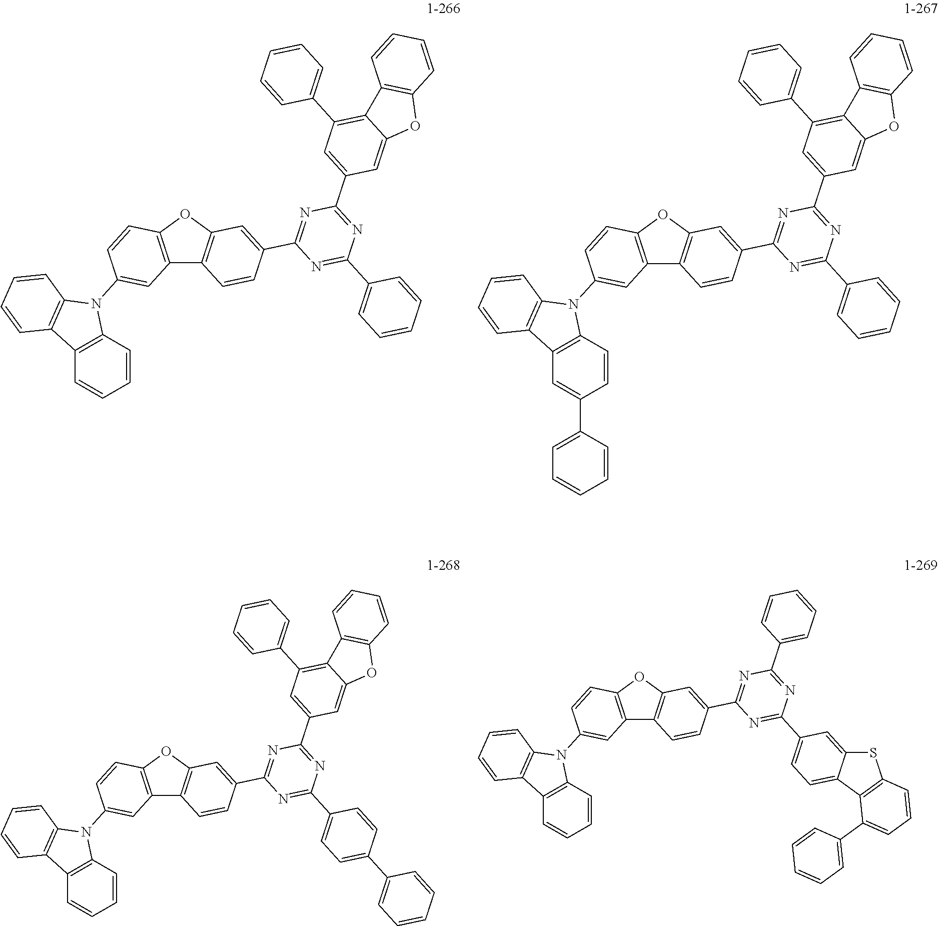 [SMILES: c1ccc(-c2nc(-c3ccc4c(c3)oc3ccc(-n5c6ccccc6c6ccccc65)cc34)nc(-c3cc(-c4ccccc4)c4c(c3)oc3ccccc34)n2)cc1 ; c1ccc(-c2ccc3c(c2)c2ccccc2n3-c2ccc3oc4cc(-c5nc(-c6ccccc6)nc(-c6cc(-c7ccccc7)c7c(c6)oc6ccccc67)n5)ccc4c3c2)cc1 ; c1ccc(-c2ccc(-c3nc(-c4ccc5c(c4)oc4ccc(-n6c7ccccc7c7ccccc76)cc45)nc(-c4cc(-c5ccccc5)c5c(c4)oc4ccccc45)n3)cc2)cc1 ; c1ccc(-c2nc(-c3ccc4c(c3)oc3ccc(-n5c6ccccc6c6ccccc65)cc34)nc(-c3ccc4c(c3)sc3cccc(-c5ccccc5)c34)n2)cc1]

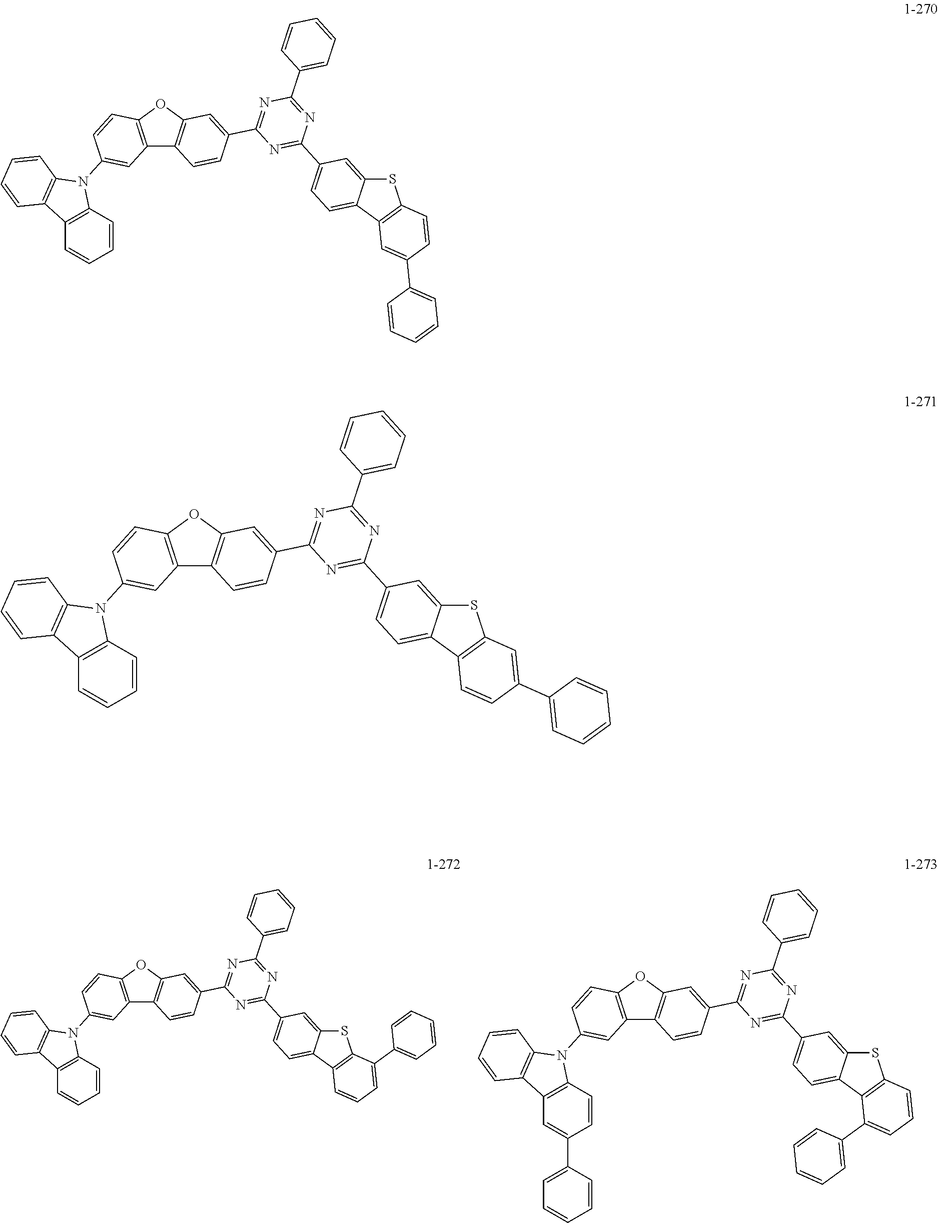 [SMILES: c1ccc(-c2ccc3sc4cc(-c5nc(-c6ccccc6)nc(-c6ccc7c(c6)oc6ccc(-n8c9ccccc9c9ccccc98)cc67)n5)ccc4c3c2)cc1 ; c1ccc(-c2ccc3c(c2)sc2cc(-c4nc(-c5ccccc5)nc(-c5ccc6c(c5)oc5ccc(-n7c8ccccc8c8ccccc87)cc56)n4)ccc23)cc1 ; c1ccc(-c2nc(-c3ccc4c(c3)oc3ccc(-n5c6ccccc6c6ccccc65)cc34)nc(-c3ccc4c(c3)sc3c(-c5ccccc5)cccc34)n2)cc1 ; c1ccc(-c2ccc3c(c2)c2ccccc2n3-c2ccc3oc4cc(-c5nc(-c6ccccc6)nc(-c6ccc7c(c6)sc6cccc(-c8ccccc8)c67)n5)ccc4c3c2)cc1]

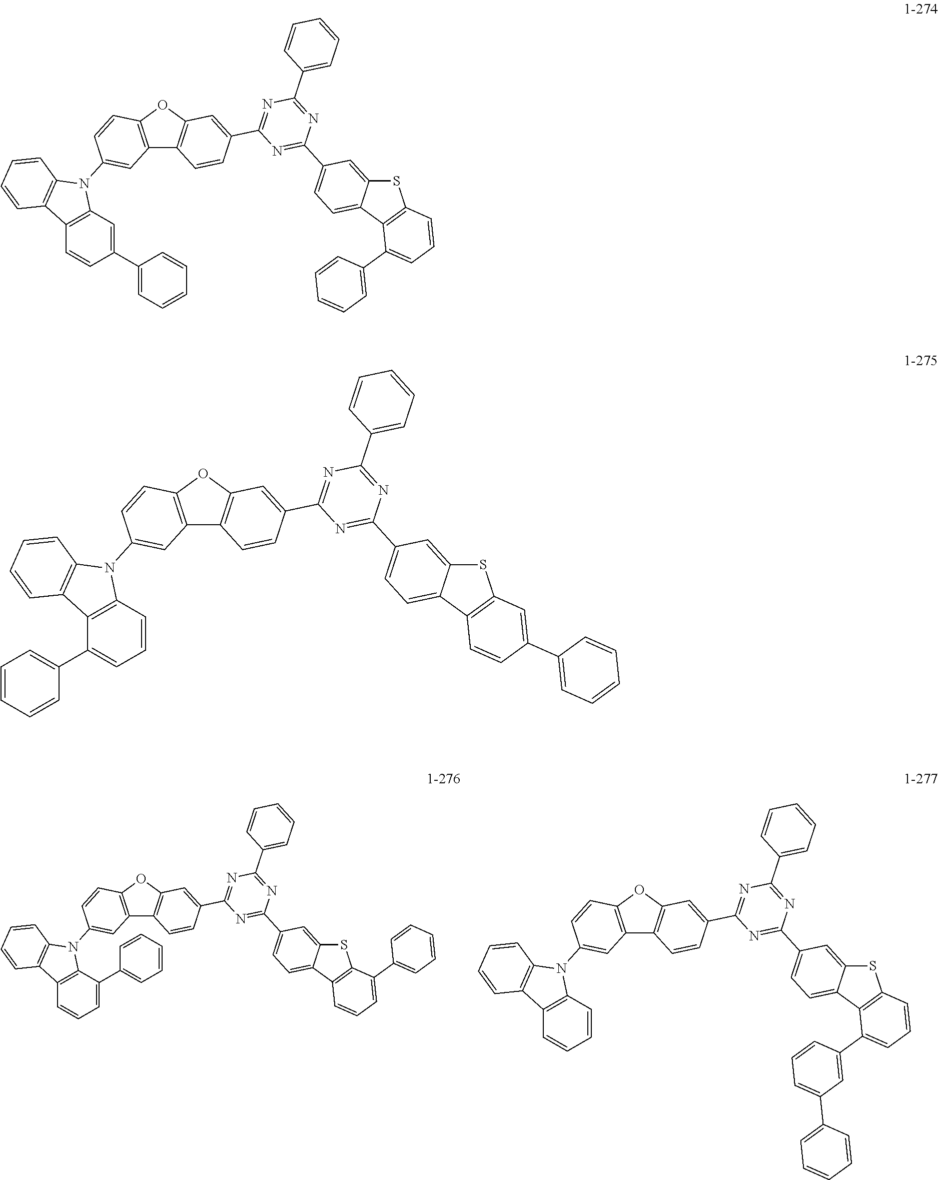 [SMILES: c1ccc(-c2ccc3c4ccccc4n(-c4ccc5oc6cc(-c7nc(-c8ccccc8)nc(-c8ccc9c(c8)sc8cccc(-c%10ccccc%10)c89)n7)ccc6c5c4)c3c2)cc1 ; c1ccc(-c2ccc3c(c2)sc2cc(-c4nc(-c5ccccc5)nc(-c5ccc6c(c5)oc5ccc(-n7c8ccccc8c8c(-c9ccccc9)cccc87)cc56)n4)ccc23)cc1 ; c1ccc(-c2nc(-c3ccc4c(c3)oc3ccc(-n5c6ccccc6c6cccc(-c7ccccc7)c65)cc34)nc(-c3ccc4c(c3)sc3c(-c5ccccc5)cccc34)n2)cc1 ; c1ccc(-c2cccc(-c3cccc4sc5cc(-c6nc(-c7ccccc7)nc(-c7ccc8c(c7)oc7ccc(-n9c%10ccccc%10c%10ccccc%109)cc78)n6)ccc5c34)c2)cc1]

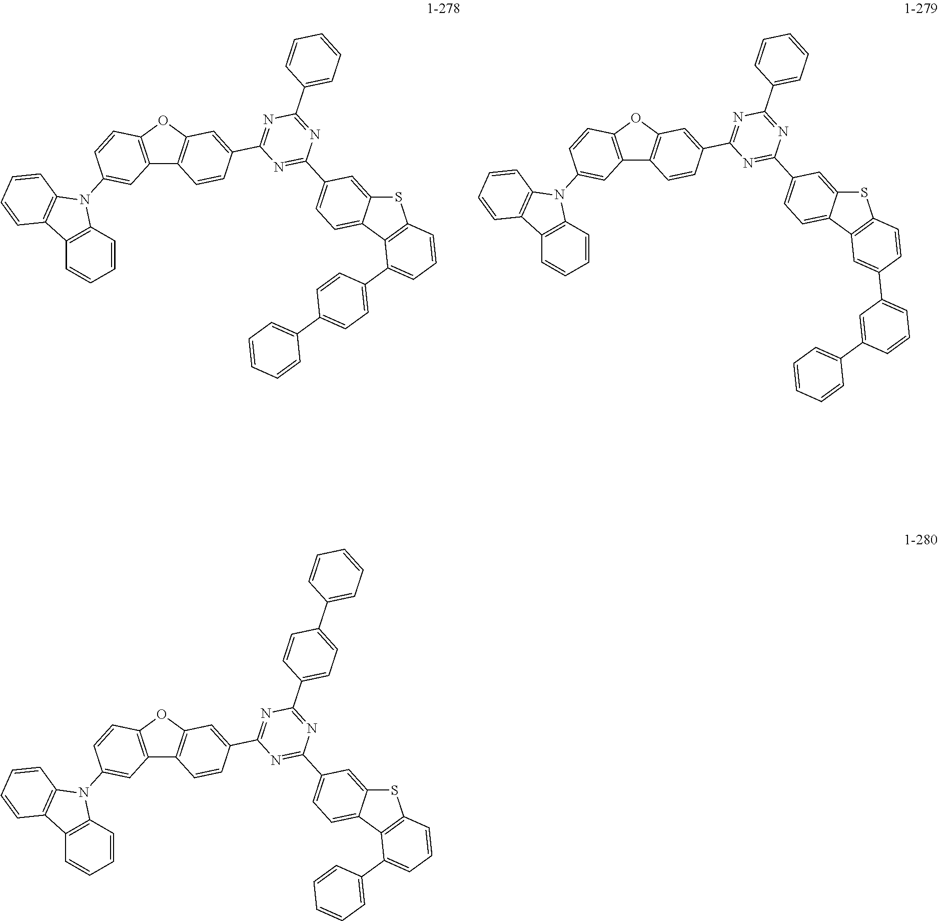 [SMILES: c1ccc(-c2ccc(-c3cccc4sc5cc(-c6nc(-c7ccccc7)nc(-c7ccc8c(c7)oc7ccc(-n9c%10ccccc%10c%10ccccc%109)cc78)n6)ccc5c34)cc2)cc1 ; c1ccc(-c2cccc(-c3ccc4sc5cc(-c6nc(-c7ccccc7)nc(-c7ccc8c(c7)oc7ccc(-n9c%10ccccc%10c%10ccccc%109)cc78)n6)ccc5c4c3)c2)cc1 ; c1ccc(-c2ccc(-c3nc(-c4ccc5c(c4)oc4ccc(-n6c7ccccc7c7ccccc76)cc45)nc(-c4ccc5c(c4)sc4cccc(-c6ccccc6)c45)n3)cc2)cc1]

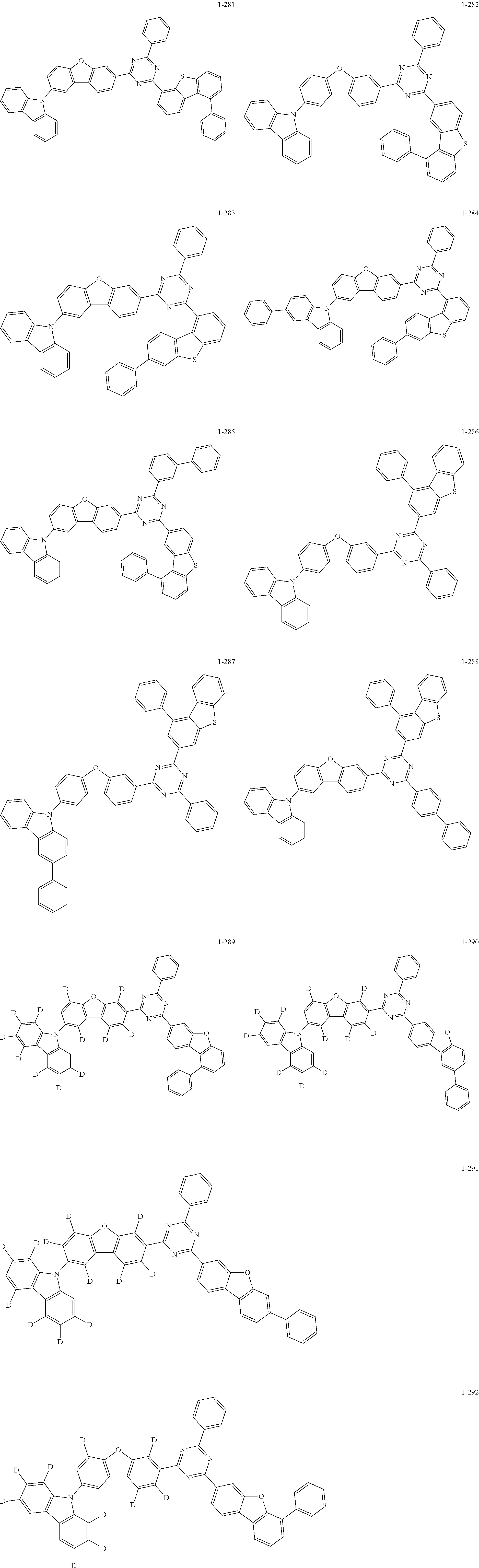

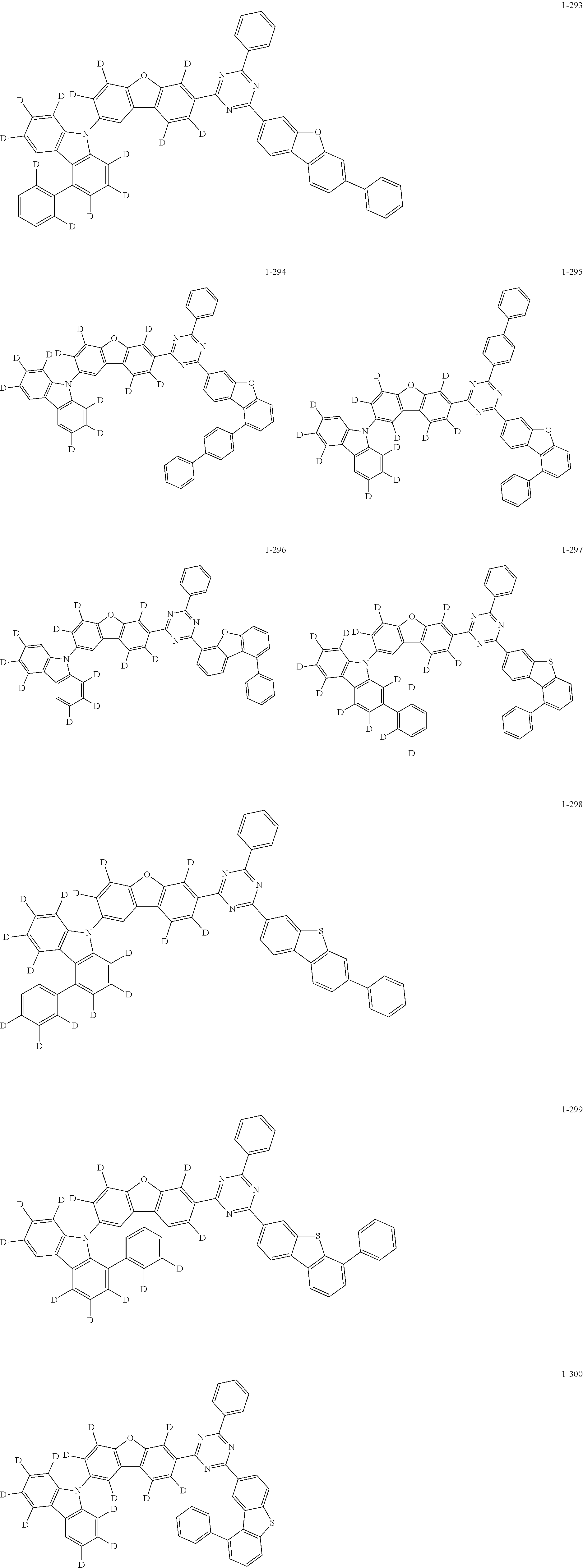

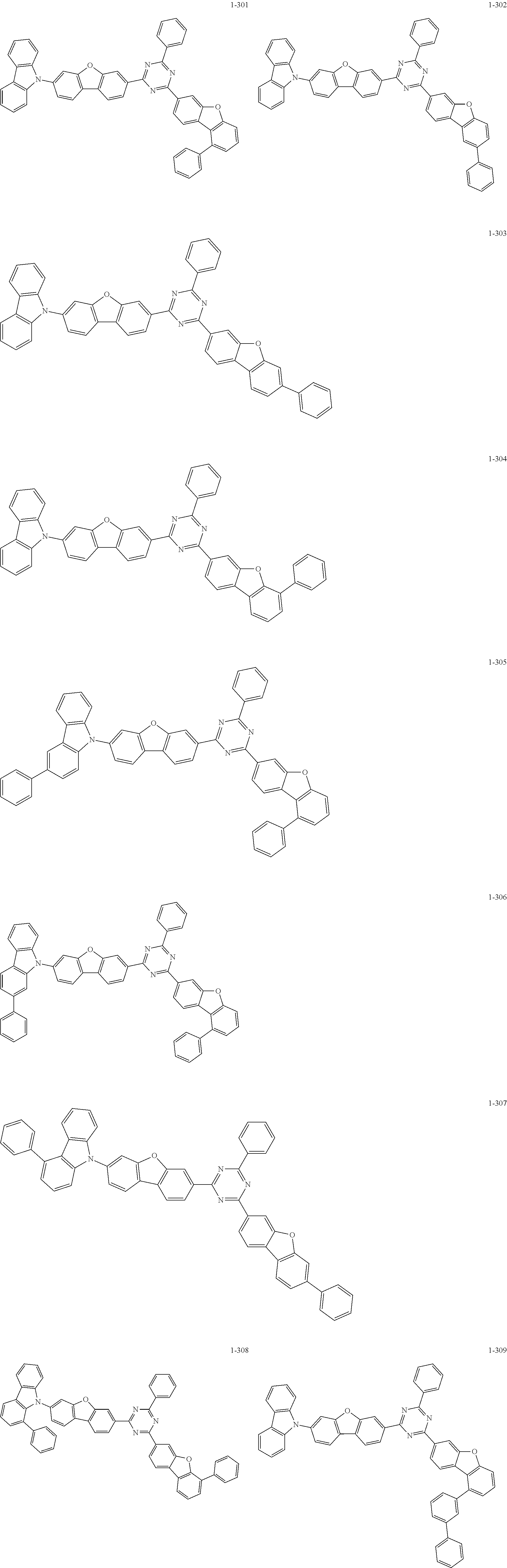

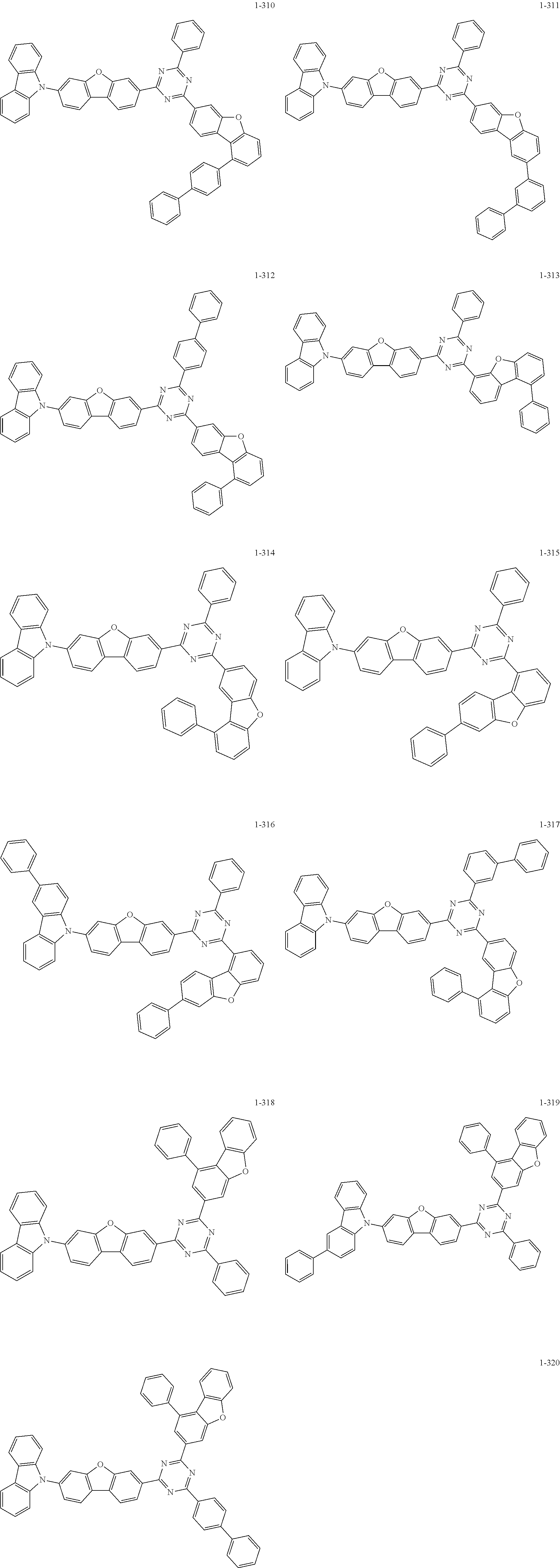

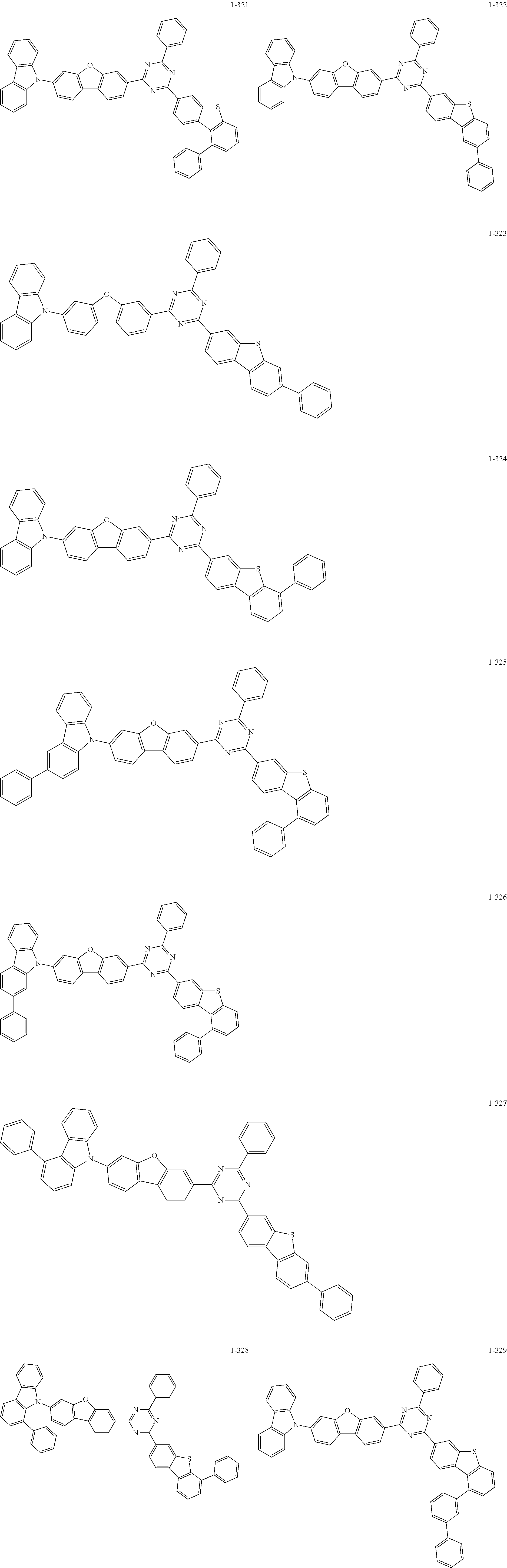

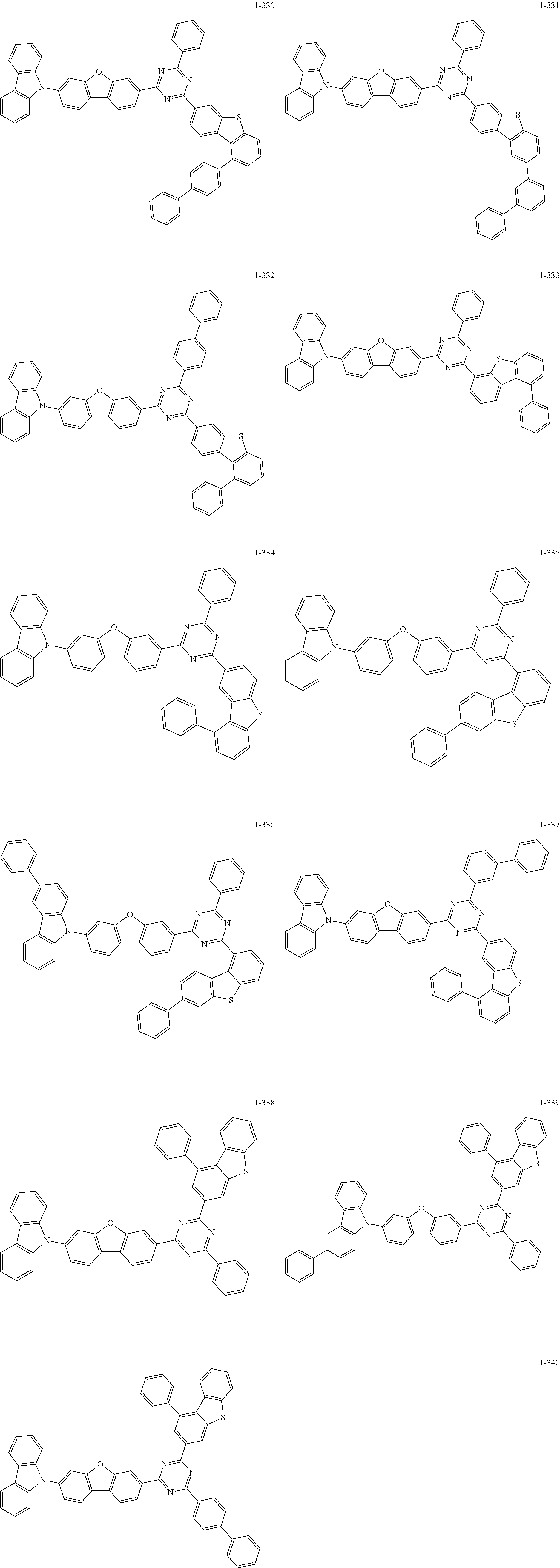

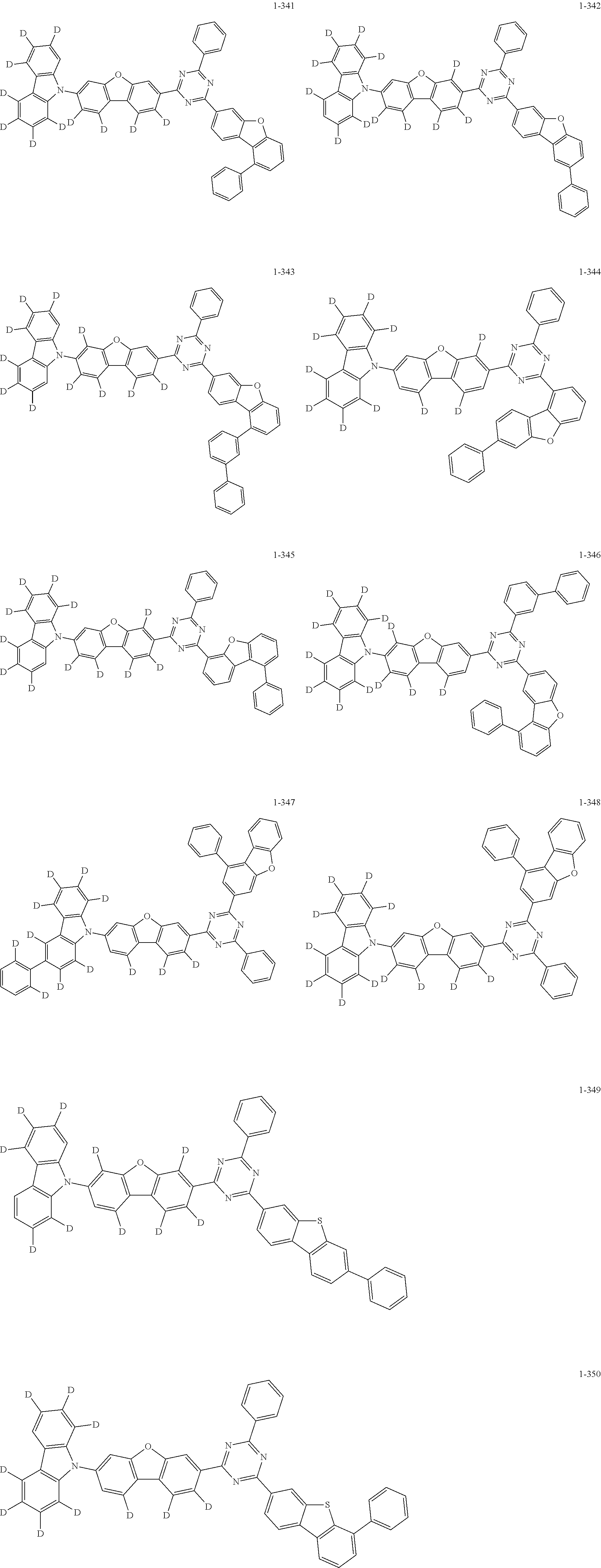

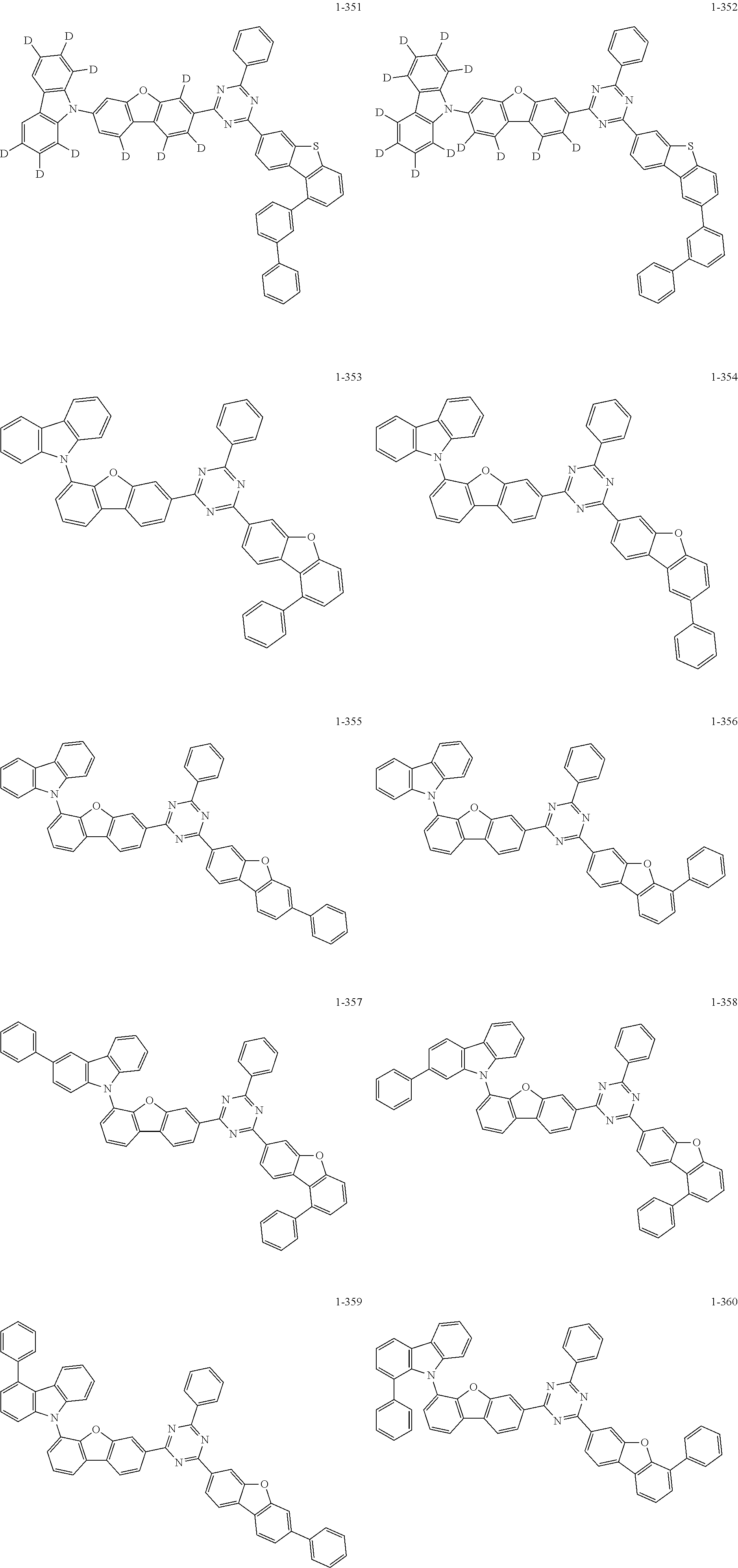

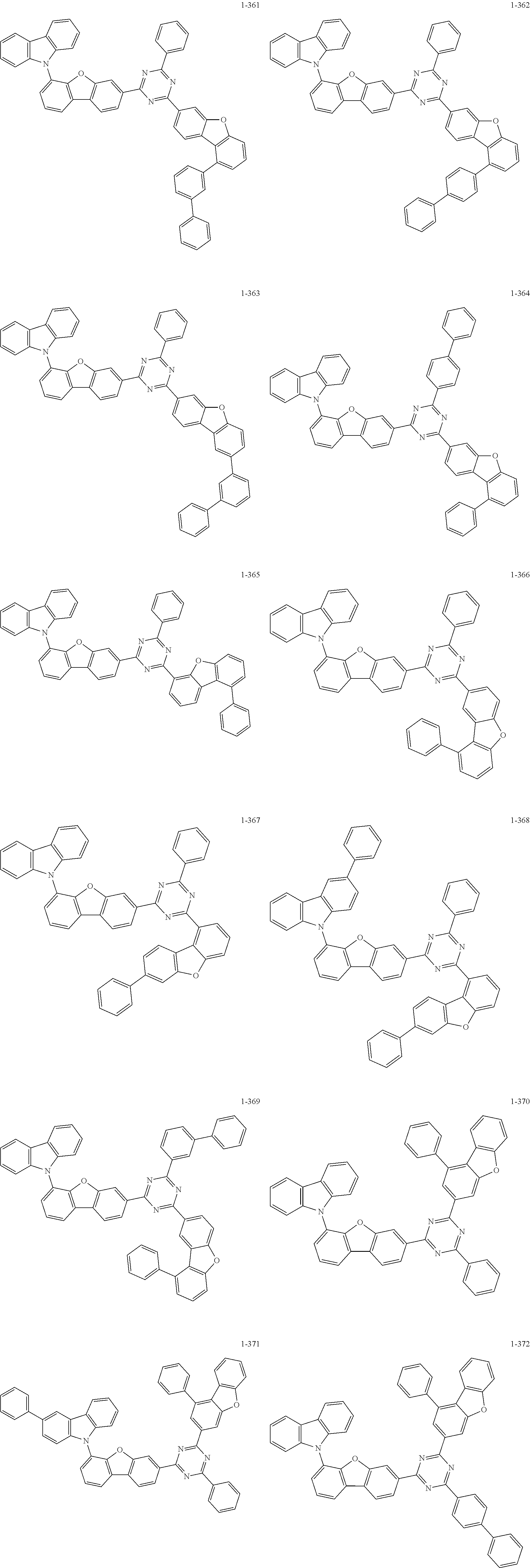

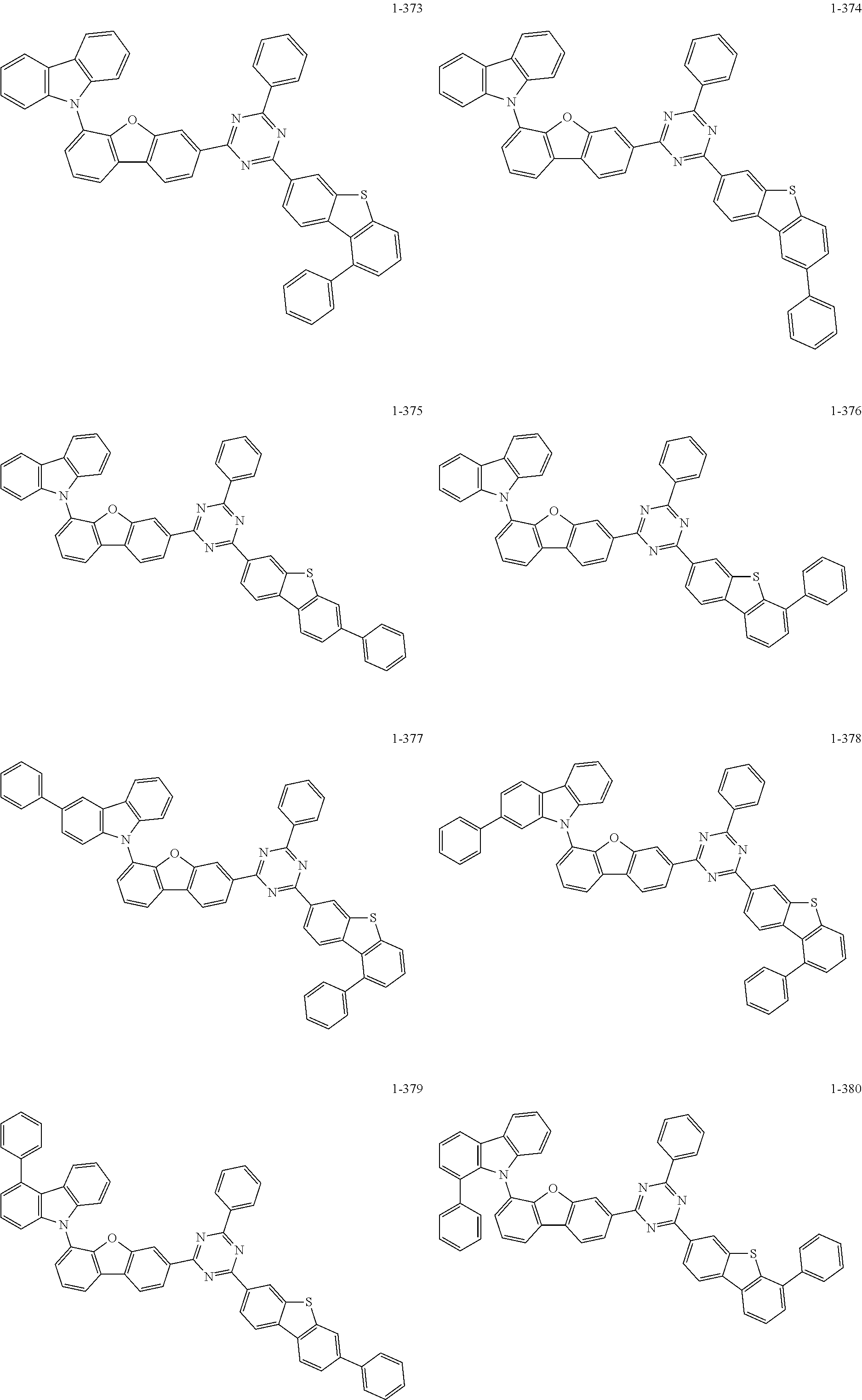

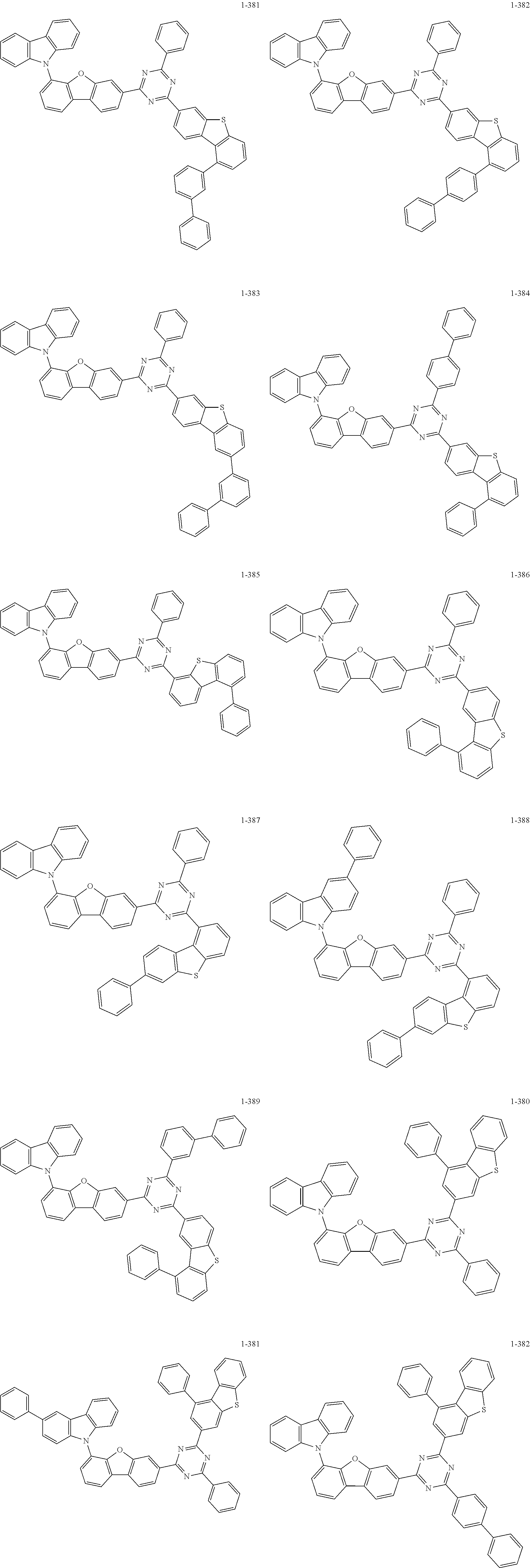

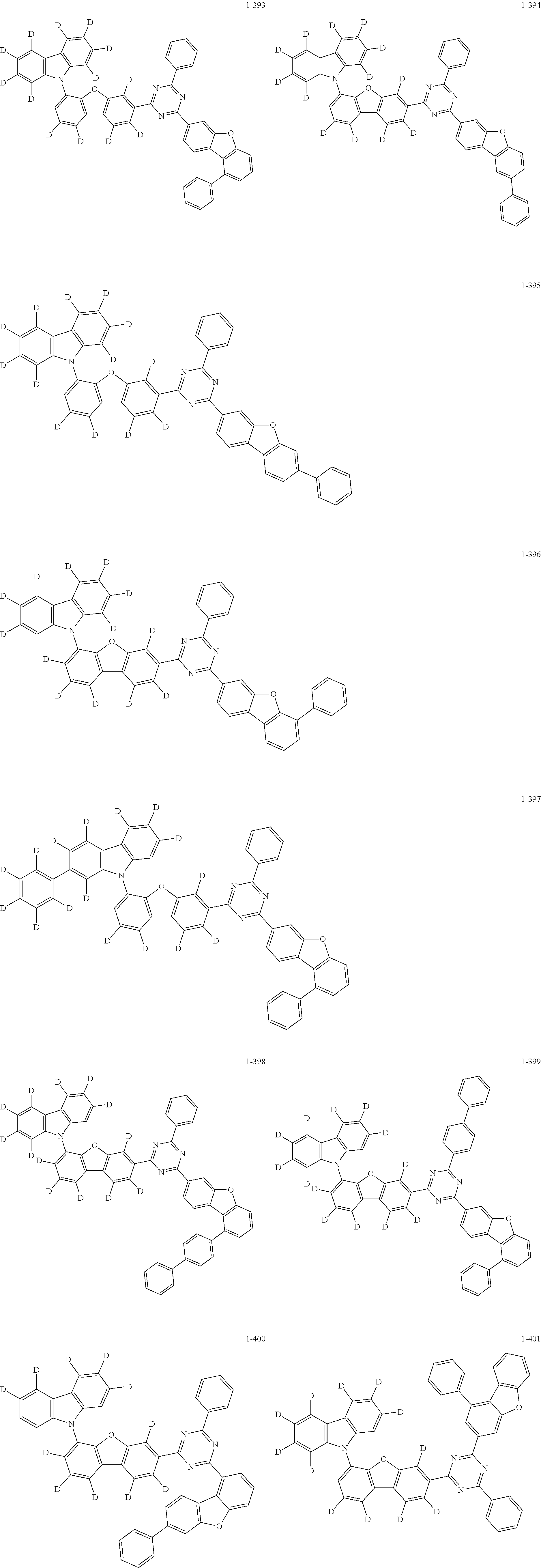

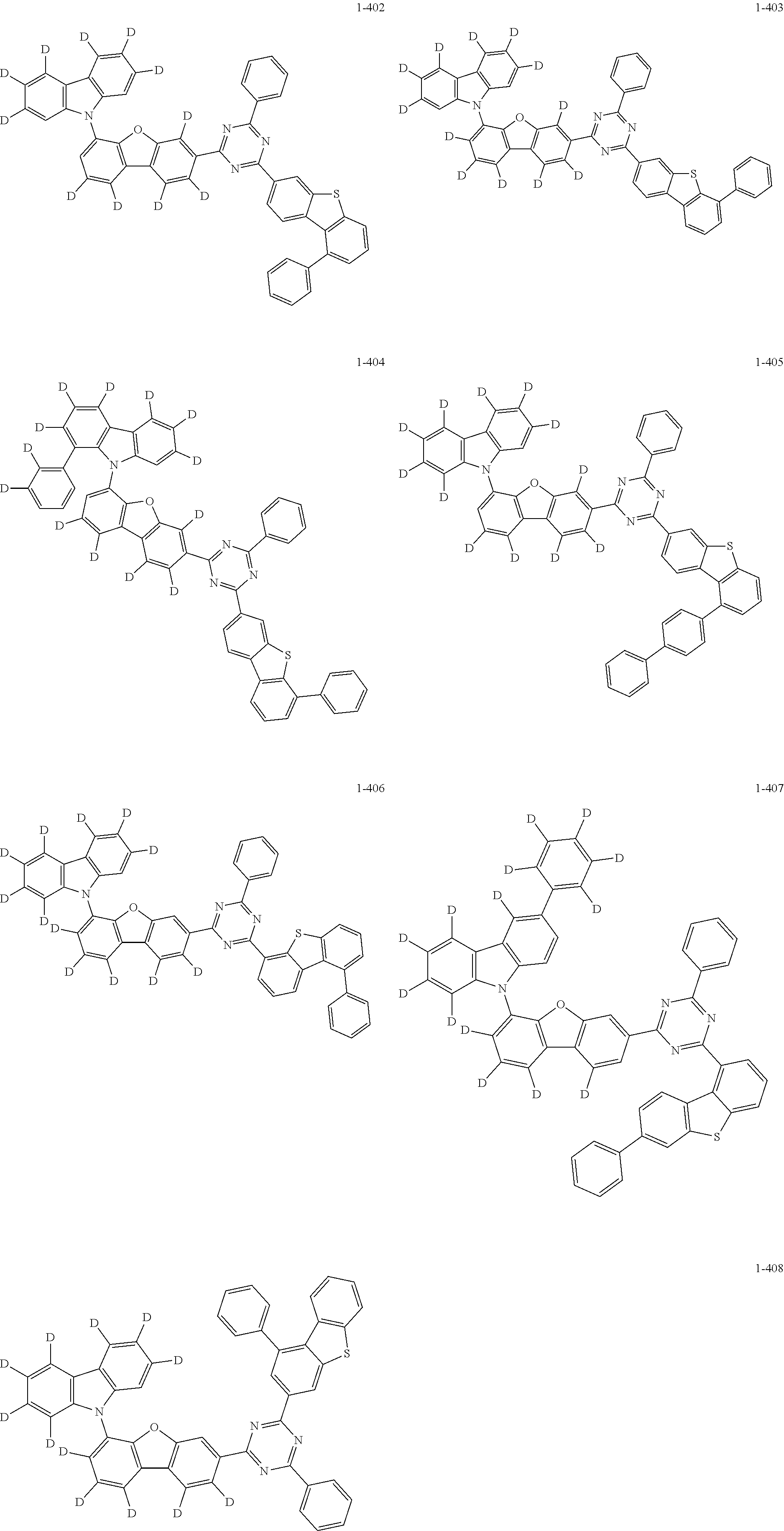

In addition, by introducing various substituents to the structure of Chemical Formula 1, compounds having unique properties of the introduced substituents may be synthesized. For example, by introducing substituents normally used for a hole injection layer material, a hole transport layer material, a light emitting layer material, an electron transport layer material and a charge generation layer material used for manufacturing an organic light emitting device to the core structure, materials satisfying conditions required for each organic material layer may be synthesized.

In addition, by introducing various substituents to the structure of Chemical Formula 1, the energy bandgap may be finely controlled, and meanwhile, properties at interfaces between organic materials may be enhanced, and material applications may become diverse.

Another embodiment of the present disclosure provides an organic light emitting device including the heterocyclic compound represented by Chemical Formula 1. The “organic light emitting device” may be expressed in terms such as an “organic light emitting diode”, an “OLED”, an “OLED device” and an “organic electroluminescent device”.

One embodiment of the present application provides an organic light emitting device including: a first electrode; a second electrode provided opposite to the first electrode; and one or more organic material layers provided between the first electrode and the second electrode, wherein one or more layers of the organic material layers include the heterocyclic compound represented by Chemical Formula 1.

In one embodiment of the present application, the first electrode may be a positive electrode, and the second electrode may be a negative electrode.

In another embodiment, the first electrode may be a negative electrode, and the second electrode may be a positive electrode.

In one embodiment of the present application, the organic light emitting device may be a blue organic light emitting device, and the heterocyclic compound according to Chemical Formula 1 may be used as a material of the blue organic light emitting device.

In one embodiment of the present application, the organic light emitting device may be a green organic light emitting device, and the heterocyclic compound represented by Chemical Formula 1 may be used as a material of the green organic light emitting device.

In one embodiment of the present application, the organic light emitting device may be a red organic light emitting device, and the heterocyclic compound represented by Chemical Formula 1 may be used as a material of the red organic light emitting device.

In one embodiment of the present application, the organic light emitting device may be a blue organic light emitting device, and the heterocyclic compound according to Chemical Formula 1 may be used as a light emitting layer material of the blue organic light emitting device.

In one embodiment of the present application, the organic light emitting device may be a green organic light emitting device, and the heterocyclic compound represented by Chemical Formula 1 may be used as a light emitting layer material of the green organic light emitting device.

In one embodiment of the present application, the organic light emitting device may be a red organic light emitting device, and the heterocyclic compound represented by Chemical Formula 1 may be used as a light emitting layer material of the red organic light emitting device.

Specific descriptions on the heterocyclic compound represented by Chemical Formula 1 are the same as the descriptions provided above.

The organic light emitting device of the present disclosure may be manufactured using common organic light emitting device manufacturing methods and materials except that one or more organic material layers are formed using the heterocyclic compound described above.

The heterocyclic compound may be formed into the organic material layer using a solution coating method as well as a vacuum deposition method when the organic light emitting device is manufactured. Herein, the solution coating method means spin coating, dip coating, inkjet printing, screen printing, a spray method, roll coating and the like, but is not limited thereto.

The organic material layer of the organic light emitting device of the present disclosure may be formed in a single layer structure, but may also be formed in a multilayer structure in which two or more organic material layers are laminated. For example, the organic light emitting device of the present disclosure may have a structure including a hole injection layer, a hole transport layer, a light emitting layer, an electron transport layer, an electron injection layer and the like as the organic material layer. However, the structure of the organic light emitting device is not limited thereto, and may include a smaller number of organic material layers.

In the organic light emitting device of the present disclosure, the organic material layer includes a light emitting layer, and the light emitting layer may include the heterocyclic compound represented by Chemical Formula 1.

Particularly, in the heterocyclic compound represented by Chemical Formula 1, the dibenzofuran parent structure

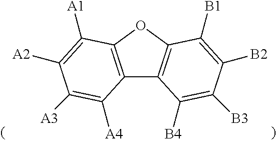

forms a resonance structure, and accordingly, the No. 2 and No. 4 carbon sites of the benzene ring included in the parent structure are negatively charged, and the No. 1 and No. 3 carbon sites become relatively electron deficient. Herein, when bonding an electron transport moiety to the No. 1 and No. 3 carbon sites that are relatively electron deficient and bonding a hole transport moiety to the No. 2 and No. 4 carbon sites that are relatively electron abundant, the transport speed of electrons in the molecule becomes faster. Accordingly, when a triazine substituent is introduced to the relatively electron-deficient No. 1 and No. 3 carbon sites of the benzene ring in the dibenzofuran parent structure as in the heterocyclic compound represented by Chemical Formula 1 according to the present disclosure, the role of the triazine substituent is strengthened compared to when a triazine substituent is introduced to the No. 2 and No. 4 carbon sites of the benzene ring.

In addition, the heterocyclic compound represented by Chemical Formula 1 according to the present disclosure further includes a dibenzofuran or dibenzothiophene substituent in the triazine substituent linked to the dibenzofuran parent structure, and further includes an aryl group or a heteroaryl group in the dibenzofuran or dibenzothiophene substituent, and accordingly, electron transport may be strengthened through expanding aromaticity of the LUMO (lowest unoccupied molecular orbital) level and the resonance structure. Particularly, when a heterocyclic substituent bonds to the dibenzofuran or dibenzothiophene substituent, the heterocyclic substituent includes an atom having higher electronegativity than carbon, and a property of donating electrons to the triazine substituent becomes weak compared to an aryl substituent. Accordingly, aromaticity of the LUMO becomes more superior when an aryl substituent bonds to the dibenzofuran or dibenzothiophene substituent compared to when a heterocyclic substituent bonds to the dibenzofuran or dibenzothiophene substituent, and electron transport in the heterocyclic compound represented by Chemical Formula 1 becomes smoother. When the heterocyclic compound represented by Chemical Formula 1 is used in an organic light emitting device together with the heterocyclic compound according to Chemical Formula 2 or 3 according to the present disclosure, there is an effect that intermolecular electron transport occurs more smoothly.

Furthermore, when substituting all hydrogens with deuterium in the structure of the heterocyclic compound represented by Chemical Formula 1, synthesis yield and deuterium substitution rate may decrease, and process efficiency may decrease since D6-benzene with a relatively high unit price is used in excess. Accordingly, intensively substituting hydrogens in the portion corresponding to the HOMO of the heterocyclic compound represented by the Chemical Formula 1 with deuterium may increase synthesis yield and deuterium substitution rate while using a relatively small amount of D6-benzene.

Accordingly, by using the compound represented by Chemical Formula 1 in the organic material layer, it is possible to lower a driving voltage of an organic light emitting device, enhance light emission efficiency, and enhance lifetime properties of an organic light emitting device due to thermal stability of the compound.

In the organic light emitting device of the present disclosure, the organic material layer includes a light emitting layer, and the light emitting layer may include the heterocyclic compound of Chemical Formula 1.

In the organic light emitting device of the present disclosure, the organic material layer includes a light emitting layer, and the light emitting layer may include the heterocyclic compound of Chemical Formula 1 as a light emitting layer host.

In another embodiment of the present disclosure, the organic light emitting device may further include, one, two or more layers selected from the group consisting of a light emitting layer, a hole injection layer, a hole transport layer, a hole transport auxiliary layer, an electron injection layer, an electron transport layer, an electron blocking layer and a hole blocking layer.

In one embodiment of the present disclosure, the organic light emitting device may include one or more organic material layers, the organic material layer may include a hole transport layer, and the hole transport layer may include the heterocyclic compound represented by Chemical Formula 1.

In one embodiment of the present disclosure, the organic light emitting device may include one or more organic material layers, the organic material layer may include a hole transport auxiliary layer, and the hole transport auxiliary layer may include the heterocyclic compound represented by Chemical Formula 1.

In one embodiment of the present disclosure, the organic material layer includes the heterocyclic compound represented by Chemical Formula 1, and a phosphorescent dopant may be used therewith.

As the phosphorescent dopant material, those known in the art may be used. For example, phosphorescent dopant materials represented by LL′MX′, LL′L″M, LMX′X″, L₂MX′ and L₃M may be used, however, the scope of the present disclosure is not limited by these examples.

M may be iridium, platinum, osmium or the like.

L is an anionic bidentate ligand coordinated to M by sp² carbon and heteroatom, and X may function to trap electrons or holes. Nonlimiting examples of L, L′ and L″ may include 2-(1-naphthyl)benzoxazole, 2-phenylbenzoxazole, 2-phenylbenzothiazole, 7,8-benzoquinoline, phenylpyridine, benzothiophenylpyridine, 3-methoxy-2-phenylpyridine, thiophenylpyridine, tolylpyridine and the like. Nonlimiting examples of X′ and X″ may include acetylacetonate (acac), hexafluoroacetylacetonate, salicylidene, picolinate, 8-hydroxyquinolinate and the like.

Specific examples of the phosphorescent dopant are shown below, however, the phosphorescent dopant is not limited to these examples:

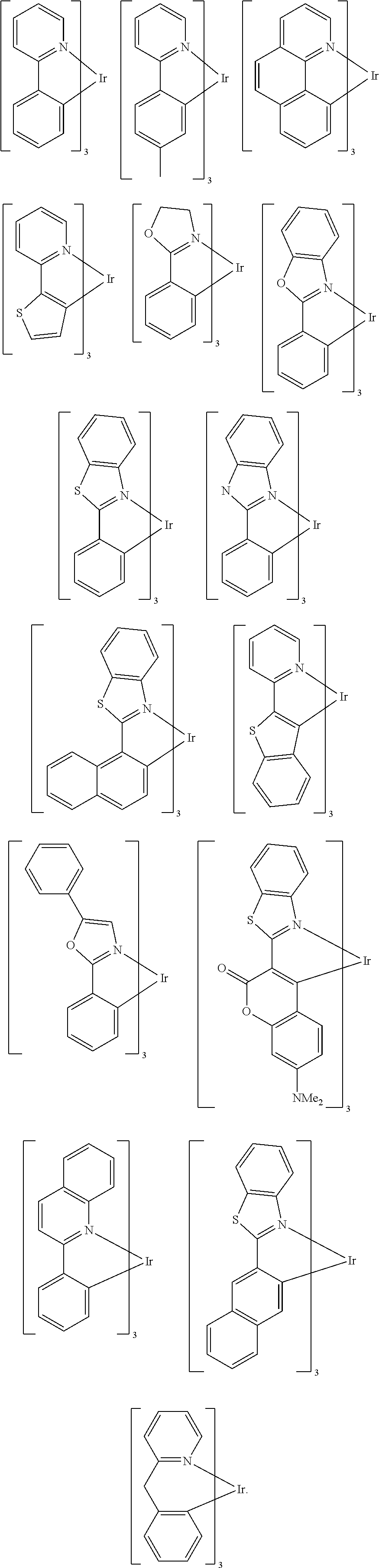

In one embodiment of the present disclosure, the organic material layer includes the heterocyclic compound represented by Chemical Formula 1, and an iridium-based dopant may be used therewith.

In one embodiment of the present disclosure, as the iridium-based dopant, (piq)₂(Ir)(acac), a red phosphorescent dopant, may be used.

In one embodiment of the present disclosure, as the iridium-based dopant, Ir(ppy)₃, a green phosphorescent dopant, may be used.

In one embodiment of the present disclosure, a content of the dopant may be from 1% to 15%, preferably from 2% to 10% and more preferably from 3% to 7% based on the total weight of the light emitting layer.

In the organic light emitting device according to one embodiment of the present disclosure, the organic material layer includes a hole transport layer or a hole transport auxiliary layer, and the hole transport layer or the hole transport auxiliary layer may include the heterocyclic compound represented by Chemical Formula 1.

In the organic light emitting device according to another embodiment of the present disclosure, the organic material layer includes an electron injection layer or an electron transport layer, and the electron injection layer or the electron transport layer may include the heterocyclic compound represented by Chemical Formula 1.

In the organic light emitting device according to another embodiment of the present disclosure, the organic material layer includes an electron blocking layer or a hole blocking layer, and the electron blocking layer or the hole blocking layer may include the heterocyclic compound represented by Chemical Formula 1.

In the organic light emitting device according to another embodiment of the present disclosure, the organic material layer includes an electron transport layer, a light emitting layer or a hole blocking layer, and the electron transport layer, the light emitting layer or the hole blocking layer may include the heterocyclic compound represented by Chemical Formula 1.

In the organic light emitting device according to another embodiment of the present disclosure, the organic material layer includes a light emitting layer, and the light emitting layer may include the heterocyclic compound represented by Chemical Formula 1.

In the organic light emitting device according to another embodiment of the present disclosure, the organic material layer includes a light emitting layer, the light emitting layer includes a host material, and the host material may include the heterocyclic compound represented by Chemical Formula 1.

In the organic light emitting device according to still another embodiment, the light emitting layer may include two or more host materials, and at least one of the host materials may include the heterocyclic compound represented by Chemical Formula 1.

In the organic light emitting device according to still another embodiment, two or more host materials may be pre-mixed and used in the light emitting layer, and at least one of the two or more host materials may include the heterocyclic compound represented by Chemical Formula 1.

The pre-mixing means, before depositing the two or more host materials on the organic material layer, mixing the materials first in one source of supply.

In the organic light emitting device according to one embodiment of the present application, an organic light emitting device wherein the organic material layer including the heterocyclic compound represented by Chemical Formula 1 further includes a heterocyclic compound represented by the following Chemical Formula 2 or 3 is provided.

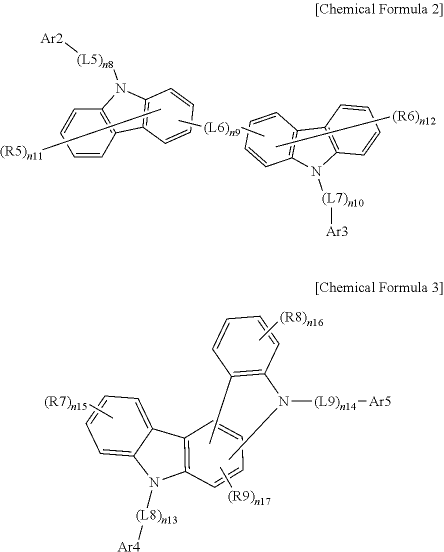

In Chemical Formulae 2 and 3,
L5 to L9 are the same as or different from each other, and each independently a single bond; a substituted or unsubstituted C6 to C60 arylene group; or a substituted or unsubstituted C2 to C60 heteroarylene group,
Ar2 to Ar5 are the same as or different from each other, and each independently a substituted or unsubstituted C6 to C60 aryl group; or a substituted or unsubstituted C2 to C60 heteroaryl group,
R5 to R9 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102 and —SiR101R102R103, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
n8 to n10, n13 and n14 are the same as or different from each other, and each independently an integer of 0 to 3,
n11 and n12 are an integer of 0 to 7,
n15 and n16 are an integer of 0 to 4, and
n17 is an integer of 0 to 2.

When the heterocyclic compound of Chemical Formula 1 and the heterocyclic compound of Chemical Formula 2 or Chemical Formula 3 are included in the organic material layer of the organic light emitting device, effects of more superior efficiency and lifetime are obtained. Such a result may lead to a forecast that an exciplex phenomenon occurs when the two compounds are included at the same time.

The exciplex phenomenon is a phenomenon of releasing energy having sizes of a donor (p-host) HOMO level and an acceptor (n-host) LUMO level due to electron exchanges between two molecules. When the exciplex phenomenon occurs between two molecules, reverse intersystem crossing (RISC) occurs, and as a result, internal quantum efficiency of fluorescence may increase up to 100%. When a donor (p-host) having a favorable hole transport ability and an acceptor (n-host) having a favorable electron transport ability are used as a host of a light emitting layer, holes are injected to the p-host and electrons are injected to the n-host, and therefore, a driving voltage may be lowered, which resultantly helps with enhancement in the lifetime.

In the heterocyclic compound represented by Chemical Formula 1, the dibenzofuran parent structure

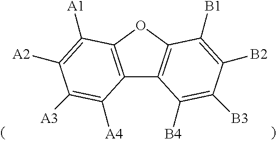

forms a resonance structure, and accordingly, the No. 2 and No. 4 carbon sites of the benzene ring included in the parent structure are negatively charged, and the No. 1 and No. 3 carbon sites become relatively electron deficient. Herein, when bonding an electron transport moiety to the No. 1 and No. 3 carbon sites that are relatively electron deficient and bonding a hole transport moiety to the No. 2 and No. 4 carbon sites that are relatively electron abundant, the transport speed of electrons in the molecule becomes faster. Accordingly, when a triazine substituent is introduced to the relatively electron-deficient No. 1 and No. 3 carbon sites of the benzene ring in the dibenzofuran parent structure as in the heterocyclic compound represented by Chemical Formula 1 according to the present disclosure, the role of the triazine substituent is strengthened compared to when a triazine substituent is introduced to the No. 2 and No. 4 carbon sites of the benzene ring.

In addition, the heterocyclic compound represented by Chemical Formula 1 according to the present disclosure further includes a dibenzofuran or dibenzothiophene substituent in the triazine substituent linked to the dibenzofuran parent structure, and further includes an aryl group or a heteroaryl group in the dibenzofuran or dibenzothiophene substituent, and accordingly, electron transport may be strengthened through expanding aromaticity of the LUMO (lowest unoccupied molecular orbital) level and the resonance structure. Particularly, when a heterocyclic substituent dibenzofuran or bonds to the dibenzothiophene substituent, the heterocyclic substituent includes an atom having higher electronegativity than carbon, and a property of donating electrons to the triazine substituent becomes weak compared to an aryl substituent. Accordingly, aromaticity of the LUMO becomes more superior when an aryl substituent bonds to the dibenzofuran or dibenzothiophene substituent compared to when a heterocyclic substituent bonds to the dibenzofuran or dibenzothiophene substituent, and electron transport in the heterocyclic compound represented by Chemical Formula 1 becomes smoother. When the heterocyclic compound represented by Chemical Formula 1 is used in an organic light emitting device together with the heterocyclic compound according to Chemical Formula 2 or 3 according to the present disclosure, there is an effect that intermolecular electron transport occurs more smoothly.

Furthermore, when substituting all hydrogens with deuterium in the structure of the heterocyclic compound represented by Chemical Formula 1, synthesis yield and deuterium substitution rate may decrease, and process efficiency may decrease since D6-benzene with a relatively high unit price is used in excess. Accordingly, intensively substituting hydrogens in the portion corresponding to the HOMO of the heterocyclic compound represented by the Chemical Formula 1 with deuterium may increase synthesis yield and deuterium substitution rate while using a relatively small amount of D6-benzene.

Accordingly, by using the compound represented by Chemical Formula 1 in the organic material layer, it is possible to lower a driving voltage of an organic light emitting device, enhance light emission efficiency, and enhance lifetime properties of an organic light emitting device due to thermal stability of the compound.

In one embodiment of the present application, L5 to L9 are the same as or different from each other, and may be each independently a single bond; a substituted or unsubstituted C6 to C40 arylene group; or a substituted or unsubstituted C2 to C40 heteroarylene group.

In one embodiment of the present application, L5 to L9 are the same as or different from each other, and may be each independently a single bond; a substituted or unsubstituted C6 to C30 arylene group; or a substituted or unsubstituted C2 to C30 heteroarylene group.

In one embodiment of the present application, L5 to L9 are the same as or different from each other, and may be each independently a single bond; a substituted or unsubstituted C6 to C20 arylene group; or a substituted or unsubstituted C2 to C20 heteroarylene group.

In one embodiment of the present application, L5 to L9 are the same as or different from each other, and may be each independently a single bond; a substituted or unsubstituted C6 to C10 arylene group; or a substituted or unsubstituted C2 to C10 heteroarylene group.

In one embodiment of the present application, L5 to L9 are the same as or different from each other, and may be each independently a single bond; a substituted or unsubstituted phenylene group; a substituted or unsubstituted biphenylene group; a substituted or unsubstituted naphthylene group; or a substituted or unsubstituted dibenzofuranylene group.

In one embodiment of the present application, Ar2 to Ar5 are the same as or different from each other, and may be each independently a substituted or unsubstituted C6 to C40 aryl group; or a substituted or unsubstituted C2 to C40 heteroaryl group.

In one embodiment of the present application, Ar2 to Ar5 are the same as or different from each other, and may be each independently a substituted or unsubstituted C6 to C30 aryl group; or a substituted or unsubstituted C2 to C30 heteroaryl group.

In one embodiment of the present application, Ar2 to Ar5 are the same as or different from each other, and may be each independently a substituted or unsubstituted C6 to C20 aryl group; or a substituted or unsubstituted C2 to C20 heteroaryl group.

In one embodiment of the present application, Ar2 to Ar5 are the same as or different from each other, and may be each independently a substituted or unsubstituted C6 to C10 aryl group; or a substituted or unsubstituted C2 to C10 heteroaryl group.

In one embodiment of the present application, Ar2 to Ar5 are the same as or different from each other, and may be each independently a substituted or unsubstituted phenyl group; a substituted or unsubstituted biphenyl group; a substituted or unsubstituted naphthyl group; a substituted or unsubstituted terphenyl group; a substituted or unsubstituted fluorenyl group; a substituted or unsubstituted 9,9-dimethylfluorenyl group; a substituted or unsubstituted anthracenyl group; a substituted or unsubstituted pyrenyl group; a substituted or unsubstituted chrysenyl group; a substituted or unsubstituted fluoranthenyl group; a substituted or unsubstituted 9,9-diphenylfluorenyl group; a substituted or unsubstituted phenanthrenyl group; a substituted or unsubstituted dibenzofuran group; a substituted or unsubstituted dibenzothiophene group; a substituted or unsubstituted spirobifluorenyl group; a substituted or unsubstituted triphenylenyl group; or a substituted or unsubstituted tetraphenylsilane group.

In one embodiment of the present application, R5 to R9 are the same as or different from each other, and each independently selected from the group consisting of hydrogen; deuterium; halogen; a cyano group; a substituted or unsubstituted C1 to C40 alkyl group; a substituted or unsubstituted C2 to C40 alkenyl group; a substituted or unsubstituted C2 to C40 alkynyl group; a substituted or unsubstituted C1 to C40 alkoxy group; a substituted or unsubstituted C3 to C40 cycloalkyl group; a substituted or unsubstituted C2 to C40 heterocycloalkyl group; a substituted or unsubstituted C6 to C40 aryl group; a substituted or unsubstituted C2 to C40 heteroaryl group; —P(═O)R101R102 and —SiR101R102R103, or two or more groups adjacent to each other bond to each other to form a substituted or unsubstituted C6 to C40 aromatic hydrocarbon ring or a substituted or unsubstituted C2 to C40 heteroring, and R101, R102 and R103 are the same as or different from each other and may be each independently a substituted or unsubstituted C1 to C40 alkyl group; a substituted or unsubstituted C6 to C40 aryl group; or a substituted or unsubstituted C2 to C40 heteroaryl group.

In one embodiment of the present application, R5 to R9 are the same as or different from each other, and each independently selected from the group consisting of hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R101R102 and —SiR101R102R103, or two or more groups adjacent to each other bond to each other to form a substituted or unsubstituted C6 to C30 aromatic hydrocarbon ring or a substituted or unsubstituted C2 to C30 heteroring, and R101, R102 and R103 are the same as or different from each other and may be each independently a substituted or unsubstituted C1 to C30 alkyl group; a substituted or unsubstituted C6 to C30 aryl group; or a substituted or unsubstituted C2 to C30 heteroaryl group.

In one embodiment of the present application, R5 to R9 are the same as or different from each other, and each independently selected from the group consisting of hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; and a substituted or unsubstituted C2 to C20 heteroaryl group, or two or more groups adjacent to each other may bond to each other to form a substituted or unsubstituted C6 to C20 aromatic hydrocarbon ring or a substituted or unsubstituted C2 to C20 heteroring.

In one embodiment of the present application, R5 to R9 are the same as or different from each other, and each independently selected from the group consisting of hydrogen; deuterium; halogen; a cyano group; a substituted or unsubstituted C1 to C10 alkyl group; a substituted or unsubstituted C2 to C10 alkenyl group; a substituted or unsubstituted C2 to C10 alkynyl group; a substituted or unsubstituted C1 to C10 alkoxy group; a substituted or unsubstituted C3 to C10 cycloalkyl group; a substituted or unsubstituted C2 to C10 heterocycloalkyl group; a substituted or unsubstituted C6 to C10 aryl group; and a substituted or unsubstituted C2 to C10 heteroaryl group, or two or more groups adjacent to each other may bond to each other to form a substituted or unsubstituted C6 to C10 aromatic hydrocarbon ring or a substituted or unsubstituted C2 to C10 heteroring.

In one embodiment of the present application, R5 to R9 are the same as or different from each other, and may be each independently hydrogen; deuterium; a substituted or unsubstituted phenyl group; a substituted or unsubstituted biphenyl group; a substituted or unsubstituted naphthyl group; or a substituted or unsubstituted terphenyl group.

In one embodiment of the present application, R5 to R9 are the same as or different from each other, and may be each independently hydrogen; or deuterium.

In one embodiment of the present application, Ar2 to Ar5 may be represented by any one of the following Structural Formula 3 or 4.

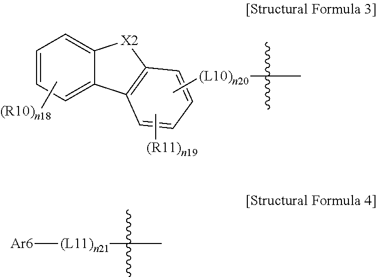

In Structural Formulae 3 and 4,
X2 is O, S or CRaRb,
L10 and L11 are the same as or different from each other, and each independently a single bond; a substituted or unsubstituted C6 to C60 arylene group; or a substituted or unsubstituted C2 to C60 heteroarylene group,
Ar6 is a substituted or unsubstituted C6 to C60 aryl group,
R10, R11, Ra and Rb are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102 and —SiR101R102R103, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
n18 is an integer of 0 to 4,
n19 is an integer of 0 to 3, and
n20 and n21 are the same as or different from each other, and each independently an integer of 0 to 3.

In one embodiment of the present application, X2 of Structural Formula 3 may be O.

In one embodiment of the present application, X2 of Structural Formula 3 may be S.

In one embodiment of the present application, X2 of Structural Formula 3 may be CRaRb.

In one embodiment of the present application, L10 and L11 are the same as or different from each other, and may be each independently a direct bond; a substituted or unsubstituted C6 to C40 arylene group; or a substituted or unsubstituted C2 to C40 heteroarylene group.

In one embodiment of the present application, L10 and L11 are the same as or different from each other, and may be each independently a direct bond; a substituted or unsubstituted C6 to C30 arylene group; or a substituted or unsubstituted C2 to C30 heteroarylene group.

In one embodiment of the present application, L10 and L11 are the same as or different from each other, and may be each independently a direct bond; a substituted or unsubstituted C6 to C20 arylene group; or a substituted or unsubstituted C2 to C20 heteroarylene group.

In one embodiment of the present application, L10 and L11 are the same as or different from each other, and may be each independently a direct bond; a substituted or unsubstituted C6 to C10 arylene group; or a substituted or unsubstituted C2 to C10 heteroarylene group.

In one embodiment of the present application, L10 and L11 are the same as or different from each other, and may be each independently a direct bond; a substituted or unsubstituted phenylene group; a substituted or unsubstituted biphenylene group; a substituted or unsubstituted naphthylene group; or a substituted or unsubstituted dibenzofuranylene group.

In one embodiment of the present application, Ar6 may be a substituted or unsubstituted C6 to C40 aryl group.

In one embodiment of the present application, Ar6 may be a substituted or unsubstituted C6 to C30 aryl group.

In one embodiment of the present application, Ar6 may be a substituted or unsubstituted C6 to C20 aryl group.

In one embodiment of the present application, Ar6 may be a substituted or unsubstituted C6 to C10 aryl group.

In one embodiment of the present application, Ar6 may be selected from among a substituted or unsubstituted phenyl group; a substituted or unsubstituted biphenyl group; a substituted or unsubstituted terphenyl group; a substituted or unsubstituted naphthyl group; a substituted or unsubstituted fluorenyl group; a substituted or unsubstituted phenanthrenyl group; a substituted or unsubstituted anthracenyl group; a substituted or unsubstituted fluoranthenyl group; a substituted or unsubstituted triphenylenyl group; a substituted or unsubstituted pyrenyl group; and a substituted or unsubstituted chrysenyl group.

In one embodiment of the present application, R10, R11, Ra and Rb are the same as or different from each other, and each independently selected from the group consisting of hydrogen; deuterium; halogen; a cyano group; a substituted or unsubstituted C1 to C40 alkyl group; a substituted or unsubstituted C2 to C40 alkenyl group; a substituted or unsubstituted C2 to C40 alkynyl group; a substituted or unsubstituted C1 to C40 alkoxy group; a substituted or unsubstituted C3 to C40 cycloalkyl group; a substituted or unsubstituted C2 to C40 heterocycloalkyl group; a substituted or unsubstituted C6 to C40 aryl group; a substituted or unsubstituted C2 to C40 heteroaryl group; —P(═O)R101R102 and —SiR101R102R103, or two or more groups adjacent to each other bond to each other to form a substituted or unsubstituted C6 to C40 aromatic hydrocarbon ring or a substituted or unsubstituted C2 to C40 heteroring, and R101, R102 and R103 are the same as or different from each other and may be each independently a substituted or unsubstituted C1 to C40 alkyl group; a substituted or unsubstituted C6 to C40 aryl group; or a substituted or unsubstituted C2 to C40 heteroaryl group.

In one embodiment of the present application, R10, R11, Ra and Rb are the same as or different from each other, and each independently selected from the group consisting of hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R101R102 and —SiR101R102R103, or two or more groups adjacent to each other bond to each other to form a substituted or unsubstituted C6 to C30 aromatic hydrocarbon ring or a substituted or unsubstituted C2 to C30 heteroring, an R101, R102 and R103 are the same as or different from each other and may be each independently a substituted or unsubstituted C1 to C30 alkyl group; a substituted or unsubstituted C6 to C30 aryl group; or a substituted or unsubstituted C2 to C30 heteroaryl group.

In one embodiment of the present application, R10, R11, Ra and Rb are the same as or different from each other, and each independently selected from the group consisting of hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; substituted or unsubstituted C2 to C20 heterocycloalkyl group; a substituted or unsubstituted C6 to C20 aryl group; and a substituted or unsubstituted C2 to C20 heteroaryl group, or two or more groups adjacent to each other may bond to each other to form a substituted or unsubstituted C6 to C20 aromatic hydrocarbon ring or a substituted or unsubstituted C2 to C20 heteroring.

In one embodiment of the present application, R10, R11, Ra and Rb are the same as or different from each other, and each independently selected from the group consisting of hydrogen; deuterium; halogen; a cyano group; a substituted or unsubstituted C1 to C10 alkyl group; a substituted or unsubstituted C2 to C10 alkenyl group; a substituted or unsubstituted C2 to C10 alkynyl group; a substituted or unsubstituted C1 to C10 alkoxy group; a substituted or unsubstituted C3 to C10 cycloalkyl group; a substituted or unsubstituted C2 to C10 heterocycloalkyl group; a substituted or unsubstituted C6 to C10 aryl group; and a substituted or unsubstituted C2 to C10 heteroaryl group, or two or more groups adjacent to each other may bond to each other to form a substituted or unsubstituted C6 to C10 aromatic hydrocarbon ring or a substituted or unsubstituted C2 to C10 heteroring.

In one embodiment of the present application, R10, R11, Ra and Rb are the same as or different from each other, and each independently hydrogen; deuterium; a substituted or unsubstituted methyl group; a substituted or unsubstituted phenyl group; a substituted or unsubstituted biphenyl group; a substituted or unsubstituted naphthyl group; a substituted or unsubstituted terphenyl group; a substituted or unsubstituted fluorenyl group; a substituted or unsubstituted 9,9-dimethylfluorenyl group; a substituted or unsubstituted anthracenyl group; a substituted or unsubstituted pyrenyl group; a substituted or unsubstituted chrysenyl group; a substituted or unsubstituted fluoranthenyl group; a substituted or unsubstituted 9,9-diphenylfluorenyl group; a substituted or unsubstituted phenanthrenyl group; a substituted or unsubstituted dibenzofuran group; a substituted or unsubstituted dibenzothiophene group; a substituted or unsubstituted spirobifluorenyl group; a substituted or unsubstituted triphenylenyl group; or a substituted or unsubstituted tetraphenylsilane group, or two or more groups adjacent to each other may bond to each other to form a substituted or unsubstituted spirobifluorenyl group.

In one embodiment of the present application, R10, R11, Ra and Rb are the same as or different from each other, and each independently hydrogen; deuterium; a substituted or unsubstituted methyl group; or a substituted or unsubstituted phenyl group, or two or more groups adjacent to each other may bond to each other to form a substituted or unsubstituted spirobifluorenyl group.

In one embodiment of the present application, the heterocyclic compound represented by Chemical Formula 3 may be represented by any one of the following Chemical Formulae 3-1 to 3-5.

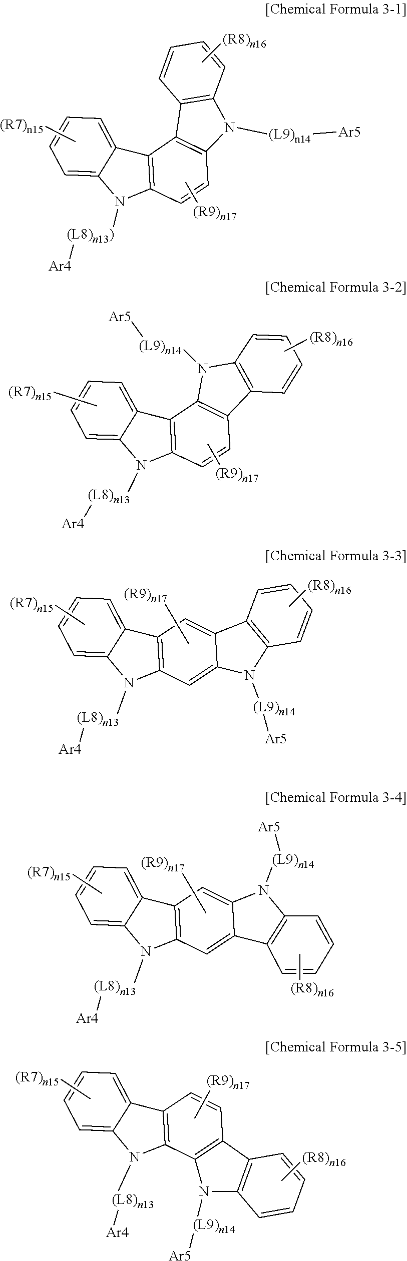

In Chemical Formulae 3-1 to 3-5,
R7 to R9, L8, L9, Ar4, Ar5 and n13 to n17 have the same definitions as in Chemical Formula 3.

In one embodiment of the present application, the heterocyclic compound represented by Chemical Formula 2 or 3 may not include deuterium as a substituent, or may have a deuterium content of, for example, greater than 08, 1% or greater, 10% or greater, 20% or greater, 30% or greater, 40% or greater or 50% or greater, and 100% or less, 90% or less, 80% or less, 70% or less or 60% or less with respect to the total number of hydrogen atoms and deuterium atoms.

In one embodiment of the present application, the heterocyclic compound represented by Chemical Formula 2 may not include deuterium, or may have a deuterium content of 1% to 100% with respect to the total number of hydrogen atoms and deuterium atoms.

In one embodiment of the present application, the heterocyclic compound represented by Chemical Formula 2 may not include deuterium, or may have a deuterium content of 10% to 100% with respect to the total number of hydrogen atoms and deuterium atoms.

In embodiment of the present application, the heterocyclic compound represented by Chemical Formula 2 may not include deuterium, or may have a deuterium content of 20% to 90% with respect to the total number of hydrogen atoms and deuterium atoms.

In one embodiment of the present application, the heterocyclic compound represented by Chemical Formula 2 may not include deuterium, or may have a deuterium content of 30% to 80% with respect to the total number of hydrogen atoms and deuterium atoms.

In one embodiment of the present application, the heterocyclic compound represented by Chemical Formula 2 may not include deuterium, or may have a deuterium content of 40% to 70% with respect to the total number of hydrogen atoms and deuterium atoms.

In one embodiment of the present application, the heterocyclic compound represented by Chemical Formula 2 may have a deuterium content of 0% or greater, 1% or greater, 5% or greater, 10% or greater, 15% or greater, 20% or greater, 25% or greater, 30% or greater, 35% or greater, 40% or greater, 45% or greater or 50% or greater, and 100% or less, 95% or less, 90% or less, 85% or less, 80% or less, 75% or less, 70% or less, 65% or less or 60% or less based on the total number of hydrogen atoms and deuterium atoms.

In one embodiment of the present application, the heterocyclic compound represented by Chemical Formula 3 may not include deuterium, or may have a deuterium content of 1% to 100% with respect to the total number of hydrogen atoms and deuterium atoms.

In one embodiment of the present application, the heterocyclic compound represented by Chemical Formula 3 may not include deuterium, or may have a deuterium content of 10% to 100% with respect to the total number of hydrogen atoms and deuterium atoms.

In one embodiment of the present application, the heterocyclic compound represented by Chemical Formula 3 may not include deuterium, or may have a deuterium content of 20% to 90% with respect to the total number of hydrogen atoms and deuterium atoms.

In one embodiment of the present application, the heterocyclic compound represented by Chemical Formula 3 may not include deuterium, or may have a deuterium content of 30% to 80% with respect to the total number of hydrogen atoms and deuterium atoms.

In one embodiment of the present application, the heterocyclic compound represented by Chemical Formula 3 may not include deuterium, or may have a deuterium content of 40% to 70% with respect to the total number of hydrogen atoms and deuterium atoms.

In one embodiment of the present application, the heterocyclic compound represented by Chemical Formula 3 may have a deuterium content of 0% or greater, 1% or greater, 5% or greater, 10% or greater, 15% or greater, 20% or greater, 25% or greater, 30% or greater, 35% or greater, 40% or greater, 45% or greater or 50% or greater, and 100% or less, 95% or less, 90% or less, 85% or less, 80% or less, 75% or less, 70% or less, 65% or less or 60% or less based on the total number of hydrogen atoms and deuterium atoms.

One embodiment of the present application provides a heterocyclic compound, wherein Chemical Formula 2 is represented by any one of the following compounds. In addition, in one embodiment of the present application, the following compounds are just one example, and the present application is not limited thereto and may include other compounds included in Chemical Formula 2 including additional substituents.

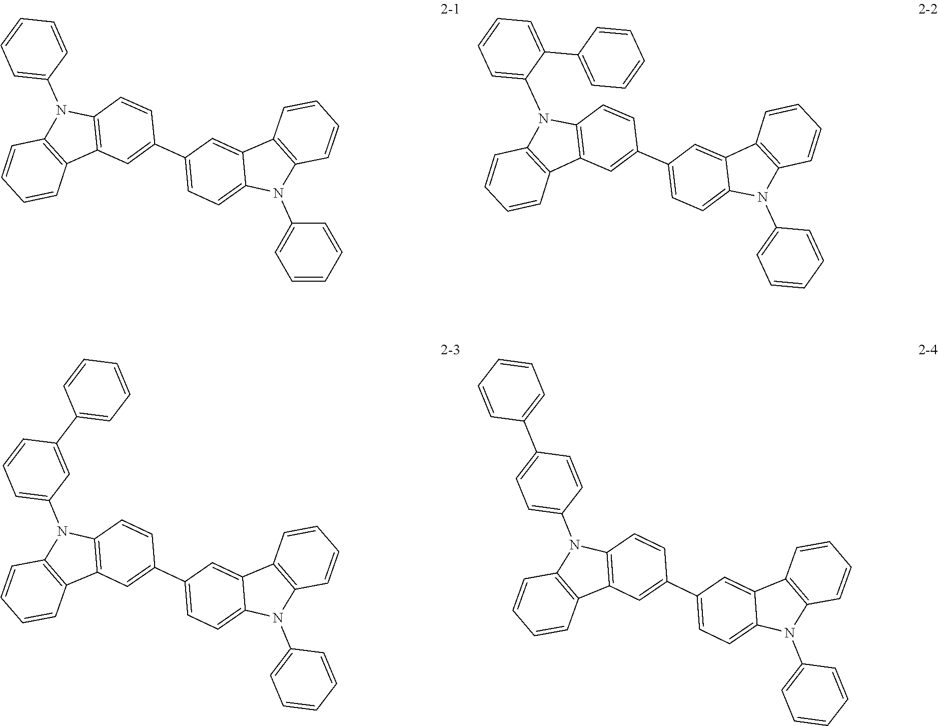 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1]

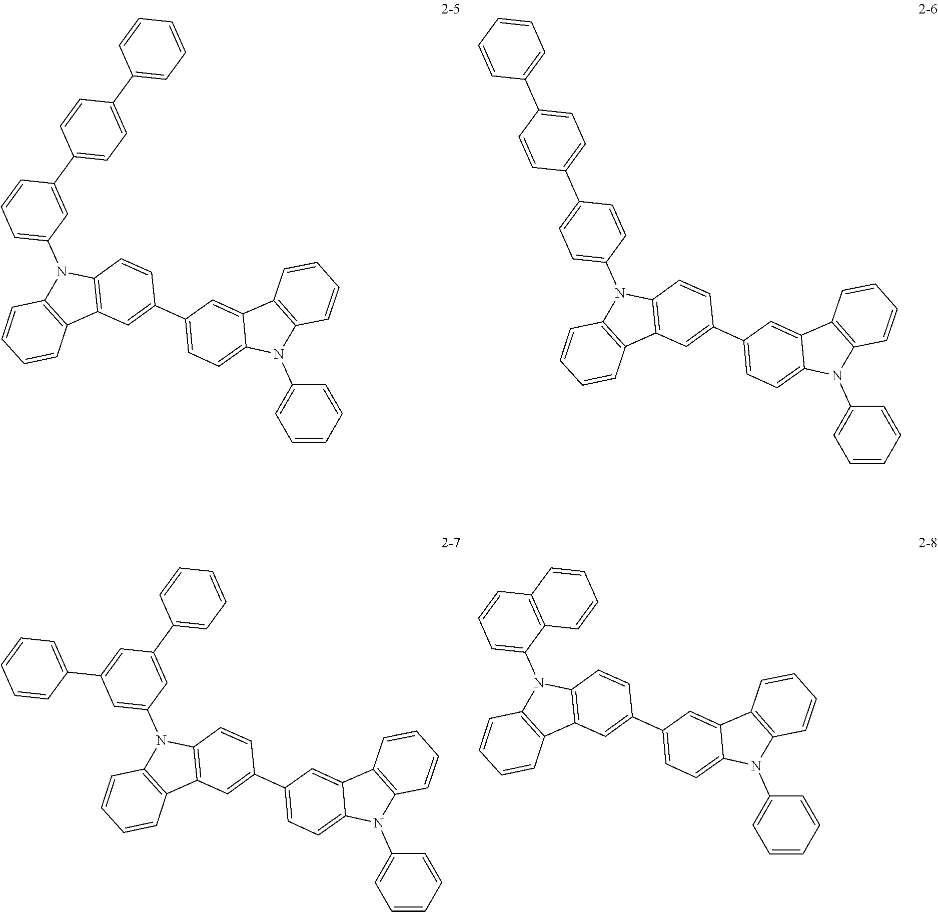

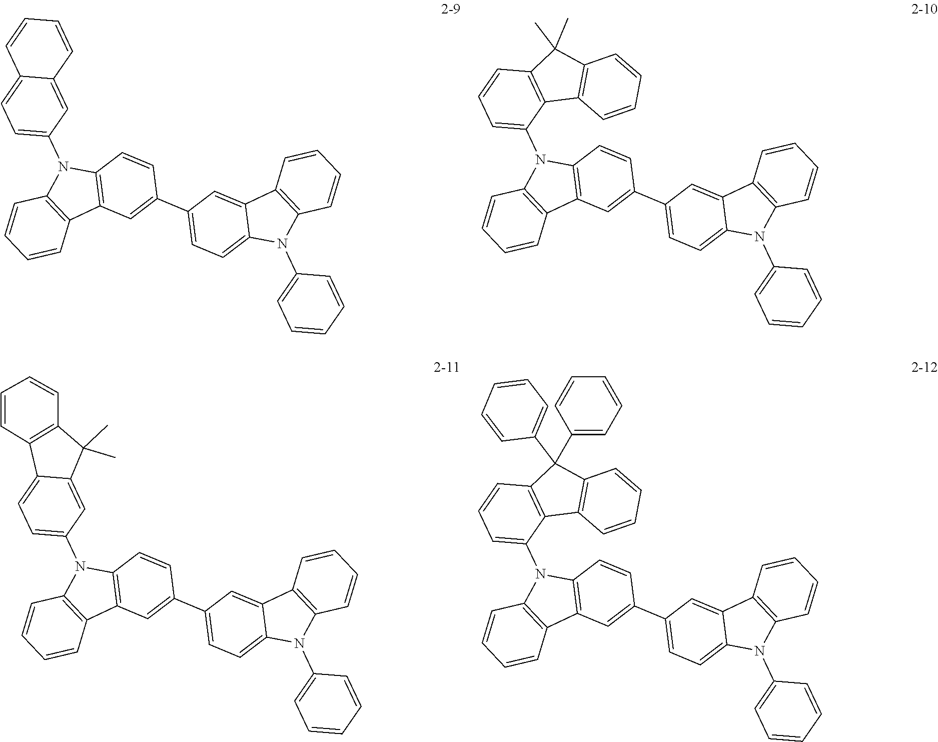 [SMILES: c1ccc(-n2c3ccccc3c3cc(-c4ccc5c(c4)c4ccccc4n5-c4ccc5ccccc5c4)ccc32)cc1 ; CC1(C)c2ccccc2-c2c(-n3c4ccccc4c4cc(-c5ccc6c(c5)c5ccccc5n6-c5ccccc5)ccc43)cccc21 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4cccc5c4-c4ccccc4C5(c4ccccc4)c4ccccc4)ccc32)cc1]

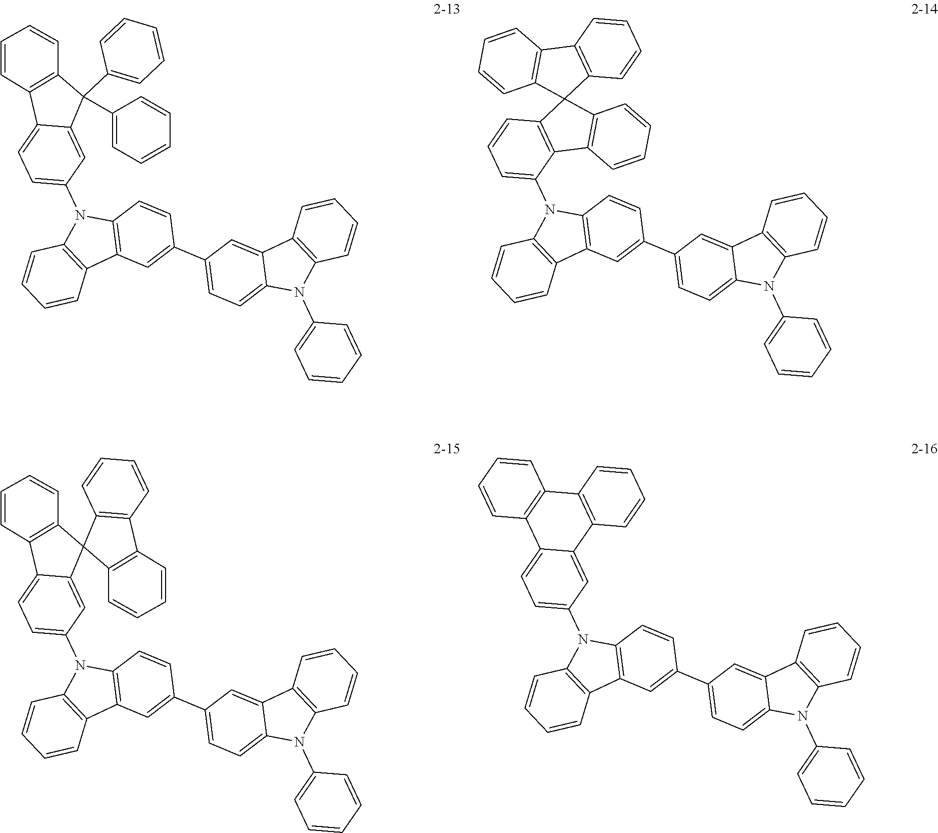 [SMILES: c1ccc(-n2c3ccccc3c3cc(-c4ccc5c(c4)c4ccccc4n5-c4ccc5c(c4)C(c4ccccc4)(c4ccccc4)c4ccccc4-5)ccc32)cc1 ; c1ccc(-n2c3ccccc3c3cc(-c4ccc5c(c4)c4ccccc4n5-c4cccc5c4-c4ccccc4C54c5ccccc5-c5ccccc54)ccc32)cc1 ; c1ccc(-n2c3ccccc3c3cc(-c4ccc5c(c4)c4ccccc4n5-c4ccc5c(c4)C4(c6ccccc6-c6ccccc64)c4ccccc4-5)ccc32)cc1 ; c1ccc(-n2c3ccccc3c3cc(-c4ccc5c(c4)c4ccccc4n5-c4ccc5c6ccccc6c6ccccc6c5c4)ccc32)cc1]

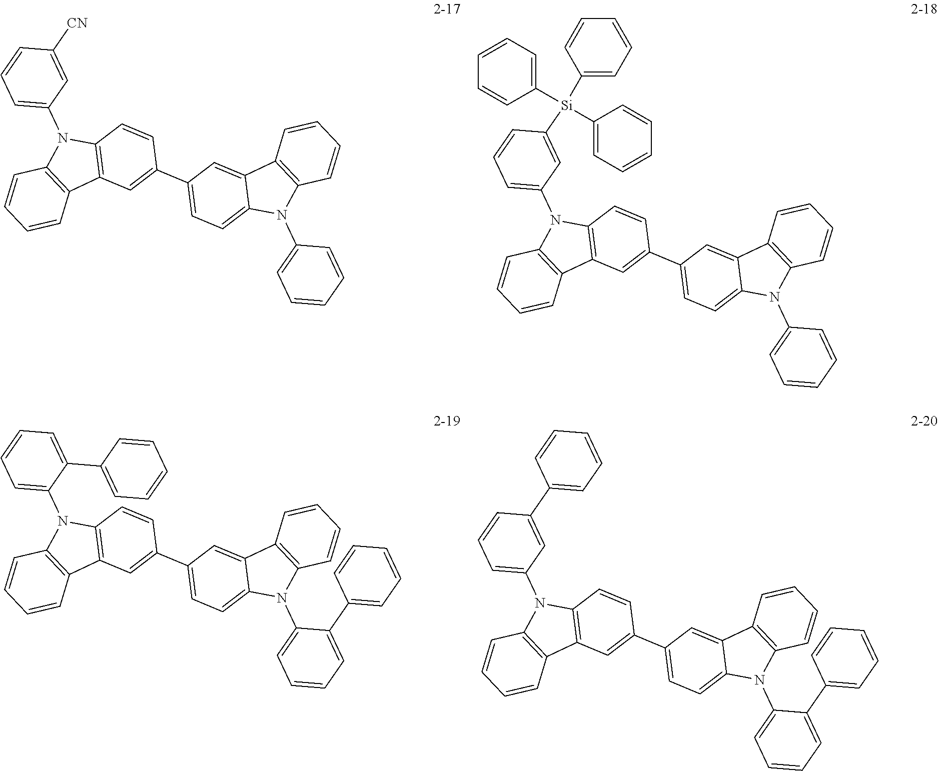 [SMILES: N#Cc1cccc(-n2c3ccccc3c3cc(-c4ccc5c(c4)c4ccccc4n5-c4ccccc4)ccc32)c1 ; c1ccc(-n2c3ccccc3c3cc(-c4ccc5c(c4)c4ccccc4n5-c4cccc([Si](c5ccccc5)(c5ccccc5)c5ccccc5)c4)ccc32)cc1 ; c1ccc(-c2ccccc2-n2c3ccccc3c3cc(-c4ccc5c(c4)c4ccccc4n5-c4ccccc4-c4ccccc4)ccc32)cc1 ; c1ccc(-c2cccc(-n3c4ccccc4c4cc(-c5ccc6c(c5)c5ccccc5n6-c5ccccc5-c5ccccc5)ccc43)c2)cc1]

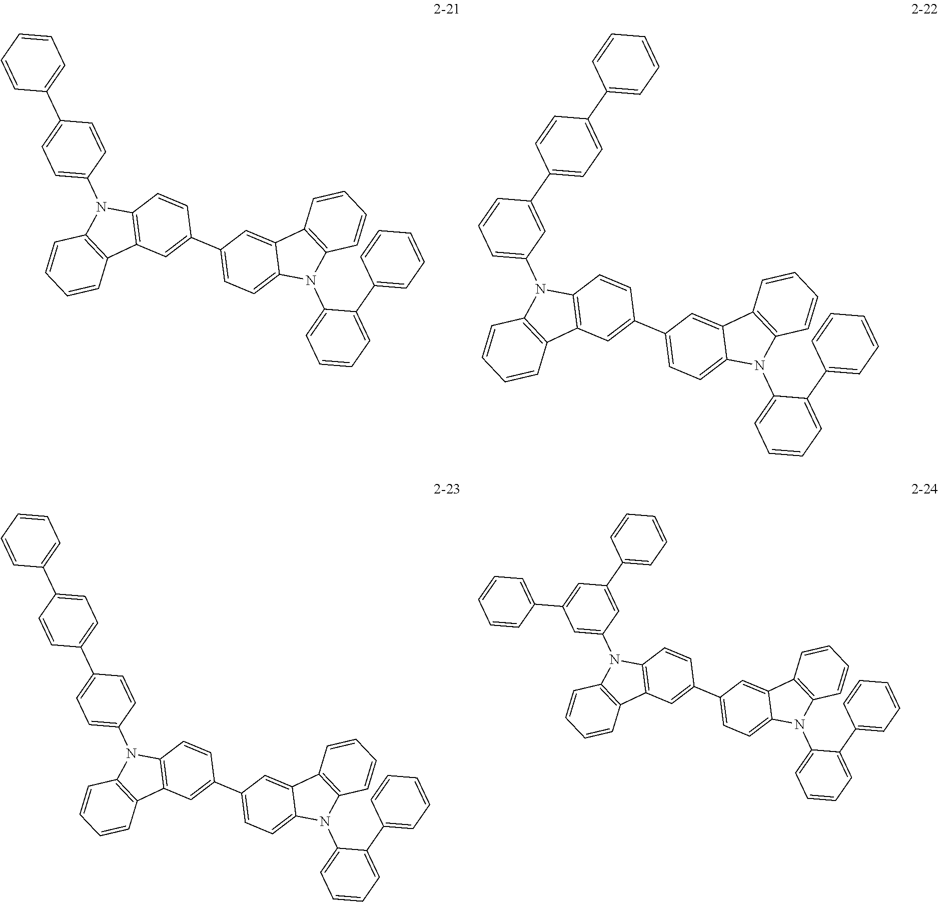 [SMILES: c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2ccc(-c3cccc(-n4c5ccccc5c5cc(-c6ccc7c(c6)c6ccccc6n7-c6ccccc6-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6-c6ccccc6)ccc54)cc3)cc2)cc1 ; c1ccc(-c2cc(-c3ccccc3)cc(-n3c4ccccc4c4cc(-c5ccc6c(c5)c5ccccc5n6-c5ccccc5-c5ccccc5)ccc43)c2)cc1]

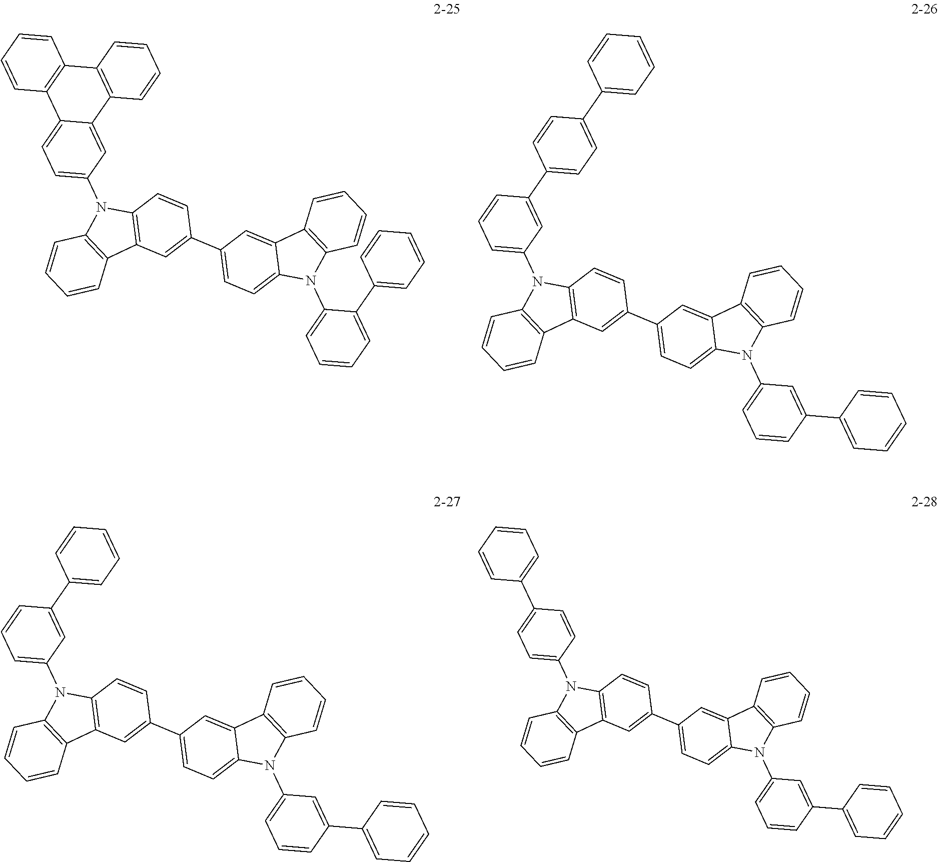 [SMILES: c1ccc(-c2ccccc2-n2c3ccccc3c3cc(-c4ccc5c(c4)c4ccccc4n5-c4ccc5c6ccccc6c6ccccc6c5c4)ccc32)cc1 ; c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2ccc(-n3c4ccccc4c4cc(-c5ccc6c(c5)c5ccccc5n6-c5cccc(-c6ccccc6)c5)ccc43)cc2)cc1]

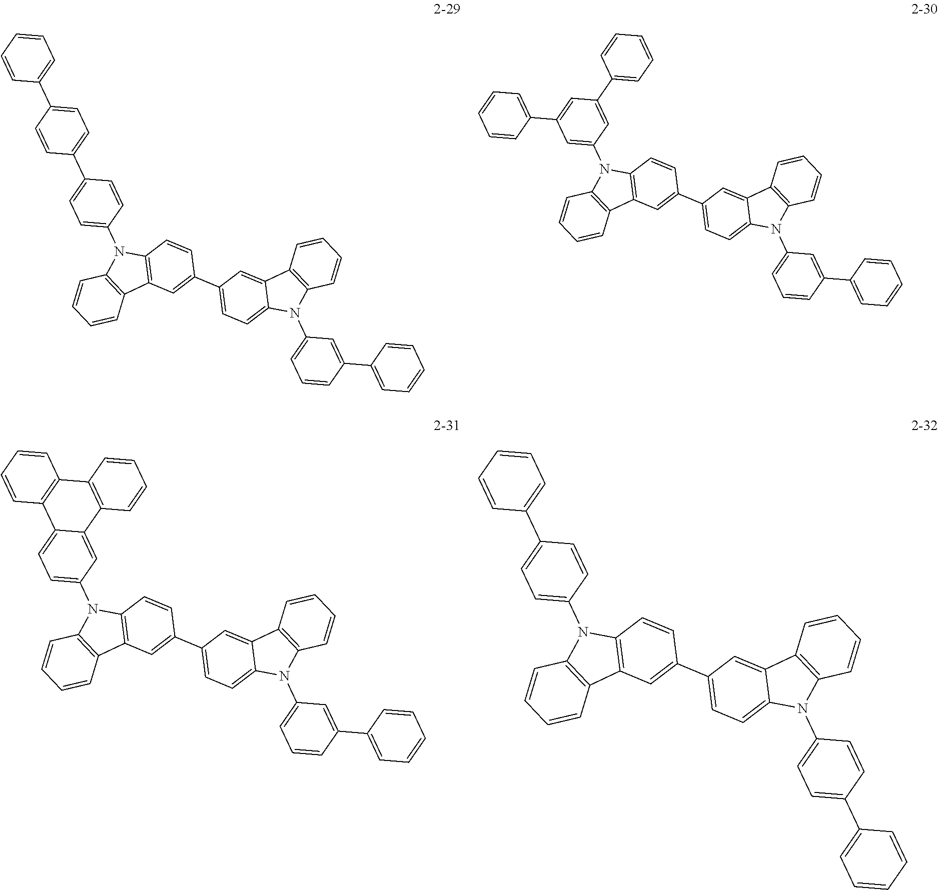 [SMILES: c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cccc(-n3c4ccccc4c4cc(-c5ccc6c(c5)c5ccccc5n6-c5ccc6c7ccccc7c7ccccc7c6c5)ccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

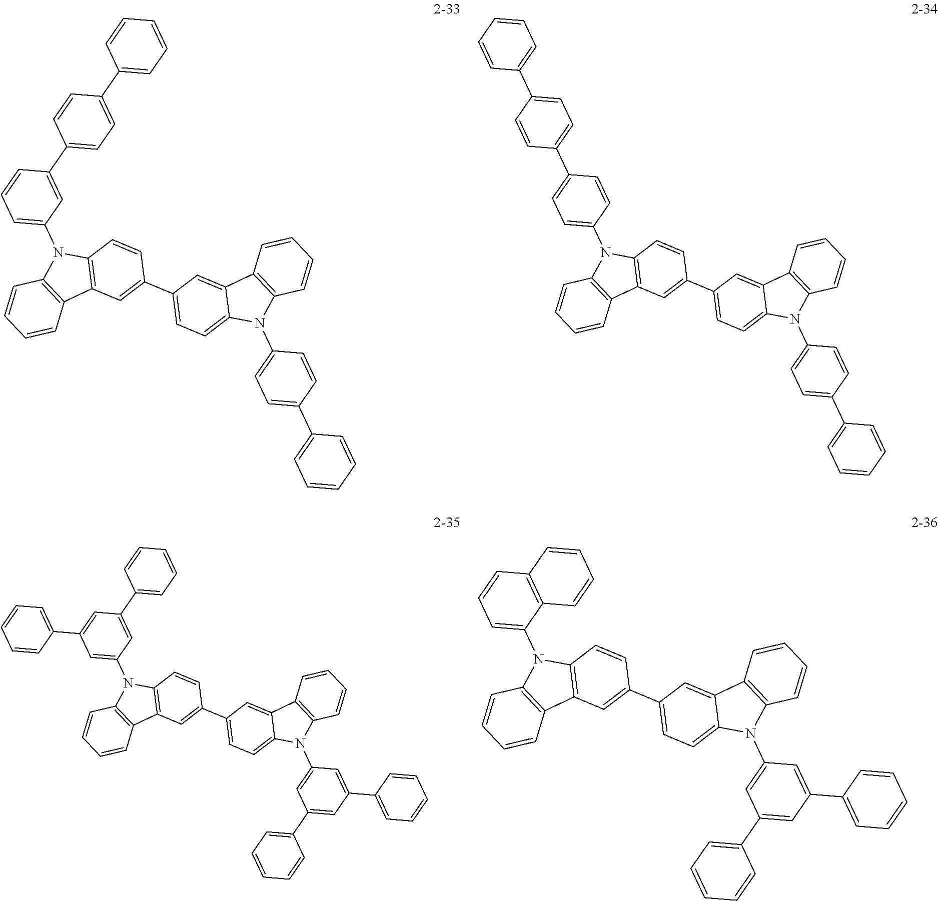 [SMILES: c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1 ; c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1 ; c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cc(-c3ccccc3)cc(-n3c4ccccc4c4cc(-c5ccc6c(c5)c5ccccc5n6-c5cccc6ccccc56)ccc43)c2)cc1]

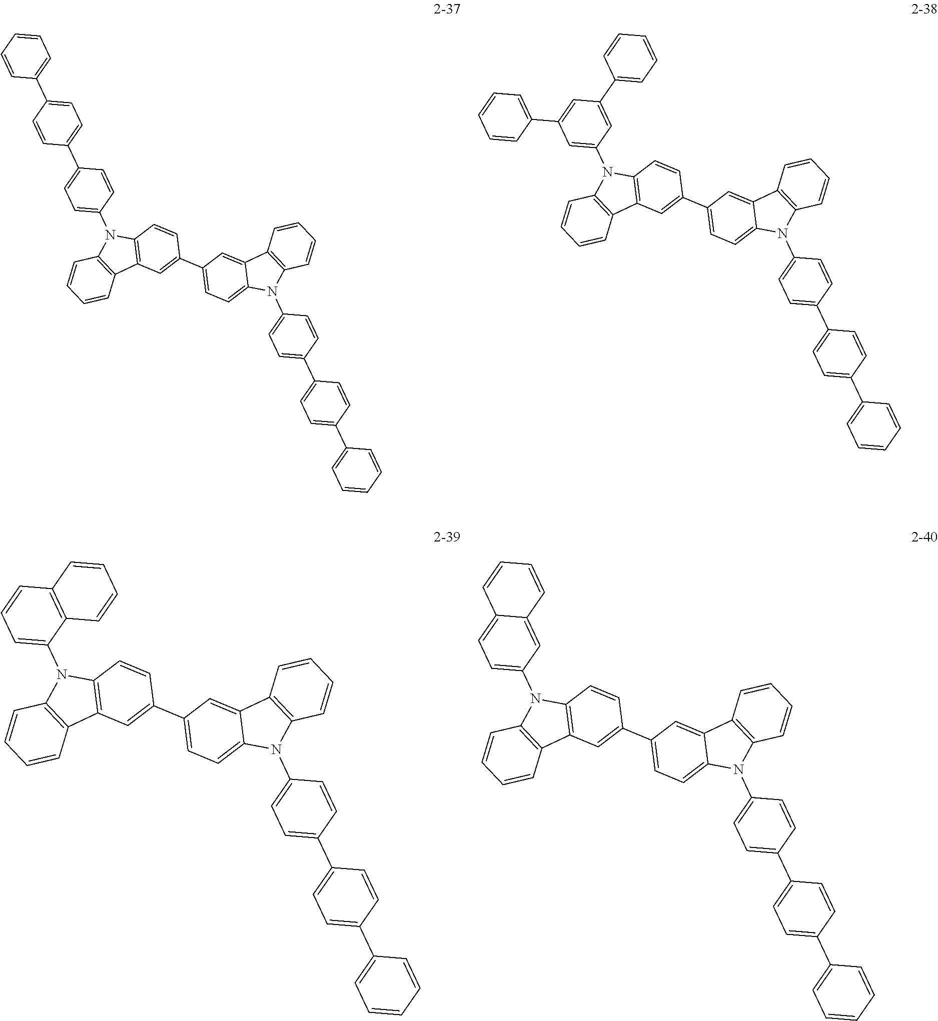 [SMILES: c1ccc(-c2ccc(-c3ccc(-n4c5ccccc5c5cc(-c6ccc7c(c6)c6ccccc6n7-c6ccc(-c7ccc(-c8ccccc8)cc7)cc6)ccc54)cc3)cc2)cc1 ; c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1 ; c1ccc(-c2ccc(-c3ccc(-n4c5ccccc5c5cc(-c6ccc7c(c6)c6ccccc6n7-c6cccc7ccccc67)ccc54)cc3)cc2)cc1 ; c1ccc(-c2ccc(-c3ccc(-n4c5ccccc5c5cc(-c6ccc7c(c6)c6ccccc6n7-c6ccc7ccccc7c6)ccc54)cc3)cc2)cc1]

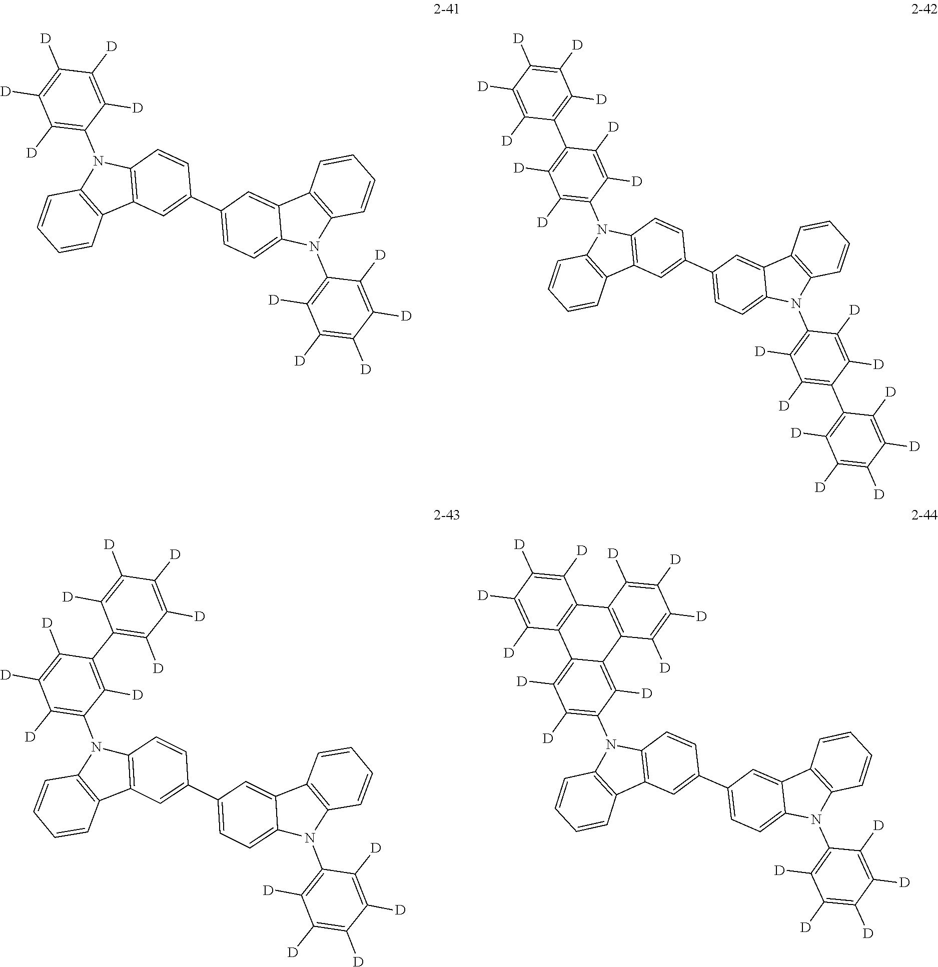

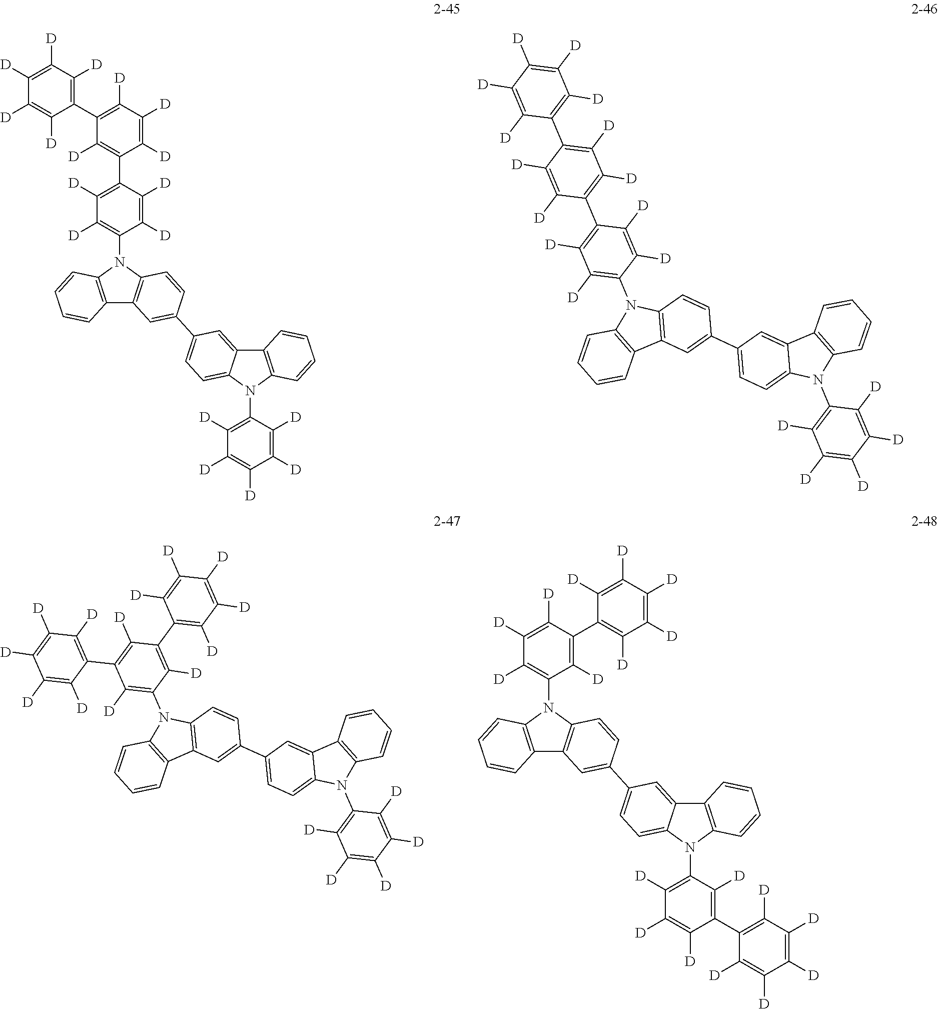

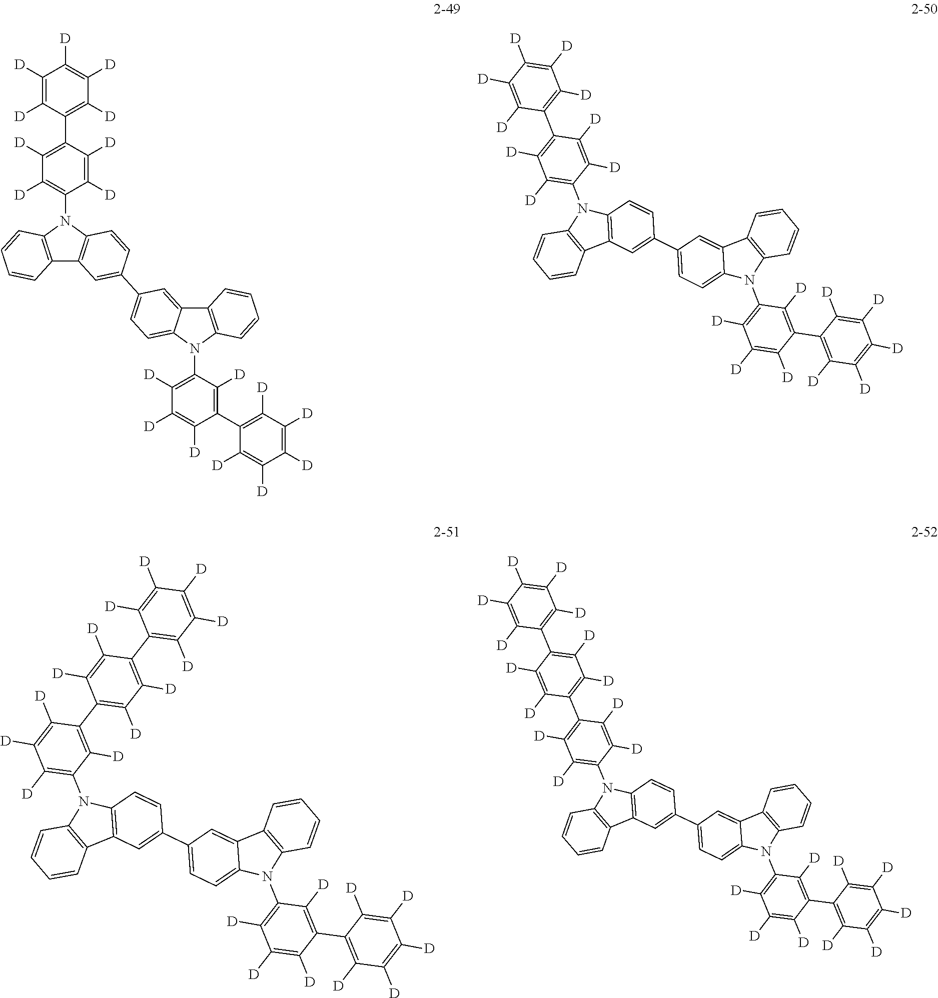

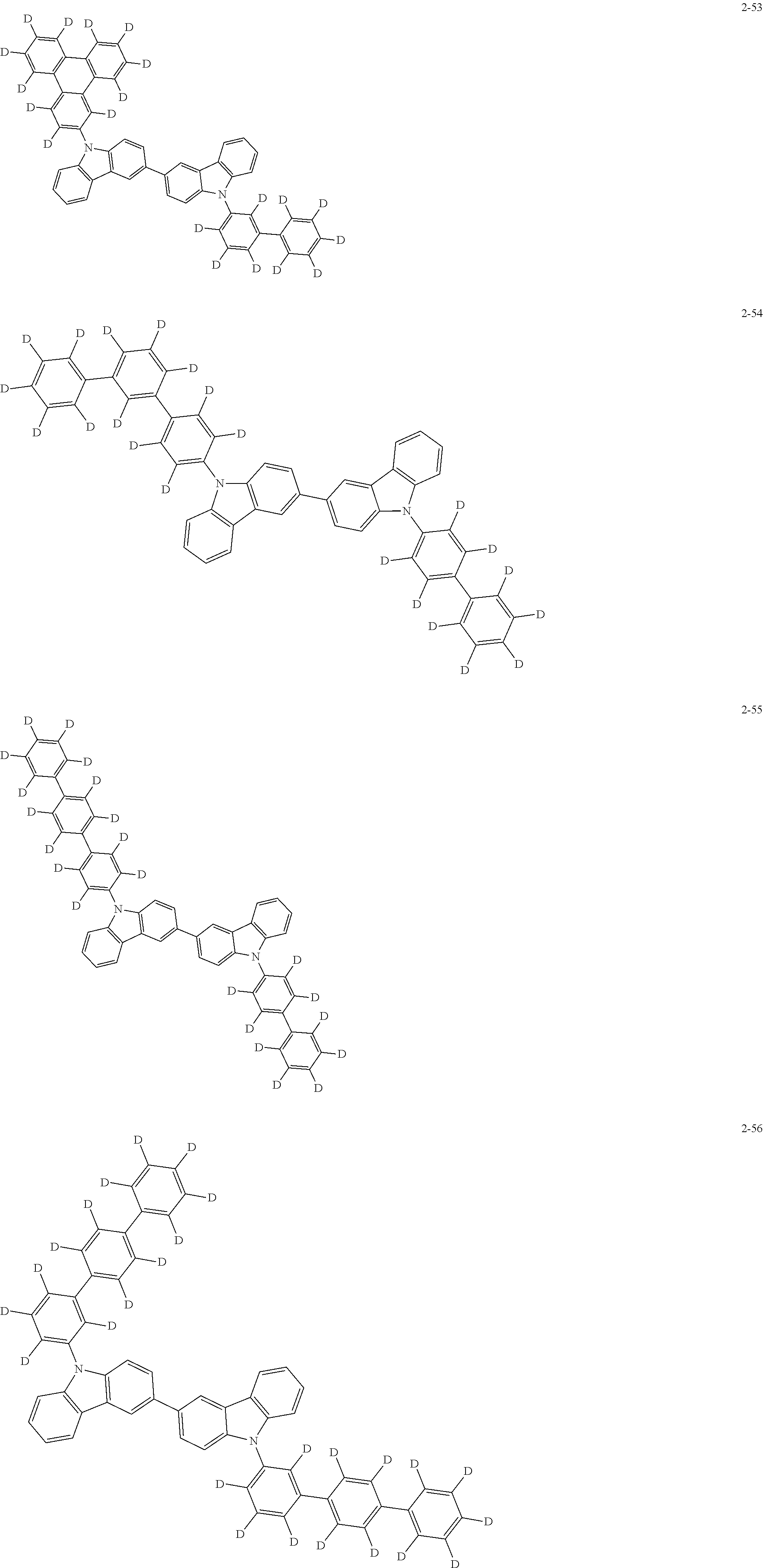

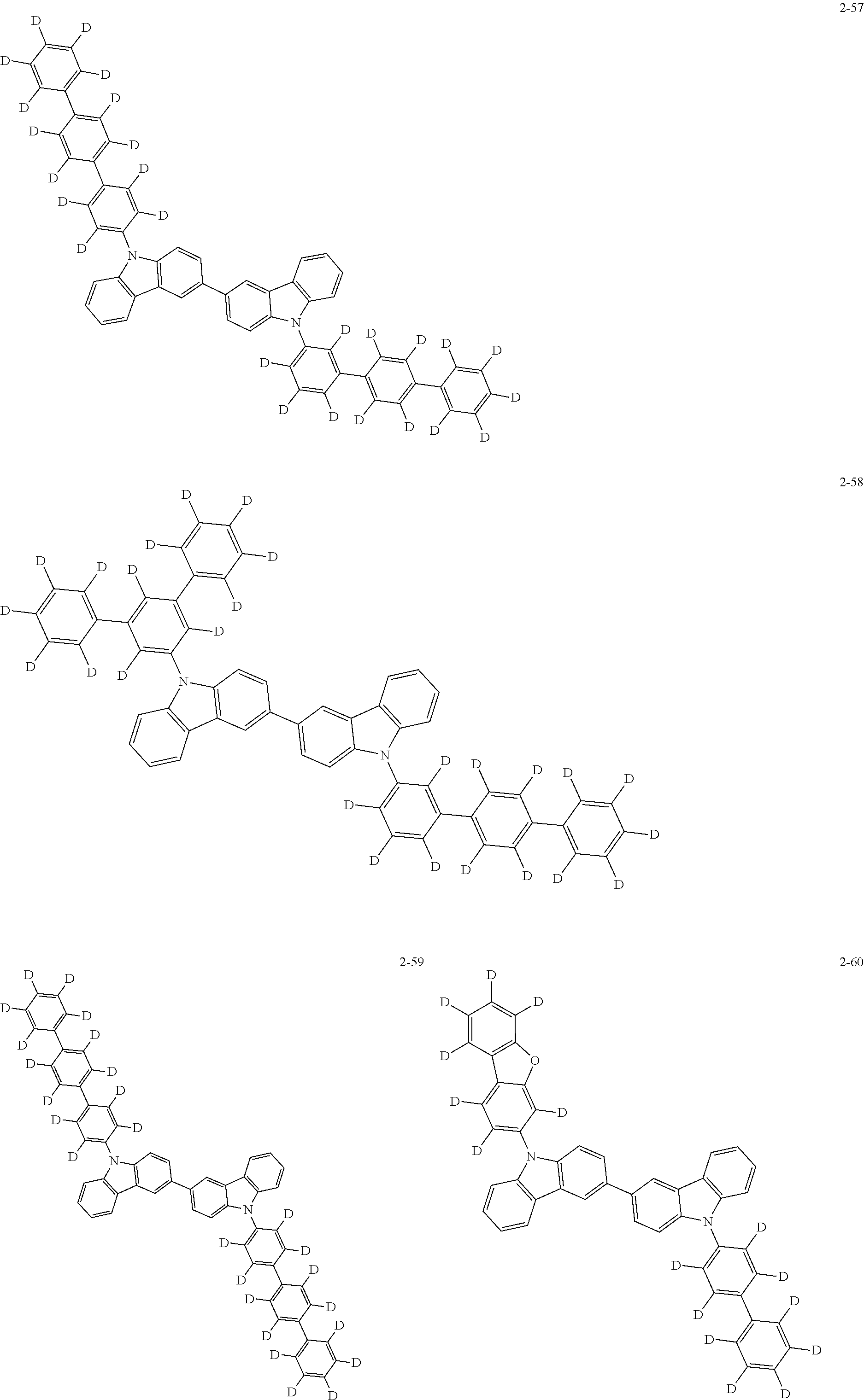

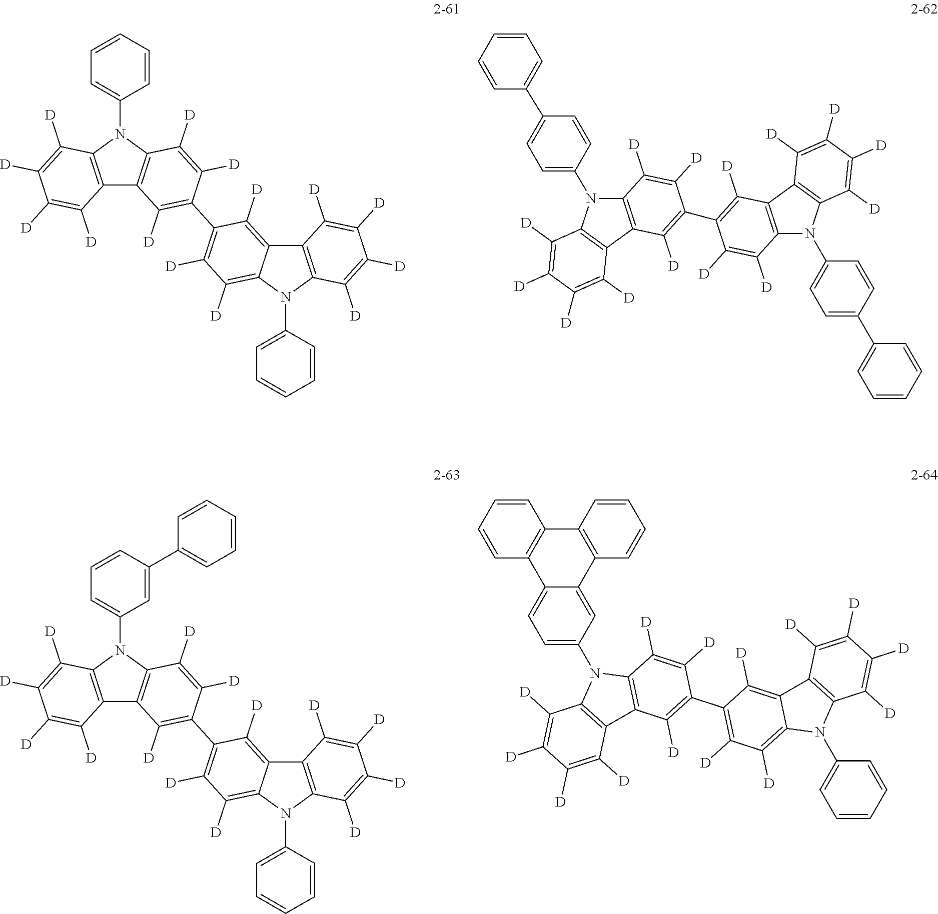

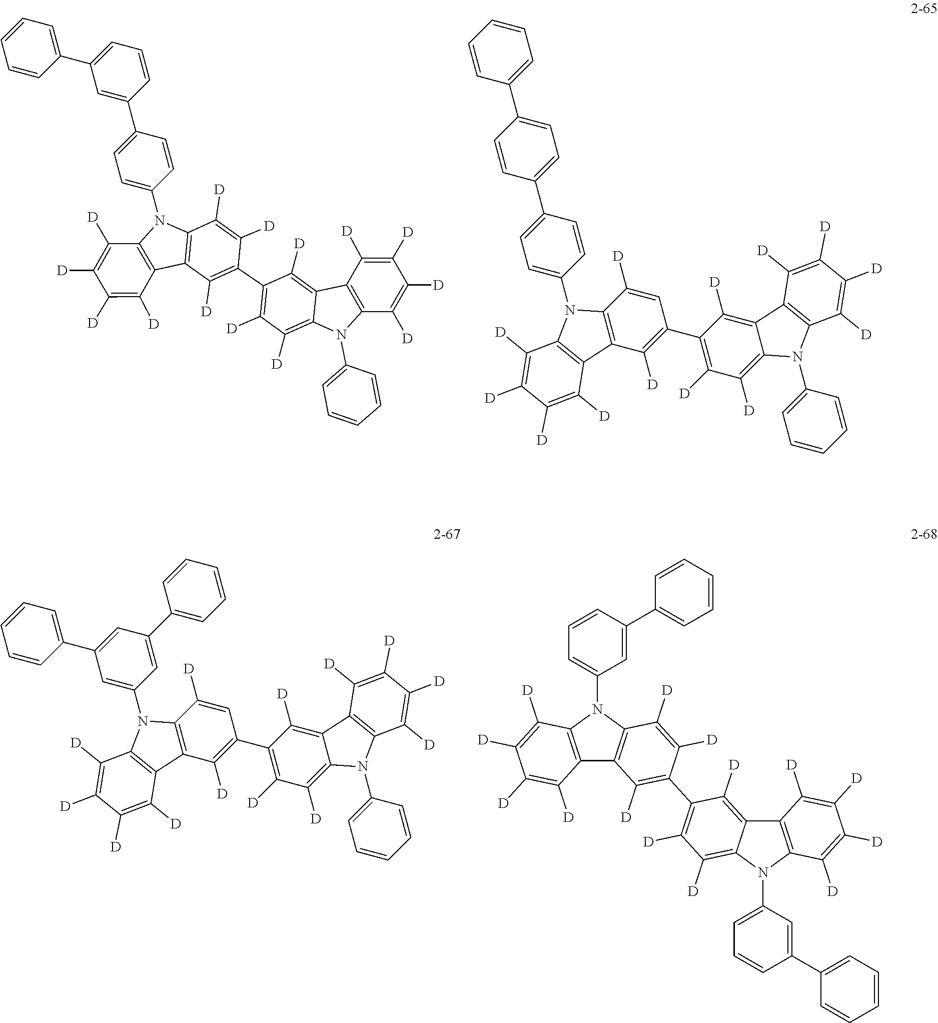

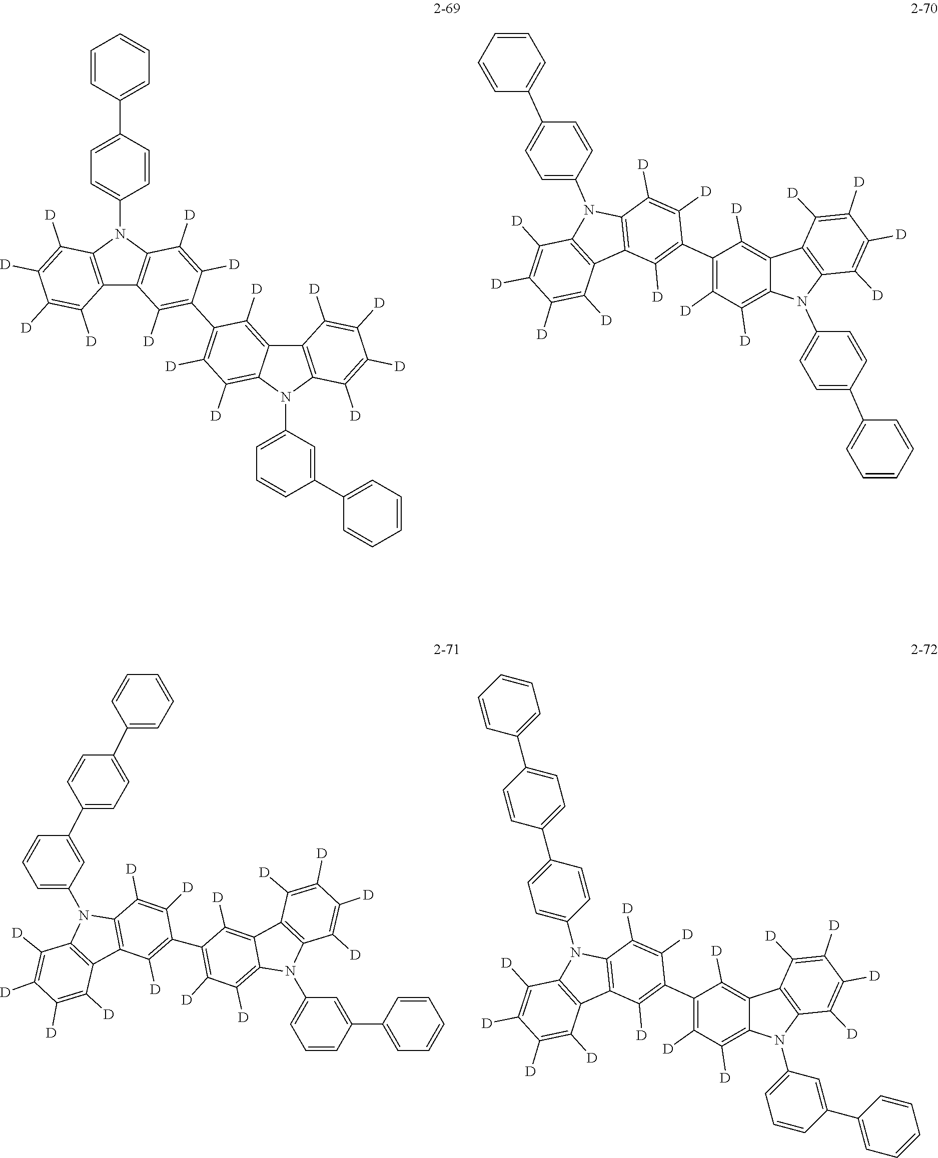

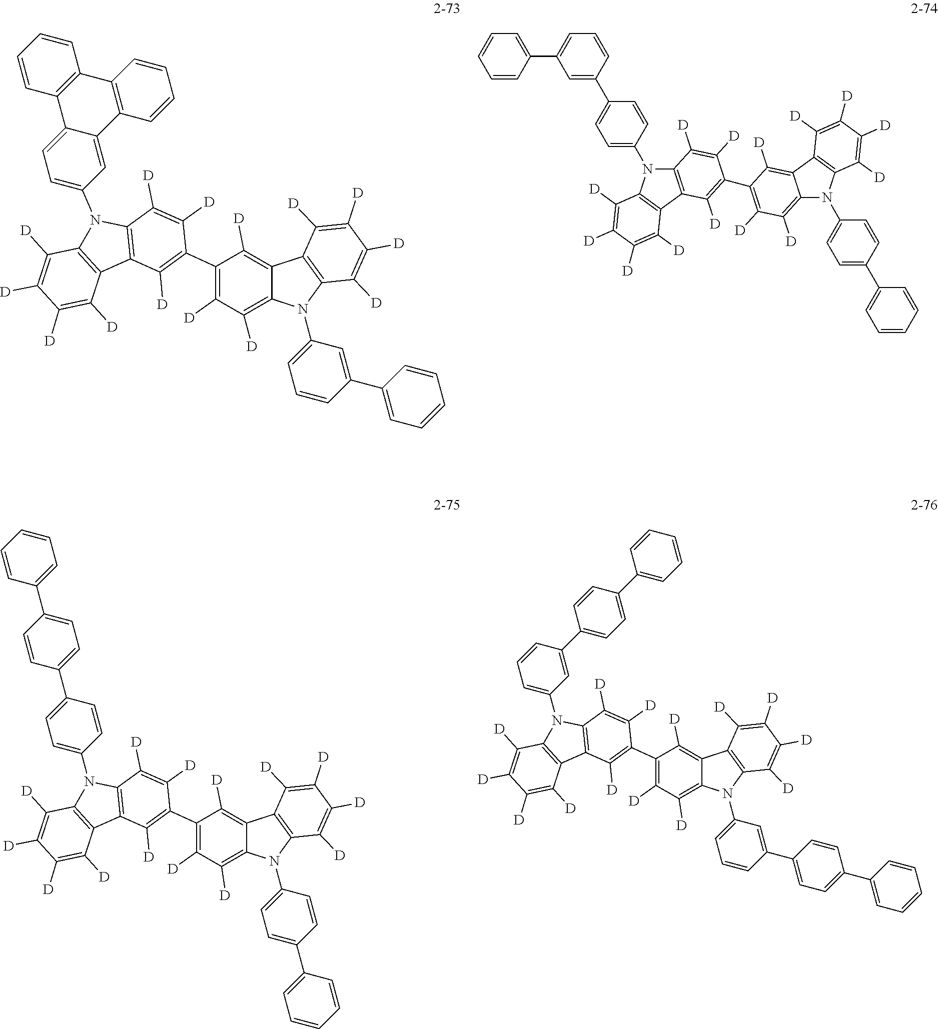

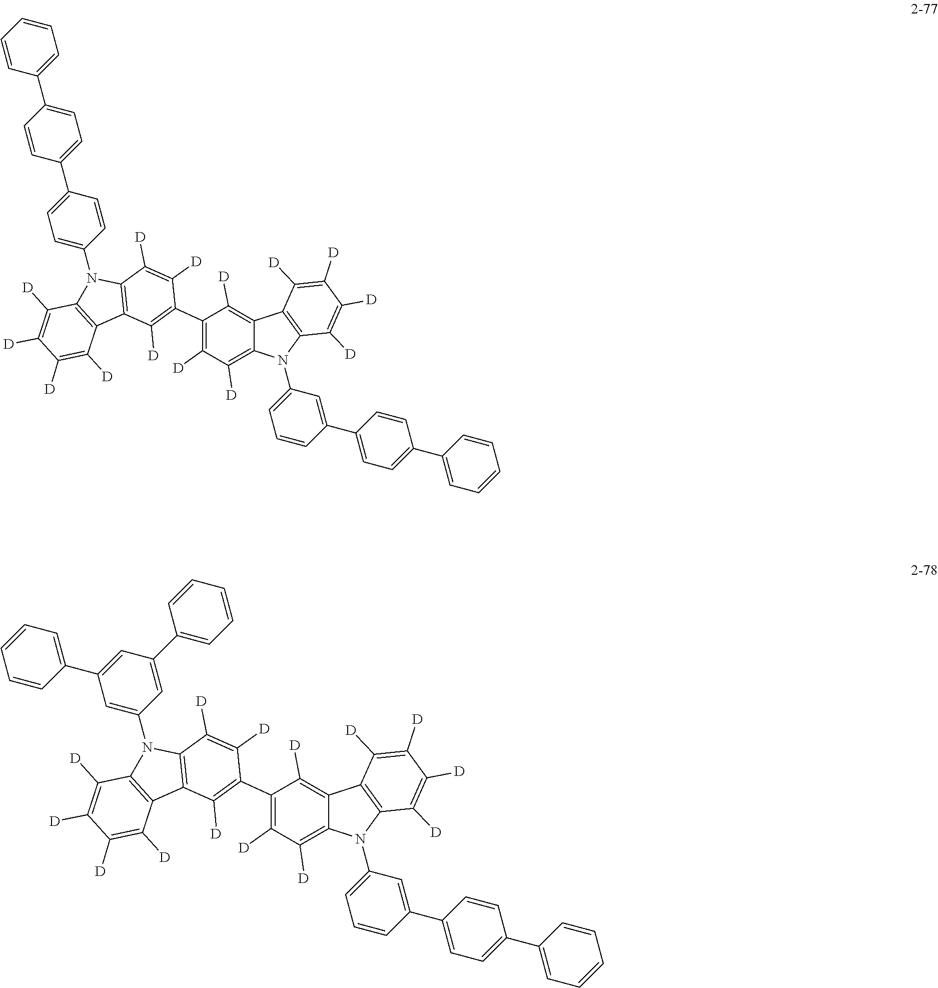 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccc(-c5ccccc5)cc4)c3)c([2H])c([2H])c1n2-c1ccc(-c2ccc(-c3ccccc3)cc2)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cc(-c4ccccc4)cc(-c4ccccc4)c3)c([2H])c([2H])c1n2-c1cccc(-c2ccc(-c3ccccc3)cc2)c1]

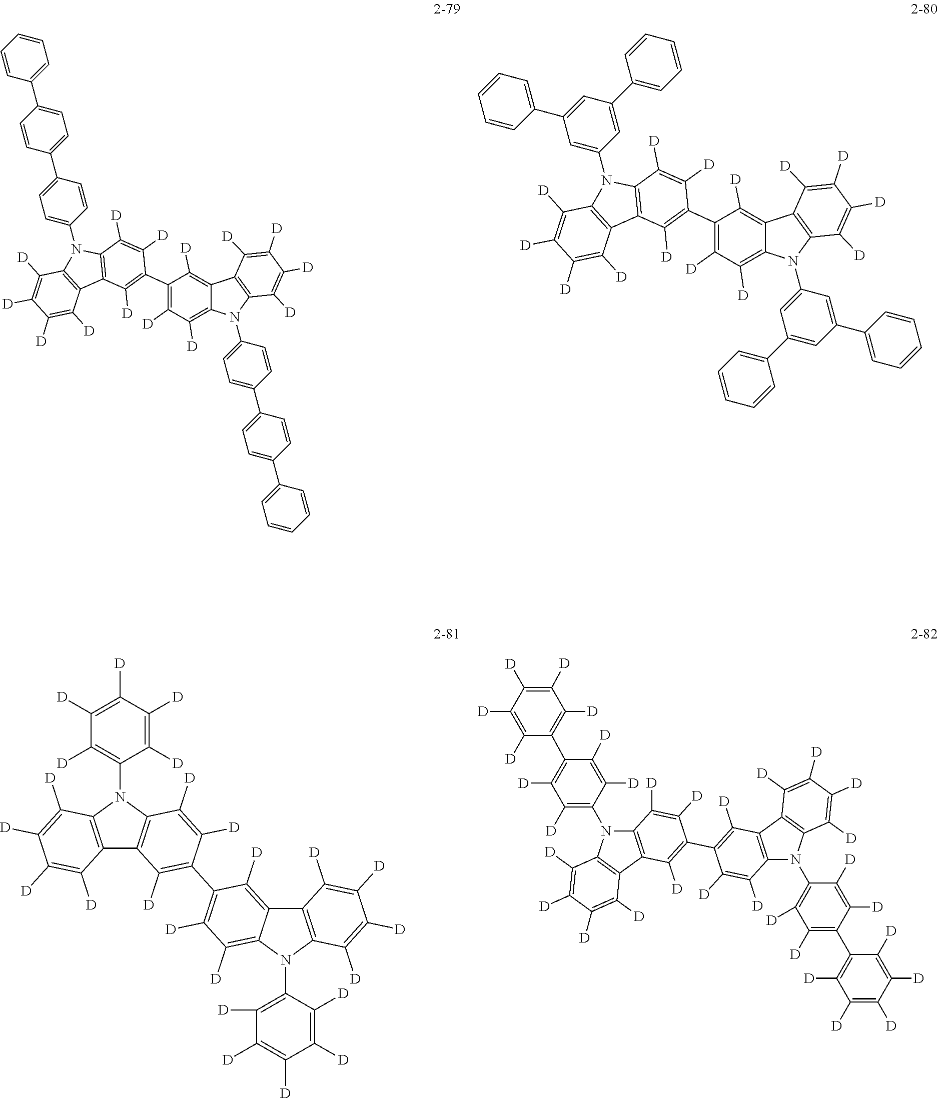

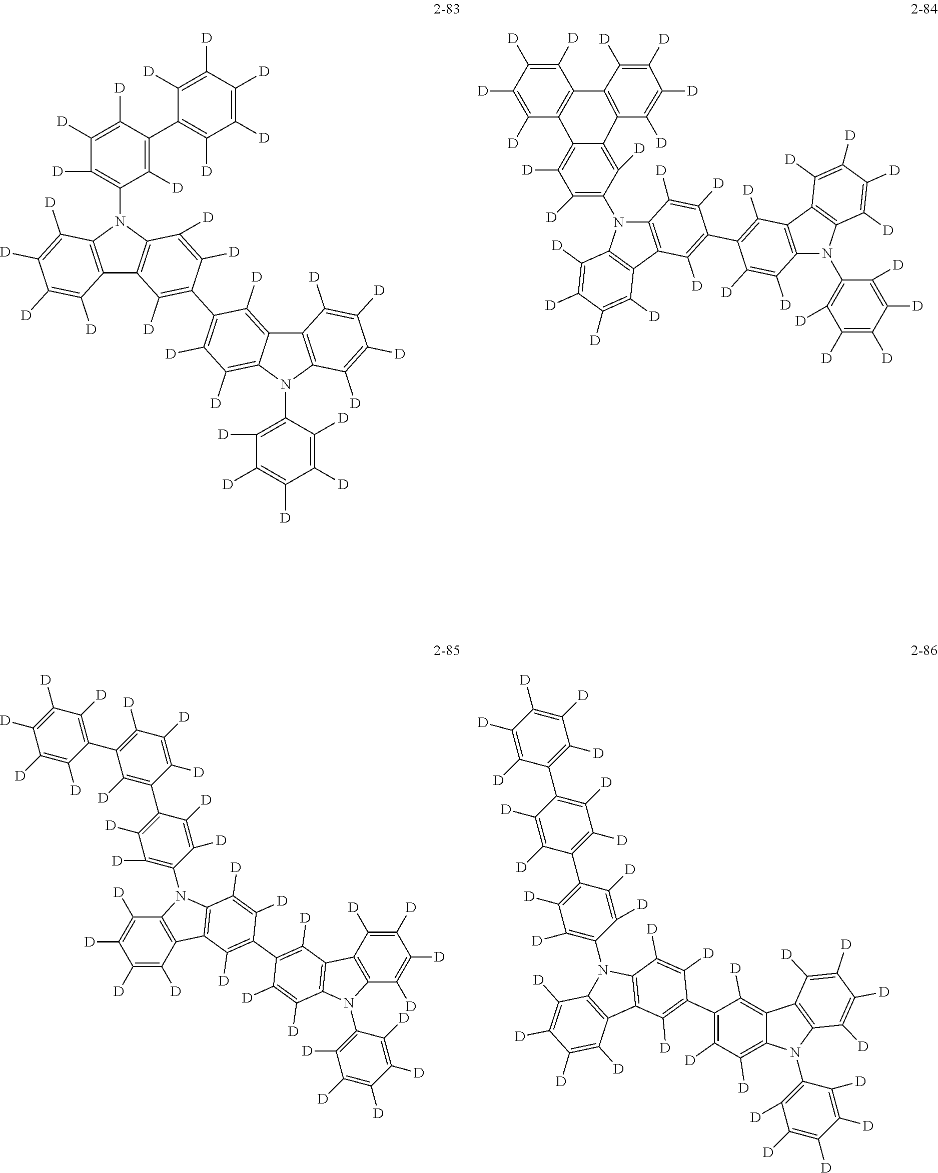

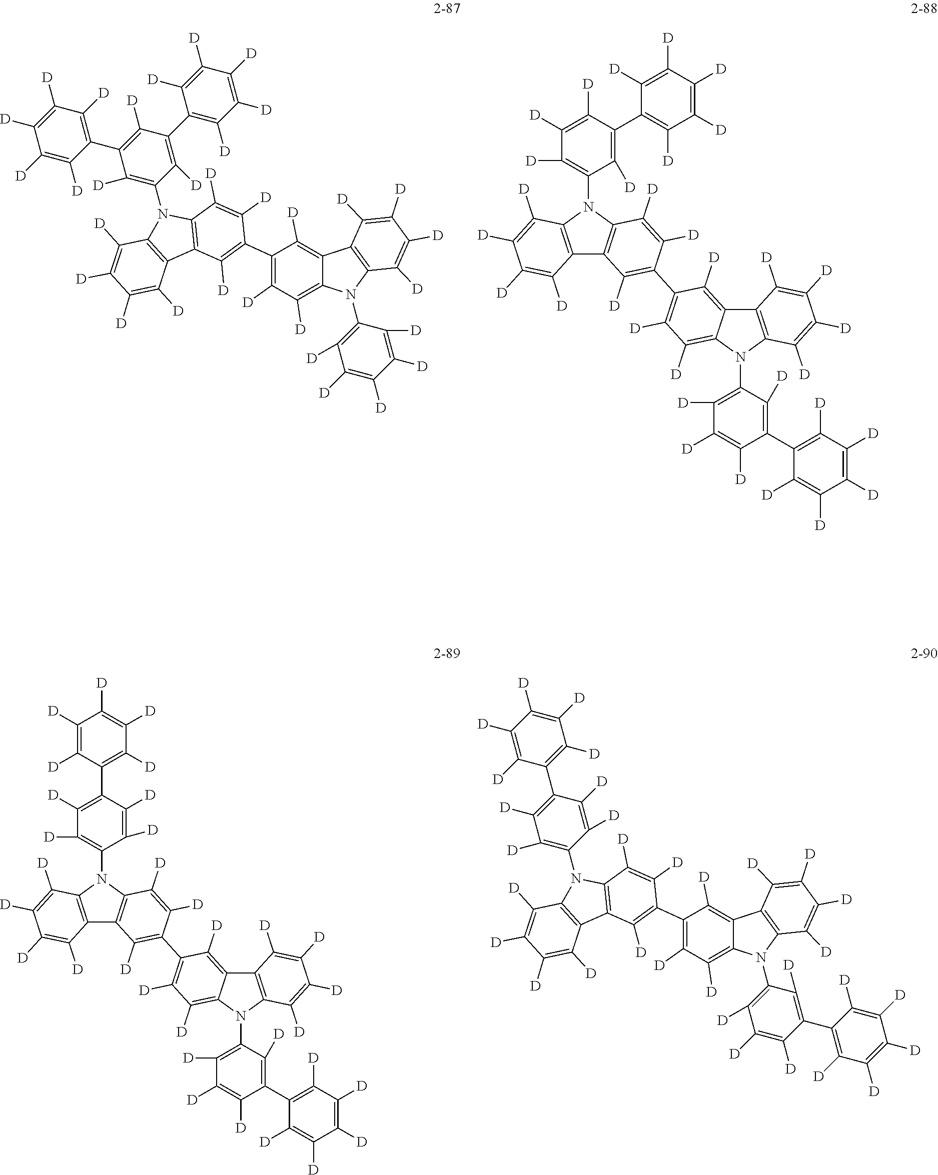

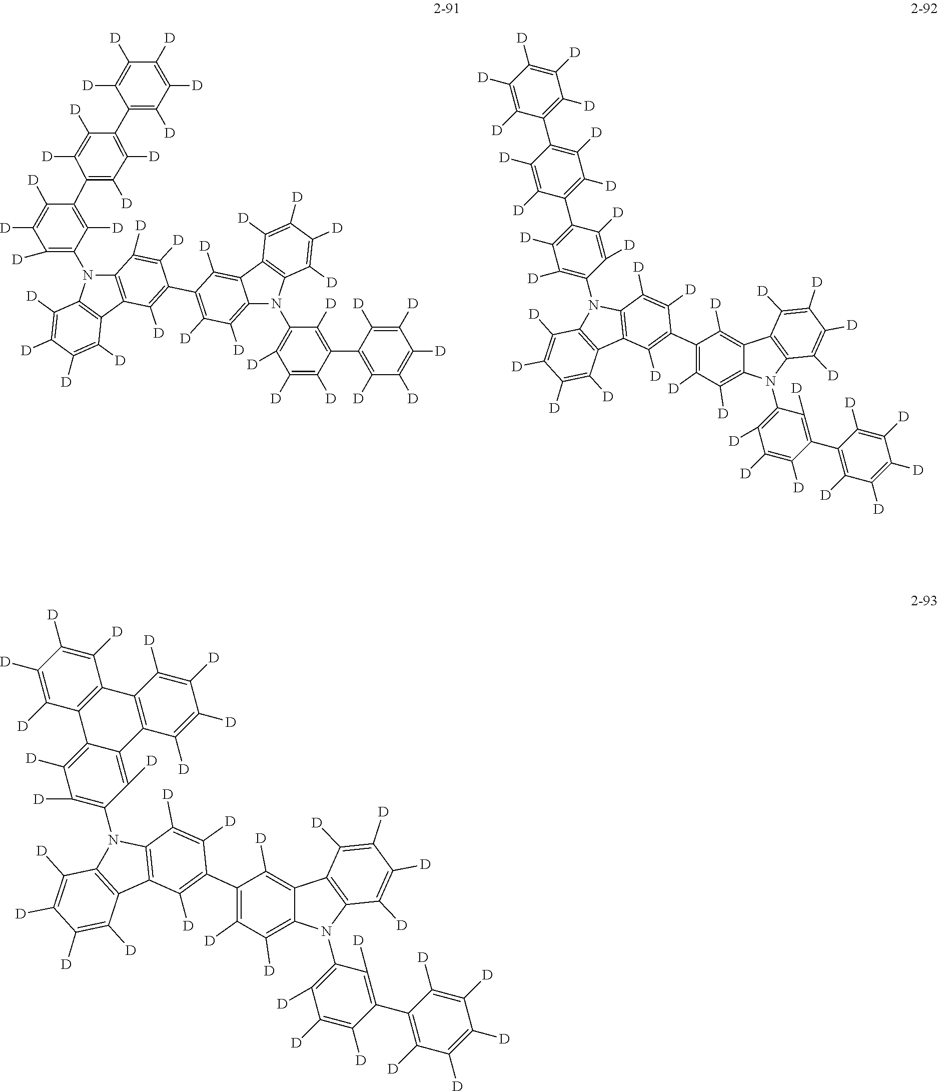

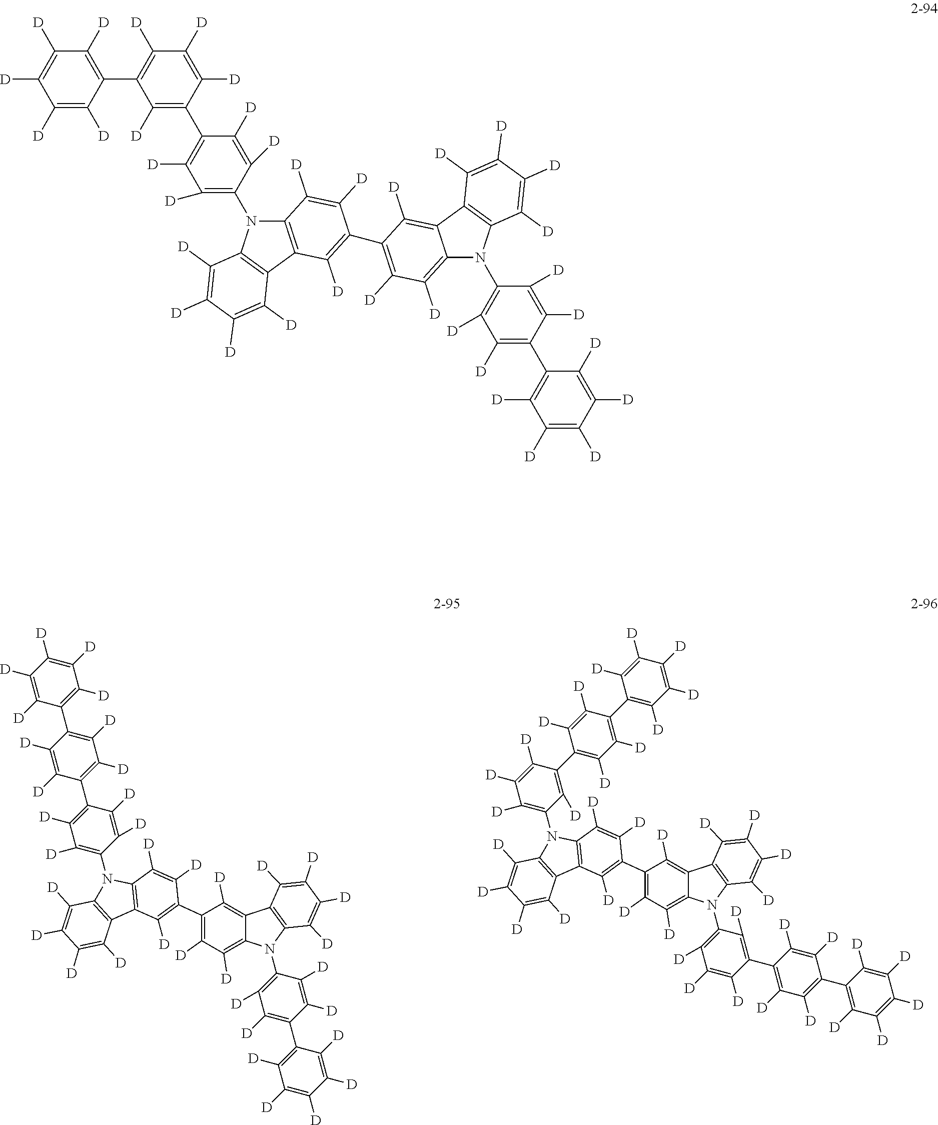

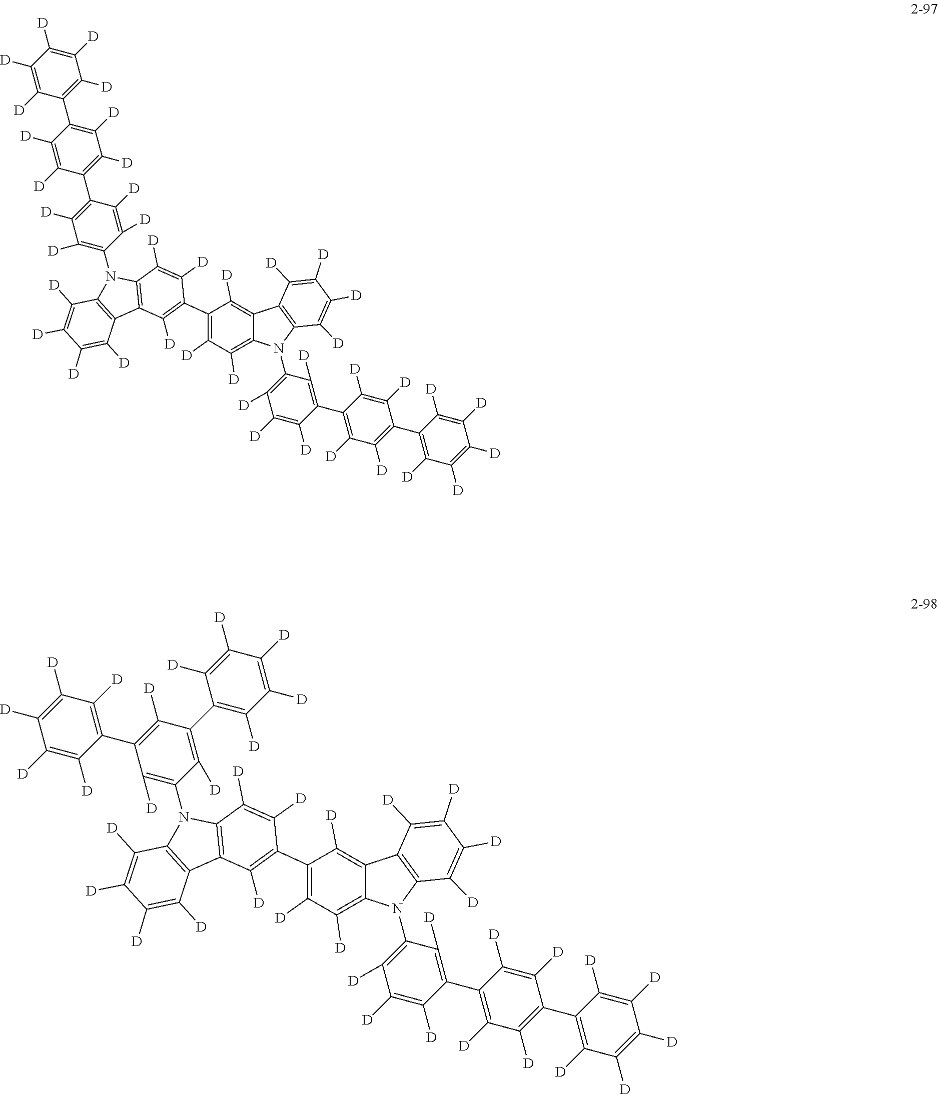

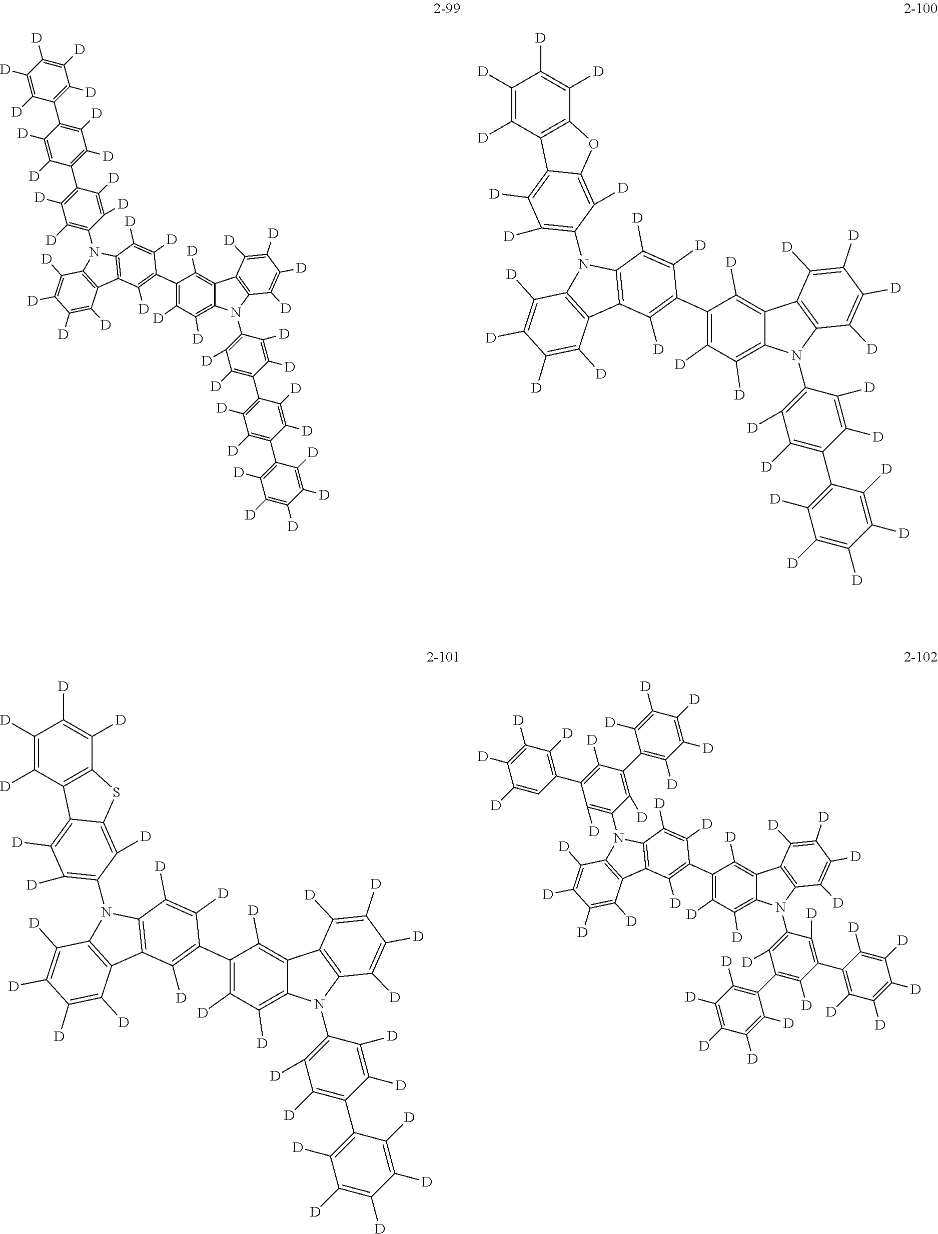

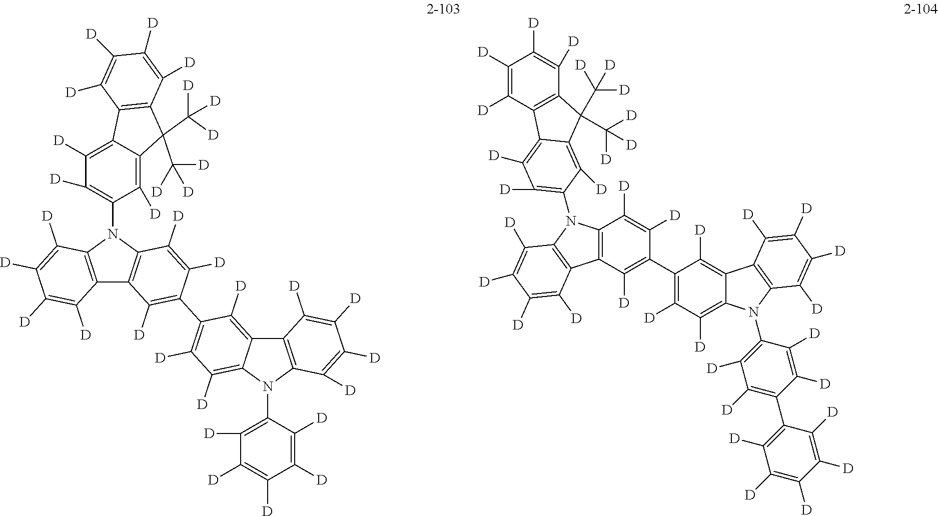

One embodiment of the present application provides a heterocyclic compound, wherein Chemical Formula 3 is represented by any one of the following compounds. In addition, in one embodiment of the present application, the following compounds are just one example, and the present application is not limited thereto and may include other compounds included in Chemical Formula 3 including additional substituents.

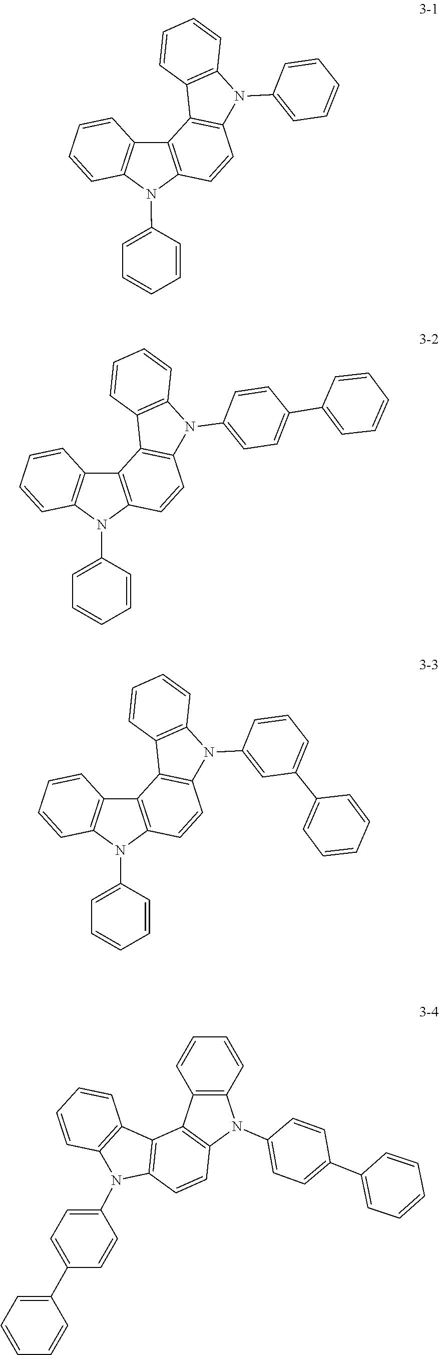 [SMILES: c1ccc(-n2c3ccccc3c3c4c5ccccc5n(-c5ccccc5)c4ccc32)cc1 ; c1ccc(-c2ccc(-n3c4ccccc4c4c5c6ccccc6n(-c6ccccc6)c5ccc43)cc2)cc1 ; c1ccc(-c2cccc(-n3c4ccccc4c4c5c6ccccc6n(-c6ccccc6)c5ccc43)c2)cc1 ; c1ccc(-c2ccc(-n3c4ccccc4c4c5c6ccccc6n(-c6ccc(-c7ccccc7)cc6)c5ccc43)cc2)cc1]

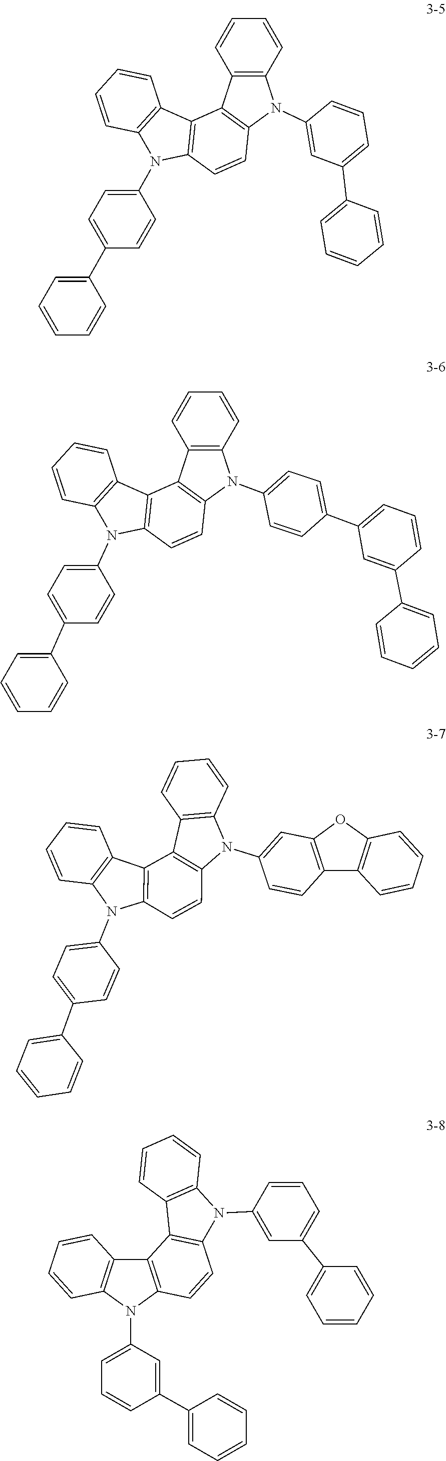 [SMILES: c1ccc(-c2ccc(-n3c4ccccc4c4c5c6ccccc6n(-c6cccc(-c7ccccc7)c6)c5ccc43)cc2)cc1 ; c1ccc(-c2ccc(-n3c4ccccc4c4c5c6ccccc6n(-c6ccc(-c7cccc(-c8ccccc8)c7)cc6)c5ccc43)cc2)cc1 ; c1ccc(-c2ccc(-n3c4ccccc4c4c5c6ccccc6n(-c6ccc7c(c6)oc6ccccc67)c5ccc43)cc2)cc1 ; c1ccc(-c2cccc(-n3c4ccccc4c4c5c6ccccc6n(-c6cccc(-c7ccccc7)c6)c5ccc43)c2)cc1]

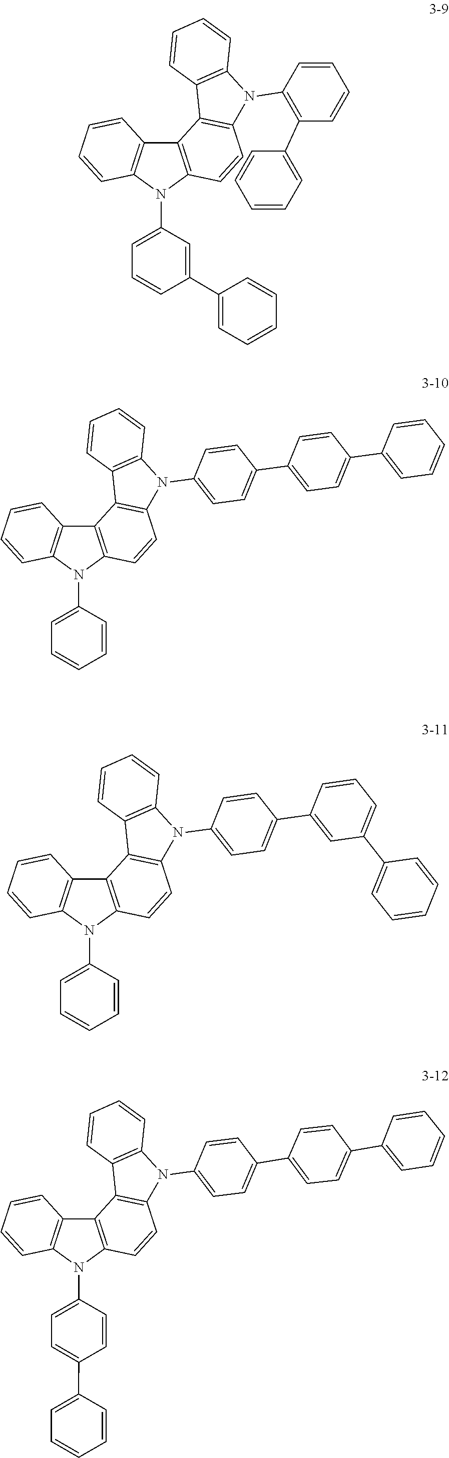 [SMILES: c1ccc(-c2cccc(-n3c4ccccc4c4c5c6ccccc6n(-c6ccccc6-c6ccccc6)c5ccc43)c2)cc1 ; c1ccc(-c2ccc(-c3ccc(-n4c5ccccc5c5c6c7ccccc7n(-c7ccccc7)c6ccc54)cc3)cc2)cc1 ; c1ccc(-c2cccc(-c3ccc(-n4c5ccccc5c5c6c7ccccc7n(-c7ccccc7)c6ccc54)cc3)c2)cc1 ; c1ccc(-c2ccc(-c3ccc(-n4c5ccccc5c5c6c7ccccc7n(-c7ccc(-c8ccccc8)cc7)c6ccc54)cc3)cc2)cc1]

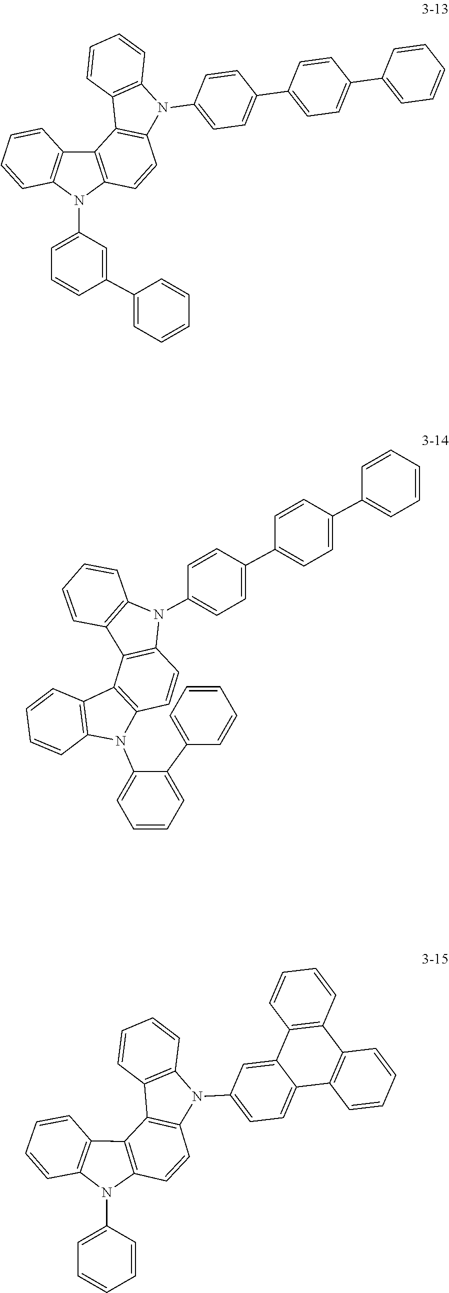 [SMILES: c1ccc(-c2ccc(-c3ccc(-n4c5ccccc5c5c6c7ccccc7n(-c7cccc(-c8ccccc8)c7)c6ccc54)cc3)cc2)cc1 ; c1ccc(-c2ccc(-c3ccc(-n4c5ccccc5c5c6c7ccccc7n(-c7ccccc7-c7ccccc7)c6ccc54)cc3)cc2)cc1 ; c1ccc(-n2c3ccccc3c3c4c5ccccc5n(-c5ccc6c7ccccc7c7ccccc7c6c5)c4ccc32)cc1]

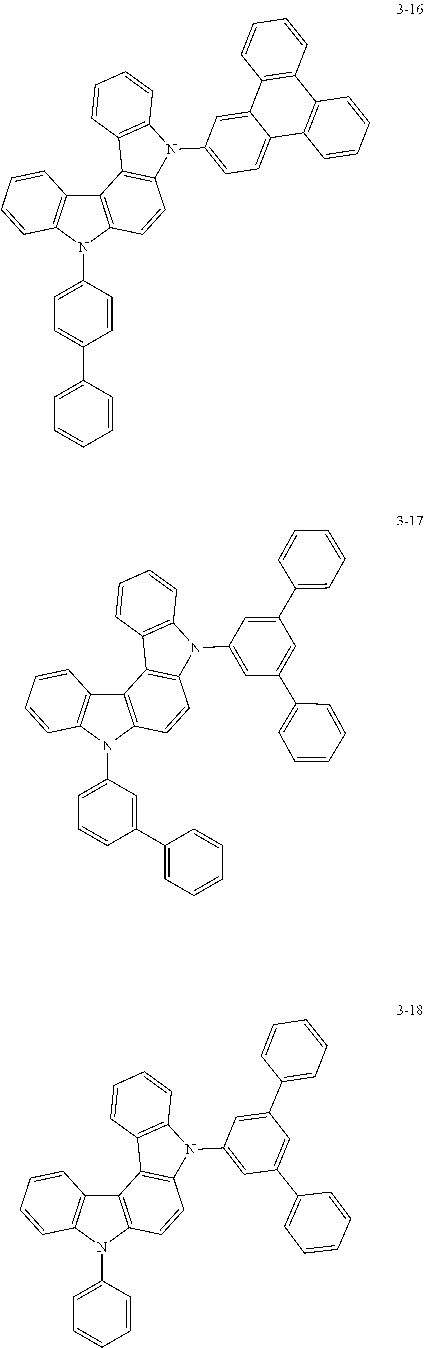 [SMILES: c1ccc(-c2ccc(-n3c4ccccc4c4c5c6ccccc6n(-c6ccc7c8ccccc8c8ccccc8c7c6)c5ccc43)cc2)cc1 ; c1ccc(-c2cccc(-n3c4ccccc4c4c5c6ccccc6n(-c6cc(-c7ccccc7)cc(-c7ccccc7)c6)c5ccc43)c2)cc1 ; c1ccc(-c2cc(-c3ccccc3)cc(-n3c4ccccc4c4c5c6ccccc6n(-c6ccccc6)c5ccc43)c2)cc1]

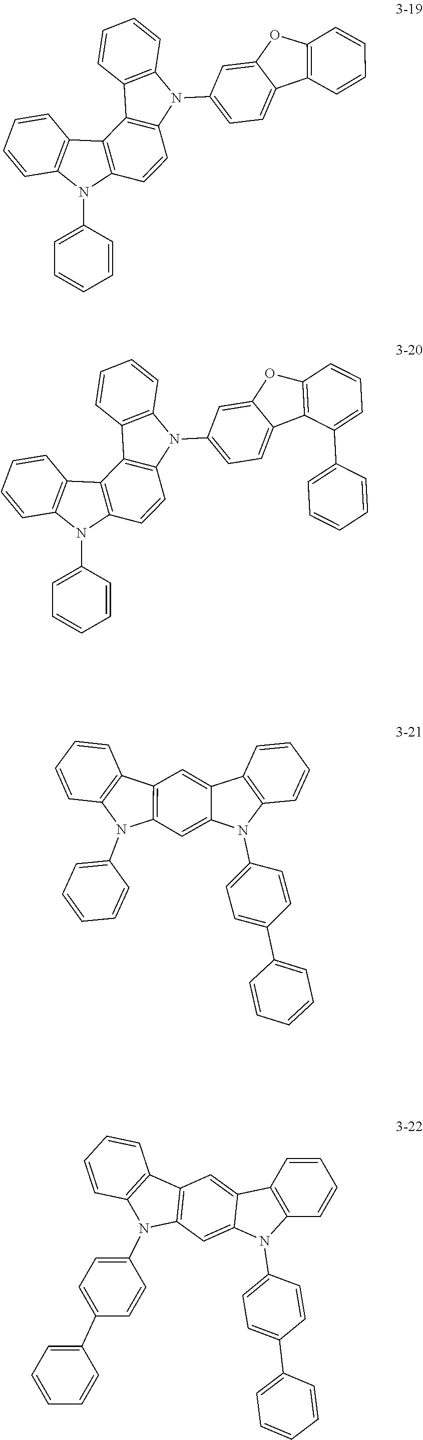 [SMILES: c1ccc(-n2c3ccccc3c3c4c5ccccc5n(-c5ccc6c(c5)oc5ccccc56)c4ccc32)cc1 ; c1ccc(-c2cccc3oc4cc(-n5c6ccccc6c6c7c8ccccc8n(-c8ccccc8)c7ccc65)ccc4c23)cc1 ; c1ccc(-c2ccc(-n3c4ccccc4c4cc5c6ccccc6n(-c6ccccc6)c5cc43)cc2)cc1 ; c1ccc(-c2ccc(-n3c4ccccc4c4cc5c6ccccc6n(-c6ccc(-c7ccccc7)cc6)c5cc43)cc2)cc1]

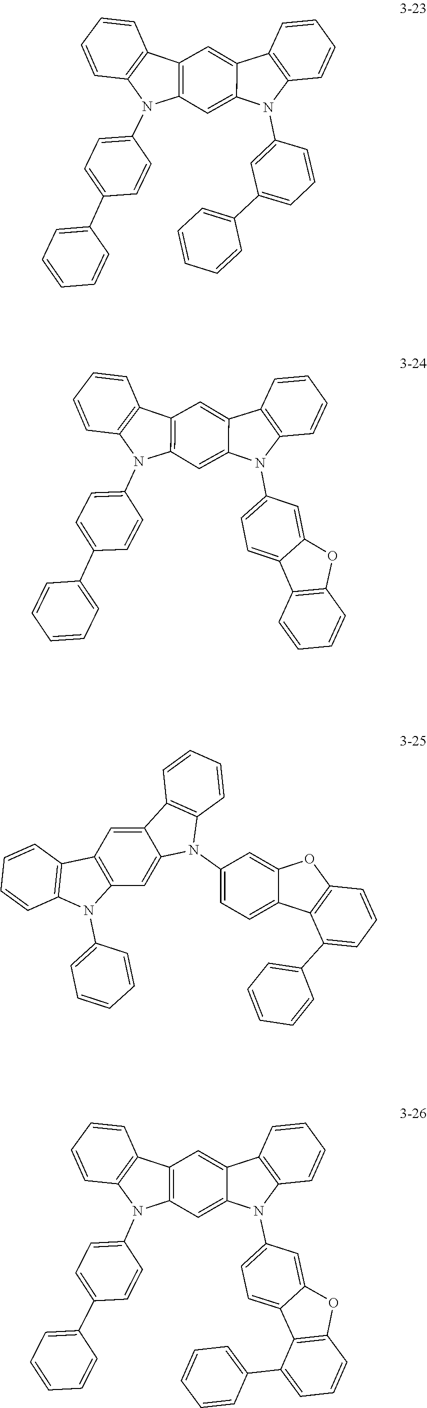 [SMILES: c1ccc(-c2ccc(-n3c4ccccc4c4cc5c6ccccc6n(-c6cccc(-c7ccccc7)c6)c5cc43)cc2)cc1 ; c1ccc(-c2ccc(-n3c4ccccc4c4cc5c6ccccc6n(-c6ccc7c(c6)oc6ccccc67)c5cc43)cc2)cc1 ; c1ccc(-c2cccc3oc4cc(-n5c6ccccc6c6cc7c8ccccc8n(-c8ccccc8)c7cc65)ccc4c23)cc1 ; c1ccc(-c2ccc(-n3c4ccccc4c4cc5c6ccccc6n(-c6ccc7c(c6)oc6cccc(-c8ccccc8)c67)c5cc43)cc2)cc1]

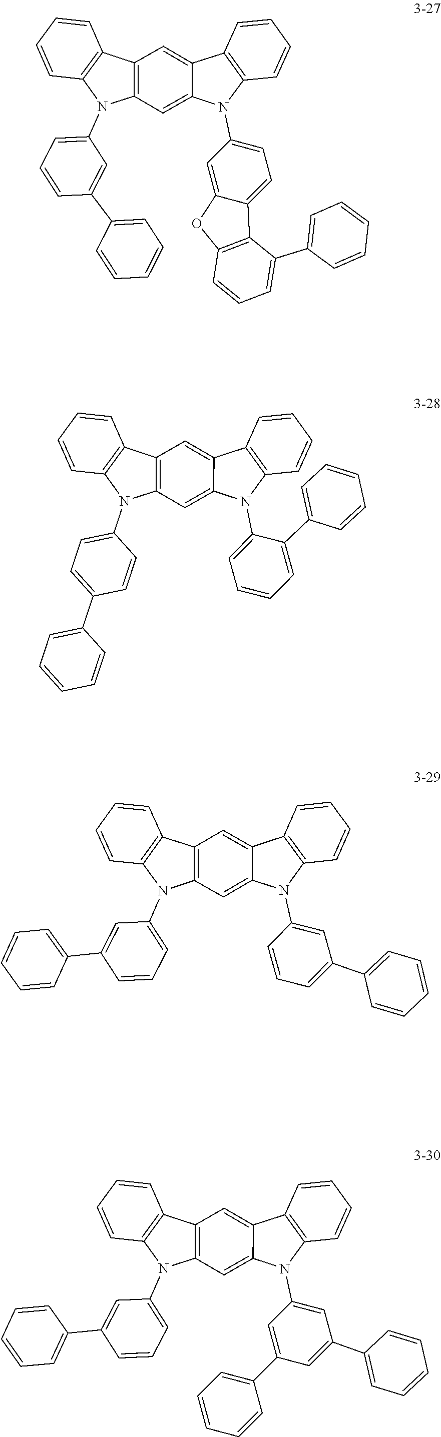 [SMILES: c1ccc(-c2cccc(-n3c4ccccc4c4cc5c6ccccc6n(-c6ccc7c(c6)oc6cccc(-c8ccccc8)c67)c5cc43)c2)cc1 ; c1ccc(-c2ccc(-n3c4ccccc4c4cc5c6ccccc6n(-c6ccccc6-c6ccccc6)c5cc43)cc2)cc1 ; c1ccc(-c2cccc(-n3c4ccccc4c4cc5c6ccccc6n(-c6cccc(-c7ccccc7)c6)c5cc43)c2)cc1 ; c1ccc(-c2cccc(-n3c4ccccc4c4cc5c6ccccc6n(-c6cc(-c7ccccc7)cc(-c7ccccc7)c6)c5cc43)c2)cc1]

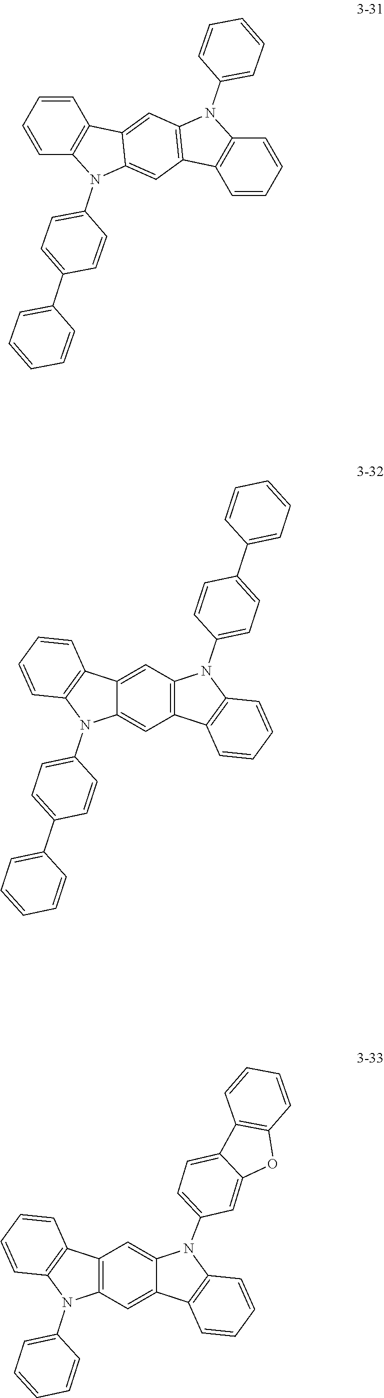 [SMILES: c1ccc(-c2ccc(-n3c4ccccc4c4cc5c(cc43)c3ccccc3n5-c3ccccc3)cc2)cc1 ; c1ccc(-c2ccc(-n3c4ccccc4c4cc5c(cc43)c3ccccc3n5-c3ccc(-c4ccccc4)cc3)cc2)cc1 ; c1ccc(-n2c3ccccc3c3cc4c(cc32)c2ccccc2n4-c2ccc3c(c2)oc2ccccc23)cc1]

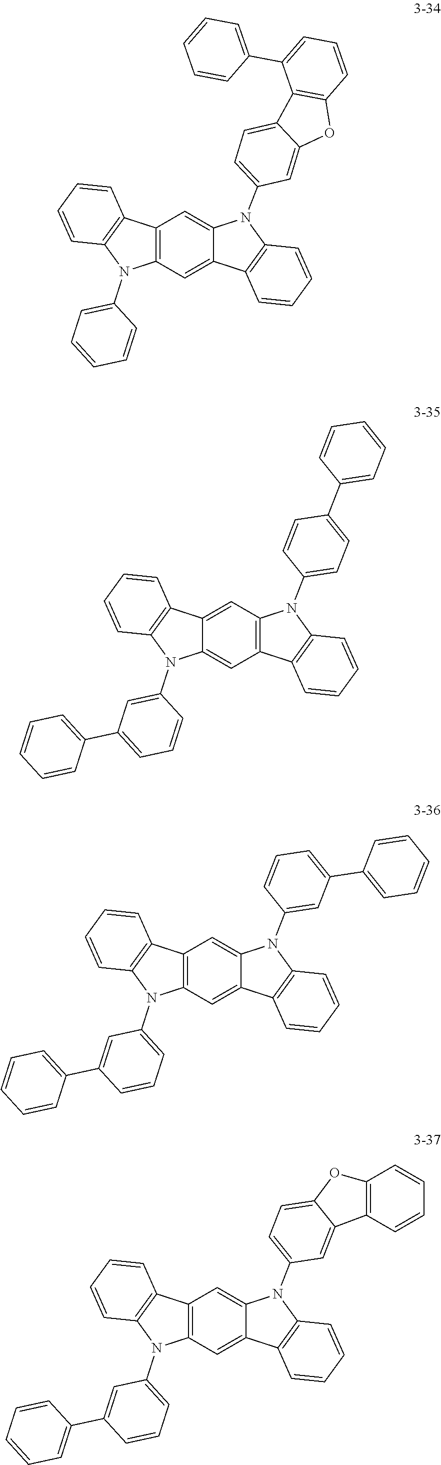 [SMILES: c1ccc(-c2cccc3oc4cc(-n5c6ccccc6c6cc7c(cc65)c5ccccc5n7-c5ccccc5)ccc4c23)cc1 ; c1ccc(-c2ccc(-n3c4ccccc4c4cc5c(cc43)c3ccccc3n5-c3cccc(-c4ccccc4)c3)cc2)cc1 ; c1ccc(-c2cccc(-n3c4ccccc4c4cc5c(cc43)c3ccccc3n5-c3cccc(-c4ccccc4)c3)c2)cc1 ; c1ccc(-c2cccc(-n3c4ccccc4c4cc5c(cc43)c3ccccc3n5-c3ccc4oc5ccccc5c4c3)c2)cc1]

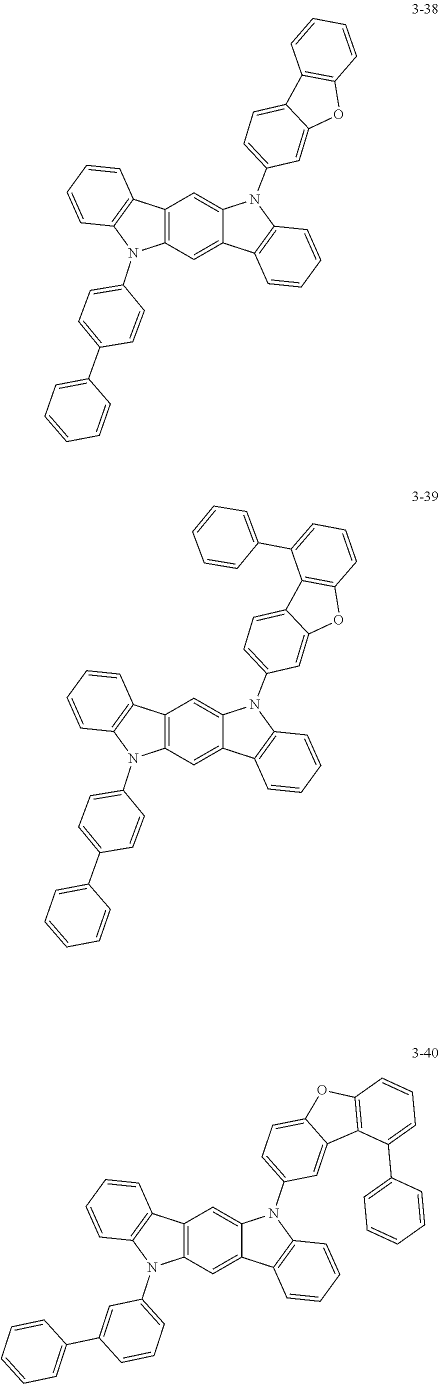 [SMILES: c1ccc(-c2ccc(-n3c4ccccc4c4cc5c(cc43)c3ccccc3n5-c3ccc4c(c3)oc3ccccc34)cc2)cc1 ; c1ccc(-c2ccc(-n3c4ccccc4c4cc5c(cc43)c3ccccc3n5-c3ccc4c(c3)oc3cccc(-c5ccccc5)c34)cc2)cc1 ; c1ccc(-c2cccc(-n3c4ccccc4c4cc5c(cc43)c3ccccc3n5-c3ccc4oc5cccc(-c6ccccc6)c5c4c3)c2)cc1]

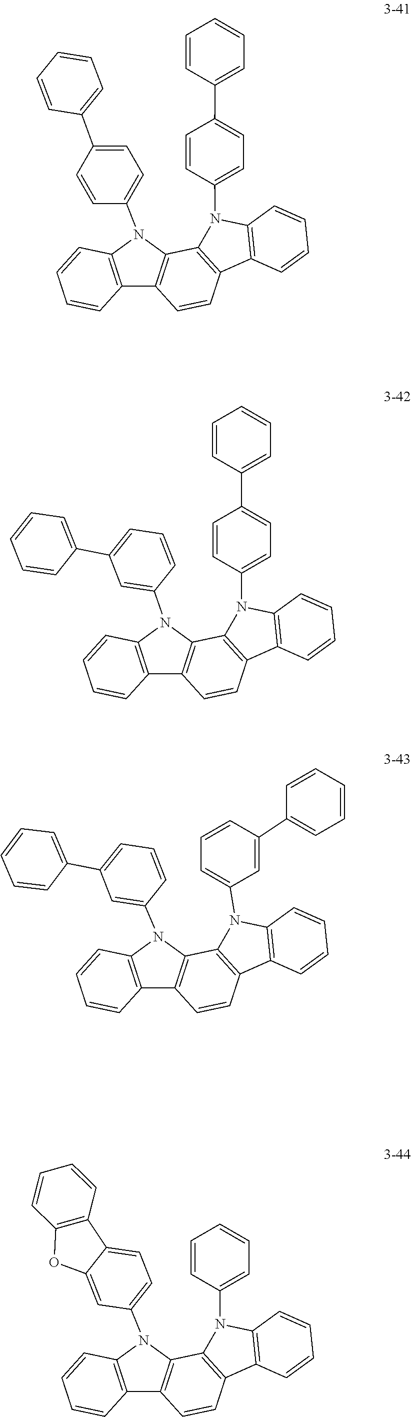 [SMILES: c1ccc(-c2ccc(-n3c4ccccc4c4ccc5c6ccccc6n(-c6ccc(-c7ccccc7)cc6)c5c43)cc2)cc1 ; c1ccc(-c2ccc(-n3c4ccccc4c4ccc5c6ccccc6n(-c6cccc(-c7ccccc7)c6)c5c43)cc2)cc1 ; c1ccc(-c2cccc(-n3c4ccccc4c4ccc5c6ccccc6n(-c6cccc(-c7ccccc7)c6)c5c43)c2)cc1 ; c1ccc(-n2c3ccccc3c3ccc4c5ccccc5n(-c5ccc6c(c5)oc5ccccc56)c4c32)cc1]

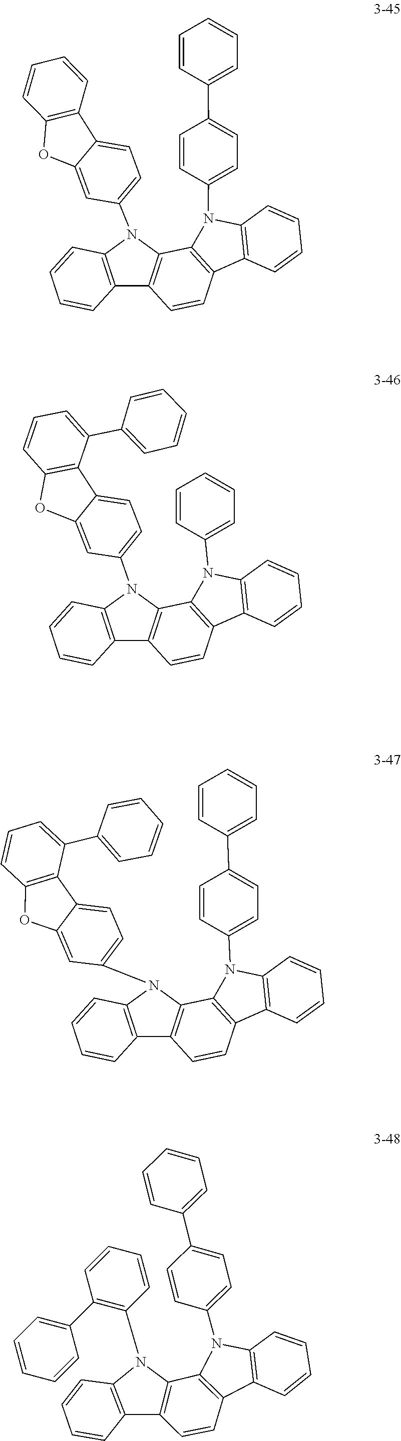 [SMILES: c1ccc(-c2ccc(-n3c4ccccc4c4ccc5c6ccccc6n(-c6ccc7c(c6)oc6ccccc67)c5c43)cc2)cc1 ; c1ccc(-c2cccc3oc4cc(-n5c6ccccc6c6ccc7c8ccccc8n(-c8ccccc8)c7c65)ccc4c23)cc1 ; c1ccc(-c2ccc(-n3c4ccccc4c4ccc5c6ccccc6n(-c6ccc7c(c6)oc6cccc(-c8ccccc8)c67)c5c43)cc2)cc1 ; c1ccc(-c2ccc(-n3c4ccccc4c4ccc5c6ccccc6n(-c6ccccc6-c6ccccc6)c5c43)cc2)cc1]

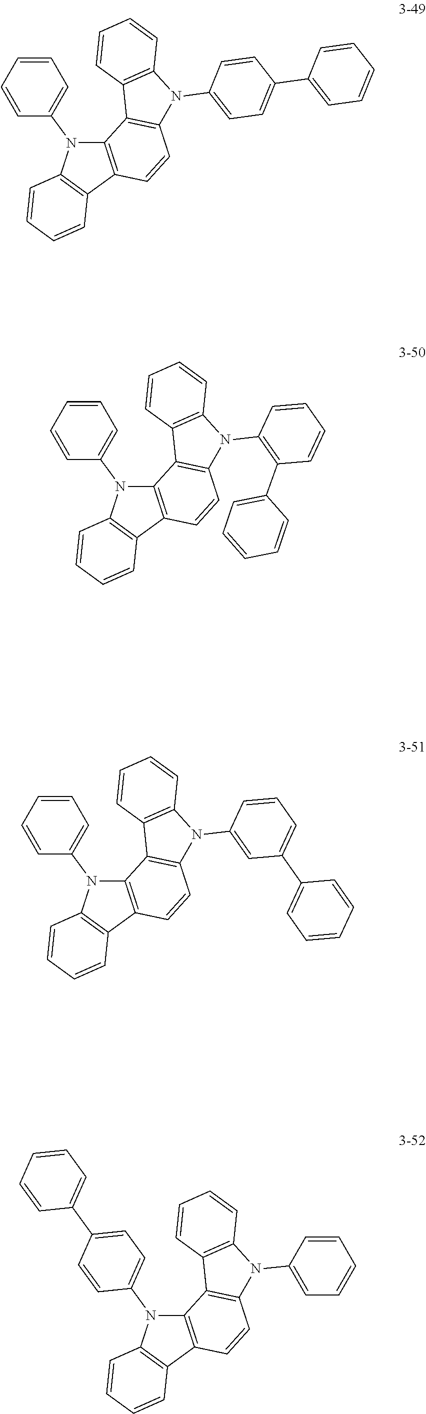 [SMILES: c1ccc(-c2ccc(-n3c4ccccc4c4c3ccc3c5ccccc5n(-c5ccccc5)c34)cc2)cc1 ; c1ccc(-c2ccccc2-n2c3ccccc3c3c2ccc2c4ccccc4n(-c4ccccc4)c23)cc1 ; c1ccc(-c2cccc(-n3c4ccccc4c4c3ccc3c5ccccc5n(-c5ccccc5)c34)c2)cc1 ; c1ccc(-c2ccc(-n3c4ccccc4c4ccc5c(c6ccccc6n5-c5ccccc5)c43)cc2)cc1]

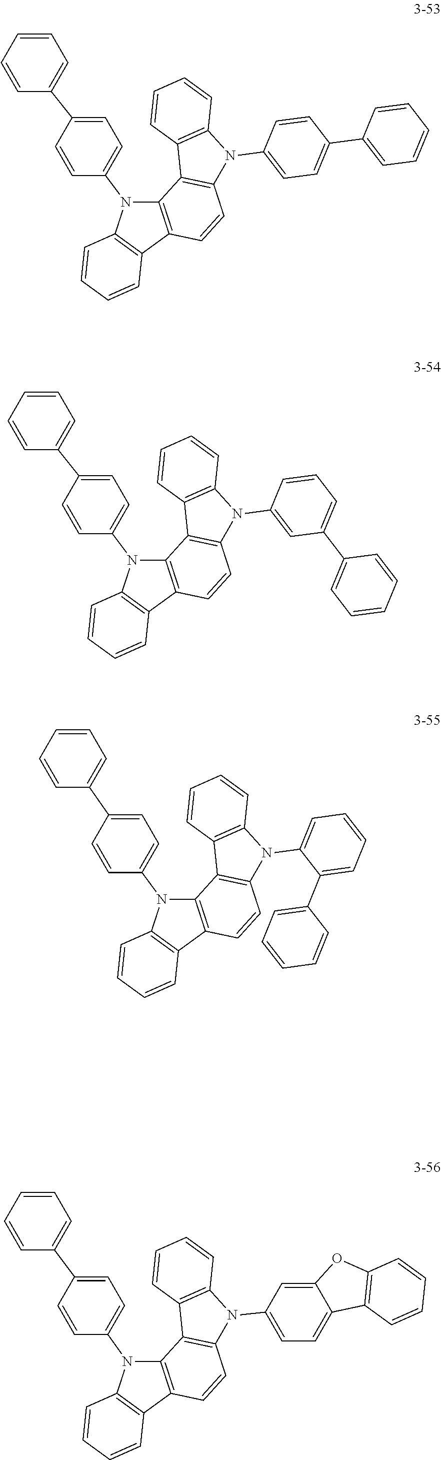 [SMILES: c1ccc(-c2ccc(-n3c4ccccc4c4c3ccc3c5ccccc5n(-c5ccc(-c6ccccc6)cc5)c34)cc2)cc1 ; c1ccc(-c2ccc(-n3c4ccccc4c4ccc5c(c6ccccc6n5-c5cccc(-c6ccccc6)c5)c43)cc2)cc1 ; c1ccc(-c2ccc(-n3c4ccccc4c4ccc5c(c6ccccc6n5-c5ccccc5-c5ccccc5)c43)cc2)cc1 ; c1ccc(-c2ccc(-n3c4ccccc4c4ccc5c(c6ccccc6n5-c5ccc6c(c5)oc5ccccc56)c43)cc2)cc1]

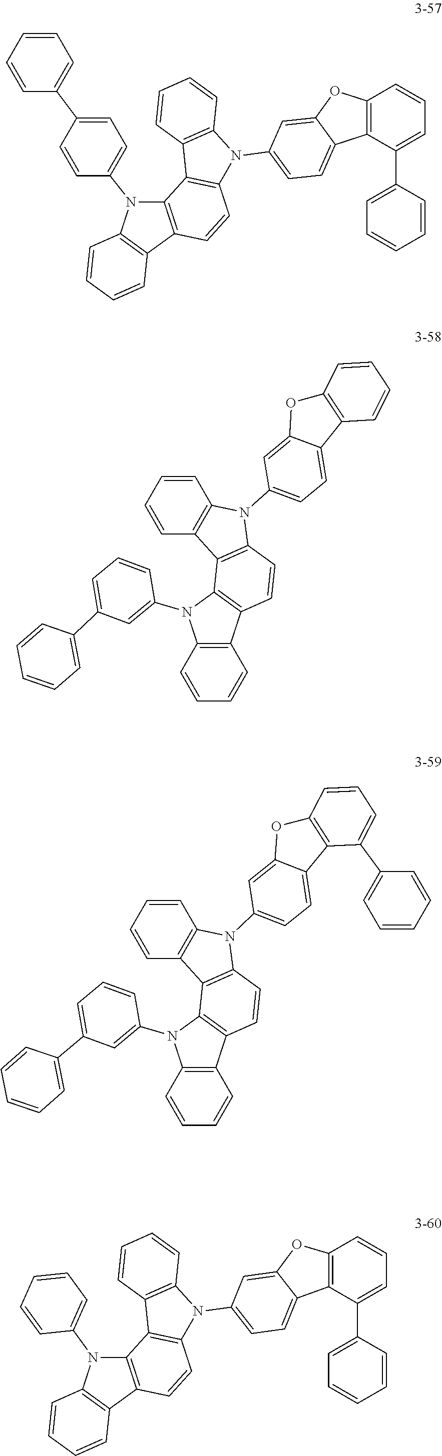 [SMILES: c1ccc(-c2ccc(-n3c4ccccc4c4ccc5c(c6ccccc6n5-c5ccc6c(c5)oc5cccc(-c7ccccc7)c56)c43)cc2)cc1 ; c1ccc(-c2cccc(-n3c4ccccc4c4ccc5c(c6ccccc6n5-c5ccc6c(c5)oc5ccccc56)c43)c2)cc1 ; c1ccc(-c2cccc(-n3c4ccccc4c4ccc5c(c6ccccc6n5-c5ccc6c(c5)oc5cccc(-c7ccccc7)c56)c43)c2)cc1 ; c1ccc(-c2cccc3oc4cc(-n5c6ccccc6c6c5ccc5c7ccccc7n(-c7ccccc7)c56)ccc4c23)cc1]

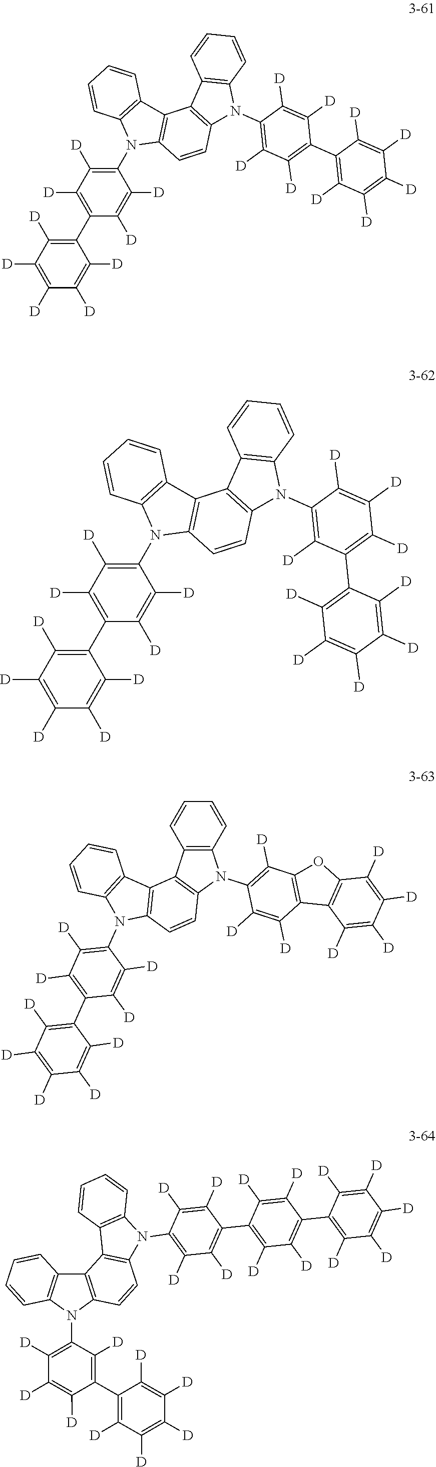

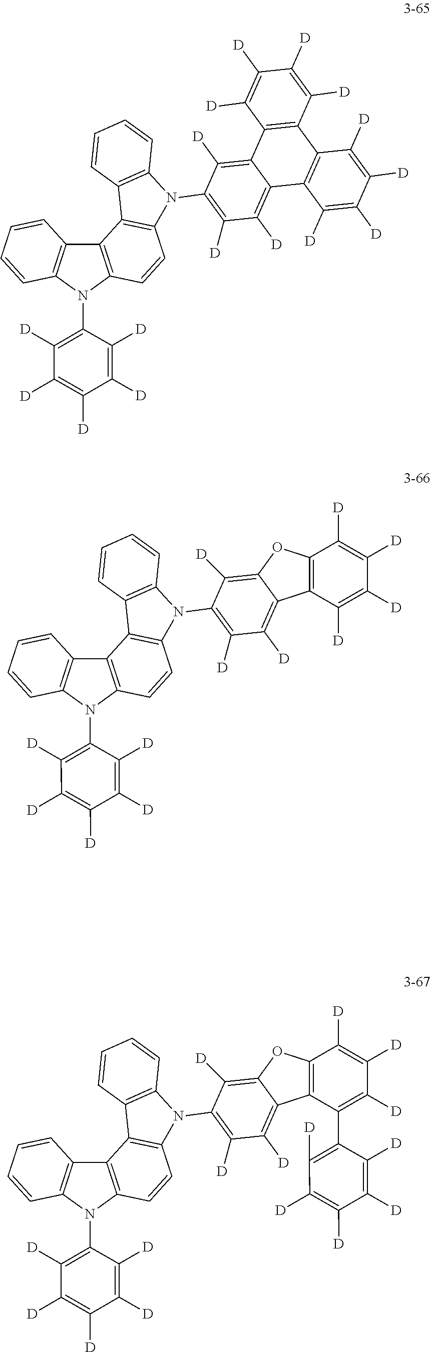 [SMILES: [2H]c1c([2H])c([2H])c(-n2c3ccccc3c3c4c5ccccc5n(-c5c([2H])c([2H])c6c7c([2H])c([2H])c([2H])c([2H])c7c7c([2H])c([2H])c([2H])c([2H])c7c6c5[2H])c4ccc32)c([2H])c1[2H] ; [2H]c1c([2H])c([2H])c(-n2c3ccccc3c3c4c5ccccc5n(-c5c([2H])c([2H])c6c(oc7c([2H])c([2H])c([2H])c([2H])c76)c5[2H])c4ccc32)c([2H])c1[2H] ; [2H]c1c([2H])c([2H])c(-c2c([2H])c([2H])c([2H])c3oc4c([2H])c(-n5c6ccccc6c6c7c8ccccc8n(-c8c([2H])c([2H])c([2H])c([2H])c8[2H])c7ccc65)c([2H])c([2H])c4c23)c([2H])c1[2H]]

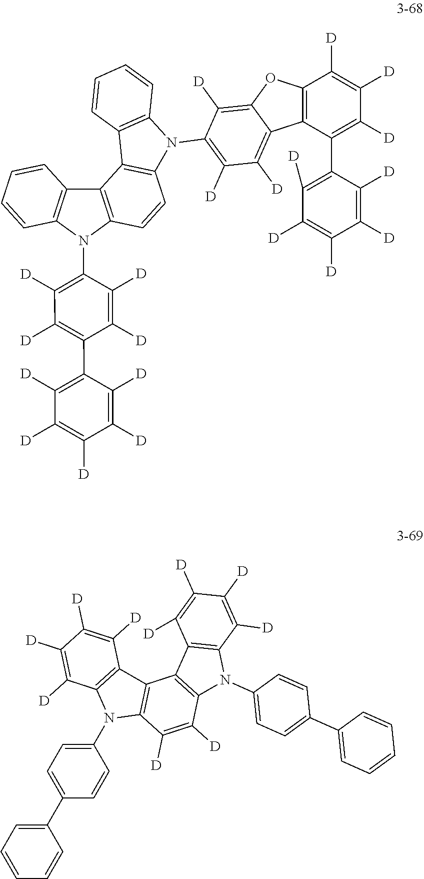 [SMILES: [2H]c1c([2H])c([2H])c(-c2c([2H])c([2H])c(-n3c4ccccc4c4c5c6ccccc6n(-c6c([2H])c([2H])c7c(oc8c([2H])c([2H])c([2H])c(-c9c([2H])c([2H])c([2H])c([2H])c9[2H])c87)c6[2H])c5ccc43)c([2H])c2[2H])c([2H])c1[2H] ; [2H]c1c([2H])c([2H])c2c(c1[2H])c1c3c4c([2H])c([2H])c([2H])c([2H])c4n(-c4ccc(-c5ccccc5)cc4)c3c([2H])c([2H])c1n2-c1ccc(-c2ccccc2)cc1]

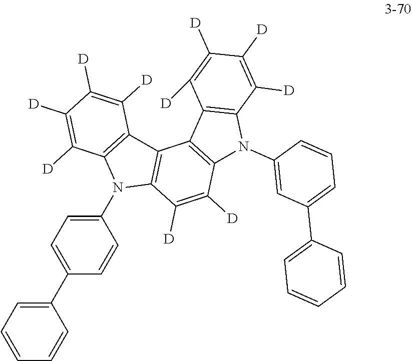 [SMILES: [2H]c1c([2H])c([2H])c2c(c1[2H])c1c3c4c([2H])c([2H])c([2H])c([2H])c4n(-c4cccc(-c5ccccc5)c4)c3c([2H])c([2H])c1n2-c1ccc(-c2ccccc2)cc1]

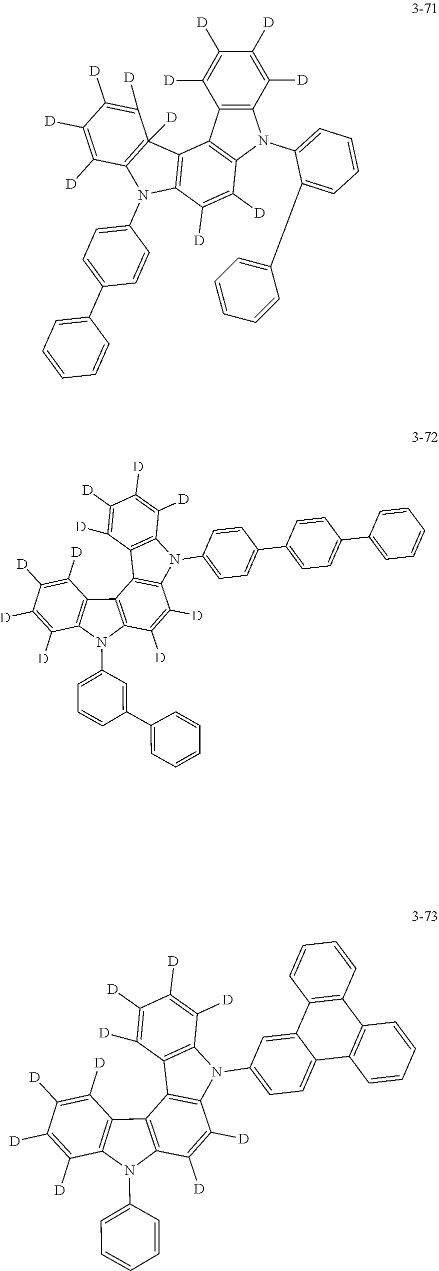

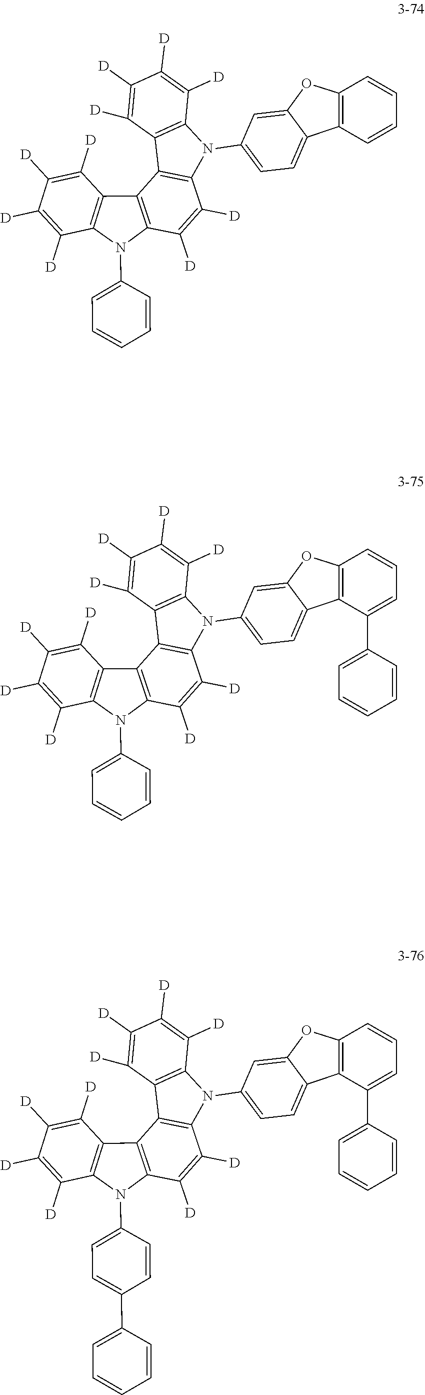 [SMILES: [2H]c1c([2H])c([2H])c2c(c1[2H])c1c3c4c([2H])c([2H])c([2H])c([2H])c4n(-c4ccc5c(c4)oc4ccccc45)c3c([2H])c([2H])c1n2-c1ccccc1 ; [2H]c1c([2H])c([2H])c2c(c1[2H])c1c3c4c([2H])c([2H])c([2H])c([2H])c4n(-c4ccc5c(c4)oc4cccc(-c6ccccc6)c45)c3c([2H])c([2H])c1n2-c1ccccc1 ; [2H]c1c([2H])c([2H])c2c(c1[2H])c1c3c4c([2H])c([2H])c([2H])c([2H])c4n(-c4ccc5c(c4)oc4cccc(-c6ccccc6)c45)c3c([2H])c([2H])c1n2-c1ccc(-c2ccccc2)cc1]

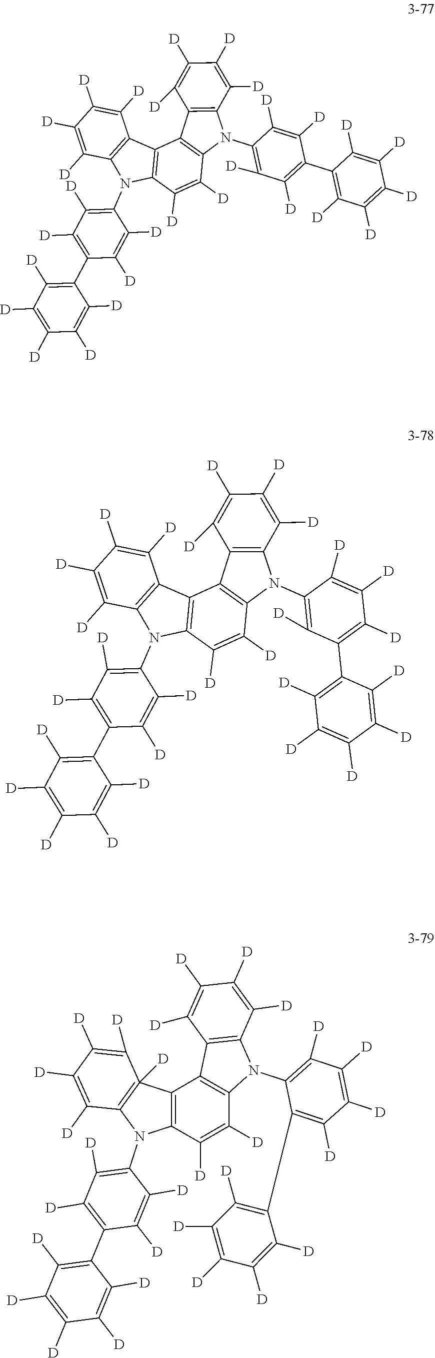

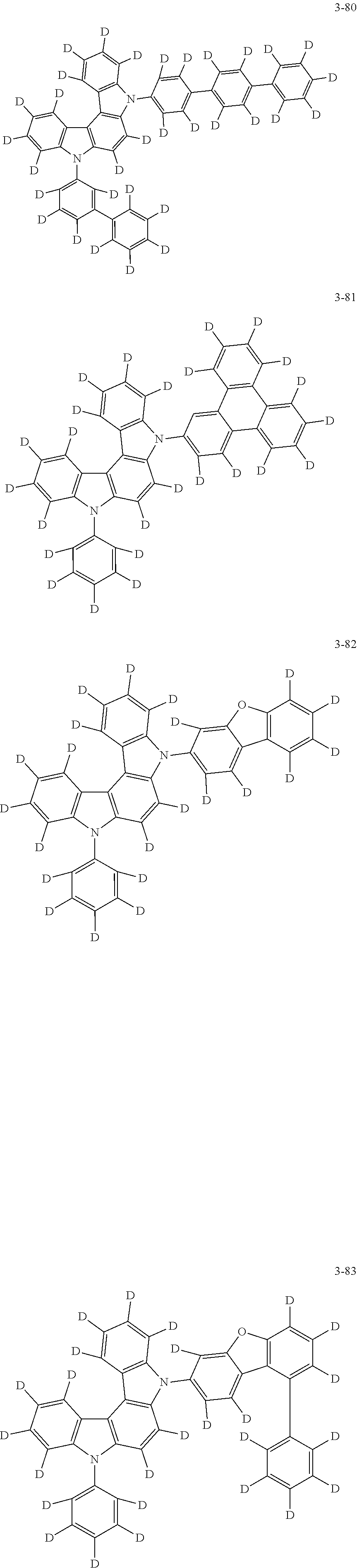

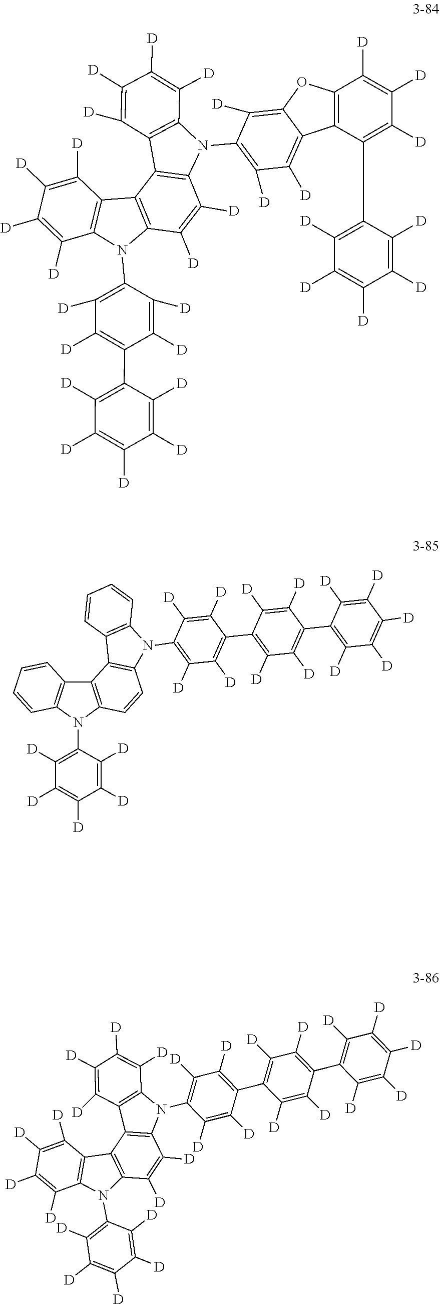

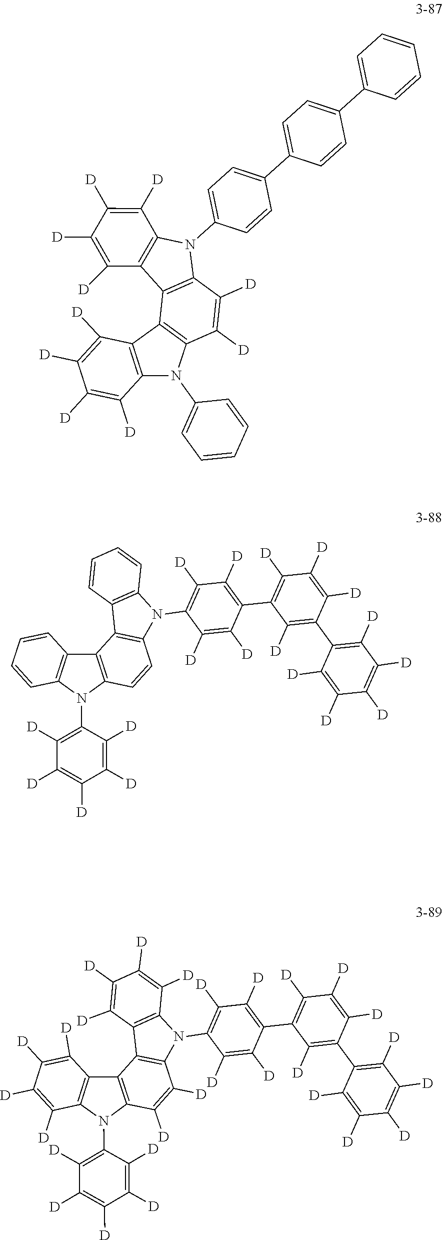

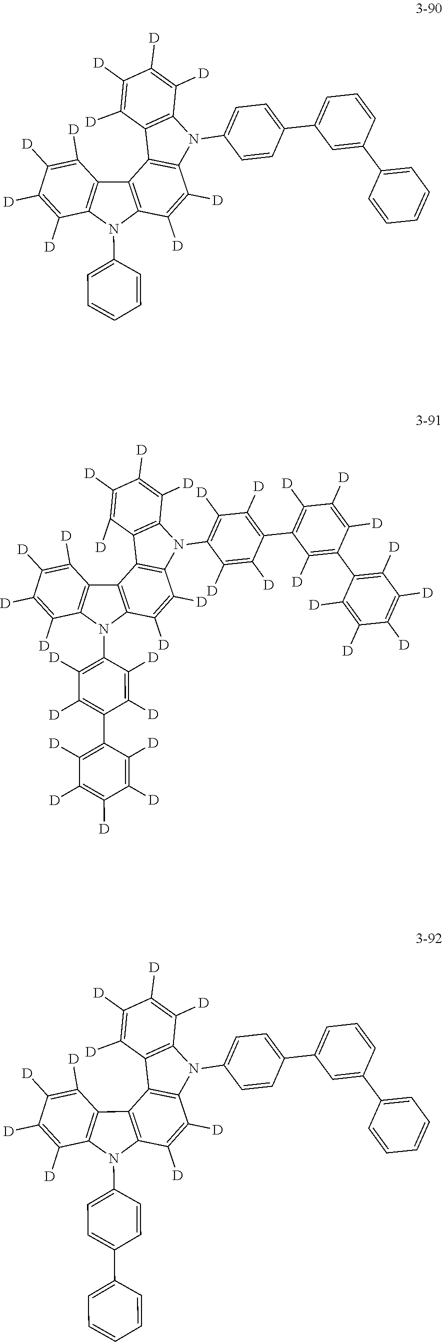

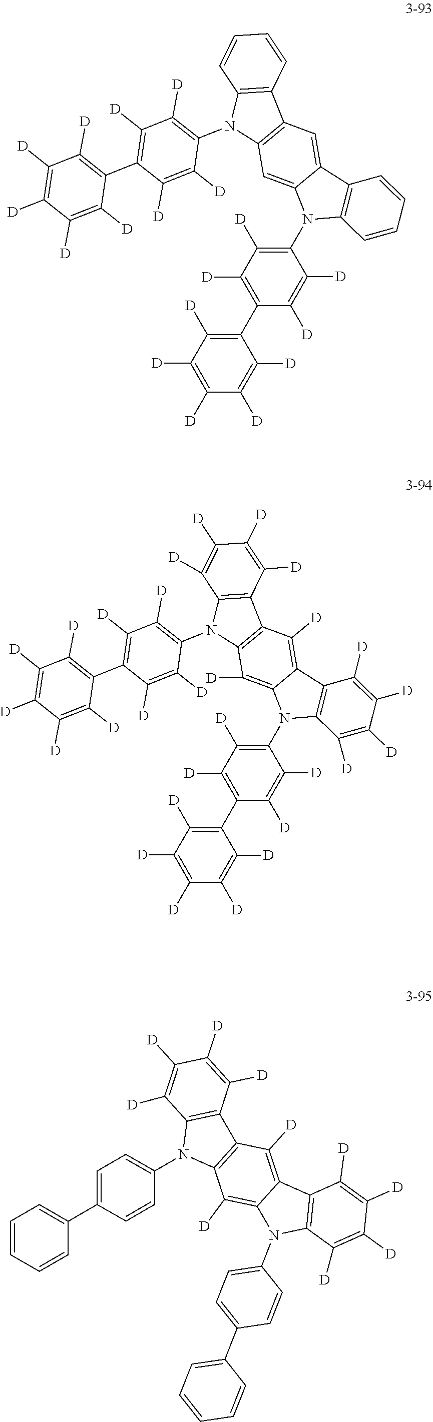

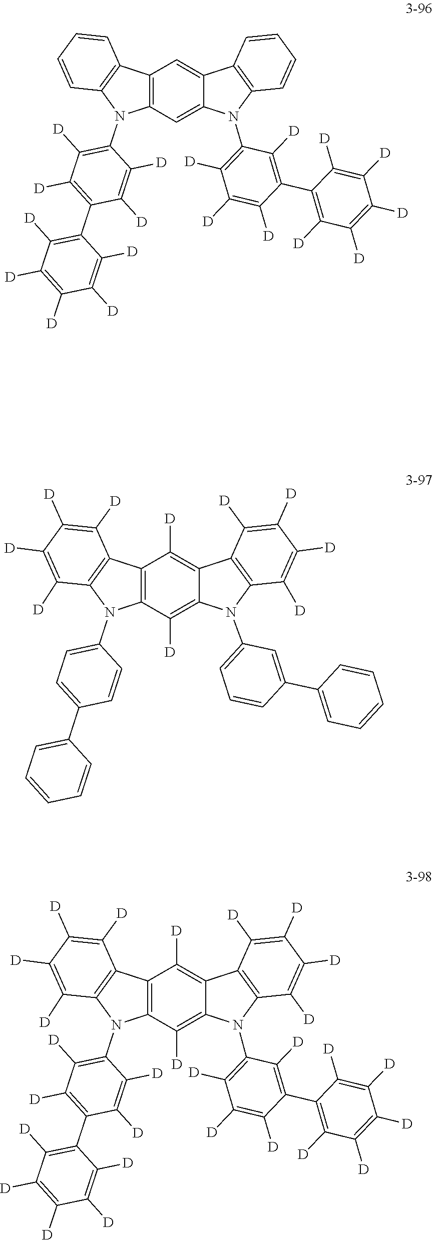

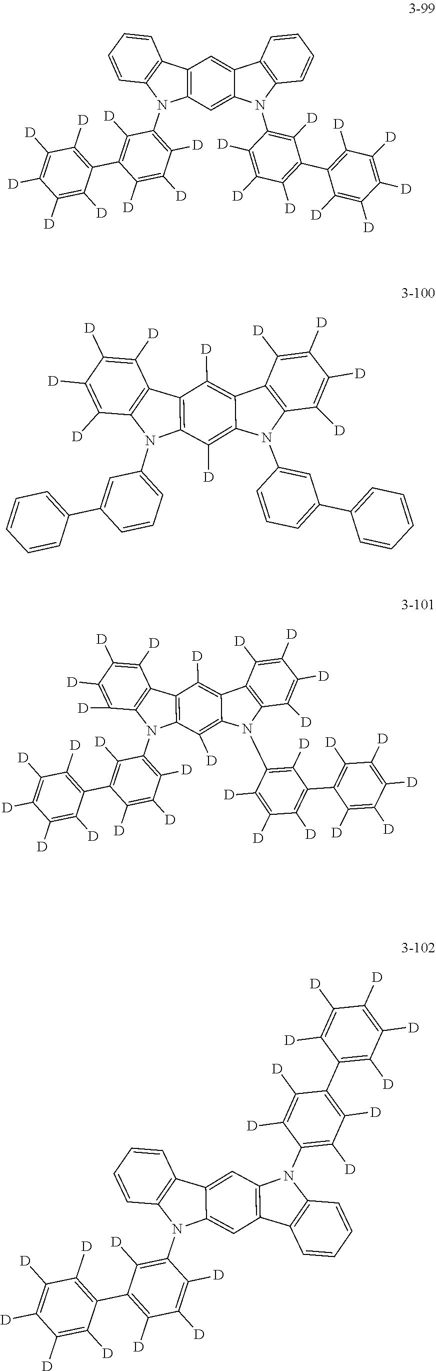

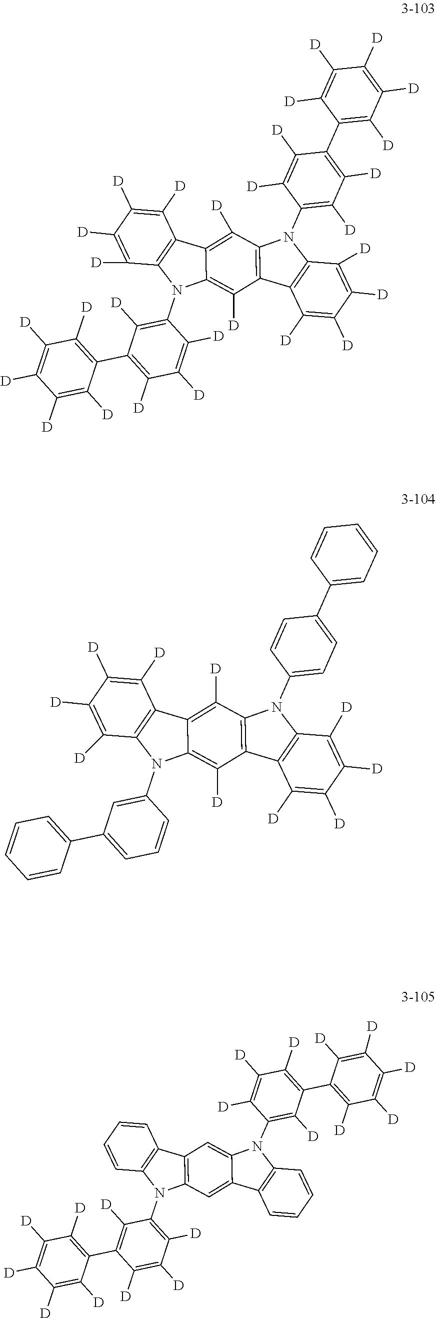

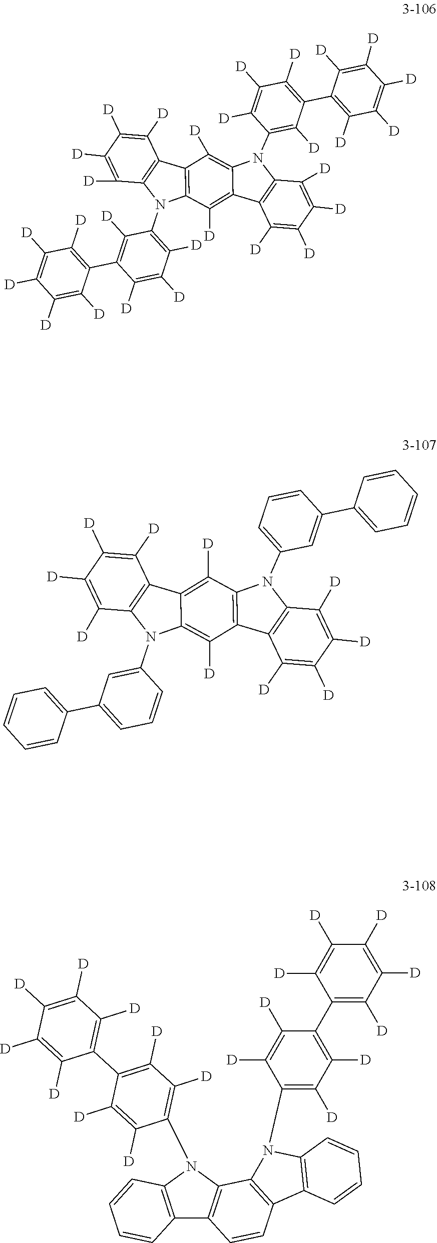

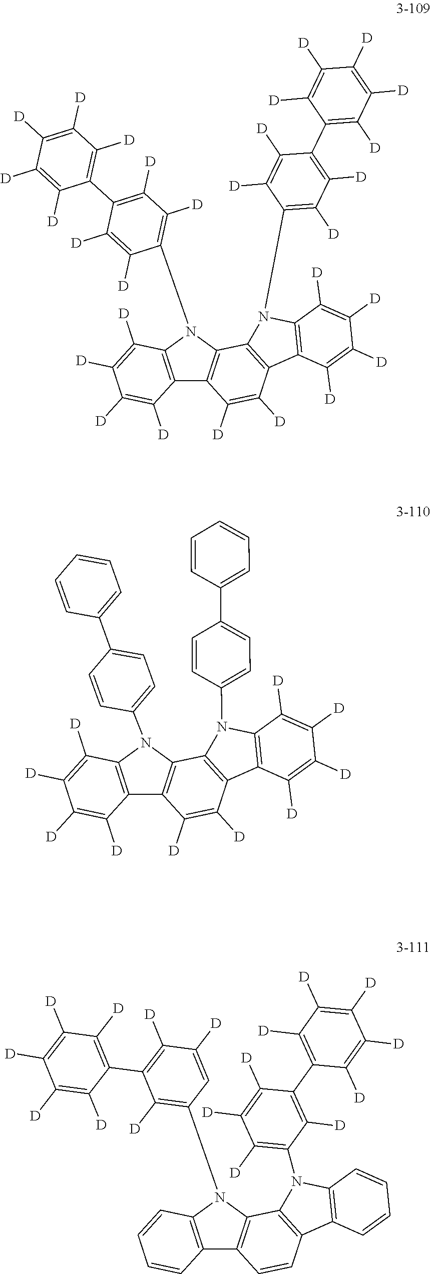

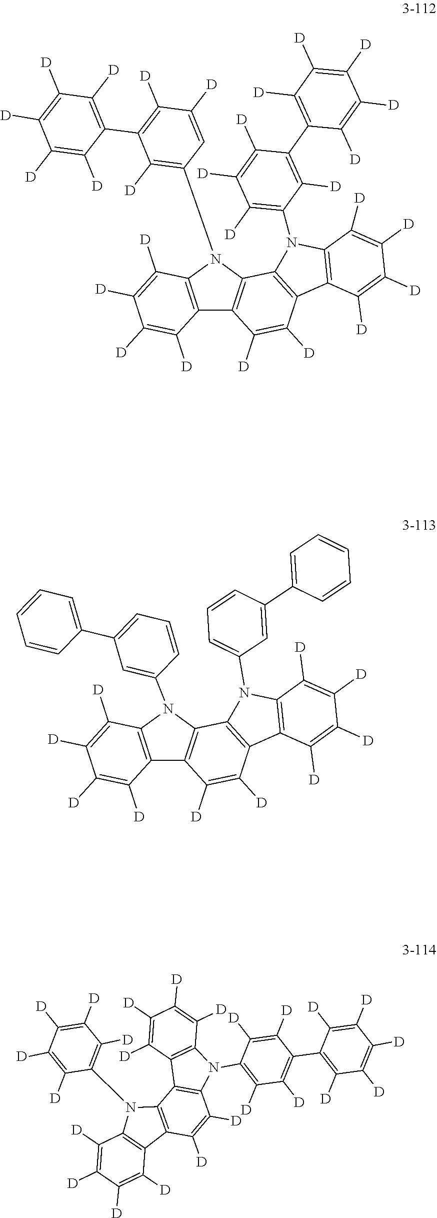

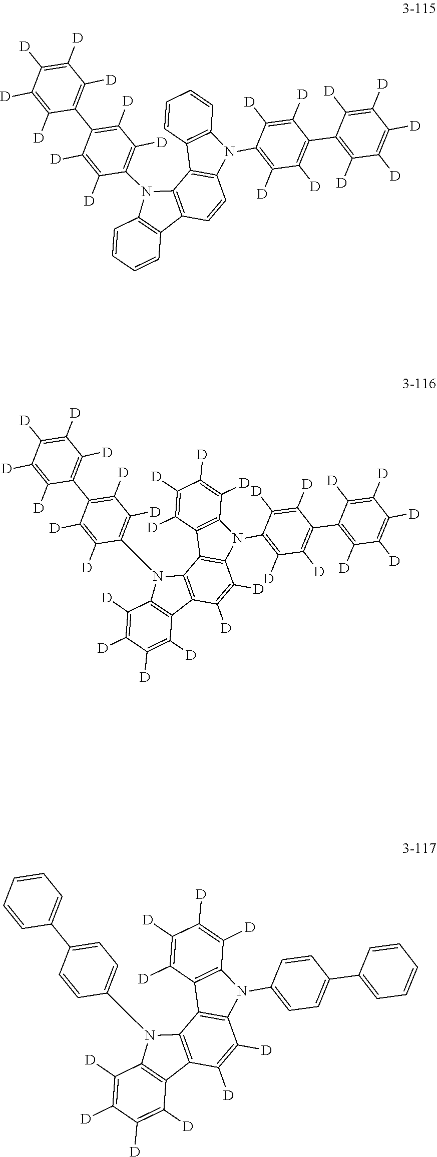

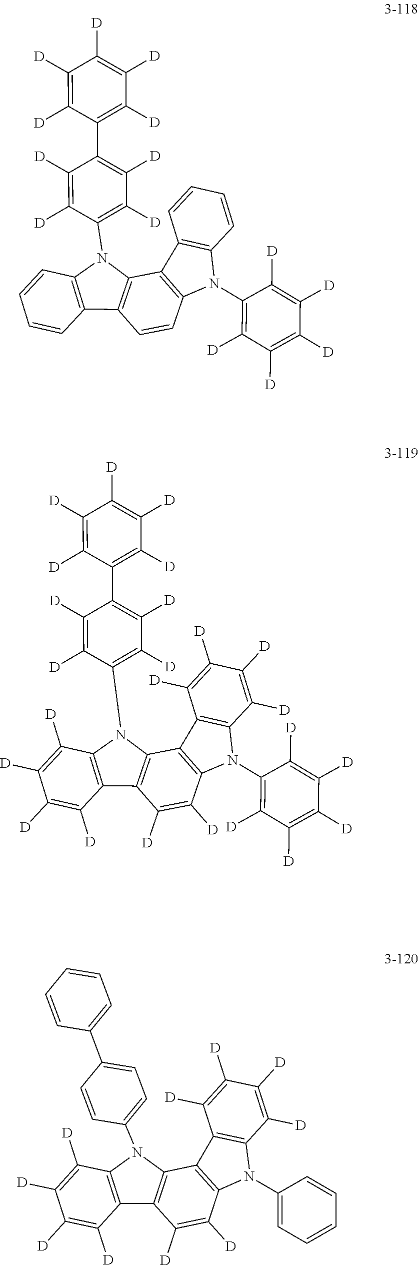

In addition, another embodiment of the present application provides a composition for an organic material layer, the composition including: the heterocyclic compound represented by Chemical Formula 1, and the heterocyclic compound represented by Chemical Formula 2 or 3.

Specific descriptions on the heterocyclic compound represented by Chemical Formula 1 and the heterocyclic compound represented by Chemical Formula 2 or 3 are the same as the descriptions provided above.

The heterocyclic compound represented by Chemical Formula 1 and the heterocyclic compound represented by Chemical Formula 2 or 3 may have a weight ratio of 1:10 to 10:1, 1:8 to 8:1, 1:5 to 5:1 or 1:2 to 2:1 in the composition, however, the ratio is not limited thereto.

The composition may be used when forming an organic material of an organic light emitting device, and particularly, may be more preferably used when forming a host of a light emitting layer.

The composition has a form in which two or more compounds are simply mixed, and materials in a powder state may be mixed before forming an organic material layer of an organic light emitting device, or compounds in a liquid state at a proper temperature or higher may be mixed. The composition is in a solid state at a melting point of each material or lower, and may be kept in a liquid state when adjusting a temperature.

The composition may further include materials known in the art such as solvents and additives.

The organic light emitting device according to one embodiment of the present application may be manufactured using common organic light emitting device manufacturing methods and materials except that one or more organic material layers are formed using the heterocyclic compound represented by Chemical Formula 1 and the heterocyclic compound represented by Chemical Formula 2 or 3 described above.

The compound represented by Chemical Formula 1 and the heterocyclic compound represented by Chemical Formula 2 or 3 may be formed into the organic material layer using a solution coating method as well as a vacuum deposition method when the organic light emitting device is manufactured. Herein, the solution coating method means spin coating, dip coating, inkjet printing, screen printing, a spray method, roll coating and the like, but is not limited thereto.

The organic material layer of the organic light emitting device of the present disclosure may be formed in a single layer structure, but may also be formed in a multilayer structure in which two or more organic material layers are laminated. For example, the organic light emitting device of the present disclosure may have a structure including a hole injection layer, a hole transport layer, a light emitting layer, an electron transport layer, an electron injection layer and the like as the organic material layer. However, the structure of the organic light emitting device is not limited thereto, and may include a smaller number of organic material layers.

In one embodiment of the present application, the organic light emitting device may be a blue organic light emitting device, and the heterocyclic compound according to Chemical Formula 1 and the heterocyclic compound according to Chemical Formula 2 or 3 may be used as a material of the blue organic light emitting device.

In one embodiment of the present application, the organic light emitting device may be a green organic light emitting device, and the compound represented by Chemical Formula 1 and the heterocyclic compound represented by Chemical Formula 2 or 3 may be used as a material of the green organic light emitting device.

In one embodiment of the present application, the organic light emitting device may be a red organic light emitting device, and the compound represented by Chemical Formula 1 and the heterocyclic compound represented by Chemical Formula 2 or 3 may be used as a material of the red organic light emitting device.

The organic light emitting device of the present disclosure may further include one, two or more layers selected from the group consisting of a light emitting layer, a hole injection layer, a hole transport layer, an electron injection layer, an electron transport layer, an electron blocking layer and a hole blocking layer.

One embodiment of the present application provides an organic light emitting device, wherein the organic material layer includes at least one of a hole blocking layer, an electron injection layer and an electron transport layer, and at least one of the hole blocking layer, the electron injection layer and the electron transport layer includes the heterocyclic compound represented by Chemical Formula 1 and the heterocyclic compound represented by Chemical Formula 2 or 3.

One embodiment of the present application provides an organic light emitting device, wherein the organic material layer includes a light emitting layer, and the light emitting layer includes the heterocyclic compound represented by Chemical Formula 1 and the heterocyclic compound represented by Chemical Formula 2 or 3.

One embodiment of the present application provides an organic light emitting device, wherein the organic material layer includes a light emitting layer, the light emitting layer includes a host material, and the host material includes the heterocyclic compound represented by Chemical Formula 1 and the heterocyclic compound represented by Chemical Formula 2 or 3.

FIGS. 1 to 3 illustrate a lamination order of electrodes and organic material layers of the organic light emitting device according to one embodiment of the present disclosure. However, it is not intended that the scope of the present application be limited by these drawings, and structures of organic light emitting devices known in the art may also be applied to the present application.

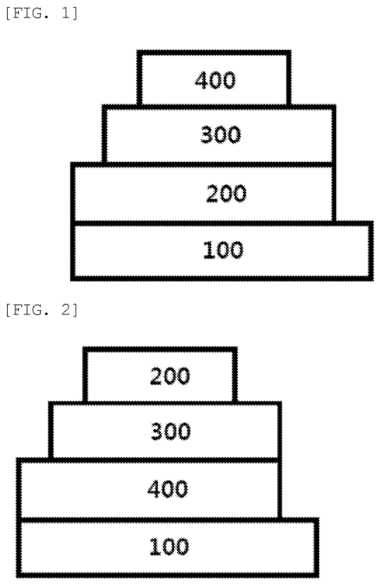

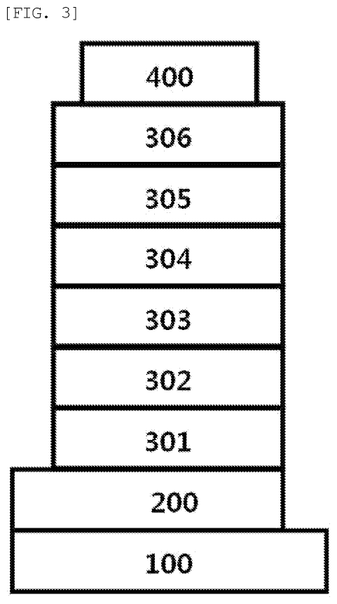

FIG. 1 illustrates an organic light emitting device in which a positive electrode 200, an organic material layer 300 and a negative electrode 400 are sequentially laminated on a substrate 100. However, the structure is not limited only to such a structure, and as illustrated in FIG. 2, an organic light emitting device in which a negative electrode, an organic material layer and a positive electrode are sequentially laminated on a substrate may also be obtained.

FIG. 3 illustrates a case of the organic material layer being a multilayer. An organic light emitting device according to FIG. 3 includes a hole injection layer 301, a hole transport layer 302, a light emitting layer 303, a hole blocking layer 304, an electron transport layer 305 and an electron injection layer 306. However, the scope of the present application is not limited by such a lamination structure, and as necessary, the layers other than the light emitting layer may not be included, and other necessary functional layers may be further added.

One embodiment of the present disclosure provides a method for manufacturing an organic light emitting device, the method including: preparing a substrate; forming a first electrode on the substrate; forming one or more organic material layers on the first electrode; and forming a second electrode on the organic material layers, wherein the forming of organic material layers includes forming one or more organic material layers using the composition for an organic material layer according to one embodiment of the present disclosure.

In one embodiment of the present disclosure, the forming of organic material layers may be forming organic material layers using a thermal vacuum deposition method after pre-mixing the heterocyclic compound represented by Chemical Formula 1.

The pre-mixing means, before depositing the heterocyclic compound represented by Chemical Formula 1 on the organic material layer, mixing the materials first in one source of supply.

The pre-mixed material may be referred to as the composition for an organic material layer according to one embodiment of the present application.

The organic material layer including the heterocyclic compound represented by Chemical Formula 1 may further include other materials as necessary.

In the organic light emitting device according to one embodiment of the present disclosure, materials other than the heterocyclic compound represented by Chemical Formula 1 are illustrated below, however, these are for illustrative purposes only and not for limiting the scope of the present application, and these materials may be replaced by materials known in the art.

As the positive electrode material, materials each having a relatively large work function may be used, and transparent conductive oxides, metals, conductive polymers or the like may be used. Specific examples of the positive electrode material include metals such as vanadium, chromium, copper, zinc and gold, or alloys thereof; metal oxides such as zinc oxide, indium oxide, indium tin oxide (ITO) and indium zinc oxide (IZO); combinations of metals and oxides such as Zno:Al or SnO₂:Sb; conductive polymers such as poly(3-methylthiophene), poly[3,4-(ethylene-1,2-dioxy)thiophene] (PEDOT), polypyrrole and polyaniline, and the like, but are not limited thereto.

As the negative electrode material, materials each having a relatively small work function may be used, and metals, metal oxides, conductive polymers or the like may be used. Specific examples of the negative electrode material include metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection layer material, known hole injection layer materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′,4″-tri[phenyl(m-tolyl)amino]triphenylamine (m-MTDATA) or 1,3,5-tris[4-(3-methylphenylphenylamino)phenyl]benzene (m-MTDAPB) described in the literature [Advanced Material, 6, p. 677 (1994)], conductive polymers having solubility such as polyaniline/dodecylbenzenesulfonic acid or poly(3,4-ethylenedioxythiophene)/poly(4-styrenesulfonate), polyaniline/camphor sulfonic acid or polyaniline/poly(4-styrenesulfonate), and the like, may be used.

As the hole transport layer material, pyrazoline derivatives, arylamine-based derivatives, stilbene derivatives, triphenyldiamine derivatives and the like may be used, and low molecular or high molecular materials may also be used.

As the electron transport layer material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials as well as low molecular materials may also be used.

As examples of the electron injection layer material, LiF is typically used in the art, however, the present application is not limited thereto.

As the light emitting layer material, red, green or blue light emitting materials may be used, and as necessary, two or more light emitting materials may be mixed and used. Herein, the two or more light emitting materials may be deposited as individual sources of supply or pre-mixed and deposited as one source of supply when used. In addition, fluorescent materials may also be used as the light emitting layer material, however, phosphorescent materials may also be used. As the light emitting layer material, materials emitting light alone by binding holes and electrons injected from a positive electrode and a negative electrode, respectively, may be used, however, materials having a host material and a dopant material involved in light emission together may also be used.

When hosts of the light emitting layer material are mixed and used, same series hosts may be mixed and used, or different series hosts may be mixed and used. For example, any two or more types of materials among n-type host materials and p-type host materials may be selected and used as a host material of a light emitting layer.

The organic light emitting device according to one embodiment of the present disclosure may be a top-emission type, a bottom-emission type or a dual-emission type depending on the materials used.

The heterocyclic compound according to one embodiment of the present disclosure may also be used in an organic electronic device including an organic solar cell, an organic photo conductor, an organic transistor and the like under a principle similar to that in the organic light emitting device.

Hereinafter, preferred examples are provided to help to understand the present disclosure, however, the following examples are only provided to more readily understand the present disclosure, and the present disclosure is not limited thereto.

### PREPARATION EXAMPLE

### <Preparation Example 1> Preparation of Compound 1-1

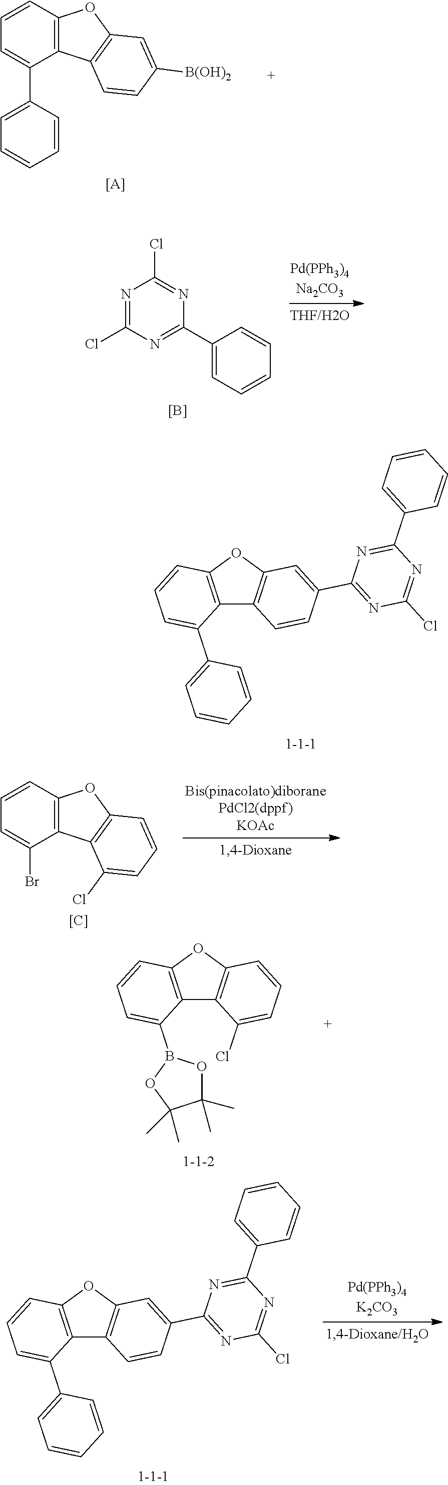

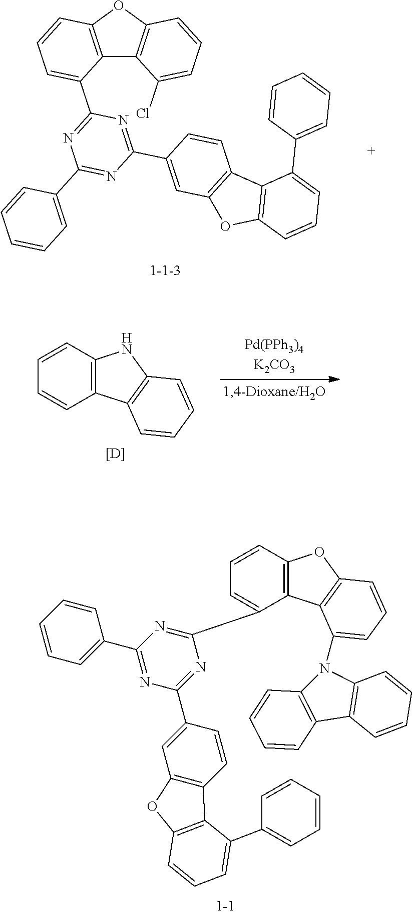

### 1) Preparation of Compound 1-1-1

In a one-neck round bottom flask, a mixture of (9-phenyldibenzo[b,d]furan-3-yl)boronic acid (8.36 g, 0.029 mol), 2,4-dichloro-6-phenyl-1,3,5-triazine (6.6 g, 0.029 mol), Pd(PPh₃)₄ (1.68 g, 0.0015 mol), Na₂CO₃ (6.15 g, 0.058 mol) and THE (80 mL)/H₂O (20 mL) was refluxed at 100° C. After the reaction was completed, the result was cooled and then extracted, and the organic layer was silica gel filtered to obtain Compound 1-1-1 (8.81 g, yield 70%).

### 2) Preparation of Compound 1-1-2

In a one-neck round bottom flask, a mixture of 1-bromo-9-chlorodibenzo[b,d]furan (8.81 g, 0.020 mol), bis(pinacolato)diboron (10.16 g, 0.040 mol), PdCl₂(dppf) (0.73 g, 0.001 mol), potassium acetate (3.89 g, 0.040 mol) and 1,4-dioxane (80 mL) was refluxed at 120° C. After the reaction was completed, the result was cooled, then concentrated, and silica gel filtered to obtain Compound 1-1-2 (6.24 g, yield 95%).

### 3) Preparation of Compound 1-1-3

In a one-neck round bottom flask, a mixture of 2-(9-chlorodibenzo[b,d]furan-1-yl)-4,4,5,5-tetramethyl-1,3,2-dioxaborolane [Compound 1-1-2] (6.24 g, 0.019 mol), 2-chloro-4-phenyl-6-(9-phenyldibenzo[b,d]furan-3-yl)-1,3,5-triazine [Compound 1-1-1] (8.24 g, 0.019 mol), Pd(PPh₃)₄ (1.10 g, 0.00095 mol), K₂CO₃ (5.25 g, 0.038 mol) and 1,4-dioxane (60 mL)/H₂O (20 mL) was refluxed at 120° C. After the reaction was completed, the result was cooled, then concentrated, and silica gel filtered to obtain Compound 1-1-3 (8.55 g, yield 75%).

### 4) Preparation of Compound 1-1

In a one-neck round bottom flask, a mixture of 2-(9-chlorodibenzo[b,d]furan-1-yl)-4-phenyl-6-(9-phenyldibenzo[b,d]furan-3-yl)-1,3,5-triazine [Compound 1-1-3] (8.55 g, 0.014 mol), 9H-carbazole (2.34 g, 0.014 mol), Pd(PPh₃)₄ (0.81 g, 0.0007 mol), K₂CO₃ (3.87 g, 0.028 mol) and 1,4-dioxane (80 mL)/H₂O (25 mL) was refluxed at 120° C. After the reaction was completed, the result was cooled, then concentrated, and silica gel filtered to obtain Compound 1-1 (6.24 g, yield 61%).

### <Preparation Example 2> Preparation of Compounds 1-1, 1-9, 1-12, 1-25, 1-28, 1-30, 1-34, 1-36, 1-39, 1-42, 1-46, 1-50, 1-52, 1-56, 1-58, 1-60, 1-81, 1-85, 1-87, 1-90, 1-92, 1-95, 1-98, 1-100, 1-103, 1-106, 1-109, 1-115, 1-120, 1-137, 1-144, 1-148, 1-154, 1-158, 1-161, 1-166, 1-176, 1-193, 1-195, 1-197, 1-198, 1-201, 1-205, 1-208, 1-209, 1-210, 1-213, 1-216, 1-217, 1-219, 1-222, 1-224, 1-226, 1-227, 1-232, 1-249, 1-251, 1-253, 1-256, 1-257, 1-260, 1-263, 1-266, 1-270, 1-273, 1-274, 1-276, 1-279, 1-280, 1-287, 1-301, 1-302, 1-305, 1-310, 1-312, 1-313, 1-316, 1-320, 1-321, 1-324, 1-326, 1-330, 1-333, 1-337, 1-355, 1-356, 1-360, 1-364, 1-368, 1-372, 1-373, 1-379, 1-381 and 1-388

Compounds 1-1, 1-9, 1-12, 1-25, 1-28, 1-30, 1-34, 1-36, 1-39, 1-42, 1-46, 1-50, 1-52, 1-56, 1-58, 1-60, 1-81, 1-85, 1-87, 1-90, 1-92, 1-95, 1-98, 1-100, 1-103, 1-106, 1-109, 1-115, 1-120, 1-137, 1-144, 1-148, 1-154, 1-158, 1-161, 1-166, 1-176, 1-193, 1-195, 1-197, 1-198, 1-201, 1-205, 1-208, 1-209, 1-210, 1-213, 1-216, 1-217, 1-219, 1-222, 1-224, 1-226, 1-227, 1-232, 1-249, 1-251, 1-253, 1-256, 1-257, 1-260, 1-263, 1-266, 1-270, 1-273, 1-274, 1-276, 1-279, 1-280, 1-287, 1-301, 1-302, 1-305, 1-310, 1-312, 1-313, 1-316, 1-320, 1-321, 1-324, 1-326, 1-330, 1-333, 1-337, 1-355, 1-356, 1-360, 1-364, 1-368, 1-372, 1-373, 1-379, 1-381 and 1-388 were synthesized in the same manner as in Preparation Example 1 except that Intermediates A′ to D′ of the following Table 1 were used instead of A to D.

TABLE 1

| Compound | A′ | B′ | C′ | D′ |
|---|---|---|---|---|
| 1-1 | 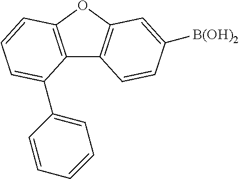 | 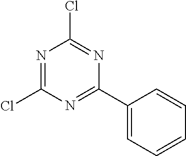 | 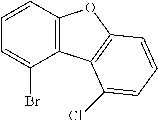 | 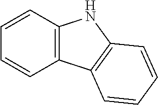 |
| 1-9 | 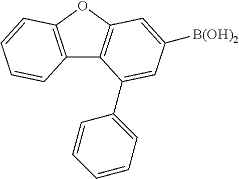 | 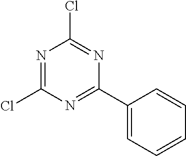 | 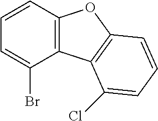 | 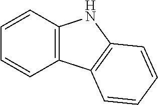 |
| 1-12 | 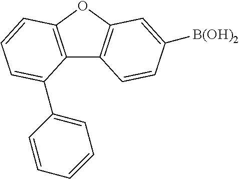 | 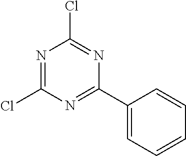 | 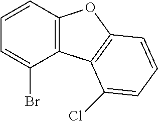 | 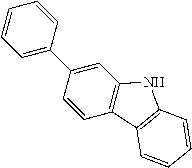 |
| 1-25 | 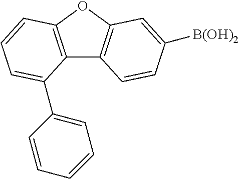 | 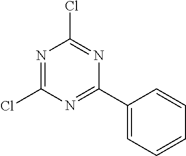 | 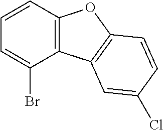 | 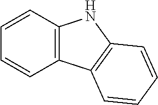 |
| 1-28 | 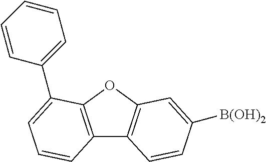 | 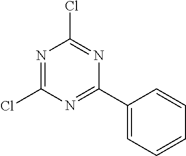 | 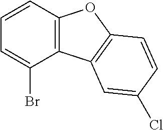 | 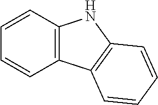 |
| 1-30 | 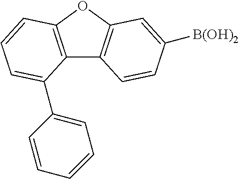 | 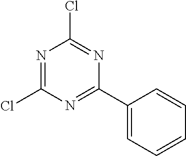 | 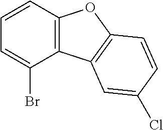 | 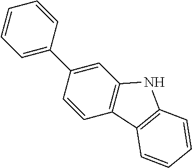 |
| 1-34 | 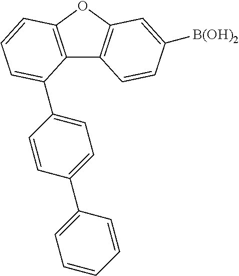 | 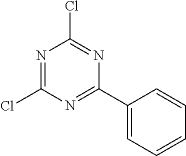 | 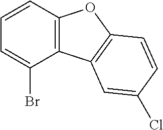 | 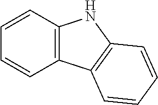 |
| 1-36 | 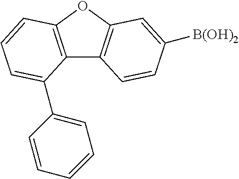 | 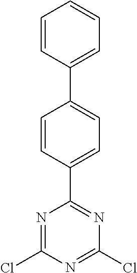 | 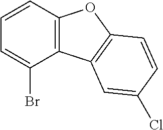 | 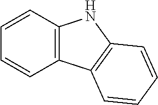 |
| 1-39 | 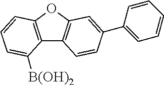 | 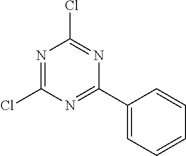 | 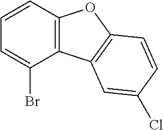 | 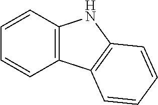 |
| 1-42 | 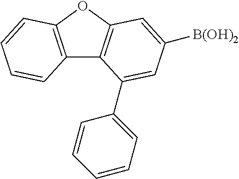 | 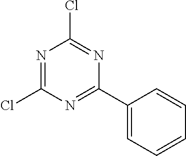 | 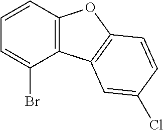 | 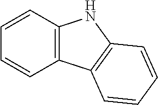 |
| 1-46 | 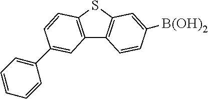 | 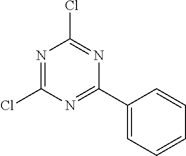 | 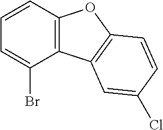 | 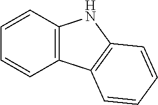 |
| 1-50 | 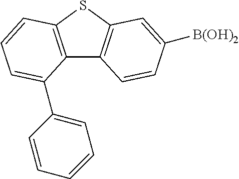 | 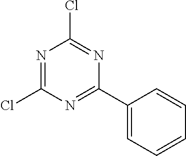 | 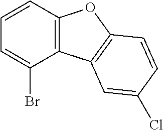 | 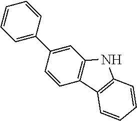 |
| 1-52 | 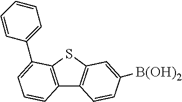 | 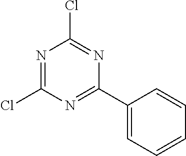 | 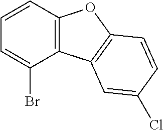 | 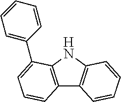 |
| 1-56 | 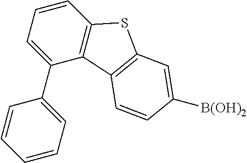 | 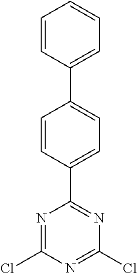 | 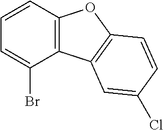 | 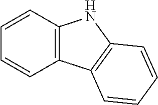 |
| 1-58 | 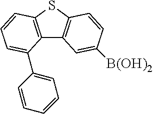 | 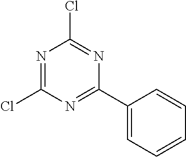 | 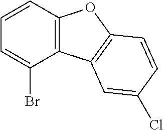 | 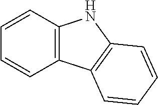 |
| 1-60 | 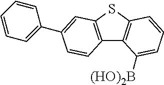 | 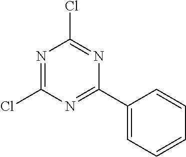 | 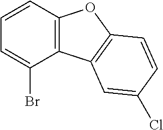 | 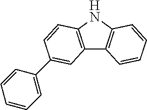 |
| 1-81 | 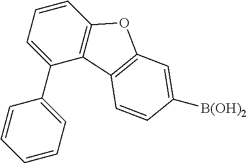 | 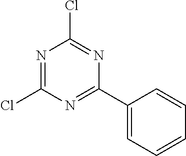 | 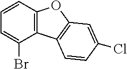 | 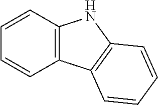 |
| 1-85 | 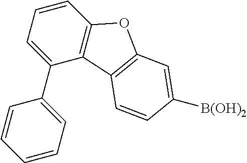 | 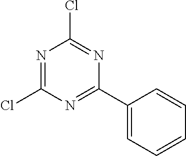 | 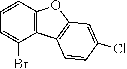 | 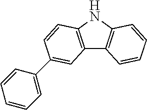 |
| 1-87 | 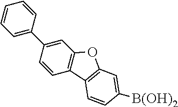 | 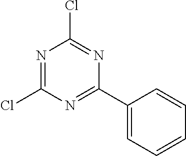 | 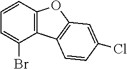 | 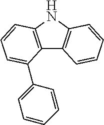 |
| 1-90 | 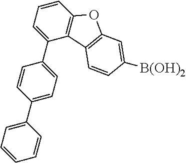 | 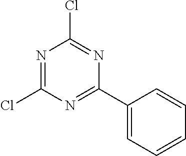 | 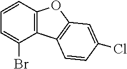 | 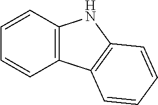 |
| 1-92 | 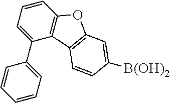 | 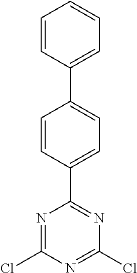 | 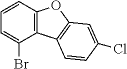 | 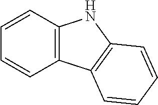 |
| 1-95 | 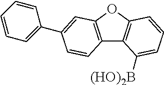 | 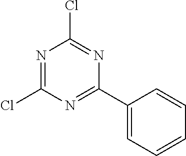 | 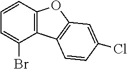 | 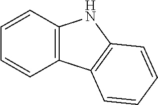 |
| 1-98 | 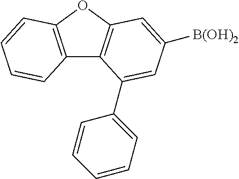 | 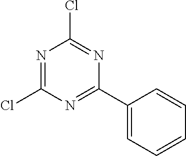 | 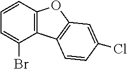 | 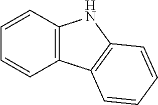 |
| 1-100 | 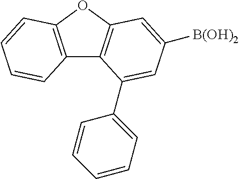 | 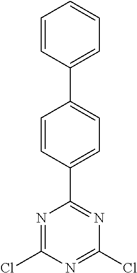 | 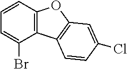 | 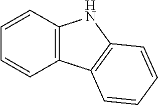 |
| 1-103 | 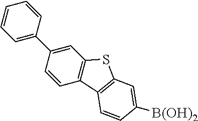 | 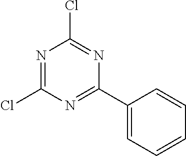 | 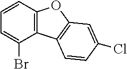 | 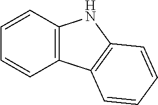 |
| 1-106 | 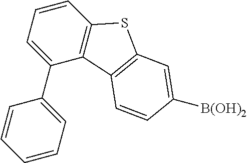 | 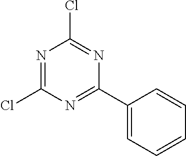 | 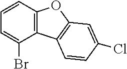 | 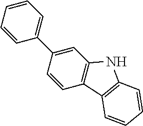 |
| 1-109 | 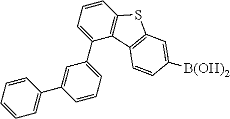 | 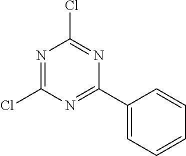 | 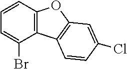 | 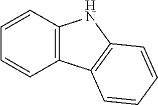 |
| 1-115 | 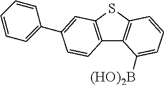 | 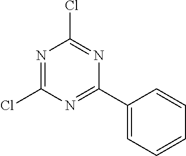 | 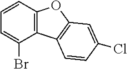 | 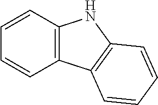 |
| 1-120 | 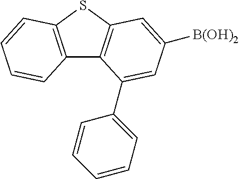 | 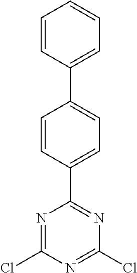 | 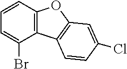 | 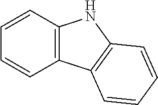 |
| 1-137 | 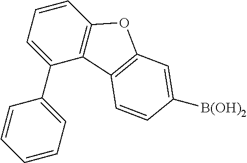 | 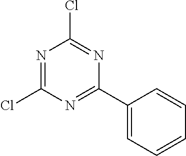 | 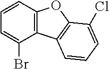 | 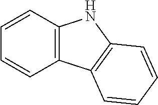 |
| 1-144 | 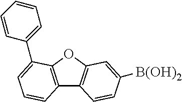 | 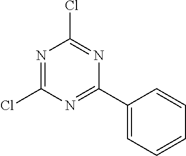 | 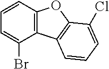 | 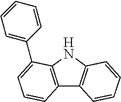 |
| 1-148 | 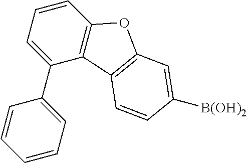 | 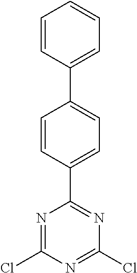 | 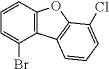 | 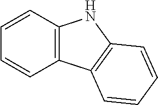 |
| 1-154 | 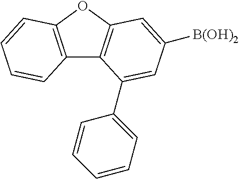 | 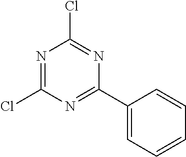 | 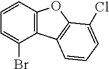 | 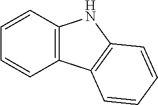 |
| 1-158 | 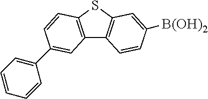 | 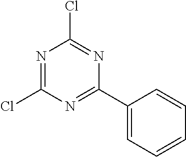 | 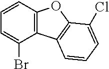 | 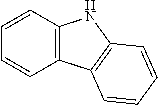 |
| 1-161 | 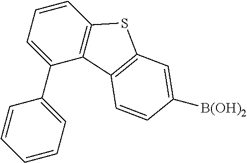 | 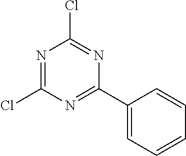 | 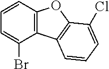 | 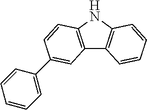 |
| 1-166 | 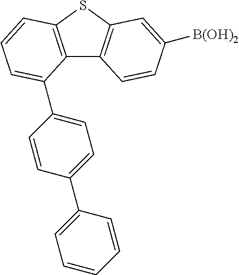 | 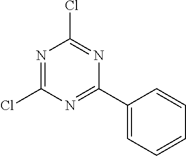 | 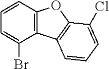 | 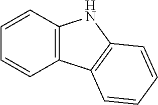 |
| 1-176 | 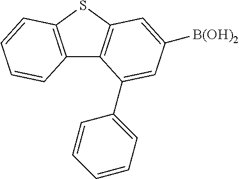 | 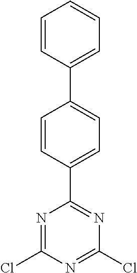 | 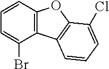 | 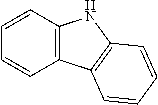 |
| 1-193 | 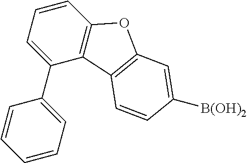 | 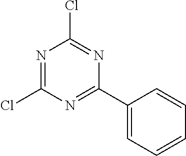 | 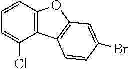 | 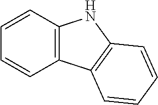 |
| 1-195 | 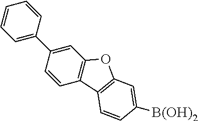 | 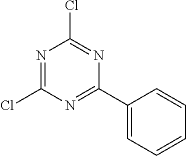 | 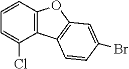 | 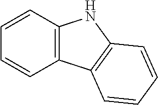 |
| 1-197 | 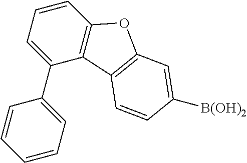 | 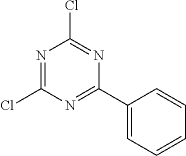 | 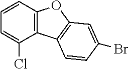 | 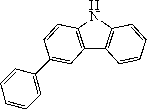 |
| 1-198 | 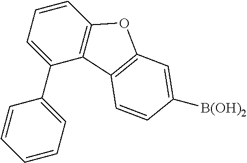 | 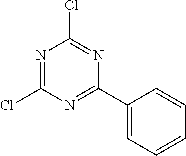 | 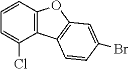 | 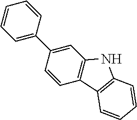 |
| 1-201 | 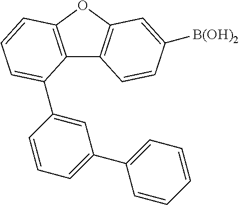 | 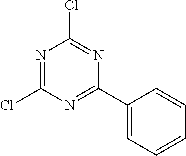 | 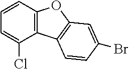 | 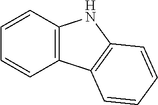 |
| 1-205 | 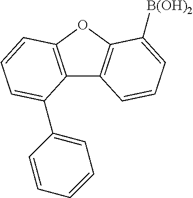 | 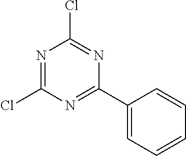 | 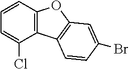 | 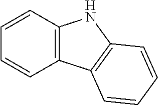 |
| 1-208 | 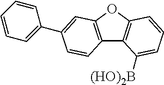 | 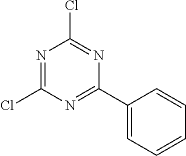 | 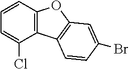 | 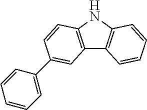 |
| 1-209 | 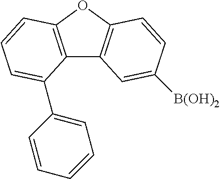 | 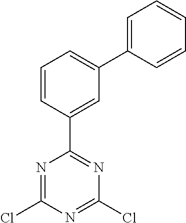 | 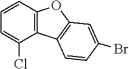 | 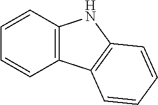 |
| 1-210 | 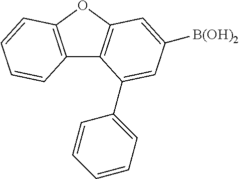 | 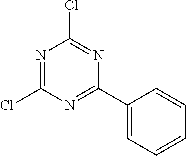 | 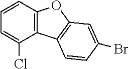 | 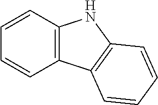 |
| 1-213 | 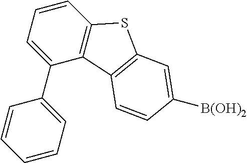 | 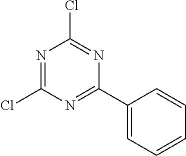 | 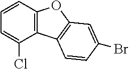 | 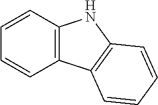 |
| 1-216 | 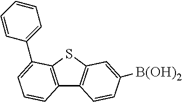 | 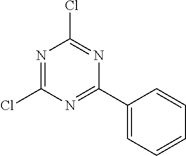 | 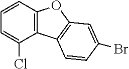 | 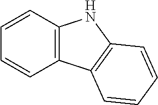 |
| 1-217 | 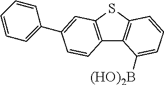 | 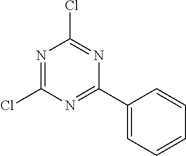 | 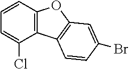 | 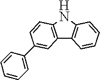 |
| 1-219 | 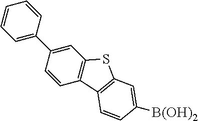 | 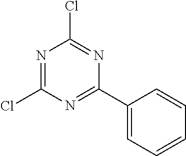 | 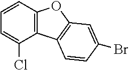 | 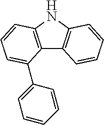 |
| 1-222 | 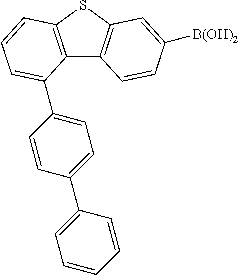 | 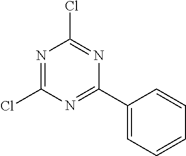 | 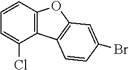 | 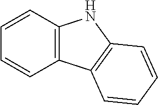 |
| 1-224 | 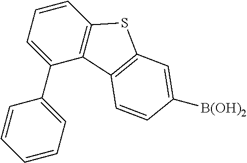 | 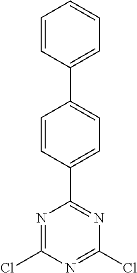 | 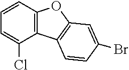 | 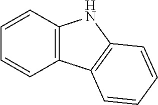 |
| 1-226 | 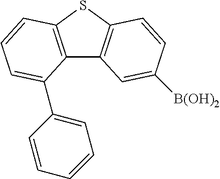 | 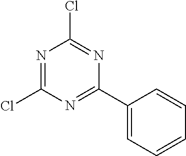 | 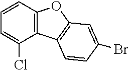 | 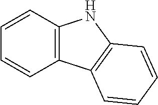 |
| 1-227 | 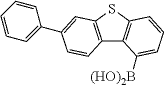 | 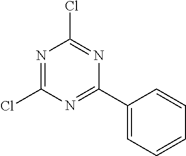 | 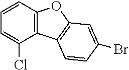 | 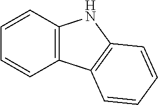 |
| 1-232 | 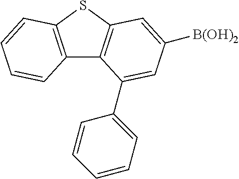 | 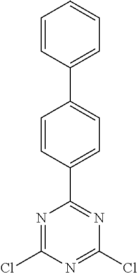 | 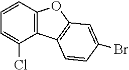 | 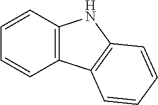 |
| 1-249 | 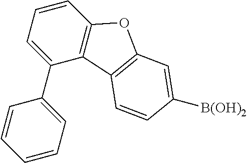 | 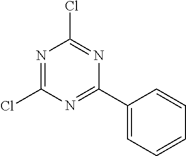 | 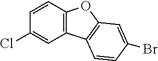 | 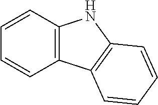 |
| 1-251 | 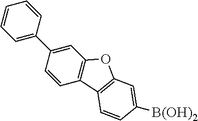 | 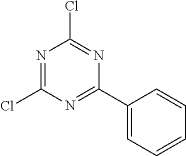 | 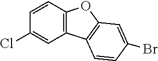 | 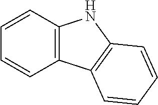 |
| 1-253 | 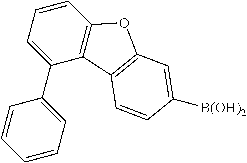 | 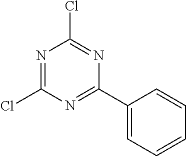 | 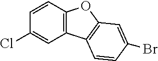 | 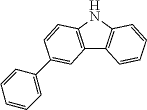 |
| 1-256 | 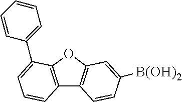 | 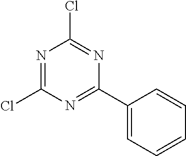 | 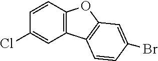 | 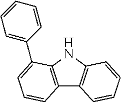 |
| 1-257 | 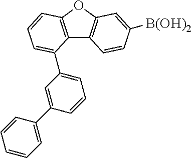 | 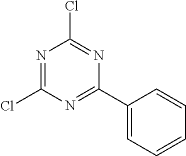 | 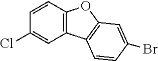 | 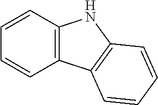 |
| 1-260 | 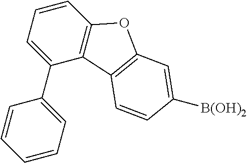 | 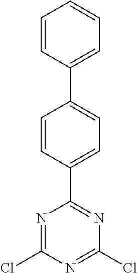 | 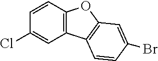 | 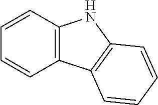 |
| 1-263 | 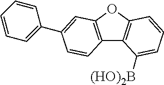 | 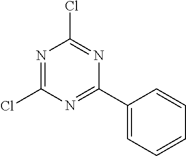 | 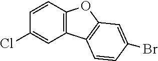 | 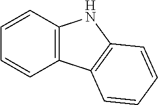 |
| 1-266 | 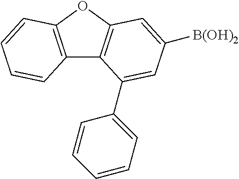 | 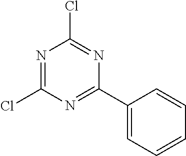 | 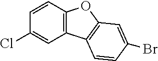 | 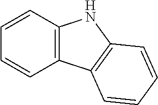 |
| 1-270 | 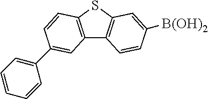 | 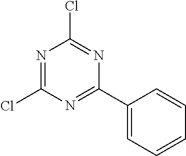 | 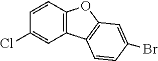 | 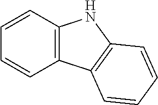 |
| 1-273 | 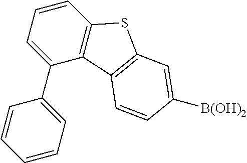 | 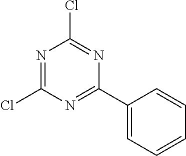 | 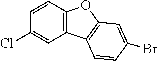 | 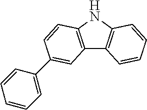 |
| 1-274 | 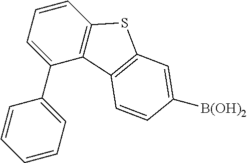 | 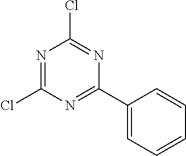 | 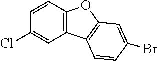 | 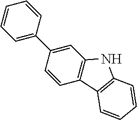 |
| 1-276 | 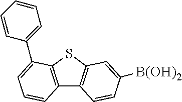 | 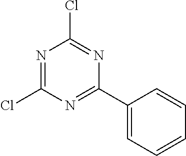 | 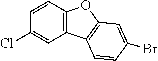 | 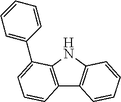 |
| 1-279 | 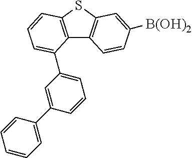 | 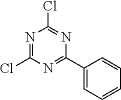 | 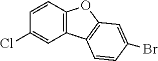 | 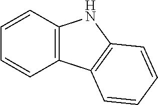 |
| 1-280 | 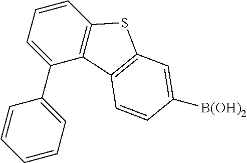 | 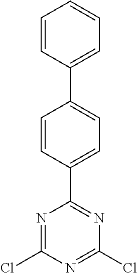 | 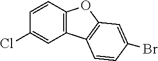 | 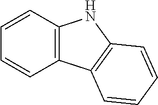 |
| 1-287 | 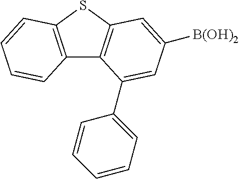 | 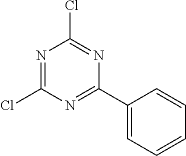 | 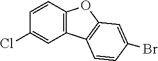 | 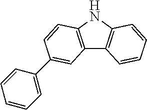 |
| 1-301 | 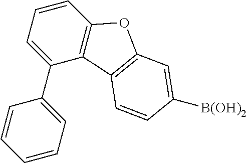 | 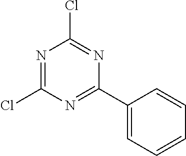 | 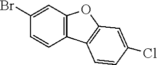 | 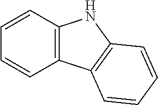 |
| 1-302 | 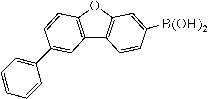 | 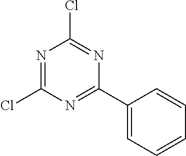 | 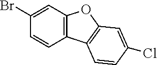 | 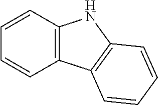 |
| 1-305 | 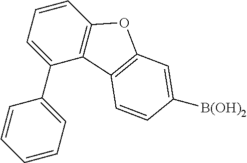 | 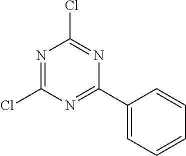 | 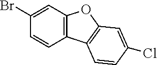 | 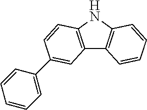 |
| 1-310 | 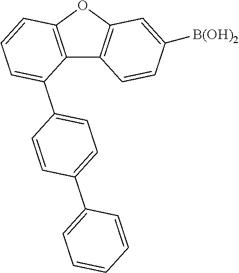 | 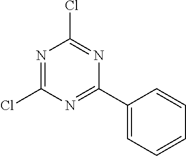 | 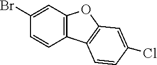 | 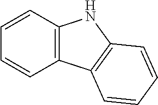 |
| 1-312 | 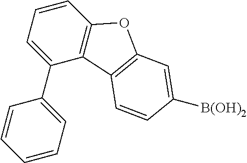 | 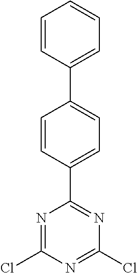 | 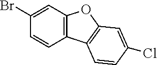 | 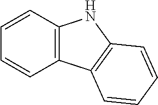 |
| 1-313 | 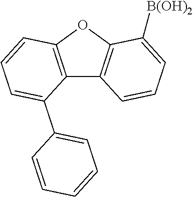 | 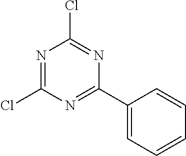 | 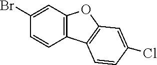 | 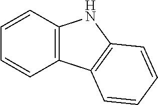 |
| 1-316 | 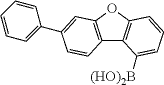 | 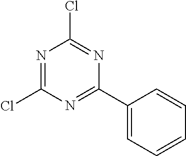 | 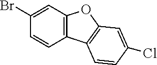 | 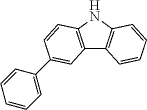 |
| 1-320 | 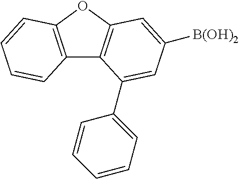 | 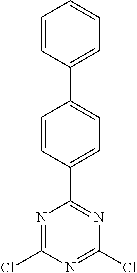 | 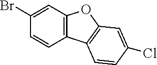 | 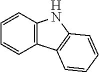 |
| 1-321 | 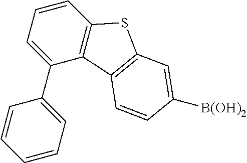 | 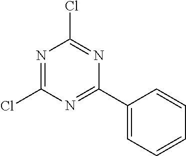 | 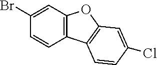 | 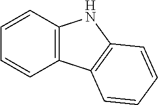 |
| 1-324 | 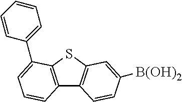 | 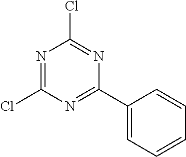 | 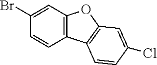 | 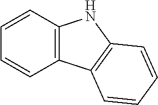 |
| 1-326 | 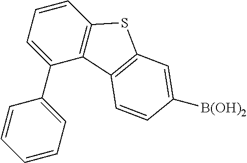 | 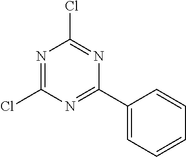 | 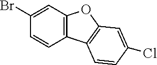 | 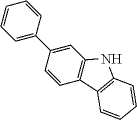 |
| 1-330 | 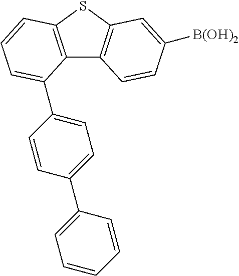 | 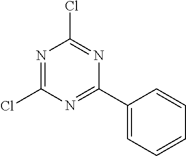 | 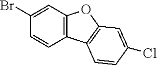 | 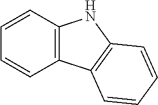 |
| 1-333 | 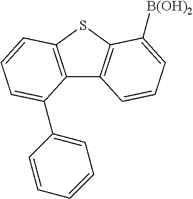 | 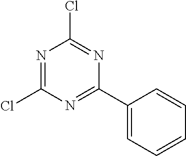 | 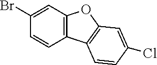 | 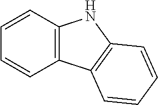 |
| 1-337 | 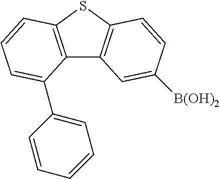 | 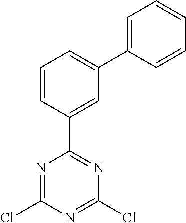 | 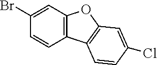 | 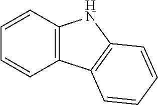 |
| 1-355 | 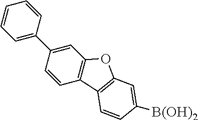 | 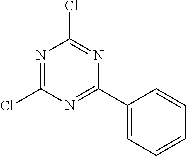 | 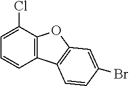 | 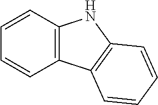 |
| 1-356 | 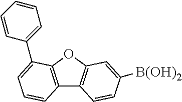 | 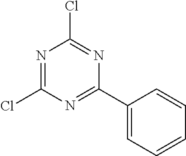 | 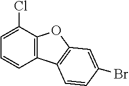 | 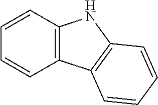 |
| 1-360 | 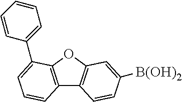 | 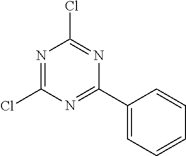 | 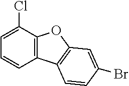 | 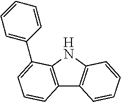 |
| 1-364 | 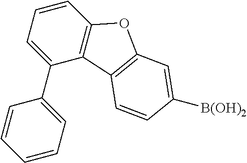 | 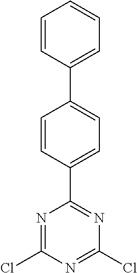 | 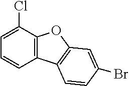 | 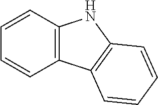 |
| 1-368 | 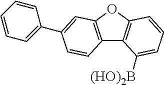 | 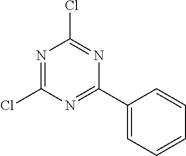 | 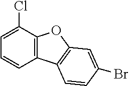 | 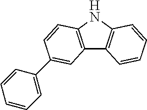 |
| 1-372 | 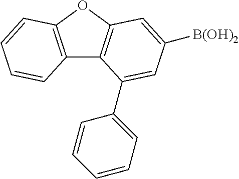 | 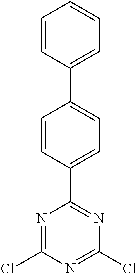 | 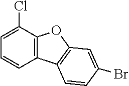 | 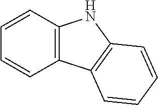 |
| 1-373 | 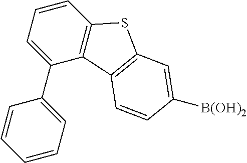 | 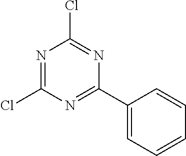 | 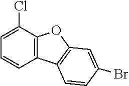 | 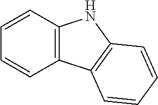 |
| 1-379 | 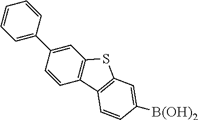 | 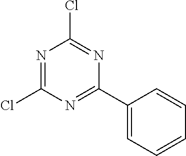 | 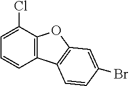 | 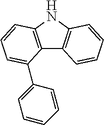 |
| 1-381 | 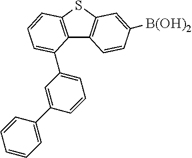 | 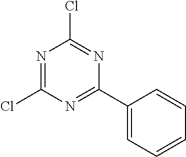 | 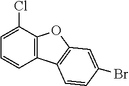 | 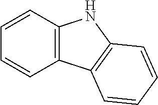 |
| 1-388 | 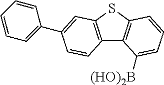 | 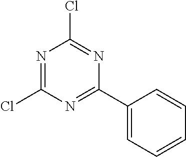 | 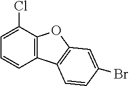 | 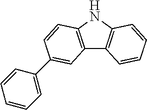 |

| Compound | Target Compound | Yield |
|---|---|---|
| 1-1 | 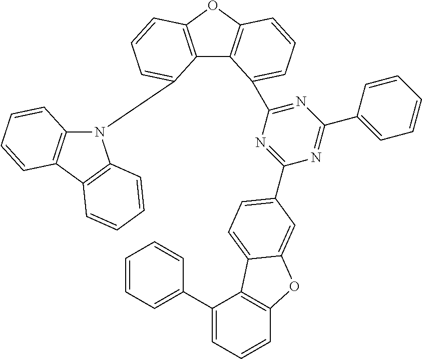 [SMILES: c1ccc(-c2nc(-c3ccc4c(c3)oc3cccc(-c5ccccc5)c34)nc(-c3cccc4oc5cccc(-n6c7ccccc7c7ccccc76)c5c34)n2)cc1] | 61% |
| 1-9 | 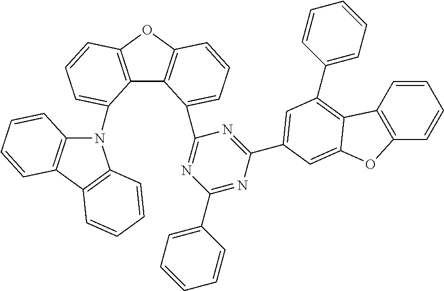 [SMILES: c1ccc(-c2nc(-c3cc(-c4ccccc4)c4c(c3)oc3ccccc34)nc(-c3cccc4oc5cccc(-n6c7ccccc7c7ccccc76)c5c34)n2)cc1] | 59% |
| 1-12 | 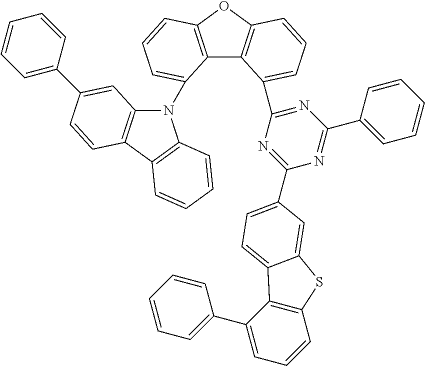 [SMILES: c1ccc(-c2ccc3c4ccccc4n(-c4cccc5oc6cccc(-c7nc(-c8ccccc8)nc(-c8ccc9c(c8)sc8cccc(-c%10ccccc%10)c89)n7)c6c45)c3c2)cc1] | 53% |
| 1-25 | 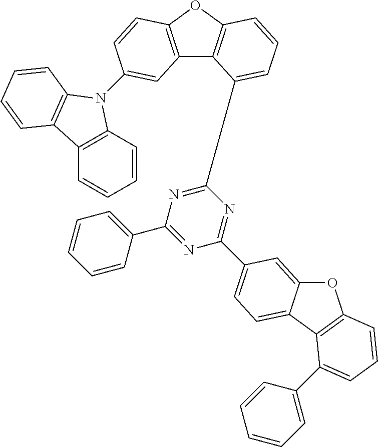 [SMILES: c1ccc(-c2nc(-c3ccc4c(c3)oc3cccc(-c5ccccc5)c34)nc(-c3cccc4oc5ccc(-n6c7ccccc7c7ccccc76)cc5c34)n2)cc1] | 59% |
| 1-28 | 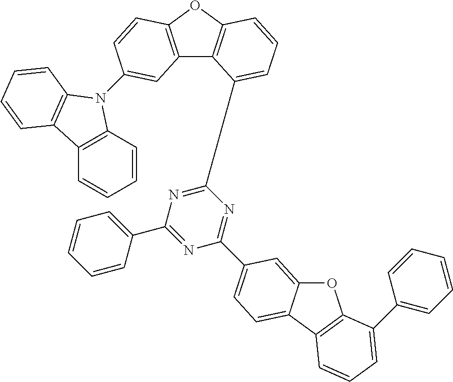 [SMILES: c1ccc(-c2nc(-c3ccc4c(c3)oc3c(-c5ccccc5)cccc34)nc(-c3cccc4oc5ccc(-n6c7ccccc7c7ccccc76)cc5c34)n2)cc1] | 63% |
| 1-30 | 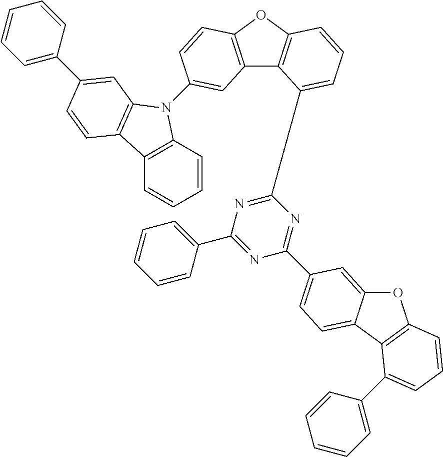 [SMILES: c1ccc(-c2ccc3c4ccccc4n(-c4ccc5oc6cccc(-c7nc(-c8ccccc8)nc(-c8ccc9c(c8)oc8cccc(-c%10ccccc%10)c89)n7)c6c5c4)c3c2)cc1] | 60% |
| 1-34 | 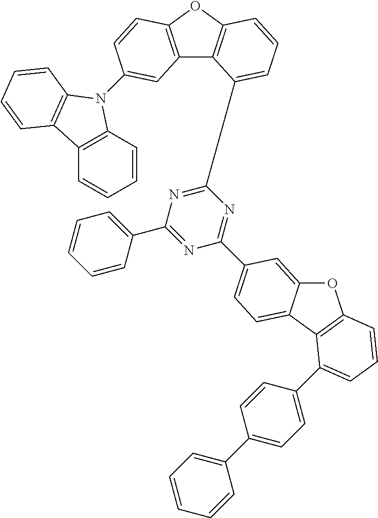 [SMILES: c1ccc(-c2ccc(-c3cccc4oc5cc(-c6nc(-c7ccccc7)nc(-c7cccc8oc9ccc(-n%10c%11ccccc%11c%11ccccc%11%10)cc9c78)n6)ccc5c34)cc2)cc1] | 59% |
| 1-36 | 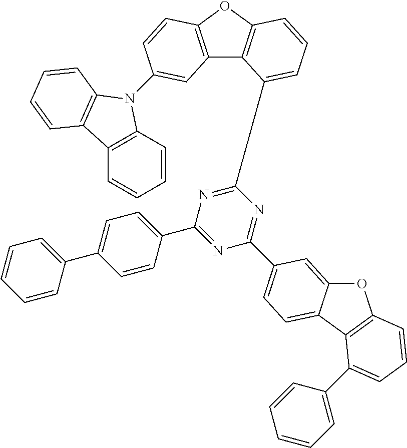 [SMILES: c1ccc(-c2ccc(-c3nc(-c4ccc5c(c4)oc4cccc(-c6ccccc6)c45)nc(-c4cccc5oc6ccc(-n7c8ccccc8c8ccccc87)cc6c45)n3)cc2)cc1] | 82% |
| 1-39 | 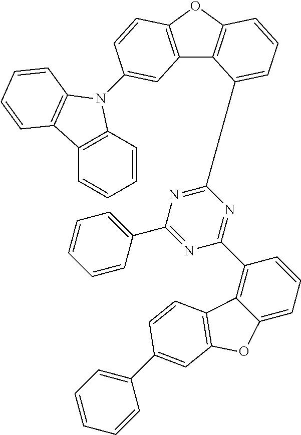 [SMILES: c1ccc(-c2ccc3c(c2)oc2cccc(-c4nc(-c5ccccc5)nc(-c5cccc6oc7ccc(-n8c9ccccc9c9ccccc98)cc7c56)n4)c23)cc1] | 77% |
| 1-42 | 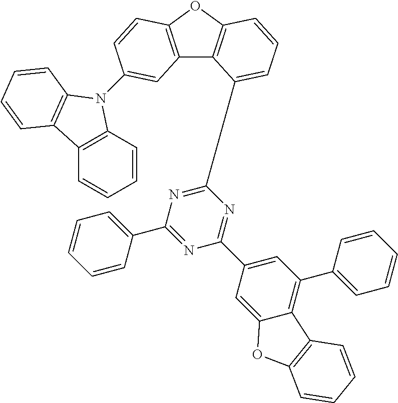 [SMILES: c1ccc(-c2nc(-c3cc(-c4ccccc4)c4c(c3)oc3ccccc34)nc(-c3cccc4oc5ccc(-n6c7ccccc7c7ccccc76)cc5c34)n2)cc1] | 65% |
| 1-46 | 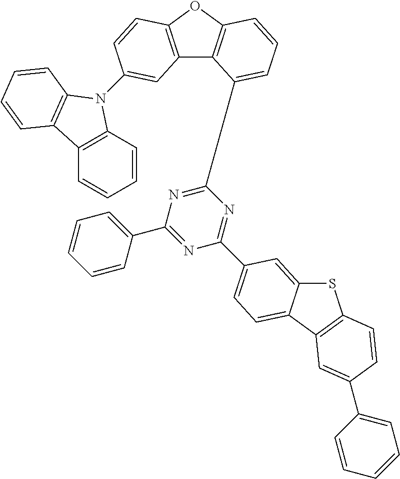 [SMILES: c1ccc(-c2ccc3sc4cc(-c5nc(-c6ccccc6)nc(-c6cccc7oc8ccc(-n9c%10ccccc%10c%10ccccc%109)cc8c67)n5)ccc4c3c2)cc1] | 61% |
| 1-50 | 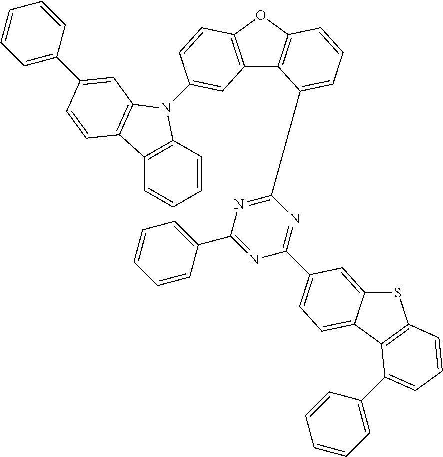 [SMILES: c1ccc(-c2ccc3c4ccccc4n(-c4ccc5oc6cccc(-c7nc(-c8ccccc8)nc(-c8ccc9c(c8)sc8cccc(-c%10ccccc%10)c89)n7)c6c5c4)c3c2)cc1] | 69% |
| 1-52 | 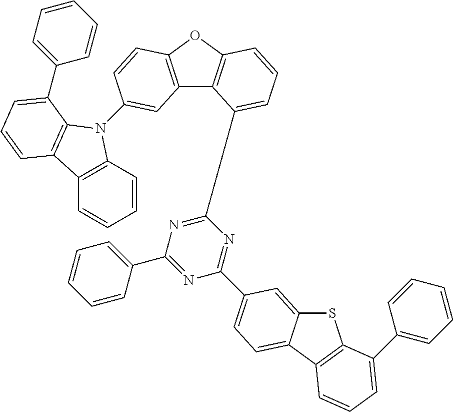 [SMILES: c1ccc(-c2nc(-c3ccc4c(c3)sc3c(-c5ccccc5)cccc34)nc(-c3cccc4oc5ccc(-n6c7ccccc7c7cccc(-c8ccccc8)c76)cc5c34)n2)cc1] | 83% |
| 1-56 | 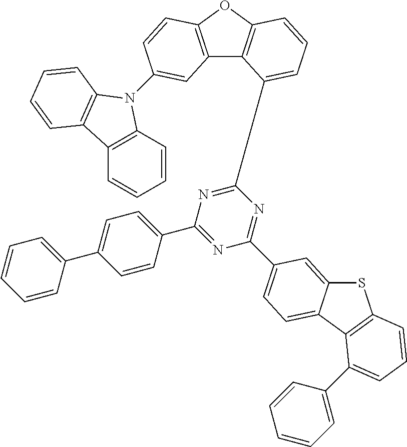 [SMILES: c1ccc(-c2ccc(-c3nc(-c4ccc5c(c4)sc4cccc(-c6ccccc6)c45)nc(-c4cccc5oc6ccc(-n7c8ccccc8c8ccccc87)cc6c45)n3)cc2)cc1] | 92% |
| 1-58 | 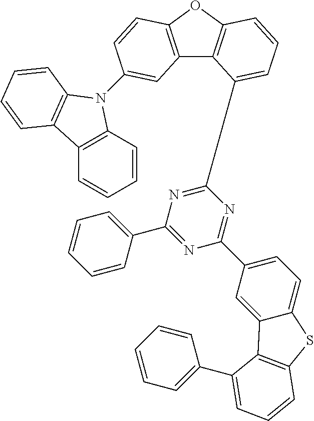 [SMILES: c1ccc(-c2nc(-c3ccc4sc5cccc(-c6ccccc6)c5c4c3)nc(-c3cccc4oc5ccc(-n6c7ccccc7c7ccccc76)cc5c34)n2)cc1] | 91% |
| 1-60 | 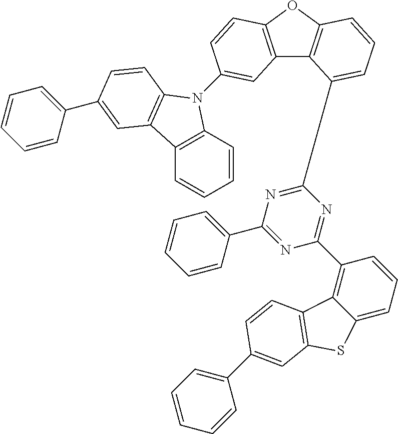 [SMILES: c1ccc(-c2ccc3c(c2)sc2cccc(-c4nc(-c5ccccc5)nc(-c5cccc6oc7ccc(-n8c9ccccc9c9cc(-c%10ccccc%10)ccc98)cc7c56)n4)c23)cc1] | 78% |
| 1-81 | 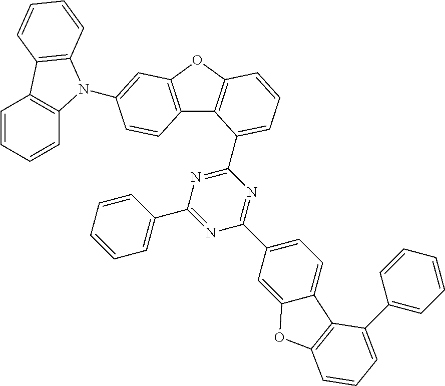 [SMILES: c1ccc(-c2nc(-c3ccc4c(c3)oc3cccc(-c5ccccc5)c34)nc(-c3cccc4oc5cc(-n6c7ccccc7c7ccccc76)ccc5c34)n2)cc1] | 93% |
| 1-85 | 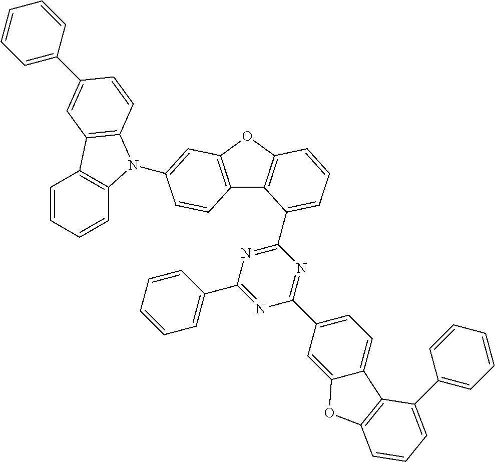 [SMILES: c1ccc(-c2ccc3c(c2)c2ccccc2n3-c2ccc3c(c2)oc2cccc(-c4nc(-c5ccccc5)nc(-c5ccc6c(c5)oc5cccc(-c7ccccc7)c56)n4)c23)cc1] | 74% |
| 1-87 | 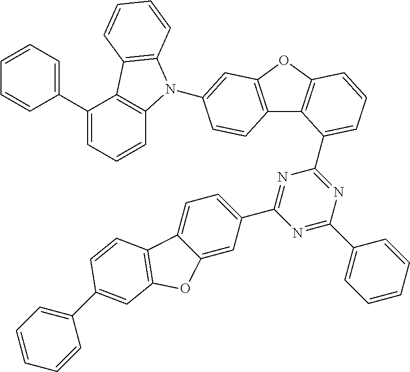 [SMILES: c1ccc(-c2ccc3c(c2)oc2cc(-c4nc(-c5ccccc5)nc(-c5cccc6oc7cc(-n8c9ccccc9c9c(-c%10ccccc%10)cccc98)ccc7c56)n4)ccc23)cc1] | 63% |
| 1-90 | 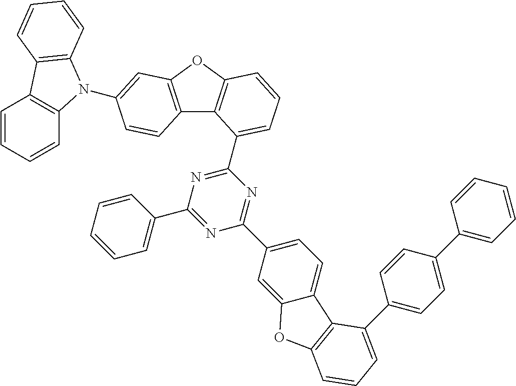 [SMILES: c1ccc(-c2ccc(-c3cccc4oc5cc(-c6nc(-c7ccccc7)nc(-c7cccc8oc9cc(-n%10c%11ccccc%11c%11ccccc%11%10)ccc9c78)n6)ccc5c34)cc2)cc1] | 65% |
| 1-92 | 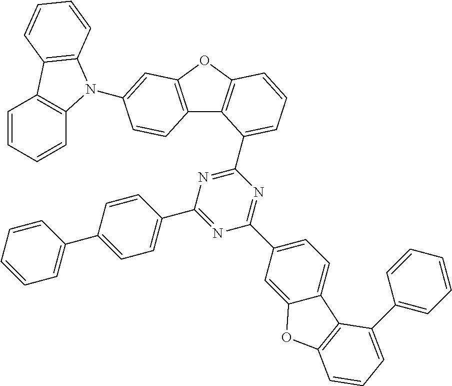 [SMILES: c1ccc(-c2ccc(-c3nc(-c4ccc5c(c4)oc4cccc(-c6ccccc6)c45)nc(-c4cccc5oc6cc(-n7c8ccccc8c8ccccc87)ccc6c45)n3)cc2)cc1] | 68% |
| 1-95 | 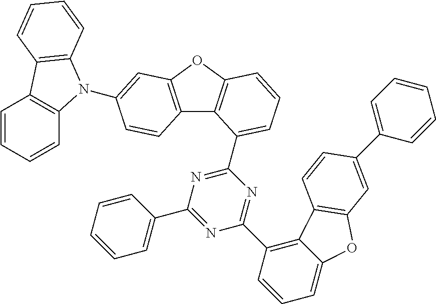 [SMILES: c1ccc(-c2ccc3c(c2)oc2cccc(-c4nc(-c5ccccc5)nc(-c5cccc6oc7cc(-n8c9ccccc9c9ccccc98)ccc7c56)n4)c23)cc1] | 79% |
| 1-98 | 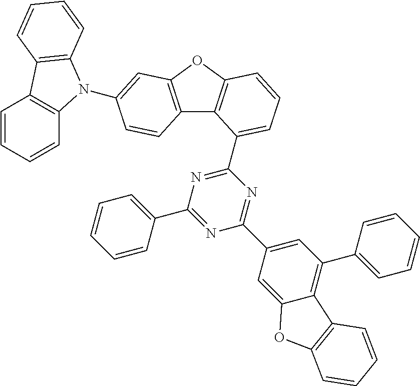 [SMILES: c1ccc(-c2nc(-c3cc(-c4ccccc4)c4c(c3)oc3ccccc34)nc(-c3cccc4oc5cc(-n6c7ccccc7c7ccccc76)ccc5c34)n2)cc1] | 77% |
| 1-100 | 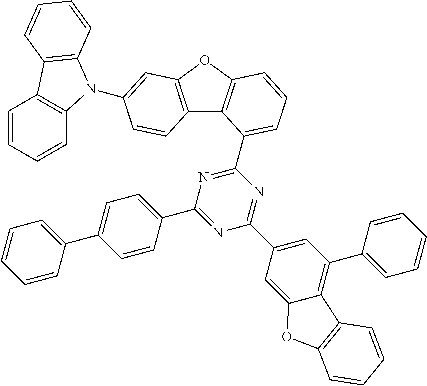 [SMILES: c1ccc(-c2ccc(-c3nc(-c4cc(-c5ccccc5)c5c(c4)oc4ccccc45)nc(-c4cccc5oc6cc(-n7c8ccccc8c8ccccc87)ccc6c45)n3)cc2)cc1] | 83% |
| 1-103 | 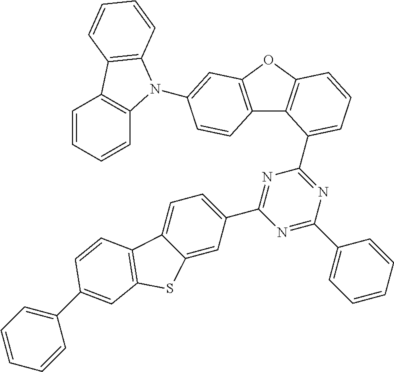 [SMILES: c1ccc(-c2ccc3c(c2)sc2cc(-c4nc(-c5ccccc5)nc(-c5cccc6oc7cc(-n8c9ccccc9c9ccccc98)ccc7c56)n4)ccc23)cc1] | 88% |
| 1-106 | 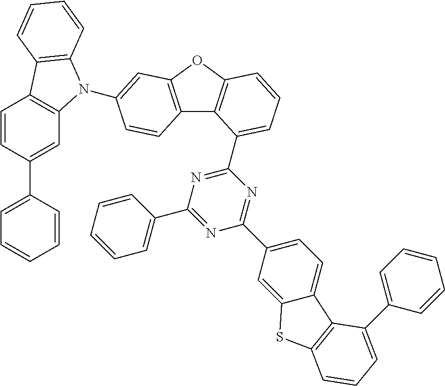 [SMILES: c1ccc(-c2ccc3c4ccccc4n(-c4ccc5c(c4)oc4cccc(-c6nc(-c7ccccc7)nc(-c7ccc8c(c7)sc7cccc(-c9ccccc9)c78)n6)c45)c3c2)cc1] | 76% |
| 1-109 | 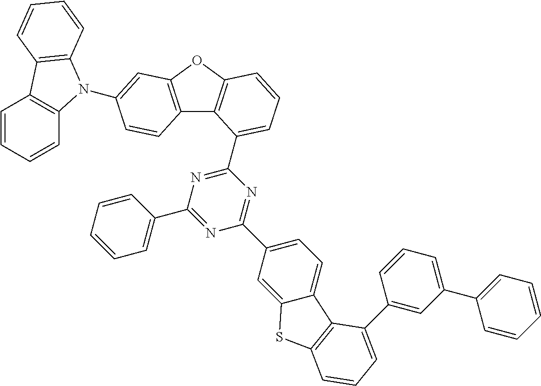 [SMILES: c1ccc(-c2cccc(-c3cccc4sc5cc(-c6nc(-c7ccccc7)nc(-c7cccc8oc9cc(-n%10c%11ccccc%11c%11ccccc%11%10)ccc9c78)n6)ccc5c34)c2)cc1] | 91% |
| 1-115 | 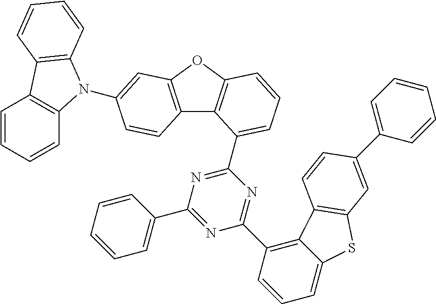 [SMILES: c1ccc(-c2ccc3c(c2)sc2cccc(-c4nc(-c5ccccc5)nc(-c5cccc6oc7cc(-n8c9ccccc9c9ccccc98)ccc7c56)n4)c23)cc1] | 71% |
| 1-120 | 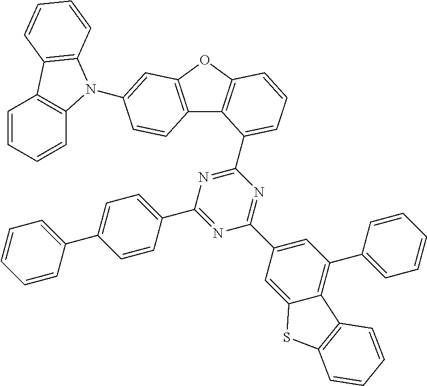 [SMILES: c1ccc(-c2ccc(-c3nc(-c4cc(-c5ccccc5)c5c(c4)sc4ccccc45)nc(-c4cccc5oc6cc(-n7c8ccccc8c8ccccc87)ccc6c45)n3)cc2)cc1] | 79% |
| 1-137 | 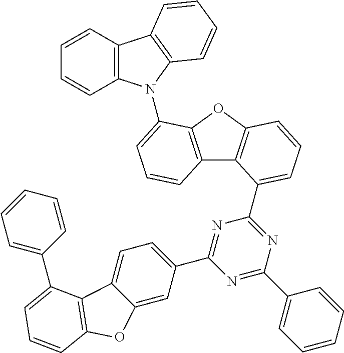 [SMILES: c1ccc(-c2nc(-c3ccc4c(c3)oc3cccc(-c5ccccc5)c34)nc(-c3cccc4oc5c(-n6c7ccccc7c7ccccc76)cccc5c34)n2)cc1] | 59% |
| 1-144 | 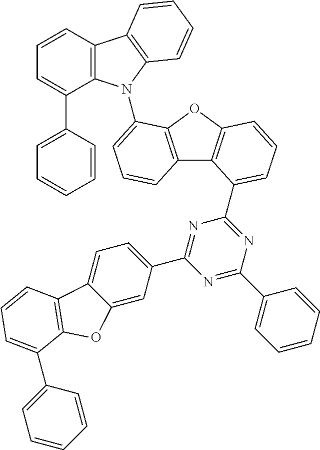 [SMILES: c1ccc(-c2nc(-c3ccc4c(c3)oc3c(-c5ccccc5)cccc34)nc(-c3cccc4oc5c(-n6c7ccccc7c7cccc(-c8ccccc8)c76)cccc5c34)n2)cc1] | 58% |
| 1-148 | 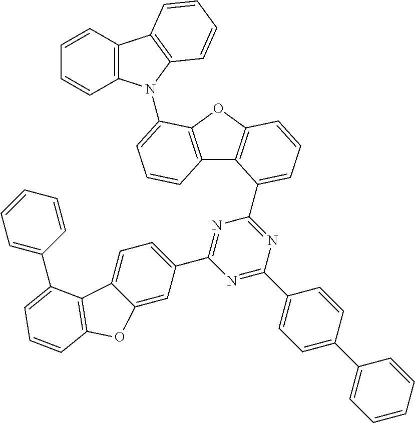 [SMILES: c1ccc(-c2ccc(-c3nc(-c4ccc5c(c4)oc4cccc(-c6ccccc6)c45)nc(-c4cccc5oc6c(-n7c8ccccc8c8ccccc87)cccc6c45)n3)cc2)cc1] | 69% |
| 1-154 | 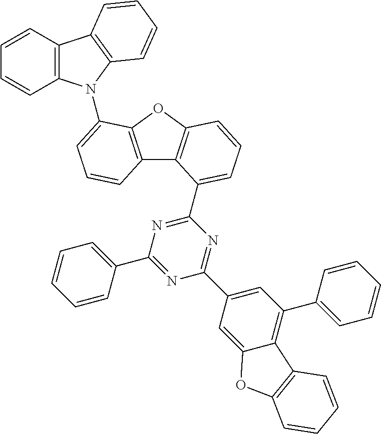 [SMILES: c1ccc(-c2nc(-c3cc(-c4ccccc4)c4c(c3)oc3ccccc34)nc(-c3cccc4oc5c(-n6c7ccccc7c7ccccc76)cccc5c34)n2)cc1] | 74% |
| 1-158 | 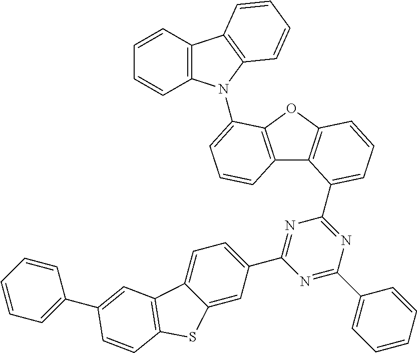 [SMILES: c1ccc(-c2ccc3sc4cc(-c5nc(-c6ccccc6)nc(-c6cccc7oc8c(-n9c%10ccccc%10c%10ccccc%109)cccc8c67)n5)ccc4c3c2)cc1] | 66% |
| 1-161 | 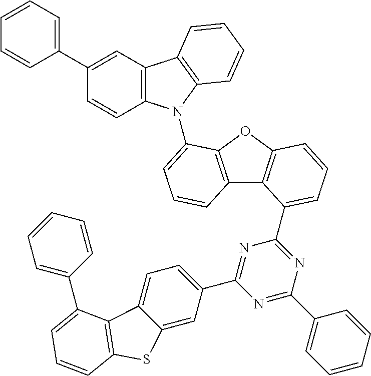 [SMILES: c1ccc(-c2ccc3c(c2)c2ccccc2n3-c2cccc3c2oc2cccc(-c4nc(-c5ccccc5)nc(-c5ccc6c(c5)sc5cccc(-c7ccccc7)c56)n4)c23)cc1] | 58% |
| 1-166 | 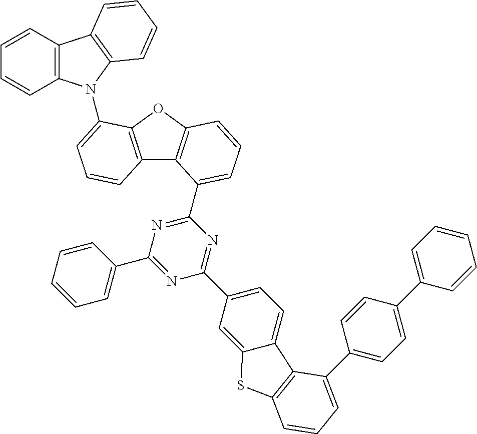 [SMILES: c1ccc(-c2ccc(-c3cccc4sc5cc(-c6nc(-c7ccccc7)nc(-c7cccc8oc9c(-n%10c%11ccccc%11c%11ccccc%11%10)cccc9c78)n6)ccc5c34)cc2)cc1] | 45% |
| 1-176 | 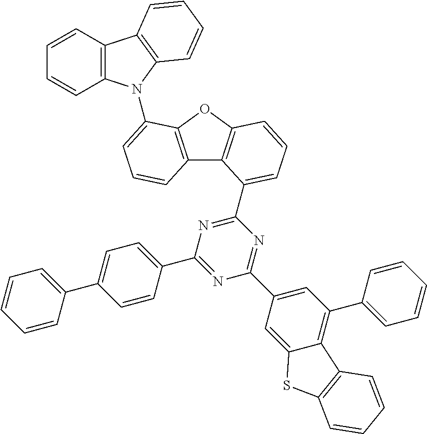 [SMILES: c1ccc(-c2ccc(-c3nc(-c4cc(-c5ccccc5)c5c(c4)sc4ccccc45)nc(-c4cccc5oc6c(-n7c8ccccc8c8ccccc87)cccc6c45)n3)cc2)cc1] | 65% |
| 1-193 | 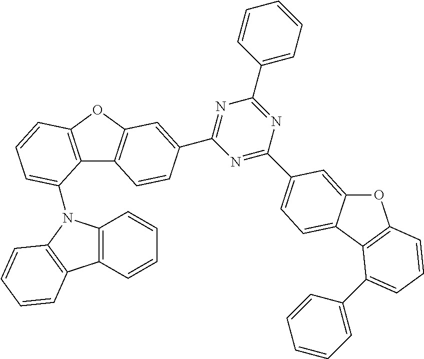 [SMILES: c1ccc(-c2nc(-c3ccc4c(c3)oc3cccc(-c5ccccc5)c34)nc(-c3ccc4c(c3)oc3cccc(-n5c6ccccc6c6ccccc65)c34)n2)cc1] | 72% |
| 1-195 | 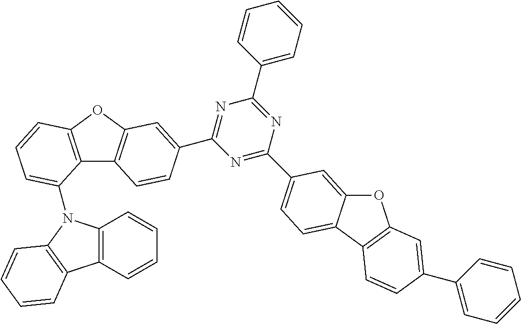 [SMILES: c1ccc(-c2ccc3c(c2)oc2cc(-c4nc(-c5ccccc5)nc(-c5ccc6c(c5)oc5cccc(-n7c8ccccc8c8ccccc87)c56)n4)ccc23)cc1] | 61% |
| 1-197 | 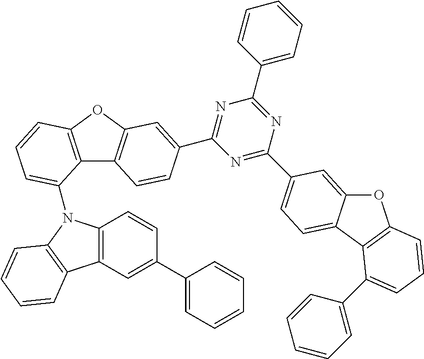 [SMILES: c1ccc(-c2ccc3c(c2)c2ccccc2n3-c2cccc3oc4cc(-c5nc(-c6ccccc6)nc(-c6ccc7c(c6)oc6cccc(-c8ccccc8)c67)n5)ccc4c23)cc1] | 92% |
| 1-198 | 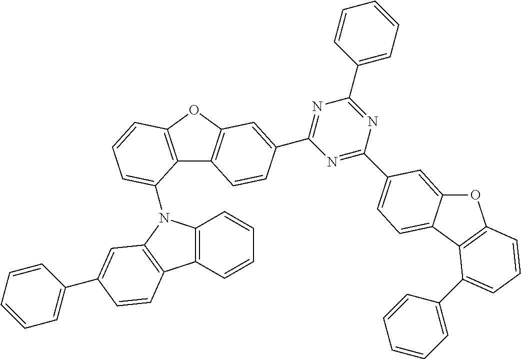 [SMILES: c1ccc(-c2ccc3c4ccccc4n(-c4cccc5oc6cc(-c7nc(-c8ccccc8)nc(-c8ccc9c(c8)oc8cccc(-c%10ccccc%10)c89)n7)ccc6c45)c3c2)cc1] | 97% |
| 1-201 | 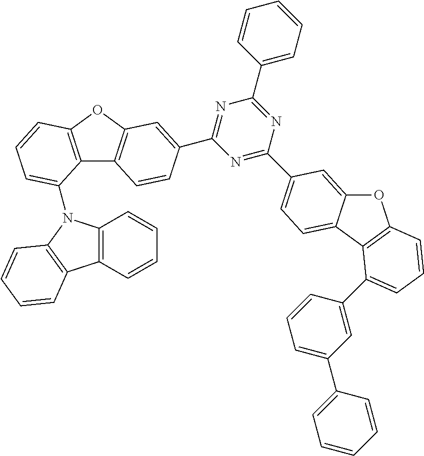 [SMILES: c1ccc(-c2cccc(-c3cccc4oc5cc(-c6nc(-c7ccccc7)nc(-c7ccc8c(c7)oc7cccc(-n9c%10ccccc%10c%10ccccc%109)c78)n6)ccc5c34)c2)cc1] | 98% |
| 1-205 | 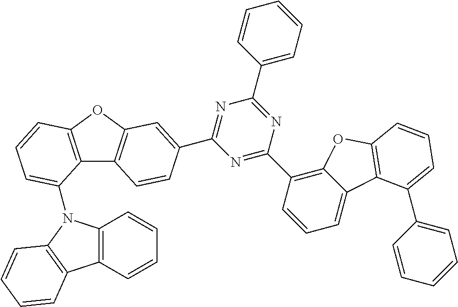 [SMILES: c1ccc(-c2nc(-c3ccc4c(c3)oc3cccc(-n5c6ccccc6c6ccccc65)c34)nc(-c3cccc4c3oc3cccc(-c5ccccc5)c34)n2)cc1] | 92% |
| 1-208 | 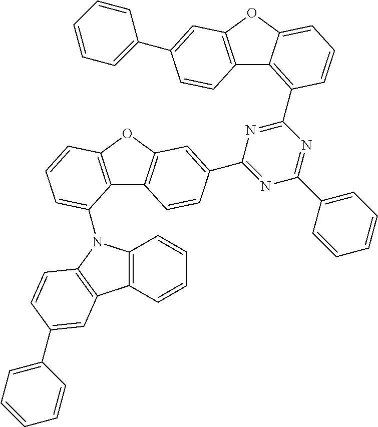 [SMILES: c1ccc(-c2ccc3c(c2)oc2cccc(-c4nc(-c5ccccc5)nc(-c5ccc6c(c5)oc5cccc(-n7c8ccccc8c8cc(-c9ccccc9)ccc87)c56)n4)c23)cc1] | 76% |
| 1-209 | 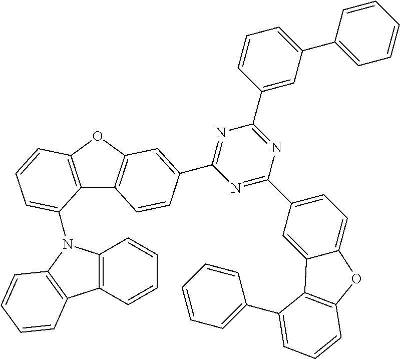 [SMILES: c1ccc(-c2cccc(-c3nc(-c4ccc5c(c4)oc4cccc(-n6c7ccccc7c7ccccc76)c45)nc(-c4ccc5oc6cccc(-c7ccccc7)c6c5c4)n3)c2)cc1] | 74% |
| 1-210 | 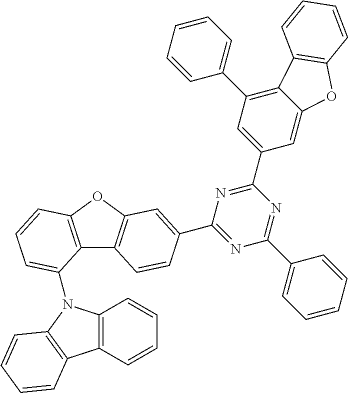 [SMILES: c1ccc(-c2nc(-c3ccc4c(c3)oc3cccc(-n5c6ccccc6c6ccccc65)c34)nc(-c3cc(-c4ccccc4)c4c(c3)oc3ccccc34)n2)cc1] | 79% |
| 1-213 | 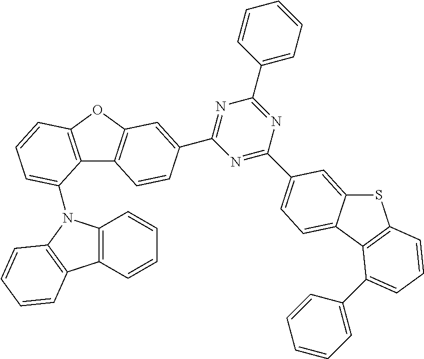 [SMILES: c1ccc(-c2nc(-c3ccc4c(c3)oc3cccc(-n5c6ccccc6c6ccccc65)c34)nc(-c3ccc4c(c3)sc3cccc(-c5ccccc5)c34)n2)cc1] | 88% |
| 1-216 | 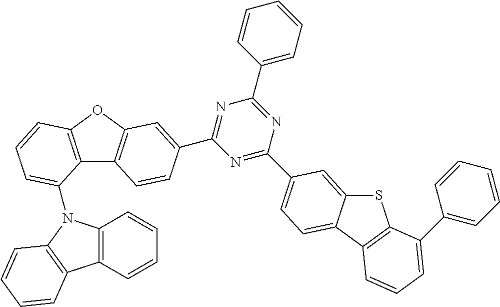 [SMILES: c1ccc(-c2nc(-c3ccc4c(c3)oc3cccc(-n5c6ccccc6c6ccccc65)c34)nc(-c3ccc4c(c3)sc3c(-c5ccccc5)cccc34)n2)cc1] | 83% |
| 1-217 | 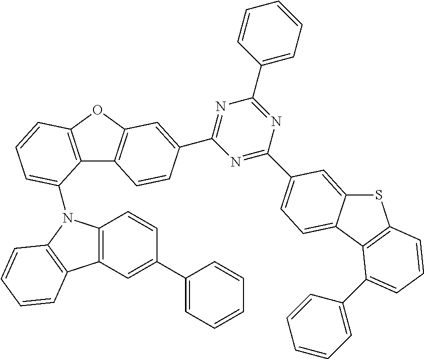 [SMILES: c1ccc(-c2ccc3c(c2)c2ccccc2n3-c2cccc3oc4cc(-c5nc(-c6ccccc6)nc(-c6ccc7c(c6)sc6cccc(-c8ccccc8)c67)n5)ccc4c23)cc1] | 81% |
| 1-219 | 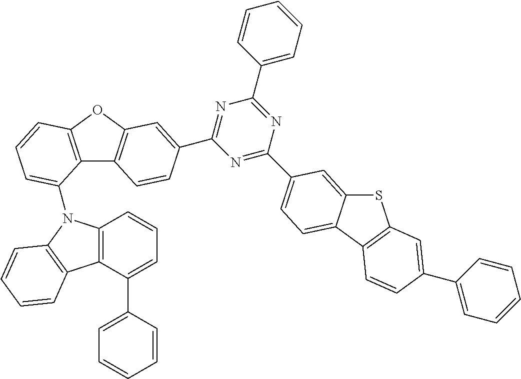 [SMILES: c1ccc(-c2ccc3c(c2)sc2cc(-c4nc(-c5ccccc5)nc(-c5ccc6c(c5)oc5cccc(-n7c8ccccc8c8c(-c9ccccc9)cccc87)c56)n4)ccc23)cc1] | 74% |
| 1-222 | 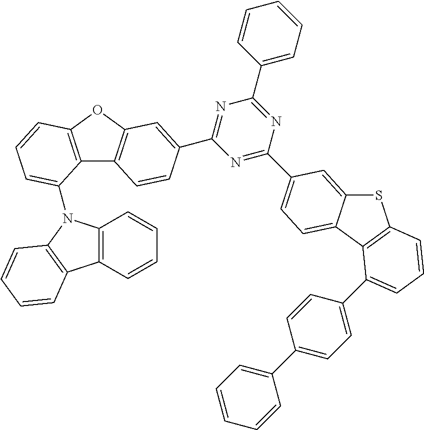 [SMILES: c1ccc(-c2ccc(-c3cccc4sc5cc(-c6nc(-c7ccccc7)nc(-c7ccc8c(c7)oc7cccc(-n9c%10ccccc%10c%10ccccc%109)c78)n6)ccc5c34)cc2)cc1] | 63% |
| 1-224 | 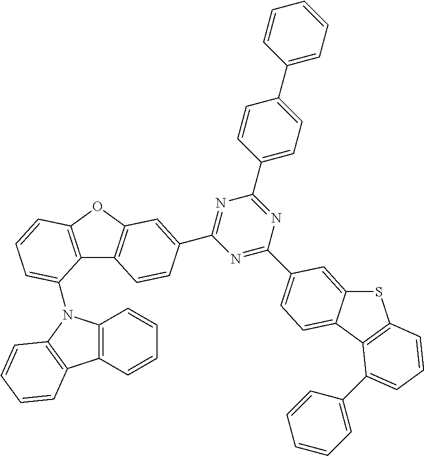 [SMILES: c1ccc(-c2ccc(-c3nc(-c4ccc5c(c4)oc4cccc(-n6c7ccccc7c7ccccc76)c45)nc(-c4ccc5c(c4)sc4cccc(-c6ccccc6)c45)n3)cc2)cc1] | 69% |
| 1-226 | 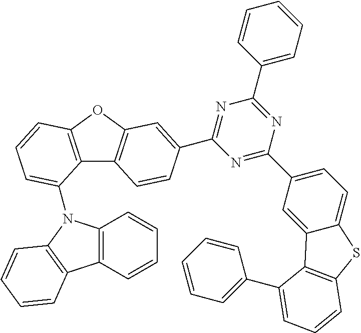 [SMILES: c1ccc(-c2nc(-c3ccc4c(c3)oc3cccc(-n5c6ccccc6c6ccccc65)c34)nc(-c3ccc4sc5cccc(-c6ccccc6)c5c4c3)n2)cc1] | 88% |
| 1-227 | 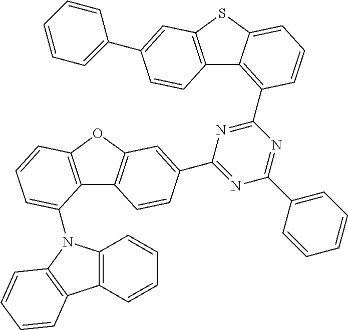 [SMILES: c1ccc(-c2ccc3c(c2)sc2cccc(-c4nc(-c5ccccc5)nc(-c5ccc6c(c5)oc5cccc(-n7c8ccccc8c8ccccc87)c56)n4)c23)cc1] | 94% |
| 1-232 | 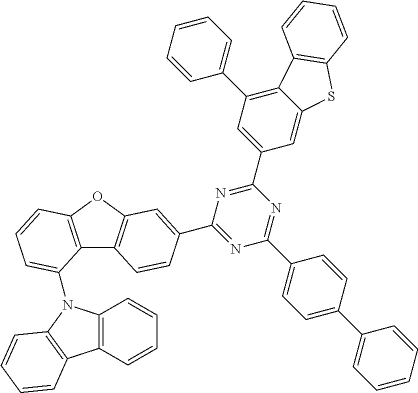 [SMILES: c1ccc(-c2ccc(-c3nc(-c4ccc5c(c4)oc4cccc(-n6c7ccccc7c7ccccc76)c45)nc(-c4cc(-c5ccccc5)c5c(c4)sc4ccccc45)n3)cc2)cc1] | 90% |
| 1-249 | 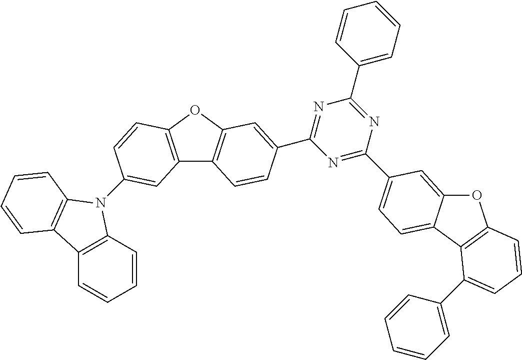 [SMILES: c1ccc(-c2nc(-c3ccc4c(c3)oc3ccc(-n5c6ccccc6c6ccccc65)cc34)nc(-c3ccc4c(c3)oc3cccc(-c5ccccc5)c34)n2)cc1] | 81% |
| 1-251 | 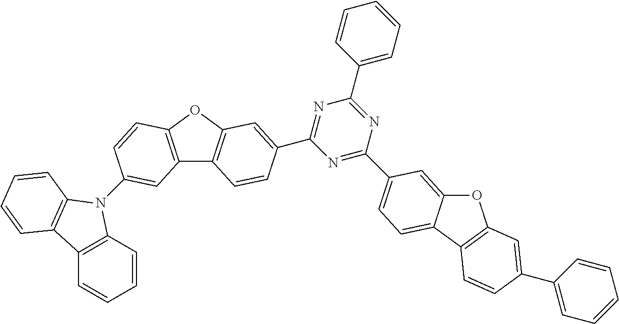 [SMILES: c1ccc(-c2ccc3c(c2)oc2cc(-c4nc(-c5ccccc5)nc(-c5ccc6c(c5)oc5ccc(-n7c8ccccc8c8ccccc87)cc56)n4)ccc23)cc1] | 84% |
| 1-253 | 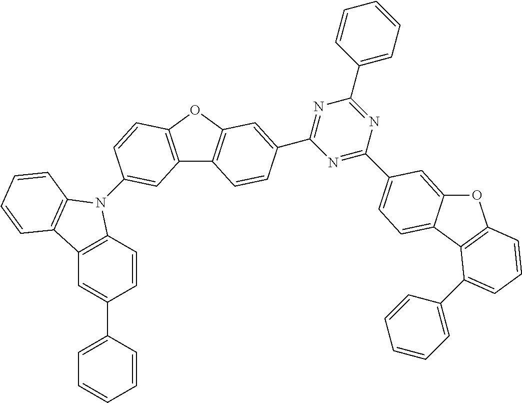 [SMILES: c1ccc(-c2ccc3c(c2)c2ccccc2n3-c2ccc3oc4cc(-c5nc(-c6ccccc6)nc(-c6ccc7c(c6)oc6cccc(-c8ccccc8)c67)n5)ccc4c3c2)cc1] | 86% |
| 1-256 | 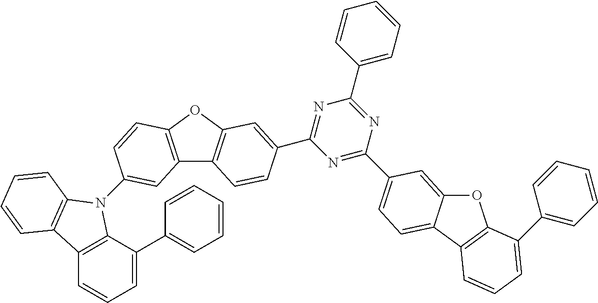 [SMILES: c1ccc(-c2nc(-c3ccc4c(c3)oc3ccc(-n5c6ccccc6c6cccc(-c7ccccc7)c65)cc34)nc(-c3ccc4c(c3)oc3c(-c5ccccc5)cccc34)n2)cc1] | 88% |
| 1-257 | 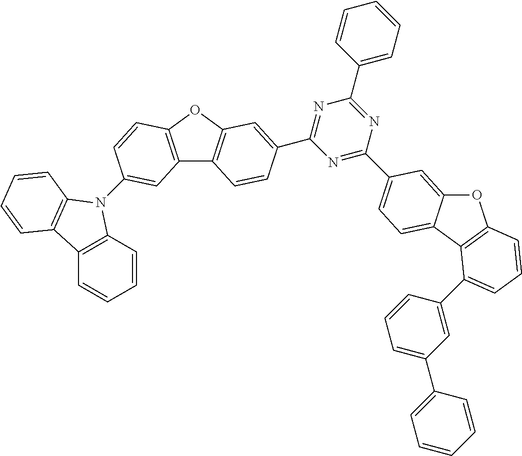 [SMILES: c1ccc(-c2cccc(-c3cccc4oc5cc(-c6nc(-c7ccccc7)nc(-c7ccc8c(c7)oc7ccc(-n9c%10ccccc%10c%10ccccc%109)cc78)n6)ccc5c34)c2)cc1] | 90% |
| 1-260 | 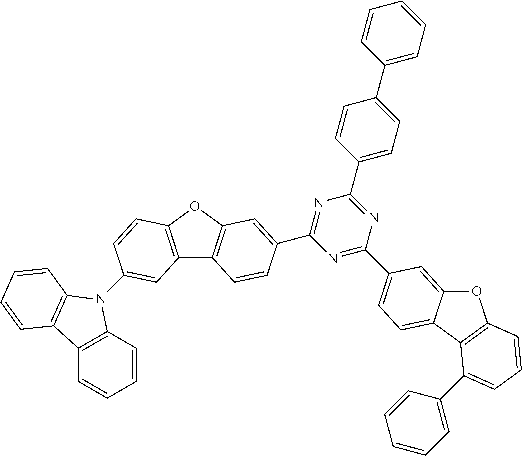 [SMILES: c1ccc(-c2ccc(-c3nc(-c4ccc5c(c4)oc4ccc(-n6c7ccccc7c7ccccc76)cc45)nc(-c4ccc5c(c4)oc4cccc(-c6ccccc6)c45)n3)cc2)cc1] | 94% |
| 1-263 | 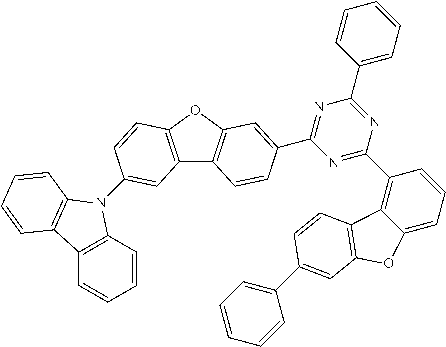 [SMILES: c1ccc(-c2ccc3c(c2)oc2cccc(-c4nc(-c5ccccc5)nc(-c5ccc6c(c5)oc5ccc(-n7c8ccccc8c8ccccc87)cc56)n4)c23)cc1] | 93% |
| 1-266 | 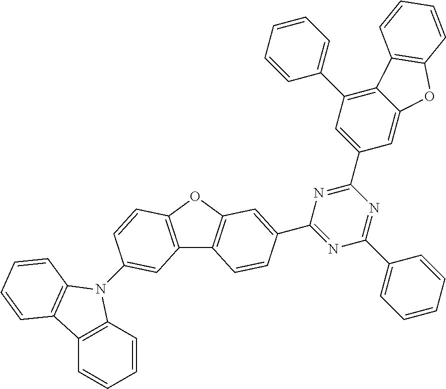 [SMILES: c1ccc(-c2nc(-c3ccc4c(c3)oc3ccc(-n5c6ccccc6c6ccccc65)cc34)nc(-c3cc(-c4ccccc4)c4c(c3)oc3ccccc34)n2)cc1] | 87% |
| 1-270 | 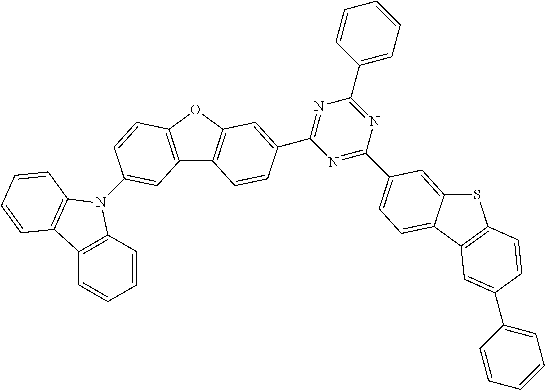 [SMILES: c1ccc(-c2ccc3sc4cc(-c5nc(-c6ccccc6)nc(-c6ccc7c(c6)oc6ccc(-n8c9ccccc9c9ccccc98)cc67)n5)ccc4c3c2)cc1] | 82% |
| 1-273 | 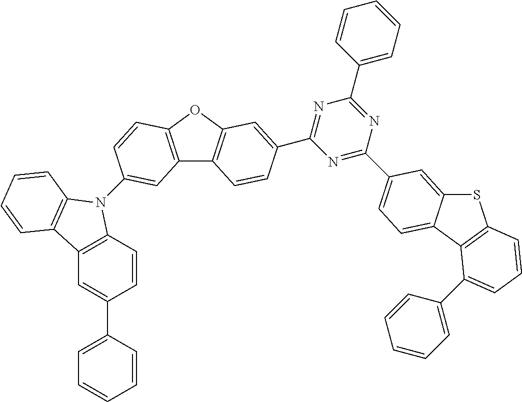 [SMILES: c1ccc(-c2ccc3c(c2)c2ccccc2n3-c2ccc3oc4cc(-c5nc(-c6ccccc6)nc(-c6ccc7c(c6)sc6cccc(-c8ccccc8)c67)n5)ccc4c3c2)cc1] | 83% |
| 1-274 | 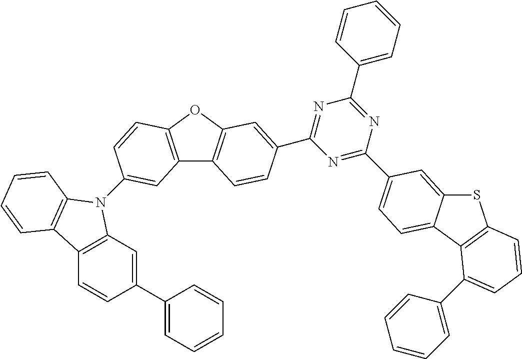 [SMILES: c1ccc(-c2ccc3c4ccccc4n(-c4ccc5oc6cc(-c7nc(-c8ccccc8)nc(-c8ccc9c(c8)sc8cccc(-c%10ccccc%10)c89)n7)ccc6c5c4)c3c2)cc1] | 85% |
| 1-276 | 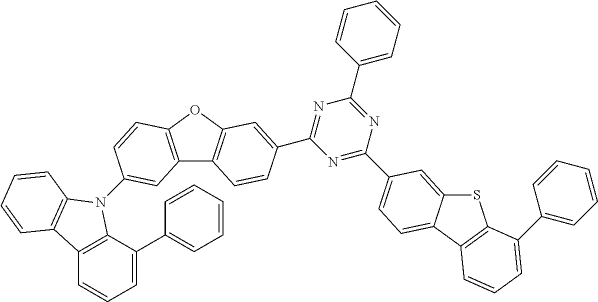 [SMILES: c1ccc(-c2nc(-c3ccc4c(c3)oc3ccc(-n5c6ccccc6c6cccc(-c7ccccc7)c65)cc34)nc(-c3ccc4c(c3)sc3c(-c5ccccc5)cccc34)n2)cc1] | 92% |
| 1-279 | 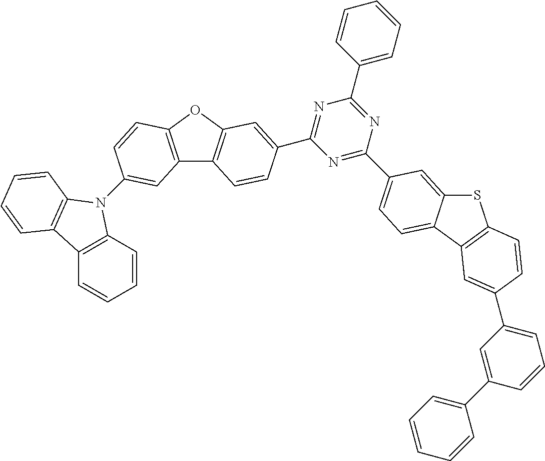 [SMILES: c1ccc(-c2cccc(-c3ccc4sc5cc(-c6nc(-c7ccccc7)nc(-c7ccc8c(c7)oc7ccc(-n9c%10ccccc%10c%10ccccc%109)cc78)n6)ccc5c4c3)c2)cc1] | 95% |
| 1-280 | 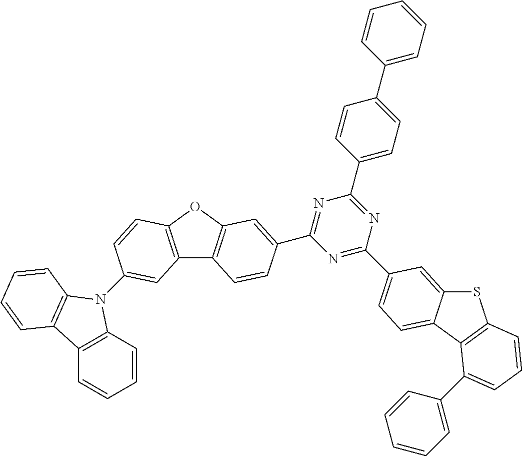 [SMILES: c1ccc(-c2ccc(-c3nc(-c4ccc5c(c4)oc4ccc(-n6c7ccccc7c7ccccc76)cc45)nc(-c4ccc5c(c4)sc4cccc(-c6ccccc6)c45)n3)cc2)cc1] | 97% |
| 1-287 | 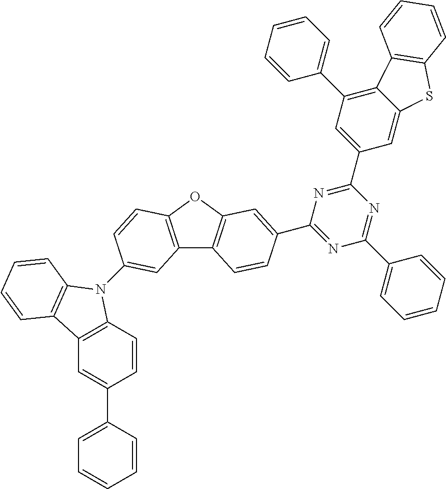 [SMILES: c1ccc(-c2ccc3c(c2)c2ccccc2n3-c2ccc3oc4cc(-c5nc(-c6ccccc6)nc(-c6cc(-c7ccccc7)c7c(c6)sc6ccccc67)n5)ccc4c3c2)cc1] | 88% |
| 1-301 | 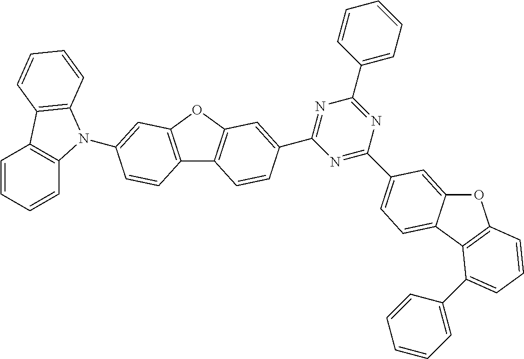 [SMILES: c1ccc(-c2nc(-c3ccc4c(c3)oc3cc(-n5c6ccccc6c6ccccc65)ccc34)nc(-c3ccc4c(c3)oc3cccc(-c5ccccc5)c34)n2)cc1] | 79% |
| 1-302 | 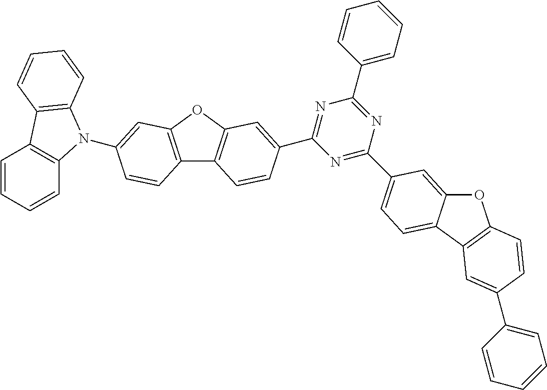 [SMILES: c1ccc(-c2ccc3oc4cc(-c5nc(-c6ccccc6)nc(-c6ccc7c(c6)oc6cc(-n8c9ccccc9c9ccccc98)ccc67)n5)ccc4c3c2)cc1] | 59% |
| 1-305 | 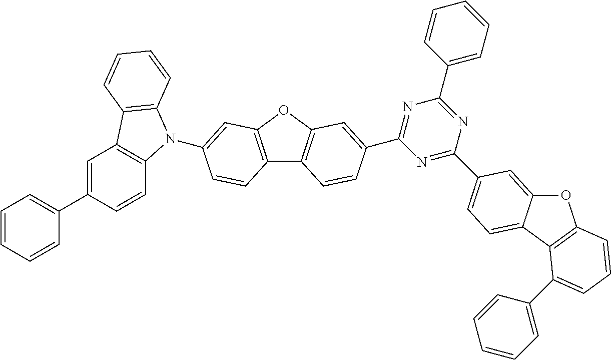 [SMILES: c1ccc(-c2ccc3c(c2)c2ccccc2n3-c2ccc3c(c2)oc2cc(-c4nc(-c5ccccc5)nc(-c5ccc6c(c5)oc5cccc(-c7ccccc7)c56)n4)ccc23)cc1] | 55% |
| 1-310 | 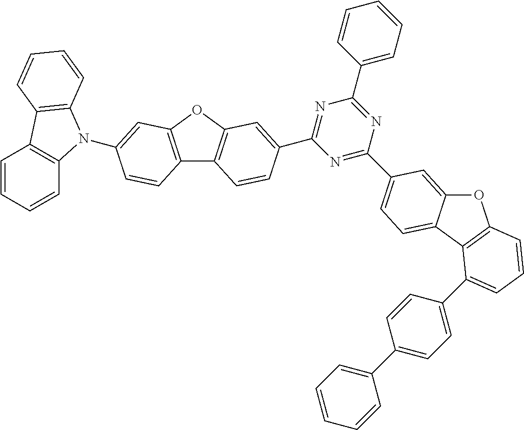 [SMILES: c1ccc(-c2ccc(-c3cccc4oc5cc(-c6nc(-c7ccccc7)nc(-c7ccc8c(c7)oc7cc(-n9c%10ccccc%10c%10ccccc%109)ccc78)n6)ccc5c34)cc2)cc1] | 89% |
| 1-312 | 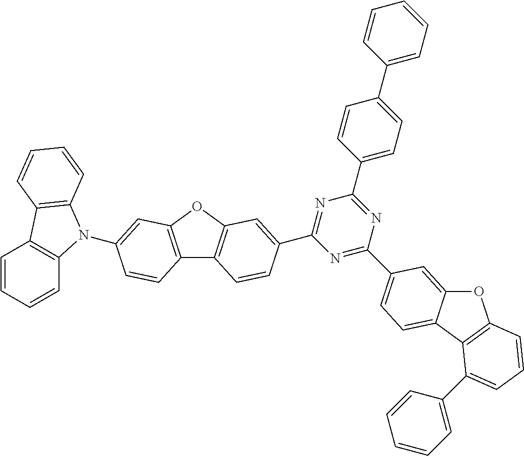 [SMILES: c1ccc(-c2ccc(-c3nc(-c4ccc5c(c4)oc4cc(-n6c7ccccc7c7ccccc76)ccc45)nc(-c4ccc5c(c4)oc4cccc(-c6ccccc6)c45)n3)cc2)cc1] | 83% |
| 1-313 | 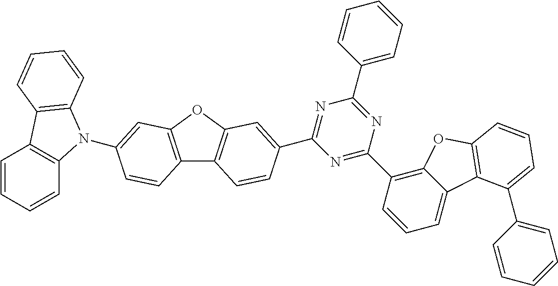 [SMILES: c1ccc(-c2nc(-c3ccc4c(c3)oc3cc(-n5c6ccccc6c6ccccc65)ccc34)nc(-c3cccc4c3oc3cccc(-c5ccccc5)c34)n2)cc1] | 84% |
| 1-316 | 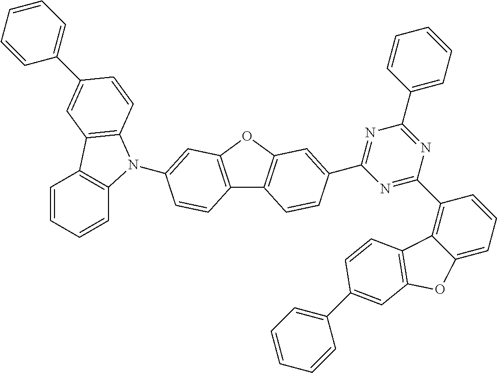 [SMILES: c1ccc(-c2ccc3c(c2)oc2cccc(-c4nc(-c5ccccc5)nc(-c5ccc6c(c5)oc5cc(-n7c8ccccc8c8cc(-c9ccccc9)ccc87)ccc56)n4)c23)cc1] | 87% |
| 1-320 | 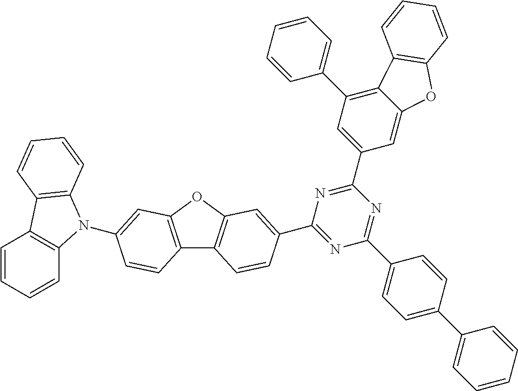 [SMILES: c1ccc(-c2ccc(-c3nc(-c4ccc5c(c4)oc4cc(-n6c7ccccc7c7ccccc76)ccc45)nc(-c4cc(-c5ccccc5)c5c(c4)oc4ccccc45)n3)cc2)cc1] | 86% |
| 1-321 | 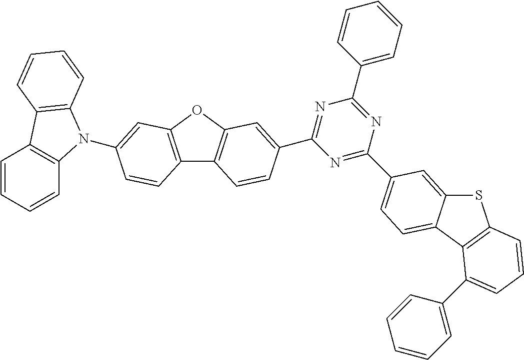 [SMILES: c1ccc(-c2nc(-c3ccc4c(c3)oc3cc(-n5c6ccccc6c6ccccc65)ccc34)nc(-c3ccc4c(c3)sc3cccc(-c5ccccc5)c34)n2)cc1] | 76% |
| 1-324 | 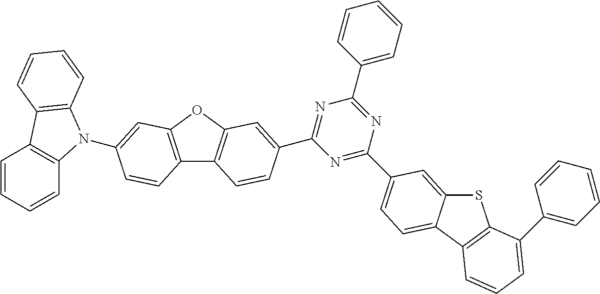 [SMILES: c1ccc(-c2nc(-c3ccc4c(c3)oc3cc(-n5c6ccccc6c6ccccc65)ccc34)nc(-c3ccc4c(c3)sc3c(-c5ccccc5)cccc34)n2)cc1] | 65% |
| 1-326 | 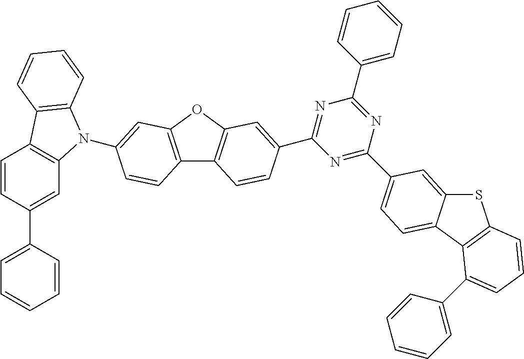 [SMILES: c1ccc(-c2ccc3c4ccccc4n(-c4ccc5c(c4)oc4cc(-c6nc(-c7ccccc7)nc(-c7ccc8c(c7)sc7cccc(-c9ccccc9)c78)n6)ccc45)c3c2)cc1] | 61% |
| 1-330 | 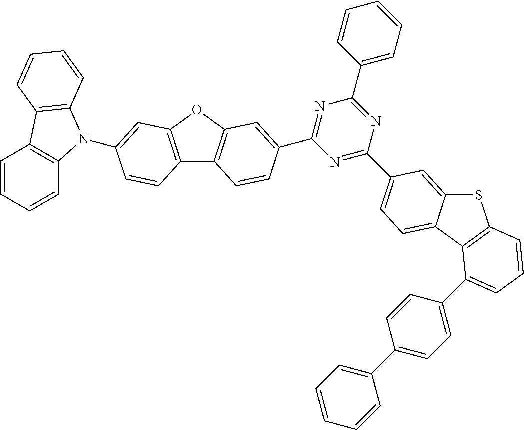 [SMILES: c1ccc(-c2ccc(-c3cccc4sc5cc(-c6nc(-c7ccccc7)nc(-c7ccc8c(c7)oc7cc(-n9c%10ccccc%10c%10ccccc%109)ccc78)n6)ccc5c34)cc2)cc1] | 69% |
| 1-333 | 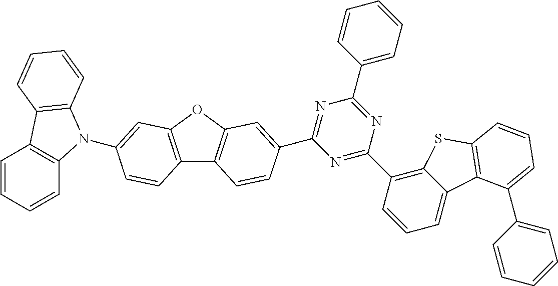 [SMILES: c1ccc(-c2nc(-c3ccc4c(c3)oc3cc(-n5c6ccccc6c6ccccc65)ccc34)nc(-c3cccc4c3sc3cccc(-c5ccccc5)c34)n2)cc1] | 87% |
| 1-337 | 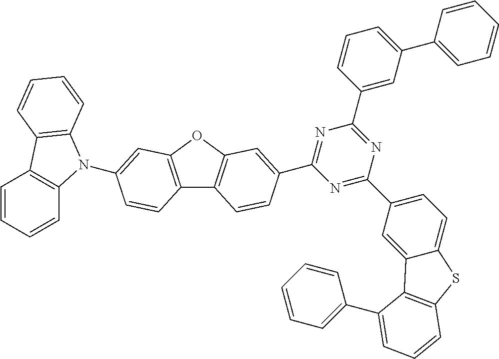 [SMILES: c1ccc(-c2cccc(-c3nc(-c4ccc5c(c4)oc4cc(-n6c7ccccc7c7ccccc76)ccc45)nc(-c4ccc5sc6cccc(-c7ccccc7)c6c5c4)n3)c2)cc1] | 65% |
| 1-355 | 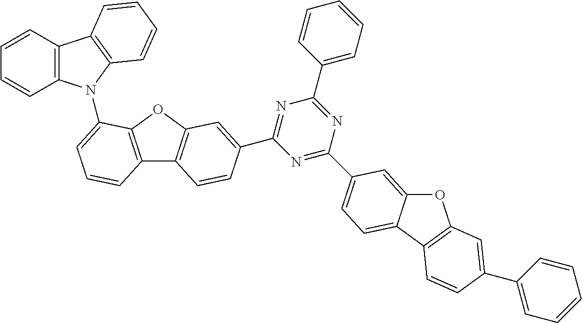 [SMILES: c1ccc(-c2ccc3c(c2)oc2cc(-c4nc(-c5ccccc5)nc(-c5ccc6c(c5)oc5c(-n7c8ccccc8c8ccccc87)cccc56)n4)ccc23)cc1] | 74% |
| 1-356 | 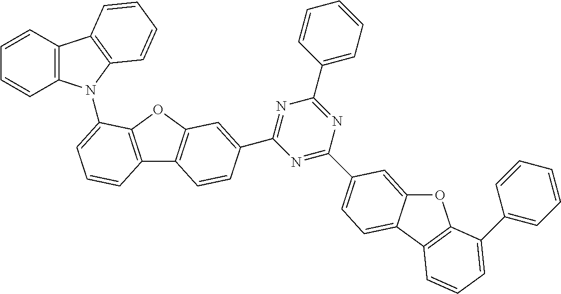 [SMILES: c1ccc(-c2nc(-c3ccc4c(c3)oc3c(-c5ccccc5)cccc34)nc(-c3ccc4c(c3)oc3c(-n5c6ccccc6c6ccccc65)cccc34)n2)cc1] | 76% |
| 1-360 | 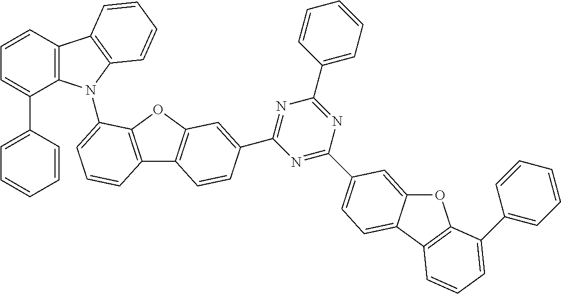 [SMILES: c1ccc(-c2nc(-c3ccc4c(c3)oc3c(-c5ccccc5)cccc34)nc(-c3ccc4c(c3)oc3c(-n5c6ccccc6c6cccc(-c7ccccc7)c65)cccc34)n2)cc1] | 72% |
| 1-364 | 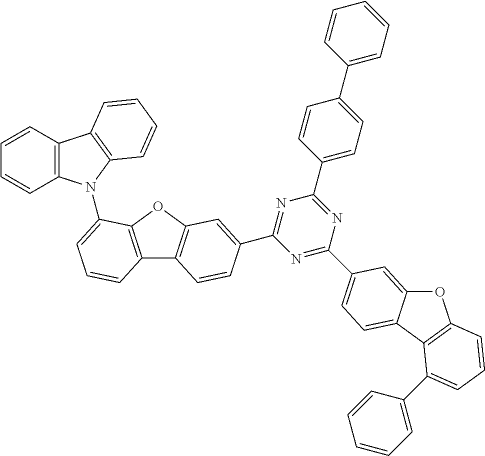 [SMILES: c1ccc(-c2ccc(-c3nc(-c4ccc5c(c4)oc4c(-n6c7ccccc7c7ccccc76)cccc45)nc(-c4ccc5c(c4)oc4cccc(-c6ccccc6)c45)n3)cc2)cc1] | 78% |
| 1-368 | 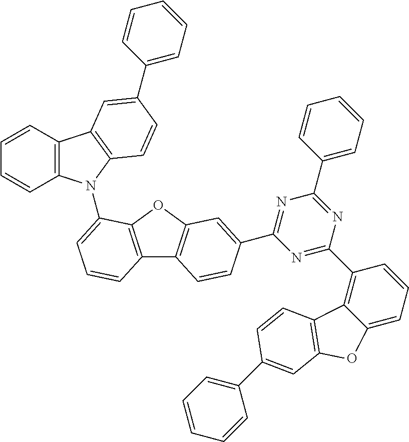 [SMILES: c1ccc(-c2ccc3c(c2)oc2cccc(-c4nc(-c5ccccc5)nc(-c5ccc6c(c5)oc5c(-n7c8ccccc8c8cc(-c9ccccc9)ccc87)cccc56)n4)c23)cc1] | 83% |
| 1-372 | 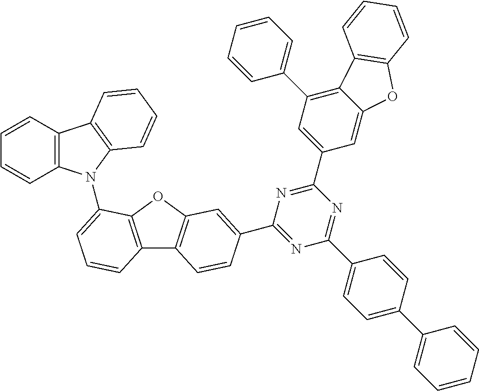 [SMILES: c1ccc(-c2ccc(-c3nc(-c4ccc5c(c4)oc4c(-n6c7ccccc7c7ccccc76)cccc45)nc(-c4cc(-c5ccccc5)c5c(c4)oc4ccccc45)n3)cc2)cc1] | 85% |
| 1-373 | 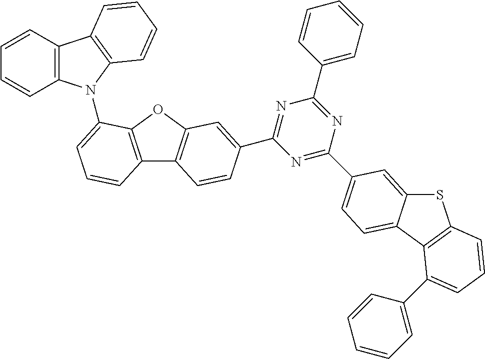 [SMILES: c1ccc(-c2nc(-c3ccc4c(c3)oc3c(-n5c6ccccc6c6ccccc65)cccc34)nc(-c3ccc4c(c3)sc3cccc(-c5ccccc5)c34)n2)cc1] | 93% |
| 1-379 | 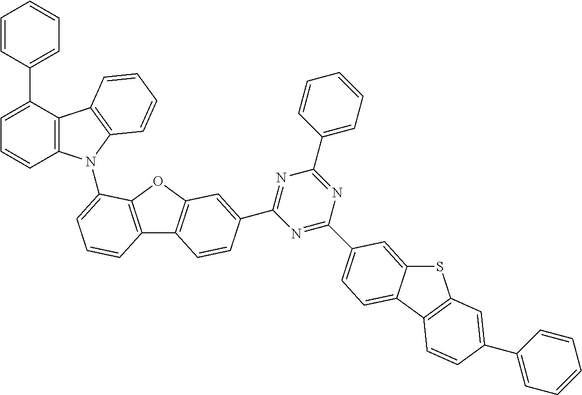 [SMILES: c1ccc(-c2ccc3c(c2)sc2cc(-c4nc(-c5ccccc5)nc(-c5ccc6c(c5)oc5c(-n7c8ccccc8c8c(-c9ccccc9)cccc87)cccc56)n4)ccc23)cc1] | 94% |
| 1-381 | 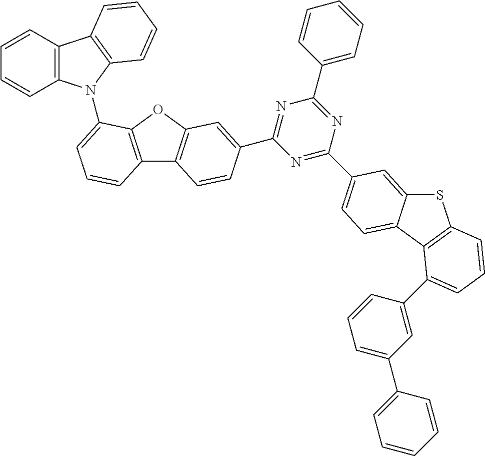 [SMILES: c1ccc(-c2cccc(-c3cccc4sc5cc(-c6nc(-c7ccccc7)nc(-c7ccc8c(c7)oc7c(-n9c%10ccccc%10c%10ccccc%109)cccc78)n6)ccc5c34)c2)cc1] | 78% |
| 1-388 | 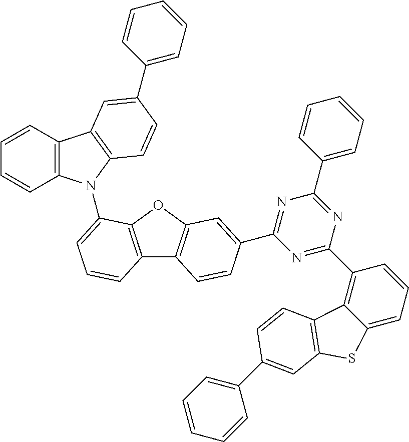 [SMILES: c1ccc(-c2ccc3c(c2)sc2cccc(-c4nc(-c5ccccc5)nc(-c5ccc6c(c5)oc5c(-n7c8ccccc8c8cc(-c9ccccc9)ccc87)cccc56)n4)c23)cc1] | 82% |

### <Preparation Example 3> Preparation of Compound 1-19

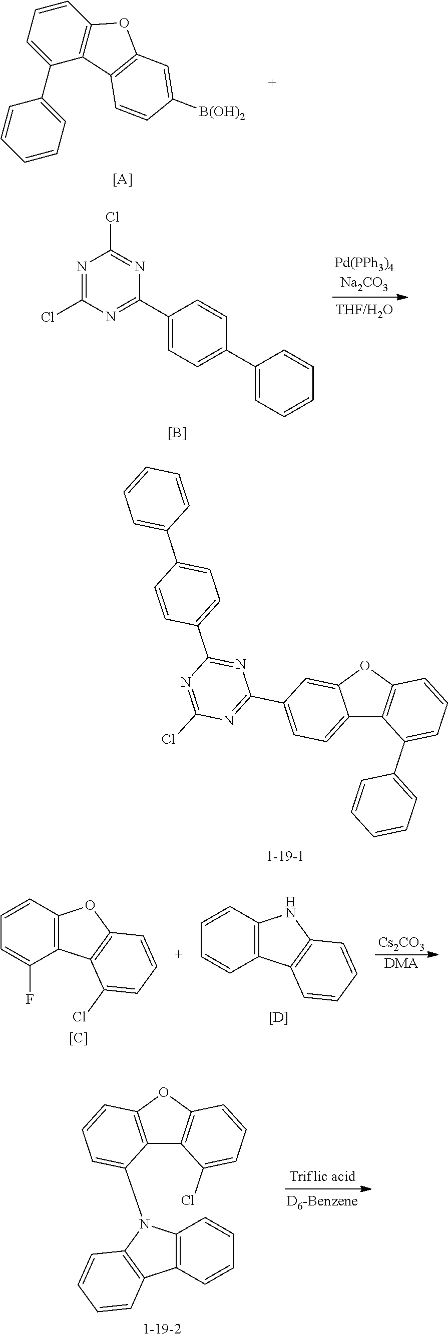

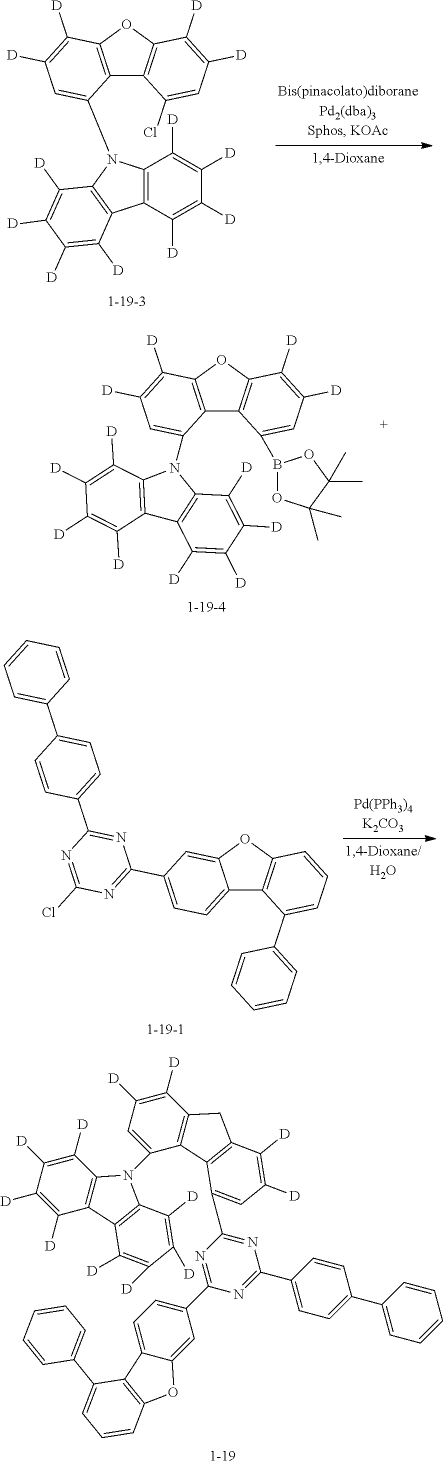

### 1) Preparation of Compound 1-19-1

In a one-neck round bottom flask, a mixture of (9-phenyldibenzo[b,d]furan-3-yl)boronic acid (8.36 g, 0.029 mol), 2-([1,1′-biphenyl]-4-yl)-4,6-dichloro-1,3,5-triazine (8.76 g, 0.029 mol), Pd(PPh₃)₄ (1.68 g, 0.0015 mol), Na₂CO₃ (6.15 g, 0.058 mol) and THE (80 mL)/H₂O (20 mL) was refluxed at 100° C. After the reaction was completed, the result was cooled and then extracted, and the organic layer was silica gel filtered to obtain Compound 1-19-1 (10.80 g, yield 73%).

### 2) Preparation of Compound 1-19-2

In a one-neck round bottom flask, a mixture of 1-chloro-9-fluorodibenzo[b,d]furan (8 g, 0.036 mol), 9H-carbazole (6.02 g, 0.036 mol), Cs₂CO₃ (23.5 g, 0.072 mol) and dimethylacetamide (80 mL) was refluxed at 185° C. After the reaction was completed, the result was cooled and then filtered to remove Cs₂CO₃, and an organic solution obtained by filtering the result was silica gel filtered to obtain Compound 1-19-2 (10.30 g, yield 79%).

### 3) Preparation of Compound 1-19-3

In a one-neck round bottom flask, a mixture of 9-(9-chlorodibenzo[b,d]furan-1-yl)-9H-carbazole (10.30 g, 0.028 mol), triflic acid (63 g, 0.42 mol) and D6-benzene (100 mL) was refluxed at room temperature (RT). After the reaction was completed, the result was quenched by slowly introducing H₂O thereto, and extracted to obtain an organic solution. The organic solution was silica gel filtered to obtain Compound 1-19-3 (10.32 g, yield 97%).

### 4) Preparation of Compound 1-19-4

In a one-neck round bottom flask, a mixture of 9-(9-chlorodibenzo[b,d]furan-1-yl-3,4,6,7-d4)-9H-carbazole-1,2,3,4,5,6,7,8-d8 (10.32 g, 0.027 mol), bis(pinacolato)diboron (13.71 g, 0.054 mol), Pd₂(dba)₃ (1.24 g, 0.0014 mol), SPhos (1.11 g, 0.0027 mol), potassium acetate (5.25 g, 0.054 mol) and 1,4-dioxane (100 mL) was refluxed at 120° C. After the reaction was completed, the result was cooled, then concentrated, and silica gel filtered to obtain Compound 1-19-4 (10.44 g, yield 82%).

### 5) Preparation of Compound 1-19

In a one-neck round bottom flask, a mixture of 9-(9-(4,4,5,5-tetramethyl-1,3,2-dioxaborolan-2-yl)dibenzo[b,d]furan-1-yl-3,4,6,7-d4)-9H-carbazole-1,2,3,4,5,6,7,8-d8 (10 g, 0.021 mol), 2-([1,1′-biphenyl]-4-yl)-4-chloro-6-(9-phenyldibenzo[b,d]furan-3-yl)-1,3,5-triazine (10.71 g, 0.021 mol), Pd(PPh₃)₄ (1.21 g, 0.0011 mol), K₂CO₃ (5.80 g, 0.042 mol) and 1,4-dioxane (100 mL)/H₂O (30 mL) was refluxed at 120° C. The result was cooled, then concentrated, and silica gel filtered to obtain Compound 1-19 (11.35 g, yield 66%).

### <Preparation Example 4> Preparation of Compounds 1-19, 1-24, 1-66, 1-67, 1-69, 1-72, 1-74, 1-76, 1-78, 1-122, 1-127, 1-131, 1-134, 1-136, 1-178, 1-180, 1-186, 1-189, 1-191, 1-233, 1-236, 1-238, 1-241, 1-245, 1-247, 1-248, 1-289, 1-296, 1-297, 1-300, 1-341, 1-347, 1-351, 1-352, 1-395, 1-400, 1-402 and 1-408

Compounds 1-19, 1-24, 1-66, 1-67, 1-69, 1-72, 1-74, 1-76, 1-78, 1-122, 1-127, 1-131, 1-134, 1-136, 1-178, 1-180, 1-186, 1-189, 1-191, 1-233, 1-236, 1-238, 1-241, 1-245, 1-247, 1-248, 1-289, 1-296, 1-297, 1-300, 1-341, 1-347, 1-351, 1-352, 1-395, 1-400, 1-402 and 1-408 were synthesized in the same manner as in Preparation Example 3 except that Intermediates A′ to D′ of the following Table 2 were used instead of A to D.

TABLE 2

| Compound | A′ | B′ | C′ | D′ | Target Compound | Yield |
|---|---|---|---|---|---|---|
| 1-19 | 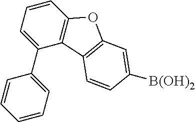 | 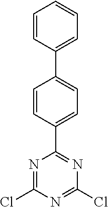 | 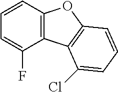 | 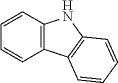 | 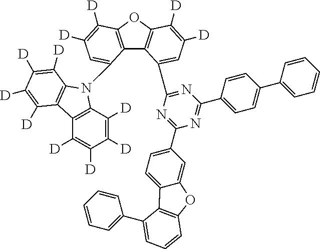 [SMILES: [2H]c1cc(-c2nc(-c3ccc(-c4ccccc4)cc3)nc(-c3ccc4c(c3)oc3cccc(-c5ccccc5)c34)n2)c2c(oc3c([2H])c([2H])cc(-n4c5c([2H])c([2H])c([2H])c([2H])c5c5c([2H])c([2H])c([2H])c([2H])c54)c32)c1[2H]] | 66% |
| 1-24 | 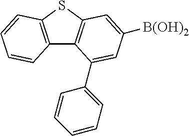 | 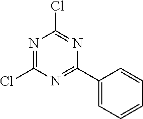 | 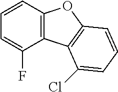 | 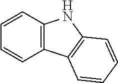 | 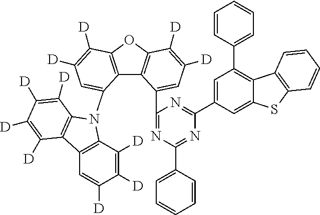 [SMILES: [2H]c1cc2c3c([2H])c([2H])c([2H])c([2H])c3n(-c3cc([2H])c([2H])c4oc5c([2H])c([2H])cc(-c6nc(-c7ccccc7)nc(-c7cc(-c8ccccc8)c8c(c7)sc7ccccc78)n6)c5c34)c2c([2H])c1[2H]] | 73% |
| 1-66 | 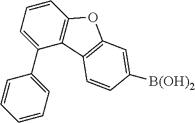 | 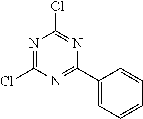 | 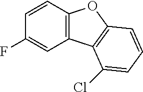 | 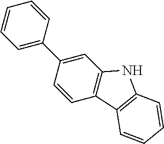 | 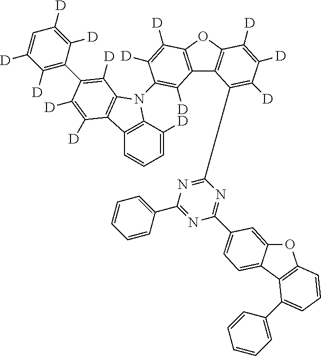 [SMILES: [2H]c1cc([2H])c([2H])c(-c2c([2H])c([2H])c3c4cccc([2H])c4n(-c4c([2H])c([2H])c5oc6c([2H])c([2H])c([2H])c(-c7nc(-c8ccccc8)nc(-c8ccc9c(c8)oc8cccc(-c%10ccccc%10)c89)n7)c6c5c4[2H])c3c2[2H])c1[2H]] | 74% |
| 1-67 | 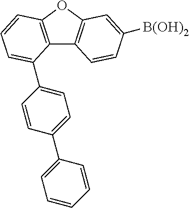 | 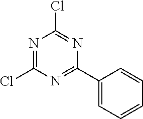 | 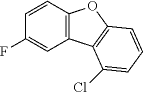 | 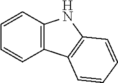 | 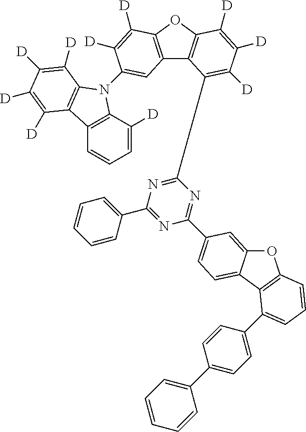 [SMILES: [2H]c1c(-n2c3c([2H])cccc3c3c([2H])c([2H])c([2H])c([2H])c32)cc2c(oc3c([2H])c([2H])c([2H])c(-c4nc(-c5ccccc5)nc(-c5ccc6c(c5)oc5cccc(-c7ccc(-c8ccccc8)cc7)c56)n4)c32)c1[2H]] | 71% |
| 1-69 | 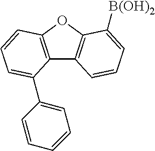 | 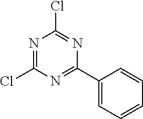 | 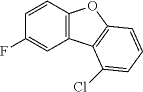 | 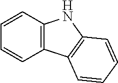 | 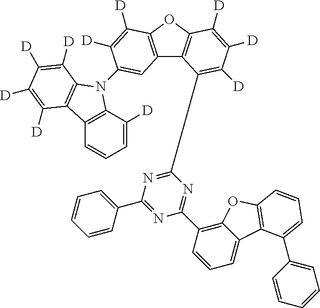 [SMILES: [2H]c1c(-n2c3c([2H])cccc3c3c([2H])c([2H])c([2H])c([2H])c32)cc2c(oc3c([2H])c([2H])c([2H])c(-c4nc(-c5ccccc5)nc(-c5cccc6c5oc5cccc(-c7ccccc7)c56)n4)c32)c1[2H]] | 69% |
| 1-72 | 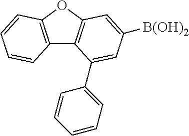 | 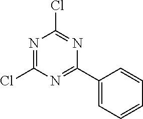 | 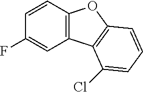 | 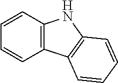 | 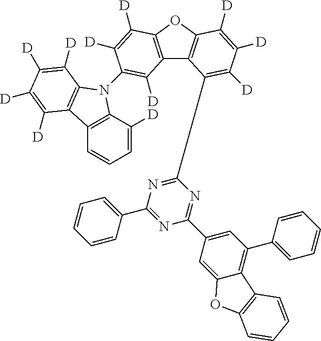 [SMILES: [2H]c1c(-n2c3c([2H])cccc3c3c([2H])c([2H])c([2H])c([2H])c32)c([2H])c2c(oc3c([2H])c([2H])c([2H])c(-c4nc(-c5ccccc5)nc(-c5cc(-c6ccccc6)c6c(c5)oc5ccccc56)n4)c32)c1[2H]] | 83% |
| 1-74 | 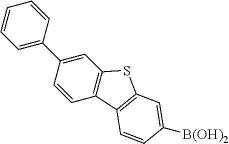 | 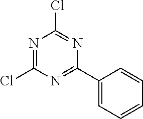 | 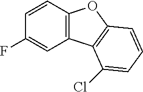 | 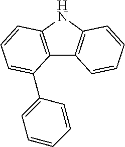 | 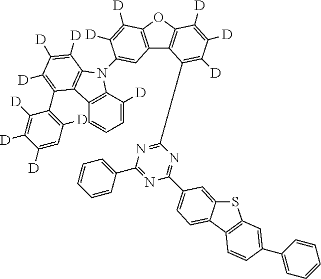 [SMILES: [2H]c1cc([2H])c(-c2c([2H])c([2H])c([2H])c3c2c2cccc([2H])c2n3-c2cc3c(oc4c([2H])c([2H])c([2H])c(-c5nc(-c6ccccc6)nc(-c6ccc7c(c6)sc6cc(-c8ccccc8)ccc67)n5)c43)c([2H])c2[2H])c([2H])c1[2H]] | 77% |
| 1-76 | 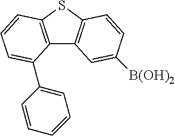 | 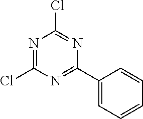 | 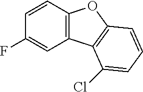 | 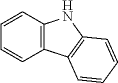 | 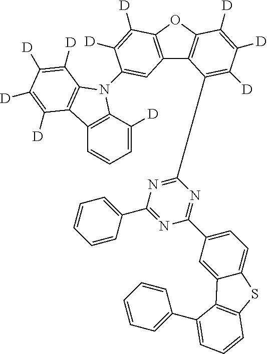 [SMILES: [2H]c1c(-n2c3c([2H])cccc3c3c([2H])c([2H])c([2H])c([2H])c32)cc2c(oc3c([2H])c([2H])c([2H])c(-c4nc(-c5ccccc5)nc(-c5ccc6sc7cccc(-c8ccccc8)c7c6c5)n4)c32)c1[2H]] | 54% |
| 1-78 | 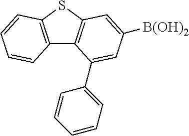 | 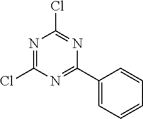 | 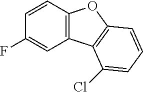 | 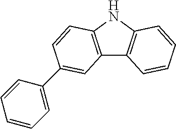 | 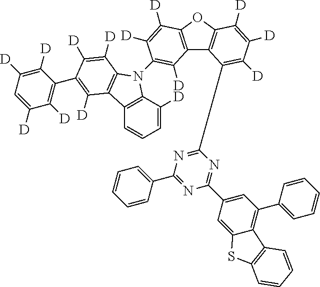 [SMILES: [2H]c1cc([2H])c([2H])c(-c2c([2H])c([2H])c3c(c2[2H])c2cccc([2H])c2n3-c2c([2H])c([2H])c3oc4c([2H])c([2H])c([2H])c(-c5nc(-c6ccccc6)nc(-c6cc(-c7ccccc7)c7c(c6)sc6ccccc67)n5)c4c3c2[2H])c1[2H]] | 98% |
| 1-122 | 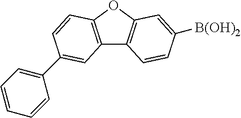 | 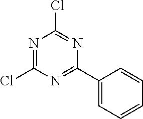 | 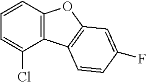 | 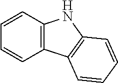 | 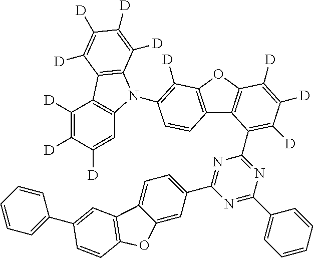 [SMILES: [2H]c1cc2c(c([2H])c1[2H])c1c([2H])c([2H])c([2H])c([2H])c1n2-c1ccc2c(oc3c([2H])c([2H])c([2H])c(-c4nc(-c5ccccc5)nc(-c5ccc6c(c5)oc5ccc(-c7ccccc7)cc56)n4)c32)c1[2H]] | 83% |
| 1-127 | 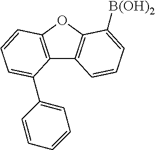 | 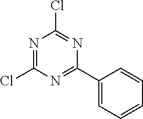 | 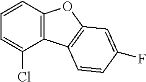 | 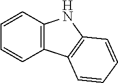 | 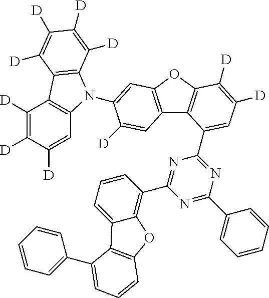 [SMILES: [2H]c1cc2c(cc1-n1c3cc([2H])c([2H])c([2H])c3c3c([2H])c([2H])c([2H])c([2H])c31)oc1c([2H])c([2H])cc(-c3nc(-c4ccccc4)nc(-c4cccc5c4oc4cccc(-c6ccccc6)c45)n3)c12] | 88% |
| 1-131 | 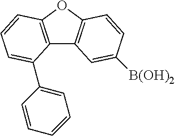 | 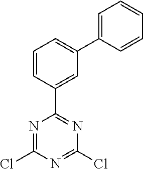 | 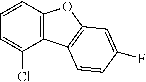 | 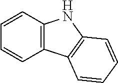 | 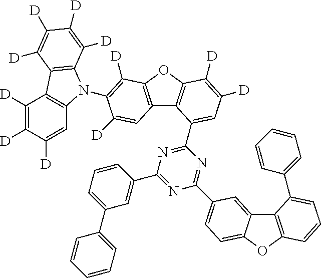 [SMILES: [2H]c1cc2c(c([2H])c1[2H])c1c([2H])c([2H])c([2H])c([2H])c1n2-c1c([2H])cc2c(oc3c([2H])c([2H])cc(-c4nc(-c5cccc(-c6ccccc6)c5)nc(-c5ccc6oc7cccc(-c8ccccc8)c7c6c5)n4)c32)c1[2H]] | 76% |
| 1-134 | 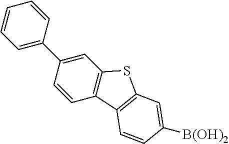 | 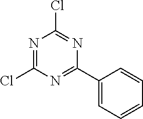 | 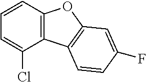 | 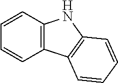 | 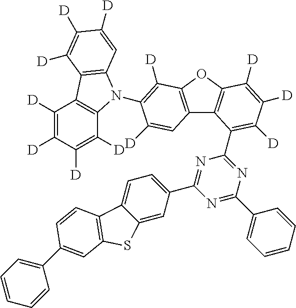 [SMILES: [2H]c1cc2c(c([2H])c1[2H])c1c([2H])c([2H])c([2H])c([2H])c1n2-c1c([2H])cc2c(oc3c([2H])c([2H])c([2H])c(-c4nc(-c5ccccc5)nc(-c5ccc6c(c5)sc5cc(-c7ccccc7)ccc56)n4)c32)c1[2H]] | 77% |
| 1-136 | 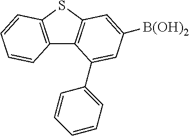 | 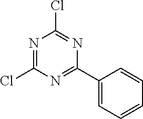 | 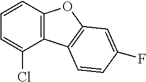 | 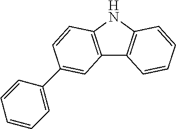 | 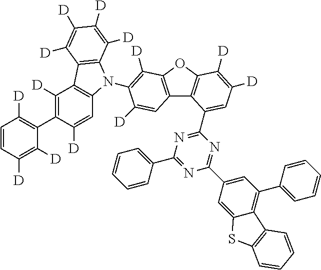 [SMILES: [2H]c1ccc([2H])c(-c2c([2H])cc3c(c2[2H])c2c([2H])c([2H])c([2H])c([2H])c2n3-c2c([2H])cc3c(oc4c([2H])c([2H])cc(-c5nc(-c6ccccc6)nc(-c6cc(-c7ccccc7)c7c(c6)sc6ccccc67)n5)c43)c2[2H])c1[2H]] | 65% |
| 1-178 | 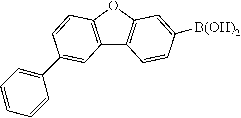 | 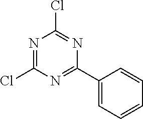 | 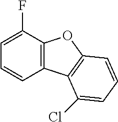 | 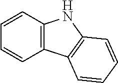 | 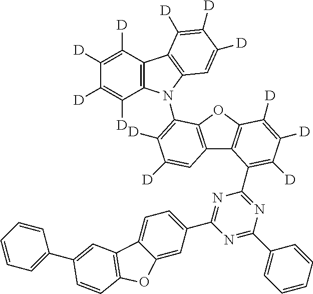 [SMILES: [2H]c1cc2c(oc3c([2H])c([2H])c([2H])c(-c4nc(-c5ccccc5)nc(-c5ccc6c(c5)oc5ccc(-c7ccccc7)cc56)n4)c32)c(-n2c3cc([2H])c([2H])c([2H])c3c3c([2H])c([2H])c([2H])c([2H])c32)c1[2H]] | 63% |
| 1-180 | 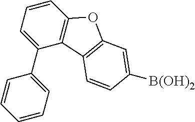 | 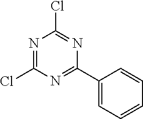 | 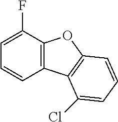 | 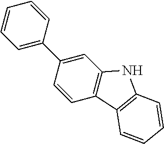 | 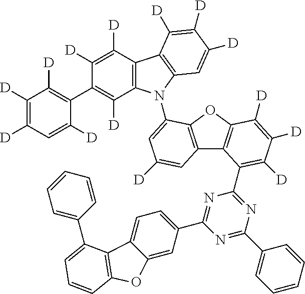 [SMILES: [2H]c1cc(-n2c3cc([2H])c([2H])c([2H])c3c3c([2H])c([2H])c(-c4c([2H])cc([2H])c([2H])c4[2H])c([2H])c32)c2oc3c([2H])c([2H])c([2H])c(-c4nc(-c5ccccc5)nc(-c5ccc6c(c5)oc5cccc(-c7ccccc7)c56)n4)c3c2c1] | 74% |
| 1-186 | 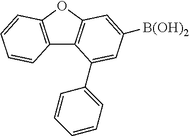 | 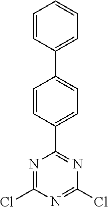 | 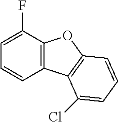 | 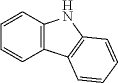 | 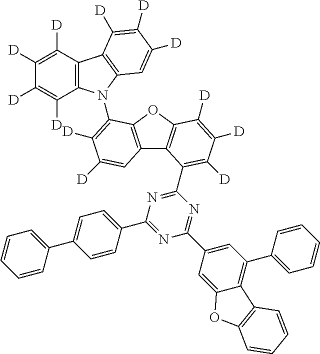 [SMILES: [2H]c1cc2c(oc3c([2H])c([2H])c([2H])c(-c4nc(-c5ccc(-c6ccccc6)cc5)nc(-c5cc(-c6ccccc6)c6c(c5)oc5ccccc56)n4)c32)c(-n2c3cc([2H])c([2H])c([2H])c3c3c([2H])c([2H])c([2H])c([2H])c32)c1[2H]] | 78% |
| 1-189 | 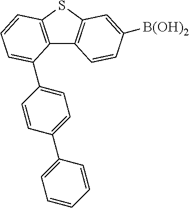 | 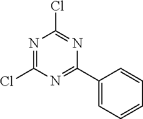 | 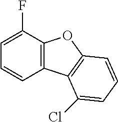 | 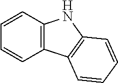 | 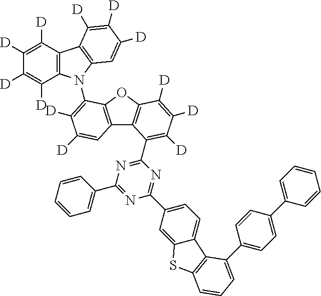 [SMILES: [2H]c1cc2c(oc3c([2H])c([2H])c([2H])c(-c4nc(-c5ccccc5)nc(-c5ccc6c(c5)sc5cccc(-c7ccc(-c8ccccc8)cc7)c56)n4)c32)c(-n2c3cc([2H])c([2H])c([2H])c3c3c([2H])c([2H])c([2H])c([2H])c32)c1[2H]] | 88% |
| 1-191 | 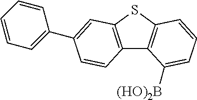 | 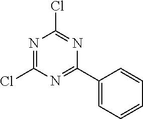 | 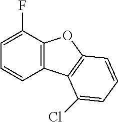 | 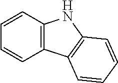 | 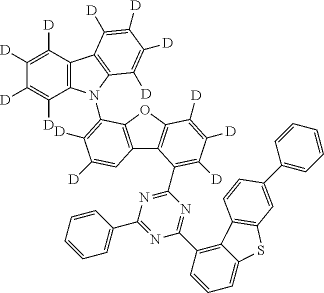 [SMILES: [2H]c1cc2c(oc3c([2H])c([2H])c([2H])c(-c4nc(-c5ccccc5)nc(-c5cccc6sc7cc(-c8ccccc8)ccc7c56)n4)c32)c(-n2c3c([2H])c([2H])c([2H])c([2H])c3c3c([2H])c([2H])c([2H])c([2H])c32)c1[2H]] | 77% |
| 1-233 | 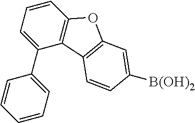 | 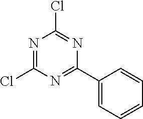 | 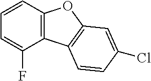 | 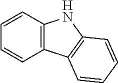 | 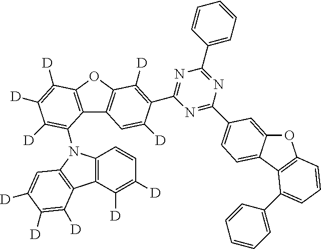 [SMILES: [2H]c1cc2c(c([2H])c1[2H])c1c([2H])c([2H])ccc1n2-c1c([2H])c([2H])c([2H])c2oc3c([2H])c(-c4nc(-c5ccccc5)nc(-c5ccc6c(c5)oc5cccc(-c7ccccc7)c56)n4)c([2H])cc3c12] | 87% |
| 1-236 | 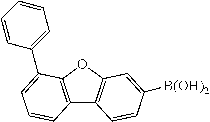 | 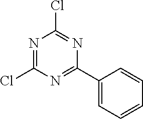 | 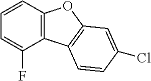 | 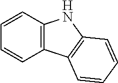 | 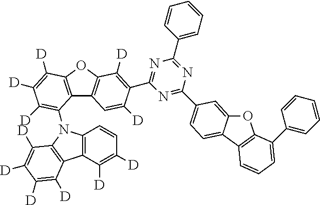 [SMILES: [2H]c1cc2c(oc3c([2H])c([2H])c([2H])c(-n4c5ccc([2H])c([2H])c5c5c([2H])c([2H])c([2H])c([2H])c54)c32)c([2H])c1-c1nc(-c2ccccc2)nc(-c2ccc3c(c2)oc2c(-c4ccccc4)cccc23)n1] | 80% |
| 1-238 | 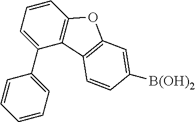 | 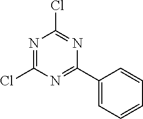 | 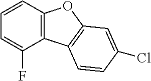 | 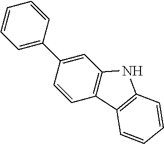 | 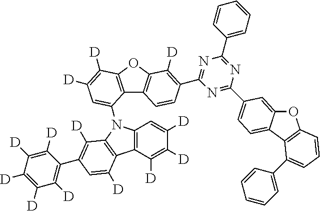 [SMILES: [2H]c1cc2c(c([2H])c1[2H])c1c([2H])cc(-c3c([2H])c([2H])c([2H])c([2H])c3[2H])c([2H])c1n2-c1cc([2H])c([2H])c2oc3c([2H])c(-c4nc(-c5ccccc5)nc(-c5ccc6c(c5)oc5cccc(-c7ccccc7)c56)n4)ccc3c12] | 69% |
| 1-241 | 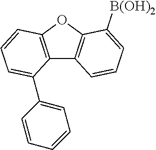 | 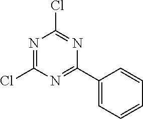 | 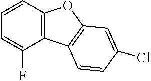 | 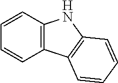 | 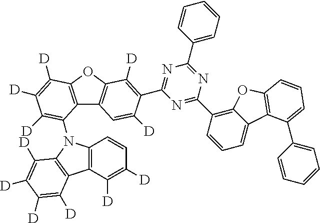 [SMILES: [2H]c1cc2c(oc3c([2H])c([2H])c([2H])c(-n4c5ccc([2H])c([2H])c5c5c([2H])c([2H])c([2H])c([2H])c54)c32)c([2H])c1-c1nc(-c2ccccc2)nc(-c2cccc3c2oc2cccc(-c4ccccc4)c23)n1] | 65% |
| 1-245 | 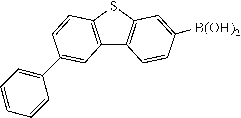 | 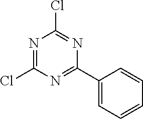 | 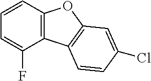 | 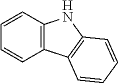 | 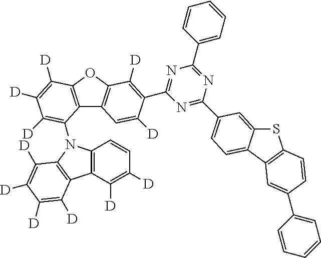 [SMILES: [2H]c1cc2c(oc3c([2H])c([2H])c([2H])c(-n4c5ccc([2H])c([2H])c5c5c([2H])c([2H])c([2H])c([2H])c54)c32)c([2H])c1-c1nc(-c2ccccc2)nc(-c2ccc3c(c2)sc2ccc(-c4ccccc4)cc23)n1] | 69% |
| 1-247 | 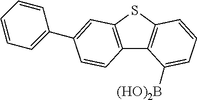 | 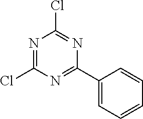 | 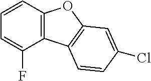 | 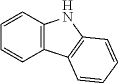 | 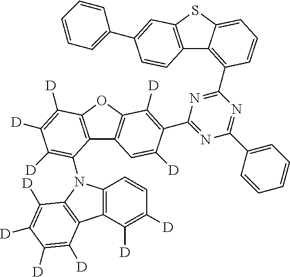 [SMILES: [2H]c1cc2c(oc3c([2H])c([2H])c([2H])c(-n4c5ccc([2H])c([2H])c5c5c([2H])c([2H])c([2H])c([2H])c54)c32)c([2H])c1-c1nc(-c2ccccc2)nc(-c2cccc3sc4cc(-c5ccccc5)ccc4c23)n1] | 73% |
| 1-248 | 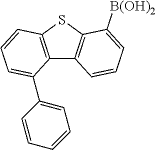 | 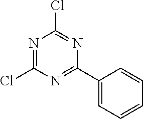 | 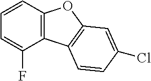 | 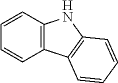 | 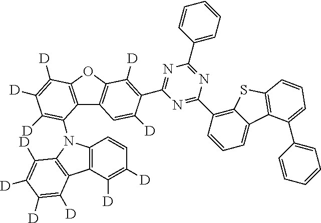 [SMILES: [2H]c1cc2c(oc3c([2H])c([2H])c([2H])c(-n4c5ccc([2H])c([2H])c5c5c([2H])c([2H])c([2H])c([2H])c54)c32)c([2H])c1-c1nc(-c2ccccc2)nc(-c2cccc3c2sc2cccc(-c4ccccc4)c23)n1] | 70% |
| 1-289 | 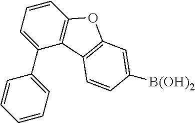 | 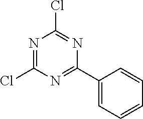 | 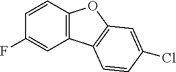 | 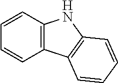 | 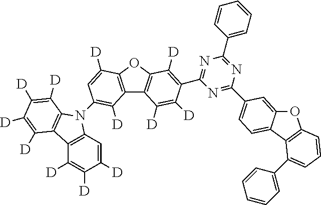 [SMILES: [2H]c1cc2c(c([2H])c1[2H])c1c([2H])c([2H])c([2H])c([2H])c1n2-c1cc([2H])c2oc3c([2H])c(-c4nc(-c5ccccc5)nc(-c5ccc6c(c5)oc5cccc(-c7ccccc7)c56)n4)c([2H])c([2H])c3c2c1[2H]] | 74% |
| 1-296 | 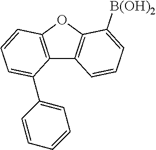 | 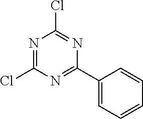 | 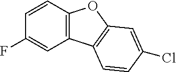 | 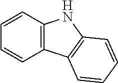 | 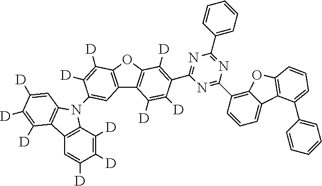 [SMILES: [2H]c1cc2c(c([2H])c1[2H])c1cc([2H])c([2H])c([2H])c1n2-c1cc2c(oc3c([2H])c(-c4nc(-c5ccccc5)nc(-c5cccc6c5oc5cccc(-c7ccccc7)c56)n4)c([2H])c([2H])c32)c([2H])c1[2H]] | 73% |
| 1-297 | 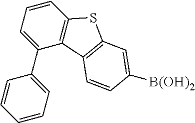 | 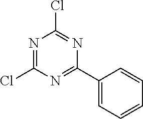 | 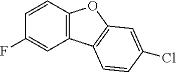 | 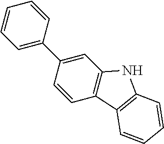 | 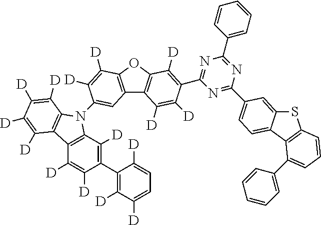 [SMILES: [2H]c1ccc([2H])c(-c2c([2H])c([2H])c3c4c([2H])c([2H])c([2H])c([2H])c4n(-c4cc5c(oc6c([2H])c(-c7nc(-c8ccccc8)nc(-c8ccc9c(c8)sc8cccc(-c%10ccccc%10)c89)n7)c([2H])c([2H])c65)c([2H])c4[2H])c3c2[2H])c1[2H]] | 65% |
| 1-300 | 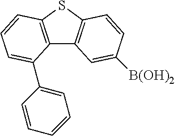 | 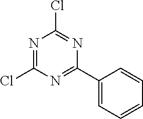 | 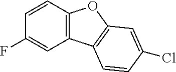 | 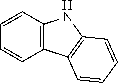 | 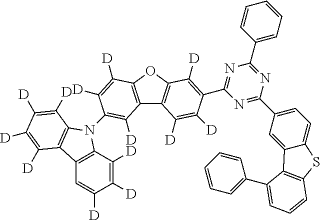 [SMILES: [2H]c1cc2c3c([2H])c([2H])c([2H])c([2H])c3n(-c3c([2H])c([2H])c4oc5c([2H])c(-c6nc(-c7ccccc7)nc(-c7ccc8sc9cccc(-c%10ccccc%10)c9c8c7)n6)c([2H])c([2H])c5c4c3[2H])c2c([2H])c1[2H]] | 67% |
| 1-341 | 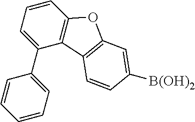 | 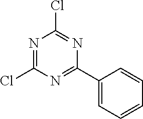 | 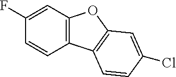 | 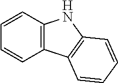 | 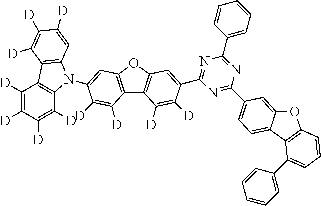 [SMILES: [2H]c1cc2c(c([2H])c1[2H])c1c([2H])c([2H])c([2H])c([2H])c1n2-c1cc2oc3cc(-c4nc(-c5ccccc5)nc(-c5ccc6c(c5)oc5cccc(-c7ccccc7)c56)n4)c([2H])c([2H])c3c2c([2H])c1[2H]] | 59% |
| 1-347 | 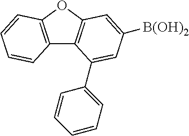 | 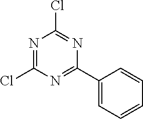 | 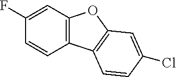 | 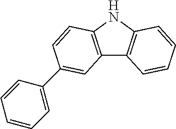 | 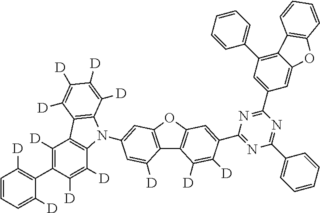 [SMILES: [2H]c1cccc([2H])c1-c1c([2H])c([2H])c2c(c1[2H])c1c([2H])c([2H])c([2H])c([2H])c1n2-c1cc([2H])c2c(c1)oc1cc(-c3nc(-c4ccccc4)nc(-c4cc(-c5ccccc5)c5c(c4)oc4ccccc45)n3)c([2H])c([2H])c12] | 92% |
| 1-351 | 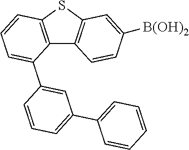 | 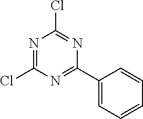 | 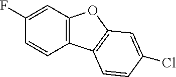 | 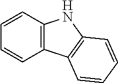 | 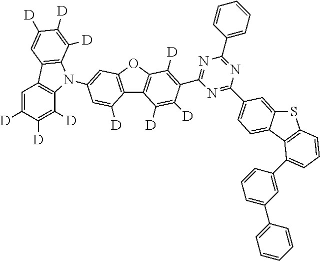 [SMILES: [2H]c1cc2c3cc([2H])c([2H])c([2H])c3n(-c3cc([2H])c4c(c3)oc3c([2H])c(-c5nc(-c6ccccc6)nc(-c6ccc7c(c6)sc6cccc(-c8cccc(-c9ccccc9)c8)c67)n5)c([2H])c([2H])c34)c2c([2H])c1[2H]] | 91% |
| 1-352 | 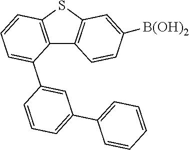 | 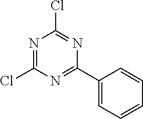 | 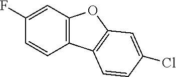 | 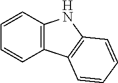 | 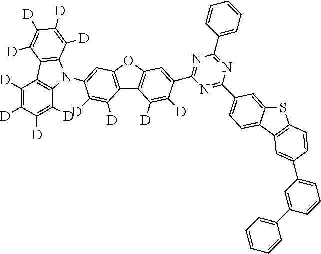 [SMILES: [2H]c1c(-c2nc(-c3ccccc3)nc(-c3ccc4c(c3)sc3ccc(-c5cccc(-c6ccccc6)c5)cc34)n2)cc2oc3cc(-n4c5c([2H])c([2H])c([2H])c([2H])c5c5c([2H])c([2H])c([2H])c([2H])c54)c([2H])c([2H])c3c2c1[2H]] | 88% |
| 1-395 | 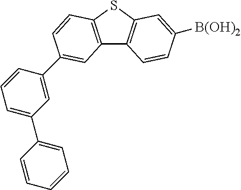 | 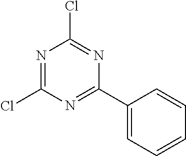 | 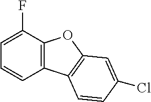 | 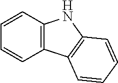 | 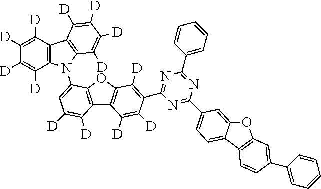 [SMILES: [2H]c1cc(-n2c3c([2H])c([2H])c([2H])c([2H])c3c3c([2H])c([2H])c([2H])c([2H])c32)c2oc3c([2H])c(-c4nc(-c5ccccc5)nc(-c5ccc6c(c5)oc5cc(-c7ccccc7)ccc56)n4)c([2H])c([2H])c3c2c1[2H]] | 88% |
| 1-400 | 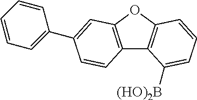 | 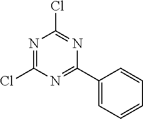 | 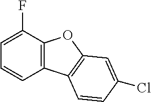 | 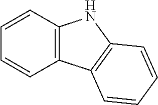 | 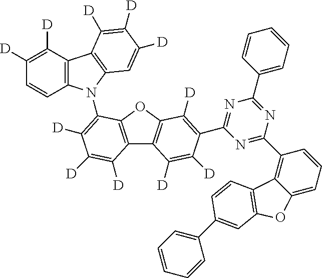 [SMILES: [2H]c1cc2c(c([2H])c1[2H])c1c([2H])c([2H])ccc1n2-c1c([2H])c([2H])c([2H])c2c1oc1c([2H])c(-c3nc(-c4ccccc4)nc(-c4cccc5oc6cc(-c7ccccc7)ccc6c45)n3)c([2H])c([2H])c12] | 85% |
| 1-402 | 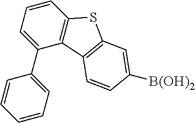 | 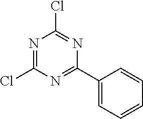 | 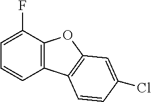 | 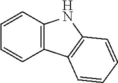 | 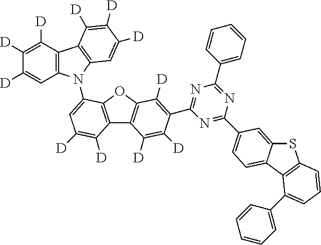 [SMILES: [2H]c1cc2c(c([2H])c1[2H])c1c([2H])c([2H])c([2H])cc1n2-c1cc([2H])c([2H])c2c1oc1c([2H])c(-c3nc(-c4ccccc4)nc(-c4ccc5c(c4)sc4cccc(-c6ccccc6)c45)n3)c([2H])c([2H])c12] | 84% |
| 1-408 | 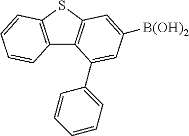 | 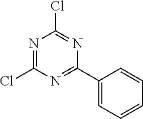 | 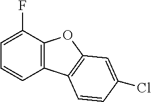 | 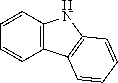 | 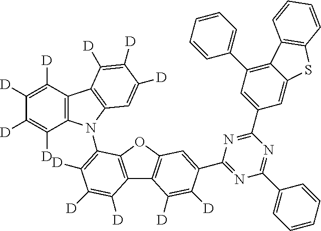 [SMILES: [2H]c1cc2c(c([2H])c1[2H])c1c([2H])c([2H])c([2H])c([2H])c1n2-c1c([2H])c([2H])c([2H])c2c1oc1cc(-c3nc(-c4ccccc4)nc(-c4cc(-c5ccccc5)c5c(c4)sc4ccccc45)n3)c([2H])c([2H])c12] | 92% |

### <Preparation Example 5> Preparation of Compound 2-1

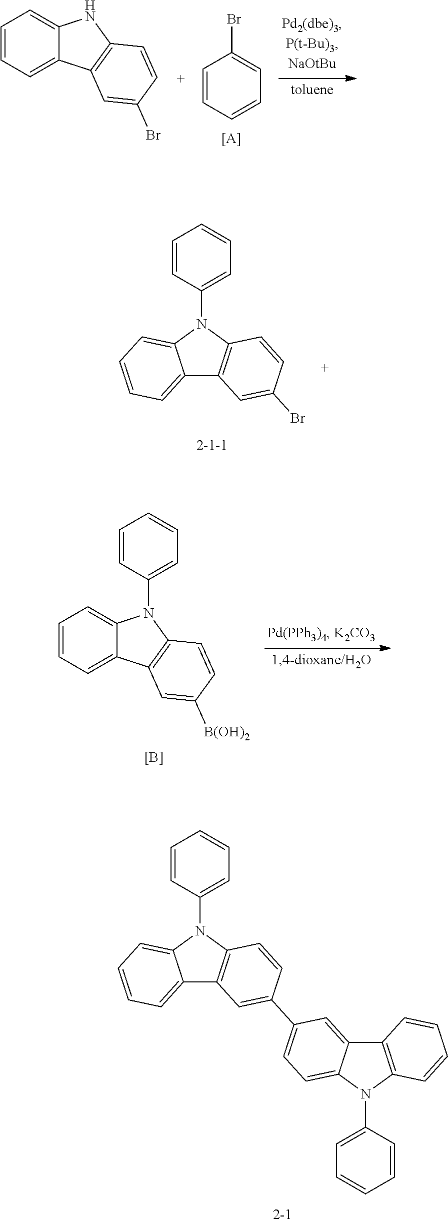

### 1) Preparation of Compound 2-1-1

In a one-neck round bottom flask, a mixture of 3-bromo-9H-carbazole (10 g, 0.041 mol), bromobenzene (6.41 g, 0.041 mol), Pd₂(dba)₃ (3.75 g, 0.0041 mol), P(t-Bu)₃ (1.66 g, 0.0082 mmol), NaOtBu (7.88 g, 0.082 mol) and toluene (100 mL) was refluxed at 100° C. After the reaction was completed, the result was cooled and then extracted, and the organic layer was silica gel filtered to obtain Compound 2-1-1 (9.51 g, yield 72%).

### 2) Preparation of Compound 2-1

In a one-neck round bottom flask, a mixture of 3-bromo-9-phenyl-9H-carbazole (9.51 g, 0.030 mol), (9-phenyl-9H-carbazol-3-yl)boronic acid (8.61 g, 0.030 mol), Pd(PPh₃)₄ (1.73 g, 0.0015 mol), K₂CO₃ (8.29 g, 0.06 mol) and 1,4-dioxane (90 mL)/H₂O (30 mL) was refluxed at 125° C. The result was cooled and then extracted, and the organic layer was column purified to obtain Compound 2-1 (9.89 g, yield 68%).

### <Preparation Example 6> Preparation of Compounds 2-2, 2-16, 2-27, 2-28 and 2-32

Compounds 2-2, 2-16, 2-27, 2-28 and 2-32 were synthesized in the same manner as in Preparation Example 5 except that Intermediates A′ and B′ of the following Table 3 were used instead of A and B.

TABLE 3

| Compound | A′ | B′ | Target Compound | Yield |
|---|---|---|---|---|
| 2-2 | 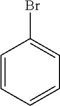 | 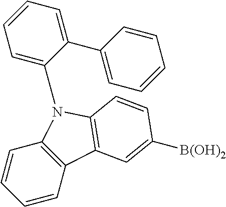 | 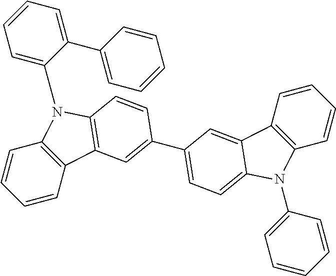 [SMILES: c1ccc(-c2ccccc2-n2c3ccccc3c3cc(-c4ccc5c(c4)c4ccccc4n5-c4ccccc4)ccc32)cc1] | 68% |
| 2-16 | 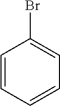 | 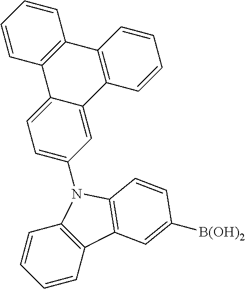 | 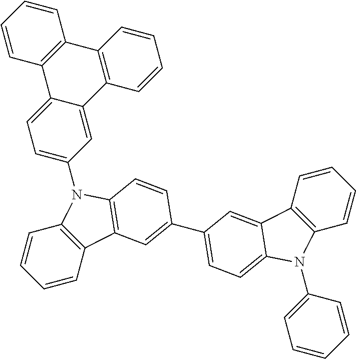 [SMILES: c1ccc(-n2c3ccccc3c3cc(-c4ccc5c(c4)c4ccccc4n5-c4ccc5c6ccccc6c6ccccc6c5c4)ccc32)cc1] | 87% |
| 2-27 | 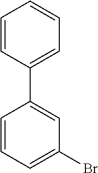 | 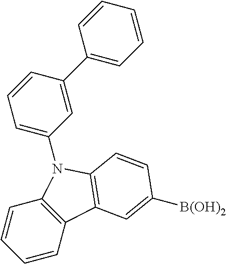 | 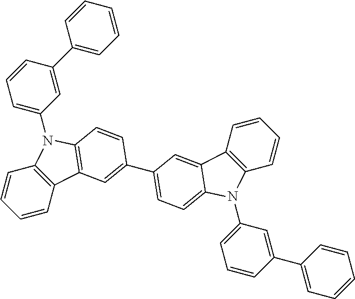 [SMILES: c1ccc(-c2cccc(-n3c4ccccc4c4cc(-c5ccc6c(c5)c5ccccc5n6-c5cccc(-c6ccccc6)c5)ccc43)c2)cc1] | 66% |
| 2-28 | 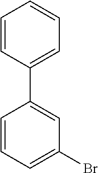 | 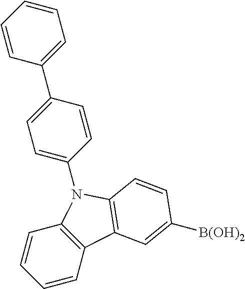 | 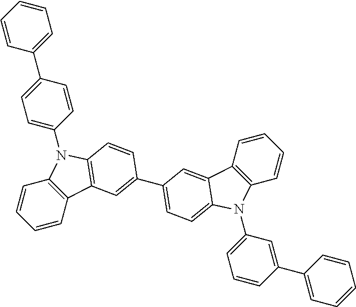 [SMILES: c1ccc(-c2ccc(-n3c4ccccc4c4cc(-c5ccc6c(c5)c5ccccc5n6-c5cccc(-c6ccccc6)c5)ccc43)cc2)cc1] | 54% |
| 2-32 | 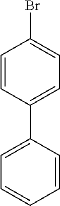 | 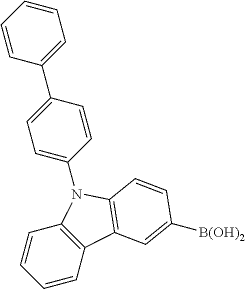 | 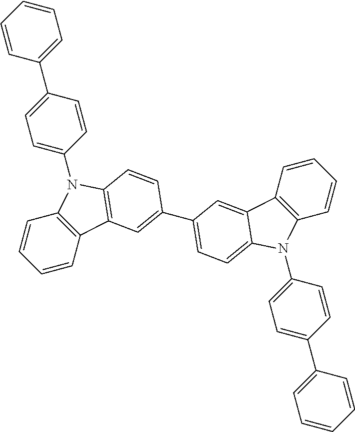 [SMILES: c1ccc(-c2ccc(-n3c4ccccc4c4cc(-c5ccc6c(c5)c5ccccc5n6-c5ccc(-c6ccccc6)cc5)ccc43)cc2)cc1] | 73% |

### <Preparation Example 7> Preparation of Compound 2-82

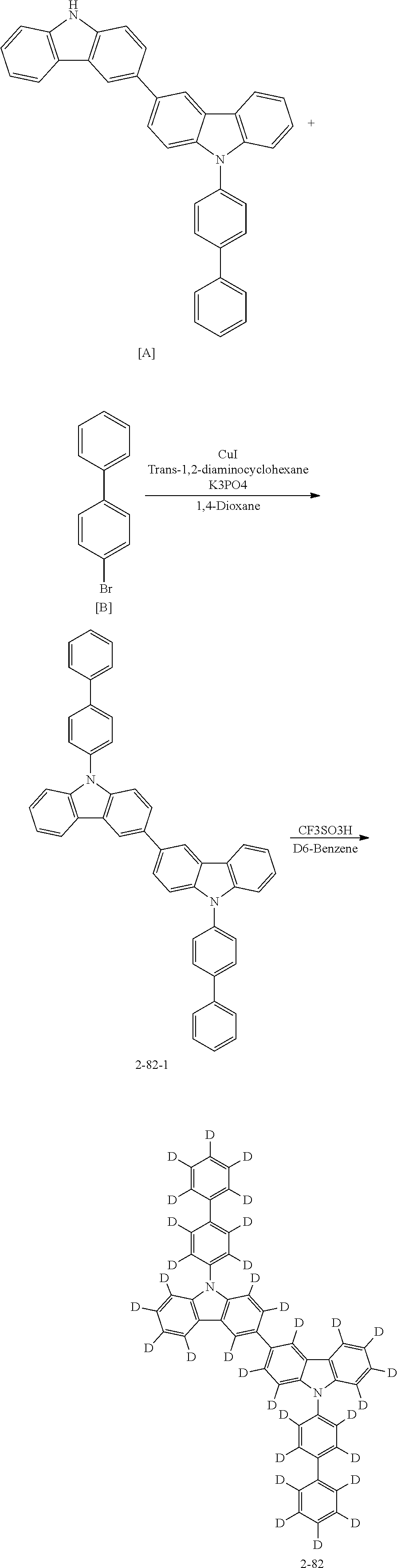

### 1) Preparation of Compound 2-82-1

In a one-neck round bottom flask, 9-([1,1′-biphenyl]-4-yl)-9H,9′H-3,3′-bicarbazole (10 g, 0.021 mol), CuI (0.40 g, 0.0021 mol), trans-1,4-diaminocyclohexane (0.024 g, 0.0021 mol) and K₃PO₄ (8.92 g, 0.042 mol) were dissolved in 1,4-oxane (100 mL), and then the mixture was refluxed for 8 hours at 125° C. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:3), and recrystallized with methanol to obtain Compound 2-82-1 (12.17 g, 91%).

### 2) Preparation of Compound 2-82

In a one-neck round bottom flask, a mixture of 3-bromo-9-phenyl-9H-carbazole (9.51 g, 0.030 mol), (9-phenyl-9H-carbazol-3-yl)boronic acid (8.61 g, 0.030 mol), Pd(PPh₃)₄ (1.73 g, 0.0015 mol), K₂CO₃ (8.29 g, 0.06 mol) and 1,4-dioxane (90 mL)/H₂O (30 mL) was refluxed at 125° C. After the reaction was completed, the result was cooled and then extracted, and the organic layer was column purified to obtain Compound 2-82 (9.89 g, yield 68%).

### <Preparation Example 8> Preparation of Compounds 2-82, 2-89, 2-92, 2-94 and 2-95

Compounds 2-82, 2-89, 2-92, 2-94 and 2-95 were synthesized in the same manner as in Preparation Example 7 except that Intermediates A′ and B′ of the following Table 4 were used instead of A and B.

TABLE 4

| Compound | A′ | B′ | Target Compound | Yield |
|---|---|---|---|---|
| 2-82 | 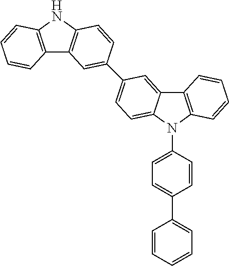 | 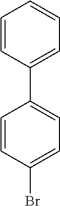 | 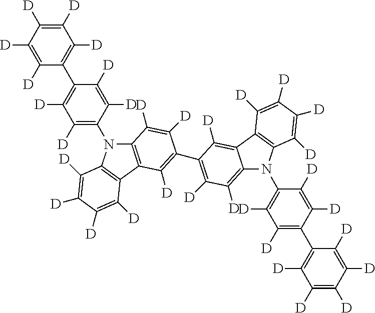 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c(-c6c([2H])c([2H])c([2H])c([2H])c6[2H])c([2H])c5[2H])c([2H])c([2H])c43)c([2H])c2[2H])c([2H])c1[2H]] | 78% |
| 2-89 | 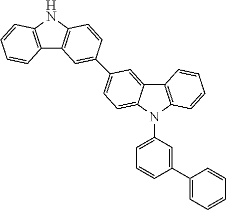 | 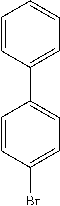 | 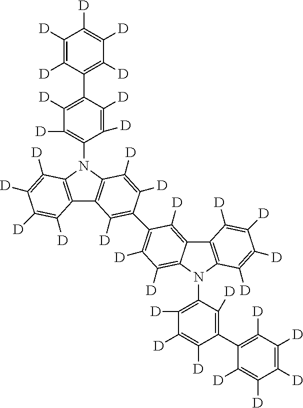 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c(-c6c([2H])c([2H])c([2H])c([2H])c6[2H])c5[2H])c([2H])c([2H])c43)c([2H])c2[2H])c([2H])c1[2H]] | 62% |
| 2-92 | 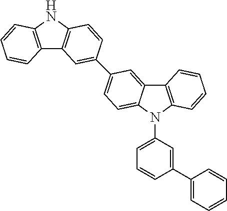 | 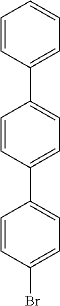 | 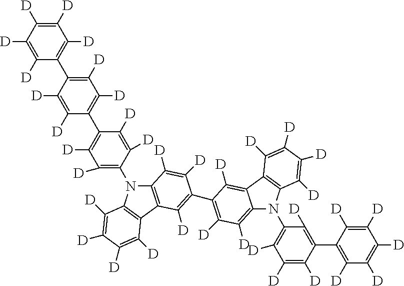 [SMILES: [2H]c1c([2H])c([2H])c(-c2c([2H])c([2H])c(-c3c([2H])c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6c([2H])c([2H])c([2H])c(-c7c([2H])c([2H])c([2H])c([2H])c7[2H])c6[2H])c([2H])c([2H])c54)c([2H])c3[2H])c([2H])c2[2H])c([2H])c1[2H]] | 77% |
| 2-94 | 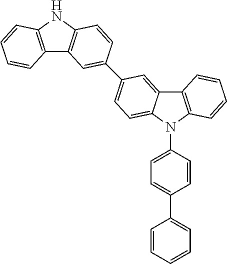 | 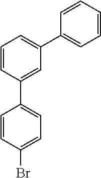 | 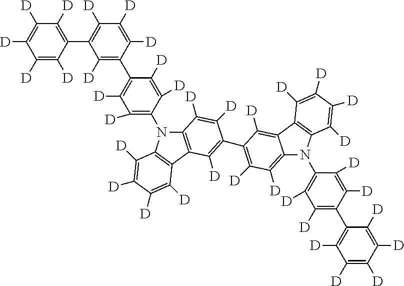 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c(-c6c([2H])c([2H])c([2H])c(-c7c([2H])c([2H])c([2H])c([2H])c7[2H])c6[2H])c([2H])c5[2H])c([2H])c([2H])c43)c([2H])c2[2H])c([2H])c1[2H]] | 83% |
| 2-95 | 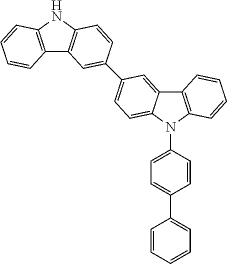 | 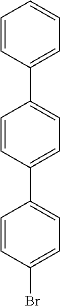 | 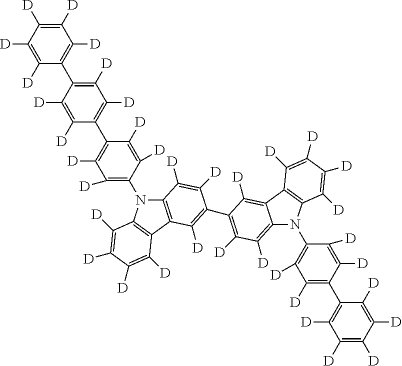 [SMILES: [2H]c1c([2H])c([2H])c(-c2c([2H])c([2H])c(-c3c([2H])c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6c([2H])c([2H])c(-c7c([2H])c([2H])c([2H])c([2H])c7[2H])c([2H])c6[2H])c([2H])c([2H])c54)c([2H])c3[2H])c([2H])c2[2H])c([2H])c1[2H]] | 62% |

### <Preparation Example 9> Preparation of Compound 3-4

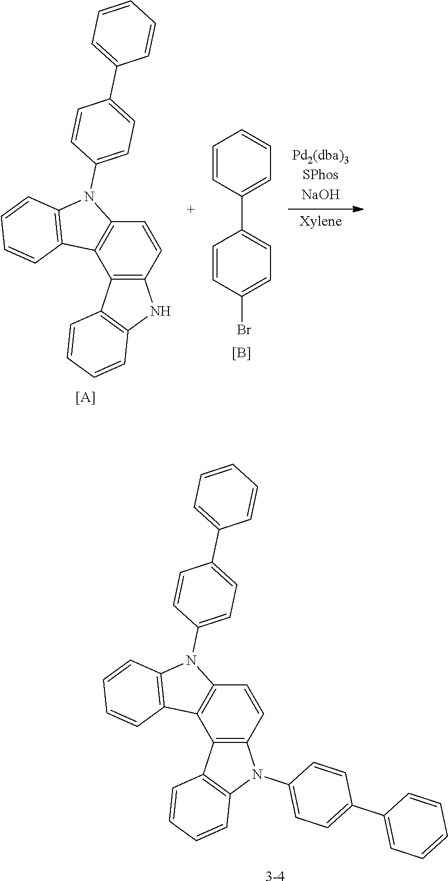

### 1) Preparation of Compound 3-4

In a one-neck round bottom flask, a mixture of 5-([1,1′-biphenyl]-4-yl)-5,8-dihydroindolo[2,3-c]carbazole (10 g, 0.024 mol), 4-bromo-1,1′-biphenyl (5.71 g, 0.024 mol), Pd₂(dba)₃ (1.10 g, 0.0012 mol), SPhos (1.97 g, 0.0048 mmol), NaOH (1.92 g, 0.048 mol) and xylene (100 mL) was refluxed at 153° C. After the reaction was completed, the result was cooled and then extracted, and the organic layer was silica gel filtered to obtain Compound 3-4 (8.48 g, yield 63%).

### <Preparation Example 10> Preparation of Compounds 3-4, 3-12, 3-26, 3-32 and 3-53

Compounds 3-4, 3-12, 3-26, 3-32 and 3-53 were synthesized in the same manner as in Preparation Example 9 except that Intermediates A′ and B′ of the following Table 5 were used instead of A and B.

TABLE 5

| Compound | A | B | Target Compound | Yield |
|---|---|---|---|---|
| 3-4 | 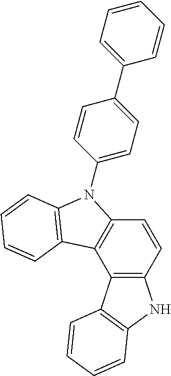 | 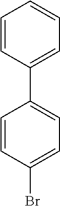 | 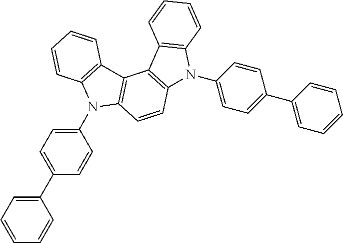 [SMILES: c1ccc(-c2ccc(-n3c4ccccc4c4c5c6ccccc6n(-c6ccc(-c7ccccc7)cc6)c5ccc43)cc2)cc1] | 63% |
| 3-12 | 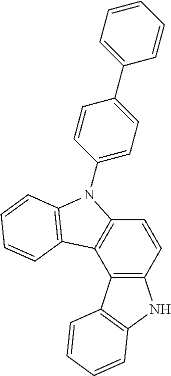 | 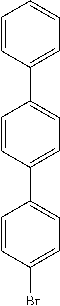 | 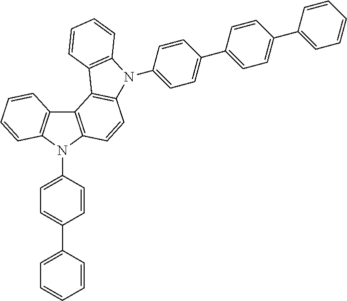 [SMILES: c1ccc(-c2ccc(-c3ccc(-n4c5ccccc5c5c6c7ccccc7n(-c7ccc(-c8ccccc8)cc7)c6ccc54)cc3)cc2)cc1] | 67% |
| 3-26 | 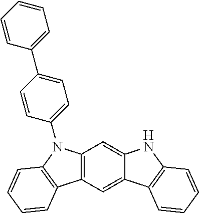 | 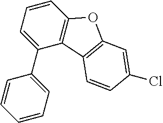 | 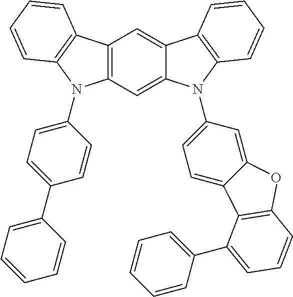 [SMILES: c1ccc(-c2ccc(-n3c4ccccc4c4cc5c6ccccc6n(-c6ccc7c(c6)oc6cccc(-c8ccccc8)c67)c5cc43)cc2)cc1] | 79% |
| 3-32 | 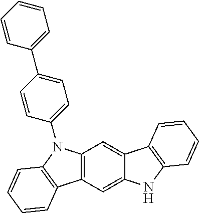 | 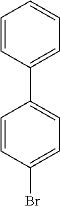 | 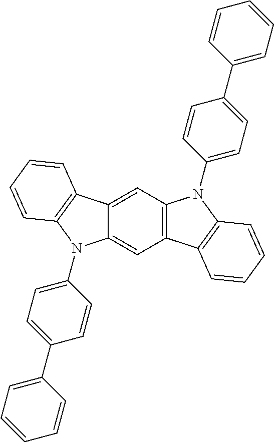 [SMILES: c1ccc(-c2ccc(-n3c4ccccc4c4cc5c(cc43)c3ccccc3n5-c3ccc(-c4ccccc4)cc3)cc2)cc1] | 84% |
| 3-53 | 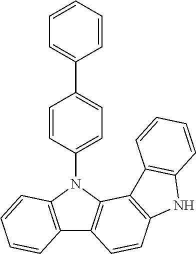 | 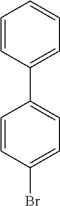 | 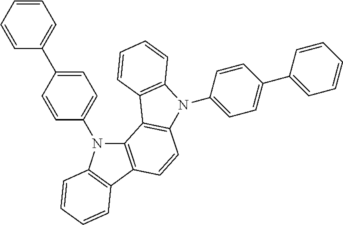 [SMILES: c1ccc(-c2ccc(-n3c4ccccc4c4c3ccc3c5ccccc5n(-c5ccc(-c6ccccc6)cc5)c34)cc2)cc1] | 60% |

### <Preparation Example 11> Preparation of Compound 3-80

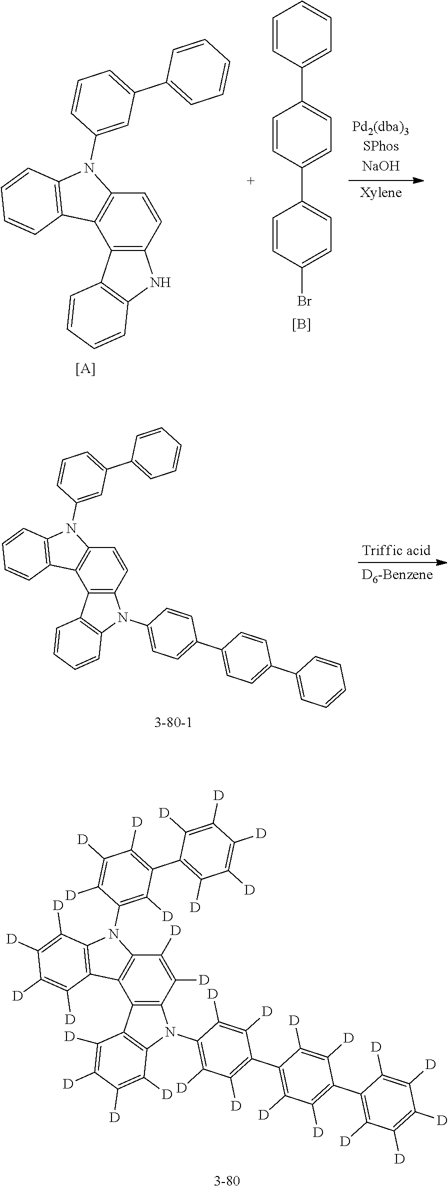

### 1) Preparation of Compound 3-80-1

In a one-neck round bottom flask, a mixture of 5-([1,1′-biphenyl]-3-yl)-5,8-dihydroindolo[2,3-c]carbazole (10 g, 0.024 mol), 4-bromo-1,1′:4′,1″-terphenyl (7.42 g, 0.024 mol), Pd₂(dba)₃ (1.10 g, 0.0012 mol), SPhos (1.97 g, 0.0048 mmol), NaOH (1.92 g, 0.048 mol) and xylene (100 mL) was refluxed at 153° C. The result was cooled and then extracted, and the organic layer was silica gel filtered to obtain Compound 3-80-1 (9.93 g, yield 65%).

### 2) Preparation of Compound 3-80

In a one-neck round bottom flask, a mixture of 5-([1,1′-biphenyl]-3-yl)-8-([1,1′:4′,1″-terphenyl]-4-yl)-5,8-dihydroindolo[2,3-c]carbazole (9.93 g, 0.016 mol), triflic acid (40.8 g, 0.27 mol) and D6-benzene (120 mL) was refluxed at 70° C. The result was quenched and extracted with dichloromethane and H₂O, then concentrated, and then silica gel filtered. After the concentration, the result was treated with methanol to obtain Compound 3-80 (6.74 g, 63%).

### <Preparation Example 12> Preparation of Compounds 3-86, 3-94, 3-112 and 3-116

Compounds 3-86, 3-94, 3-112 and 3-116 were synthesized in the same manner as in Preparation Example 11 except that Intermediates A′ and B′ of the following Table 6 were used instead of A and B.

TABLE 6

| Compound | A′ | B′ | Target Compound | Yield |
|---|---|---|---|---|
| 3-86 | 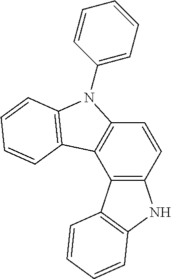 | 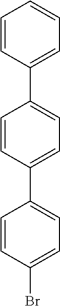 | 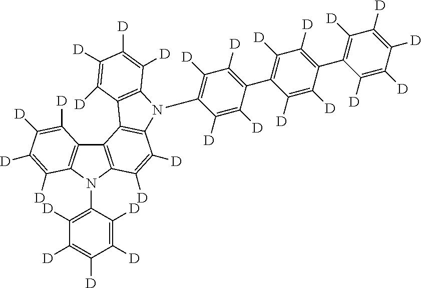 [SMILES: [2H]c1c([2H])c([2H])c(-c2c([2H])c([2H])c(-c3c([2H])c([2H])c(-n4c5c([2H])c([2H])c([2H])c([2H])c5c5c6c7c([2H])c([2H])c([2H])c([2H])c7n(-c7c([2H])c([2H])c([2H])c([2H])c7[2H])c6c([2H])c([2H])c54)c([2H])c3[2H])c([2H])c2[2H])c([2H])c1[2H]] | 67% |
| 3-94 | 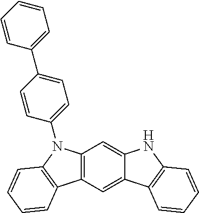 | 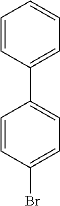 | 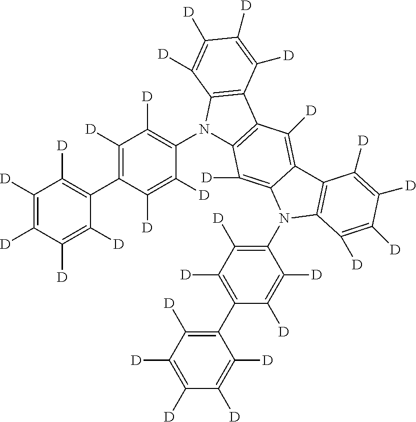 [SMILES: [2H]c1c([2H])c([2H])c(-c2c([2H])c([2H])c(-n3c4c([2H])c([2H])c([2H])c([2H])c4c4c([2H])c5c6c([2H])c([2H])c([2H])c([2H])c6n(-c6c([2H])c([2H])c(-c7c([2H])c([2H])c([2H])c([2H])c7[2H])c([2H])c6[2H])c5c([2H])c43)c([2H])c2[2H])c([2H])c1[2H]] | 79% |
| 3-112 | 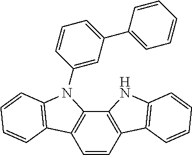 | 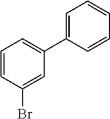 | 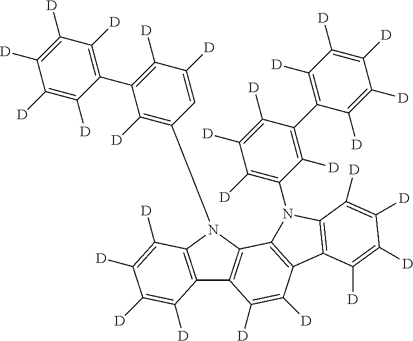 [SMILES: [2H]c1cc(-n2c3c([2H])c([2H])c([2H])c([2H])c3c3c([2H])c([2H])c4c5c([2H])c([2H])c([2H])c([2H])c5n(-c5c([2H])c([2H])c([2H])c(-c6c([2H])c([2H])c([2H])c([2H])c6[2H])c5[2H])c4c32)c([2H])c(-c2c([2H])c([2H])c([2H])c([2H])c2[2H])c1[2H]] | 84% |
| 3-116 | 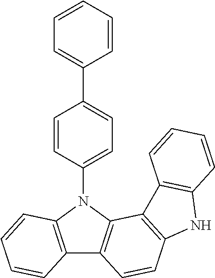 | 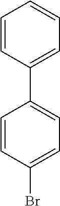 | 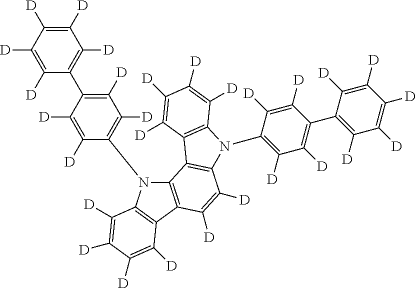 [SMILES: [2H]c1c([2H])c([2H])c(-c2c([2H])c([2H])c(-n3c4c([2H])c([2H])c([2H])c([2H])c4c4c3c([2H])c([2H])c3c5c([2H])c([2H])c([2H])c([2H])c5n(-c5c([2H])c([2H])c(-c6c([2H])c([2H])c([2H])c([2H])c6[2H])c([2H])c5[2H])c34)c([2H])c2[2H])c([2H])c1[2H]] | 60% |

The rest of compounds other than the compounds described in Preparation Examples 1 to 12 and Tables 1 to 6 were also prepared in the same manner as in the preparation examples described above, and the synthesis identification results are shown in the following Table 7 and Table 8. Table 7 shows measurement values of ¹H NMR (DMSO, 300 MHz), and Table 8 shows measurement values of FD-mass spectrometry (FD-MS: field desorption mass spectrometry).

TABLE 7

| Compound | ¹H NMR (DMSO, 300 MHz) |
|---|---|
| 1-1 | δ = 8.55 (1H, d), 8.36 (2H, m), 8.19 (1H, d), 8.03 (1H, d), 7.94 (1H, |
| | d), 7.69~7.82 (8H, m), 7.41~7.58 (13H, m), 7.16~7.25 (3H, m) |
| 1-9 | δ = 8.55 (1H, d), 8.36 (2H, m), 8.19 (1H, d), 7.94~7.98 (2H, m), |
| | 7.79~7.86 (5H, m), 7.69 (1H, d), 7.16~8.36 (18H, m) |
| 1-12 | δ = 8.55 (1H, d), 8.20~8.36 (5H, m), 8.03 (1H, d), 7.91~7.94 (4H, m), |
| | 7.68~7.79 (8H, m), 7.41~7.57 (13H, m), 7.25 (1H, d), 7.16 (1H, t) |
| 1-19 | δ = 7.96~8.03 (3H, m), 7.69~7.82 (9H, m), 7.41~7.57 (8H, m), 7.25 |
| | (2H, d) |
| 1-24 | δ = 8.45 (1H, d), 8.30~8.36 (3H, m), 7.93~8.01 (3H, m), 7.79 (2H, d), |
| | 7.70 (1H, s), 7.41~7.56 (9H, m) |
| 1-25 | δ = 8.55 (1H, d), 8.36 (2H, m), 8.19 (1H, d), 8.03 (1H, d), 7.94 (1H, |
| | d), 7.35~7.82 (22H, m), 7.16~7.20 (2H, m) |
| 1-28 | δ = 8.55 (1H, d), 8.36 (2H, m), 8.19 (1H, d), 8.02~8.08 (3H, m), 7.94 |
| | (1H, d), 7.69~7.82 (5H, m), 7.31~7.58 (15H, m), 7.16~7.20 (2H, t) |
| 1-30 | δ = 8.55 (1H, d), 8.31~8.36 (3H, m), 8.03 (1H, d), 7.91~7.94 (2H, m), |
| | 7.69~7.82 (12H, m), 7.31~7.61 (14H, m), 7.16 (1H, t) |
| 1-34 | δ = 8.55 (1H, d), 8.36 (2H, m), 8.19 (1H, d), 8.03 (1H, d), 7.94 (1H, |
| | d), 7.69~7.82 (9H, m), 7.16~7.61 (19H, m) |
| 1-36 | δ = 8.55 (1H, d), 8.19 (1H, d), 7.94~8.03 (4H, m), 7.16~7.82 (28H, m) |
| 1-39 | δ = 8.55 (1H, d), 8.36 (2H, m), 8.19 (1H, d), 8.03 (1H, d), 7.94 (1H, |
| | d), 7.69~7.82 (9H, m), 7.31~7.61 (13H, m), 7.16~7.20 (2H, t) |
| 1-42 | δ = 8.55 (1H, d), 8.36 (2H, m), 8.19 (1H, d), 7.94~7.98 (2H, m), |
| | 7.69~7.86 (7H, m), 7.31~7.61 (15H, m), 7.16~7.20 (2H, t) |
| 1-46 | δ = 8.55 (1H, d), 8.36 (2H, m), 8.12~8.24 (5H, m), 7.94~7.99 (3H, m), |
| | 7.69~7.82 (5H, m), 7.31~7.61 (12H, m), 7.16 (1H, t) |
| 1-50 | δ = 8.55 (1H, d), 8.20~8.36 (5H, m), 8.03 (1H, d), 7.91~7.94 (4H, m), |
| | 7.31~7.82 (22H, m), 7.16 (1H, t) |
| 1-52 | δ = 8.55 (2H, d), 8.20~8.36 (6H, m), 8.06 (1H, d), 7.94 (2H, d), 7.82 |
| | (1H, d), 7.69~7.74 (3H, m), 7.31~7.61 (14H, m), 7.16~7.19 (5H, m) |
| 1-56 | δ = 8.55 (1H, d), 8.19~8.24 (3H, m), 7.94~8.03 (6H, m), 7.16~7.82 |
| | (24H, m) |
| 1-58 | δ = 8.55 (1H, d), 8.36 (2H, m), 8.19 (1H, d), 8.12 (1H, s), 7.92~8.03 |
| | (5H, m), 7.31~7.82 (18H, m), 7.16~7.20 (2H, m) |
| 1-60 | δ = 8.55 (1H, d), 8.36 (2H, m), 8.17~8.24 (3H, m), 7.89~8.03 (5H, m), |
| | 7.31~7.82 (22H, m), 7.16 (1H, t) |
| 1-66 | δ = 8.55 (1H, d), 8.36 (2H, m), 8.03 (1H, d), 7.69~7.82 (6H, m), |
| | 7.43~7.57 (9H, m), 7.25 (1H, t) |
| 1-67 | δ = 8.55 (1H, d), 8.36 (2H, m), 8.03 (1H, d), 7.69~7.84 (7H, m), |
| | 7.41~7.57 (8H, m), 7.25 (5H, m) |
| 1-69 | δ = 8.55 (1H, d), 8.36 (2H, m), 8.08 (1H, d), 7.79~7.88 (5H, m), 7.69 |
| | (1H, d), 7.41~7.51 (8H, m), 7.25 (1H, t) |
| 1-72 | δ = 8.55 (1H, d), 7.36 (2H, m), 7.98 (1H, d), 7.79~7.86 (4H, m), |
| | 7.25~7.54 (11H, m) |
| 1-74 | δ = 8.55 (1H, d), 8.36 (2H, m), 8.17~8.24 (5H, m), 7.94 (1H, d), 7.84 |
| | (1H, s), 7.75 (2H, d), 7.41~7.52 (8H, m), 7.25 (1H, t) |
| 1-76 | δ = 8.55 (1H, d), 8.36 (2H, m), 8.12 (1H, s), 7.92~8.03 (4H, m), |
| | 7.79~7.84 (3H, m), 7.68 (1H, t), 7.41~7.50 (7H, m), 7.25 (1H, t) |
| 1-78 | δ = 8.55 (1H, d), 8.45 (1H, d), 8.30~8.36 (3H, m), 8.01 (1H, s), 7.93 |
| | (1H, d), 7.79 (2H, d), 7.41~7.50 (10H, m), 7.25 (1H, t) |
| 1-81 | δ = 8.55 (1H, d), 8.36 (2H, m), 8.19 (1H, d), 7.94~8.03 (3H, m), |
| | 7.69~7.82 (8H, m), 7.35~7.58 (12H, m), 7.16~7.25 (3H, m) |
| 1-85 | δ = 8.55 (1H, d), 8.36 (2H, m), 7.69~7.98 (16H, m), 7.35~7.57 (13H, m), |
| | 7.25 (1H, d), 7.16 (1H, t) |
| 1-87 | δ = 8.55 (1H, d), 8.36 (2H, m), 7.91~8.03 (5H, m), 7.68~7.82 (11H, m), |
| | 7.35~7.57 (13H, m), 7.25 (1H, d), 7.16 (1H, t) |
| 1-90 | δ = 8.55 (1H, d), 8.36 (2H, m), 8.19 (1H, d), 7.94~8.03 (3H, m), |
| | 7.69~7.82 (8H, m), 7.35~7.58 (12H, m), 7.16~7.25 (7H, m) |
| 1-92 | δ = 8.55 (1H, d), 8.19 (1H, d), 7.94~8.03 (5H, m), 7.69~7.82 (10H, m), |
| | 7.35~7.58 (12H, m), 7.16~7.25 (5H, m) |
| 1-95 | δ = 8.55 (1H, d), 8.36 (2H, m), 8.19 (1H, d), 7.94~8.03 (3H, m), |
| | 7.69~7.82 (8H, m), 7.35~7.58 (12H, m), 7.16~7.25 (3H, m) |
| 1-98 | δ = 8.55 (1H, d), 8.36 (2H, m), 8.19 (1H, d), 7. 94~7.98 (3H, m), |
| | 7.79~7.86 (5H, m), 7.69 (1H, d), 7.16~7.58 (17H, m) |
| 1-100 | δ = 8.55 (1H, d), 8.19 (1H, d), 7.94~7.98 (5H, m), 7.69~7.86 (8H, m), |
| | 7.16~7.58 (19H, m) |
| 1-103 | δ = 8.55 (1H, d), 8.36 (2H, m), 7.17~8.24 (6H, m), 7.94~7.98 (3H, m), |
| | 7.69~7.82 (4H, m), 7.35~7.58 (11H, m), 7.16~7.25 (3H, m) |
| 1-106 | δ = 8.55 (1H, d), 8.20~8.36 (5H, m), 7.91~8.03 (6H, m), 7.68~7.82 (8H, |
| | m), 7.35~7.57 (12H, m), 7.25 (1H, d), 7.16 (1H, t) |
| 1-109 | δ = 8.55 (1H, d), 8.36 (2H, m), 8.19~8.24 (3H, m), 7.94~8.03 (6H, m), |
| | 7.35~7.82 (18H, m), 7.16~7.25 (3H, m) |
| 1-115 | δ = 8.55 (1H, d), 8.36 (2H, m), 8.17~8.24 (4H, m), 7.94~8.03 (4H, m), |
| | 7.68~7.82 (5H, m), 7.35~7.58 (11H, m), 7.16~7.25 (3H, m) |
| 1-120 | δ = 8.55 (1H, d), 8.45 (1H, d), 8.30 (1H, s), 8.19 (1H, d), 7.93~8.01 |
| | (6H, m), 7.69~7.82 (6H, m), 7.41~7.58 (13H, m), 7.16~7.25 (5H, m) |
| 1-122 | δ = 8.36 (2H, m), 7.98~8.03 (2H, m), 7.75~7.88 (8H, m), 7.41~7.50 |
| | (7H, m) |
| 1-127 | δ = 8.36 (2H, m), 8.08 (1H, d), 7.77~7.88 (7H, m), 7.69~7.70 (2H, m), |
| | 7.41~7.57 (8H, m) |
| 1-131 | δ = 8.38 (1H, d), 7.70~7.88 (14H, m), 7.41~7.61 (8H, m) |
| 1-134 | δ = 8.36 (2H, m), 8.17~8.24 (5H, m), 7.94 (1H, d), 7.86 (1H, s), |
| | 7.75~7.81 (3H, m), 7.41~7.50 (6H, m) |
| 1-136 | δ = 8.45 (1H, d), 8.30~8.36 (3H, m), 8.00~8.01 (2H, s), 7.93 (1H, d), |
| | 7.86 (1H, s), 7.79 (2H, d), 7.70 (1H, s), 7.41~7.56 (10H, m) |
| 1-137 | δ = 8.55 (1H, d), 8.36 (2H, m), 8.19 (1H, d), 8.03 (1H, d), 7.94 (2H, |
| | d), 7.69~7.82 (8H, m), 7.35~7.58 (13H, m), 7.16~7.20 (2H, t) |
| 1-144 | δ = 8.55 (1H, d), 8.29~8.36 (3H, m), 8.02~8.08 (4H, m), 7.94 (2H, d), |
| | 7.76~7.82 (3H, m), 7.69 (1H, d), 7.31~7.57 (15H, m), 7.16~7.19 (5H, m) |
| 1-148 | δ = 8.55 (1H, d), 8.19 (1H, d), 7.94~8.03 (5H, m), 7.69~7.82 (10H, m), |
| | 7.16~7.58 (17H, m) |
| 1-154 | δ = 8.55 (1H, d), 8.36 (2H, m), 8.19 (1H, d), 7.94~7.98 (3H, m), |
| | 7.79~7.86 (5H, m), 7.69 (1H, d), 7.31~7.58 (15H, m), 7.16~7.20 (2H, t) |
| 1-158 | δ = 8.55 (1H, d), 8.36 (2H, m), 8.12~8.24 (5H, m), 7.94~7.99 (4H, m), |
| | 7.69~7.82 (4H, m), 7.31~7.58 (12H, m), 7.16~7.20 (2H, t) |
| 1-161 | δ = 8.55 (1H, d), 8.36 (2H, m), 8.20~8.24 (2H, m), 7.89~7.94 (7H, m), |
| | 7.68~7.82 (8H, m), 7.31~7.57 (13H, m), 7.16 (1H, t) |
| 1-166 | δ = 8.55 (1H, d), 8.36 (2H, m), 8.19~8.24 (3H, m), 8.03 (1h, d), 7.94 |
| | (4H, m), 7.69~7.82 (5H, m), 7.16~7.57 (18H, m) |
| 1-176 | δ = 8.55 (1H, d), 8.45 (1H, d), 8.30 (1H, s), 8.19 (1H, d), 7.93~8.01 |
| | (6H, m), 7.69~7.82 (6H, m), 7.16~7.58 (23H, m) |
| 1-178 | δ = 8.36 (2H, m), 8.03 (1H, d), 7.75~7.88 (8H, m), 7.41~7.50 (6H, m) |
| 1-180 | δ = 8.36 (2H, m), 8.03 (1H, d), 7.68~7.82 (8H, m), 7.41~7.57 (9H, m) |
| 1-186 | δ = 7.96~7.98 (3H, m), 7.68~7.86 (8H, m), 7.25~7.54 (11H, m) |
| 1-189 | δ = 8.36 (2H, m), 8.20~8.24 (2H, m), 8.03 (1H, d), 7.94 (2H, d), |
| | 7.68~7.81 (5H, m), 7.41~7.50 (6H, m), 7.25 (4H, s) |
| 1-191 | δ = 8.36 (2H, m), 8.17~8.24 (3H, m), 8.03 (1H, d), 7.94 (1H, d), |
| | 7.68~7.75 (4H, m), 7.41~7.50 (6H, m) |
| 1-193 | δ = 8.55 (1H, d), 8.36 (2H, m), 8.19 (1H, d), 8.03 (2H, d), 7.94 (1H, |
| | d), 7.69~7.82 (8H, m), 7.35~7.58 (12H, m), 7.16~7.25 (3H, m) |
| 1-195 | δ = 8.55 (1H, d), 8.36 (2H, m), 8.19 (1H, d), 8.03 (3H, d), 7.94 (1H, |
| | d), 7.75~7.82 (8H, m), 7.35~7.58 (11H, m), 7.16~7.25 (3H, m) |
| 1-197 | δ = 8.55 (1H, d), 8.36 (2H, m), 7.69~8.03 (16H, m), 7.35~7.57 (13H, m), |
| | 7.25 (1H, d), 7.16 (1H, t) |
| 1-198 | δ = 8.55 (1H, d), 8.31~8.36 (3H, m), 8.03 (2H, d), 7.91~7.94 (2H, m), |
| | 7.74~7.82 (11H, m), 7.35~7.57 (13H, m), 7.25 (1H, d), 7.16 (1H, t) |
| 1-201 | δ = 8.55 (1H, d), 8.36 (2H, m), 8.19 (1H, d), 8.03 (2H, d), 7.94 (2H, |
| | m), 7.69~7.82 (9H, m), 7.35~7.61 (14H, m), 7.16~7.20 (3H, m) |
| 1-205 | δ = 8.55 (1H, d), 8.36 (2H, m), 8.19 (1H, d), 8.03~8.08 (2H, m), |
| | 7.76~7.82 (7H, m), 7.69 (1H, d), 7.35~7.57 (13H, m), 7.16~7.25 (3H, m) |
| 1-208 | δ = 8.55 (1H, d), 8.36 (2H, m), 7.89~8.03 (5H, m), 7.69~7.82 (11H, m), |
| | 7.35~7.57 (13H, m), 7.25 (1H, d), 7.16 (1H, t) |
| 1-209 | δ = 8.55 (1H, d), 8.38 (1H, d), 8.19 (1H, d), 8.03 (1H, d), 7.69~7.94 |
| | (14H, m), 7.35~7.61 (13H, m), 7.16~7.25 (3H, m) |
| 1-210 | δ = 8.55 (1H, d), 8.36 (2H, m), 8.19 (1H, d), 7.94~8.03 (3H, m), |
| | 7.76~7.86 (6H, m), 7.16~7.58 (17H, m) |
| 1-213 | δ = 8.55 (1H, d), 8.36 (2H, m), 8.19~8.24 (3H, m), 8.03 (2H, d), 7.94 |
| | (3H, d), 7.76~7.82 (4H, m), 7.68 (1H, t), 7.35~7.58 (11H, m), |
| | 7.16~7.25 (3H, m) |
| 1-216 | δ = 8.55 (2H, d), 8.32~8.36 (3H, m), 8.19~8.24 (3H, m), 8.03 (1H, d), |
| | 7.94 (2H, d), 7.70~7.82 (3H, m), 7.35~7.58 (13H, m), 7.16~7.25 (3H, m) |
| 1-217 | δ = 8.55 (1H, d), 8.36 (2H, m), 8.20~8.24 (2H, m), 7.89~8.03 (7H, m), |
| | 7.68~7.82 (8H, m), 7.417.55 (12H, m), 7.25 (1H, d), 7.16 (1H, t) |
| 1-219 | δ = 8.55 (1H, d), 8.36 (2H, m), 8.17~8.24 (5H, m), 8.03 (1H, d), |
| | 7.91~7.94 (3H, m), 7.75~8.03 (7H, m), 7.35~7.56 (13H, m), 7.25 (1H, |
| | d), 7.16 (1H, t) |
| 1-222 | δ = 8.55 (1H, d), 8.36 (2H, m), 8.19~8.24 (3H, m), 8.03 (2H, d), 7.94 |
| | (3H, d), 7.68~7.82 (5H, m), 7.35~8.03 (11H, m), 7.16~7.25 (7H, m) |
| 1-224 | δ = 8.55 (1H, d), 8.19~8.24 (3H, m), 7.94~8.03 (7H, m), 7.68~7.82 |
| | (7H, m), 7.35~7.58 (11H, m), 7.16~7.25 (5H, m) |
| 1-226 | δ = 8.55 (1H, d), 8.36 (2H, m), 8.19 (1H, d), 8.12 (1H, s), 7.92~8.03 |
| | (6H, m), 7.76~7.82 (4H, m), 7.68 (1H, t), 7.35~7.58 (11H, m), |
| | 7.16~7.25 (3H, m) |
| 1-227 | δ = 8.55 (1H, d), 8.36 (2H, m), 8.17~8.24 (4H, m), 8.03 (2H, d), 7.94 |
| | (2H, d), 7.68~7.82 (5H, m), 7.35~7.58 (11H, m), 7.16~7.20 (2H, m) |
| 1-232 | δ = 8.55 (1H, d), 8.45 (1H, d), 8.30 (1H, s), 8.19 (1H, d), 7.93~8.03 |
| | (6H, m), 7.75~7.82 (6H, m), 7.35~7.58 (13H, m), 7.16~7.25 (5H, m) |
| 1-233 | δ = 8.36 (2H, m), 8.03 (1H, d), 7.91~7.94 (2H, m), 7.69~7.82 (7H, m), |
| | 7.41~7.57 (8H, m) |
| 1-236 | δ = 8.36 (2H, m), 8.02~8.08 (3H, m), 7.91~7.94 (2H, m), 7.76~7.82 (2H, |
| | m), 7.41~7.51 (10H, m) |
| 1-238 | δ = 8.36 (2H, m), 8.03 (2H, d), 7.69~7.82 (9H, m), 7.41~7.57 (8H, m) |
| 1-241 | δ = 8.36 (2H, m), 8.08 (1H, d), 7.79~7.94 (6H, m), 7.69 (1H, d), |
| | 7.41~7.57 (11H, m) |
| 1-245 | δ = 8.36 (2H, m), 8.20~8.24 (2H, m), 8.12 (2H, m), 7.91~7.99 (4H, m), |
| | 7.75 (2H, d), 7.41~7.50 (7H, m) |
| 1-247 | δ = 8.36 (2H, m), 8.17~8.24 (3H, m), 8.03 (1H, d), 7.91~7.94 (3H, m), |
| | 7.68~7.75 (3H, m), 7.41~7.50 (7H, m) |
| 1-248 | δ = 8.55 (1H, d), 8.36 (2H, m), 8.03 (1H, d), 7.91~7.94 (4H, m), |
| | 7.68~7.79 (4H, m), 7.41~7.50 (7H, m) |
| 1-249 | δ = 8.55 (1H, d), 8.36 (2H, m), 8.19 (1H, d), 8.03 (2H, d), 7.94 (1H, |
| | d), 7.69~7.82 (9H, m), 7.31~7.58 (12H, m), 7.16~7.20 (2H, t) |
| 1-251 | δ = 8.36 (2H, m), 8.19 (1H, d), 8.03 (3H, d), 7.94 (1H, d), 7.74~7.82 |
| | (9H, m), 7.31~7.61 (11H, m), 7.16~7.20 (2H, t) |
| 1-253 | δ = 8.55 (1H, d), 8.36 (2H, m), 7.74~8.03 (17H, m), 7.31~7.61 (13H, |
| | m), 7.16 (1H, t) |
| 1-256 | δ = 8.55 (1H, d), 8.29~8.36 (3H, m), 8.02~8.08 (5H, m), 7.94 (1H, d), |
| | 7.74~7.82 (5H, m), 7.61 (1H, s), 7.31~7.51 (13H, m), 7.16~7.19 (5H, m) |
| 1-257 | δ = 8.55 (1H, d), 8.36 (2H, m), 8.19 (1H, d), 8.03 (2H, d), 7.94 (2H, |
| | m), 7.31~7.82 (24H, m) 7.16~7.20 (2H, t) |
| 1-260 | δ = 8.55 (1H, d), 8.19 (1H, d), 7.94~8.03 (5H, m), 7.69~7.82 (11H, m), |
| | 7.16~7.58 (16H, m) |
| 1-263 | δ = 8.55 (1H, d), 8.36 (2H, m), 8.19 (1H, d), 8.03 (2H, d), 7.94 (1H, |
| | d), 7.69~7.82 (9H, m), 7.31~7.61 (12H, m), 7.16~7.20 (2H, t) |
| 1-266 | δ = 8.55 (1H, d), 8.36 (2H, m), 8.19 (1H, d), 7.94~8.03 (3H, m), |
| | 7.74~7.86 (7H, m), 7.31~7.58 (14H, m), 7.16~7.20 (2H, t) |
| 1-270 | δ = 8.55 (1H, d), 8.36 (2H, m), 8.12~8.24 (5H, m), 7.94~8.03 (4H, m), |
| | 7.74~7.82 (5H, m), 7.31~7.61 (11H, m), 7.16~7.20 (2H, t) |
| 1-273 | δ = 8.55 (1H, d), 8.36 (2H, m), 8.20~8.24 (2H, m), 7.89~8.03 (7H, m), |
| | 7.61~7.82 (10H, m), 7.31~7.50 (11H, m), 7.16 (1H, t) |
| 1-274 | δ = 8.55 (1H, d), 8.20~8.36 (5H, m), 8.06 (2H, d), 7.91~7.94 (4H, m), |
| | 7.61~7.82 (10H, m), 7.31~7.50 (11H, m), 7.16 (1H, t) |
| 1-276 | δ = 8.55 (1H, d), 8.20~8.36 (6H, m), 8.03~8.06 (2H, m), 7.94 (2H, d), |
| | 7.70~7.82 (4H, m), 7.61 (1H, s), 7.31~7.51 (12H, m), 7.16~7.19 (5H, m) |
| 1-279 | δ = 8.55 (1H, d), 8.36 (2H, m), 8.12~8.24 (5H, m), 7.94~8.03 (5H, m), |
| | 7.74~7.82 (6H, m), 7.31~7.61 (13H, m), 7.16~7.20 (2H, t) |
| 1-280 | δ = 8.55 (1H, d), 8.20~8.24 (2H, m), 7.94~8.03 (7H, m), 7.25~7.82 |
| | (23H, m) |
| 1-287 | δ = 8.55 (1H, d), 8.45 (1H, d), 8.30~8.36 (3H, m), 7.89~8.03 (6H, m), |
| | 7.74~7.82 (8H, m), 7.31~7.61 (14H, m), 7.16 (1H, t) |
| 1-289 | δ = 8.36 (2H, m), 8.03 (1H, d), 7.69~7.82 (7H, m), 7.41~7.57 (8H, m) |
| 1-296 | δ = 8.36 (2H, m), 8.08 (1H, d), 7.97 (1H, s), 7.79~7.88 (6H, m), 7.69 |
| | (1H, d), 7.41~7.57 (8H, m) |
| 1-297 | δ = 8.36 (2H, m), 8.20~8.24 (2H, m), 8.03 (1H, d), 7.94 (2H, d), |
| | 7.79~7.84 (3H, m), 7.68 (1H, t), 7.41~7.52 (8H, m) |
| 1-300 | δ = 8.36 (2H, m), 8.12 (1H, s), 7.92~8.03 (5H, m), 7.79 (2H, d), 7.68 |
| | (1H, t), 7.41~7.50 (6H, m) |
| 1-301 | δ = 8.55 (1H, d), 8.36 (2H, m), 8.19 (1H, d), 7.94~8.03 (4H, m), |
| | 7.76~7.82 (8H, m), 7.35~7.58 (11H, m), 7.16~7.25 (3H, m) |
| 1-302 | δ = 8.55 (1H, d), 8.36 (2H, m), 8.19 (1H, s), 7.75~8.03 (13H, m), |
| | 7.35~7.58 (9H, m), 7.16~7.25 (3H, m) |
| 1-305 | δ = 8.55 (1H, d), 8.36 (2H, m), 7.69~8.03 (17H, m), 7.41~7.57 (12H, m), |
| | 7.25 (1H, d), 7.16 (1H, t) |
| 1-310 | δ = 8.55 (1H, d), 8.36 (2H, m), 8.19 (1H, d), 7.94~8.03 (4H, m), |
| | 7.69~7.82 (8H, m), 7.35~7.58 (11H, m), 7.16~7.25 (7H, m) |
| 1-312 | δ = 8.55 (1H, d), 8.19 (1H, d), 7.94~8.03 (6H, m), 7.69~7.82 (10H, m), |
| | 7.35~7.58 (11H, m), 7.16~7.25 (5H, m) |
| 1-313 | δ = 8.55 (1H, d), 8.36 (2H, m), 8.19 (1H, d), 7.76~8.08 (10H, m), 7.69 |
| | (1H, d), 7.41~7.58 (12H, m), 7.16~7.25 (3H, m) |
| 1-316 | δ = 8.55 (1H, d), 8.36 (2H, m), 7.89~8.03 (6H, m), 7.75~7.82 (11H, m), |
| | 7.35~7.57 (12H, m), 7.25 (1H, d), 7.16 (1H, t) |
| 1-320 | δ = 8.55 (1H, d), 8.19 (1H, d), 7.94~7.98 (6H, m), 7.75~7.86 (8H, m), |
| | 7.16~7.58 (18H, m) |
| 1-321 | δ = 8.55 (1H, d), 8.36 (2H, m), 8.20~8.24 (2H, m), 7.94~8.03 (6H, m), |
| | 7.68~7.82 (5H, m), 7.35~7.58 (10H, m), 7.16~7.25 (3H, m) |
| 1-324 | δ = 8.55 (2H, d), 8.32~8.36 (3H, m), 8.19~8.24 (3H, m), 7.94~8.03 (4H, |
| | m), 7.70~7.82 (3H, m), 7.35~7.58 (12H, m), 7.16~7.25 (3H, m) |
| 1-326 | δ = 8.55 (1H, d), 8.24~8.36 (5H, m), 7.91~8.03 (7H, m), 7.68~7.82 (8H, |
| | m), 7.35~7.50 (11H, m), 7.25 (1H, d), 7.16 (1H, t) |
| 1-330 | δ = 8.55 (1H, d), 8.36 (2H, m), 8.19~8.24 (3H, m), 7.94~8.03 (6H, m), |
| | 7.68~7.82 (5H, m), 7.35~7.58 (10H, m), 7.16~7.25 (7H, m) |
| 1-333 | δ = 8.55 (1H, d), 8.36 (2H, m), 8.19 (1H, d), 7.94~8.03 (5H, m), |
| | 7.76~7.82 (5H, m), 7.35~7.54 (10H, m), 7.16~7.25 (2H, m) |
| 1-337 | δ = 8.55 (1H, d), 8.38 (1H, d), 8.19 (1H, d), 8.12 (1H, s), 7.92~8.03 |
| | (8H, m), 7.41~7.75 (19H, m), 7.16~7.25 (3H, m) |
| 1-341 | δ = 8.36 (2H, m), 7.99~8.03 (2H, m), 7.69~7.82 (8H, m), 7.41~7.57 |
| | (7H, m) |
| 1-347 | δ = 8.36 (2H, m), 7.98~7.99 (2H, m), 7.77~7.86 (5H, m), 7.31~7.54 |
| | (13H, m) |
| 1-351 | δ = 8.36 (2H, m), 8.20~8.24 (2H, m), 7.94~8.03 (6H, m), 7.61~7.75 |
| | (7H, m), 7.41~7.50 (7H, m) |
| 1-352 | δ = 8.36 (2H, m), 8.20~8.24 (2H, m), 8.12 (2H, m), 7.94~7.99 (4H, m), |
| | 7.73~7.77 (4H, m), 7.61 (2H, d), 7.41~7.50 (6H, m) |
| 1-355 | δ = 8.55 (1H, d), 8.36 (2H, m), 8.19 (1H, d), 8.03 (3H, d), 7.94 (2H, |
| | d), 7.75~7.82 (8H, m), 7.31~7.50 (11H, m), 7.16~7.20 (2H, t) |
| 1-356 | δ = 8.55 (1H, d), 8.36 (2H, m), 8.19 (1H, d), 8.02~8.08 (4H, m), 7.94 |
| | (2H, d), 7.76~7.82 (4H, m), 7.31~7.58 (14H, m), 7.16~7.20 (2H, t) |
| 1-360 | δ = 8.55 (1H, d), 8.29~8.36 (3H, m), 8.02~8.08 (5H, m), 7.94 (2H, d), |
| | 7.76~7.82 (4H, m), 7.31~7.51 (14H, m), 7.16~7.19 (5H, m) |
| 1-364 | δ = 8.55 (1H, d), 8.19 (1H, d), 7.94~8.03 (6H, m), 7.69~7.82 (10H, m), |
| | 7.16~7.58 (16H, m) |
| 1-368 | δ = 8.55 (1H, d), 8.36 (2H, m), 7.89~8.03 (6H, m), 7.75~7.82 (11H, m), |
| | 7.31~7.57 (13H, m), 7.16 (1H, t) |
| 1-372 | δ = 8.55 (1H, d), 8.19 (1H, d), 7.75~8.03 (14H, m), 7.16~7.58 (18H, m) |
| 1-373 | δ = 8.55 (1H, d), 8.36 (2H, m), 8.19~8.24 (3H, m), 7.94~8.03 (6H, m), |
| | 7.76~7.82 (4H, m), 7.68 (1H, t), 7.35~7.58 (11H, m), 7.16~7.20 (2H, t) |
| 1-379 | δ = 8.35 (1H, d), 8.36 (2H, m), 8.17~8.24 (5H, m), 8.03 (1H, d), |
| | 7.91~7.94 (4H, m), 7.68~7.82 (7H, m), 7.31~7.50 (13H, m), 7.16 (1H, t) |
| 1-381 | δ = 8.55 (1H, d), 8.36 (2H, m), 8.19~8.24 (3H, m), 7.94~8.03 (7H, m), |
| | 7.31~7.75 (19H, m), 7.16~7.20 (2H, t) |
| 1-388 | δ = 8.55 (1H, d), 8.36 (2H, m), 8.17~8.24 (3H, m), 7.89~8.03 (7H, m), |
| | 7.75~7.82 (8H, m), 7.31~7.50 (12H, m), 7.16 (1H, t) |
| 1-395 | δ = 8.36 (2H, m), 8.03 (2H, d), 7.75~7.82 (6H, m), 7.41~7.54 (7H, m) |
| 1-400 | δ = 8.36 (2H, m), 8.03 (1H, d), 7.94 (1H, d), 7.69~7.82 (7H, m), |
| | 7.41~7.57 (8H, m) |
| 1-402 | δ = 8.36 (2H, m), 8.20~8.24 (2H, m), 8.03 (1H, d), 7.94 (2H, d), |
| | 7.79~7.82 (4H, m), 7.68 (1H, t), 7.41~7.54 (7H, m) |
| 1-408 | δ = 8.45 (1H, d), 8.30~8.36 (3H, m), 7.93~8.01 (3H, m), 7.79~7.81 (3H, |
| | m), 7.41~7.56 (8H, m) |
| 2-1 | δ = 8.55 (1H, d), 8.30 (1H, d), 8.13~8.19 (2H, m), 7.89~7.99 (4H, m), |
| | 7.77 (1H, d), 7.50~7.62 (12H, m), 7.35 (1H, t), 7.16~7.20 (2H, t) |
| 2-4 | δ = 8.55 (1H, d), 8.12~8.18 (2H, m), 7.84~8.00 (3H, m), 7.77~7.79 (4H, |
| | m), 7.25~7.68 (22H, m) |
| 2-16 | δ = 9.05 (1H, s), 8.55 (1H, d), 8.19~8.33 (7H, m), 7.89~7.99 (5H, m), |
| | 7.50~7.77 (13H, m), 7.35 (1H, t), 7.16~7.20 (2H, t) |
| 2-27 | δ = 8.55 (1H, m), 8.09~8.18 (4H, m), 7.94~8.00 (2H, m), 7.77 (2H, m) |
| 2-28 | δ = 8.55 (1H, m), 8.09~8.18 (3H, m), 8.00~8.79 (2H, m), 7.87 (1H, m), |
| | 7.77~7.79 (4H, m), 7.63~7.69 (4H, m), 7.25~7.52 (12H, m) |
| 2-32 | δ = 8.55 (1H, m), 8.12~8.18 (2H, m), 7.87~8.00 (3H, m), 7.77~7.79 (6H, |
| | m), 7.63~7.69 (6H, m), 7.25~7.52 (14H, m) |
| 2-82 | δ = No ¹H NMR peaks due to 100% deuterium content |
| 2-89 | δ = No ¹H NMR peaks due to 100% deuterium content |
| 2-92 | δ = No ¹H NMR peaks due to 100% deuterium content |
| 2-94 | δ = No ¹H NMR peaks due to 100% deuterium content |
| 2-95 | δ = No ¹H NMR peaks due to 100% deuterium content |
| 3-4 | δ = 8.55 (2H, d), 7.91~7.94 (10H, m), 7.75 (4H, d), 7.35~7.49 (10H, m), |
| | 7.16 (2H, t) |
| 3-12 | δ = 8.55 (2H, d), 7.91~7.94 (5H, m), 7.75 (4H, d), 7.58 (1H, d), |
| | 7.35~7.49 (5H, m), 7.25~7.26 (5H, m), 7.16 (1H, t) |
| 3-26 | δ = 8.55 (1H, d), 8.19 (1H, d), 7.91~7.98 (6H, m), 7.75~7.82 (6H, m), |
| | 7.35~7.57 (13H, m), 7.16~7.25 (3H, m) |
| 3-32 | δ = 8.55 (1H, d), 8.19 (1H, d), 7.91~7.92 (8H, m), 7.75 (4H, d), |
| | 7.35~7.58 (11H, m), 7.16~7.20 (2H, m) |
| 3-53 | δ = 8.55 (2H, d), 8.12 (1H, d), 7.91~7.94 (9H, m), 7.75 (4H, d), |
| | 7.35~7.49 (8H, m), 7.16~7.18 (3H, m) |
| 3-80 | δ = No ¹H NMR peaks due to 100% deuterium content |
| 3-86 | δ = No ¹H NMR peaks due to 100% deuterium content |
| 3-94 | δ = No ¹H NMR peaks due to 100% deuterium content |
| 3-112 | δ = No ¹H NMR peaks due to 100% deuterium content |
| 3-116 | δ = No ¹H NMR peaks due to 100% deuterium content |

TABLE 8

| Compound | FD-Mass | Compound | FD-Mass |
|---|---|---|---|
| 1-1 | m/z = 730.24 (C51H30N4O2 = 730.83) | 1-9 | m/z = 730.24 (C51H30N4O2 = 730.83) |
| 1-12 | m/z = 822.25 (C57H34N2OS = 822.99) | 1-19 | m/z = 818.34 |
| | | | (C57H22D12N4O2 = 819.00) |
| 1-24 | m/z = 757.28 | 1-25 | m/z = 730.24 (C51H30N4O2 = 730.83) |
| | (C51H19D11N2OS = 757.96) | | |
| 1-28 | m/z = 730.24 (C51H30N4O2 = 730.83) | 1-30 | m/z = 806.27 (C57H34N2O2 = 806.93) |
| 1-34 | m/z = 806.27 (C57H34N2O2 = 806.93) | 1-36 | m/z = 806.27 (C57H34N2O2 = 806.93) |
| 1-39 | m/z = 730.24 (C51H30N4O2 = 730.83) | 1-42 | m/z = 730.24 (C51H30N4O2 = 730.83) |
| 1-46 | m/z = 746.21 (C51H30N4OS = 746.89) | 1-50 | m/z = 822.25 (C57H34N4OS = 822.99) |
| 1-52 | m/z = 822.25 (C57H34N4OS = 822.99) | 1-56 | m/z = 822.25 (C57H34N4OS = 822.99) |
| 1-58 | m/z = 746.21 (C51H30N4OS = 746.89) | 1-60 | m/z = 822.25 (C57H34N4OS = 822.99) |
| 1-66 | m/z = 820.36 | 1-67 | m/z = 816.33 |
| | (C57H20D14N4O2 = 821.01) | | (C57H24D10N4O2 = 816.99) |
| 1-69 | m/z = 740.30 | 1-72 | m/z = 741.31 |
| | (C51H20D10N4O2 = 740.89) | | (C51H19D11N4O2 = 741.89) |
| 1-74 | m/z = 835.33 | 1-76 | m/z = 756.28 |
| | (C57H21D13N4OS = 836.07) | | (C51H20D10N4OS = 756.95) |
| 1-78 | m/z = 836.33 | 1-81 | m/z = 730.24 (C51H30N4O2 = 730.83) |
| | (C57H20D14N4OS = 837.07) | | |
| 1-85 | m/z = 806.27 (C57H34N4O2 = 806.27) | 1-87 | m/z = 806.27 (C57H34N4O2 = 806.27) |
| 1-90 | m/z = 806.27 (C57H34N4O2 = 806.27) | 1-92 | m/z = 806.27 (C57H34N4O2 = 806.27) |
| 1-95 | m/z = 730.24 (C51H30N4O2 = 730.83) | 1-98 | m/z = 730.24 (C51H30N4O2 = 730.83) |
| 1-100 | m/z = 806.27 (C57H34N4O2 = 806.93) | 1-103 | m/z = 746.21 (C51H30N4OS = 746.89) |
| 1-106 | m/z = 822.25 (C57H34N4OS = 822.99) | 1-109 | m/z = 822.25 (C57H34N4OS = 822.99) |
| 1-115 | m/z = 746.21 (C51H30N4OS = 746.89) | 1-120 | m/z = 822.25 (C57H34N4OS = 822.99) |
| 1-122 | m/z = 741.31 | 1-127 | m/z = 740.30 |
| | (C51H19D11N4O2 = 741.89) | | (C51H20D10N4O2 = 740.89) |
| 1-131 | m/z = 817.99 | 1-134 | m/z = 758.29 |
| | (C57H23D11N4O2 = 817.99) | | (C51H18D12N4OS = 758.96) |
| 1-136 | m/z = 835.33 | 1-137 | m/z = 730.24 (C51H30N4O2 = 730.83) |
| | (C57H21D13N4OS = 836.07) | | |
| 1-144 | m/z = 806.27 (C57H34N4O2 = 806.93) | 1-148 | m/z = 806.27 (C57H34N4O2 = 806.93) |
| 1-154 | m/z = 730.24 (C51H30N4O2 = 730.83) | 1-158 | m/z = 746.21 (C51H30N4OS = 746.89) |
| 1-161 | m/z = 822.25 (C57H34N4OS = 822.99) | 1-166 | m/z = 822.25 (C57H34N4OS = 822.99) |
| 1-176 | m/z = 822.25 (C57H34N4OS = 822.99) | 1-178 | m/z = 742.31 |
| | | | (C51H18D12N4O2 = 742.90) |
| 1-180 | m/z = 820.36 | 1-186 | m/z = 818.34 |
| | (C57H20D14N4O2 = 821.01) | | (C57H22D12N4O2 = 819.00) |
| 1-189 | m/z = 834.32 | 1-191 | m/z = 759.30 |
| | (C57H22D12N4OS = 835.06) | | (C51H17D13N4OS = 759.97) |
| 1-193 | m/z = 730.24 (C51H30N4O2 = 730.83) | 1-195 | m/z = 730.24 (C51H30N4O2 = 730.83) |
| 1-197 | m/z = 806.27 (C57H34N4O2 = 806.93) | 1-198 | m/z = 806.27 (C57H34N4O2 = 806.93) |
| 1-201 | m/z = 806.27 (C57H34N4O2 = 806.93) | 1-205 | m/z = 730.24 (C51H30N4O2 = 730.83) |
| 1-208 | m/z = 806.27 (C57H34N4O2 = 806.93) | 1-209 | m/z = 806.27 (C57H34N4O2 = 806.93) |
| 1-210 | m/z = 730.24 (C51H30N4O2 = 730.83) | 1-213 | m/z = 746.21 (C51H30N4OS = 746.89) |
| 1-216 | m/z = 746.21 (C51H30N4OS = 746.89) | 1-217 | m/z = 822.25 (C57H34N4OS = 822.99) |
| 1-219 | m/z = 822.25 (C57H34N4OS = 822.99) | 1-222 | m/z = 822.25 (C57H34N4OS = 822.99) |
| 1-224 | m/z = 822.25 (C57H34N4OS = 822.99) | 1-226 | m/z = 746.21 (C51H30N4OS = 746.89) |
| 1-227 | m/z = 746.21 (C51H30N4OS = 746.89) | 1-232 | m/z = 822.25 (C57H34N4OS = 822.99) |
| 1-233 | m/z = 740.30 | 1-236 | m/z = 741.31 |
| | (C51H20D10N4O2 = 740.89) | | (C51H19D11N4O2 = 741.89) |
| 1-238 | m/z = 819.35 | 1-241 | m/z = 741.31 |
| | (C57H21D13N4O2 = 820.00) | | (C51H19D11N4O2 = 741.89) |
| 1-245 | m/z = 757.28 | 1-247 | m/z = 757.28 |
| | (C51H19D11N4OS = 757.96) | | (C51H19D11N4OS = 757.96) |
| 1-248 | m/z = 757.28 | 1-249 | m/z = 730.24 (C51H30N4O2 = 730.83) |
| | (C51H19D11N4OS = 757.96) | | |
| 1-251 | m/z = 730.24 (C51H30N4O2 = 730.83) | 1-253 | m/z = 806.27 (C57H34N4O2 = 806.27) |
| 1-256 | m/z = 806.27 (C57H34N4O2 = 806.27) | 1-257 | m/z = 806.27 (C57H34N4O2 = 806.27) |
| 1-260 | m/z = 806.27 (C57H34N4O2 = 806.27) | 1-263 | m/z = 730.24 (C51H30N4O2 = 730.83) |
| 1-266 | m/z = 730.24 (C51H30N4O2 = 730.83) | 1-270 | m/z = 746.21 (C51H30N4OS = 746.89) |
| 1-273 | m/z = 822.25 (C57H34N4OS = 822.99) | 1-274 | m/z = 822.25 (C57H34N4OS = 822.99) |
| 1-276 | m/z = 822.25 (C57H34N4OS = 822.99) | 1-279 | m/z = 822.25 (C57H34N4OS = 822.99) |
| 1-280 | m/z = 822.25 (C57H34N4OS = 822.99) | 1-287 | m/z = 822.25 (C57H34N4OS = 822.99) |
| 1-289 | m/z = 742.31 | 1-296 | m/z = 741.31 |
| | (C51H18D12N4O2 = 742.90) | | (C51H19D11N4O2 = 741.89) |
| 1-297 | m/z = 837.34 | 1-300 | m/z = 759.30 |
| | (C57H19D15N4OS = 838.08) | | (C51H17D13N4OS = 759.97) |
| 1-301 | m/z = 730.24 (C51H30N4O2 = 730.83) | 1-302 | m/z = 730.24 (C51H30N4O2 = 730.83) |
| 1-305 | m/z = 806.27 (C57H34N4O2 = 806.93) | 1-310 | m/z = 806.27 (C57H34N4O2 = 806.93) |
| 1-312 | m/z = 806.27 (C57H34N4O2 = 806.93) | 1-313 | m/z = 730.24 (C51H30N4O2 = 730.83) |
| 1-316 | m/z = 806.27 (C57H34N4O2 = 806.93) | 1-320 | m/z = 806.27 (C57H34N4O2 = 806.93) |
| 1-321 | m/z = 746.21 (C51H30N4OS = 746.21) | 1-324 | m/z = 746.21 (C51H30N4OS = 746.21) |
| 1-326 | m/z = 822.25 (C57H34N4OS = 822.99) | 1-330 | m/z = 822.25 (C57H34N4OS = 822.99) |
| 1-333 | m/z = 746.21 (C51H30N4OS = 746.21) | 1-337 | m/z = 822.25 (C57H34N4OS = 822.99) |
| 1-341 | m/z = 741.31 | 1-347 | m/z = 818.34 |
| | (C51H19D11N4O2 = 741.89) | | (C57H22D12N4O2 = 819.00) |
| 1-351 | m/z = 833.05 | 1-352 | m/z = 834.32 |
| | (C57H24D10N4OS = 833.05) | | (C57H22D12N4OS = 835.06) |
| 1-355 | m/z = 730.24 (C51H30N4O2 = 730.83) | 1-356 | m/z = 730.24 (C51H30N4O2 = 730.83) |
| 1-360 | m/z = 806.27 (C57H34B4O2 = 806.93) | 1-364 | m/z = 806.27 (C57H34B4O2 = 806.93) |
| 1-368 | m/z = 806.27 (C57H34B4O2 = 806.93) | 1-372 | m/z = 806.27 (C57H34B4O2 = 806.93) |
| 1-373 | m/z = 7846.21 (C51H30N4OS = 746.89) | 1-379 | m/z = 822.25 (C57H34N4OS = 822.99) |
| 1-381 | m/z = 822.25 (C57H34N4OS = 822.99) | 1-388 | m/z = 822.25 (C57H34N4OS = 822.99) |
| 1-395 | m/z = 743.32 | 1-400 | m/z = 741.31 |
| | (C51H17D13N4O2 = 743.91) | | (C51H19D11N4O2 = 741.89) |
| 1-402 | m/z = 757.28 | 1-408 | m/z = 758.29 |
| | (C51H19D11N4OS = 757.96) | | (C51H18D12N4OS = 758.96) |
| 2-1 | m/z = 484.59 (C36H24N2 = 484.19) | 2-16 | m/z = 634.77 (C48H30N2 = 634.24) |
| 2-27 | m/z = 712.88 (C54H36N2 = 712.29) | 2-28 | m/z = 712.88 (C54H36N2 = 712.29) |
| 2-32 | m/z = 636.78 (C48H32N2 = 636.26) | 2-82 | m/z = 668.98 (C48D32N2 = 668.46) |
| 2-89 | m/z = 668.98 (C48D32N2 = 668.46) | 2-92 | m/z = 749.10 (C54D36N2 = 748.51) |
| 2-94 | m/z = 749.10 (C54D36N2 = 748.51) | 2-95 | m/z = 749.10 (C54D36N2 = 748.51) |
| 3-4 | m/z = 560.23 (C42H28N2 = 560.70) | 3-12 | m/z = 636.26 (C48H32N2 = 636.80) |
| 3-26 | m/z = 650.78 (C48H30N2O = 650.24) | 3-32 | m/z = 560.70 (C42H18N2 = 560.23) |
| 3-53 | m/z = 560.23 (C42H28N2 = 560.70) | 3-80 | m/z = 668.46 (C48D32N2 = 668.99) |
| 3-86 | m/z = 588.40 (C42D28N2 = 588.87) | 3-94 | m/z = 588.40 (C42D28N2 = 588.87) |
| 3-112 | m/z = 587.39 (C42HD27N2 = 587.86) | 3-116 | m/z = 588.40 (C42D28N2 = 588.87) |

### Experimental Example 1

### 1) Manufacture of Organic Light Emitting Device

A glass substrate on which ITO was coated as a thin film to a thickness of 1,500 Å was ultrasonic cleaned with distilled water. When the cleaning with distilled water was finished, the substrate was ultrasonic cleaned with solvents such as acetone, methanol and isopropyl alcohol, then dried, and then subjected to UVO treatment for 5 minutes using UV in a UV cleaner. After that, the substrate was transferred to a plasma cleaner (PT), then subjected to plasma treatment under vacuum for ITO work function and residual film removal, and transferred to a thermal deposition apparatus for organic deposition.

On the transparent ITO electrode (positive electrode), 2-TNATA (4,4′,4″-tris[2-naphthyl(phenyl)amino]triphenylamine) that is a hole injection layer and NPB (N,N′-di(1-naphthyl)-N,N′-diphenyl-(1,1′-biphenyl)-4,4′-diamine) that is a hole transport layer, which are common layers, were formed.

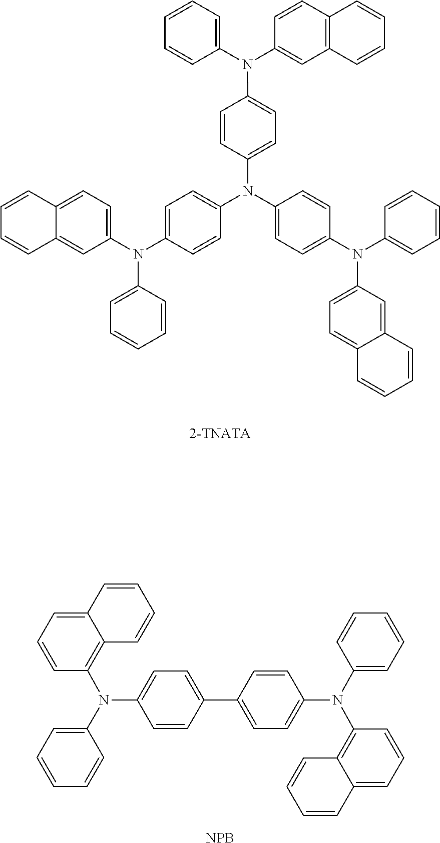 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(N(c2ccc(-c3ccc(N(c4ccccc4)c4cccc5ccccc45)cc3)cc2)c2cccc3ccccc23)cc1 ; "NPB"]

A light emitting layer was thermal vacuum deposited thereon as follows. As the light emitting layer, the heterocyclic compound of Chemical Formula 1 was used as a host and Ir(ppy)₃ (tris(2-phenylpyridine)iridium) was used as a green phosphorescent dopant, and the host was doped with Ir(ppy)₃ by 7% and deposited to 360 Å. After that, BCP was deposited to 60 Å as a hole blocking layer, and Alq₃ was deposited to 200 Å thereon as an electron transport layer. Lastly, lithium fluoride (LiF) was deposited on the electron transport layer to a thickness of 10 Å to form an electron injection layer, and then an aluminum (A1) negative electrode was deposited on the electron injection layer to a thickness of 1,200 Å to form a negative electrode, and as a result, an organic electroluminescent device was manufactured.

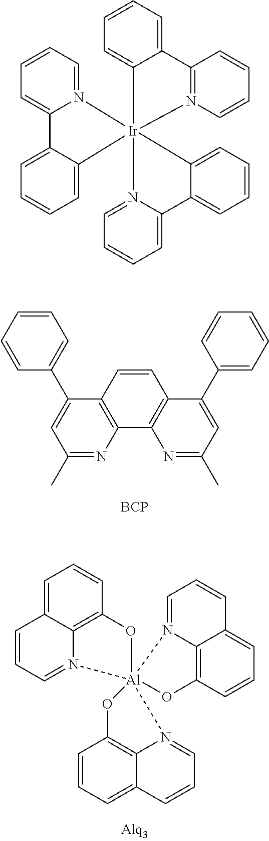

As a red phosphorescent dopant, Ir(piq)₂(acac) was used.

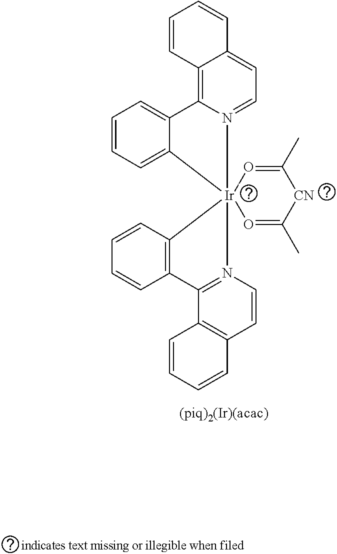

Meanwhile, all the organic compounds required to manufacture the OLED were vacuum sublimation purified under 10⁻⁸ torr to 10⁻⁶ torr for each material to be used in the manufacture of the OLED.

### 2) Driving Voltage and Light Emission Efficiency of Organic Electroluminescent Device

For each of the organic electroluminescent devices manufactured as above, electroluminescent (EL) properties were measured using M7000 manufactured by McScience Inc., and with the measurement results, T₉₀ was measured when standard luminance was 6,000 cd/m² through a lifetime measurement system (M6000) manufactured by McScience Inc. Results of measuring driving voltage, light emission efficiency, color coordinate (CIE) and lifetime of the organic light emitting devices manufactured according to the present disclosure are as shown in Table 9. T₉₀ means a lifetime (unit: h, hour), a time taken for luminance to become 90 with respect to initial luminance.

TABLE 9

| | | Driving | | | |
|---|---|---|---|---|---|
| | | Voltage | Efficiency | Color | Lifetime |
| | Compound | (V) | (cd/A) | Coordinate | (T₉₀) |
| Comparative Example 1 | A | 5.87 | 46.5 | Green | 63 |
| Comparative Example 2 | B | 5.94 | 47.7 | Green | 52 |
| Comparative Example 3 | C | 5.88 | 48.9 | Green | 54 |
| Comparative Example 4 | D | 5.83 | 47.6 | Green | 59 |
| Comparative Example 5 | E | 5.92 | 48.2 | Green | 55 |
| Comparative Example 6 | F | 5.80 | 50.0 | Green | 52 |
| Comparative Example 7 | G | 6.12 | 40.2 | Green | 65 |
| Comparative Example 8 | H | 6.78 | 42.3 | Green | 70 |
| Comparative Example 9 | I | 6.80 | 43.6 | Green | 68 |
| Comparative Example 10 | J | 6.64 | 44.1 | Green | 61 |
| Comparative Example 11 | K | 6.59 | 41.5 | Green | 65 |
| Comparative Example 12 | L | 6.62 | 40.6 | Green | 66 |
| Example 1 | 1-1 | 5.42 | 56.3 | Green | 86 |
| Example 2 | 1-9 | 5.26 | 55.7 | Green | 85 |
| Example 3 | 1-12 | 5.06 | 54.9 | Green | 100 |
| Example 4 | 1-19 | 5.32 | 55.2 | Green | 96 |
| Example 5 | 1-24 | 5.12 | 54.0 | Green | 94 |
| Example 6 | 1-25 | 4.76 | 64.2 | Green | 87 |
| Example 7 | 1-28 | 4.52 | 63.6 | Green | 89 |
| Example 8 | 1-30 | 4.43 | 62.8 | Green | 94 |
| Example 9 | 1-34 | 4.12 | 64.7 | Green | 99 |
| Example 10 | 1-36 | 4.32 | 65.9 | Green | 94 |
| Example 11 | 1-39 | 4.23 | 66.0 | Green | 85 |
| Example 12 | 1-42 | 4.66 | 65.2 | Green | 91 |
| Example 13 | 1-46 | 4.28 | 64.8 | Green | 95 |
| Example 14 | 1-50 | 4.85 | 63.5 | Green | 10 |
| Example 15 | 1-52 | 4.71 | 60.1 | Green | 80 |
| Example 16 | 1-56 | 4.23 | 61.8 | Green | 87 |
| Example 17 | 1-58 | 4.36 | 64.2 | Green | 96 |
| Example 18 | 1-60 | 4.59 | 65.7 | Green | 94 |
| Example 19 | 1-66 | 4.62 | 63.9 | Green | 85 |
| Example 20 | 1-67 | 4.38 | 60.3 | Green | 81 |
| Example 21 | 1-69 | 4.26 | 64.2 | Green | 100 |
| Example 22 | 1-72 | 4.26 | 65.7 | Green | 99 |
| Example 23 | 1-74 | 4.31 | 63.8 | Green | 94 |
| Example 24 | 1-76 | 4.87 | 62.5 | Green | 90 |
| Example 25 | 1-78 | 4.10 | 60.3 | Green | 85 |
| Example 26 | 1-81 | 2.49 | 97.1 | Green | 199 |
| Example 27 | 1-85 | 2.41 | 98.5 | Green | 194 |
| Example 28 | 1-87 | 2.53 | 98.7 | Green | 180 |
| Example 29 | 1-90 | 2.42 | 100.1 | Green | 185 |
| Example 30 | 1-92 | 2.39 | 99.4 | Green | 194 |
| Example 31 | 1-95 | 2.44 | 96.7 | Green | 187 |
| Example 32 | 1-98 | 2.57 | 96.3 | Green | 186 |
| Example 33 | 1-100 | 2.60 | 97.7 | Green | 194 |
| Example 34 | 1-103 | 2.55 | 95.2 | Green | 190 |
| Example 35 | 1-106 | 2.47 | 96.3 | Green | 195 |
| Example 36 | 1-109 | 2.38 | 97.5 | Green | 200 |
| Example 37 | 1-115 | 2.39 | 98.1 | Green | 211 |
| Example 38 | 1-120 | 2.29 | 98.4 | Green | 206 |
| Example 39 | 1-122 | 2.53 | 99.2 | Green | 200 |
| Example 40 | 1-127 | 2.21 | 95.0 | Green | 199 |
| Example 41 | 1-131 | 2.42 | 98.4 | Green | 192 |
| Example 42 | 1-134 | 2.38 | 96.7 | Green | 184 |
| Example 43 | 1-136 | 2.26 | 95.2 | Green | 180 |
| Example 44 | 1-137 | 3.82 | 73.8 | Green | 126 |
| Example 45 | 1-144 | 3.74 | 74.5 | Green | 138 |
| Example 46 | 1-148 | 3.89 | 71.6 | Green | 131 |
| Example 47 | 1-154 | 3.88 | 70.8 | Green | 129 |
| Example 48 | 1-158 | 3.90 | 72.6 | Green | 138 |
| Example 49 | 1-161 | 3.84 | 73.8 | Green | 140 |
| Example 50 | 1-166 | 3.82 | 71.6 | Green | 133 |
| Example 51 | 1-176 | 3.77 | 74.0 | Green | 142 |
| Example 52 | 1-178 | 3.80 | 73.8 | Green | 136 |
| Example 53 | 1-180 | 3.79 | 74.2 | Green | 128 |
| Example 54 | 1-186 | 3.76 | 70.9 | Green | 140 |
| Example 55 | 1-189 | 3.82 | 72.1 | Green | 138 |
| Example 56 | 1-191 | 3.87 | 74.3 | Green | 125 |
| Example 57 | 1-193 | 2.60 | 114.7 | Green | 231 |
| Example 58 | 1-195 | 2.59 | 116.5 | Green | 228 |
| Example 59 | 1-197 | 2.64 | 111.8 | Green | 231 |
| Example 60 | 1-198 | 2.84 | 110.7 | Green | 239 |
| Example 61 | 1-201 | 2.82 | 112.6 | Green | 236 |
| Example 62 | 1-205 | 2.94 | 106.8 | Green | 235 |
| Example 63 | 1-208 | 2.91 | 119.7 | Green | 225 |
| Example 64 | 1-209 | 2.93 | 120.0 | Green | 237 |
| Example 65 | 1-210 | 2.88 | 116.5 | Green | 240 |
| Example 66 | 1-213 | 2.82 | 113.2 | Green | 239 |
| Example 67 | 1-216 | 2.74 | 109.7 | Green | 237 |
| Example 68 | 1-217 | 2.68 | 115.4 | Green | 225 |
| Example 69 | 1-219 | 2.74 | 119.7 | Green | 240 |
| Example 70 | 1-222 | 2.63 | 112.8 | Green | 233 |
| Example 71 | 1-224 | 2.71 | 111.6 | Green | 231 |
| Example 72 | 1-226 | 2.83 | 109.4 | Green | 225 |
| Example 73 | 1-227 | 2.77 | 106.8 | Green | 238 |
| Example 74 | 1-232 | 2.86 | 117.3 | Green | 230 |
| Example 75 | 1-233 | 3.01 | 118.4 | Green | 229 |
| Example 76 | 1-236 | 2.69 | 121.6 | Green | 226 |
| Example 77 | 1-238 | 2.83 | 105.3 | Green | 240 |
| Example 78 | 1-241 | 2.89 | 108.7 | Green | 241 |
| Example 79 | 1-245 | 2.76 | 120.3 | Green | 238 |
| Example 80 | 1-247 | 2.60 | 112.8 | Green | 226 |
| Example 81 | 1-248 | 2.68 | 105.6 | Green | 220 |
| Example 82 | 1-249 | 3.37 | 93.7 | Green | 172 |
| Example 83 | 1-251 | 3.26 | 92.6 | Green | 175 |
| Example 84 | 1-253 | 3.13 | 91.4 | Green | 169 |
| Example 85 | 1-256 | 3.09 | 93.5 | Green | 154 |
| Example 86 | 1-257 | 3.08 | 92.4 | Green | 165 |
| Example 87 | 1-260 | 3.18 | 91.6 | Green | 168 |
| Example 88 | 1-263 | 3.54 | 87.4 | Green | 180 |
| Example 89 | 1-266 | 3.66 | 88.6 | Green | 177 |
| Example 90 | 1-270 | 3.70 | 90.5 | Green | 172 |
| Example 91 | 1-273 | 3.68 | 91.6 | Green | 175 |
| Example 92 | 1-274 | 3.59 | 92.7 | Green | 164 |
| Example 93 | 1-276 | 3.64 | 93.4 | Green | 161 |
| Example 94 | 1-279 | 3.52 | 94.1 | Green | 154 |
| Example 95 | 1-280 | 3.33 | 85.2 | Green | 166 |
| Example 96 | 1-287 | 3.42 | 83.4 | Green | 165 |
| Example 97 | 1-289 | 3.37 | 82.6 | Green | 180 |
| Example 98 | 1-296 | 3.25 | 88.7 | Green | 174 |
| Example 99 | 1-297 | 3.12 | 90.4 | Green | 162 |
| Example 100 | 1-300 | 3.02 | 94.2 | Green | 150 |
| Example 101 | 1-301 | 3.32 | 78.3 | Green | 162 |
| Example 102 | 1-302 | 3.11 | 79.6 | Green | 149 |
| Example 103 | 1-305 | 3.28 | 80.5 | Green | 151 |
| Example 104 | 1-310 | 3.41 | 78.2 | Green | 152 |
| Example 105 | 1-312 | 3.68 | 79.4 | Green | 168 |
| Example 106 | 1-313 | 3.52 | 75.6 | Green | 162 |
| Example 107 | 1-316 | 3.66 | 77.6 | Green | 144 |
| Example 108 | 1-320 | 3.28 | 80.1 | Green | 146 |
| Example 109 | 1-321 | 3.15 | 78.2 | Green | 159 |
| Example 110 | 1-324 | 3.05 | 79.5 | Green | 158 |
| Example 111 | 1-326 | 3.68 | 81.5 | Green | 154 |
| Example 112 | 1-330 | 3.42 | 80.6 | Green | 152 |
| Example 113 | 1-333 | 3.25 | 76.2 | Green | 170 |
| Example 114 | 1-337 | 3.70 | 77.3 | Green | 169 |
| Example 115 | 1-341 | 3.69 | 79.4 | Green | 165 |
| Example 116 | 1-347 | 3.29 | 76.5 | Green | 164 |
| Example 117 | 1-351 | 3.15 | 80.4 | Green | 145 |
| Example 118 | 1-352 | 3.23 | 75.1 | Green | 140 |
| Example 119 | 1-355 | 4.55 | 68.4 | Green | 120 |
| Example 120 | 1-356 | 4.61 | 69.2 | Green | 121 |
| Example 121 | 1-360 | 4.76 | 69.5 | Green | 130 |
| Example 122 | 1-364 | 4.81 | 70.4 | Green | 129 |
| Example 123 | 1-368 | 4.94 | 66.0 | Green | 105 |
| Example 124 | 1-372 | 4.83 | 67.5 | Green | 113 |
| Example 125 | 1-373 | 4.72 | 68.4 | Green | 121 |
| Example 126 | 1-379 | 4.68 | 69.2 | Green | 119 |
| Example 127 | 1-381 | 4.45 | 66.5 | Green | 105 |
| Example 128 | 1-388 | 4.43 | 68.4 | Green | 100 |
| Example 129 | 1-395 | 4.09 | 69.1 | Green | 128 |
| Example 130 | 1-400 | 4.26 | 70.3 | Green | 125 |
| Example 131 | 1-402 | 4.13 | 68.4 | Green | 121 |
| Example 132 | 1-408 | 4.05 | 66.2 | Green | 117 |

The comparative compounds used in Table 9 are as follows.

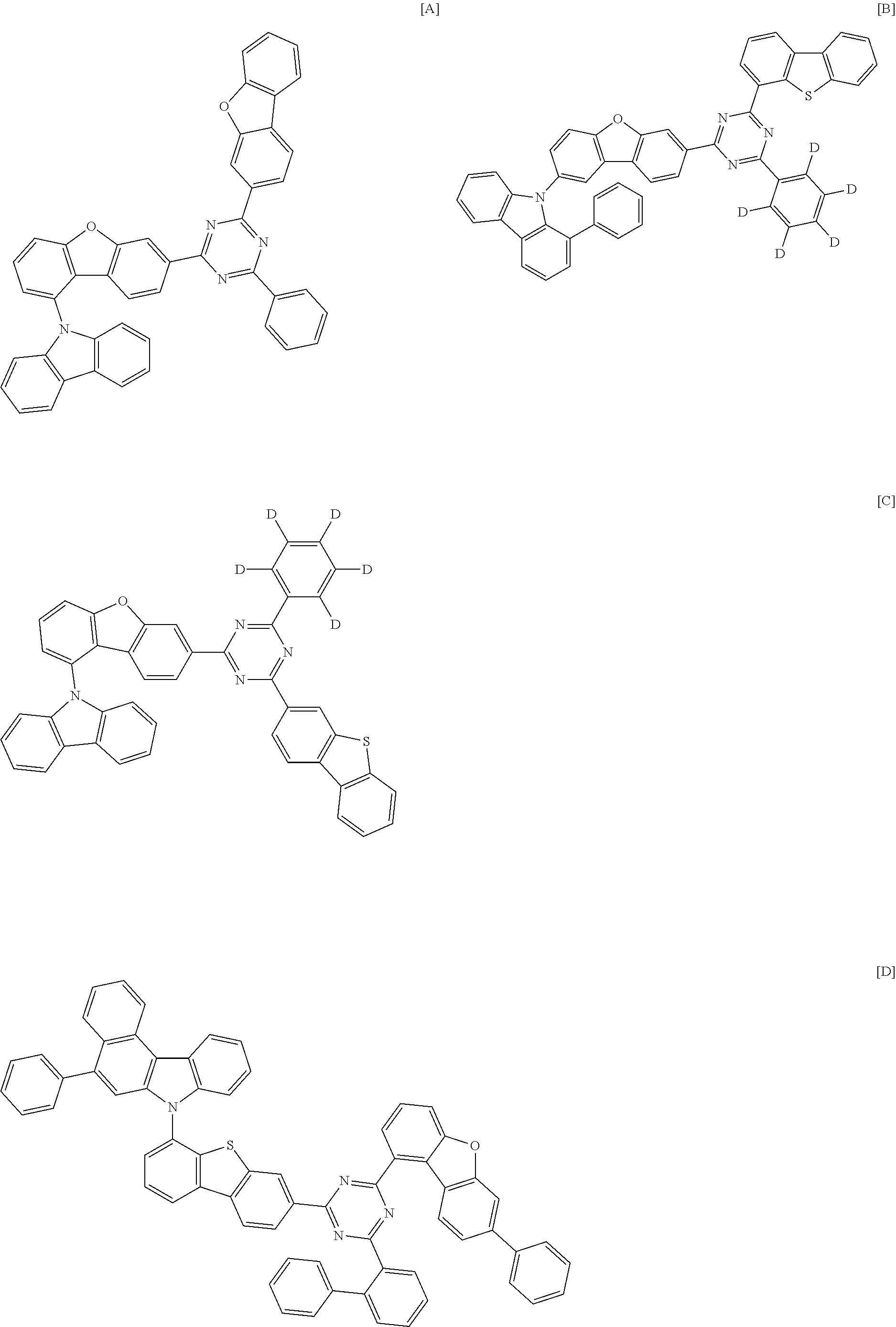 [SMILES: c1ccc(-c2nc(-c3ccc4c(c3)oc3ccccc34)nc(-c3ccc4c(c3)oc3cccc(-n5c6ccccc6c6ccccc65)c34)n2)cc1 ; [2H]c1c([2H])c([2H])c(-c2nc(-c3ccc4c(c3)oc3ccc(-n5c6ccccc6c6cccc(-c7ccccc7)c65)cc34)nc(-c3cccc4c3sc3ccccc34)n2)c([2H])c1[2H] ; [2H]c1c([2H])c([2H])c(-c2nc(-c3ccc4c(c3)oc3cccc(-n5c6ccccc6c6ccccc65)c34)nc(-c3ccc4c(c3)sc3ccccc34)n2)c([2H])c1[2H] ; c1ccc(-c2ccc3c(c2)oc2cccc(-c4nc(-c5ccc6c(c5)sc5c(-n7c8ccccc8c8c9ccccc9c(-c9ccccc9)cc87)cccc56)nc(-c5ccccc5-c5ccccc5)n4)c23)cc1]

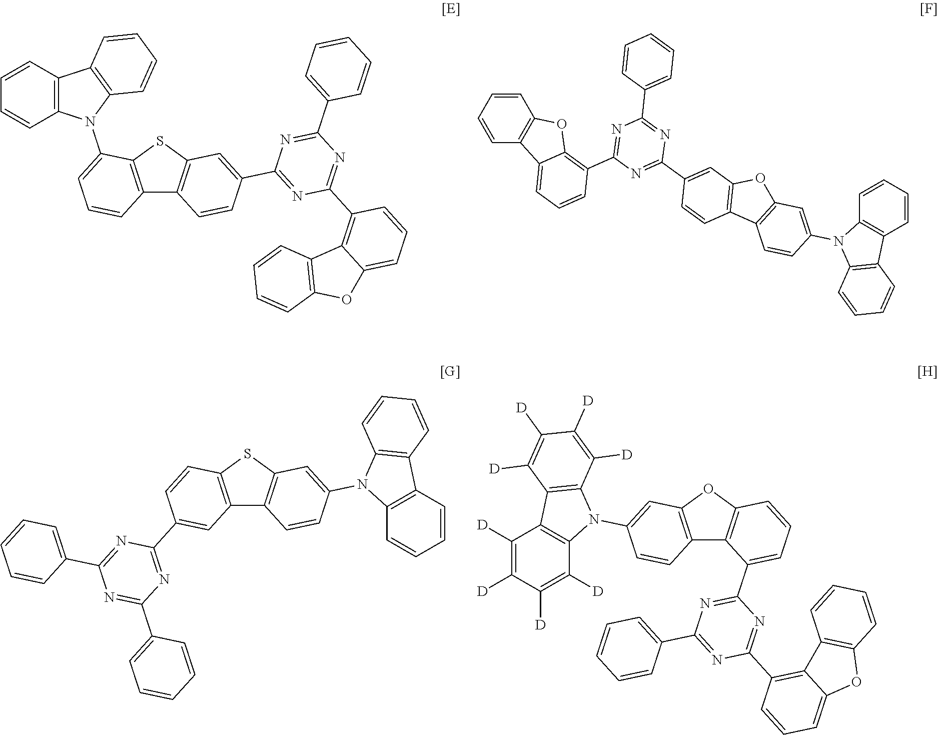

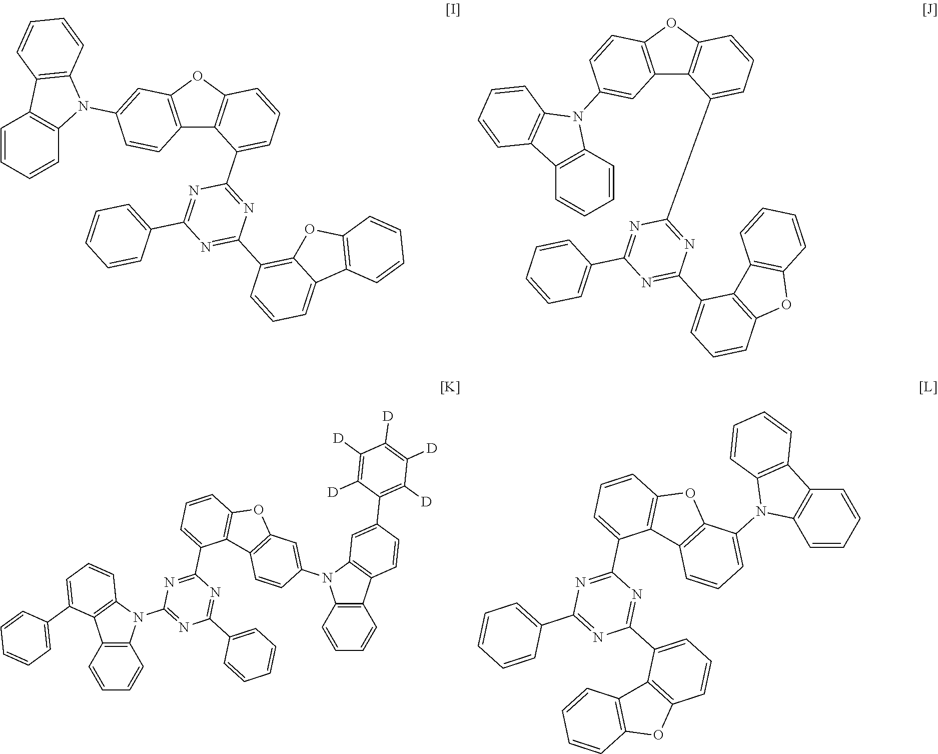 [SMILES: c1ccc(-c2nc(-c3cccc4c3oc3ccccc34)nc(-c3cccc4oc5cc(-n6c7ccccc7c7ccccc76)ccc5c34)n2)cc1 ; c1ccc(-c2nc(-c3cccc4oc5ccccc5c34)nc(-c3cccc4oc5ccc(-n6c7ccccc7c7ccccc76)cc5c34)n2)cc1 ; [2H]c1c([2H])c([2H])c(-c2ccc3c4ccccc4n(-c4ccc5c(c4)oc4cccc(-c6nc(-c7ccccc7)nc(-n7c8ccccc8c8c(-c9ccccc9)cccc87)n6)c45)c3c2)c([2H])c1[2H] ; c1ccc(-c2nc(-c3cccc4oc5ccccc5c34)nc(-c3cccc4oc5c(-n6c7ccccc7c7ccccc76)cccc5c34)n2)cc1]

Examining the results of Table 9, it can be seen that the organic light emitting devices including the heterocyclic compound of Chemical Formula 1 of the present disclosure are superior in all aspects of driving voltage, light emission efficiency and lifetime compared to the organic light emitting devices of Comparative Examples.

### Experimental Example 2

### 1) Manufacture of Organic Light Emitting Device

A glass substrate on which ITO was coated as a thin film to a thickness of 1,500 Å was ultrasonic cleaned with distilled water. After the cleaning with distilled water was finished, the substrate was ultrasonic cleaned with solvents such as acetone, methanol and isopropyl alcohol, then dried, and then subjected to UVO treatment for 5 minutes using UV in a UV cleaner. After that, the substrate was transferred to a plasma cleaner (PT), then subjected to plasma treatment under vacuum for ITO work function and residual film removal, and transferred to a thermal deposition apparatus for organic deposition.

On the transparent ITO electrode (positive electrode), 2-TNATA (4,4′,4″-tris[2-naphthyl(phenyl)amino]triphenylamine) that is a hole injection layer and NPB (N,N′-di(1-naphthyl)-N,N′-diphenyl-(1,1′-biphenyl)-4,4′-diamine) that is a hole transport layer, which are common layers, were formed.

A light emitting layer was thermal vacuum deposited thereon as follows. As the light emitting layer, one type of the heterocyclic compound of Chemical Formula 1 and the heterocyclic compound represented by Chemical Formula 2 or 3 were pre-mixed and deposited to 360 Å in one source of supply as a host, and as a green phosphorescent dopant, Ir(ppy)₃ was doped and deposited by 7% with respect to the deposition thickness of the light emitting layer. After that, BCP was deposited to 60 Å as a hole blocking layer, and Alq₃ was deposited to 200 Å thereon as an electron transport layer. Lastly, lithium fluoride (LiF) was deposited on the electron transport layer to a thickness of 10 Å to form an electron injection layer, and then an aluminum (A1) negative electrode was deposited on the electron injection layer to a thickness of 1,200 Å to form a negative electrode, and as a result, an organic electroluminescent device was manufactured.

In the following Table 10, green hosts were used in Examples and Comparative Examples other than those separately indicated as red hosts. As the red phosphorescent dopant, Ir(piq)₂(acac) was used.

Meanwhile, all the organic compounds required to manufacture the OLED were vacuum sublimation purified under 10⁻⁸ torr to 10⁻⁶ torr for each material to be used in the manufacture of the OLED.

### 2) Driving Voltage and Light Emission Efficiency of Organic Electroluminescent Device

For each of the organic electroluminescent devices manufactured as above, electroluminescent (EL) properties were measured using M7000 manufactured by McScience Inc., and with the measurement results, T₉₀ was measured when standard luminance was 6,000 cd/m² through a lifetime measurement system (M6000) manufactured by McScience Inc.

Results of measuring driving voltage, light emission efficiency, color coordinate (CIE) and lifetime of the organic light emitting devices manufactured according to the present disclosure are as shown in Table 10. T₉₀ means a lifetime (unit: h, hour), a time taken for luminance to become 90% with respect to initial luminance.

TABLE 10

| | | | Driving | Efficiency | Color | Lifetime |
|---|---|---|---|---|---|---|
| | Compound | Ratio | Voltage (V) | (cd/A) | Coordinate | (T₉₀) |
| Comparative | [A]:2-16 | 1:1 | 4.44 | 62.5 | Green | 80 |
| Example 13 | | | | | | |
| Comparative | | 1:2 | 4.52 | 61.8 | Green | 83 |
| Example 14 | | | | | | |
| Comparative | | 1:3 | 4.56 | 60.5 | Green | 86 |
| Example 15 | | | | | | |
| Comparative | [B]:2-27 | 1:1 | 4.38 | 63.3 | Green | 82 |
| Example 16 | | | | | | |
| Comparative | | 1:2 | 4.41 | 63.1 | Green | 86 |
| Example 17 | | | | | | |
| Comparative | | 1:3 | 4.45 | 62.5 | Green | 89 |
| Example 18 | | | | | | |
| Comparative | [B]:2-89 | 1:1 | 4.53 | 64.8 | Green | 81 |
| Example 19 | | | | | | |
| Comparative | | 1:2 | 4.56 | 63.2 | Green | 83 |
| Example 20 | | | | | | |
| Comparative | | 1:3 | 4.58 | 62.1 | Green | 89 |
| Example 21 | | | | | | |
| Comparative | [C]:3-12 | 1:1 | 4.42 | 60.5 | Green | 82 |
| Example 22 | | | | | | |
| Comparative | | 1:2 | 4.49 | 59.7 | Green | 84 |
| Example 23 | | | | | | |
| Comparative | | 1:3 | 4.53 | 58.2 | Green | 86 |
| Example 24 | | | | | | |
| Comparative | [D]:2-95 | 1:1 | 4.32 | 64.2 | Green | 81 |
| Example 25 | | | | | | |
| Comparative | | 1:2 | 4.38 | 63.5 | Green | 84 |
| Example 26 | | | | | | |
| Comparative | | 1:3 | 4.46 | 62.3 | Green | 86 |
| Example 27 | | | | | | |
| Comparative | [E]:3-94 | 1:1 | 4.39 | 64.8 | Green | 83 |
| Example 28 | | | | | | |
| Comparative | | 1:2 | 4.41 | 63.7 | Green | 85 |
| Example 29 | | | | | | |
| Comparative | | 1:3 | 4.45 | 62.5 | Green | 87 |
| Example 30 | | | | | | |
| Comparative | [F]:3-116 | 1:1 | 4.32 | 65.0 | Green | 81 |
| Example 31 | | | | | | |
| Comparative | | 1:2 | 4.35 | 64.2 | Green | 83 |
| Example 32 | | | | | | |
| Comparative | | 1:3 | 4.42 | 63.8 | Green | 85 |
| Example 33 | | | | | | |
| Comparative | [G]:3-32 | 1:1 | 4.87 | 60.5 | Green | 84 |
| Example 34 | | | | | | |
| Comparative | | 1:2 | 4.91 | 59.3 | Green | 86 |
| Example 35 | | | | | | |
| Comparative | | 1:3 | 4.93 | 58.1 | Green | 88 |
| Example 36 | | | | | | |
| Comparative | [H]:3-53 | 1:1 | 5.12 | 58.8 | Green | 90 |
| Example 37 | | | | | | |
| Comparative | | 1:2 | 5.18 | 57.4 | Green | 92 |
| Example 38 | | | | | | |
| Comparative | | 1:3 | 5.21 | 56.8 | Green | 95 |
| Example 39 | | | | | | |
| Comparative | [I]:3-12 | 1:1 | 5.22 | 59.7 | Green | 94 |
| Example 40 | | | | | | |
| Comparative | | 1:2 | 5.26 | 58.3 | Green | 96 |
| Example 41 | | | | | | |
| Comparative | | 1:3 | 5.29 | 57.1 | Green | 99 |
| Example 42 | | | | | | |
| Comparative | [J]:2-89 | 1:1 | 5.03 | 58.2 | Green | 89 |
| Example 43 | | | | | | |
| Comparative | | 1:2 | 5.12 | 57.4 | Green | 90 |
| Example 44 | | | | | | |
| Comparative | | 1:3 | 5.19 | 56.1 | Green | 94 |
| Example 45 | | | | | | |
| Comparative | [K]:2-95 | 1:1 | 5.11 | 59.4 | Green | 94 |
| Example 46 | | | | | | |
| Comparative | | 1:2 | 5.18 | 58.2 | Green | 96 |
| Example 47 | | | | | | |
| Comparative | | 1:3 | 5.21 | 57.4 | Green | 99 |
| Example 48 | | | | | | |
| Comparative | [L]:3-112 | 1:1 | 5.06 | 60.8 | Green | 87 |
| Example 49 | | | | | | |
| Comparative | | 1:2 | 5.13 | 59.2 | Green | 89 |
| Example 50 | | | | | | |
| Comparative | | 1:3 | 5.18 | 58.4 | Green | 94 |
| Example 51 | | | | | | |
| Example 133 | 1-1:2-95 | 1:1 | 3.80 | 75.2 | Green | 110 |
| Example 134 | | 1:2 | 3.82 | 74.6 | Green | 115 |
| Example 135 | | 1:3 | 3.83 | 74.1 | Green | 118 |
| Example 136 | 1-9:2-28 | 1:1 | 3.94 | 78.5 | Green | 123 |
| Example 137 | | 1:2 | 3.99 | 78.1 | Green | 126 |
| Example 138 | | 1:3 | 4.01 | 77.6 | Green | 127 |
| Example 139 | 1-12:3-26 | 1:1 | 3.78 | 74.9 | Green | 129 |
| Example 140 | | 1:2 | 3.80 | 74.5 | Green | 132 |
| Example 141 | | 1:3 | 3.81 | 74.3 | Green | 139 |
| Example 142 | 1-19:3-53 | 1:1 | 3.92 | 80.6 | Green | 115 |
| Example 143 | | 1:2 | 3.94 | 79.5 | Green | 119 |
| Example 144 | | 1:3 | 3.98 | 78.7 | Green | 121 |
| Example 145 | 1-24:3-12 | 1:1 | 3.87 | 77.6 | Green | 119 |
| Example 146 | | 1:2 | 3.89 | 76.3 | Green | 125 |
| Example 147 | | 1:3 | 3.94 | 75.2 | Green | 130 |
| Example 148 | 1-25:2-95 | 1:1 | 3.81 | 79.6 | Green | 115 |
| Example 149 | | 1:2 | 3.84 | 78.4 | Green | 116 |
| Example 150 | | 1:3 | 3.89 | 78.1 | Green | 120 |
| Example 151 | 1-28:2-82 | 1:1 | 3.78 | 80.6 | Green | 123 |
| Example 152 | | 1:2 | 3.80 | 79.4 | Green | 125 |
| Example 153 | | 1:3 | 3.82 | 78.5 | Green | 129 |
| Example 154 | 1-30:3-53 | 1:1 | 3.85 | 82.6 | Green | 127 |
| Example 155 | | 1:2 | 3.88 | 81.3 | Green | 129 |
| Example 156 | | 1:3 | 3.94 | 80.9 | Green | 132 |
| Example 157 | 1-34:2-82 | 1:1 | 3.91 | 82.4 | Green | 122 |
| Example 158 | | 1:2 | 3.94 | 83.5 | Green | 126 |
| Example 159 | | 1:3 | 3.96 | 80.3 | Green | 129 |
| Example 160 | 1-36:2-94 | 1:1 | 3.81 | 79.9 | Green | 120 |
| Example 161 | | 1:2 | 3.85 | 78.1 | Green | 126 |
| Example 162 | | 1:3 | 3.87 | 78.0 | Green | 130 |
| Example 163 | 1-39:2-95 | 1:1 | 3.89 | 79.6 | Green | 111 |
| Example 164 | | 1:2 | 3.91 | 78.4 | Green | 119 |
| Example 165 | | 1:3 | 3.96 | 77.3 | Green | 123 |
| Example 166 | 1-39:3-53 | 1:1 | 3.92 | 78.5 | Green | 117 |
| Example 167 | | 1:2 | 3.95 | 77.3 | Green | 120 |
| Example 168 | | 1:3 | 3.99 | 76.1 | Green | 126 |
| Example 169 | 1-42:3-80 | 1:1 | 3.87 | 75.3 | Green | 132 |
| Example 170 | | 1:2 | 3.89 | 74.2 | Green | 135 |
| Example 171 | | 1:3 | 3.93 | 73.9 | Green | 136 |
| Example 172 | 1-46:2-95 | 1:1 | 3.90 | 82.0 | Green | 128 |
| Example 173 | | 1:2 | 3.93 | 81.9 | Green | 131 |
| Example 174 | | 1:3 | 3.95 | 81.2 | Green | 134 |
| Example 175 | 1-50:3-26 | 1:1 | 3.81 | 78.4 | Green | 116 |
| Example 176 | | 1:2 | 3.85 | 77.5 | Green | 119 |
| Example 177 | | 1:3 | 3.89 | 77.1 | Green | 123 |
| Example 178 | 1-52:2-89 | 1:1 | 3.90 | 74.6 | Green | 111 |
| Example 179 | | 1:2 | 3.93 | 73.5 | Green | 115 |
| Example 180 | | 1:3 | 3.99 | 72.8 | Green | 119 |
| Example 181 | 1-56:2-95 | 1:1 | 3.85 | 75.2 | Green | 118 |
| Example 182 | | 1:2 | 3.87 | 74.7 | Green | 122 |
| Example 183 | | 1:3 | 3.92 | 73.5 | Green | 129 |
| Example 184 | 1-58:3-116 | 1:1 | 3.81 | 76.9 | Green | 123 |
| Example 185 | | 1:2 | 3.83 | 76.1 | Green | 128 |
| Example 186 | | 1:3 | 3.86 | 75.4 | Green | 132 |
| Example 187 | 1-60:3-26 | 1:1 | 3.87 | 77.3 | Green | 111 |
| Example 188 | | 1:2 | 3.89 | 76.1 | Green | 119 |
| Example 189 | | 1:3 | 3.94 | 75.5 | Green | 120 |
| Example 190 | 1-66:3-53 | 1:1 | 3.80 | 78.3 | Green | 115 |
| Example 191 | | 1:2 | 3.83 | 77.9 | Green | 119 |
| Example 192 | | 1:3 | 3.85 | 76.0 | Green | 121 |
| Example 193 | 1-67:2-32 | 1:1 | 3.91 | 81.6 | Green | 118 |
| Example 194 | | 1:2 | 3.94 | 80.4 | Green | 123 |
| Example 195 | | 1:3 | 3.97 | 79.8 | Green | 128 |
| Example 196 | 1-69:3-94 | 1:1 | 3.78 | 81.7 | Green | 110 |
| Example 197 | | 1:2 | 3.82 | 81.5 | Green | 115 |
| Example 198 | | 1:3 | 3.86 | 80.6 | Green | 119 |
| Example 199 | 1-72:2-32 | 1:1 | 3.89 | 82.0 | Green | 117 |
| Example 200 | | 1:2 | 3.92 | 81.4 | Green | 120 |
| Example 201 | | 1:3 | 3.94 | 80.3 | Green | 123 |
| Example 202 | 1-74:3-53 | 1:1 | 3.79 | 79.5 | Green | 114 |
| Example 203 | | 1:2 | 3.92 | 78.3 | Green | 116 |
| Example 204 | | 1:3 | 3.98 | 77.1 | Green | 119 |
| Example 205 | 1-76:3-80 | 1:1 | 3.82 | 75.6 | Green | 132 |
| Example 206 | | 1:2 | 3.86 | 74.3 | Green | 139 |
| Example 207 | | 1:3 | 3.89 | 73.1 | Green | 140 |
| Example 208 | 1-78:3-94 | 1:1 | 3.91 | 79.5 | Green | 131 |
| Example 209 | | 1:2 | 3.94 | 78.3 | Green | 133 |
| Example 210 | | 1:3 | 3.96 | 77.6 | Green | 138 |
| Example 211 | 1-81:2-92 | 1:1 | 1.87 | 152.3 | Green | 230 |
| Example 212 | | 1:2 | 1.89 | 151.6 | Green | 233 |
| Example 213 | | 1:3 | 1.93 | 150.3 | Green | 238 |
| Example 214 | 1-85:2-32 | 1:1 | 1.74 | 160.7 | Green | 241 |
| Example 215 | | 1:2 | 1.77 | 159.4 | Green | 244 |
| Example 216 | | 1:3 | 1.80 | 158.2 | Green | 249 |
| Example 217 | 1-87:2-89 | 1:1 | 1.65 | 157.7 | Green | 236 |
| Example 218 | | 1:2 | 1.69 | 156.3 | Green | 237 |
| Example 219 | | 1:3 | 1.73 | 155.2 | Green | 240 |
| Example 220 | 1-90:3-32 | 1:1 | 1.23 | 160.1 | Green | 239 |
| Example 221 | | 1:2 | 1.33 | 158.4 | Green | 241 |
| Example 222 | | 1:3 | 1.39 | 157.3 | Green | 244 |
| Example 223 | 1-92:2-82 | 1:1 | 1.32 | 150.9 | Green | 251 |
| Example 224 | | 1:2 | 1.38 | 149.4 | Green | 255 |
| Example 225 | | 1:3 | 1.43 | 148.3 | Green | 259 |
| Example 226 | 1-95:3-112 | 1:1 | 1.44 | 152.3 | Green | 246 |
| Example 227 | | 1:2 | 1.47 | 151.7 | Green | 249 |
| Example 228 | | 1:3 | 1.50 | 150.3 | Green | 253 |
| Example 229 | 1-98:3-116 | 1:1 | 1.32 | 149.7 | Green | 231 |
| Example 230 | | 1:2 | 1.38 | 146.5 | Green | 233 |
| Example 231 | | 1:3 | 1.41 | 143.2 | Green | 238 |
| Example 232 | 1-100:3-26 | 1:1 | 1.43 | 149.5 | Green | 244 |
| Example 233 | | 1:2 | 1.46 | 148.3 | Green | 246 |
| Example 234 | | 1:3 | 1.49 | 147.1 | Green | 247 |
| Example 235 | 1-103:2-92 | 1:1 | 1.12 | 153.8 | Green | 252 |
| Example 236 | | 1:2 | 1.18 | 152.6 | Green | 255 |
| Example 237 | | 1:3 | 1.21 | 151.3 | Green | 257 |
| Example 238 | 1-106:2-82 | 1:1 | 1.18 | 151.6 | Green | 241 |
| Example 239 | | 1:2 | 1.22 | 150.4 | Green | 246 |
| Example 240 | | 1:3 | 1.25 | 149.3 | Green | 249 |
| Example 241 | 1-109:2-4 | 1:1 | 1.08 | 159.3 | Green | 244 |
| Example 242 | | 1:2 | 1.13 | 158.1 | Green | 246 |
| Example 243 | | 1:3 | 1.19 | 157.7 | Green | 248 |
| Example 244 | 1-115:2-28 | 1:1 | 1.11 | 155.6 | Green | 250 |
| Example 245 | | 1:2 | 1.16 | 154.3 | Green | 253 |
| Example 246 | | 1:3 | 1.18 | 153.8 | Green | 255 |
| Example 247 | 1-120:2-53 | 1:1 | 1.13 | 160.4 | Green | 253 |
| Example 248 | | 1:2 | 1.19 | 159.3 | Green | 259 |
| Example 249 | | 1:3 | 1.21 | 158.1 | Green | 260 |
| Example 250 | 1-122:2-94 | 1:1 | 1.23 | 157.9 | Green | 248 |
| Example 251 | | 1:2 | 1.29 | 155.3 | Green | 251 |
| Example 252 | | 1:3 | 1.30 | 154.6 | Green | 255 |
| Example 253 | 1-127:3-53 | 1:1 | 1.37 | 148.7 | Green | 231 |
| Example 254 | | 1:2 | 1.40 | 147.6 | Green | 237 |
| Example 255 | | 1:3 | 1.42 | 146.2 | Green | 240 |
| Example 256 | 1-131:2-89 | 1:1 | 1.29 | 145.3 | Green | 233 |
| Example 257 | | 1:2 | 1.32 | 144.1 | Green | 239 |
| Example 258 | | 1:3 | 1.35 | 143.8 | Green | 242 |
| Example 259 | 1-134:2-86 | 1:1 | 1.08 | 153.6 | Green | 246 |
| Example 260 | | 1:2 | 1.12 | 152.7 | Green | 249 |
| Example 261 | | 1:3 | 1.15 | 151.6 | Green | 253 |
| Example 262 | 1-136:2-92 | 1:1 | 1.18 | 150.7 | Green | 246 |
| Example 263 | | 1:2 | 1.23 | 149.3 | Green | 249 |
| Example 264 | | 1:3 | 1.32 | 148.1 | Green | 253 |
| Example 265 | 1-137:2-95 | 1:1 | 3.50 | 92.0 | Green | 172 |
| Example 266 | | 1:2 | 3.52 | 91.8 | Green | 175 |
| Example 267 | | 1:3 | 3.55 | 91.1 | Green | 177 |
| Example 268 | 1-144:2-16 | 1:1 | 3.68 | 91.5 | Green | 171 |
| Example 269 | | 1:2 | 3.70 | 91.3 | Green | 173 |
| Example 270 | | 1:3 | 3.71 | 90.0 | Green | 176 |
| Example 271 | 1-144:3-12 | 1:1 | 3.54 | 87.4 | Green | 169 |
| Example 272 | | 1:2 | 3.56 | 87.1 | Green | 171 |
| Example 273 | | 1:3 | 3.59 | 86.5 | Green | 172 |
| Example 274 | 1-148:2-89 | 1:1 | 3.61 | 86.9 | Green | 179 |
| Example 275 | | 1:2 | 3.64 | 86.3 | Green | 180 |
| Example 276 | | 1:3 | 3.66 | 85.7 | Green | 183 |
| Example 277 | 1-154:3-32 | 1:1 | 3.56 | 85.5 | Green | 175 |
| Example 278 | | 1:2 | 3.57 | 85.1 | Green | 179 |
| Example 279 | | 1:3 | 3.59 | 84.9 | Green | 182 |
| Example 280 | 1-158:3-112 | 1:1 | 3.55 | 81.6 | Green | 162 |
| Example 281 | | 1:2 | 3.56 | 81.4 | Green | 166 |
| Example 282 | | 1:3 | 3.61 | 80.5 | Green | 169 |
| Example 283 | 1-161:3-94 | 1:1 | 3.62 | 87.6 | Green | 165 |
| Example 284 | | 1:2 | 3.65 | 86.4 | Green | 167 |
| Example 285 | | 1:3 | 3.66 | 85.2 | Green | 170 |
| Example 286 | 1-166:3-80 | 1:1 | 3.51 | 90.4 | Green | 168 |
| Example 287 | | 1:2 | 3.53 | 89.7 | Green | 171 |
| Example 288 | | 1:3 | 3.56 | 88.5 | Green | 179 |
| Example 289 | 1-176:3-94 | 1:1 | 3.64 | 87.4 | Green | 166 |
| Example 290 | | 1:2 | 3.66 | 86.3 | Green | 169 |
| Example 291 | | 1:3 | 3.68 | 85.5 | Green | 172 |
| Example 292 | 1-178:2-94 | 1:1 | 3.59 | 86.4 | Green | 163 |
| Example 293 | | 1:2 | 3.61 | 85.2 | Green | 168 |
| Example 294 | | 1:3 | 3.64 | 84.9 | Green | 172 |
| Example 295 | 1-180:2-95 | 1:1 | 3.55 | 85.6 | Green | 171 |
| Example 296 | | 1:2 | 3.57 | 84.7 | Green | 176 |
| Example 297 | | 1:3 | 3.59 | 83.5 | Green | 179 |
| Example 298 | 1-186:3-80 | 1:1 | 3.51 | 84.4 | Green | 168 |
| Example 299 | | 1:2 | 3.53 | 83.6 | Green | 173 |
| Example 300 | | 1:3 | 3.56 | 83.1 | Green | 177 |
| Example 301 | 1-189:3-112 | 1:1 | 3.64 | 88.5 | Green | 166 |
| Example 302 | | 1:2 | 3.66 | 87.4 | Green | 169 |
| Example 303 | | 1:3 | 3.68 | 86.5 | Green | 172 |
| Example 304 | 1-191:3-94 | 1:1 | 3.51 | 84.7 | Green | 160 |
| Example 305 | | 1:2 | 3.54 | 83.5 | Green | 162 |
| Example 306 | | 1:3 | 3.59 | 82.1 | Green | 165 |
| Example 307 | 1-193:2-27 | 1:1 | 2.02 | 174.3 | Green | 260 |
| Example 308 | | 1:2 | 2.05 | 173.2 | Green | 262 |
| Example 309 | | 1:3 | 2.08 | 172.3 | Green | 264 |
| Example 310 | 1-198:2-28 | 1:1 | 2.11 | 177.6 | Green | 273 |
| Example 311 | | 1:2 | 2.13 | 175.5 | Green | 275 |
| Example 312 | | 1:3 | 2.19 | 174.3 | Green | 277 |
| Example 313 | 1-197:2-32 | 1:1 | 2.32 | 179.7 | Green | 269 |
| Example 314 | | 1:2 | 2.37 | 178.4 | Green | 271 |
| Example 315 | | 1:3 | 2.39 | 177.3 | Green | 275 |
| Example 316 | 1-198:2-89 | 1:1 | 2.42 | 180.5 | Green | 283 |
| Example 317 | | 1:2 | 2.44 | 179.6 | Green | 288 |
| Example 318 | | 1:3 | 2.49 | 178.1 | Green | 291 |
| Example 319 | 1-201:2-95 | 1:1 | 2.13 | 175.6 | Green | 267 |
| Example 320 | | 1:2 | 2.19 | 174.3 | Green | 269 |
| Example 321 | | 1:3 | 2.21 | 173.8 | Green | 273 |
| Example 322 | 1-205:2-82 | 1:1 | 2.08 | 177.5 | Green | 264 |
| Example 323 | | 1:2 | 2.13 | 176.2 | Green | 266 |
| Example 324 | | 1:3 | 2.16 | 175.8 | Green | 269 |
| Example 325 | 1-208:2-95 | 1:1 | 2.15 | 165.2 | Green | 264 |
| Example 326 | | 1:2 | 2.18 | 163.8 | Green | 268 |
| Example 327 | | 1:3 | 2.20 | 161.2 | Green | 270 |
| Example 328 | 1-209:3-53 | 1:1 | 2.23 | 164.9 | Green | 269 |
| Example 329 | | 1:2 | 2.26 | 163.2 | Green | 273 |
| Example 330 | | 1:3 | 2.32 | 162.6 | Green | 275 |
| Example 331 | 1-210:3-53 | 1:1 | 2.15 | 178.3 | Green | 266 |
| Example 332 | | 1:2 | 2.19 | 177.6 | Green | 268 |
| Example 333 | | 1:3 | 2.23 | 176.5 | Green | 271 |
| Example 334 | 1-213:2-94 | 1:1 | 2.34 | 174.2 | Green | 282 |
| Example 335 | | 1:2 | 2.35 | 173.5 | Green | 286 |
| Example 336 | | 1:3 | 2.39 | 171.6 | Green | 289 |
| Example 337 | 1-216:2-94 | 1:1 | 2.41 | 180.5 | Green | 277 |
| Example 338 | | 1:2 | 2.44 | 179.6 | Green | 279 |
| Example 339 | | 1:3 | 2.53 | 178.5 | Green | 281 |
| Example 340 | 1-217:2-95 | 1:1 | 2.08 | 174.6 | Green | 285 |
| Example 341 | | 1:2 | 2.16 | 173.2 | Green | 291 |
| Example 342 | | 1:3 | 2.23 | 172.5 | Green | 299 |
| Example 343 | 1-219:2-82 | 1:1 | 2.23 | 177.4 | Green | 271 |
| Example 344 | | 1:2 | 2.35 | 176.2 | Green | 275 |
| Example 345 | | 1:3 | 2.41 | 175.8 | Green | 279 |
| Example 346 | 1-222:2-28 | 1:1 | 2.33 | 179.4 | Green | 280 |
| Example 347 | | 1:2 | 2.39 | 178.1 | Green | 283 |
| Example 348 | | 1:3 | 2.44 | 177.5 | Green | 288 |
| Example 349 | 1-224:3-4 | 1:1 | 2.32 | 168.7 | Green | 274 |
| Example 350 | | 1:2 | 2.37 | 167.4 | Green | 277 |
| Example 351 | | 1:3 | 2.42 | 166.5 | Green | 279 |
| Example 352 | 1-226:3-32 | 1:1 | 2.16 | 167.4 | Green | 281 |
| Example 353 | | 1:2 | 2.23 | 166.3 | Green | 283 |
| Example 354 | | 1:3 | 2.37 | 165.2 | Green | 288 |
| Example 355 | 1-227:3-53 | 1:1 | 2.08 | 179.4 | Green | 276 |
| Example 356 | | 1:2 | 2.17 | 178.2 | Green | 279 |
| Example 357 | | 1:3 | 2.25 | 177.6 | Green | 281 |
| Example 358 | 1-232:3-53 | 1:1 | 2.09 | 175.4 | Green | 283 |
| Example 359 | | 1:2 | 2.13 | 174.9 | Green | 288 |
| Example 360 | | 1:3 | 2.16 | 173.1 | Green | 290 |
| Example 361 | 1-233:2-92 | 1:1 | 2.44 | 168.5 | Green | 292 |
| Example 362 | | 1:2 | 2.52 | 167.2 | Green | 296 |
| Example 363 | | 1:3 | 2.56 | 166.4 | Green | 300 |
| Example 364 | 1-236:2-89 | 1:1 | 2.38 | 174.9 | Green | 284 |
| Example 365 | | 1:2 | 2.41 | 173.4 | Green | 288 |
| Example 366 | | 1:3 | 2.46 | 172.6 | Green | 289 |
| Example 367 | 1-241:2-95 | 1:1 | 2.31 | 177.8 | Green | 271 |
| Example 368 | | 1:2 | 2.37 | 176.2 | Green | 276 |
| Example 369 | | 1:3 | 2.40 | 175.1 | Green | 279 |
| Example 370 | 1-245:3-53 | 1:1 | 2.33 | 169.5 | Green | 261 |
| Example 371 | | 1:2 | 2.38 | 168.2 | Green | 265 |
| Example 372 | | 1:3 | 2.43 | 167.4 | Green | 269 |
| Example 373 | 1-247:3-12 | 1:1 | 2.26 | 168.7 | Green | 274 |
| Example 374 | | 1:2 | 2.32 | 167.2 | Green | 277 |
| Example 375 | | 1:3 | 2.39 | 166.8 | Green | 283 |
| Example 376 | 1-248:3-32 | 1:1 | 2.46 | 169.5 | Green | 271 |
| Example 377 | | 1:2 | 2.47 | 168.2 | Green | 276 |
| Example 378 | | 1:3 | 2.49 | 167.5 | Green | 278 |
| Example 379 | 1-249:2-95 | 1:1 | 2.60 | 132.4 | Green | 200 |
| Example 380 | | 1:2 | 2.61 | 131.6 | Green | 203 |
| Example 381 | | 1:3 | 2.66 | 130.8 | Green | 209 |
| Example 382 | 1-251:2-94 | 1:1 | 2.64 | 131.8 | Green | 213 |
| Example 383 | | 1:2 | 2.65 | 131.2 | Green | 218 |
| Example 384 | | 1:3 | 2.69 | 130.5 | Green | 220 |
| Example 385 | 1-253:2-82 | 1:1 | 2.87 | 139.7 | Green | 206 |
| Example 386 | | 1:2 | 2.91 | 138.5 | Green | 211 |
| Example 387 | | 1:3 | 2.93 | 137.1 | Green | 216 |
| Example 388 | 1-256:2-95 | 1:1 | 2.74 | 135.2 | Green | 205 |
| Example 389 | | 1:2 | 2.76 | 134.9 | Green | 209 |
| Example 390 | | 1:3 | 2.77 | 134.1 | Green | 213 |
| Example 391 | 1-257:2-28 | 1:1 | 2.64 | 132.8 | Green | 208 |
| Example 392 | | 1:2 | 2.66 | 131.5 | Green | 213 |
| Example 393 | | 1:3 | 2.73 | 130.7 | Green | 220 |
| Example 394 | 1-260:2-32 | 1:1 | 2.87 | 129.5 | Green | 200 |
| Example 395 | | 1:2 | 2.89 | 128.4 | Green | 209 |
| Example 396 | | 1:3 | 2.90 | 127.3 | Green | 211 |
| Example 397 | 1-263:2-32 | 1:1 | 2.88 | 128.5 | Green | 205 |
| Example 398 | | 1:2 | 2.93 | 127.6 | Green | 209 |
| Example 399 | | 1:3 | 2.95 | 126.5 | Green | 213 |
| Example 400 | 1-266:2-4 | 1:1 | 2.74 | 129.3 | Green | 210 |
| Example 401 | | 1:2 | 2.78 | 128.5 | Green | 212 |
| Example 402 | | 1:3 | 2.80 | 127.7 | Green | 216 |
| Example 403 | 1-270:2-92 | 1:1 | 2.74 | 125.8 | Green | 220 |
| Example 404 | | 1:2 | 2.79 | 124.9 | Green | 222 |
| Example 405 | | 1:3 | 2.83 | 123.2 | Green | 223 |
| Example 406 | 1-273:3-26 | 1:1 | 2.77 | 132.5 | Green | 221 |
| Example 407 | | 1:2 | 2.83 | 131.8 | Green | 225 |
| Example 408 | | 1:3 | 2.86 | 130.5 | Green | 226 |
| Example 409 | 1-274:3-53 | 1:1 | 2.65 | 140.2 | Green | 220 |
| Example 410 | | 1:2 | 2.69 | 138.5 | Green | 223 |
| Example 411 | | 1:3 | 2.71 | 137.2 | Green | 225 |
| Example 412 | 1-276:3-86 | 1:1 | 2.66 | 135.4 | Green | 206 |
| Example 413 | | 1:2 | 2.69 | 134.8 | Green | 213 |
| Example 414 | | 1:3 | 2.71 | 132.9 | Green | 219 |
| Example 415 | 1-279:3-116 | 1:1 | 2.68 | 129.7 | Green | 200 |
| Example 416 | | 1:2 | 2.70 | 128.5 | Green | 208 |
| Example 417 | | 1:3 | 2.73 | 127.4 | Green | 213 |
| Example 418 | 1-280:3-94 | 1:1 | 2.89 | 131.6 | Green | 211 |
| Example 419 | | 1:2 | 2.94 | 130.5 | Green | 216 |
| Example 420 | | 1:3 | 2.99 | 129.8 | Green | 221 |
| Example 421 | 1-287:3-94 | 1:1 | 2.87 | 132.2 | Green | 217 |
| Example 422 | | 1:2 | 2.93 | 131.5 | Green | 223 |
| Example 423 | | 1:3 | 2.95 | 130.8 | Green | 229 |
| Example 424 | 1-289:3-26 | 1:1 | 2.74 | 128.7 | Green | 201 |
| Example 425 | | 1:2 | 2.78 | 127.5 | Green | 203 |
| Example 426 | | 1:3 | 2.83 | 126.4 | Green | 208 |
| Example 427 | 1-296:3-86 | 1:1 | 2.69 | 121.9 | Green | 204 |
| Example 428 | | 1:2 | 2.74 | 121.1 | Green | 208 |
| Example 429 | | 1:3 | 2.77 | 120.8 | Green | 210 |
| Example 430 | 1-297:3-86 | 1:1 | 2.68 | 132.6 | Green | 211 |
| Example 431 | | 1:2 | 2.71 | 131.2 | Green | 219 |
| Example 432 | | 1:3 | 2.75 | 130.5 | Green | 220 |
| Example 433 | 1-300:3-112 | 1:1 | 2.82 | 137.4 | Green | 205 |
| Example 434 | | 1:2 | 2.88 | 136.5 | Green | 208 |
| Example 435 | | 1:3 | 2.91 | 135.3 | Green | 213 |
| Example 436 | 1-301:3-12 | 1:1 | 2.78 | 120.2 | Green | 180 |
| Example 437 | | 1:2 | 2.82 | 119.4 | Green | 183 |
| Example 438 | | 1:3 | 2.86 | 118.7 | Green | 187 |
| Example 439 | 1-302:3-32 | 1:1 | 2.77 | 116.5 | Green | 185 |
| Example 440 | | 1:2 | 2.83 | 115.8 | Green | 188 |
| Example 441 | | 1:3 | 2.88 | 114.2 | Green | 193 |
| Example 442 | 1-305:3-53 | 1:1 | 2.89 | 119.5 | Green | 181 |
| Example 443 | | 1:2 | 2.91 | 117.4 | Green | 183 |
| Example 444 | | 1:3 | 2.95 | 116.1 | Green | 188 |
| Example 445 | 1-310:3-86 | 1:1 | 2.77 | 120.5 | Green | 190 |
| Example 446 | | 1:2 | 2.79 | 118.7 | Green | 192 |
| Example 447 | | 1:3 | 2.83 | 117.3 | Green | 195 |
| Example 448 | 1-312:3-94 | 1:1 | 2.83 | 100.5 | Green | 189 |
| Example 449 | | 1:2 | 2.85 | 99.4 | Green | 191 |
| Example 450 | | 1:3 | 2.90 | 98.7 | Green | 194 |
| Example 451 | 1-313:3-80 | 1:1 | 2.78 | 99.5 | Green | 198 |
| Example 452 | | 1:2 | 2.82 | 98.1 | Green | 200 |
| Example 453 | | 1:3 | 2.88 | 97.3 | Green | 201 |
| Example 454 | 1-316:3-86 | 1:1 | 2.71 | 96.5 | Green | 187 |
| Example 455 | | 1:2 | 2.75 | 95.1 | Green | 190 |
| Example 456 | | 1:3 | 2.80 | 94.8 | Green | 194 |
| Example 457 | 1-321:3-12 | 1:1 | 2.63 | 110.5 | Green | 182 |
| Example 458 | | 1:2 | 2.65 | 109.4 | Green | 188 |
| Example 459 | | 1:3 | 2.69 | 107.3 | Green | 193 |
| Example 460 | 1-324:3-53 | 1:1 | 2.77 | 95.2 | Green | 182 |
| Example 461 | | 1:2 | 2.79 | 94.7 | Green | 185 |
| Example 462 | | 1:3 | 2.83 | 93.1 | Green | 194 |
| Example 463 | 1-326:3-86 | 1:1 | 2.69 | 92.8 | Green | 187 |
| Example 464 | | 1:2 | 2.72 | 91.6 | Green | 194 |
| Example 465 | | 1:3 | 2.77 | 90.7 | Green | 199 |
| Example 466 | 1-330:2-89 | 1:1 | 2.68 | 110.6 | Green | 182 |
| Example 467 | | 1:2 | 2.71 | 109.4 | Green | 186 |
| Example 468 | | 1:3 | 2.74 | 108.5 | Green | 191 |
| Example 469 | 1-333:2-95 | 1:1 | 2.72 | 111.6 | Green | 180 |
| Example 470 | | 1:2 | 2.77 | 110.3 | Green | 182 |
| Example 471 | | 1:3 | 2.83 | 109.7 | Green | 187 |
| Example 472 | 1-337:2-82 | 1:1 | 2.63 | 112.6 | Green | 180 |
| Example 473 | | 1:2 | 2.69 | 110.8 | Green | 183 |
| Example 474 | | 1:3 | 2.70 | 107.7 | Green | 187 |
| Example 475 | 1-341:2-94 | 1:1 | 2.74 | 119.4 | Green | 190 |
| Example 476 | | 1:2 | 2.79 | 118.2 | Green | 193 |
| Example 477 | | 1:3 | 2.81 | 117.2 | Green | 199 |
| Example 478 | 1-347:2-82 | 1:1 | 2.85 | 116.5 | Green | 187 |
| Example 479 | | 1:2 | 2.89 | 110.3 | Green | 193 |
| Example 480 | | 1:3 | 2.94 | 109.7 | Green | 199 |
| Example 481 | 1-351:3-94 | 1:1 | 2.63 | 98.5 | Green | 172 |
| Example 482 | | 1:2 | 2.68 | 97.6 | Green | 177 |
| Example 483 | | 1:3 | 2.77 | 96.5 | Green | 183 |
| Example 484 | 1-352:3-116 | 1:1 | 2.60 | 99.4 | Green | 182 |
| Example 485 | | 1:2 | 2.62 | 98.1 | Green | 186 |
| Example 486 | | 1:3 | 2.66 | 97.5 | Green | 193 |
| Example 487 | 1-355:3-26 | 1:1 | 3.70 | 88.0 | Green | 140 |
| Example 488 | | 1:2 | 3.72 | 87.6 | Green | 143 |
| Example 489 | | 1:3 | 3.75 | 86.4 | Green | 149 |
| Example 490 | 1-356:3-80 | 1:1 | 3.82 | 85.6 | Green | 152 |
| Example 491 | | 1:2 | 3.85 | 84.7 | Green | 155 |
| Example 492 | | 1:3 | 3.88 | 83.1 | Green | 163 |
| Example 493 | 1-360:3-12 | 1:1 | 3.89 | 80.9 | Green | 148 |
| Example 494 | | 1:2 | 3.91 | 79.1 | Green | 152 |
| Example 495 | | 1:3 | 3.94 | 78.6 | Green | 159 |
| Example 496 | 1-364:3-26 | 1:1 | 3.72 | 77.9 | Green | 142 |
| Example 497 | | 1:2 | 3.74 | 76.4 | Green | 149 |
| Example 498 | | 1:3 | 3.78 | 75.1 | Green | 153 |
| Example 499 | 1-368:3-53 | 1:1 | 3.68 | 83.9 | Green | 144 |
| Example 500 | | 1:2 | 3.71 | 82.1 | Green | 148 |
| Example 501 | | 1:3 | 3.74 | 81.6 | Green | 150 |
| Example 502 | 1-372:3-86 | 1:1 | 3.78 | 85.7 | Green | 149 |
| Example 503 | | 1:2 | 3.82 | 84.2 | Green | 153 |
| Example 504 | | 1:3 | 3.85 | 83.9 | Green | 159 |
| Example 505 | 1-373:2-32 | 1:1 | 3.68 | 80.7 | Green | 151 |
| Example 506 | | 1:2 | 3.71 | 79.5 | Green | 155 |
| Example 507 | | 1:3 | 3.75 | 78.2 | Green | 160 |
| Example 508 | 1-379:2-28 | 1:1 | 3.81 | 78.4 | Green | 148 |
| Example 509 | | 1:2 | 3.84 | 77.6 | Green | 152 |
| Example 510 | | 1:3 | 3.87 | 76.8 | Green | 156 |
| Example 511 | 1-381:2-92 | 1:1 | 3.74 | 80.9 | Green | 159 |
| Example 512 | | 1:2 | 3.77 | 79.4 | Green | 160 |
| Example 513 | | 1:3 | 3.81 | 78.2 | Green | 162 |
| Example 514 | 1-388:2-89 | 1:1 | 3.69 | 83.4 | Green | 148 |
| Example 515 | | 1:2 | 3.70 | 82.6 | Green | 152 |
| Example 516 | | 1:3 | 3.77 | 81.8 | Green | 159 |
| Example 517 | 1-395:3-94 | 1:1 | 3.81 | 84.6 | Green | 151 |
| Example 518 | | 1:2 | 3.83 | 83.5 | Green | 153 |
| Example 519 | | 1:3 | 3.88 | 82.9 | Green | 158 |
| Example 520 | 1-400:3-86 | 1:1 | 3.85 | 87.4 | Green | 147 |
| Example 521 | | 1:2 | 3.89 | 86.2 | Green | 153 |
| Example 522 | | 1:3 | 3.92 | 84.1 | Green | 159 |
| Example 523 | 1-402:3-112 | 1:1 | 3.71 | 88.0 | Green | 152 |
| Example 524 | | 1:2 | 3.75 | 87.1 | Green | 155 |
| Example 525 | | 1:3 | 3.77 | 85.4 | Green | 157 |
| Example 526 | 1-408:3-94 | 1:1 | 3.79 | 87.6 | Green | 148 |
| Example 527 | | 1:2 | 3.82 | 86.2 | Green | 152 |
| Example 528 | | 1:3 | 3.85 | 85.1 | Green | 159 |

The comparative compounds used in Table 10 are the same as the comparative compounds of Table 9.

Comparing the results of Table 9 with the results of Table 10, it can be identified that the organic light emitting devices using the heterocyclic compound of Chemical Formula 1 and the heterocyclic compound of Chemical Formula 2 or Chemical Formula 3 as a light emitting layer host have significantly improved driving voltage, light emission efficiency and lifetime compared to the organic light emitting devices of Comparative Examples.

Effects of more superior efficiency and lifetime are obtained when the compound of Chemical Formula 1 and the compound of Chemical Formula 2 or Chemical Formula 3 are included at the same time. Such a result may lead to a forecast that an exciplex phenomenon occurs when the two compounds are included at the same time.

The exciplex phenomenon is a phenomenon of releasing energy having sizes of a donor (p-host) HOMO level and an acceptor (n-host) LUMO level due to electron exchanges between two molecules. When the exciplex phenomenon occurs between two molecules, reverse intersystem crossing (RISC) occurs, and as a result, internal quantum efficiency of fluorescence may increase up to 100%. When a donor (p-host) having a favorable hole transport ability and an acceptor (n-host) having a favorable electron transport ability are used as a host of a light emitting layer, holes are injected to the p-host and electrons are injected to the n-host, and therefore, a driving voltage may be lowered, which resultantly helps with enhancement in the lifetime. In the present disclosure, it was identified that excellent device properties were obtained when, as the light emitting layer host, using the compounds of Chemical Formula 2 and Chemical Formula 3 as a donor role and using the compound of Chemical Formula 1 as an acceptor role.

In charge transfer, the host material allows low driving, high efficiency and lifetime of the light emitting device through a stable structure.

Particularly, in the heterocyclic compound represented by Chemical Formula 1, the dibenzofuran parent structure

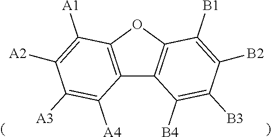

forms a resonance structure, and accordingly, the No. 2 and No. 4 carbon sites of the benzene ring included in the parent structure are negatively charged, and the No. 1 and No. 3 carbon sites become relatively electron deficient. Herein, when bonding an electron transport moiety to the No. 1 and No. 3 carbon sites that are relatively electron deficient and bonding a hole transport moiety to the No. 2 and No. 4 carbon sites that are relatively electron abundant, the transport speed of electrons in the molecule becomes faster. Accordingly, when a triazine substituent is introduced to the relatively electron-deficient No. 1 and No. 3 carbon sites of the benzene ring in the dibenzofuran parent structure as in the heterocyclic compound represented by Chemical Formula 1 according to the present disclosure, the role of the triazine substituent is strengthened compared to when a triazine substituent is introduced to the No. 2 and No. 4 carbon sites of the benzene ring.

In addition, the heterocyclic compound represented by Chemical Formula 1 according to the present disclosure further includes a dibenzofuran or dibenzothiophene substituent in the triazine substituent linked to the dibenzofuran parent structure, and further includes an aryl group or a heteroaryl group in the dibenzofuran or dibenzothiophene substituent, and accordingly, electron transport may be strengthened through expanding aromaticity of the LUMO (lowest unoccupied molecular orbital) level and the resonance structure. Particularly, when a heterocyclic substituent bonds to the dibenzofuran or dibenzothiophene substituent, the heterocyclic substituent includes an atom having higher electronegativity than carbon, and a property of donating electrons to the triazine substituent becomes weak compared to an aryl substituent. Accordingly, aromaticity of the LUMO becomes more superior when an aryl substituent bonds to the dibenzofuran or dibenzothiophene substituent compared to when a heterocyclic substituent bonds to the dibenzofuran or dibenzothiophene substituent, and electron transport in the heterocyclic compound represented by Chemical Formula 1 becomes smoother. When the heterocyclic compound represented by Chemical Formula 1 is used in an organic light emitting device together with the heterocyclic compound according to Chemical Formula 2 or 3 according to the present disclosure, there is an effect that intermolecular electron transport occurs more smoothly.

Furthermore, when substituting all hydrogens with deuterium in the structure of the heterocyclic compound represented by Chemical Formula 1, synthesis yield and deuterium substitution rate may decrease, and process efficiency may decrease since D6-benzene with a relatively high unit price is used in excess. Accordingly, intensively substituting hydrogens in the portion corresponding to the HOMO of the heterocyclic compound represented by the Chemical Formula 1 with deuterium may increase synthesis yield and deuterium substitution rate while using a relatively small amount of D6-benzene.

Accordingly, by using the compound represented by Chemical Formula 1 in the organic material layer, it is possible to lower a driving voltage of an organic light emitting device, enhance light emission efficiency, and enhance lifetime properties of an organic light emitting device due to thermal stability of the compound.

### REFERENCE NUMERAL

100: Substrate
200: Positive Electrode
300: Organic Material Layer
301: Hole Injection Layer
302: Hole Transport Layer
303: Light Emitting Layer
304: Hole Blocking Layer
305: Electron Transport Layer
306: Electron Injection Layer
400: Negative Electrode

1. A heterocyclic compound represented by the following Chemical Formula 1: 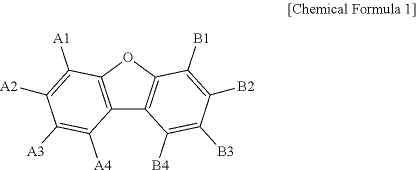
wherein, in Chemical Formula 1,
A1 to A4 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102 and —SiR101R102R103; and a group represented by the following Structural Formula 1, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group;
at least one of A1 to A4 is the group represented by the following Structural Formula 1; 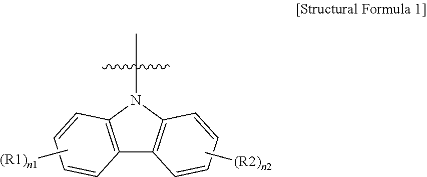
B1 to B4 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102 and —SiR101R102R103; and a group represented by the following Structural Formula 2, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group; and
at least one of B1 to B4 is the group represented by the following Structural Formula 2, 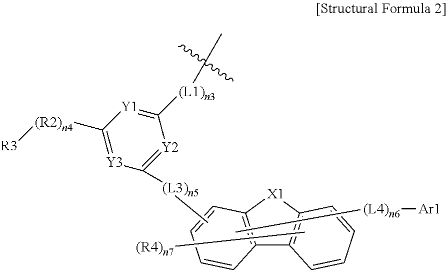
in Structural Formula 1,
R1 and R2 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102 and —SiR101R102R103, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group; and
n1 and n2 are the same as or different from each other, and each independently an integer of 0 to 4;
in Structural Formula 2,
X1 is O or S;
L1 to L4 are the same as or different from each other, and each independently a single bond; a substituted or unsubstituted C6 to C60 arylene group; or a substituted or unsubstituted C2 to C60 heteroarylene group;
Y1 to Y3 are the same as or different from each other and each independently CH or N, and at least one of Y1 to Y3 is N;
Ar1 is a substituted or unsubstituted C6 to C60 aryl group; or a substituted or unsubstituted C2 to C60 heteroaryl group;
R3 and R4 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102 and —SiR101R102R103, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group;
n3 to n6 are the same as or different from each other, and each independently an integer of 0 to 3; and
n7 is an integer of 0 to 6.

2. The heterocyclic compound of claim 1, wherein Chemical Formula 1 is represented by any one of the following Chemical Formula 1-1 or 1-2: 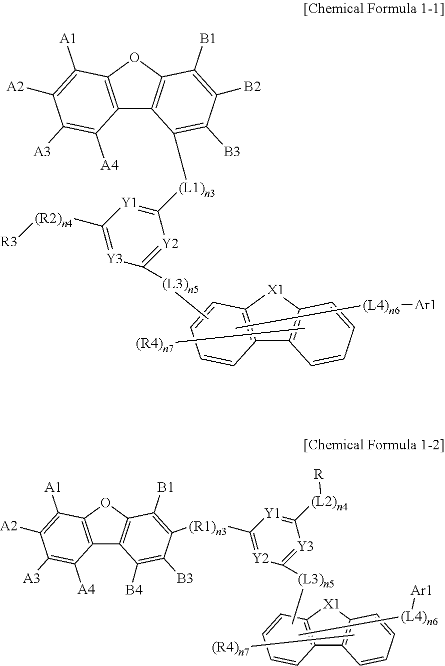
in Chemical Formulae 1-1 and 1-2,
A1 to A4, B1 to B4, X1, L1 to LA, Y1 to Y3, R3, R4, n3 to n7 and Ar1 have the same definitions as in Chemical Formula 1.

3. The heterocyclic compound of claim 1, wherein Structural Formula 2 is represented by any one of the following Structural Formulae 2-1 to 2-8: 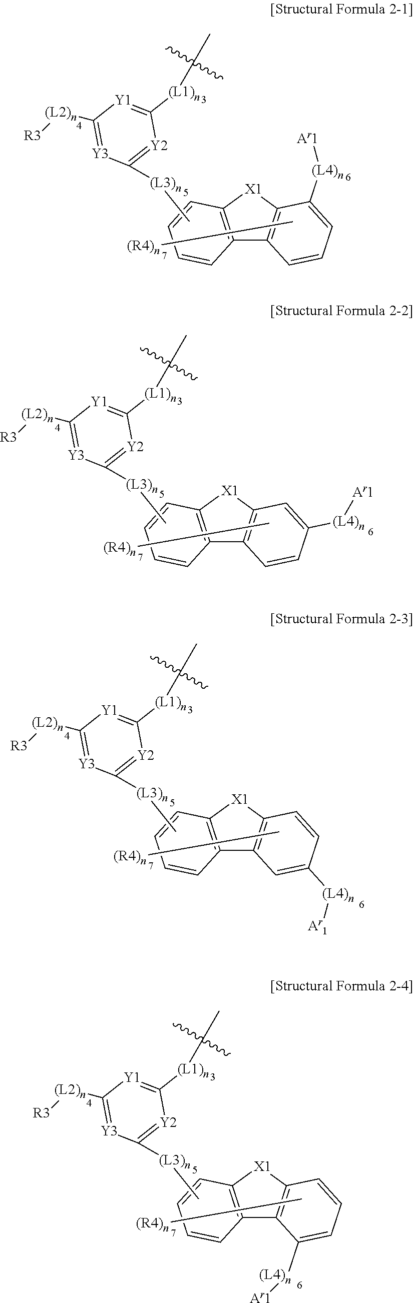 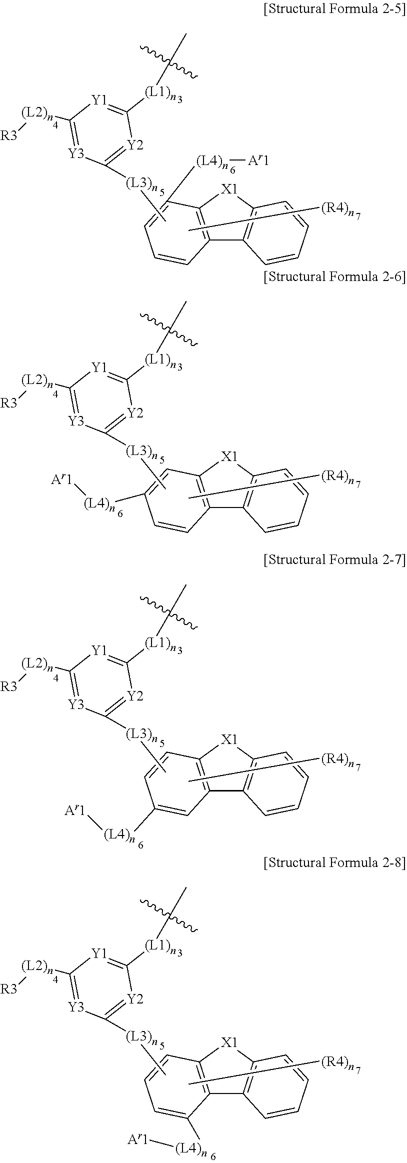
in Structural Formulae 2-1 to 2-8,
L1 to L4, X1, Y1 to Y3, R3, R4, n3 to n7 and Ar1 have the same definitions as in Chemical Formula 1.

4. The heterocyclic compound of claim 1, wherein the heterocyclic compound represented by Chemical Formula 1 does not include deuterium as a substituent, or has a deuterium content of 1% to 100% based on a total number of hydrogen atoms and deuterium atoms.

5. The heterocyclic compound of claim 1, wherein the heterocyclic compound represented by Chemical Formula 1 is represented by any one of the following compounds:

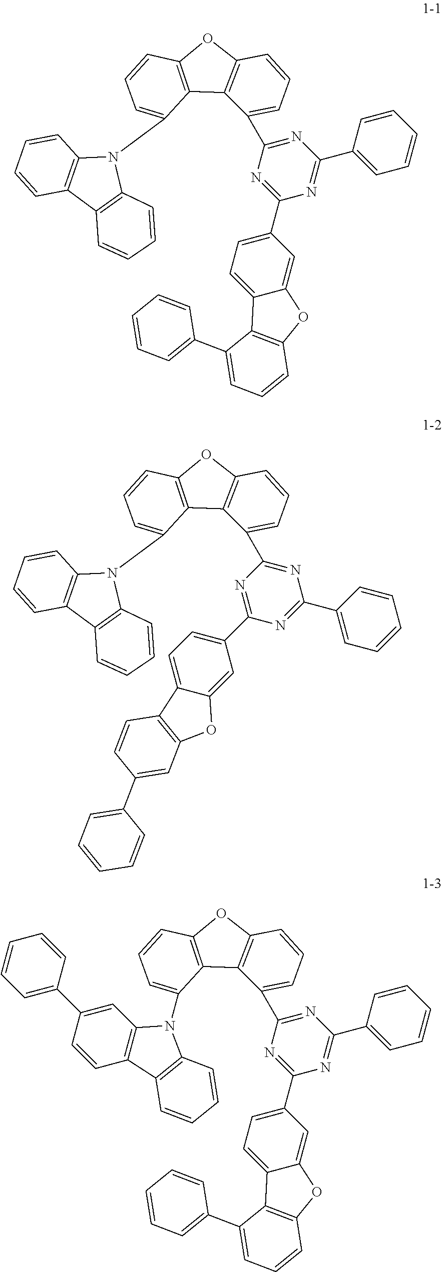 [SMILES: c1ccc(-c2nc(-c3ccc4c(c3)oc3cccc(-c5ccccc5)c34)nc(-c3cccc4oc5cccc(-n6c7ccccc7c7ccccc76)c5c34)n2)cc1 ; c1ccc(-c2ccc3c(c2)oc2cc(-c4nc(-c5ccccc5)nc(-c5cccc6oc7cccc(-n8c9ccccc9c9ccccc98)c7c56)n4)ccc23)cc1 ; c1ccc(-c2ccc3c4ccccc4n(-c4cccc5oc6cccc(-c7nc(-c8ccccc8)nc(-c8ccc9c(c8)oc8cccc(-c%10ccccc%10)c89)n7)c6c45)c3c2)cc1]

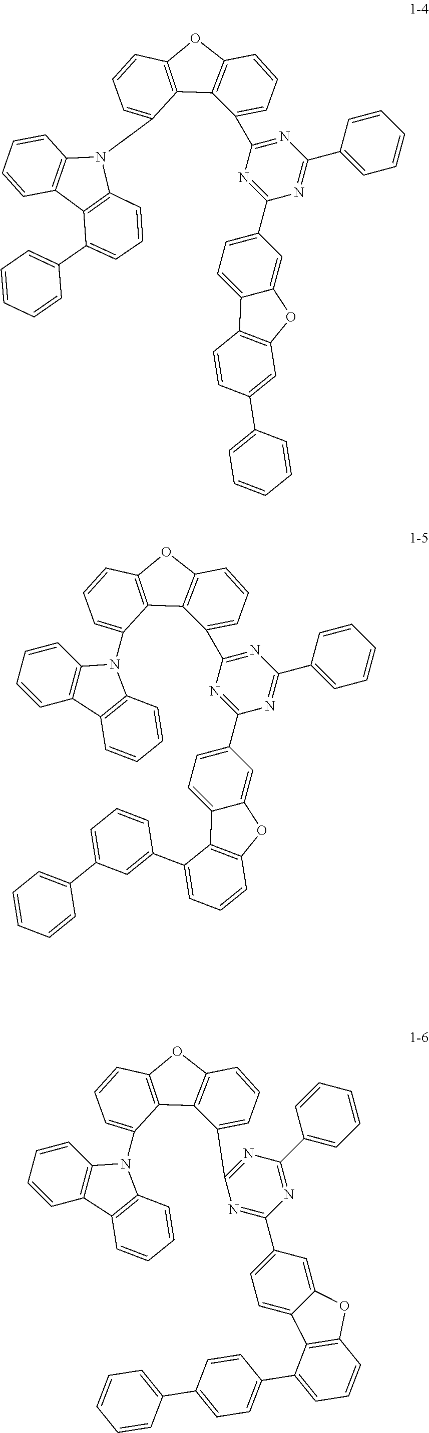 [SMILES: c1ccc(-c2ccc3c(c2)oc2cc(-c4nc(-c5ccccc5)nc(-c5cccc6oc7cccc(-n8c9ccccc9c9c(-c%10ccccc%10)cccc98)c7c56)n4)ccc23)cc1 ; c1ccc(-c2cccc(-c3cccc4oc5cc(-c6nc(-c7ccccc7)nc(-c7cccc8oc9cccc(-n%10c%11ccccc%11c%11ccccc%11%10)c9c78)n6)ccc5c34)c2)cc1 ; c1ccc(-c2ccc(-c3cccc4oc5cc(-c6nc(-c7ccccc7)nc(-c7cccc8oc9cccc(-n%10c%11ccccc%11c%11ccccc%11%10)c9c78)n6)ccc5c34)cc2)cc1]

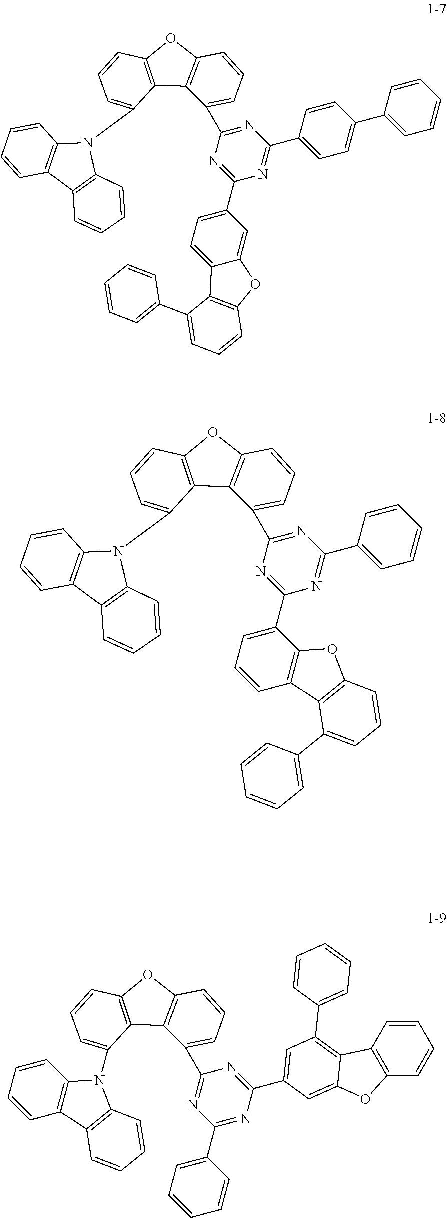 [SMILES: c1ccc(-c2ccc(-c3nc(-c4ccc5c(c4)oc4cccc(-c6ccccc6)c45)nc(-c4cccc5oc6cccc(-n7c8ccccc8c8ccccc87)c6c45)n3)cc2)cc1 ; c1ccc(-c2nc(-c3cccc4c3oc3cccc(-c5ccccc5)c34)nc(-c3cccc4oc5cccc(-n6c7ccccc7c7ccccc76)c5c34)n2)cc1 ; c1ccc(-c2nc(-c3cc(-c4ccccc4)c4c(c3)oc3ccccc34)nc(-c3cccc4oc5cccc(-n6c7ccccc7c7ccccc76)c5c34)n2)cc1]

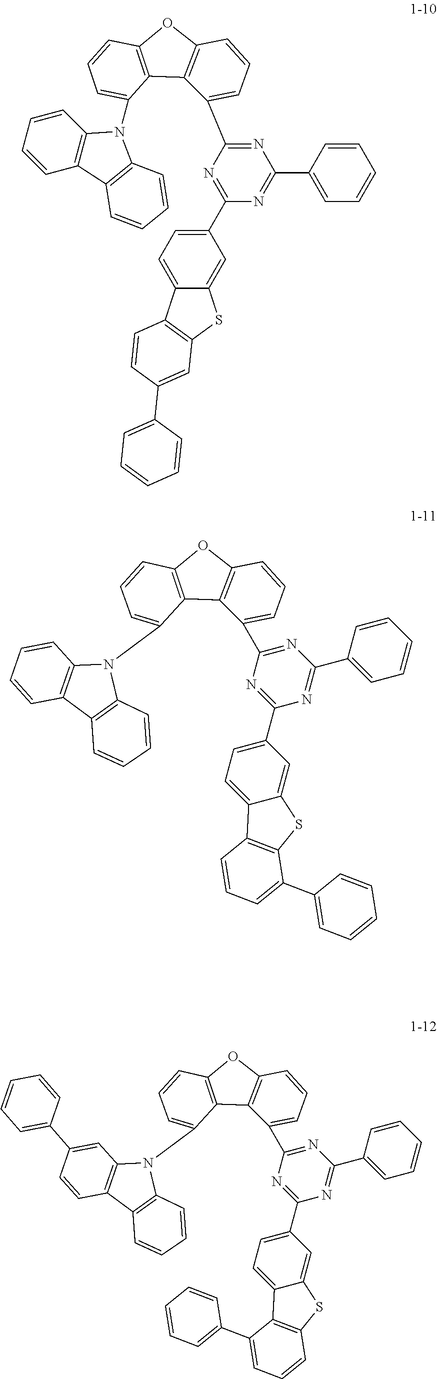 [SMILES: c1ccc(-c2ccc3c(c2)sc2cc(-c4nc(-c5ccccc5)nc(-c5cccc6oc7cccc(-n8c9ccccc9c9ccccc98)c7c56)n4)ccc23)cc1 ; c1ccc(-c2nc(-c3ccc4c(c3)sc3c(-c5ccccc5)cccc34)nc(-c3cccc4oc5cccc(-n6c7ccccc7c7ccccc76)c5c34)n2)cc1 ; c1ccc(-c2ccc3c4ccccc4n(-c4cccc5oc6cccc(-c7nc(-c8ccccc8)nc(-c8ccc9c(c8)sc8cccc(-c%10ccccc%10)c89)n7)c6c45)c3c2)cc1]

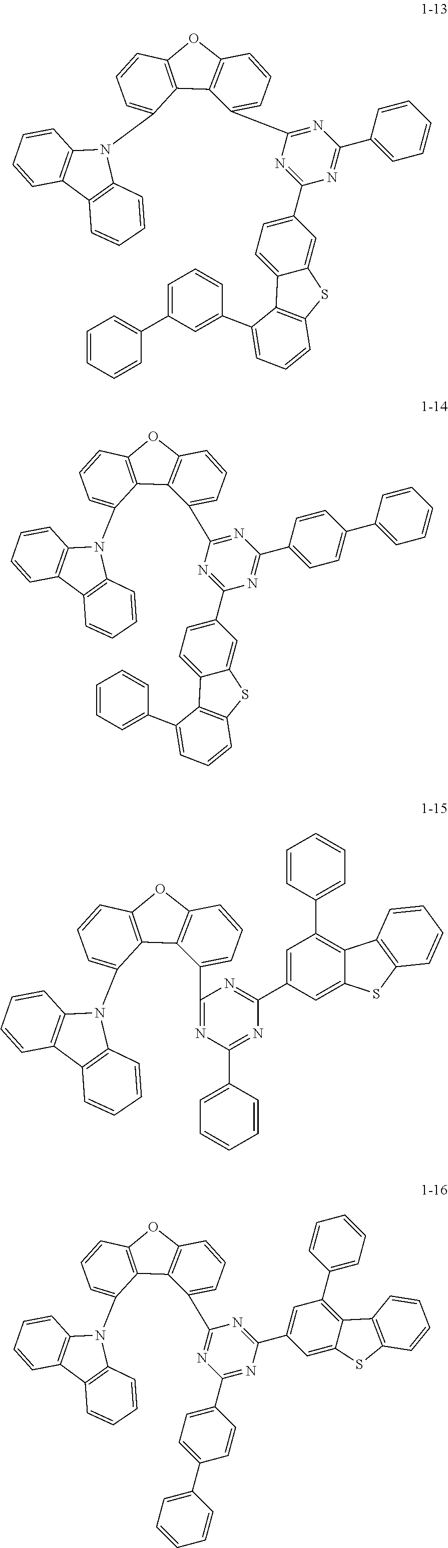 [SMILES: c1ccc(-c2cccc(-c3cccc4sc5cc(-c6nc(-c7ccccc7)nc(-c7cccc8oc9cccc(-n%10c%11ccccc%11c%11ccccc%11%10)c9c78)n6)ccc5c34)c2)cc1 ; c1ccc(-c2ccc(-c3nc(-c4ccc5c(c4)sc4cccc(-c6ccccc6)c45)nc(-c4cccc5oc6cccc(-n7c8ccccc8c8ccccc87)c6c45)n3)cc2)cc1 ; c1ccc(-c2nc(-c3cc(-c4ccccc4)c4c(c3)sc3ccccc34)nc(-c3cccc4oc5cccc(-n6c7ccccc7c7ccccc76)c5c34)n2)cc1 ; c1ccc(-c2ccc(-c3nc(-c4cc(-c5ccccc5)c5c(c4)sc4ccccc45)nc(-c4cccc5oc6cccc(-n7c8ccccc8c8ccccc87)c6c45)n3)cc2)cc1]

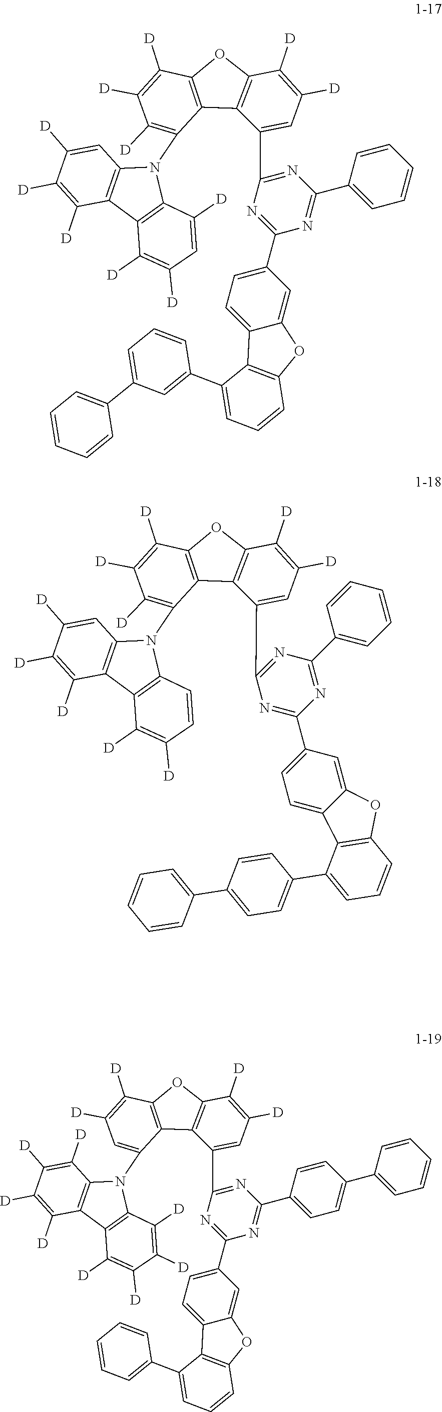

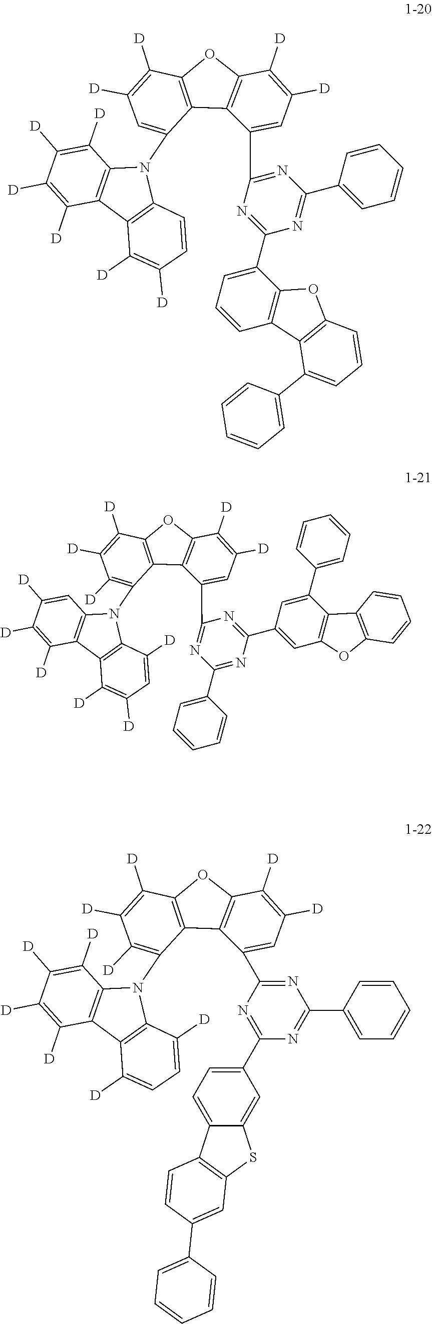 [SMILES: [2H]c1cc(-c2nc(-c3ccccc3)nc(-c3cccc4c3oc3cccc(-c5ccccc5)c34)n2)c2c(oc3c([2H])c([2H])cc(-n4c5ccc([2H])c([2H])c5c5c([2H])c([2H])c([2H])c([2H])c54)c32)c1[2H] ; [2H]c1cc2c(c([2H])c1[2H])c1c([2H])c([2H])cc([2H])c1n2-c1c([2H])c([2H])c([2H])c2oc3c([2H])c([2H])cc(-c4nc(-c5ccccc5)nc(-c5cc(-c6ccccc6)c6c(c5)oc5ccccc56)n4)c3c12 ; [2H]c1cc(-c2nc(-c3ccccc3)nc(-c3ccc4c(c3)sc3cc(-c5ccccc5)ccc34)n2)c2c(oc3c([2H])c([2H])c([2H])c(-n4c5c([2H])ccc([2H])c5c5c([2H])c([2H])c([2H])c([2H])c54)c32)c1[2H]]

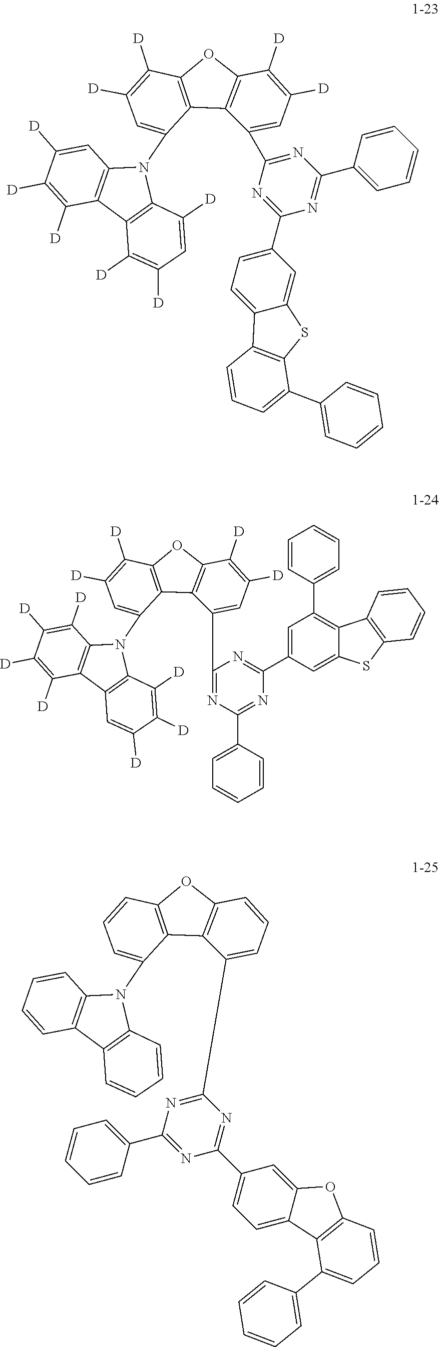 [SMILES: [2H]c1cc2c(c([2H])c1[2H])c1c([2H])c([2H])cc([2H])c1n2-c1cc([2H])c([2H])c2oc3c([2H])c([2H])cc(-c4nc(-c5ccccc5)nc(-c5ccc6c(c5)sc5c(-c7ccccc7)cccc56)n4)c3c12 ; [2H]c1cc2c3c([2H])c([2H])c([2H])c([2H])c3n(-c3cc([2H])c([2H])c4oc5c([2H])c([2H])cc(-c6nc(-c7ccccc7)nc(-c7cc(-c8ccccc8)c8c(c7)sc7ccccc78)n6)c5c34)c2c([2H])c1[2H] ; c1ccc(-c2nc(-c3ccc4c(c3)oc3cccc(-c5ccccc5)c34)nc(-c3cccc4oc5cccc(-n6c7ccccc7c7ccccc76)c5c34)n2)cc1]

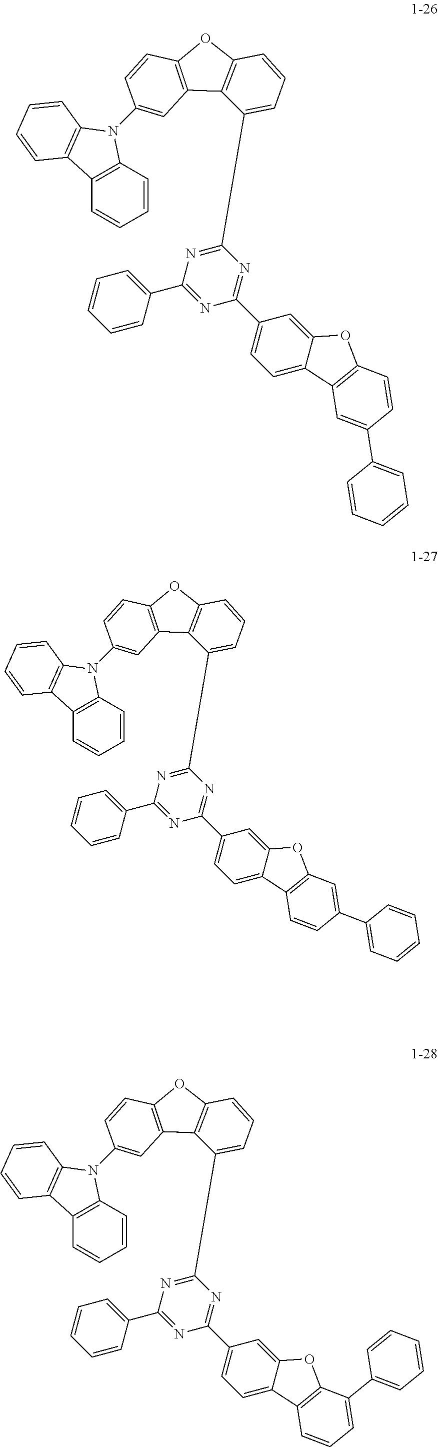 [SMILES: c1ccc(-c2ccc3oc4cc(-c5nc(-c6ccccc6)nc(-c6cccc7oc8ccc(-n9c%10ccccc%10c%10ccccc%109)cc8c67)n5)ccc4c3c2)cc1 ; c1ccc(-c2ccc3c(c2)oc2cc(-c4nc(-c5ccccc5)nc(-c5cccc6oc7ccc(-n8c9ccccc9c9ccccc98)cc7c56)n4)ccc23)cc1 ; c1ccc(-c2nc(-c3ccc4c(c3)oc3c(-c5ccccc5)cccc34)nc(-c3cccc4oc5ccc(-n6c7ccccc7c7ccccc76)cc5c34)n2)cc1]

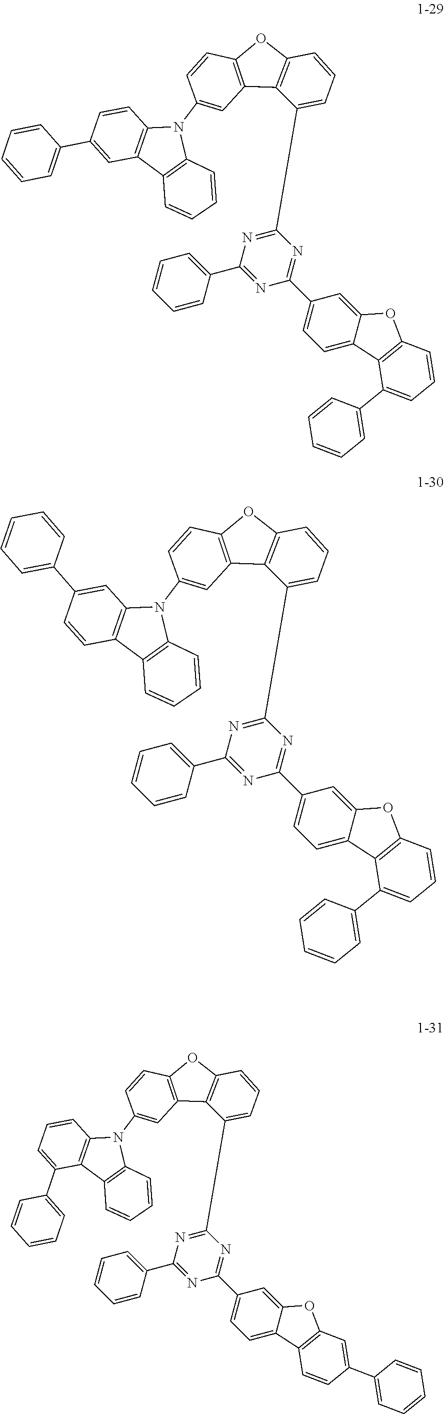 [SMILES: c1ccc(-c2ccc3c(c2)c2ccccc2n3-c2ccc3oc4cccc(-c5nc(-c6ccccc6)nc(-c6ccc7c(c6)oc6cccc(-c8ccccc8)c67)n5)c4c3c2)cc1 ; c1ccc(-c2ccc3c4ccccc4n(-c4ccc5oc6cccc(-c7nc(-c8ccccc8)nc(-c8ccc9c(c8)oc8cccc(-c%10ccccc%10)c89)n7)c6c5c4)c3c2)cc1 ; c1ccc(-c2ccc3c(c2)oc2cc(-c4nc(-c5ccccc5)nc(-c5cccc6oc7ccc(-n8c9ccccc9c9c(-c%10ccccc%10)cccc98)cc7c56)n4)ccc23)cc1]

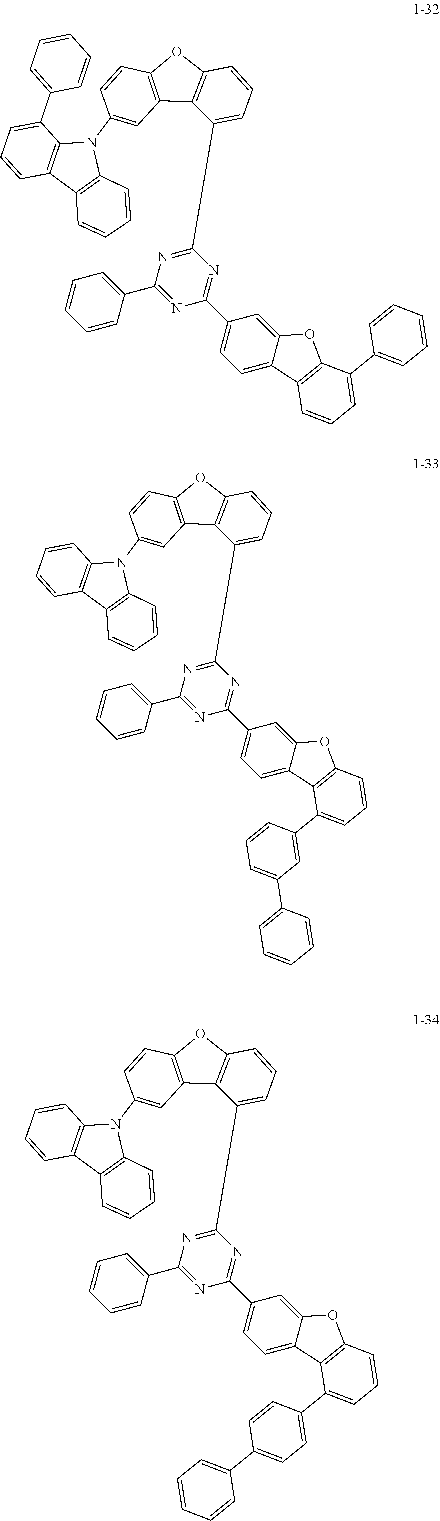 [SMILES: c1ccc(-c2nc(-c3ccc4c(c3)oc3c(-c5ccccc5)cccc34)nc(-c3cccc4oc5ccc(-n6c7ccccc7c7cccc(-c8ccccc8)c76)cc5c34)n2)cc1 ; c1ccc(-c2cccc(-c3cccc4oc5cc(-c6nc(-c7ccccc7)nc(-c7cccc8oc9ccc(-n%10c%11ccccc%11c%11ccccc%11%10)cc9c78)n6)ccc5c34)c2)cc1 ; c1ccc(-c2ccc(-c3cccc4oc5cc(-c6nc(-c7ccccc7)nc(-c7cccc8oc9ccc(-n%10c%11ccccc%11c%11ccccc%11%10)cc9c78)n6)ccc5c34)cc2)cc1]

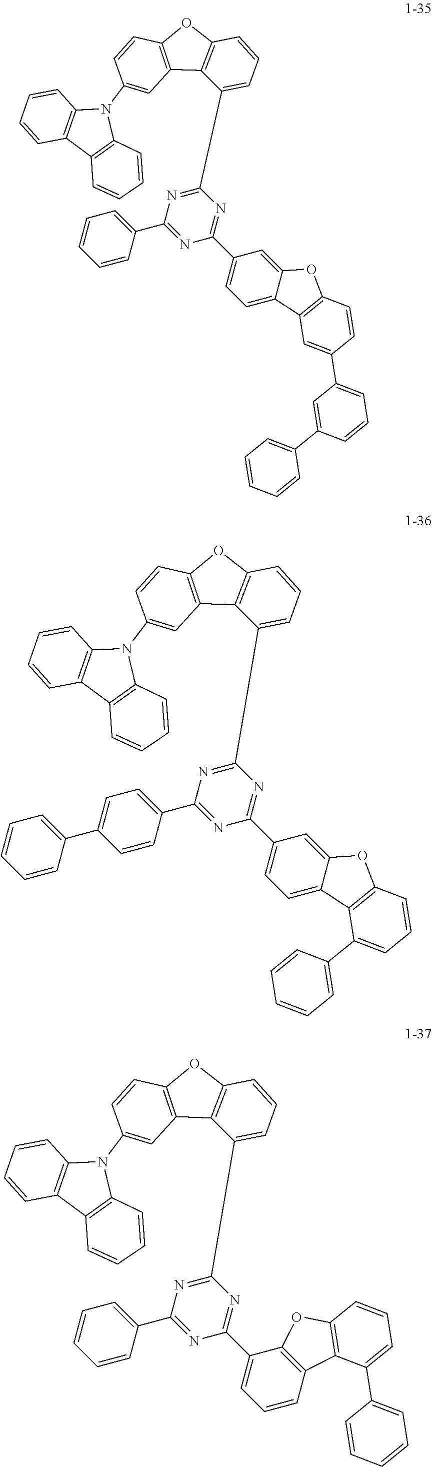 [SMILES: c1ccc(-c2cccc(-c3ccc4oc5cc(-c6nc(-c7ccccc7)nc(-c7cccc8oc9ccc(-n%10c%11ccccc%11c%11ccccc%11%10)cc9c78)n6)ccc5c4c3)c2)cc1 ; c1ccc(-c2ccc(-c3nc(-c4ccc5c(c4)oc4cccc(-c6ccccc6)c45)nc(-c4cccc5oc6ccc(-n7c8ccccc8c8ccccc87)cc6c45)n3)cc2)cc1 ; c1ccc(-c2nc(-c3cccc4c3oc3cccc(-c5ccccc5)c34)nc(-c3cccc4oc5ccc(-n6c7ccccc7c7ccccc76)cc5c34)n2)cc1]

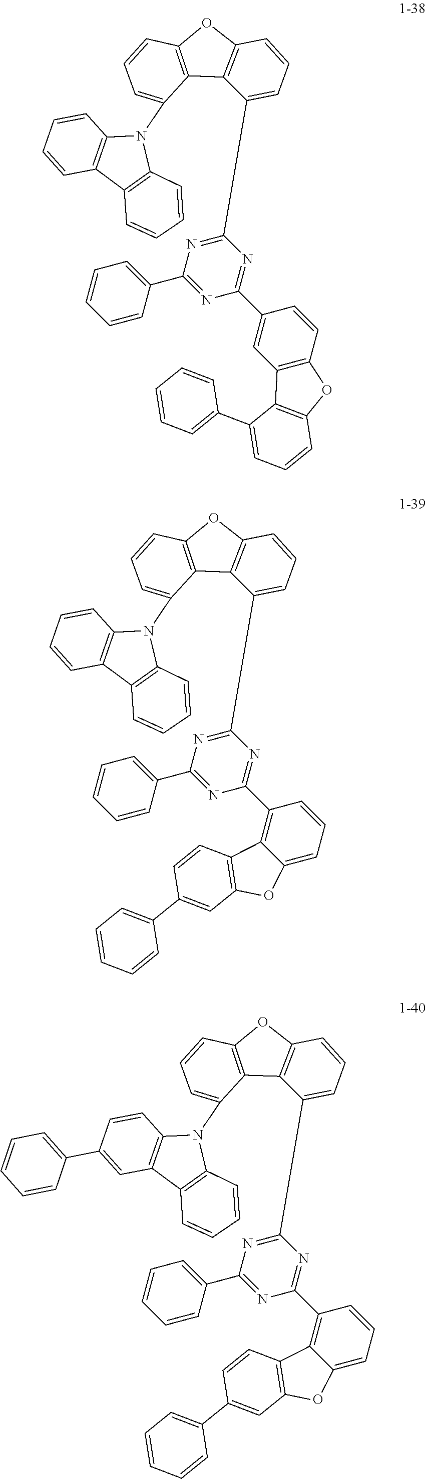 [SMILES: c1ccc(-c2nc(-c3ccc4oc5cccc(-c6ccccc6)c5c4c3)nc(-c3cccc4oc5cccc(-n6c7ccccc7c7ccccc76)c5c34)n2)cc1 ; c1ccc(-c2ccc3c(c2)oc2cccc(-c4nc(-c5ccccc5)nc(-c5cccc6oc7cccc(-n8c9ccccc9c9ccccc98)c7c56)n4)c23)cc1 ; c1ccc(-c2ccc3c(c2)oc2cccc(-c4nc(-c5ccccc5)nc(-c5cccc6oc7cccc(-n8c9ccccc9c9cc(-c%10ccccc%10)ccc98)c7c56)n4)c23)cc1]

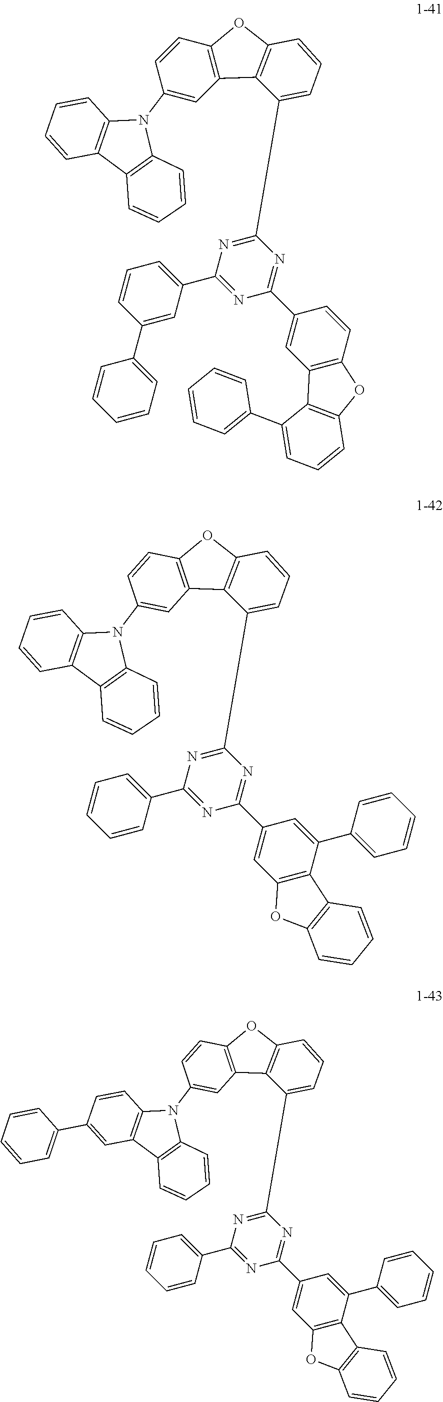 [SMILES: c1ccc(-c2cccc(-c3nc(-c4ccc5oc6cccc(-c7ccccc7)c6c5c4)nc(-c4cccc5oc6ccc(-n7c8ccccc8c8ccccc87)cc6c45)n3)c2)cc1 ; c1ccc(-c2nc(-c3cc(-c4ccccc4)c4c(c3)oc3ccccc34)nc(-c3cccc4oc5ccc(-n6c7ccccc7c7ccccc76)cc5c34)n2)cc1 ; c1ccc(-c2ccc3c(c2)c2ccccc2n3-c2ccc3oc4cccc(-c5nc(-c6ccccc6)nc(-c6cc(-c7ccccc7)c7c(c6)oc6ccccc67)n5)c4c3c2)cc1]

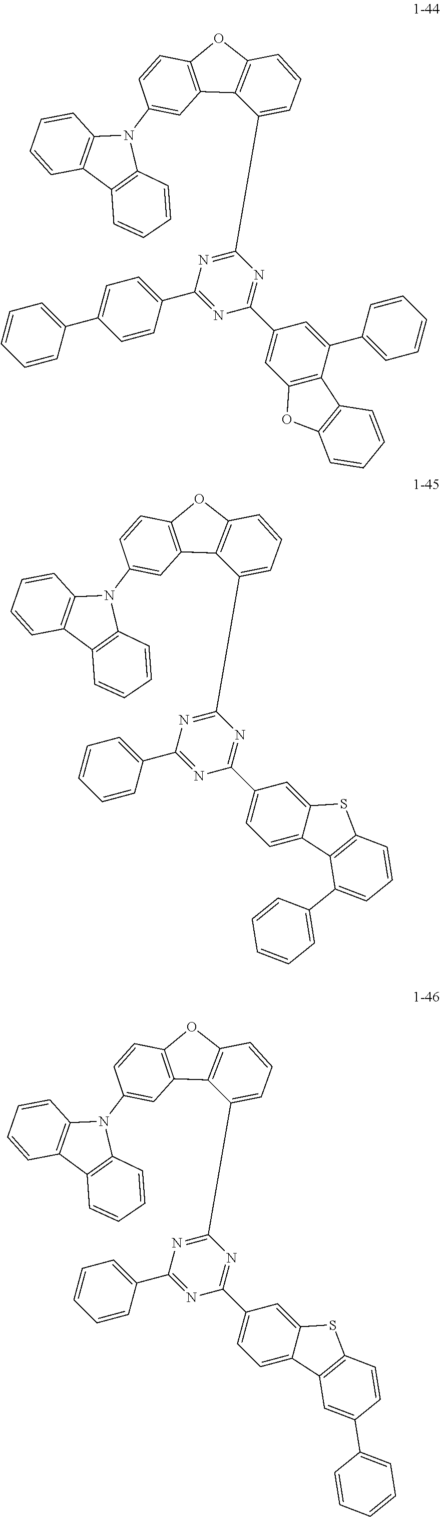 [SMILES: c1ccc(-c2ccc(-c3nc(-c4cc(-c5ccccc5)c5c(c4)oc4ccccc45)nc(-c4cccc5oc6ccc(-n7c8ccccc8c8ccccc87)cc6c45)n3)cc2)cc1 ; c1ccc(-c2nc(-c3ccc4c(c3)sc3cccc(-c5ccccc5)c34)nc(-c3cccc4oc5ccc(-n6c7ccccc7c7ccccc76)cc5c34)n2)cc1 ; c1ccc(-c2ccc3sc4cc(-c5nc(-c6ccccc6)nc(-c6cccc7oc8ccc(-n9c%10ccccc%10c%10ccccc%109)cc8c67)n5)ccc4c3c2)cc1]

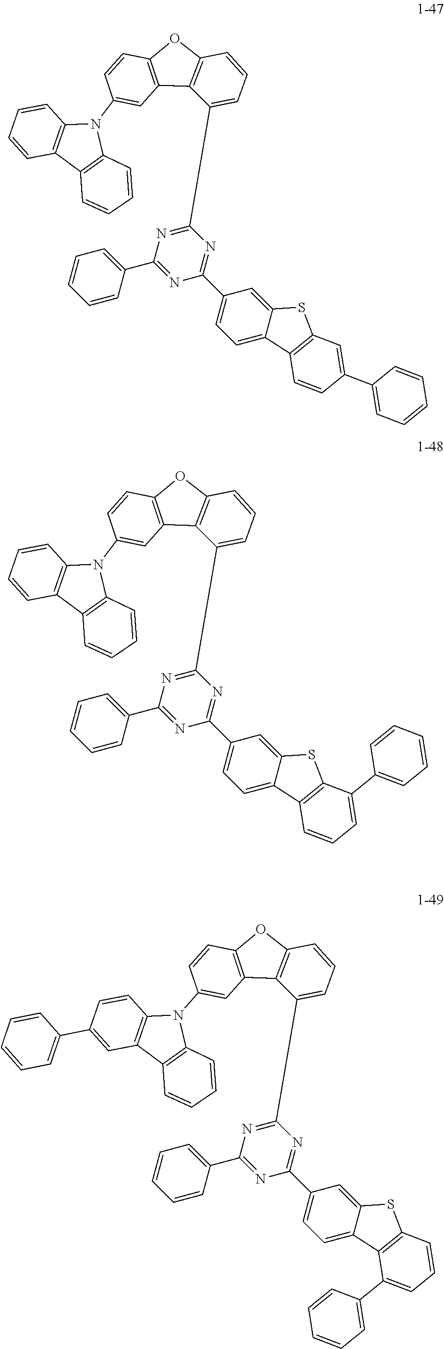 [SMILES: c1ccc(-c2ccc3c(c2)sc2cc(-c4nc(-c5ccccc5)nc(-c5cccc6oc7ccc(-n8c9ccccc9c9ccccc98)cc7c56)n4)ccc23)cc1 ; c1ccc(-c2nc(-c3ccc4c(c3)sc3c(-c5ccccc5)cccc34)nc(-c3cccc4oc5ccc(-n6c7ccccc7c7ccccc76)cc5c34)n2)cc1 ; c1ccc(-c2ccc3c(c2)c2ccccc2n3-c2ccc3oc4cccc(-c5nc(-c6ccccc6)nc(-c6ccc7c(c6)sc6cccc(-c8ccccc8)c67)n5)c4c3c2)cc1]

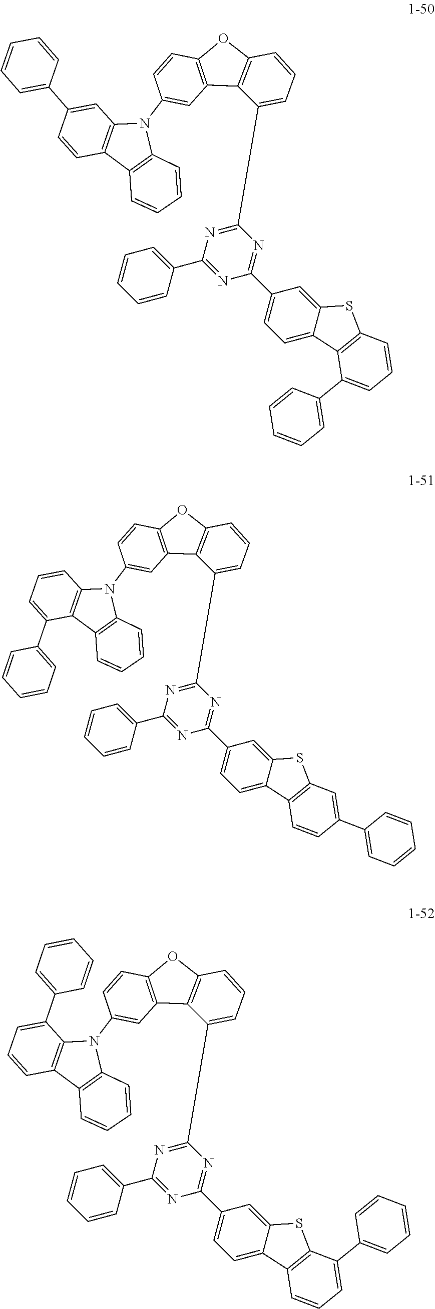 [SMILES: c1ccc(-c2ccc3c4ccccc4n(-c4ccc5oc6cccc(-c7nc(-c8ccccc8)nc(-c8ccc9c(c8)sc8cccc(-c%10ccccc%10)c89)n7)c6c5c4)c3c2)cc1 ; c1ccc(-c2ccc3c(c2)sc2cc(-c4nc(-c5ccccc5)nc(-c5cccc6oc7ccc(-n8c9ccccc9c9c(-c%10ccccc%10)cccc98)cc7c56)n4)ccc23)cc1 ; c1ccc(-c2nc(-c3ccc4c(c3)sc3c(-c5ccccc5)cccc34)nc(-c3cccc4oc5ccc(-n6c7ccccc7c7cccc(-c8ccccc8)c76)cc5c34)n2)cc1]

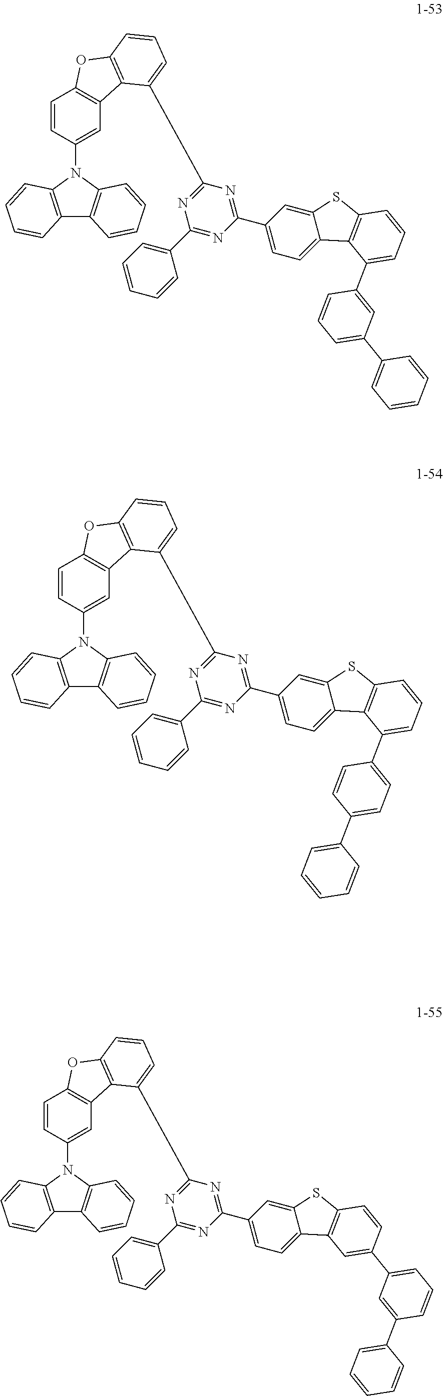 [SMILES: c1ccc(-c2cccc(-c3cccc4sc5cc(-c6nc(-c7ccccc7)nc(-c7cccc8oc9ccc(-n%10c%11ccccc%11c%11ccccc%11%10)cc9c78)n6)ccc5c34)c2)cc1 ; c1ccc(-c2ccc(-c3cccc4sc5cc(-c6nc(-c7ccccc7)nc(-c7cccc8oc9ccc(-n%10c%11ccccc%11c%11ccccc%11%10)cc9c78)n6)ccc5c34)cc2)cc1 ; c1ccc(-c2cccc(-c3ccc4sc5cc(-c6nc(-c7ccccc7)nc(-c7cccc8oc9ccc(-n%10c%11ccccc%11c%11ccccc%11%10)cc9c78)n6)ccc5c4c3)c2)cc1]

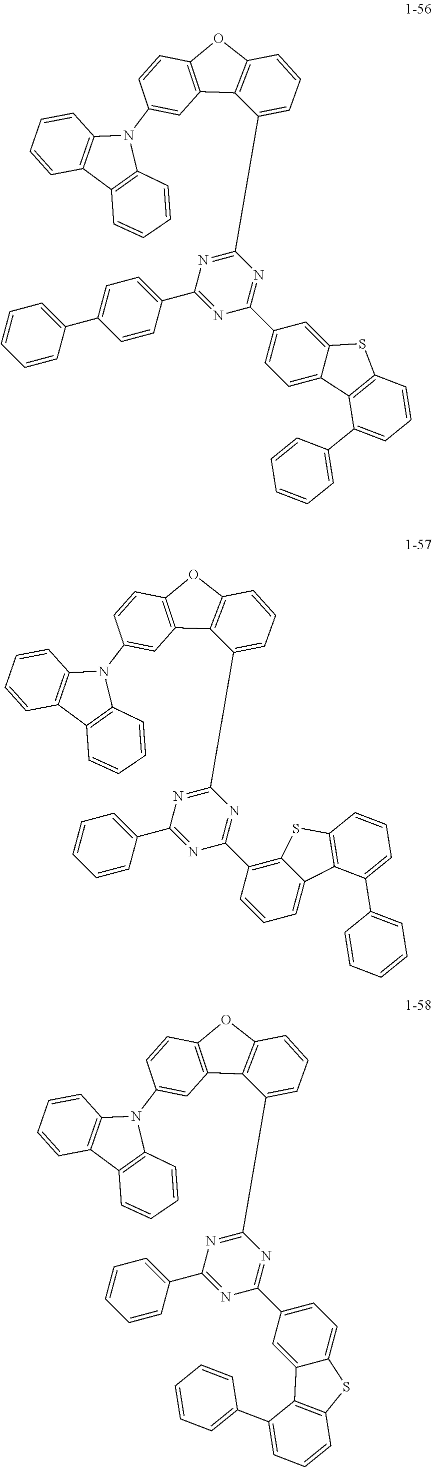 [SMILES: c1ccc(-c2ccc(-c3nc(-c4ccc5c(c4)sc4cccc(-c6ccccc6)c45)nc(-c4cccc5oc6ccc(-n7c8ccccc8c8ccccc87)cc6c45)n3)cc2)cc1 ; c1ccc(-c2nc(-c3cccc4c3sc3cccc(-c5ccccc5)c34)nc(-c3cccc4oc5ccc(-n6c7ccccc7c7ccccc76)cc5c34)n2)cc1 ; c1ccc(-c2nc(-c3ccc4sc5cccc(-c6ccccc6)c5c4c3)nc(-c3cccc4oc5ccc(-n6c7ccccc7c7ccccc76)cc5c34)n2)cc1]

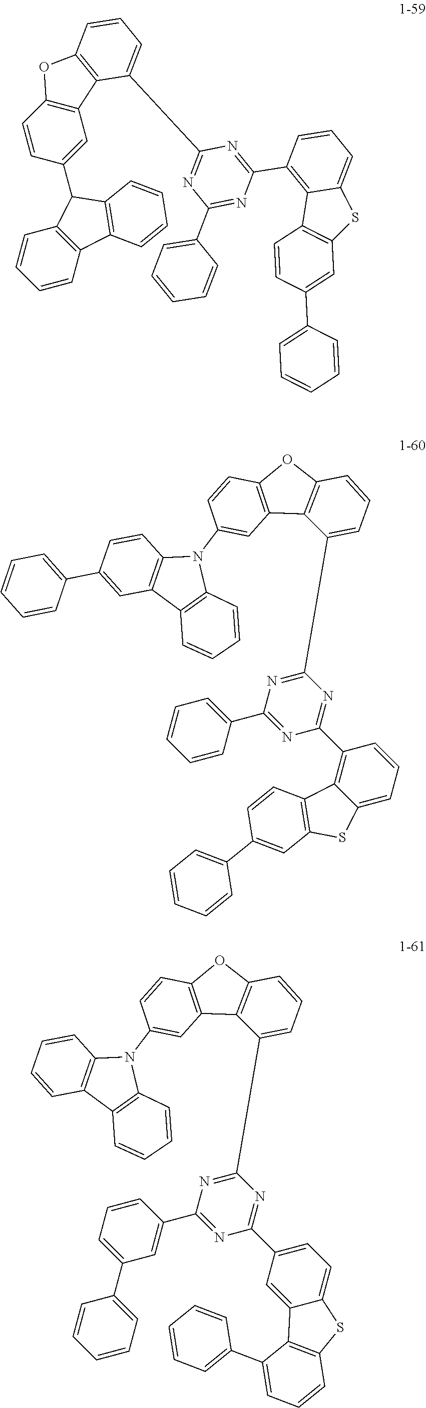

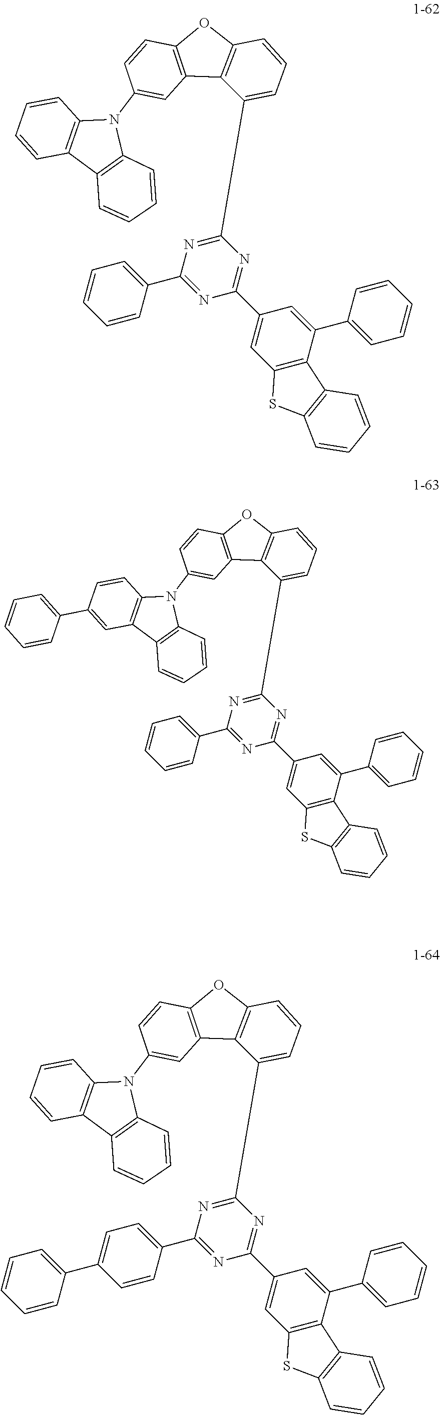 [SMILES: c1ccc(-c2nc(-c3cc(-c4ccccc4)c4c(c3)sc3ccccc34)nc(-c3cccc4oc5ccc(-n6c7ccccc7c7ccccc76)cc5c34)n2)cc1 ; c1ccc(-c2ccc3c(c2)c2ccccc2n3-c2ccc3oc4cccc(-c5nc(-c6ccccc6)nc(-c6cc(-c7ccccc7)c7c(c6)sc6ccccc67)n5)c4c3c2)cc1 ; c1ccc(-c2ccc(-c3nc(-c4cc(-c5ccccc5)c5c(c4)sc4ccccc45)nc(-c4cccc5oc6ccc(-n7c8ccccc8c8ccccc87)cc6c45)n3)cc2)cc1]

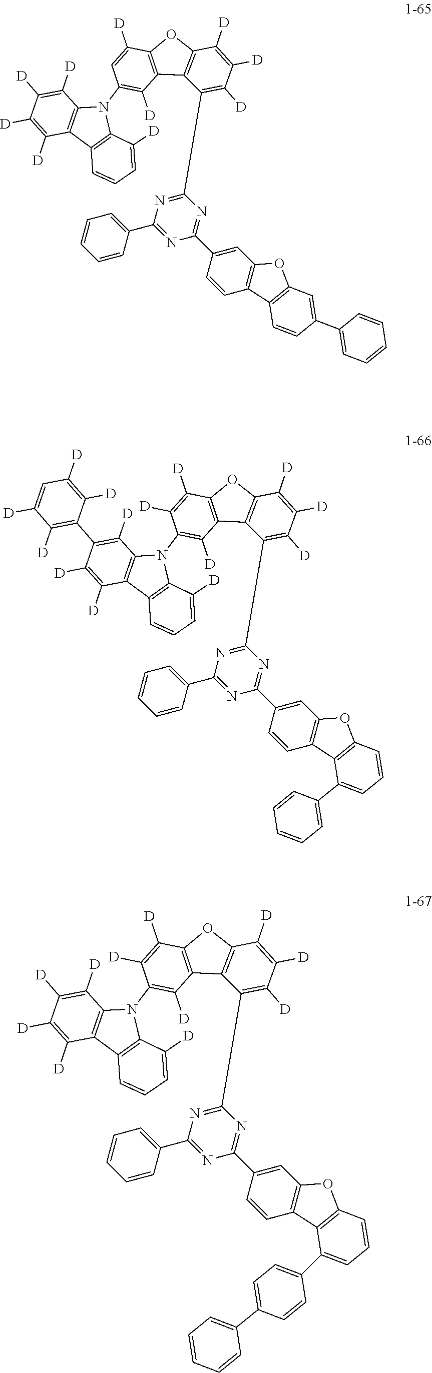

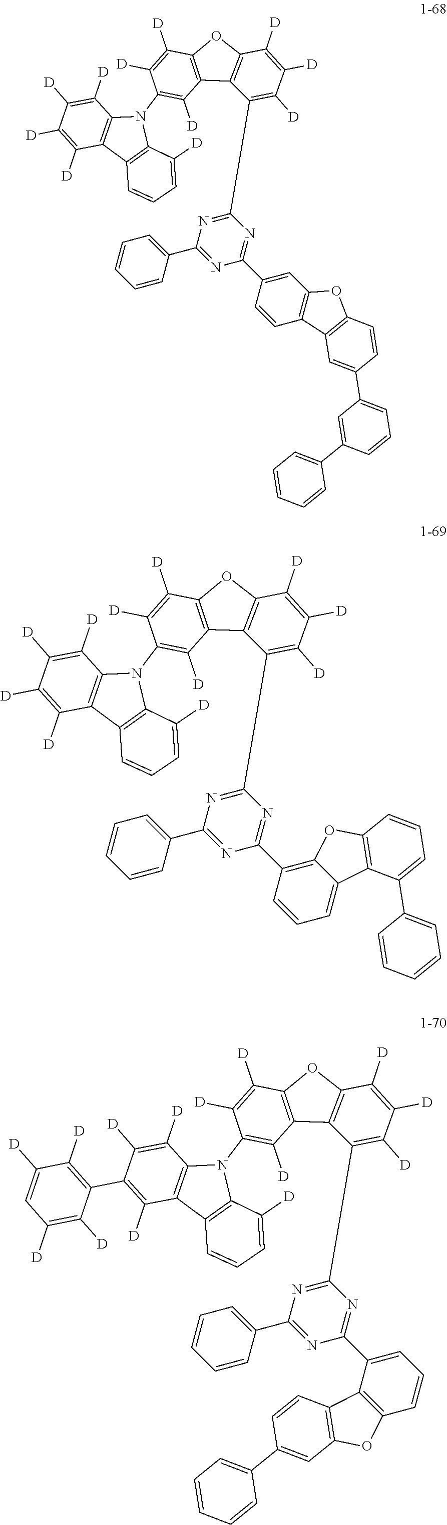

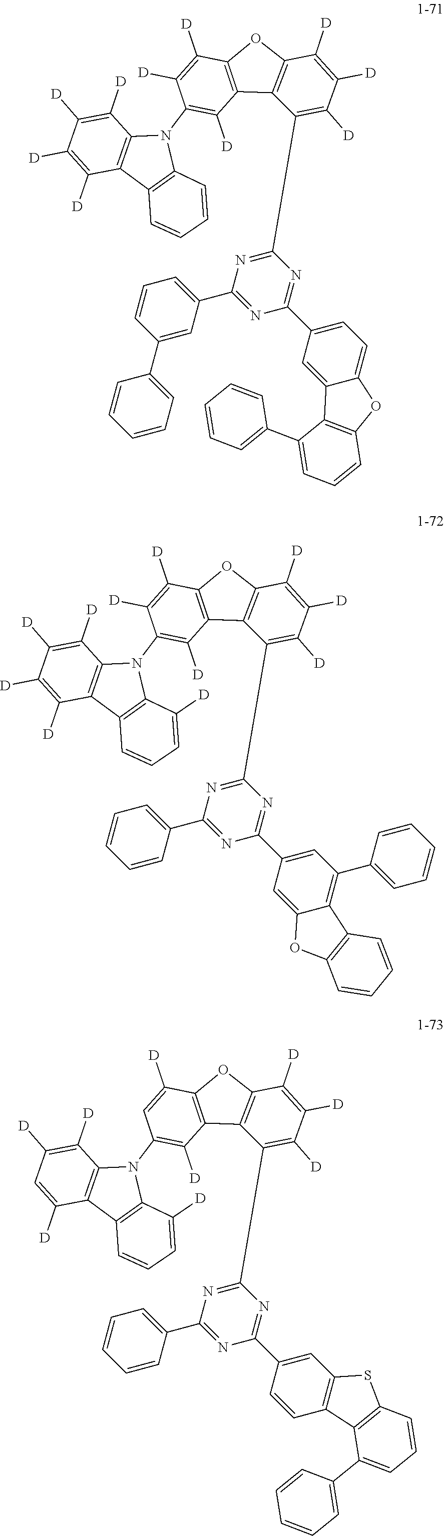 [SMILES: [2H]c1c(-n2c3ccccc3c3c([2H])c([2H])c([2H])c([2H])c32)c([2H])c2c(oc3c([2H])c([2H])c([2H])c(-c4nc(-c5cccc(-c6ccccc6)c5)nc(-c5ccc6oc7cccc(-c8ccccc8)c7c6c5)n4)c32)c1[2H] ; [2H]c1c(-n2c3c([2H])cccc3c3c([2H])c([2H])c([2H])c([2H])c32)c([2H])c2c(oc3c([2H])c([2H])c([2H])c(-c4nc(-c5ccccc5)nc(-c5cc(-c6ccccc6)c6c(c5)oc5ccccc56)n4)c32)c1[2H] ; [2H]c1cc([2H])c2c3cccc([2H])c3n(-c3cc([2H])c4oc5c([2H])c([2H])c([2H])c(-c6nc(-c7ccccc7)nc(-c7ccc8c(c7)sc7cccc(-c9ccccc9)c78)n6)c5c4c3[2H])c2c1[2H]]

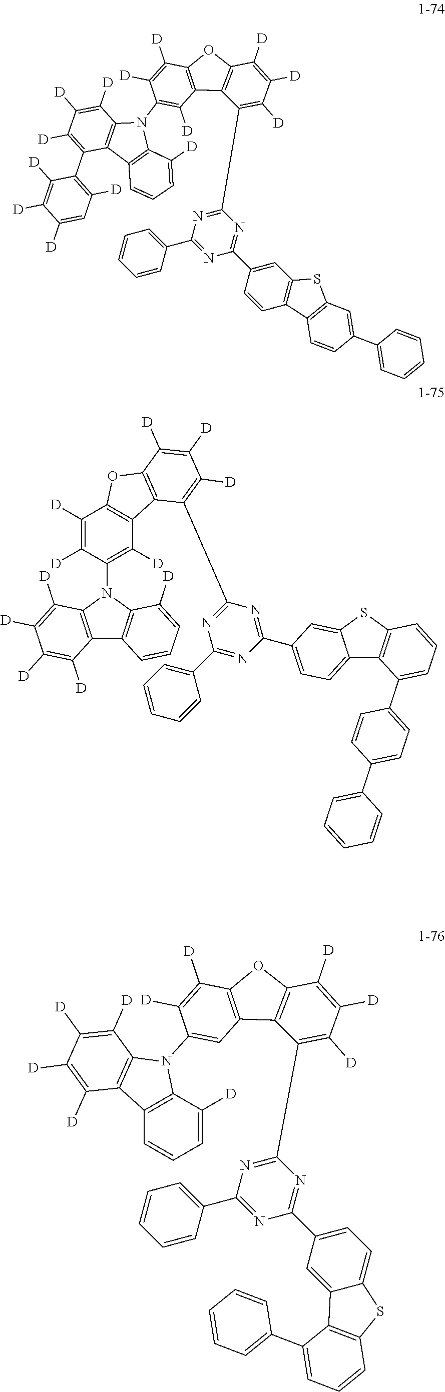

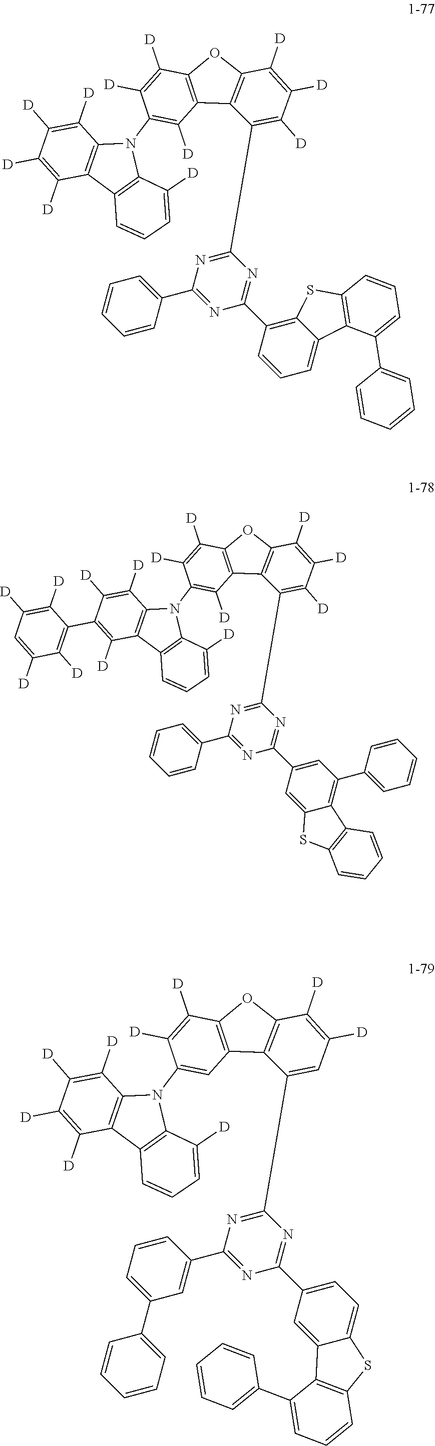

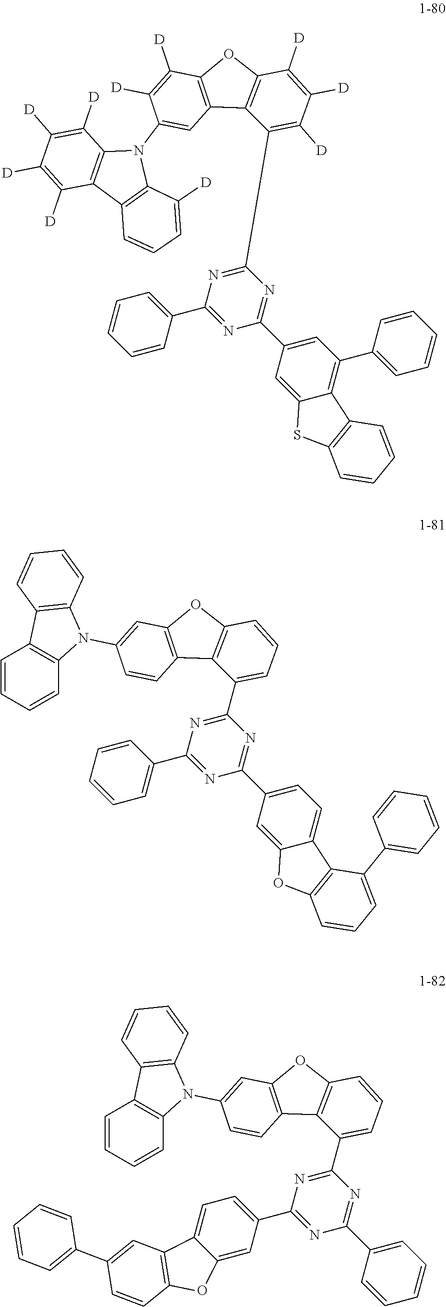 [SMILES: [2H]c1c(-n2c3c([2H])cccc3c3c([2H])c([2H])c([2H])c([2H])c32)cc2c(oc3c([2H])c([2H])c([2H])c(-c4nc(-c5ccccc5)nc(-c5cc(-c6ccccc6)c6c(c5)sc5ccccc56)n4)c32)c1[2H] ; c1ccc(-c2nc(-c3ccc4c(c3)oc3cccc(-c5ccccc5)c34)nc(-c3cccc4oc5cc(-n6c7ccccc7c7ccccc76)ccc5c34)n2)cc1 ; c1ccc(-c2ccc3oc4cc(-c5nc(-c6ccccc6)nc(-c6cccc7oc8cc(-n9c%10ccccc%10c%10ccccc%109)ccc8c67)n5)ccc4c3c2)cc1]

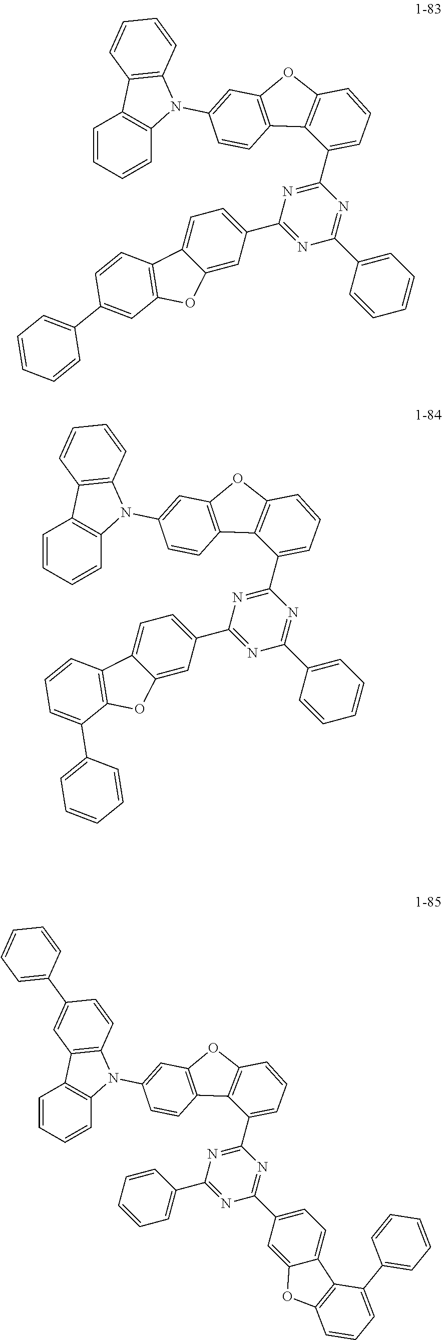 [SMILES: c1ccc(-c2ccc3c(c2)oc2cc(-c4nc(-c5ccccc5)nc(-c5cccc6oc7cc(-n8c9ccccc9c9ccccc98)ccc7c56)n4)ccc23)cc1 ; c1ccc(-c2nc(-c3ccc4c(c3)oc3c(-c5ccccc5)cccc34)nc(-c3cccc4oc5cc(-n6c7ccccc7c7ccccc76)ccc5c34)n2)cc1 ; c1ccc(-c2ccc3c(c2)c2ccccc2n3-c2ccc3c(c2)oc2cccc(-c4nc(-c5ccccc5)nc(-c5ccc6c(c5)oc5cccc(-c7ccccc7)c56)n4)c23)cc1]

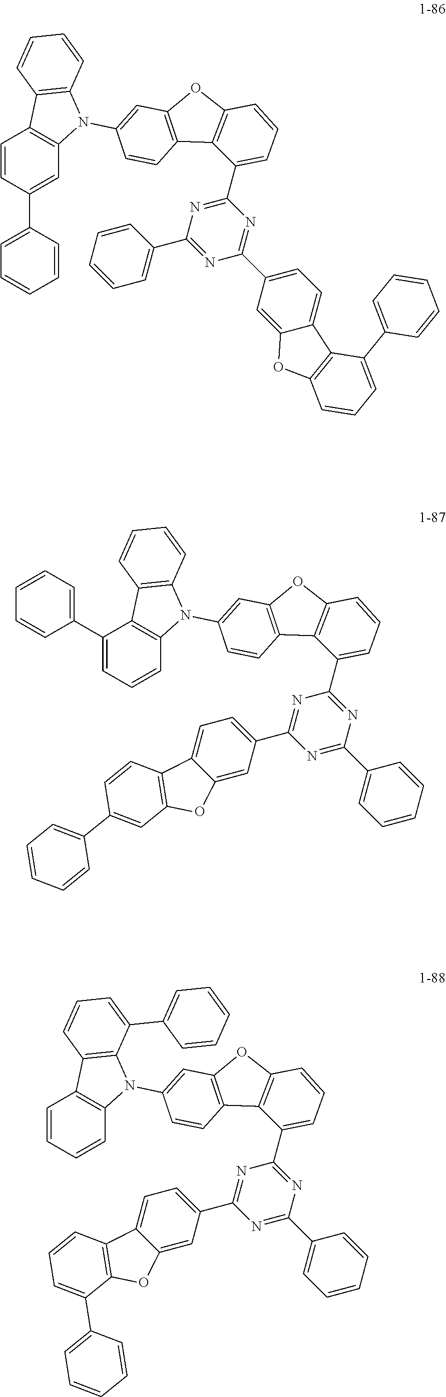 [SMILES: c1ccc(-c2ccc3c4ccccc4n(-c4ccc5c(c4)oc4cccc(-c6nc(-c7ccccc7)nc(-c7ccc8c(c7)oc7cccc(-c9ccccc9)c78)n6)c45)c3c2)cc1 ; c1ccc(-c2ccc3c(c2)oc2cc(-c4nc(-c5ccccc5)nc(-c5cccc6oc7cc(-n8c9ccccc9c9c(-c%10ccccc%10)cccc98)ccc7c56)n4)ccc23)cc1 ; c1ccc(-c2nc(-c3ccc4c(c3)oc3c(-c5ccccc5)cccc34)nc(-c3cccc4oc5cc(-n6c7ccccc7c7cccc(-c8ccccc8)c76)ccc5c34)n2)cc1]

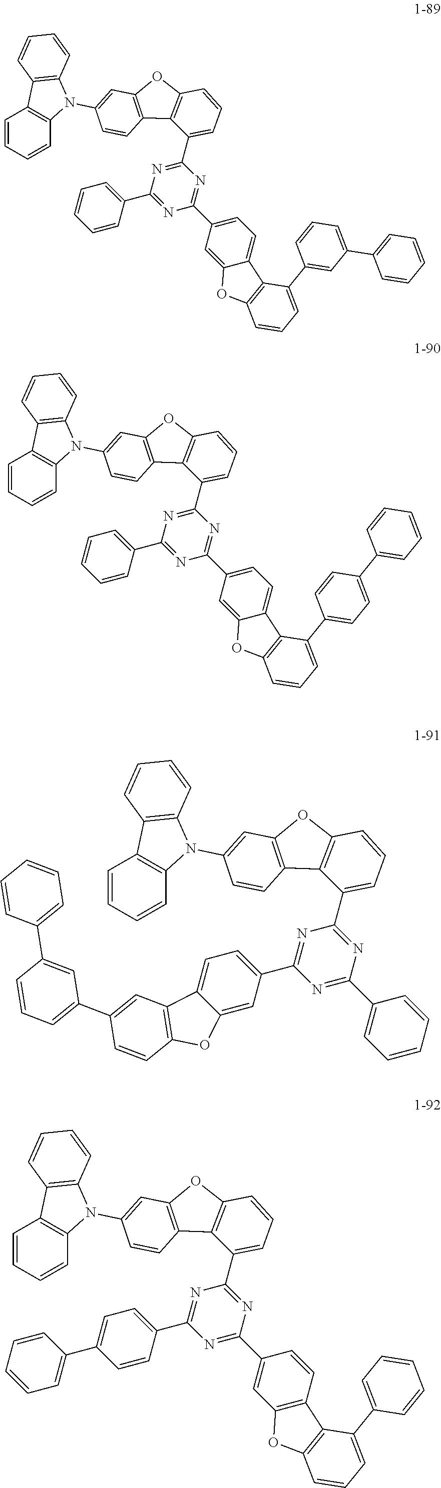 [SMILES: c1ccc(-c2cccc(-c3cccc4oc5cc(-c6nc(-c7ccccc7)nc(-c7cccc8oc9cc(-n%10c%11ccccc%11c%11ccccc%11%10)ccc9c78)n6)ccc5c34)c2)cc1 ; c1ccc(-c2ccc(-c3cccc4oc5cc(-c6nc(-c7ccccc7)nc(-c7cccc8oc9cc(-n%10c%11ccccc%11c%11ccccc%11%10)ccc9c78)n6)ccc5c34)cc2)cc1 ; c1ccc(-c2cccc(-c3ccc4oc5cc(-c6nc(-c7ccccc7)nc(-c7cccc8oc9cc(-n%10c%11ccccc%11c%11ccccc%11%10)ccc9c78)n6)ccc5c4c3)c2)cc1 ; c1ccc(-c2ccc(-c3nc(-c4ccc5c(c4)oc4cccc(-c6ccccc6)c45)nc(-c4cccc5oc6cc(-n7c8ccccc8c8ccccc87)ccc6c45)n3)cc2)cc1]

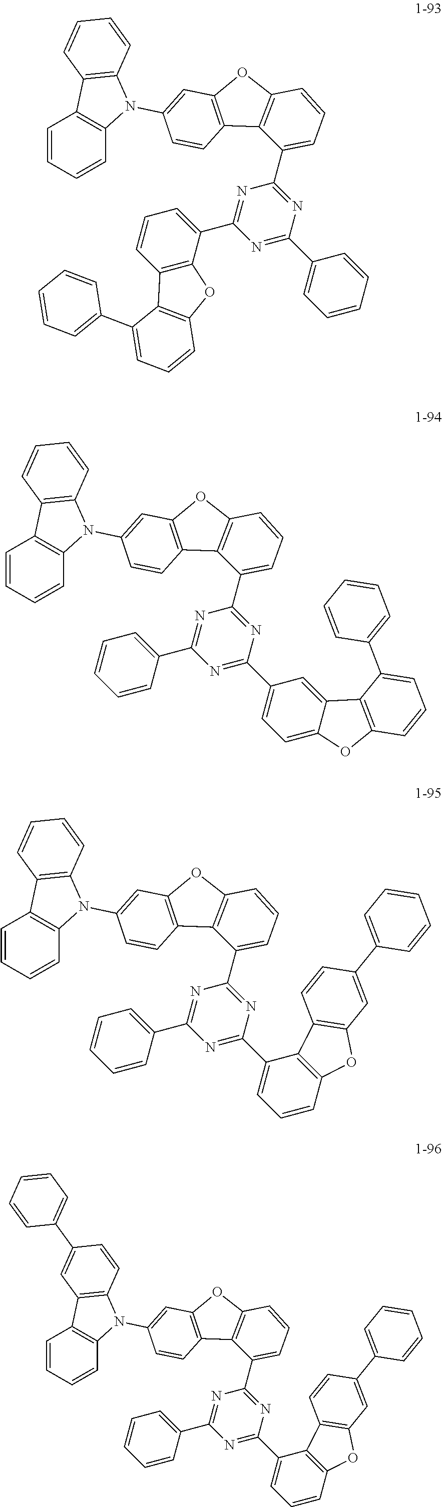 [SMILES: c1ccc(-c2nc(-c3cccc4c3oc3cccc(-c5ccccc5)c34)nc(-c3cccc4oc5cc(-n6c7ccccc7c7ccccc76)ccc5c34)n2)cc1 ; c1ccc(-c2nc(-c3ccc4oc5cccc(-c6ccccc6)c5c4c3)nc(-c3cccc4oc5cc(-n6c7ccccc7c7ccccc76)ccc5c34)n2)cc1 ; c1ccc(-c2ccc3c(c2)oc2cccc(-c4nc(-c5ccccc5)nc(-c5cccc6oc7cc(-n8c9ccccc9c9ccccc98)ccc7c56)n4)c23)cc1 ; c1ccc(-c2ccc3c(c2)oc2cccc(-c4nc(-c5ccccc5)nc(-c5cccc6oc7cc(-n8c9ccccc9c9cc(-c%10ccccc%10)ccc98)ccc7c56)n4)c23)cc1]

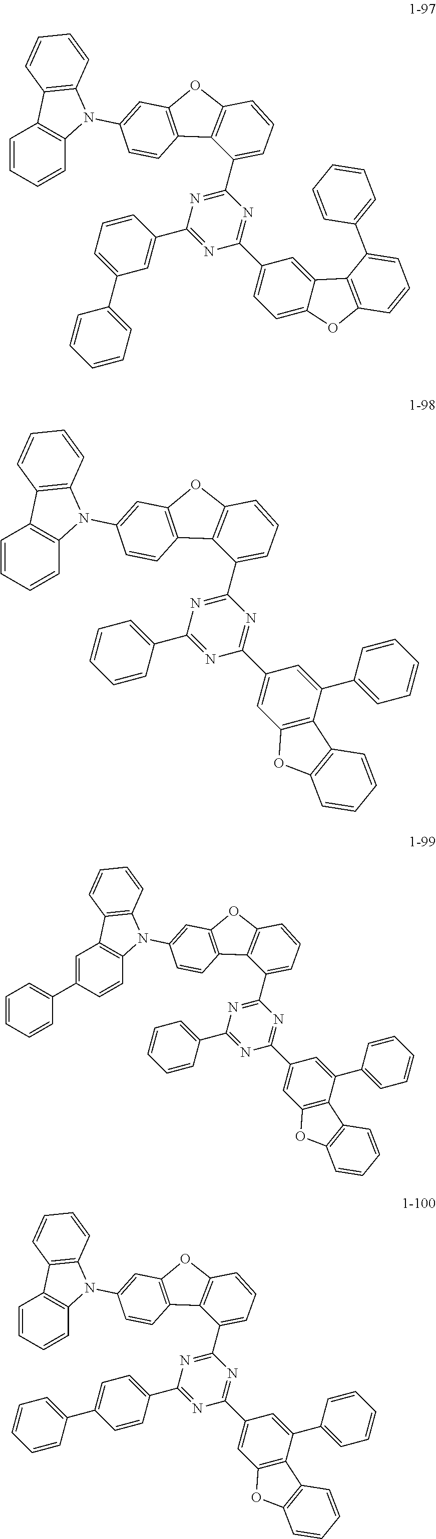 [SMILES: c1ccc(-c2cccc(-c3nc(-c4ccc5oc6cccc(-c7ccccc7)c6c5c4)nc(-c4cccc5oc6cc(-n7c8ccccc8c8ccccc87)ccc6c45)n3)c2)cc1 ; c1ccc(-c2nc(-c3cc(-c4ccccc4)c4c(c3)oc3ccccc34)nc(-c3cccc4oc5cc(-n6c7ccccc7c7ccccc76)ccc5c34)n2)cc1 ; c1ccc(-c2ccc3c(c2)c2ccccc2n3-c2ccc3c(c2)oc2cccc(-c4nc(-c5ccccc5)nc(-c5cc(-c6ccccc6)c6c(c5)oc5ccccc56)n4)c23)cc1 ; c1ccc(-c2ccc(-c3nc(-c4cc(-c5ccccc5)c5c(c4)oc4ccccc45)nc(-c4cccc5oc6cc(-n7c8ccccc8c8ccccc87)ccc6c45)n3)cc2)cc1]

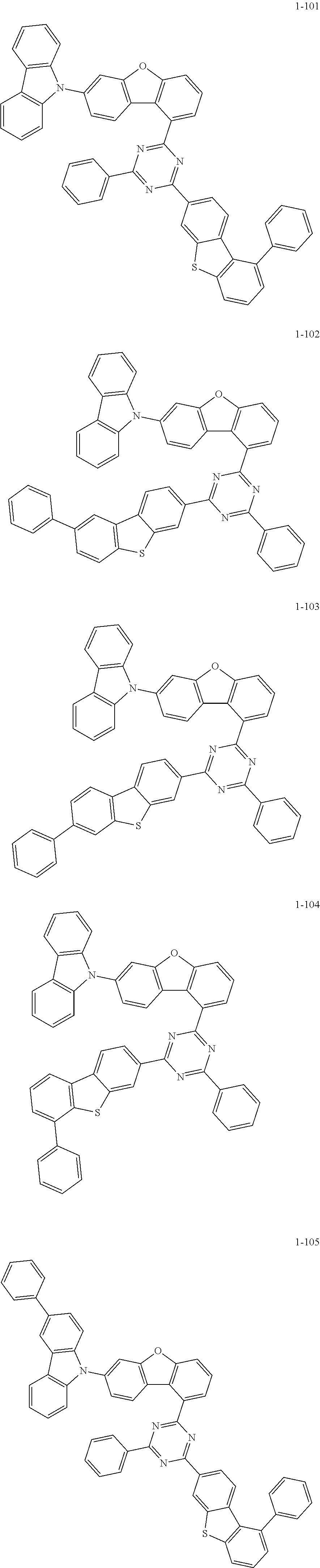

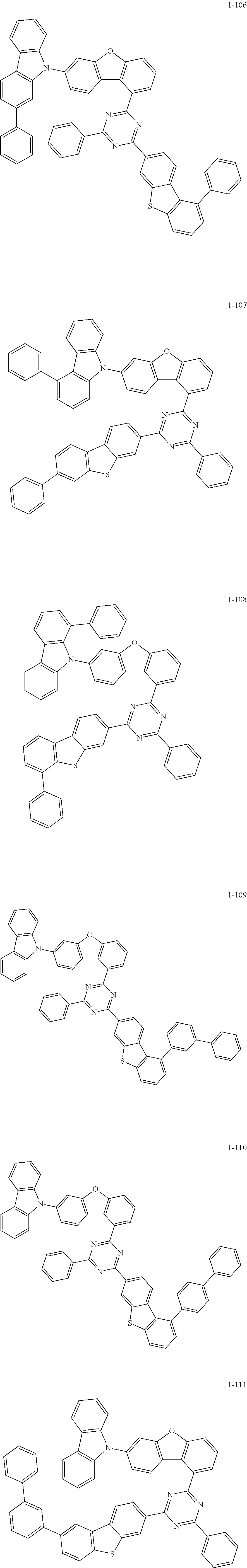

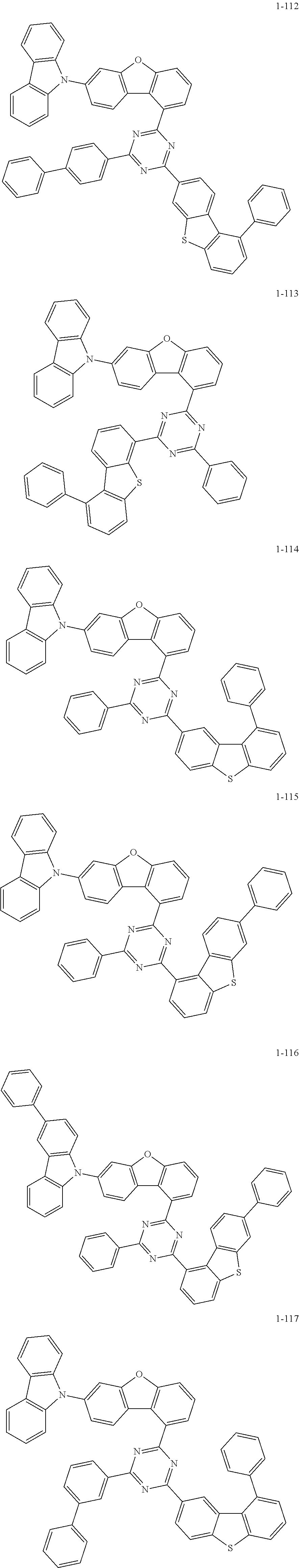

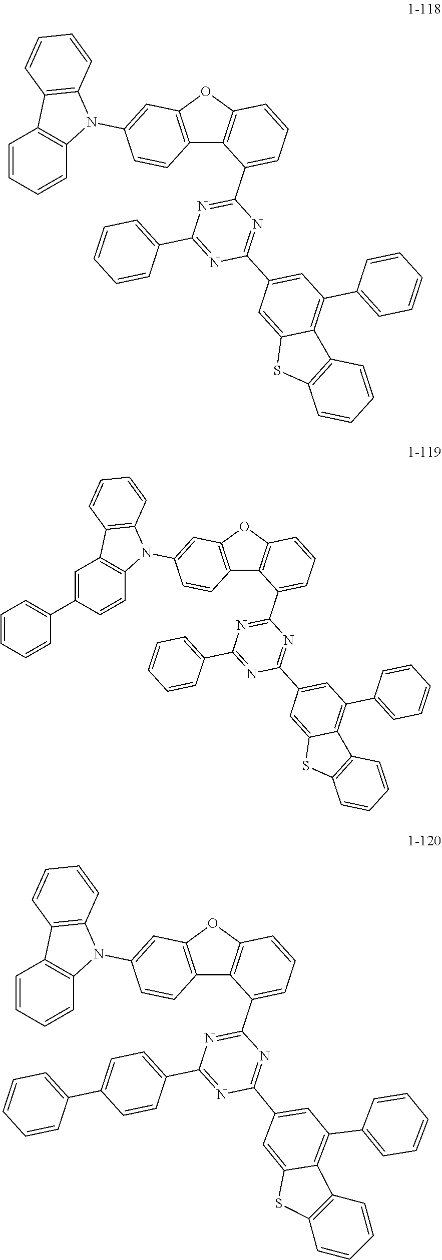 [SMILES: c1ccc(-c2nc(-c3cc(-c4ccccc4)c4c(c3)sc3ccccc34)nc(-c3cccc4oc5cc(-n6c7ccccc7c7ccccc76)ccc5c34)n2)cc1 ; c1ccc(-c2ccc3c(c2)c2ccccc2n3-c2ccc3c(c2)oc2cccc(-c4nc(-c5ccccc5)nc(-c5cc(-c6ccccc6)c6c(c5)sc5ccccc56)n4)c23)cc1 ; c1ccc(-c2ccc(-c3nc(-c4cc(-c5ccccc5)c5c(c4)sc4ccccc45)nc(-c4cccc5oc6cc(-n7c8ccccc8c8ccccc87)ccc6c45)n3)cc2)cc1]

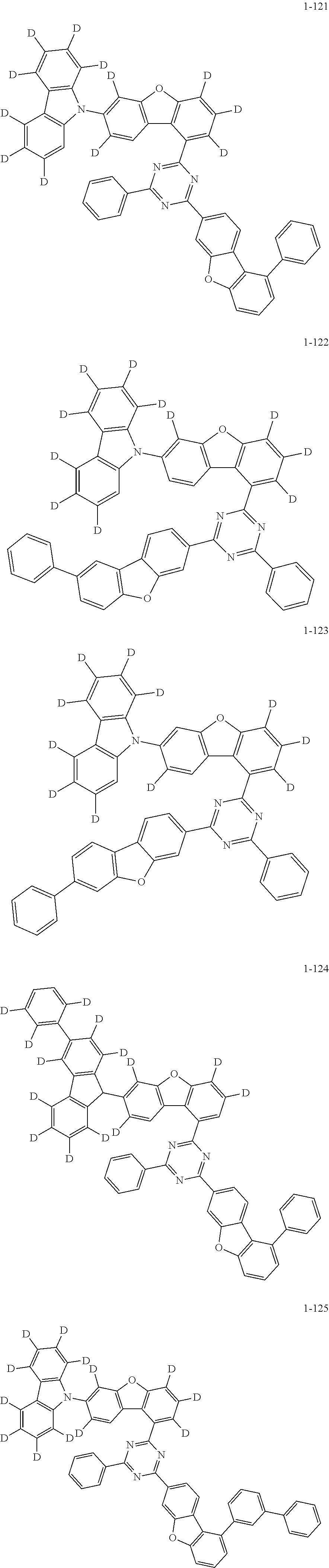

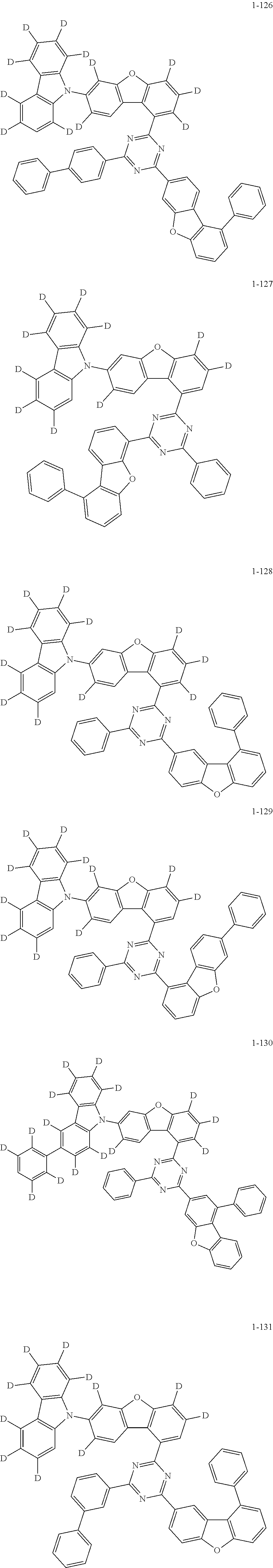

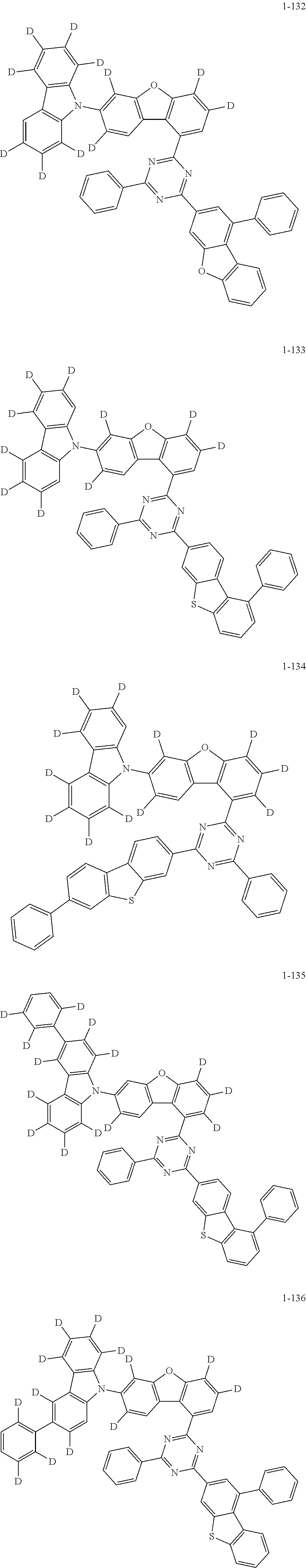

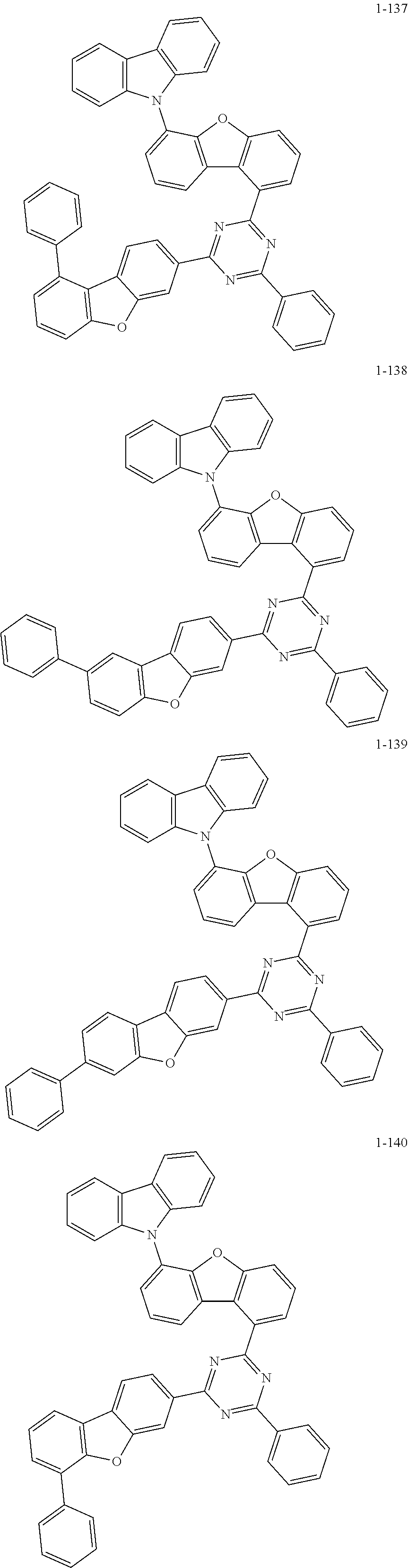 [SMILES: c1ccc(-c2nc(-c3ccc4c(c3)oc3cccc(-c5ccccc5)c34)nc(-c3cccc4oc5c(-n6c7ccccc7c7ccccc76)cccc5c34)n2)cc1 ; c1ccc(-c2ccc3oc4cc(-c5nc(-c6ccccc6)nc(-c6cccc7oc8c(-n9c%10ccccc%10c%10ccccc%109)cccc8c67)n5)ccc4c3c2)cc1 ; c1ccc(-c2ccc3c(c2)oc2cc(-c4nc(-c5ccccc5)nc(-c5cccc6oc7c(-n8c9ccccc9c9ccccc98)cccc7c56)n4)ccc23)cc1 ; c1ccc(-c2nc(-c3ccc4c(c3)oc3c(-c5ccccc5)cccc34)nc(-c3cccc4oc5c(-n6c7ccccc7c7ccccc76)cccc5c34)n2)cc1]

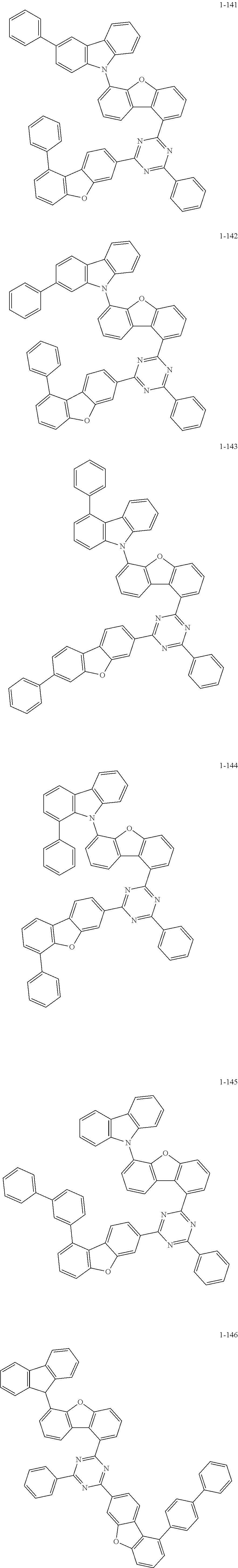

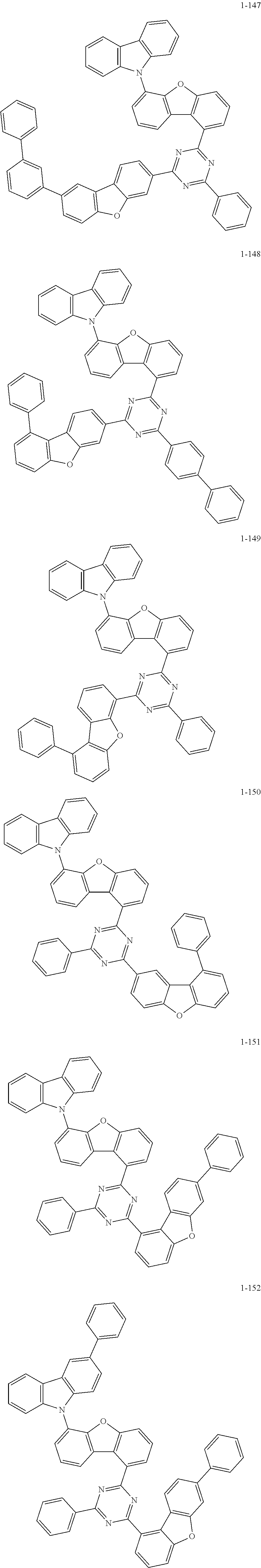

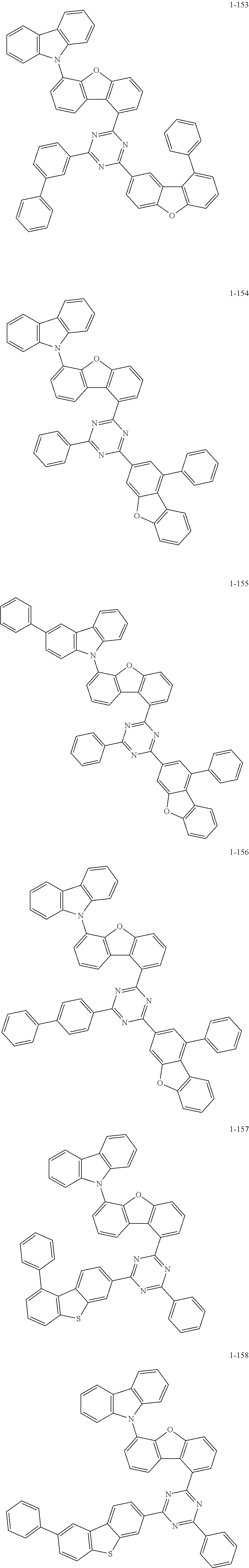

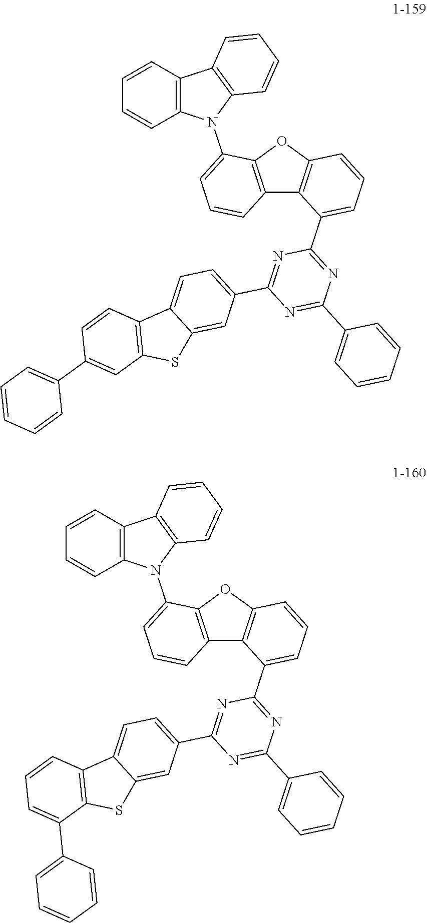 [SMILES: c1ccc(-c2ccc3c(c2)sc2cc(-c4nc(-c5ccccc5)nc(-c5cccc6oc7c(-n8c9ccccc9c9ccccc98)cccc7c56)n4)ccc23)cc1 ; c1ccc(-c2nc(-c3ccc4c(c3)sc3c(-c5ccccc5)cccc34)nc(-c3cccc4oc5c(-n6c7ccccc7c7ccccc76)cccc5c34)n2)cc1]

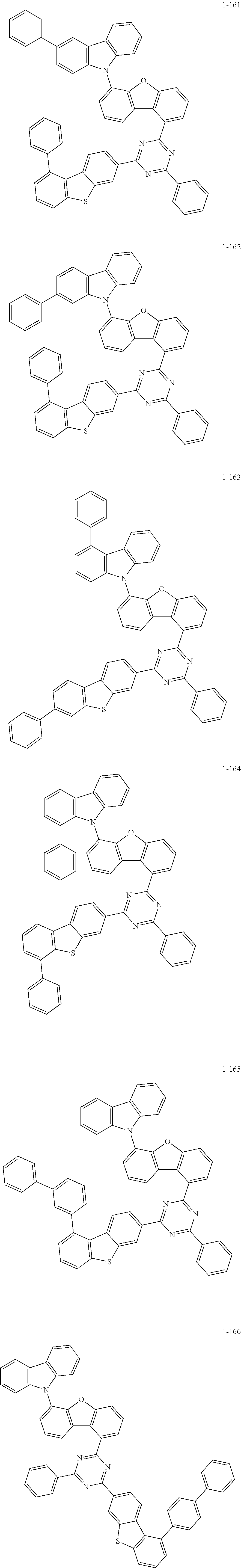

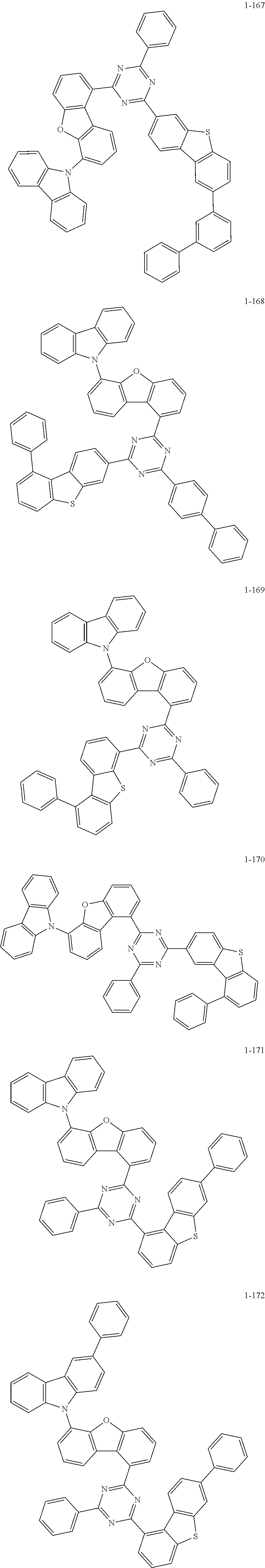

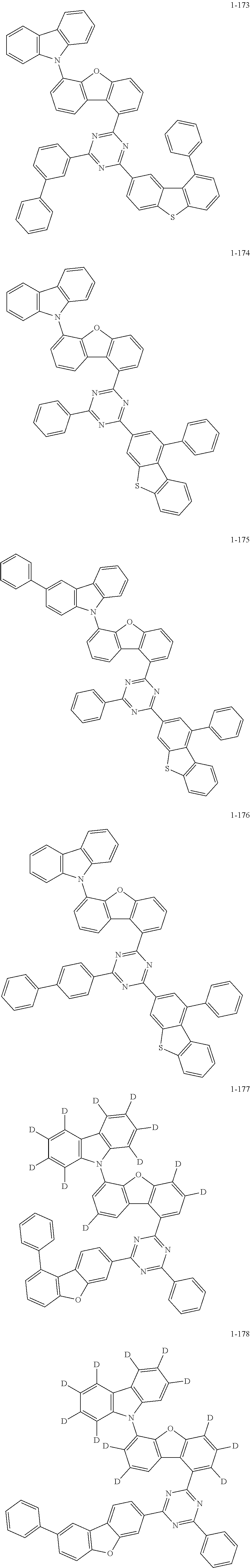

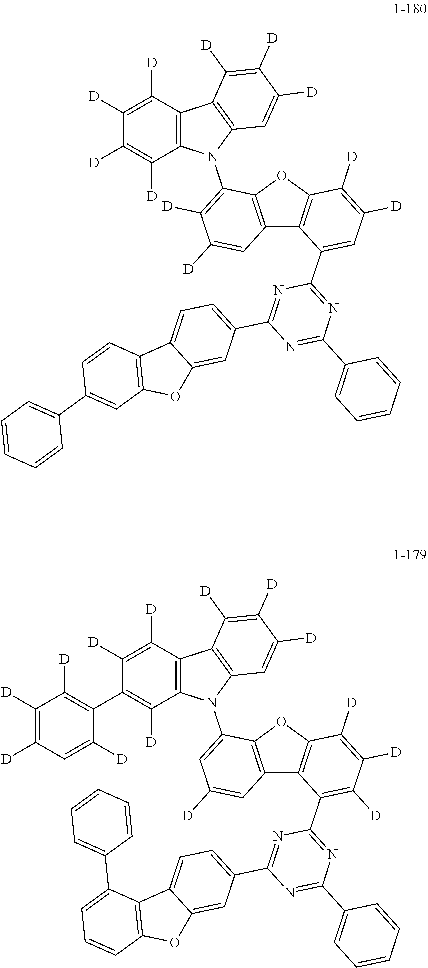 [SMILES: [2H]c1cc2c(oc3c([2H])c([2H])cc(-c4nc(-c5ccccc5)nc(-c5ccc6c(c5)oc5cc(-c7ccccc7)ccc56)n4)c32)c(-n2c3cc([2H])c([2H])c([2H])c3c3c([2H])c([2H])c([2H])c([2H])c32)c1[2H] ; [2H]c1cc(-n2c3cc([2H])c([2H])c([2H])c3c3c([2H])c([2H])c(-c4c([2H])cc([2H])c([2H])c4[2H])c([2H])c32)c2oc3c([2H])c([2H])c([2H])c(-c4nc(-c5ccccc5)nc(-c5ccc6c(c5)oc5cccc(-c7ccccc7)c56)n4)c3c2c1]

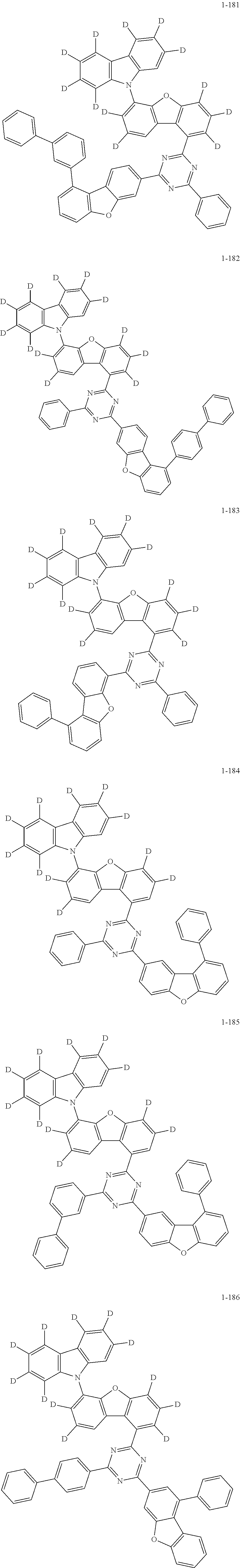

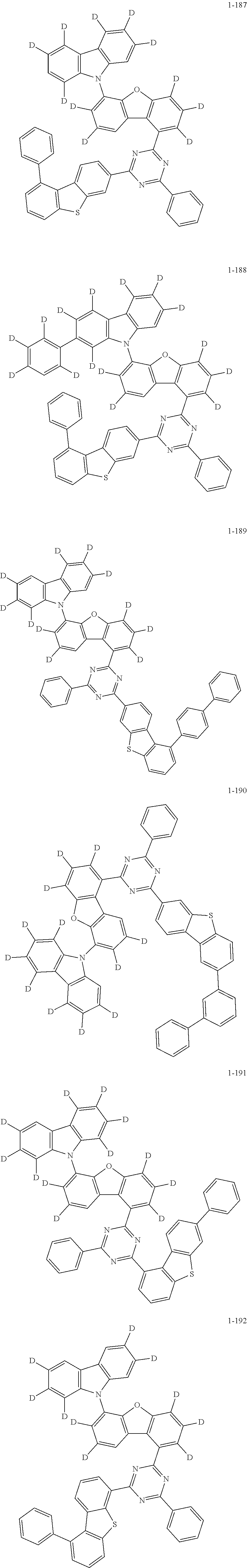

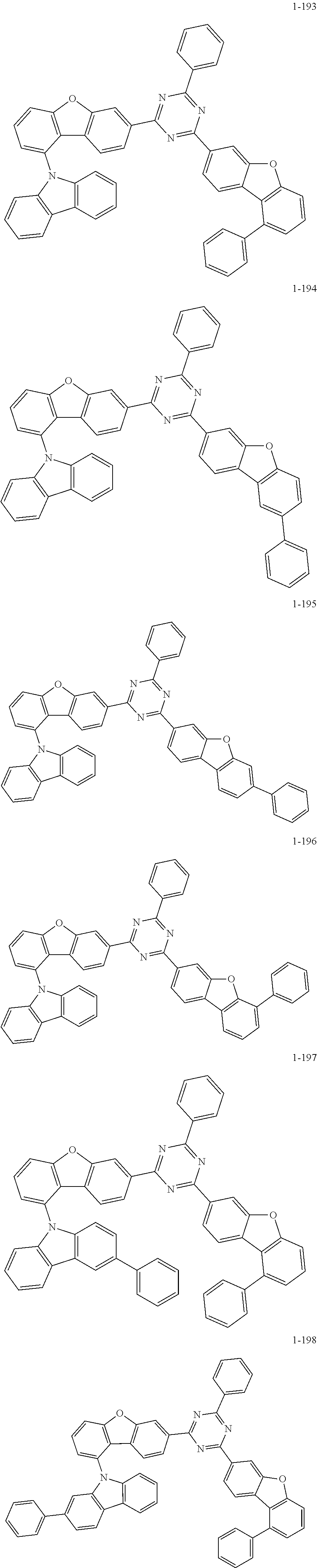

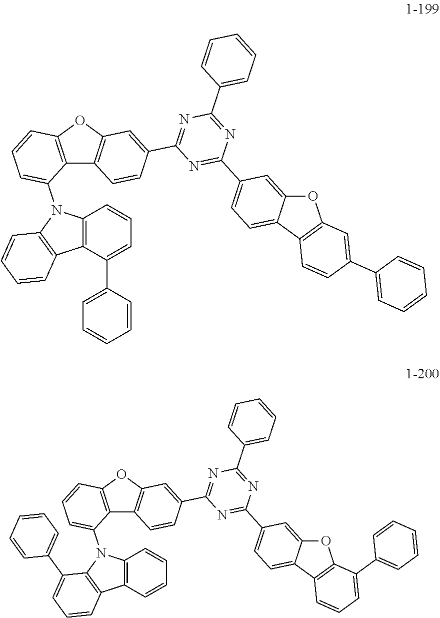 [SMILES: c1ccc(-c2ccc3c(c2)oc2cc(-c4nc(-c5ccccc5)nc(-c5ccc6c(c5)oc5cccc(-n7c8ccccc8c8c(-c9ccccc9)cccc87)c56)n4)ccc23)cc1 ; c1ccc(-c2nc(-c3ccc4c(c3)oc3c(-c5ccccc5)cccc34)nc(-c3ccc4c(c3)oc3cccc(-n5c6ccccc6c6cccc(-c7ccccc7)c65)c34)n2)cc1]

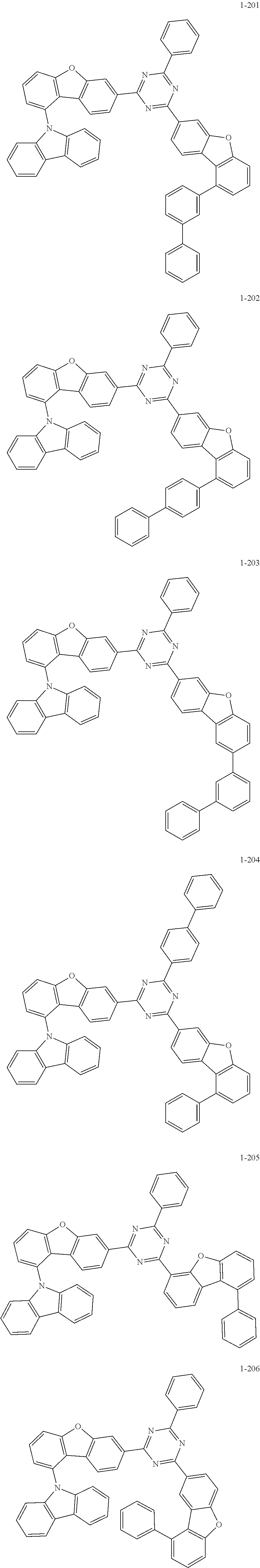

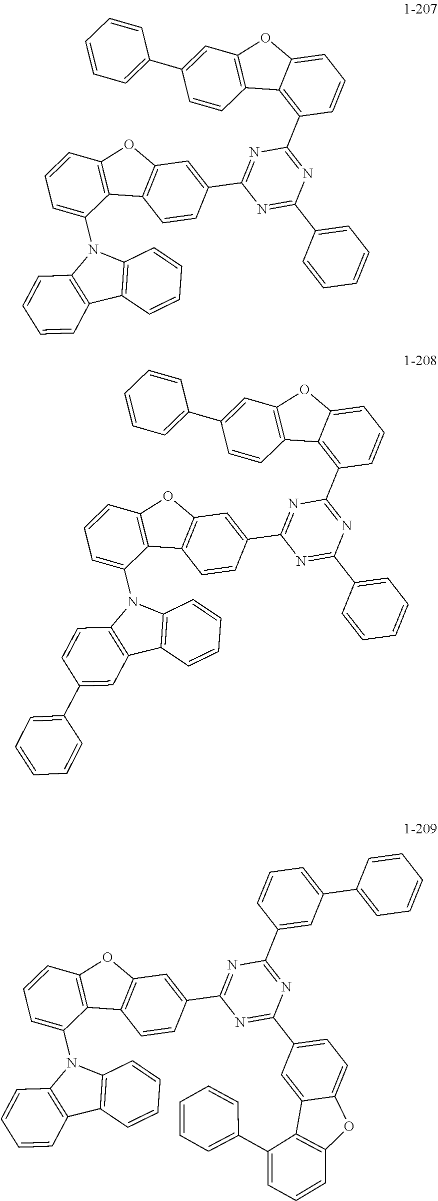 [SMILES: c1ccc(-c2ccc3c(c2)oc2cccc(-c4nc(-c5ccccc5)nc(-c5ccc6c(c5)oc5cccc(-n7c8ccccc8c8ccccc87)c56)n4)c23)cc1 ; c1ccc(-c2ccc3c(c2)oc2cccc(-c4nc(-c5ccccc5)nc(-c5ccc6c(c5)oc5cccc(-n7c8ccccc8c8cc(-c9ccccc9)ccc87)c56)n4)c23)cc1 ; c1ccc(-c2cccc(-c3nc(-c4ccc5c(c4)oc4cccc(-n6c7ccccc7c7ccccc76)c45)nc(-c4ccc5oc6cccc(-c7ccccc7)c6c5c4)n3)c2)cc1]

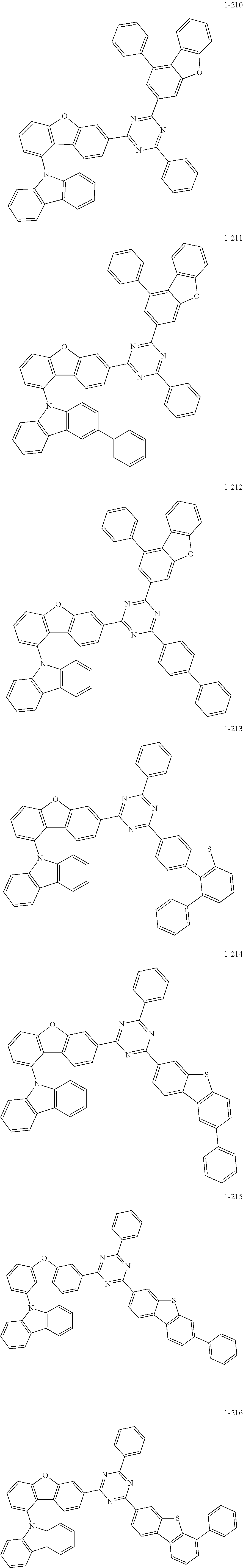

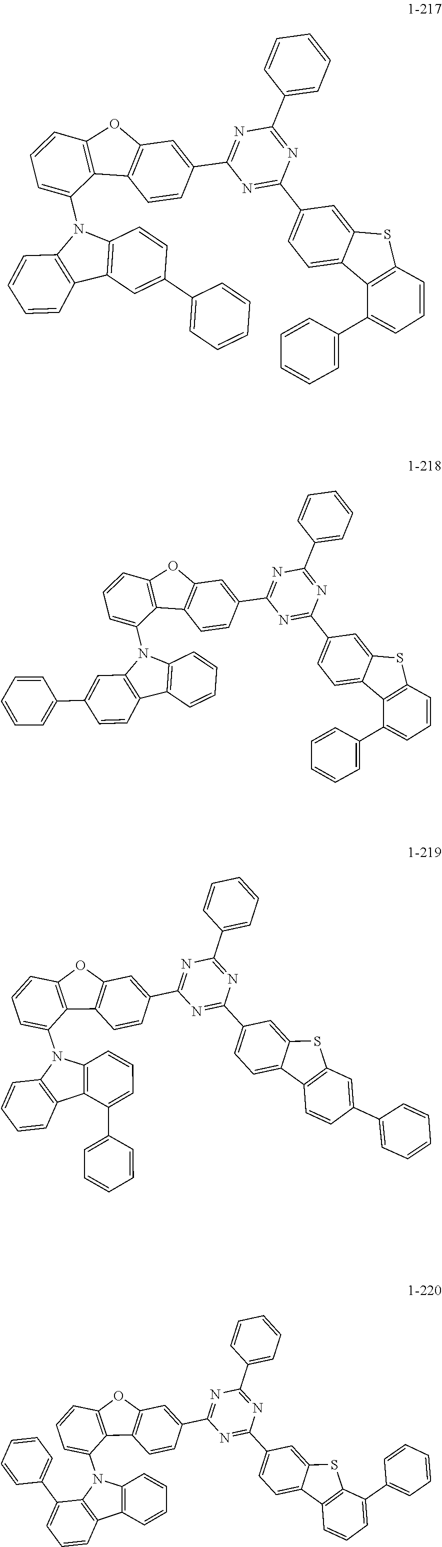 [SMILES: c1ccc(-c2ccc3c(c2)c2ccccc2n3-c2cccc3oc4cc(-c5nc(-c6ccccc6)nc(-c6ccc7c(c6)sc6cccc(-c8ccccc8)c67)n5)ccc4c23)cc1 ; c1ccc(-c2ccc3c4ccccc4n(-c4cccc5oc6cc(-c7nc(-c8ccccc8)nc(-c8ccc9c(c8)sc8cccc(-c%10ccccc%10)c89)n7)ccc6c45)c3c2)cc1 ; c1ccc(-c2ccc3c(c2)sc2cc(-c4nc(-c5ccccc5)nc(-c5ccc6c(c5)oc5cccc(-n7c8ccccc8c8c(-c9ccccc9)cccc87)c56)n4)ccc23)cc1 ; c1ccc(-c2nc(-c3ccc4c(c3)oc3cccc(-n5c6ccccc6c6cccc(-c7ccccc7)c65)c34)nc(-c3ccc4c(c3)sc3c(-c5ccccc5)cccc34)n2)cc1]

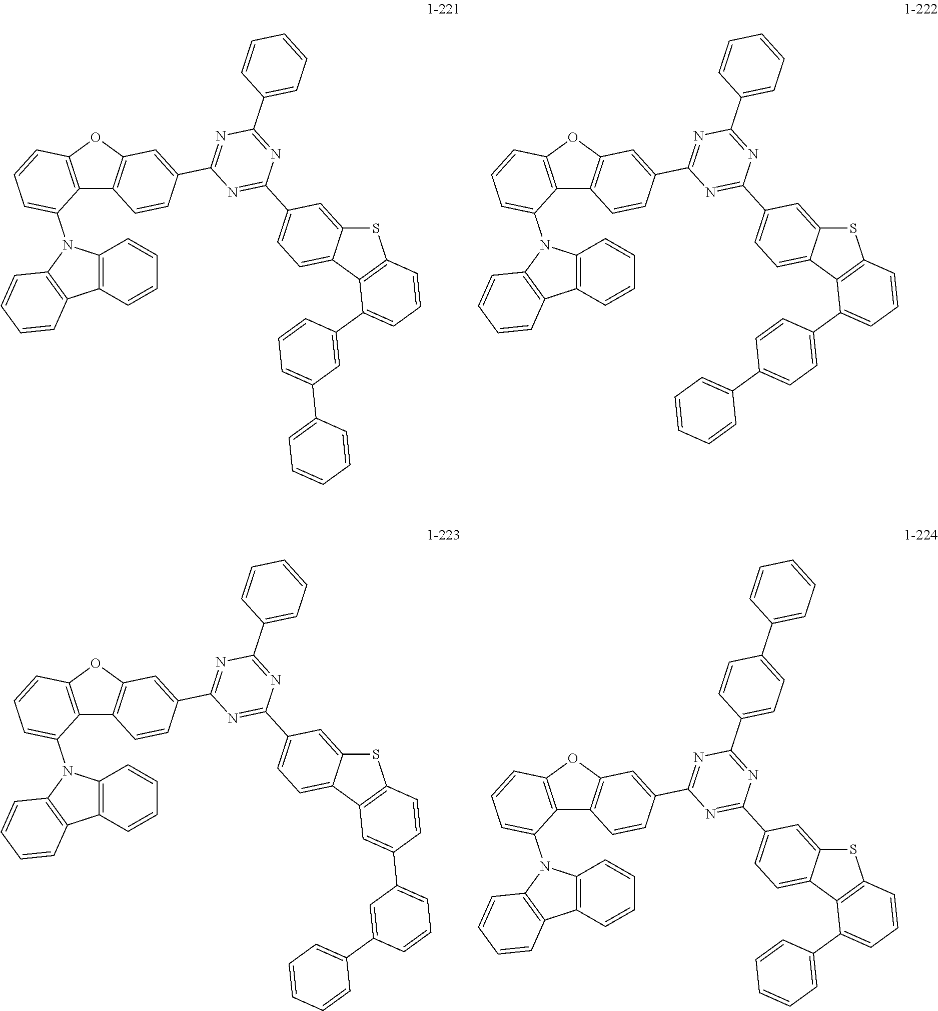 [SMILES: c1ccc(-c2cccc(-c3cccc4sc5cc(-c6nc(-c7ccccc7)nc(-c7ccc8c(c7)oc7cccc(-n9c%10ccccc%10c%10ccccc%109)c78)n6)ccc5c34)c2)cc1 ; c1ccc(-c2ccc(-c3cccc4sc5cc(-c6nc(-c7ccccc7)nc(-c7ccc8c(c7)oc7cccc(-n9c%10ccccc%10c%10ccccc%109)c78)n6)ccc5c34)cc2)cc1 ; c1ccc(-c2cccc(-c3ccc4sc5cc(-c6nc(-c7ccccc7)nc(-c7ccc8c(c7)oc7cccc(-n9c%10ccccc%10c%10ccccc%109)c78)n6)ccc5c4c3)c2)cc1 ; c1ccc(-c2ccc(-c3nc(-c4ccc5c(c4)oc4cccc(-n6c7ccccc7c7ccccc76)c45)nc(-c4ccc5c(c4)sc4cccc(-c6ccccc6)c45)n3)cc2)cc1]

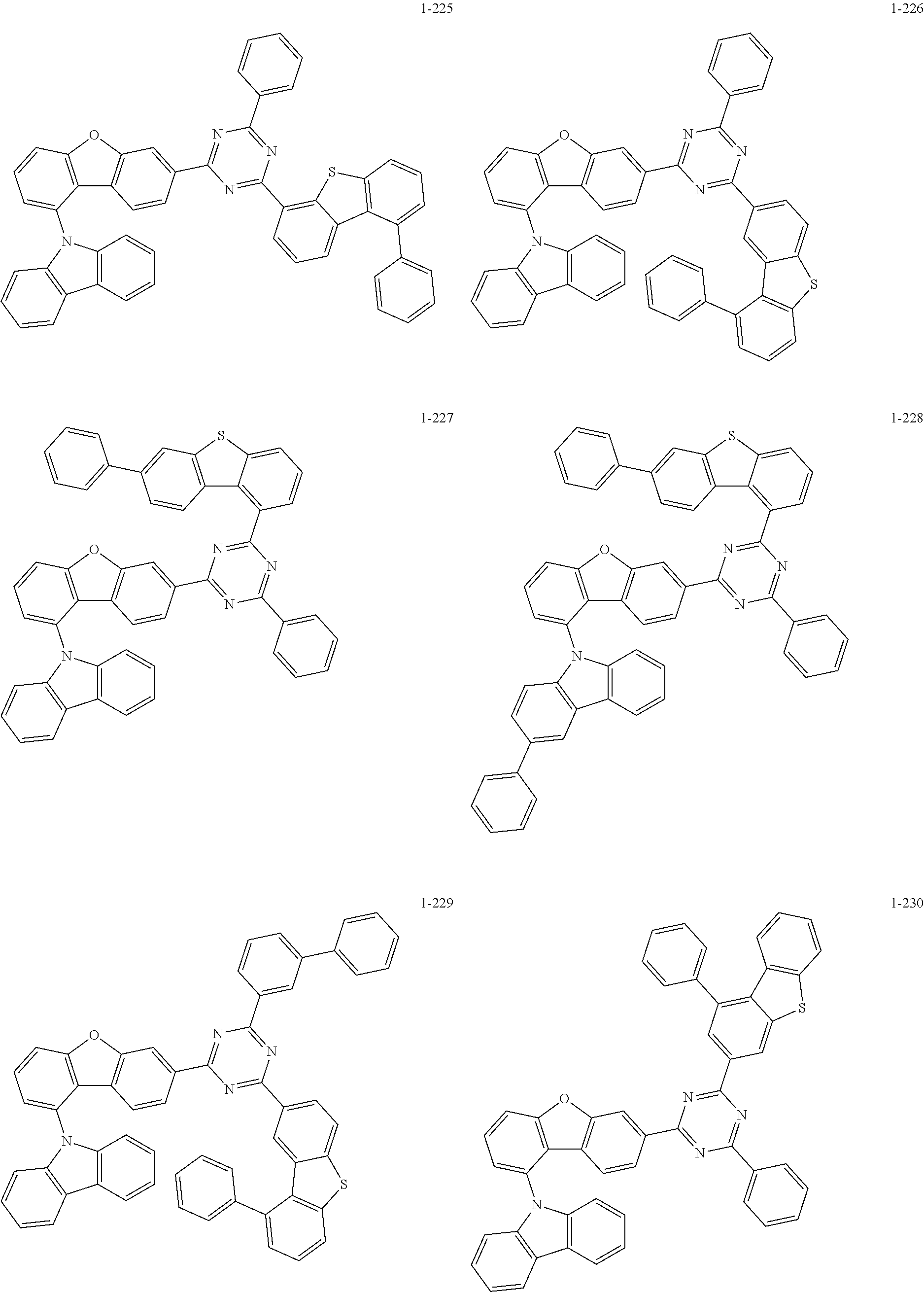

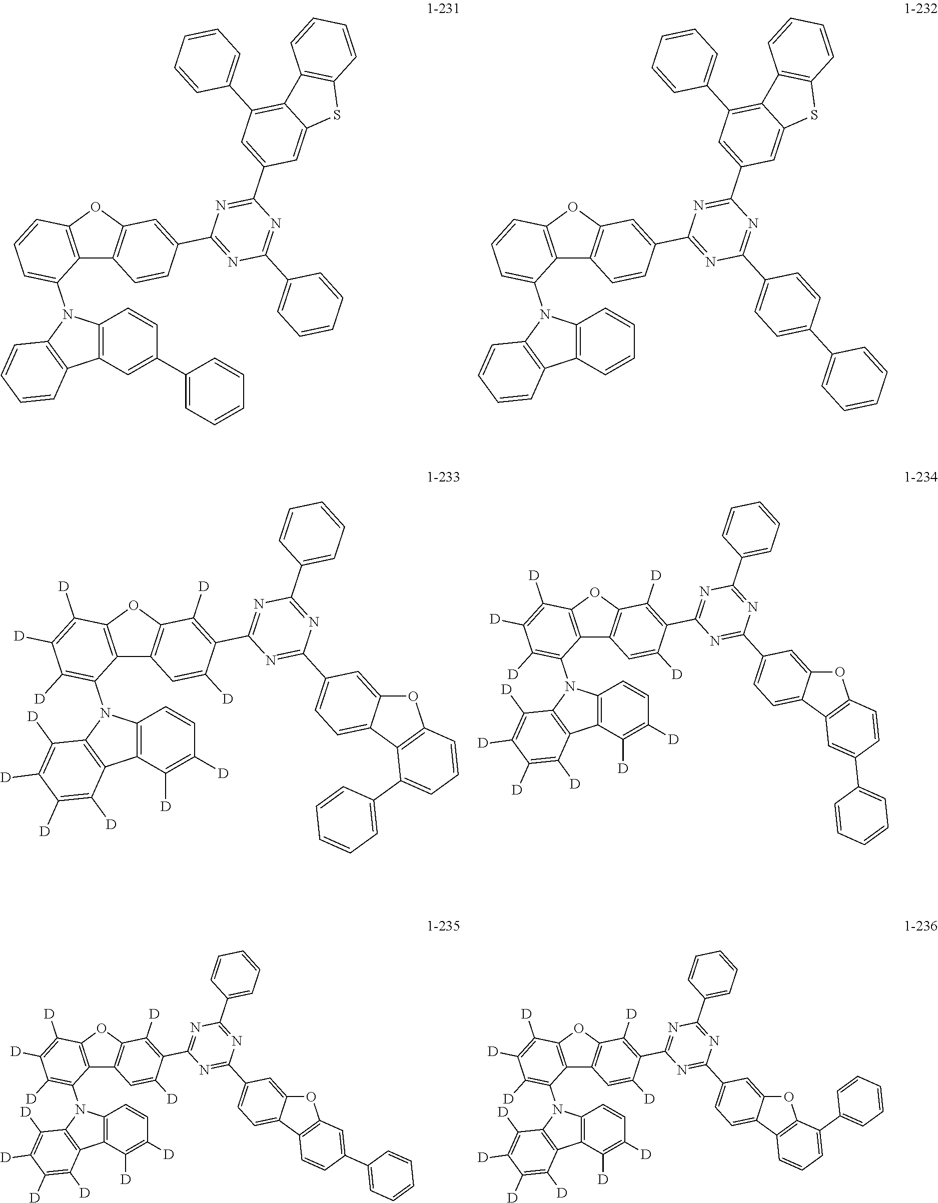

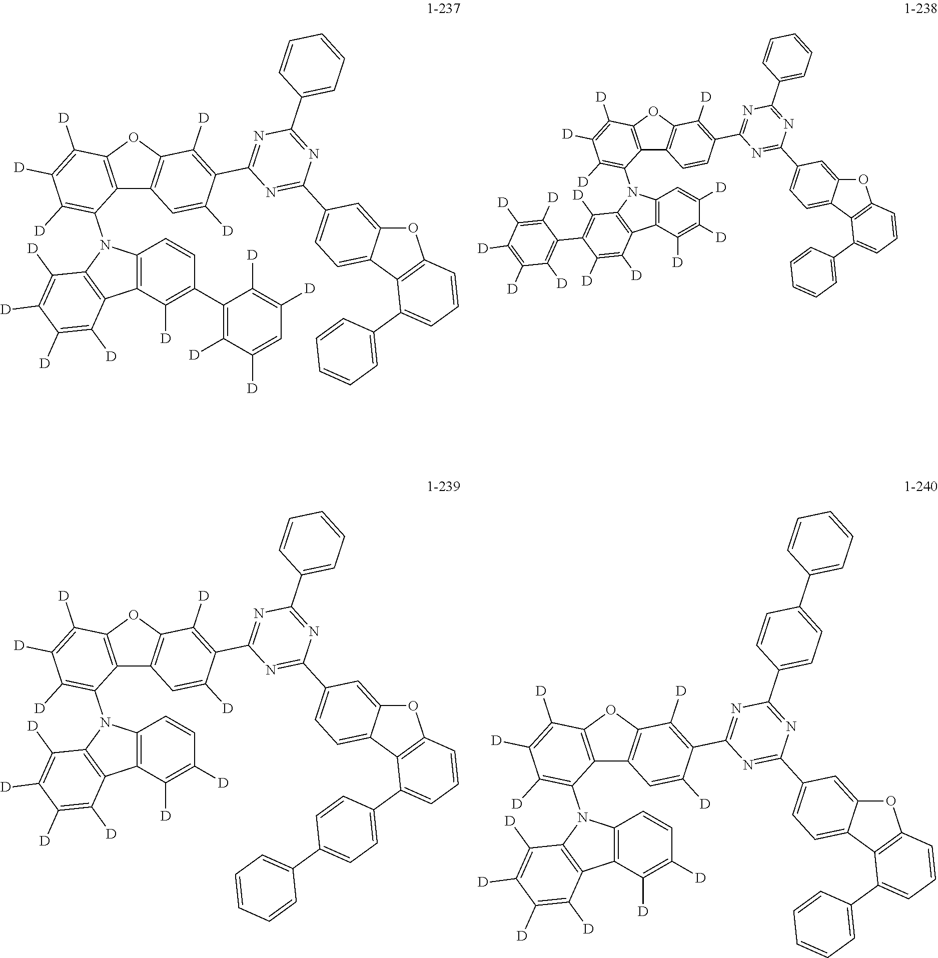

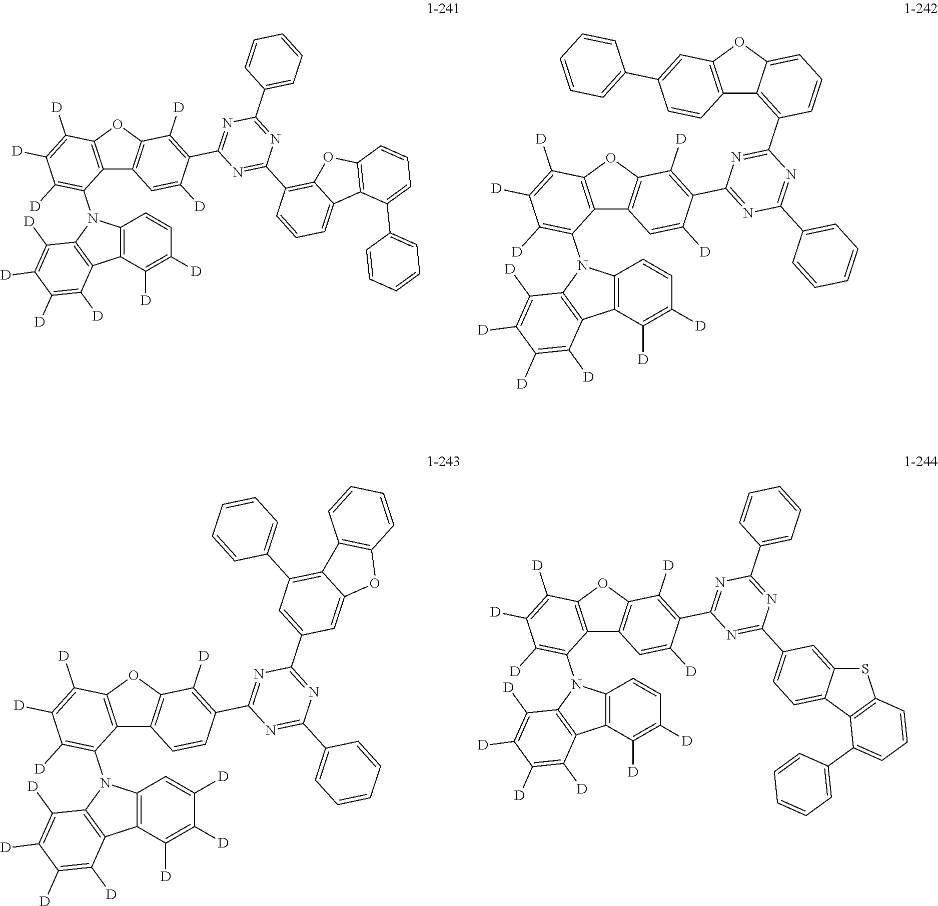

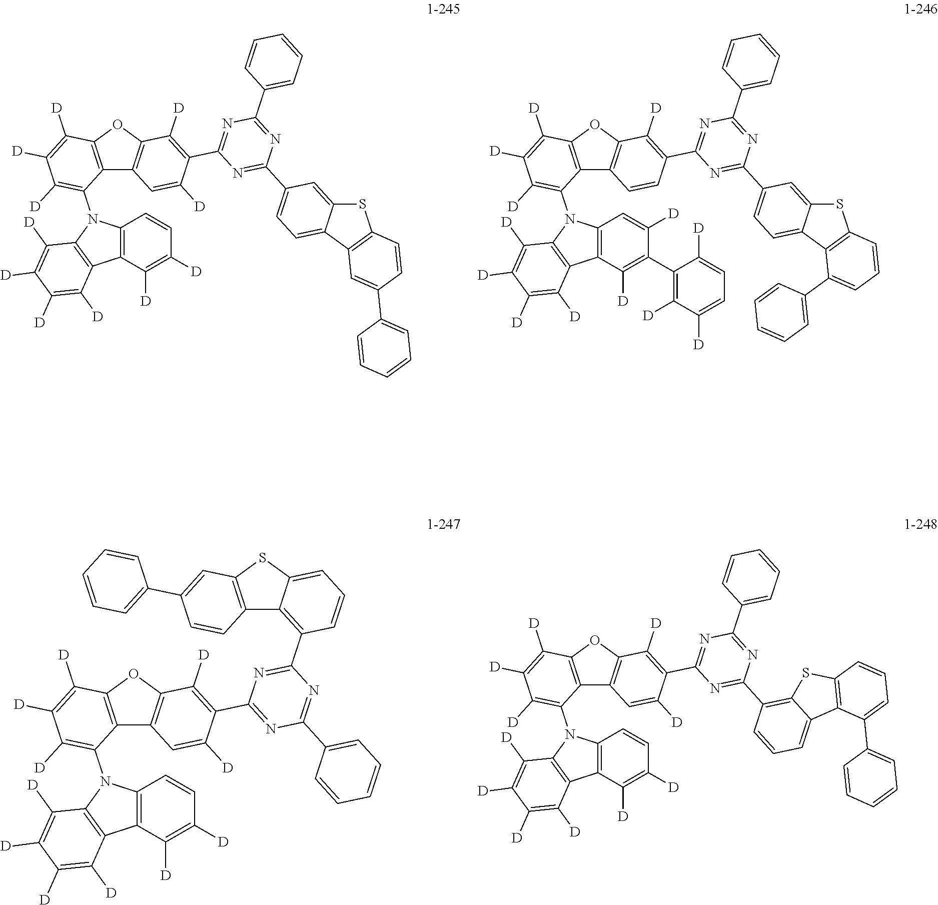

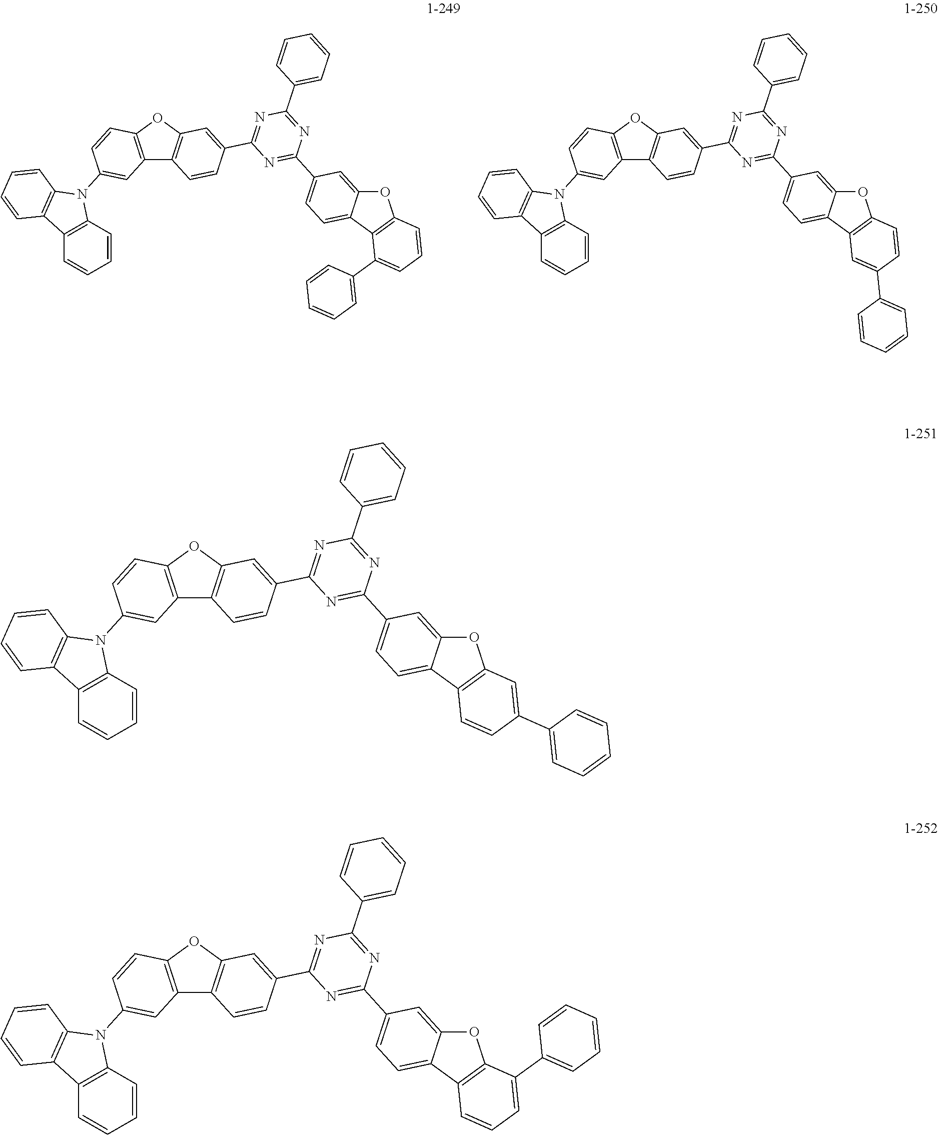 [SMILES: c1ccc(-c2nc(-c3ccc4c(c3)oc3ccc(-n5c6ccccc6c6ccccc65)cc34)nc(-c3ccc4c(c3)oc3cccc(-c5ccccc5)c34)n2)cc1 ; c1ccc(-c2ccc3oc4cc(-c5nc(-c6ccccc6)nc(-c6ccc7c(c6)oc6ccc(-n8c9ccccc9c9ccccc98)cc67)n5)ccc4c3c2)cc1 ; c1ccc(-c2ccc3c(c2)oc2cc(-c4nc(-c5ccccc5)nc(-c5ccc6c(c5)oc5ccc(-n7c8ccccc8c8ccccc87)cc56)n4)ccc23)cc1 ; c1ccc(-c2nc(-c3ccc4c(c3)oc3ccc(-n5c6ccccc6c6ccccc65)cc34)nc(-c3ccc4c(c3)oc3c(-c5ccccc5)cccc34)n2)cc1]

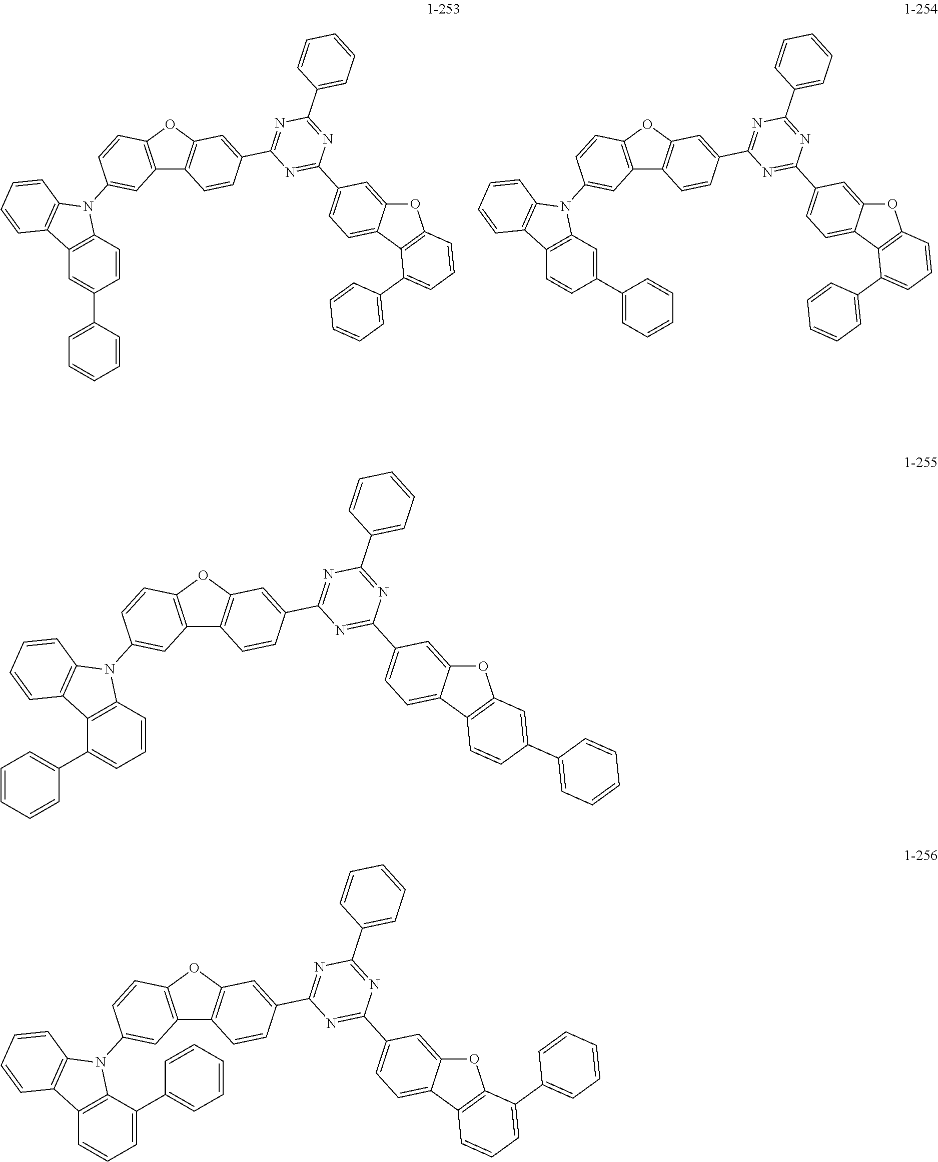

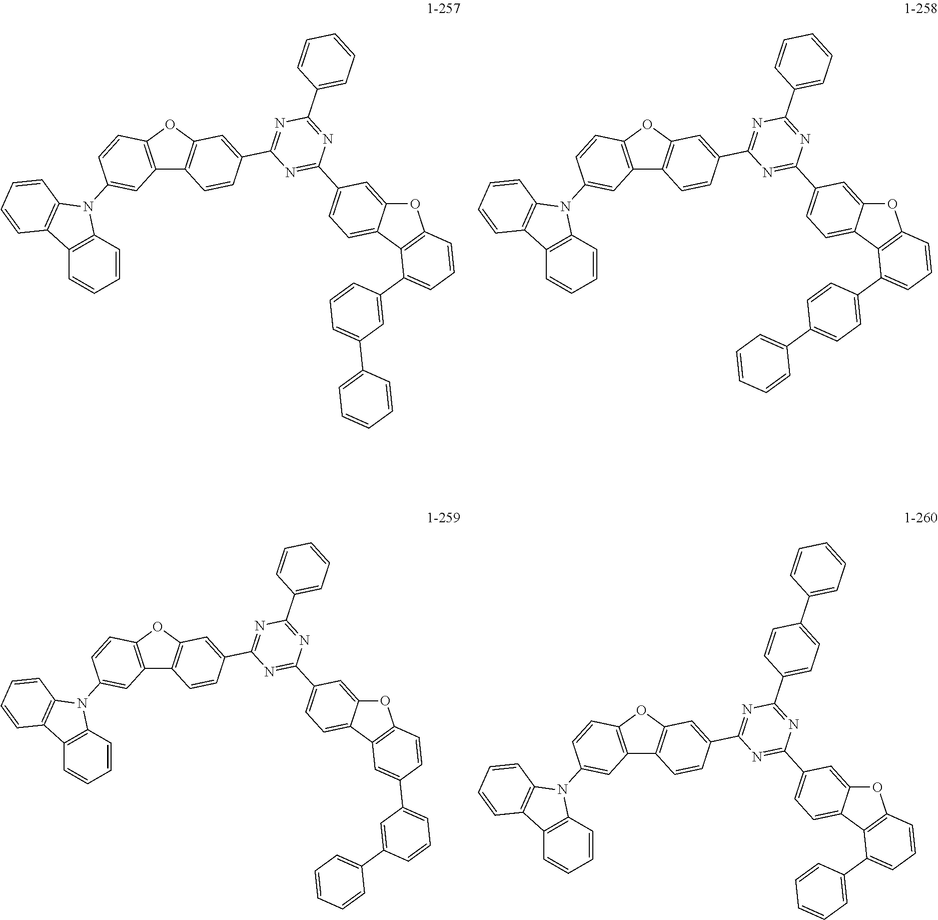 [SMILES: c1ccc(-c2cccc(-c3cccc4oc5cc(-c6nc(-c7ccccc7)nc(-c7ccc8c(c7)oc7ccc(-n9c%10ccccc%10c%10ccccc%109)cc78)n6)ccc5c34)c2)cc1 ; c1ccc(-c2ccc(-c3cccc4oc5cc(-c6nc(-c7ccccc7)nc(-c7ccc8c(c7)oc7ccc(-n9c%10ccccc%10c%10ccccc%109)cc78)n6)ccc5c34)cc2)cc1 ; c1ccc(-c2cccc(-c3ccc4oc5cc(-c6nc(-c7ccccc7)nc(-c7ccc8c(c7)oc7ccc(-n9c%10ccccc%10c%10ccccc%109)cc78)n6)ccc5c4c3)c2)cc1 ; c1ccc(-c2ccc(-c3nc(-c4ccc5c(c4)oc4ccc(-n6c7ccccc7c7ccccc76)cc45)nc(-c4ccc5c(c4)oc4cccc(-c6ccccc6)c45)n3)cc2)cc1]

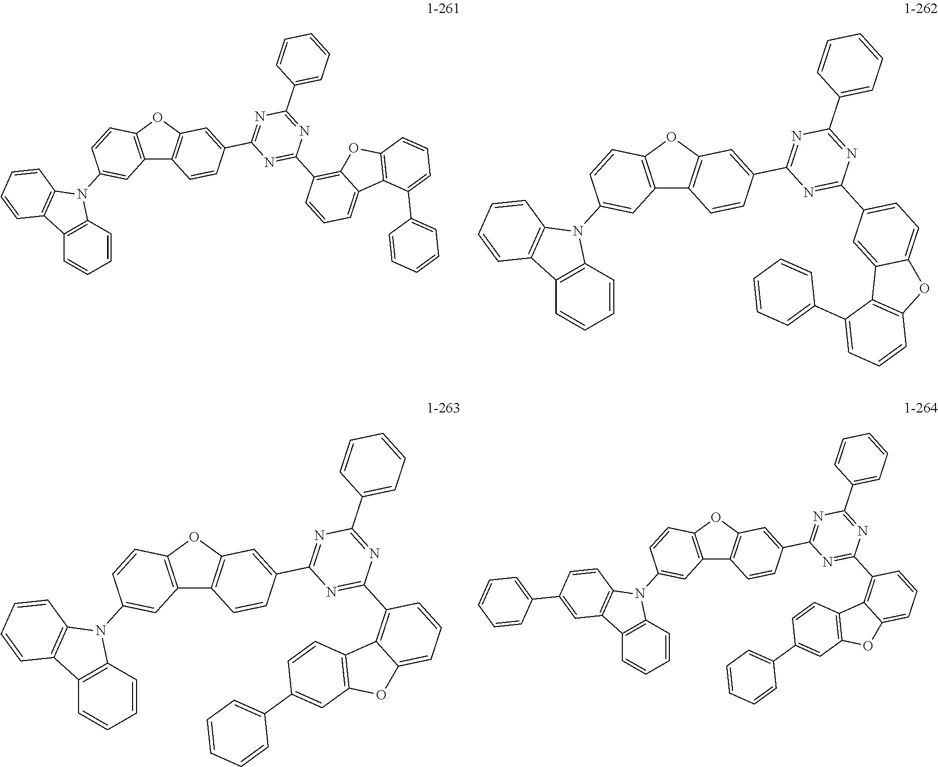 [SMILES: c1ccc(-c2nc(-c3ccc4c(c3)oc3ccc(-n5c6ccccc6c6ccccc65)cc34)nc(-c3cccc4c3oc3cccc(-c5ccccc5)c34)n2)cc1 ; c1ccc(-c2nc(-c3ccc4c(c3)oc3ccc(-n5c6ccccc6c6ccccc65)cc34)nc(-c3ccc4oc5cccc(-c6ccccc6)c5c4c3)n2)cc1 ; c1ccc(-c2ccc3c(c2)oc2cccc(-c4nc(-c5ccccc5)nc(-c5ccc6c(c5)oc5ccc(-n7c8ccccc8c8ccccc87)cc56)n4)c23)cc1 ; c1ccc(-c2ccc3c(c2)oc2cccc(-c4nc(-c5ccccc5)nc(-c5ccc6c(c5)oc5ccc(-n7c8ccccc8c8cc(-c9ccccc9)ccc87)cc56)n4)c23)cc1]

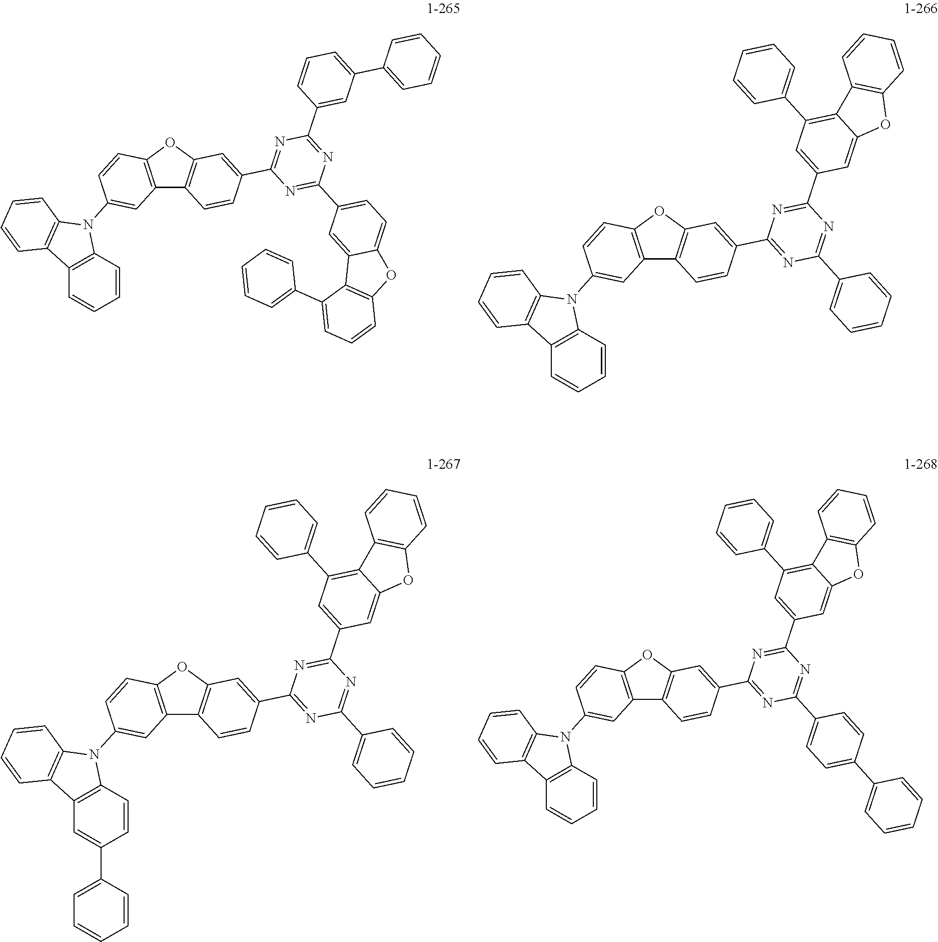 [SMILES: c1ccc(-c2cccc(-c3nc(-c4ccc5c(c4)oc4ccc(-n6c7ccccc7c7ccccc76)cc45)nc(-c4ccc5oc6cccc(-c7ccccc7)c6c5c4)n3)c2)cc1 ; c1ccc(-c2nc(-c3ccc4c(c3)oc3ccc(-n5c6ccccc6c6ccccc65)cc34)nc(-c3cc(-c4ccccc4)c4c(c3)oc3ccccc34)n2)cc1 ; c1ccc(-c2ccc3c(c2)c2ccccc2n3-c2ccc3oc4cc(-c5nc(-c6ccccc6)nc(-c6cc(-c7ccccc7)c7c(c6)oc6ccccc67)n5)ccc4c3c2)cc1 ; c1ccc(-c2ccc(-c3nc(-c4ccc5c(c4)oc4ccc(-n6c7ccccc7c7ccccc76)cc45)nc(-c4cc(-c5ccccc5)c5c(c4)oc4ccccc45)n3)cc2)cc1]

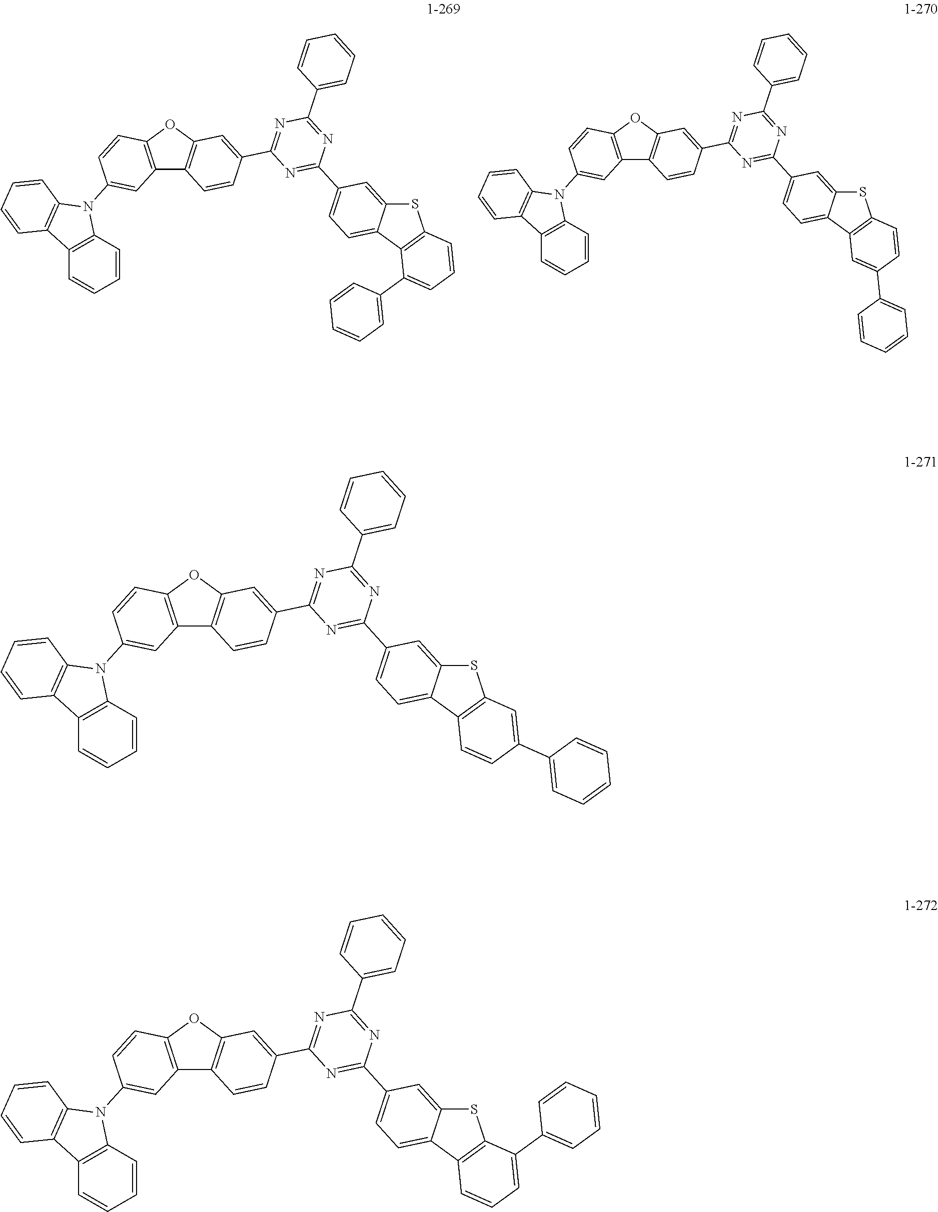 [SMILES: c1ccc(-c2nc(-c3ccc4c(c3)oc3ccc(-n5c6ccccc6c6ccccc65)cc34)nc(-c3ccc4c(c3)sc3cccc(-c5ccccc5)c34)n2)cc1 ; c1ccc(-c2ccc3sc4cc(-c5nc(-c6ccccc6)nc(-c6ccc7c(c6)oc6ccc(-n8c9ccccc9c9ccccc98)cc67)n5)ccc4c3c2)cc1 ; c1ccc(-c2ccc3c(c2)sc2cc(-c4nc(-c5ccccc5)nc(-c5ccc6c(c5)oc5ccc(-n7c8ccccc8c8ccccc87)cc56)n4)ccc23)cc1 ; c1ccc(-c2nc(-c3ccc4c(c3)oc3ccc(-n5c6ccccc6c6ccccc65)cc34)nc(-c3ccc4c(c3)sc3c(-c5ccccc5)cccc34)n2)cc1]

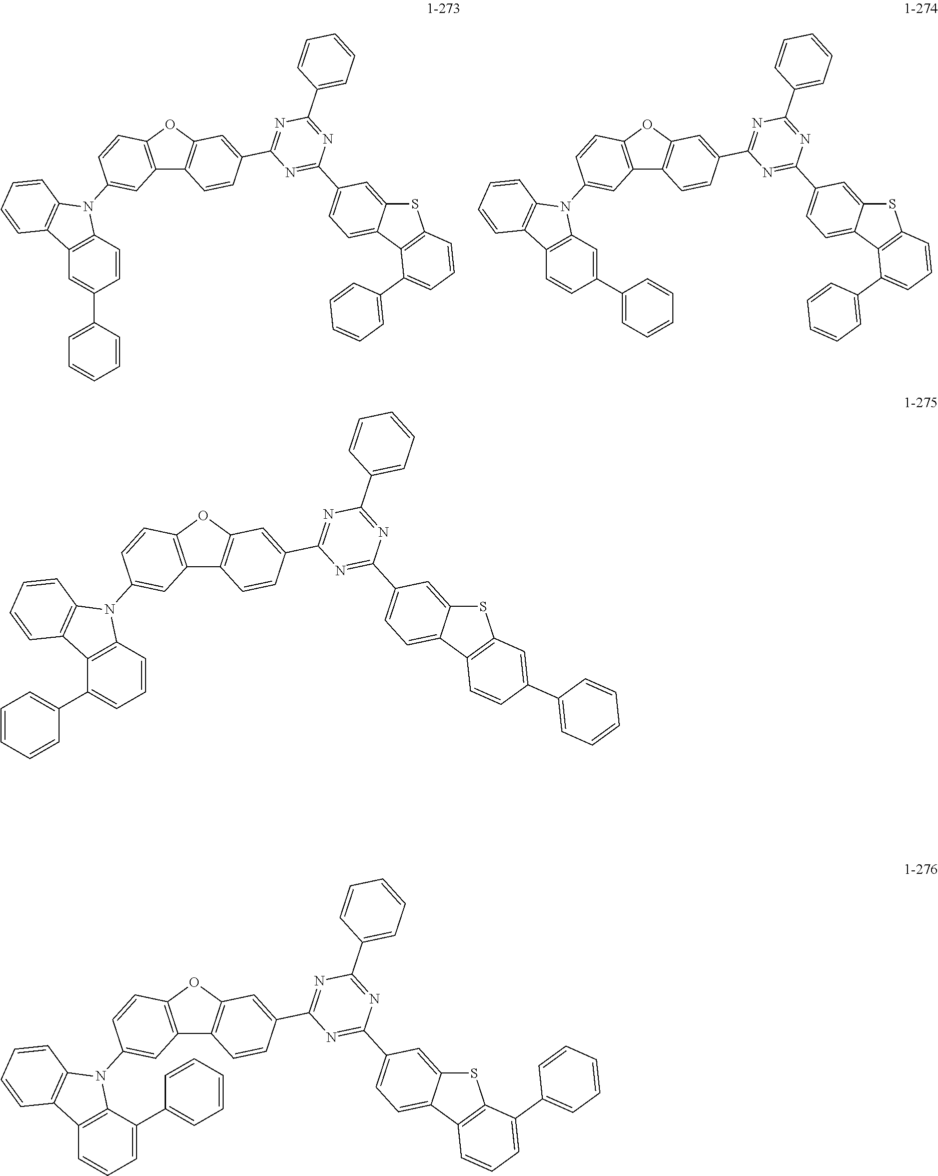

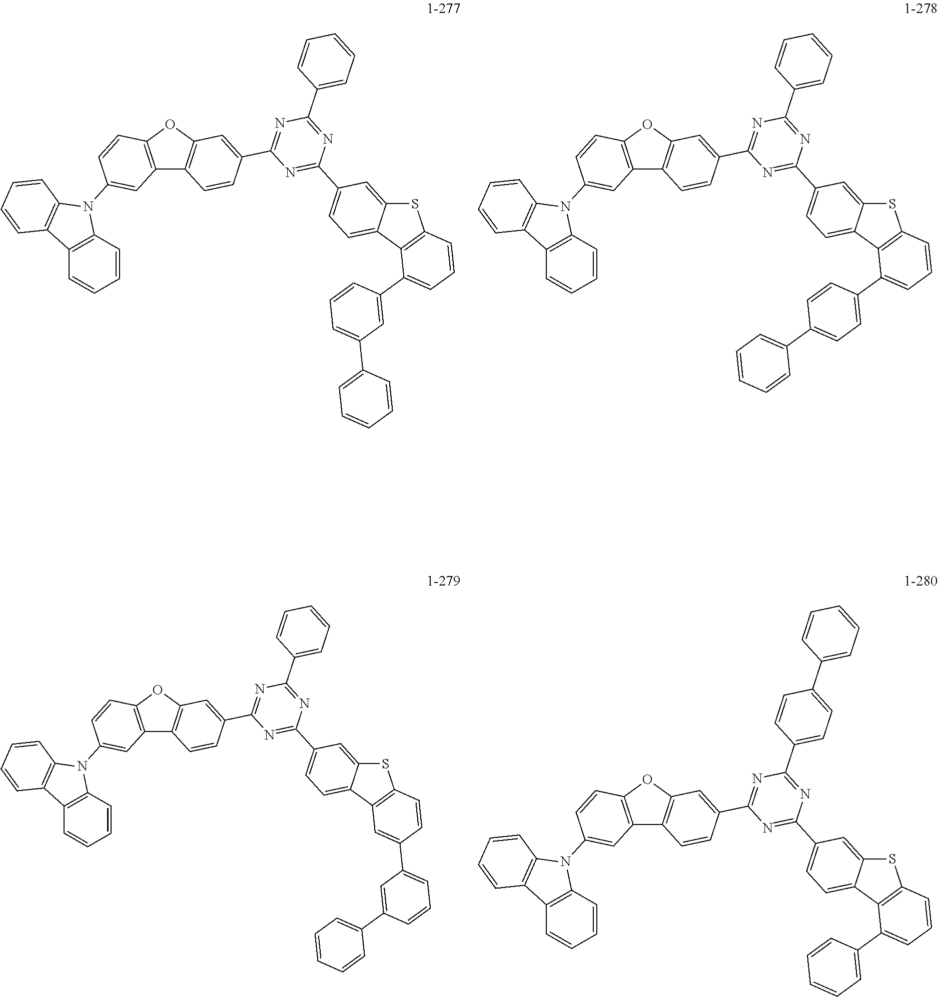 [SMILES: c1ccc(-c2cccc(-c3cccc4sc5cc(-c6nc(-c7ccccc7)nc(-c7ccc8c(c7)oc7ccc(-n9c%10ccccc%10c%10ccccc%109)cc78)n6)ccc5c34)c2)cc1 ; c1ccc(-c2ccc(-c3cccc4sc5cc(-c6nc(-c7ccccc7)nc(-c7ccc8c(c7)oc7ccc(-n9c%10ccccc%10c%10ccccc%109)cc78)n6)ccc5c34)cc2)cc1 ; c1ccc(-c2cccc(-c3ccc4sc5cc(-c6nc(-c7ccccc7)nc(-c7ccc8c(c7)oc7ccc(-n9c%10ccccc%10c%10ccccc%109)cc78)n6)ccc5c4c3)c2)cc1 ; c1ccc(-c2ccc(-c3nc(-c4ccc5c(c4)oc4ccc(-n6c7ccccc7c7ccccc76)cc45)nc(-c4ccc5c(c4)sc4cccc(-c6ccccc6)c45)n3)cc2)cc1]

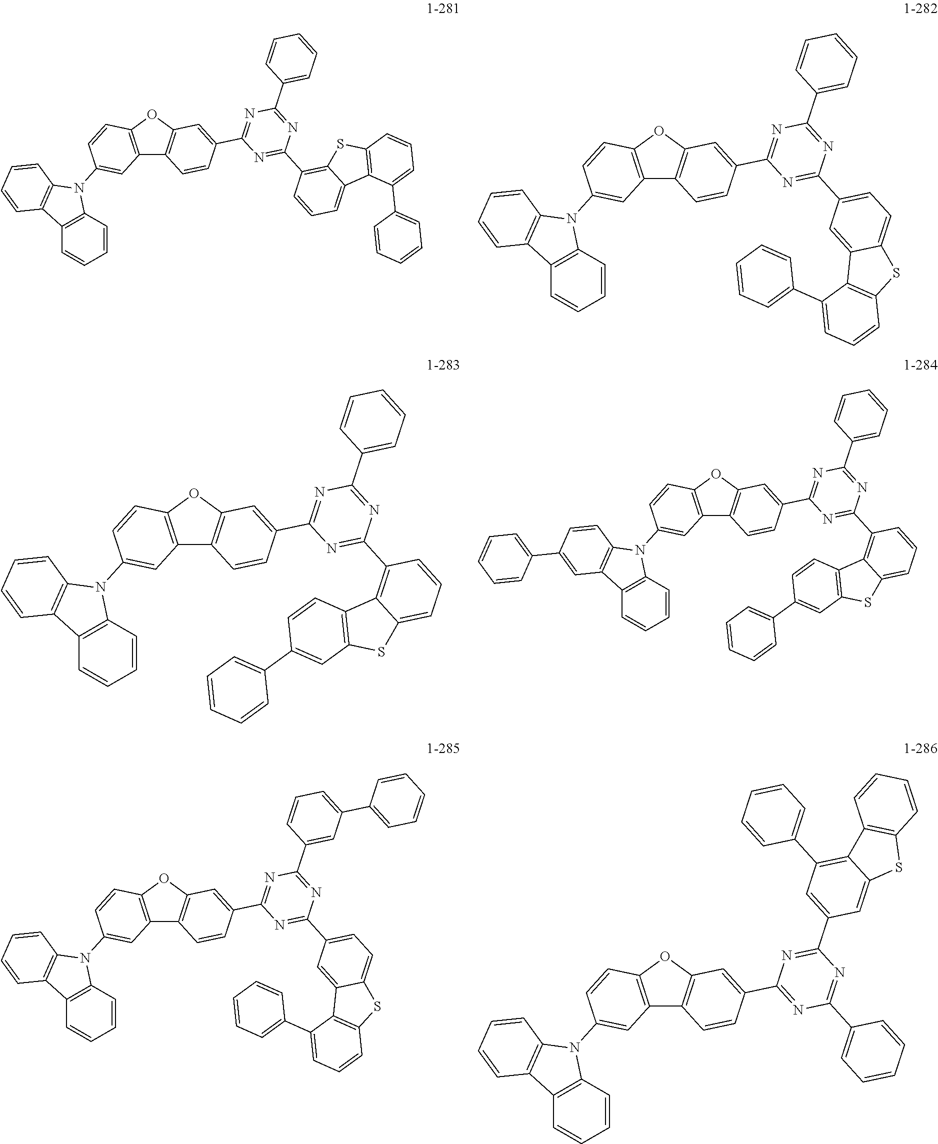

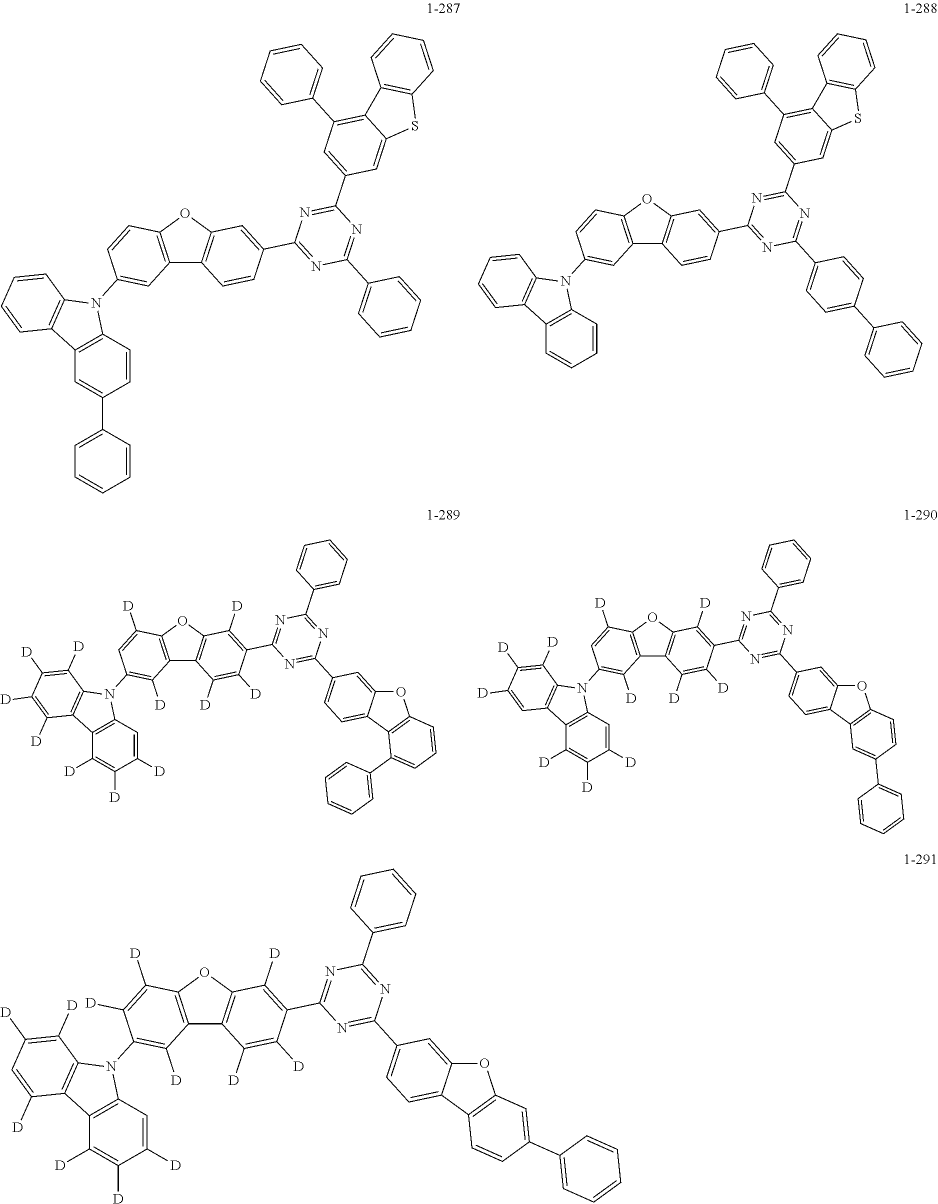

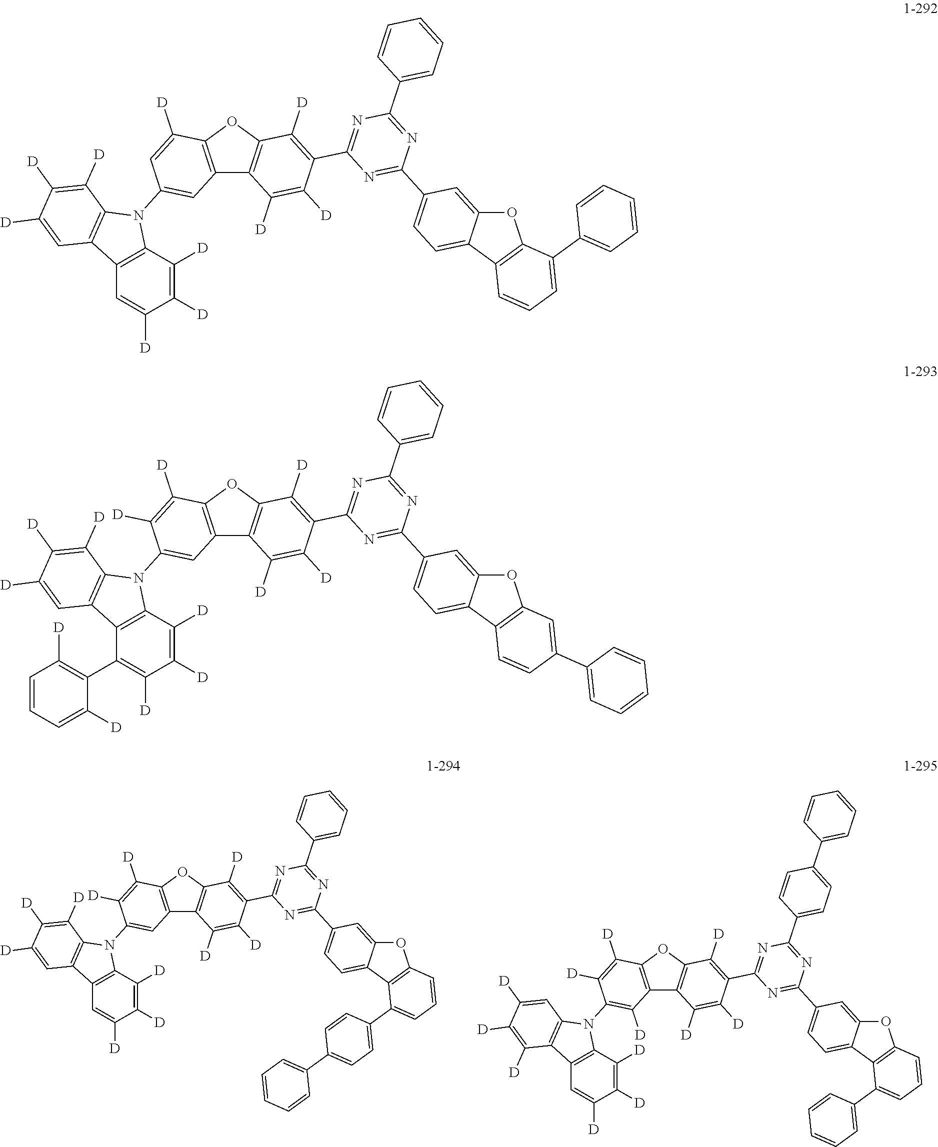

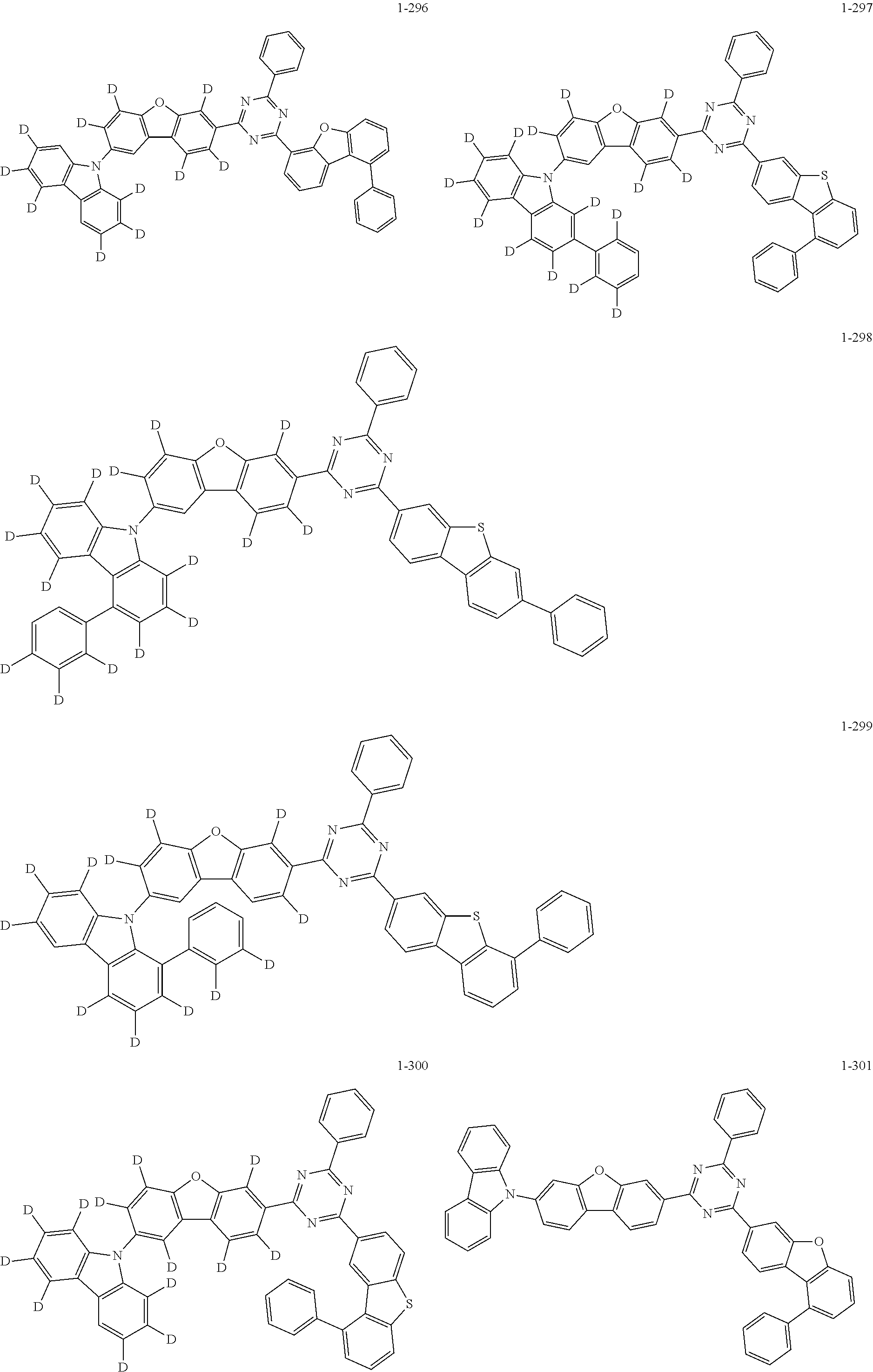

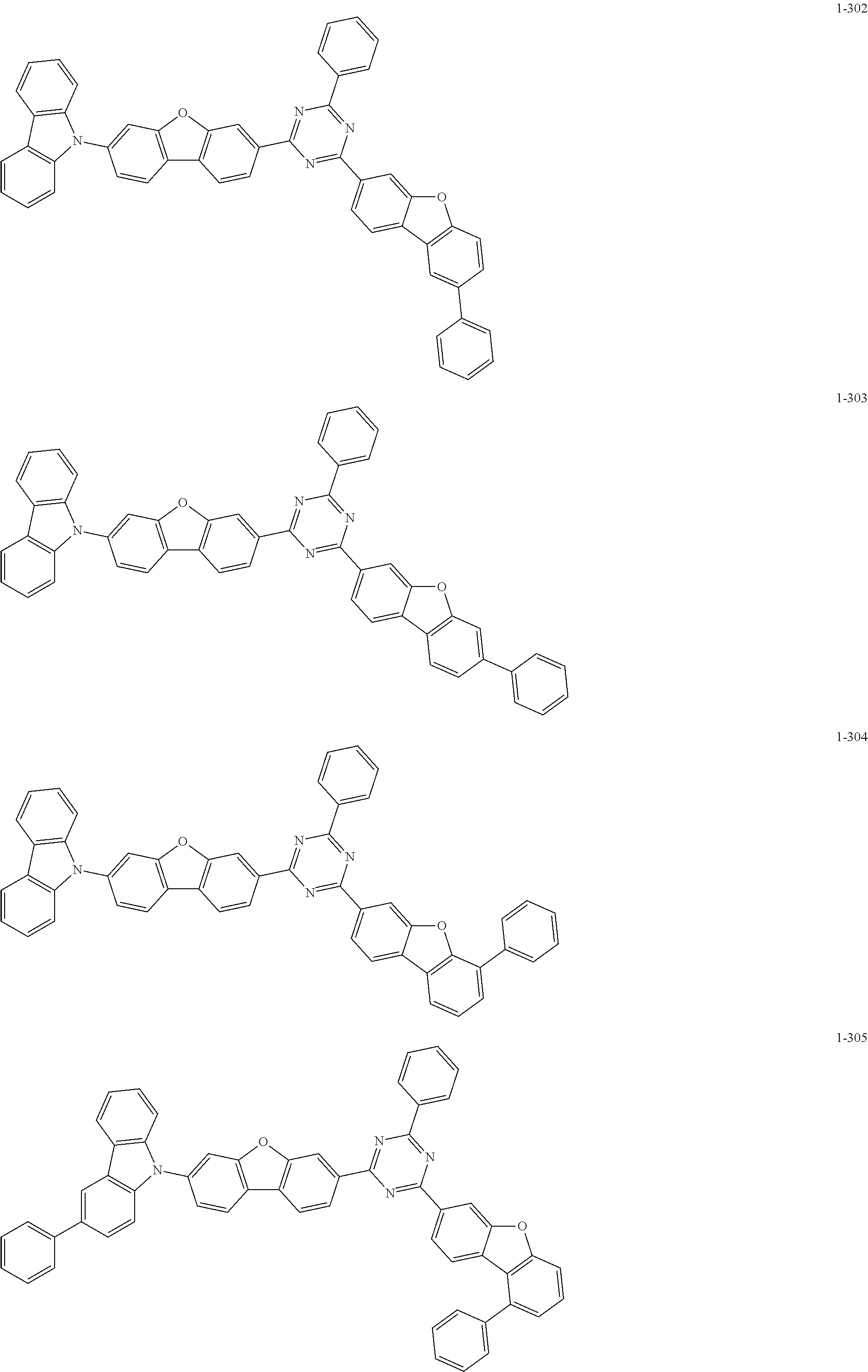

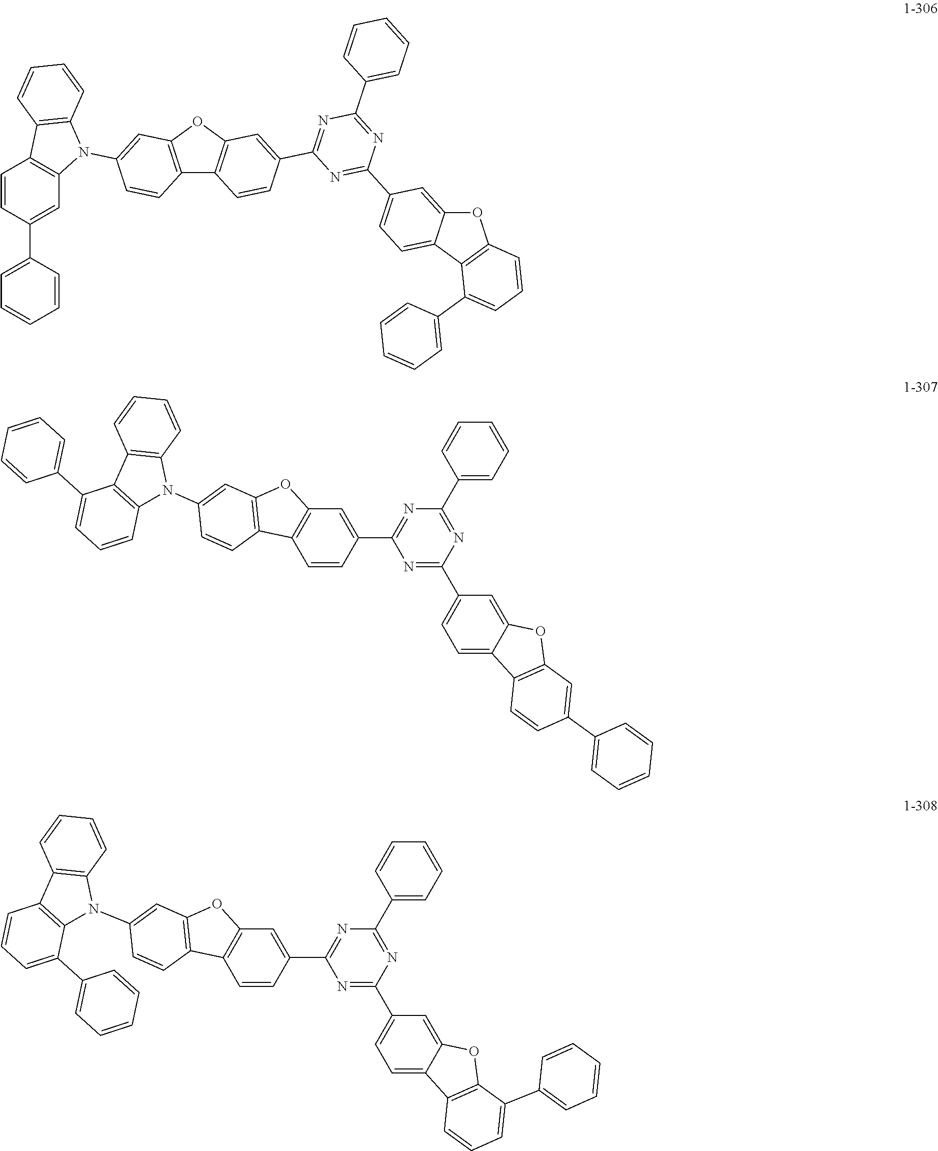 [SMILES: c1ccc(-c2ccc3c4ccccc4n(-c4ccc5c(c4)oc4cc(-c6nc(-c7ccccc7)nc(-c7ccc8c(c7)oc7cccc(-c9ccccc9)c78)n6)ccc45)c3c2)cc1 ; c1ccc(-c2ccc3c(c2)oc2cc(-c4nc(-c5ccccc5)nc(-c5ccc6c(c5)oc5cc(-n7c8ccccc8c8c(-c9ccccc9)cccc87)ccc56)n4)ccc23)cc1 ; c1ccc(-c2nc(-c3ccc4c(c3)oc3cc(-n5c6ccccc6c6cccc(-c7ccccc7)c65)ccc34)nc(-c3ccc4c(c3)oc3c(-c5ccccc5)cccc34)n2)cc1]

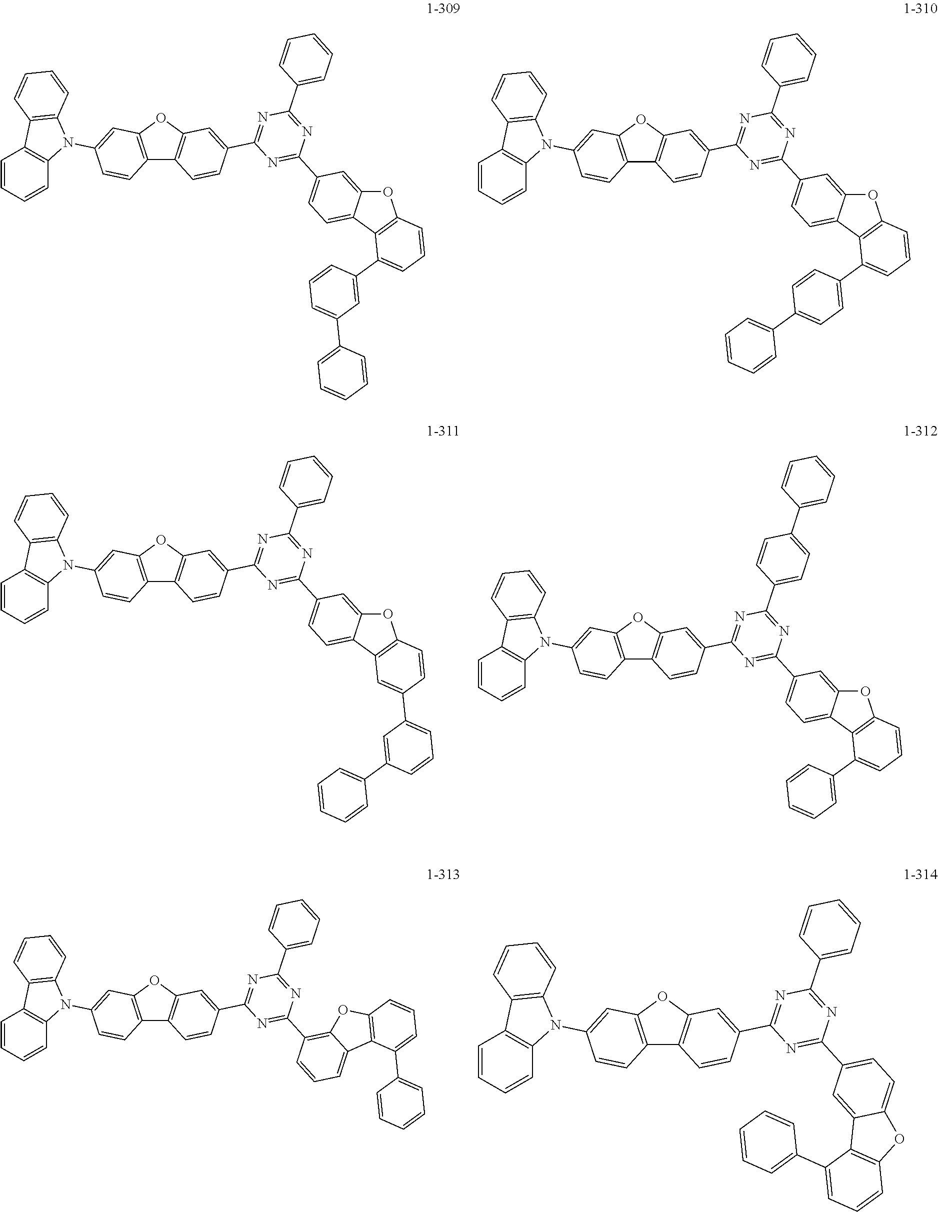

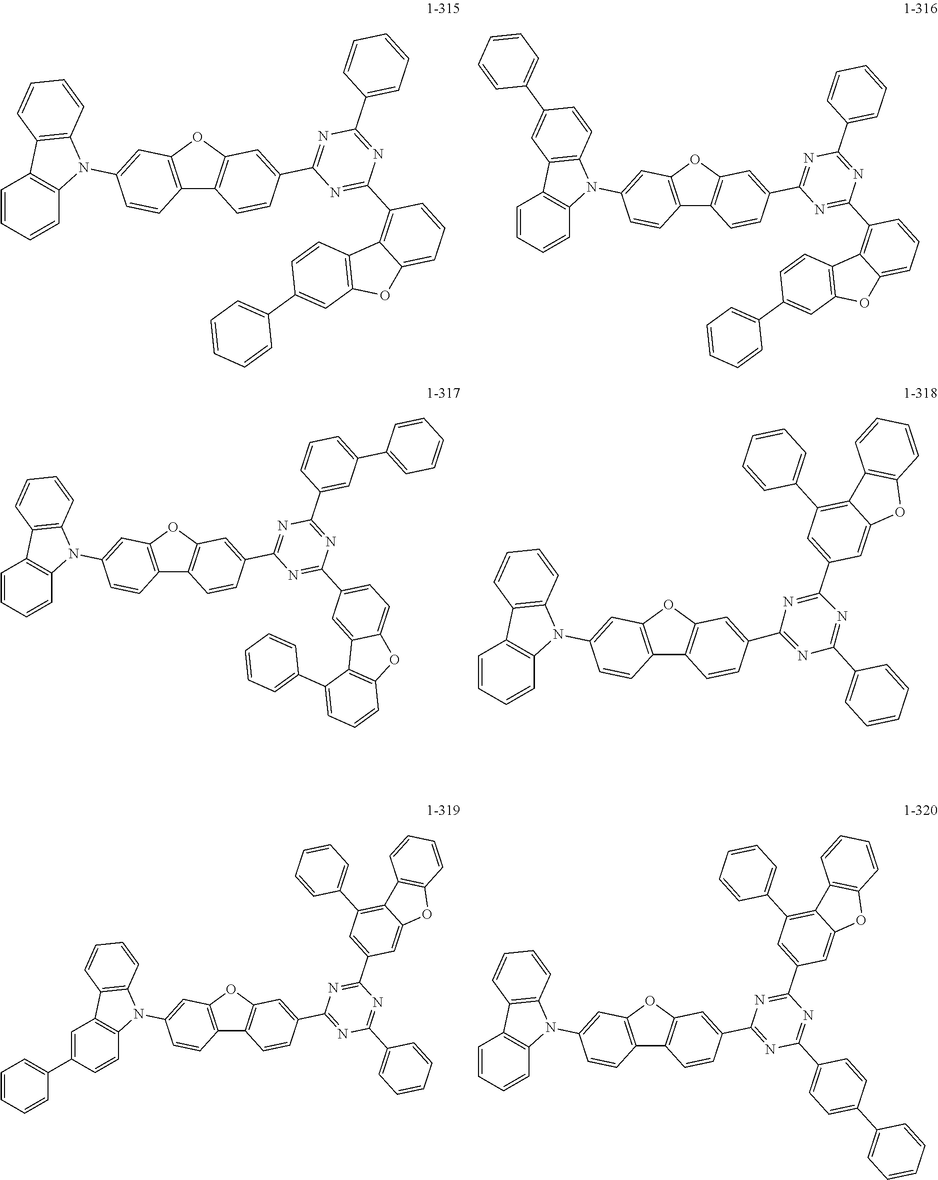

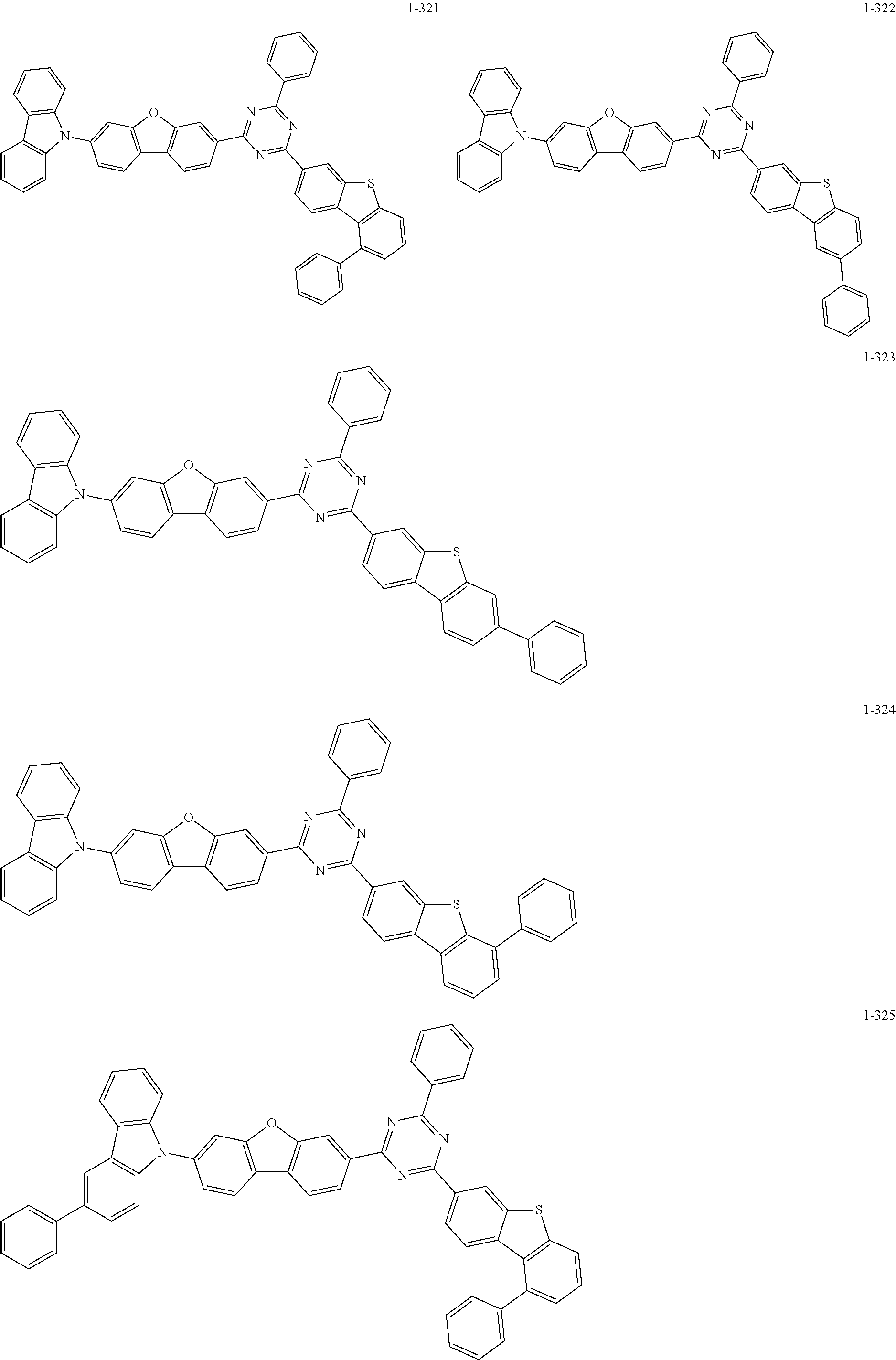

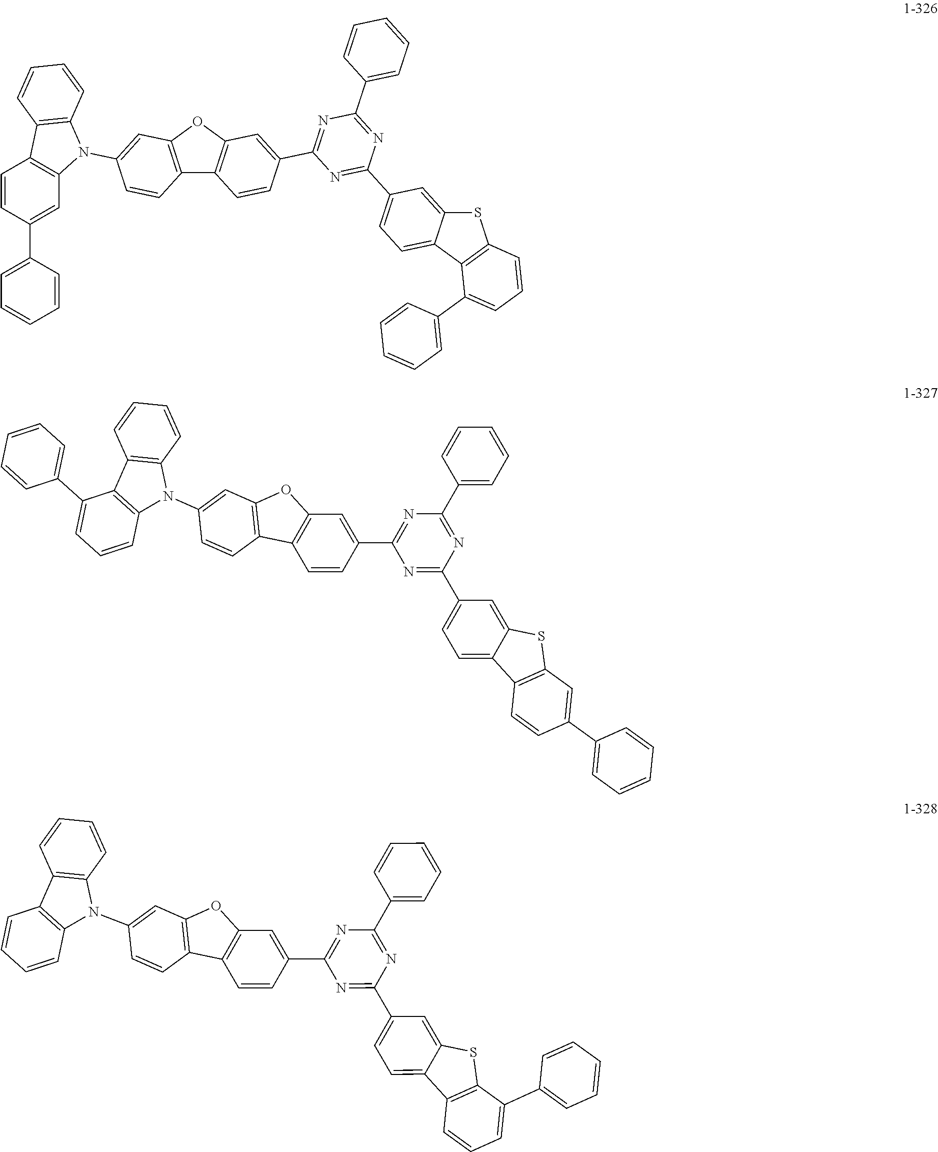 [SMILES: c1ccc(-c2ccc3c4ccccc4n(-c4ccc5c(c4)oc4cc(-c6nc(-c7ccccc7)nc(-c7ccc8c(c7)sc7cccc(-c9ccccc9)c78)n6)ccc45)c3c2)cc1 ; c1ccc(-c2ccc3c(c2)sc2cc(-c4nc(-c5ccccc5)nc(-c5ccc6c(c5)oc5cc(-n7c8ccccc8c8c(-c9ccccc9)cccc87)ccc56)n4)ccc23)cc1 ; c1ccc(-c2nc(-c3ccc4c(c3)oc3cc(-n5c6ccccc6c6ccccc65)ccc34)nc(-c3ccc4c(c3)sc3c(-c5ccccc5)cccc34)n2)cc1]

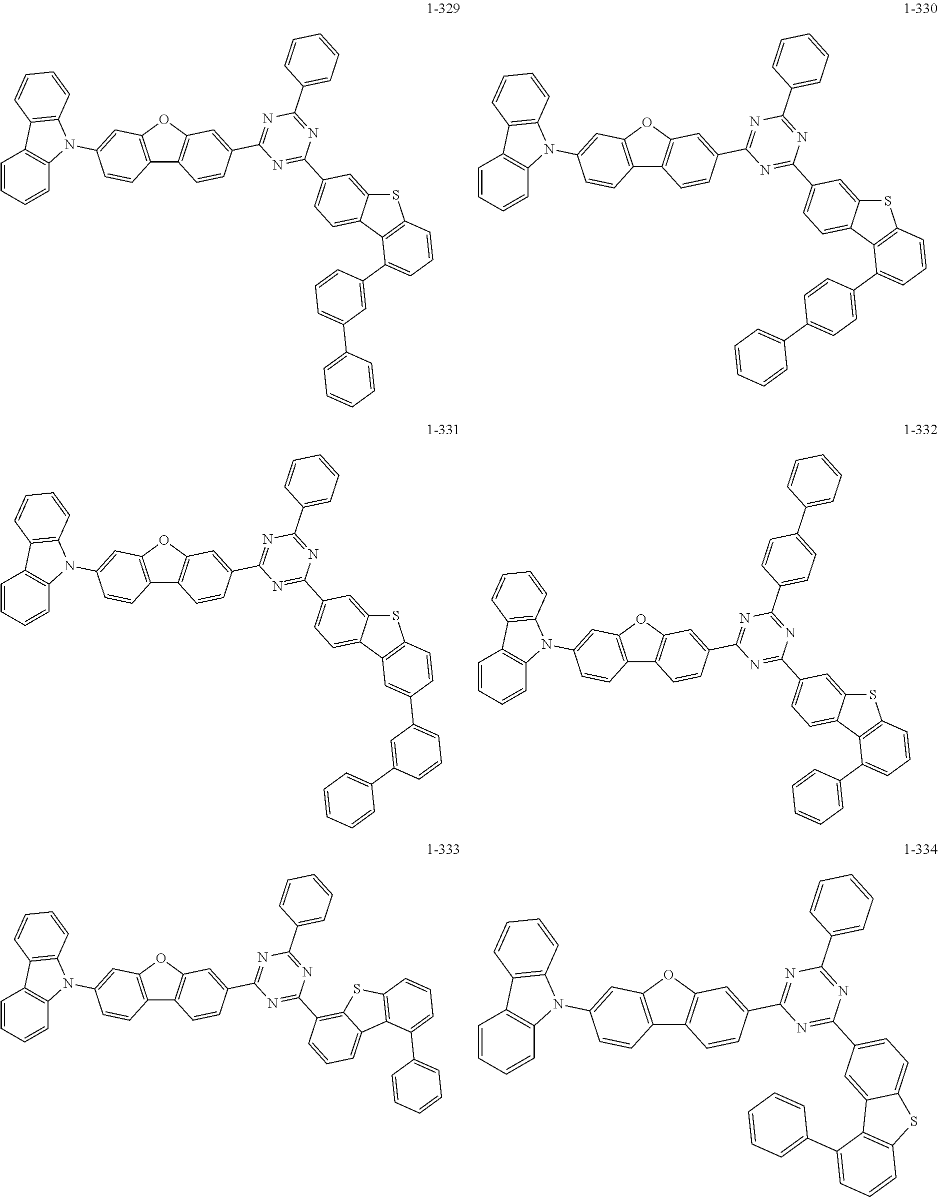

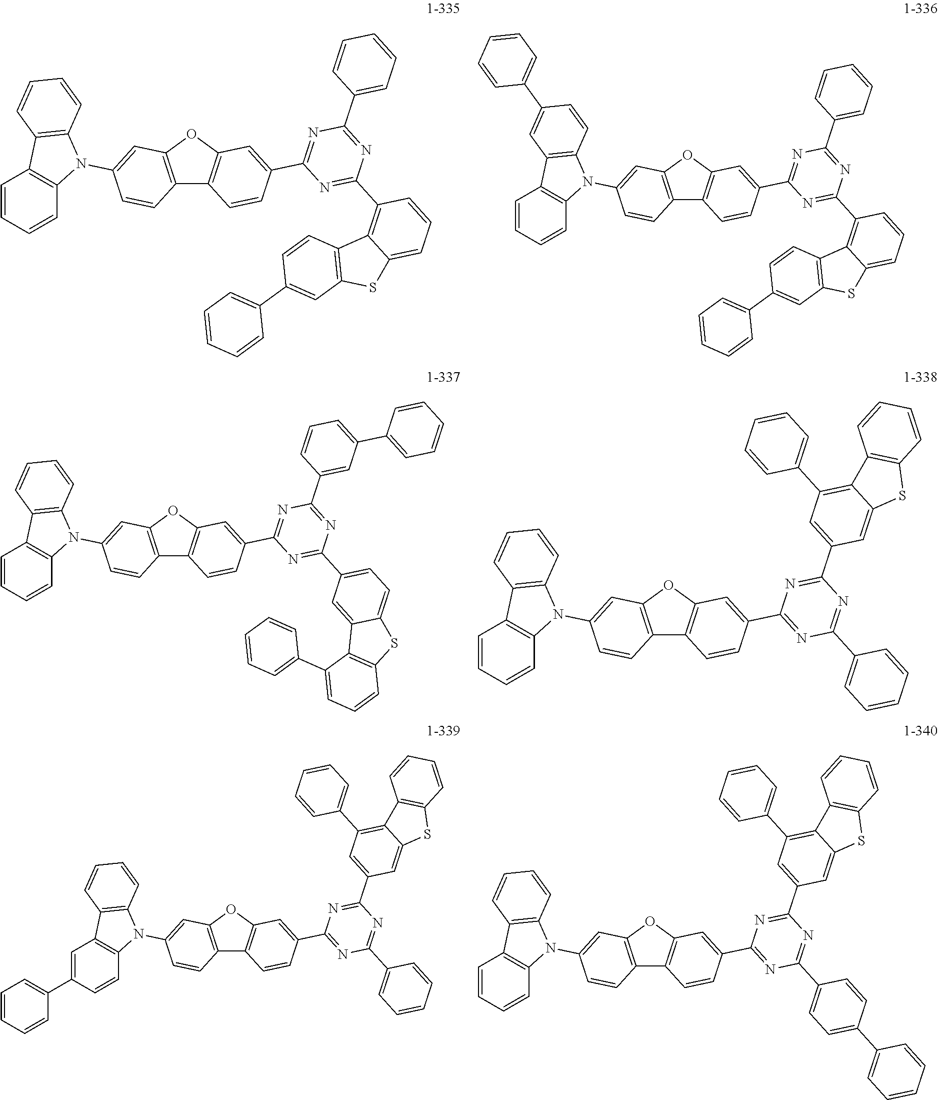

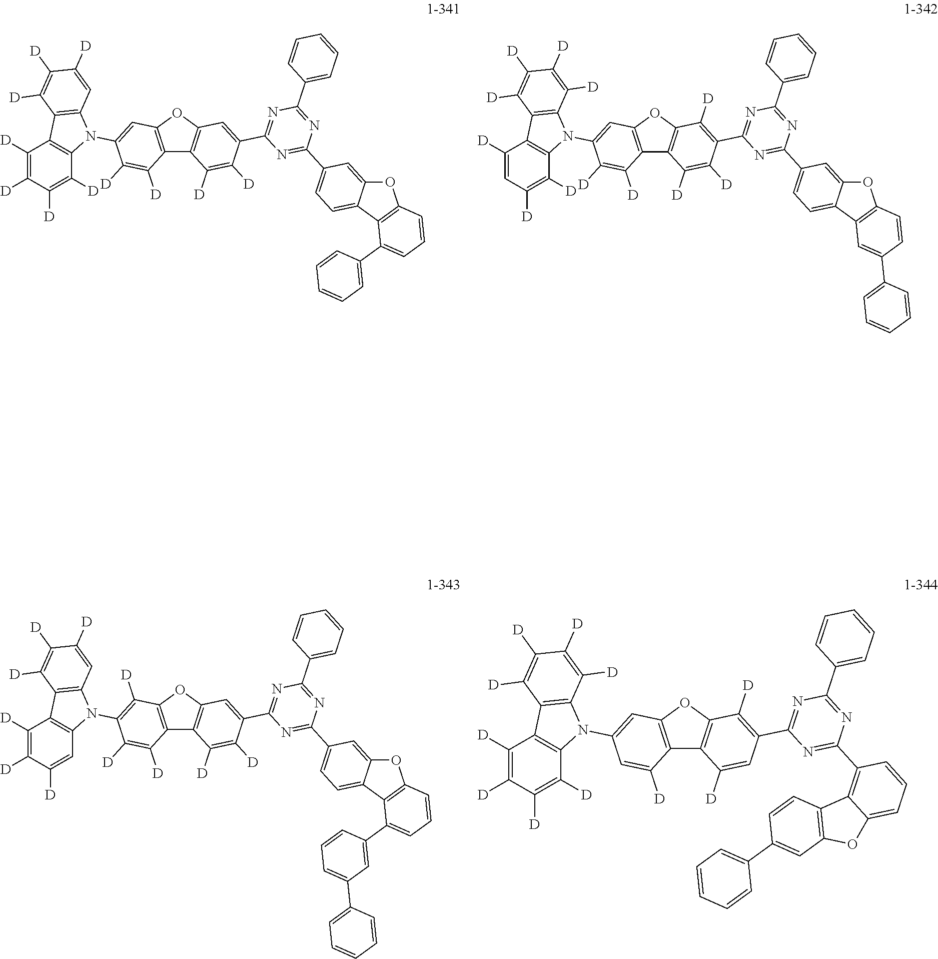

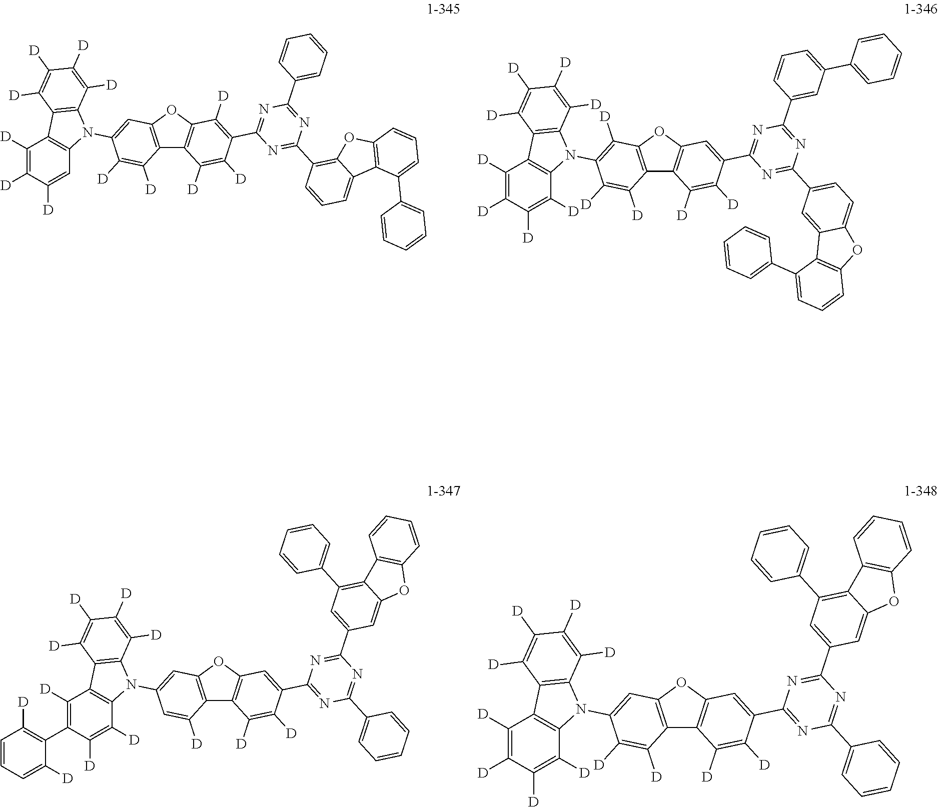

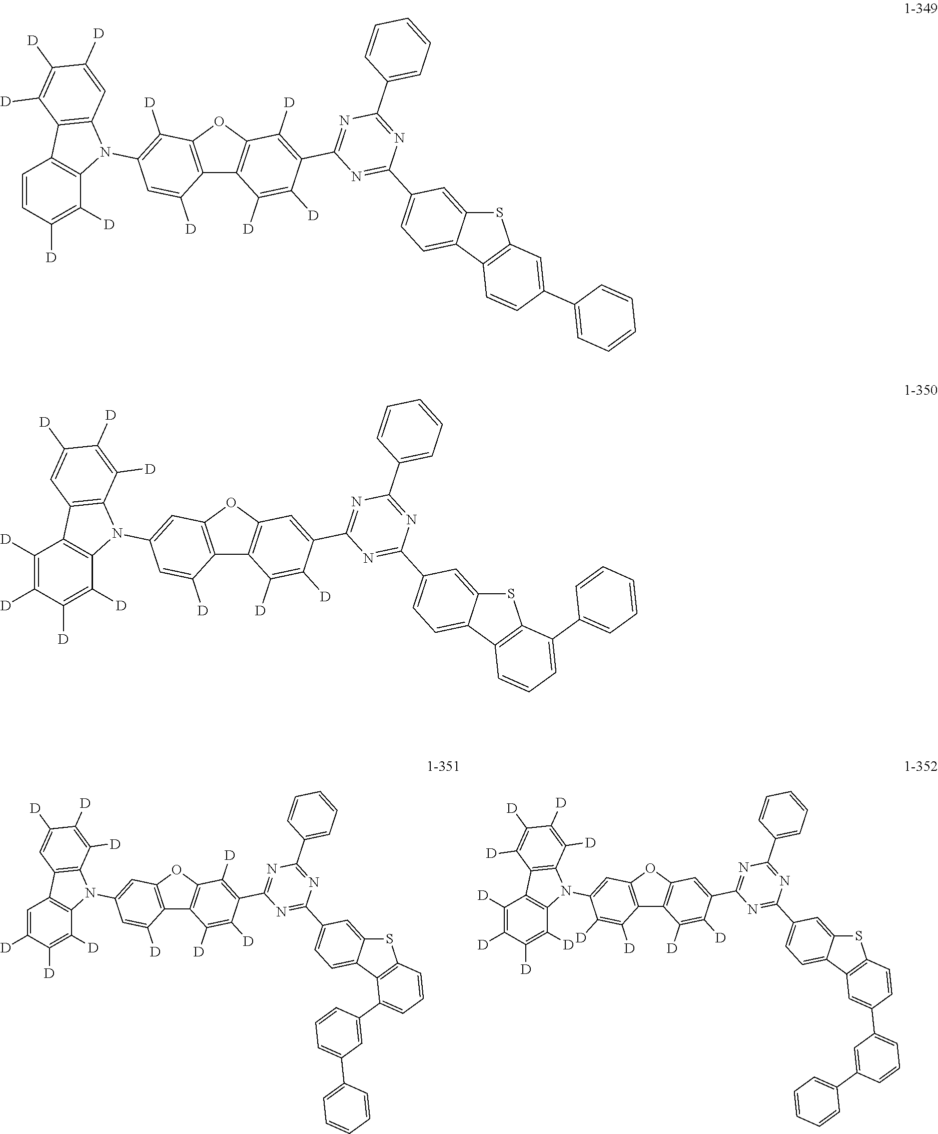

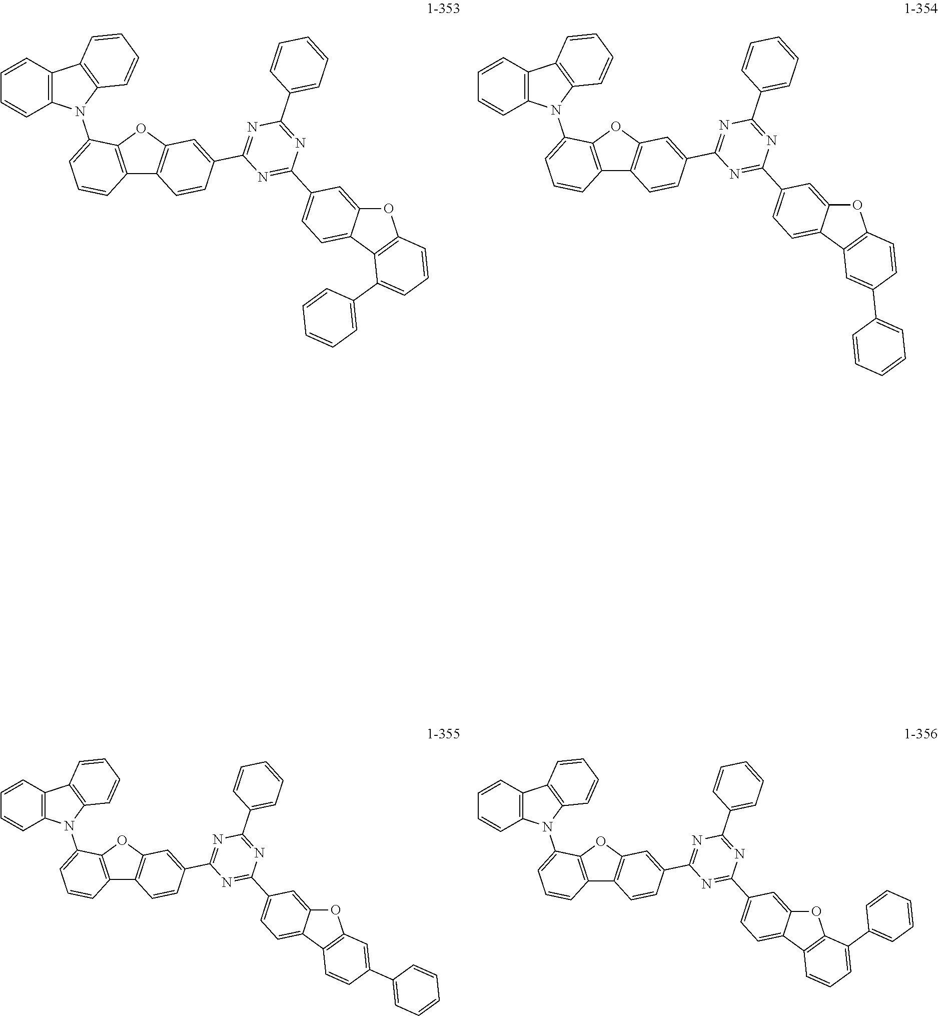 [SMILES: c1ccc(-c2nc(-c3ccc4c(c3)oc3c(-n5c6ccccc6c6ccccc65)cccc34)nc(-c3ccc4c(c3)oc3cccc(-c5ccccc5)c34)n2)cc1 ; c1ccc(-c2ccc3oc4cc(-c5nc(-c6ccccc6)nc(-c6ccc7c(c6)oc6c(-n8c9ccccc9c9ccccc98)cccc67)n5)ccc4c3c2)cc1 ; c1ccc(-c2ccc3c(c2)oc2cc(-c4nc(-c5ccccc5)nc(-c5ccc6c(c5)oc5c(-n7c8ccccc8c8ccccc87)cccc56)n4)ccc23)cc1 ; c1ccc(-c2nc(-c3ccc4c(c3)oc3c(-c5ccccc5)cccc34)nc(-c3ccc4c(c3)oc3c(-n5c6ccccc6c6ccccc65)cccc34)n2)cc1]

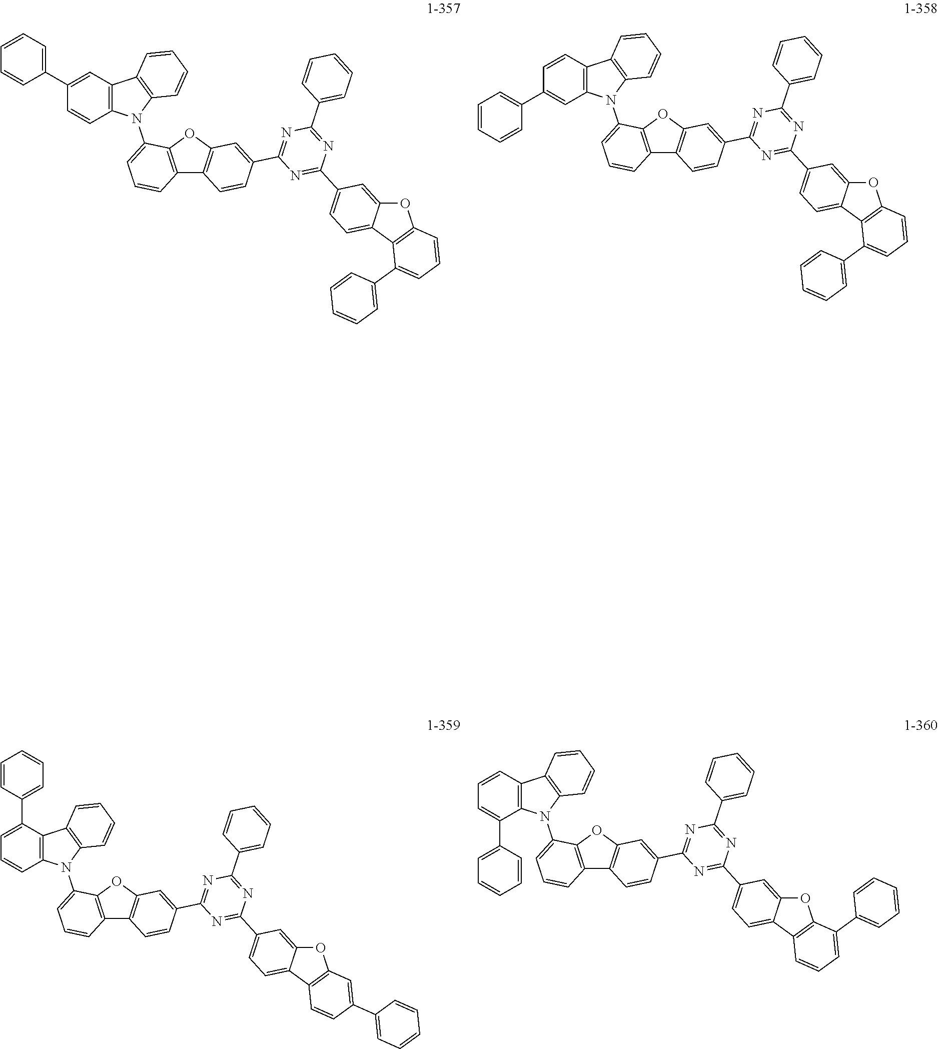 [SMILES: c1ccc(-c2ccc3c(c2)c2ccccc2n3-c2cccc3c2oc2cc(-c4nc(-c5ccccc5)nc(-c5ccc6c(c5)oc5cccc(-c7ccccc7)c56)n4)ccc23)cc1 ; c1ccc(-c2ccc3c4ccccc4n(-c4cccc5c4oc4cc(-c6nc(-c7ccccc7)nc(-c7ccc8c(c7)oc7cccc(-c9ccccc9)c78)n6)ccc45)c3c2)cc1 ; c1ccc(-c2ccc3c(c2)oc2cc(-c4nc(-c5ccccc5)nc(-c5ccc6c(c5)oc5c(-n7c8ccccc8c8c(-c9ccccc9)cccc87)cccc56)n4)ccc23)cc1 ; c1ccc(-c2nc(-c3ccc4c(c3)oc3c(-c5ccccc5)cccc34)nc(-c3ccc4c(c3)oc3c(-n5c6ccccc6c6cccc(-c7ccccc7)c65)cccc34)n2)cc1]

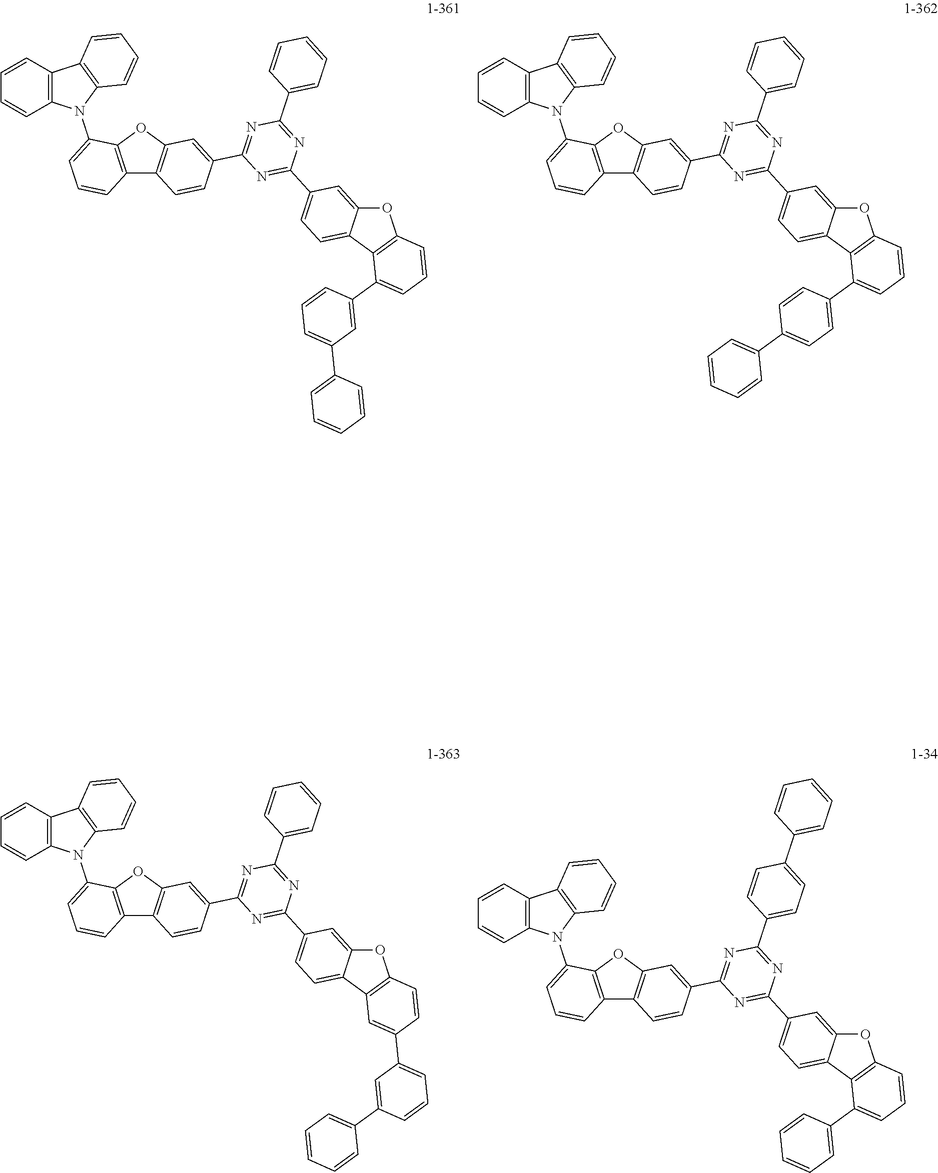 [SMILES: c1ccc(-c2cccc(-c3cccc4oc5cc(-c6nc(-c7ccccc7)nc(-c7ccc8c(c7)oc7c(-n9c%10ccccc%10c%10ccccc%109)cccc78)n6)ccc5c34)c2)cc1 ; c1ccc(-c2ccc(-c3cccc4oc5cc(-c6nc(-c7ccccc7)nc(-c7ccc8c(c7)oc7c(-n9c%10ccccc%10c%10ccccc%109)cccc78)n6)ccc5c34)cc2)cc1 ; c1ccc(-c2cccc(-c3ccc4oc5cc(-c6nc(-c7ccccc7)nc(-c7ccc8c(c7)oc7c(-n9c%10ccccc%10c%10ccccc%109)cccc78)n6)ccc5c4c3)c2)cc1 ; c1ccc(-c2ccc(-c3nc(-c4ccc5c(c4)oc4c(-n6c7ccccc7c7ccccc76)cccc45)nc(-c4ccc5c(c4)oc4cccc(-c6ccccc6)c45)n3)cc2)cc1]

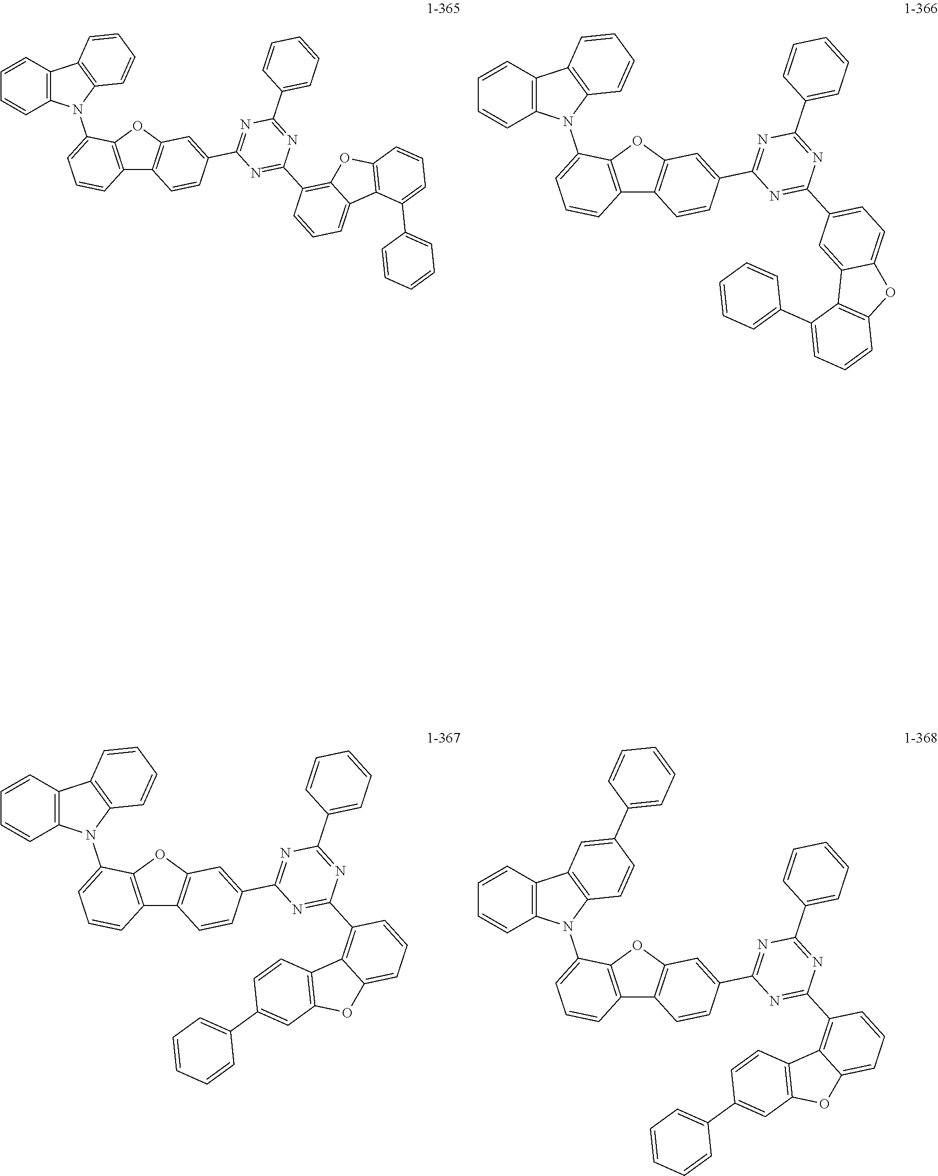 [SMILES: c1ccc(-c2nc(-c3ccc4c(c3)oc3c(-n5c6ccccc6c6ccccc65)cccc34)nc(-c3cccc4c3oc3cccc(-c5ccccc5)c34)n2)cc1 ; c1ccc(-c2nc(-c3ccc4c(c3)oc3c(-n5c6ccccc6c6ccccc65)cccc34)nc(-c3ccc4oc5cccc(-c6ccccc6)c5c4c3)n2)cc1 ; c1ccc(-c2ccc3c(c2)oc2cccc(-c4nc(-c5ccccc5)nc(-c5ccc6c(c5)oc5c(-n7c8ccccc8c8ccccc87)cccc56)n4)c23)cc1 ; c1ccc(-c2ccc3c(c2)oc2cccc(-c4nc(-c5ccccc5)nc(-c5ccc6c(c5)oc5c(-n7c8ccccc8c8cc(-c9ccccc9)ccc87)cccc56)n4)c23)cc1]

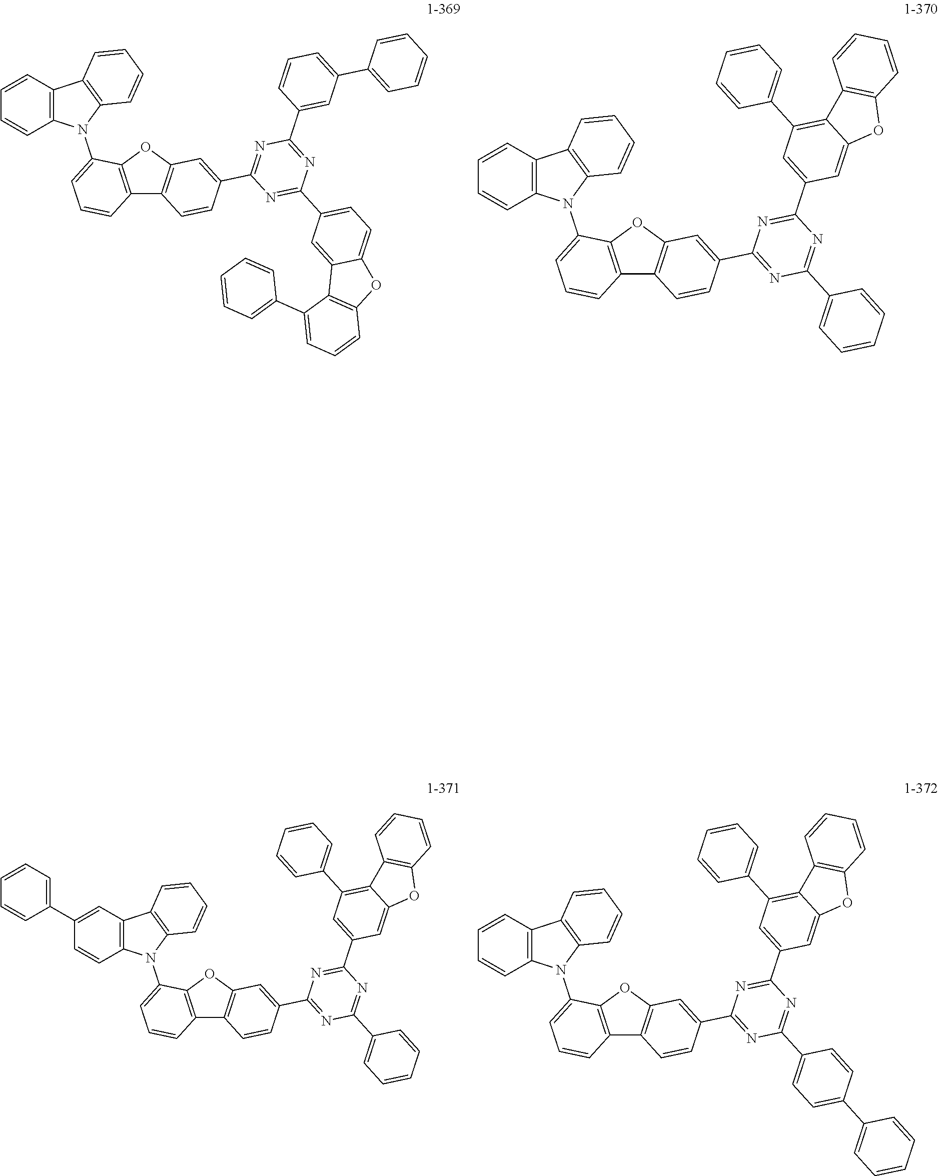 [SMILES: c1ccc(-c2cccc(-c3nc(-c4ccc5c(c4)oc4c(-n6c7ccccc7c7ccccc76)cccc45)nc(-c4ccc5oc6cccc(-c7ccccc7)c6c5c4)n3)c2)cc1 ; c1ccc(-c2nc(-c3ccc4c(c3)oc3c(-n5c6ccccc6c6ccccc65)cccc34)nc(-c3cc(-c4ccccc4)c4c(c3)oc3ccccc34)n2)cc1 ; c1ccc(-c2ccc3c(c2)c2ccccc2n3-c2cccc3c2oc2cc(-c4nc(-c5ccccc5)nc(-c5cc(-c6ccccc6)c6c(c5)oc5ccccc56)n4)ccc23)cc1 ; c1ccc(-c2ccc(-c3nc(-c4ccc5c(c4)oc4c(-n6c7ccccc7c7ccccc76)cccc45)nc(-c4cc(-c5ccccc5)c5c(c4)oc4ccccc45)n3)cc2)cc1]

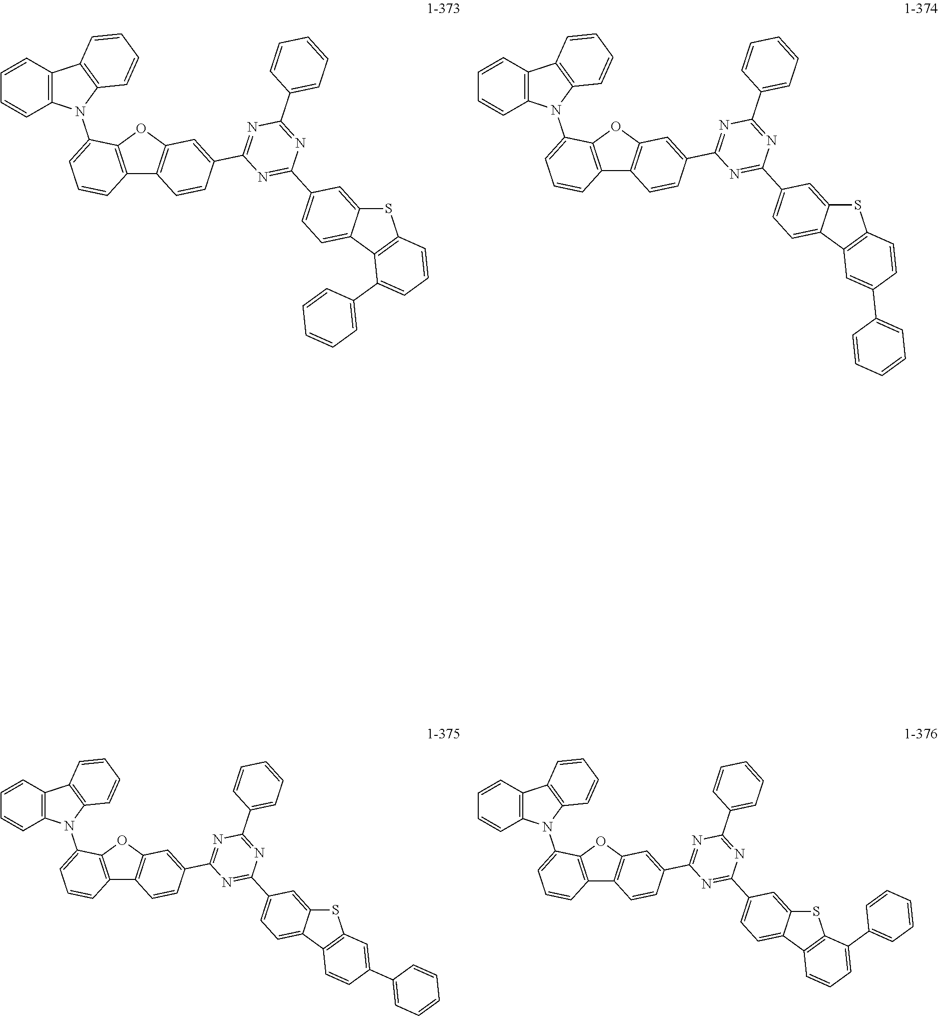 [SMILES: c1ccc(-c2nc(-c3ccc4c(c3)oc3c(-n5c6ccccc6c6ccccc65)cccc34)nc(-c3ccc4c(c3)sc3cccc(-c5ccccc5)c34)n2)cc1 ; c1ccc(-c2ccc3sc4cc(-c5nc(-c6ccccc6)nc(-c6ccc7c(c6)oc6c(-n8c9ccccc9c9ccccc98)cccc67)n5)ccc4c3c2)cc1 ; c1ccc(-c2ccc3c(c2)sc2cc(-c4nc(-c5ccccc5)nc(-c5ccc6c(c5)oc5c(-n7c8ccccc8c8ccccc87)cccc56)n4)ccc23)cc1 ; c1ccc(-c2nc(-c3ccc4c(c3)oc3c(-n5c6ccccc6c6ccccc65)cccc34)nc(-c3ccc4c(c3)sc3c(-c5ccccc5)cccc34)n2)cc1]

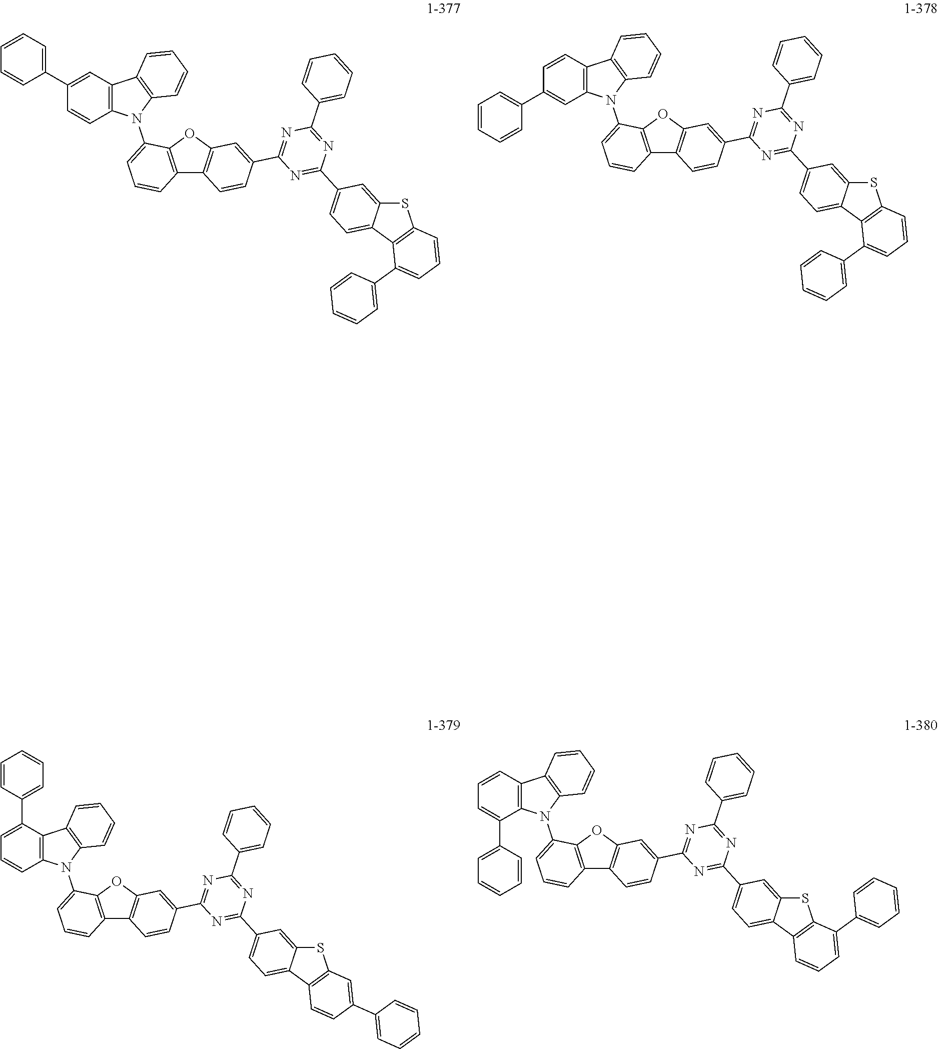 [SMILES: c1ccc(-c2ccc3c(c2)c2ccccc2n3-c2cccc3c2oc2cc(-c4nc(-c5ccccc5)nc(-c5ccc6c(c5)sc5cccc(-c7ccccc7)c56)n4)ccc23)cc1 ; c1ccc(-c2ccc3c4ccccc4n(-c4cccc5c4oc4cc(-c6nc(-c7ccccc7)nc(-c7ccc8c(c7)sc7cccc(-c9ccccc9)c78)n6)ccc45)c3c2)cc1 ; c1ccc(-c2ccc3c(c2)sc2cc(-c4nc(-c5ccccc5)nc(-c5ccc6c(c5)oc5c(-n7c8ccccc8c8c(-c9ccccc9)cccc87)cccc56)n4)ccc23)cc1 ; c1ccc(-c2nc(-c3ccc4c(c3)oc3c(-n5c6ccccc6c6cccc(-c7ccccc7)c65)cccc34)nc(-c3ccc4c(c3)sc3c(-c5ccccc5)cccc34)n2)cc1]

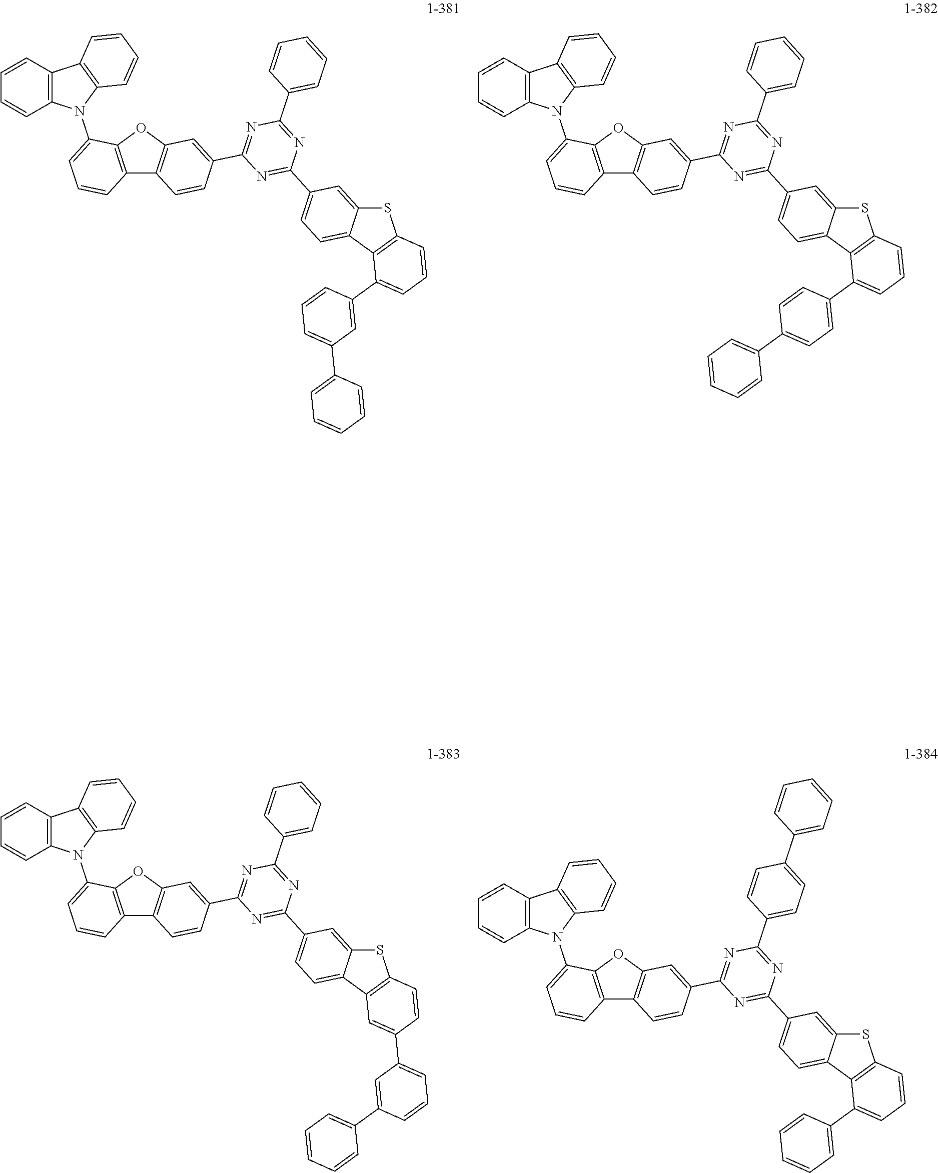 [SMILES: c1ccc(-c2cccc(-c3cccc4sc5cc(-c6nc(-c7ccccc7)nc(-c7ccc8c(c7)oc7c(-n9c%10ccccc%10c%10ccccc%109)cccc78)n6)ccc5c34)c2)cc1 ; c1ccc(-c2ccc(-c3cccc4sc5cc(-c6nc(-c7ccccc7)nc(-c7ccc8c(c7)oc7c(-n9c%10ccccc%10c%10ccccc%109)cccc78)n6)ccc5c34)cc2)cc1 ; c1ccc(-c2cccc(-c3ccc4sc5cc(-c6nc(-c7ccccc7)nc(-c7ccc8c(c7)oc7c(-n9c%10ccccc%10c%10ccccc%109)cccc78)n6)ccc5c4c3)c2)cc1 ; c1ccc(-c2ccc(-c3nc(-c4ccc5c(c4)oc4c(-n6c7ccccc7c7ccccc76)cccc45)nc(-c4ccc5c(c4)sc4cccc(-c6ccccc6)c45)n3)cc2)cc1]

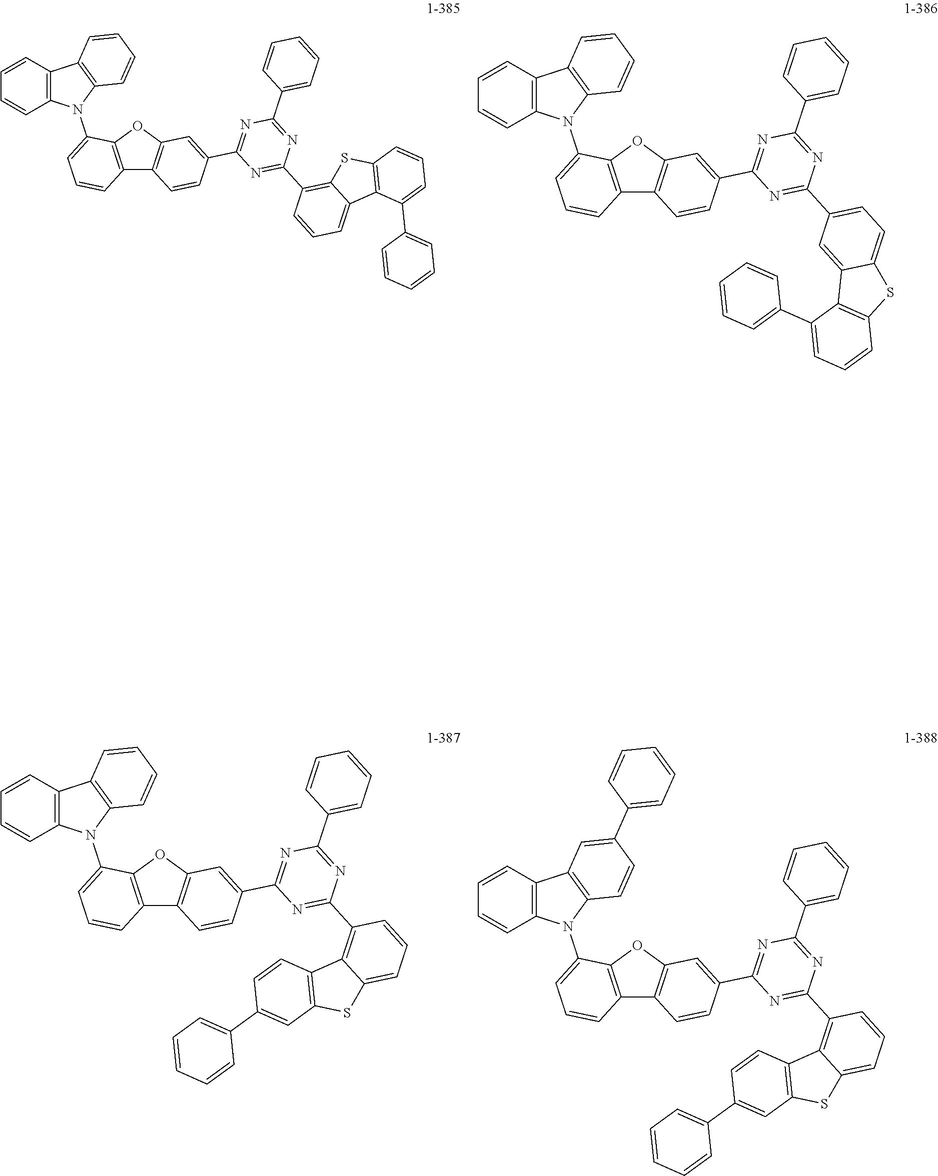 [SMILES: c1ccc(-c2nc(-c3ccc4c(c3)oc3c(-n5c6ccccc6c6ccccc65)cccc34)nc(-c3cccc4c3sc3cccc(-c5ccccc5)c34)n2)cc1 ; c1ccc(-c2nc(-c3ccc4c(c3)oc3c(-n5c6ccccc6c6ccccc65)cccc34)nc(-c3ccc4sc5cccc(-c6ccccc6)c5c4c3)n2)cc1 ; c1ccc(-c2ccc3c(c2)sc2cccc(-c4nc(-c5ccccc5)nc(-c5ccc6c(c5)oc5c(-n7c8ccccc8c8ccccc87)cccc56)n4)c23)cc1 ; c1ccc(-c2ccc3c(c2)sc2cccc(-c4nc(-c5ccccc5)nc(-c5ccc6c(c5)oc5c(-n7c8ccccc8c8cc(-c9ccccc9)ccc87)cccc56)n4)c23)cc1]

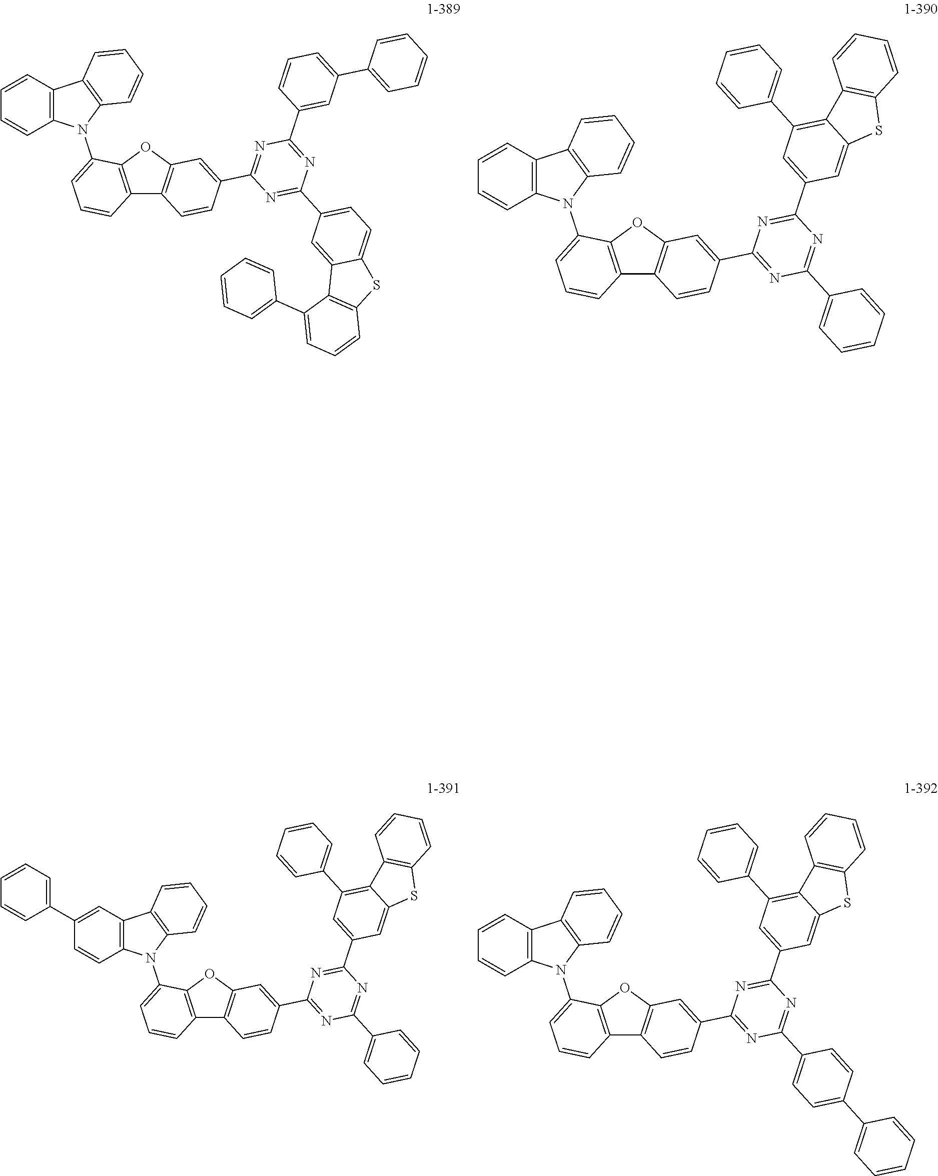 [SMILES: c1ccc(-c2cccc(-c3nc(-c4ccc5c(c4)oc4c(-n6c7ccccc7c7ccccc76)cccc45)nc(-c4ccc5sc6cccc(-c7ccccc7)c6c5c4)n3)c2)cc1 ; c1ccc(-c2nc(-c3ccc4c(c3)oc3c(-n5c6ccccc6c6ccccc65)cccc34)nc(-c3cc(-c4ccccc4)c4c(c3)sc3ccccc34)n2)cc1 ; c1ccc(-c2ccc3c(c2)c2ccccc2n3-c2cccc3c2oc2cc(-c4nc(-c5ccccc5)nc(-c5cc(-c6ccccc6)c6c(c5)sc5ccccc56)n4)ccc23)cc1 ; c1ccc(-c2ccc(-c3nc(-c4ccc5c(c4)oc4c(-n6c7ccccc7c7ccccc76)cccc45)nc(-c4cc(-c5ccccc5)c5c(c4)sc4ccccc45)n3)cc2)cc1]

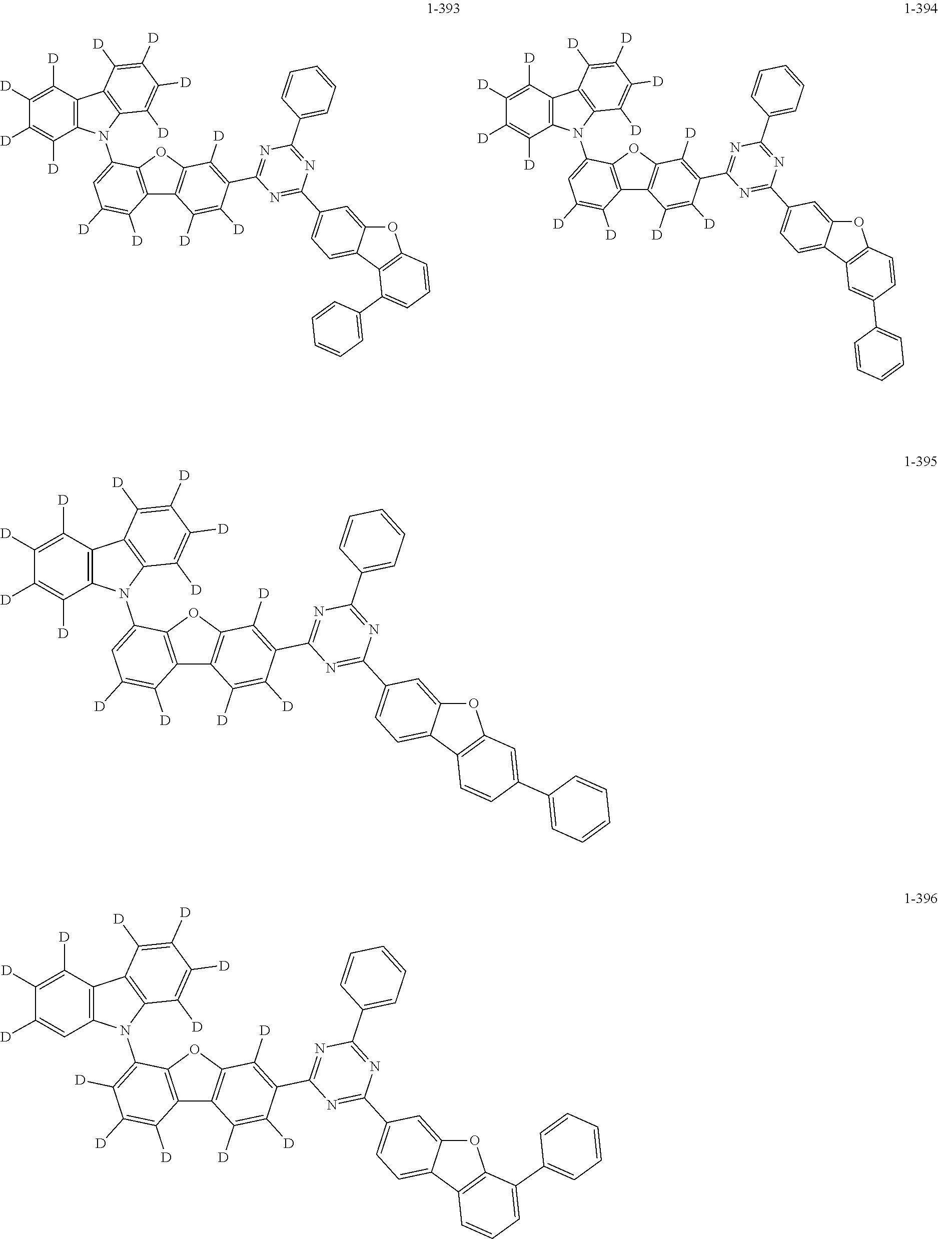

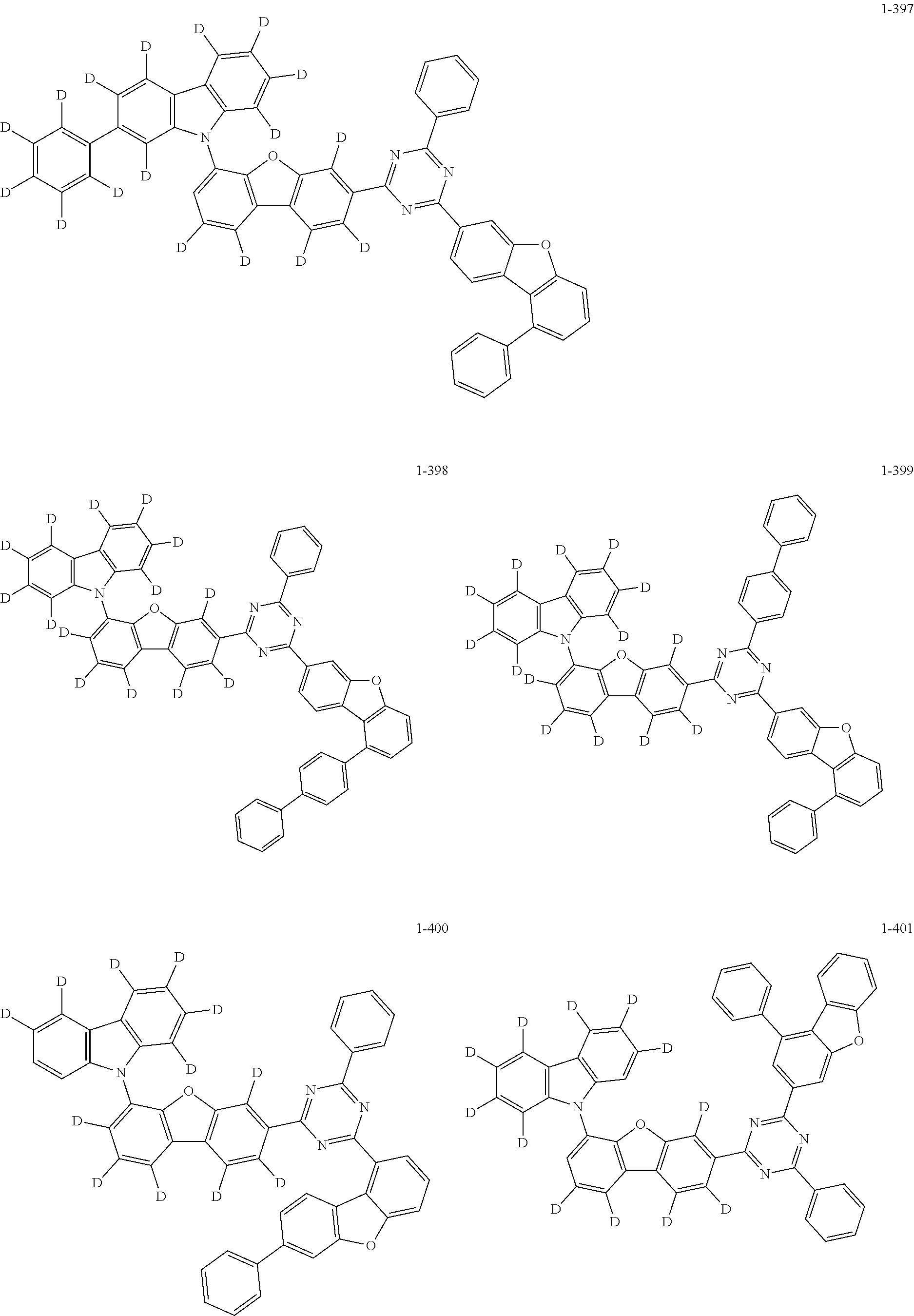

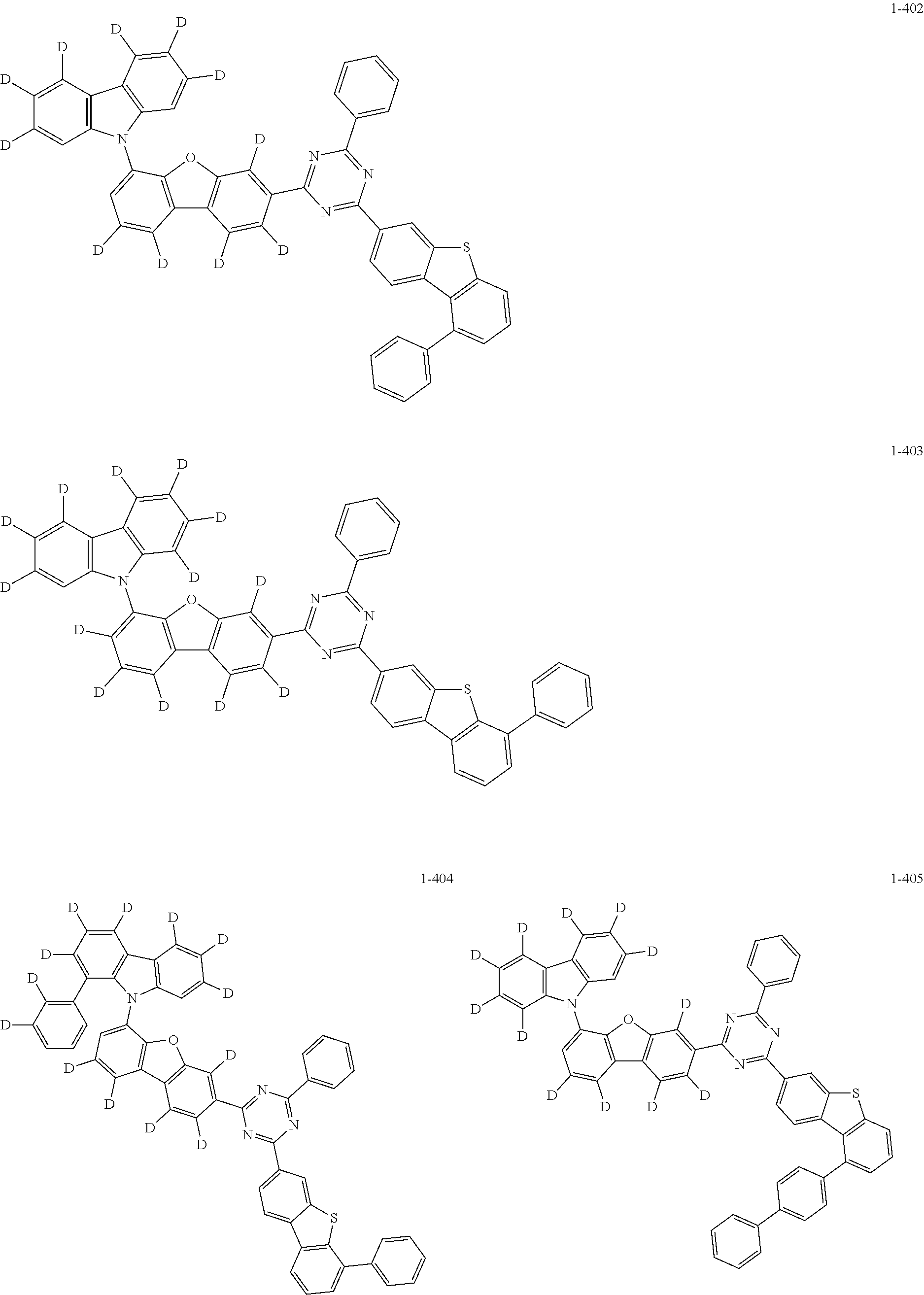

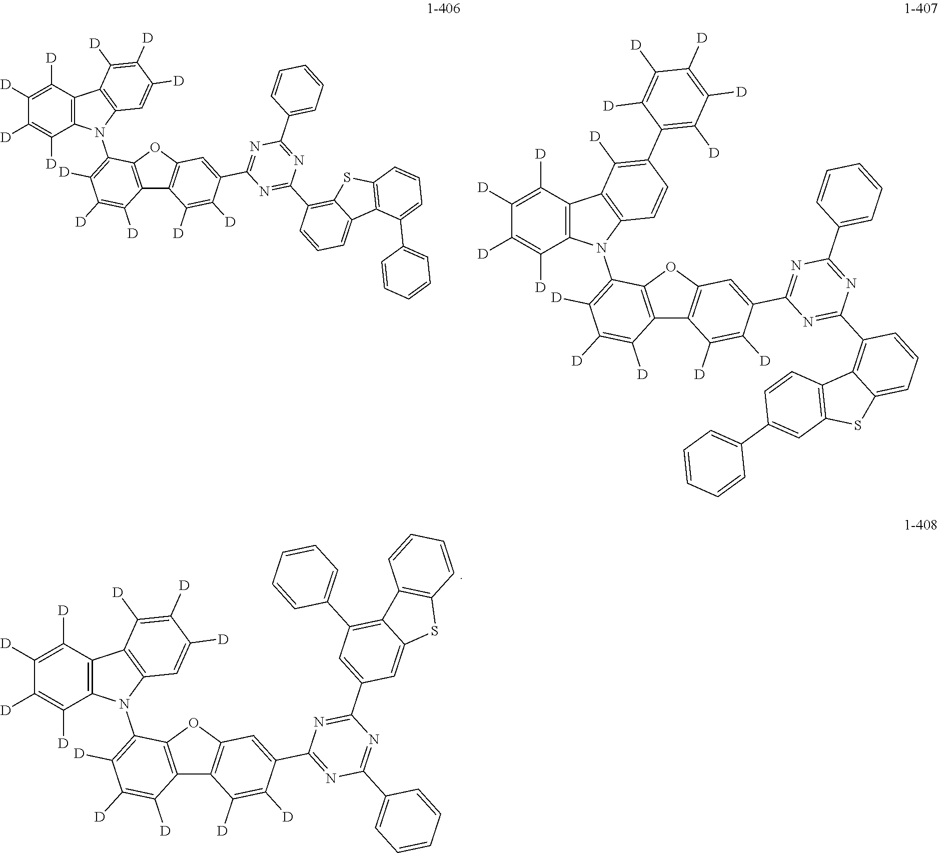

6. An organic light emitting device comprising:
a first electrode;
a second electrode provided opposite to the first electrode; and
one or more organic material layers provided between the first electrode and the second electrode,
wherein one or more layers of the organic material layers include the heterocyclic compound of claim 1.

7. The organic light emitting device of claim 6, wherein the organic material layer includes a light emitting layer, and the light emitting layer includes the heterocyclic compound represented by Chemical Formula 1.

8. The organic light emitting device of claim 6, wherein the organic material layer includes a light emitting layer, the light emitting layer includes a host material, and the host material includes the heterocyclic compound represented by Chemical Formula 1.

9. The organic light emitting device of claim 6, wherein the organic material layer further includes a heterocyclic compound represented by the following Chemical Formula 2 or Chemical Formula 3: 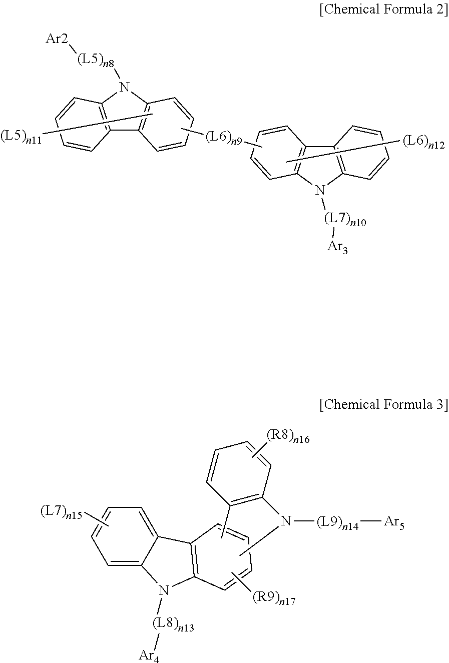
in Chemical Formulae 2 and 3,
L5 to L9 are the same as or different from each other, and each independently a single bond; a substituted or unsubstituted C6 to C60 arylene group; or a substituted or unsubstituted C2 to C60 heteroarylene group;
Ar2 to Ar5 are the same as or different from each other, and each independently a substituted or unsubstituted C6 to C60 aryl group; or a substituted or unsubstituted C2 to C60 heteroaryl group;
R5 to R9 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102 and —SiR101R102R103, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group;
n8 to n10, n13 and n14 are the same as or different from each other, and each independently an integer of 0 to 3;
n11 and n12 are an integer of 0 to 7;
n15 and n16 are an integer of 0 to 4; and
n17 is an integer of 0 to 2.

10. The organic light emitting device of claim 9, wherein Ar2 to Ar5 are represented by any one of the following Structural Formula 3 or 4: 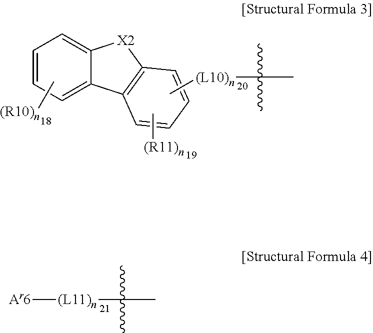
in Structural Formulae 3 and 4,
X2 is O, S or CRaRb;
L10 and L11 are the same as or different from each other, and each independently a single bond; a substituted or unsubstituted C6 to C60 arylene group; or a substituted or unsubstituted C2 to C60 heteroarylene group;
Ar6 is a substituted or unsubstituted C6 to C60 aryl group;
R10, R11, Ra and Rb are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102 and —SiR101R102R103, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group;
n18 is an integer of 0 to 4;
n19 is an integer of 0 to 3; and
n20 and n21 are the same as or different from each other, and each independently an integer of 0 to 3.

11. The organic light emitting device of claim 9, wherein the heterocyclic compound represented by Chemical Formula 2 or 3 does not include deuterium as a substituent, or has a deuterium content of 1% to 100% based on a total number of hydrogen atoms and deuterium atoms.

12. The organic light emitting device of claim 9, wherein the heterocyclic compound represented by Chemical Formula 2 is represented by any one of the following compounds:

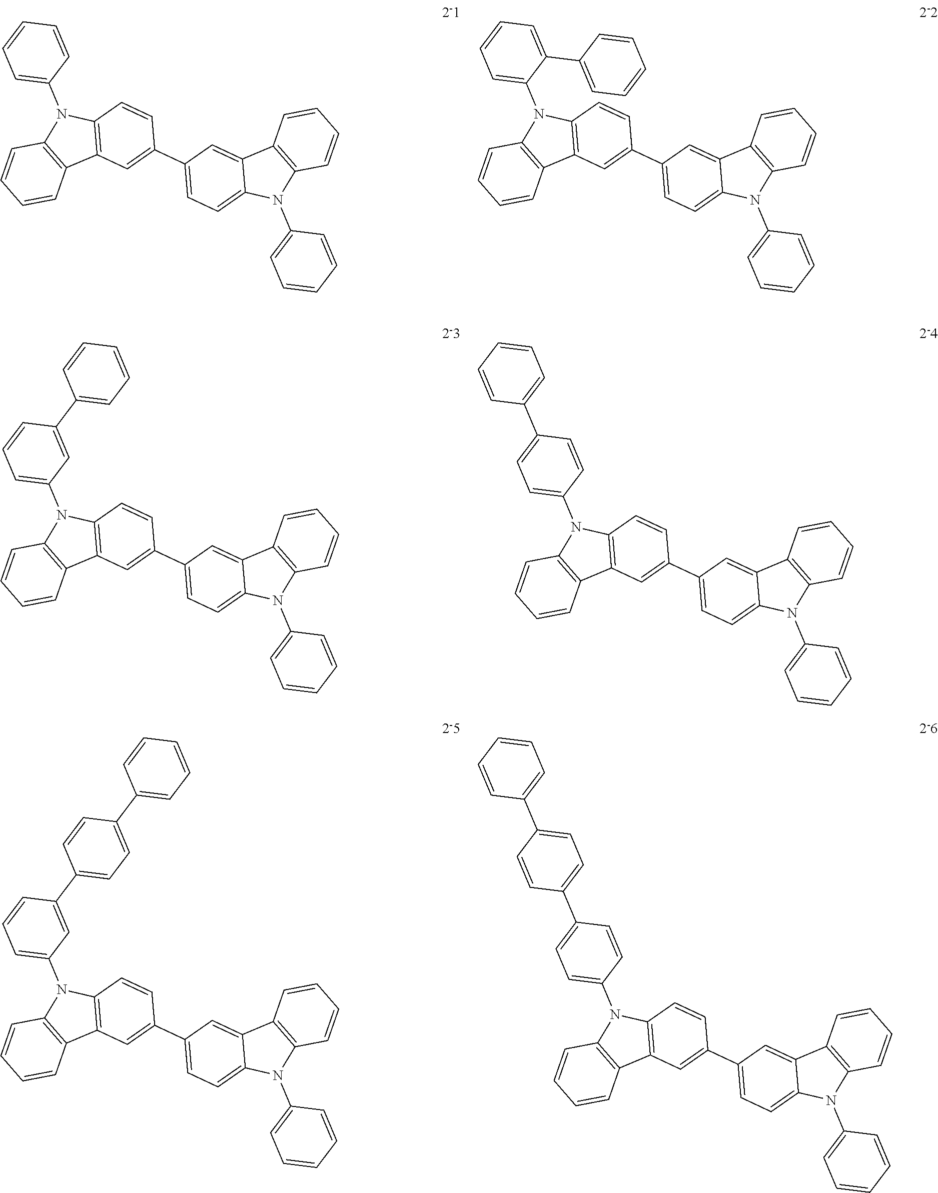 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

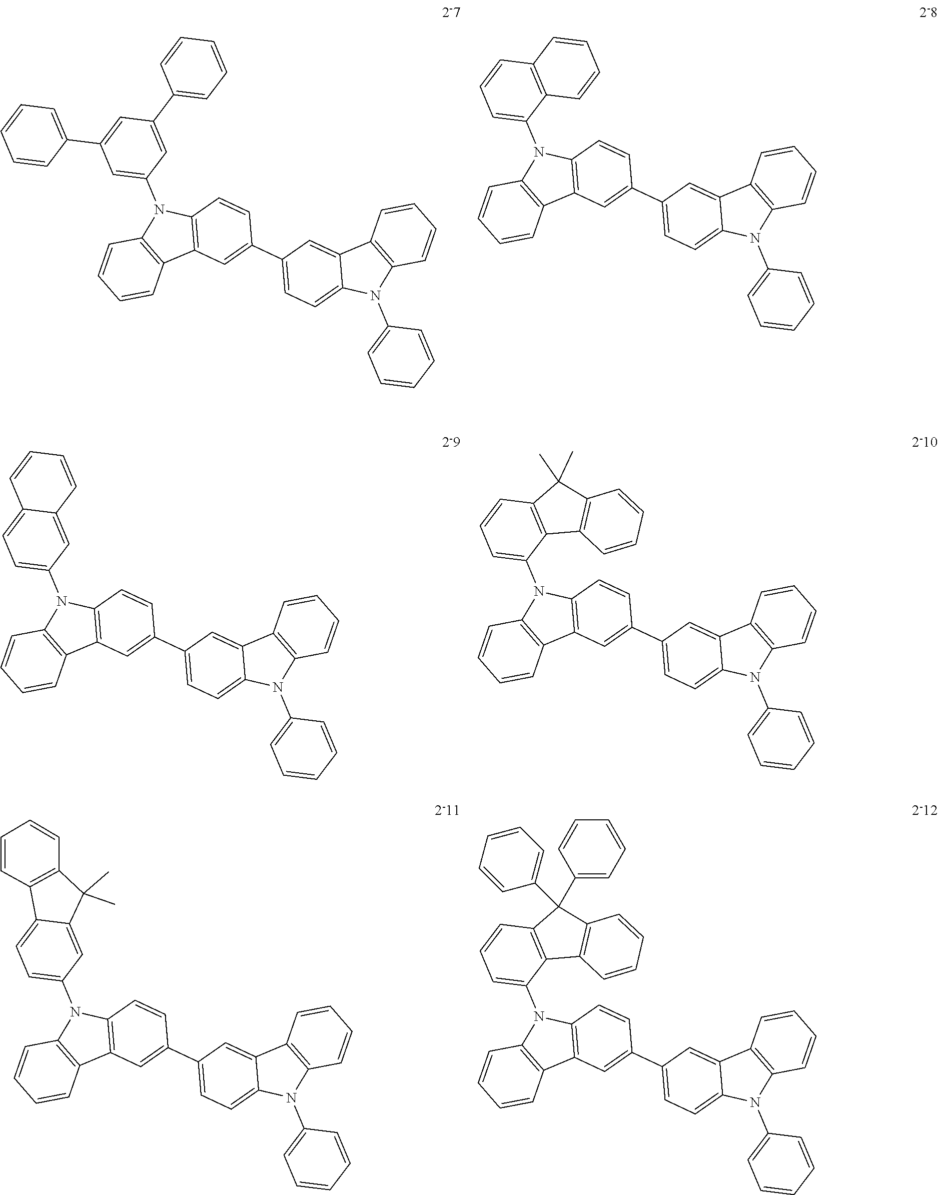

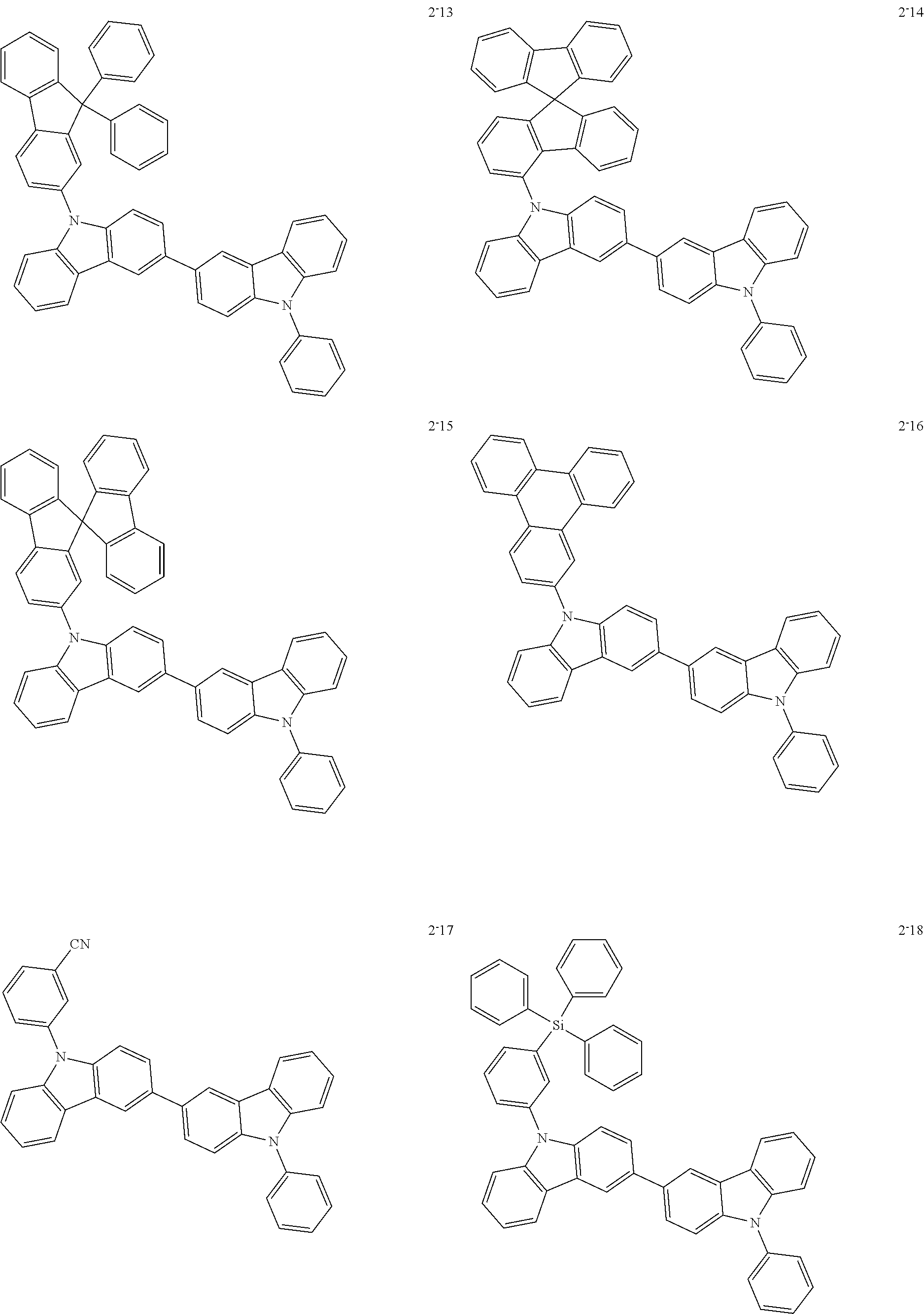

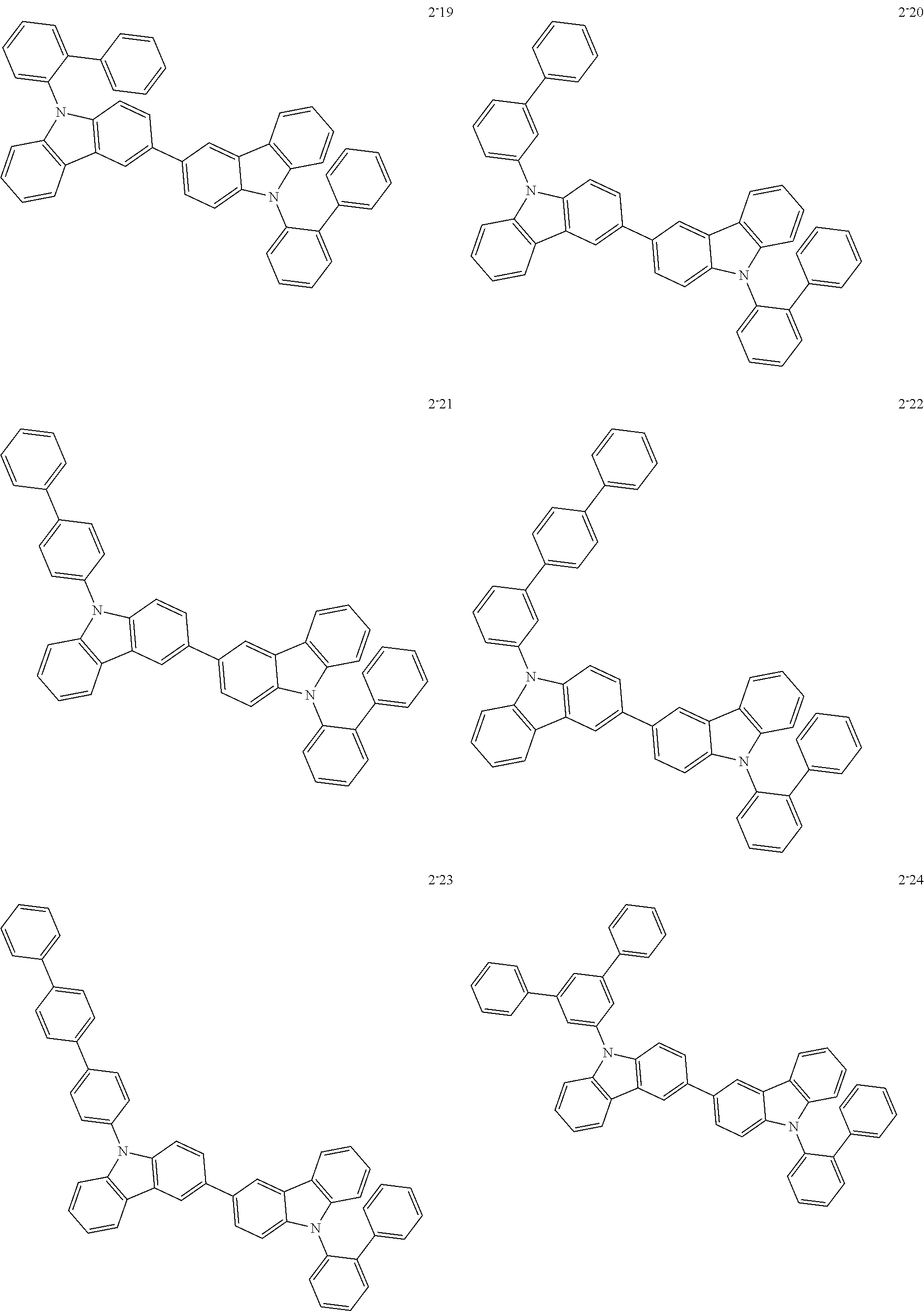

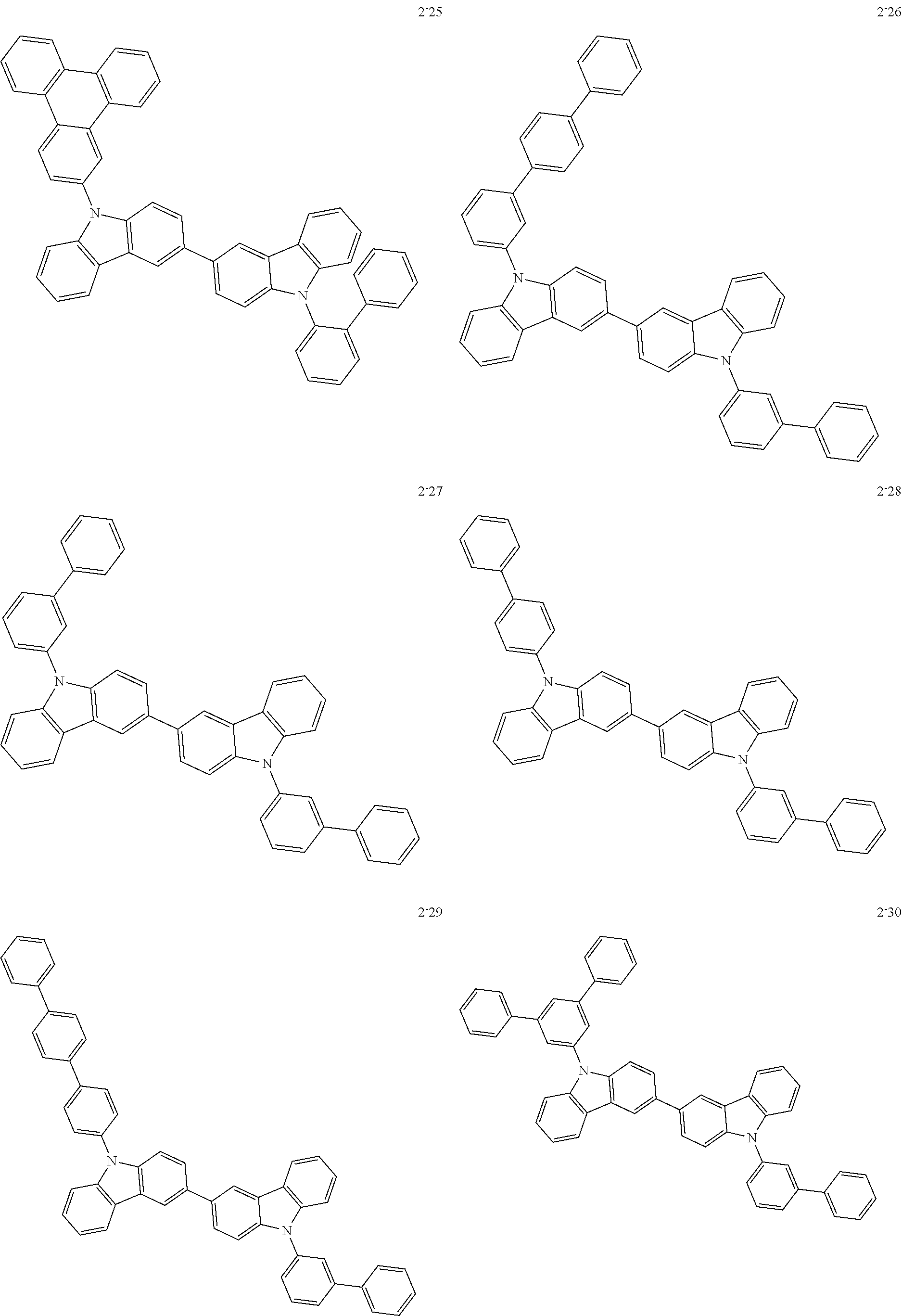

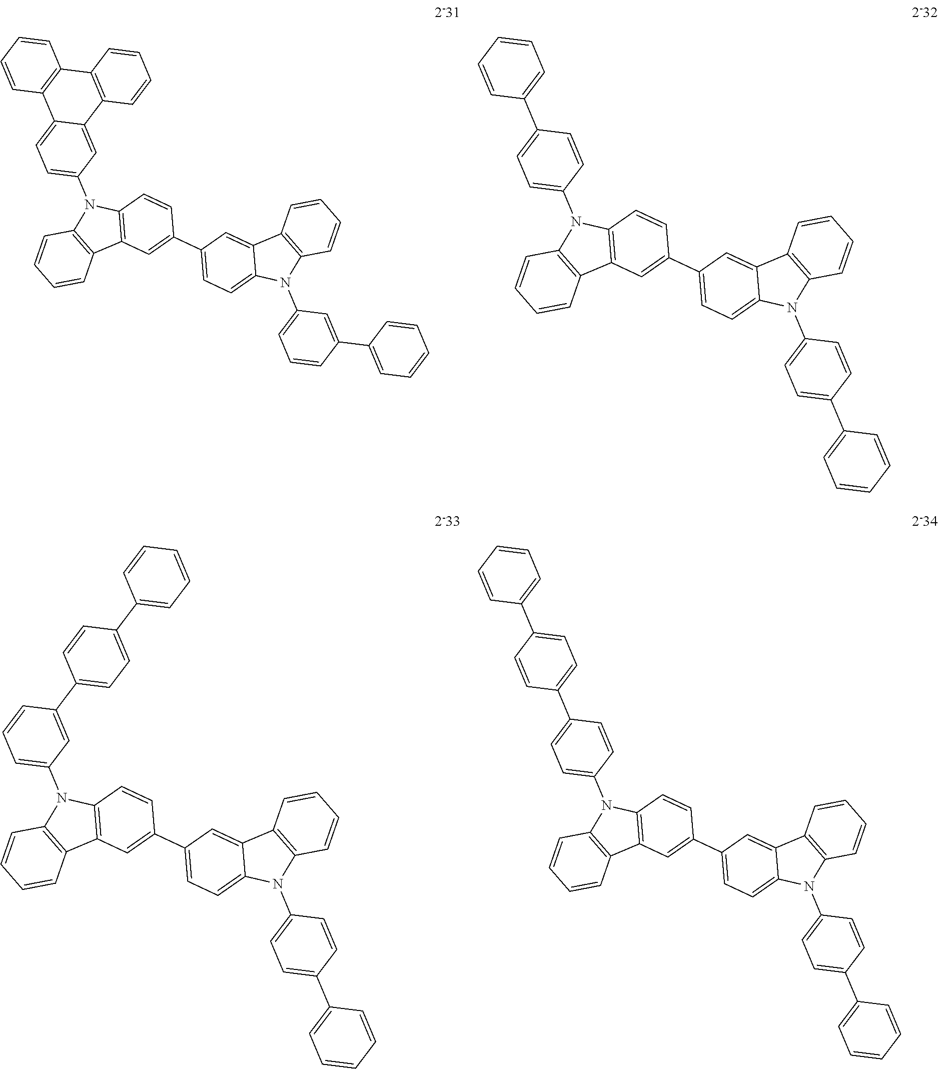 [SMILES: c1ccc(-c2cccc(-n3c4ccccc4c4cc(-c5ccc6c(c5)c5ccccc5n6-c5ccc6c7ccccc7c7ccccc7c6c5)ccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1 ; c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1]

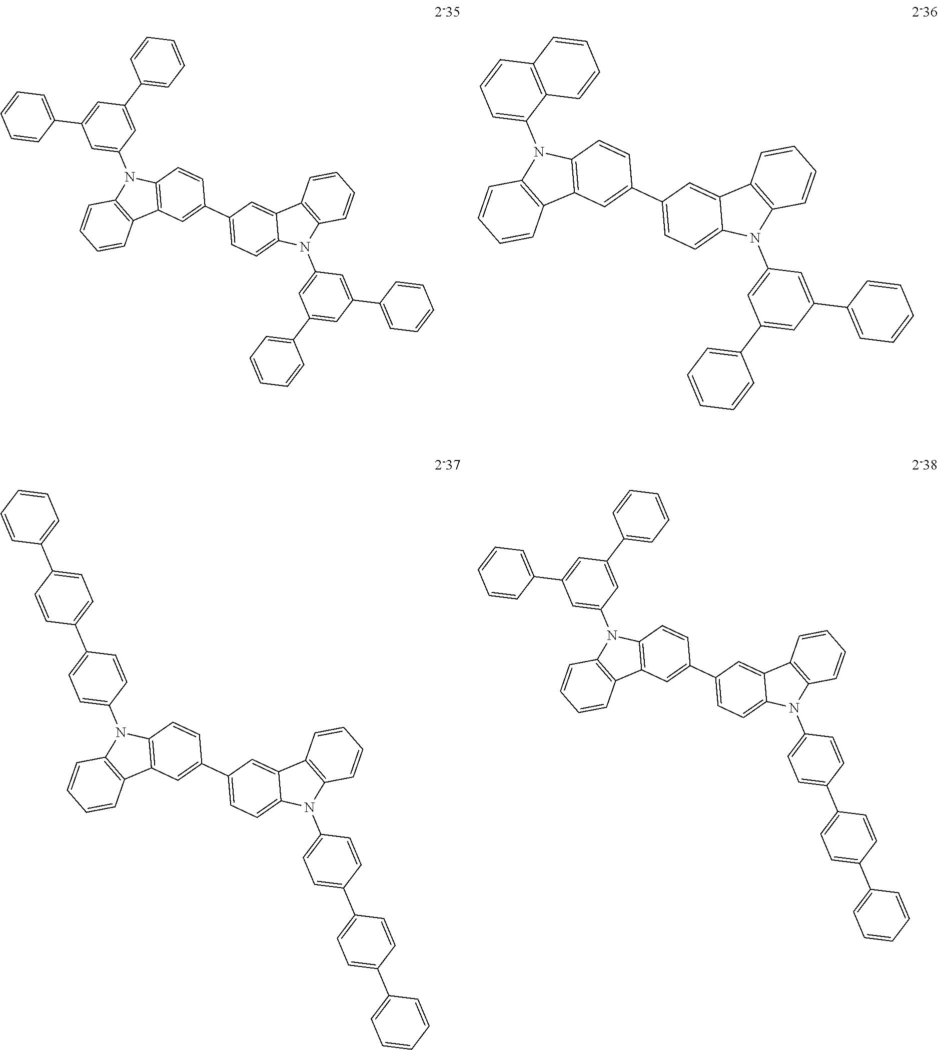 [SMILES: c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cc(-c3ccccc3)cc(-n3c4ccccc4c4cc(-c5ccc6c(c5)c5ccccc5n6-c5cccc6ccccc56)ccc43)c2)cc1 ; c1ccc(-c2ccc(-c3ccc(-n4c5ccccc5c5cc(-c6ccc7c(c6)c6ccccc6n7-c6ccc(-c7ccc(-c8ccccc8)cc7)cc6)ccc54)cc3)cc2)cc1 ; c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1]

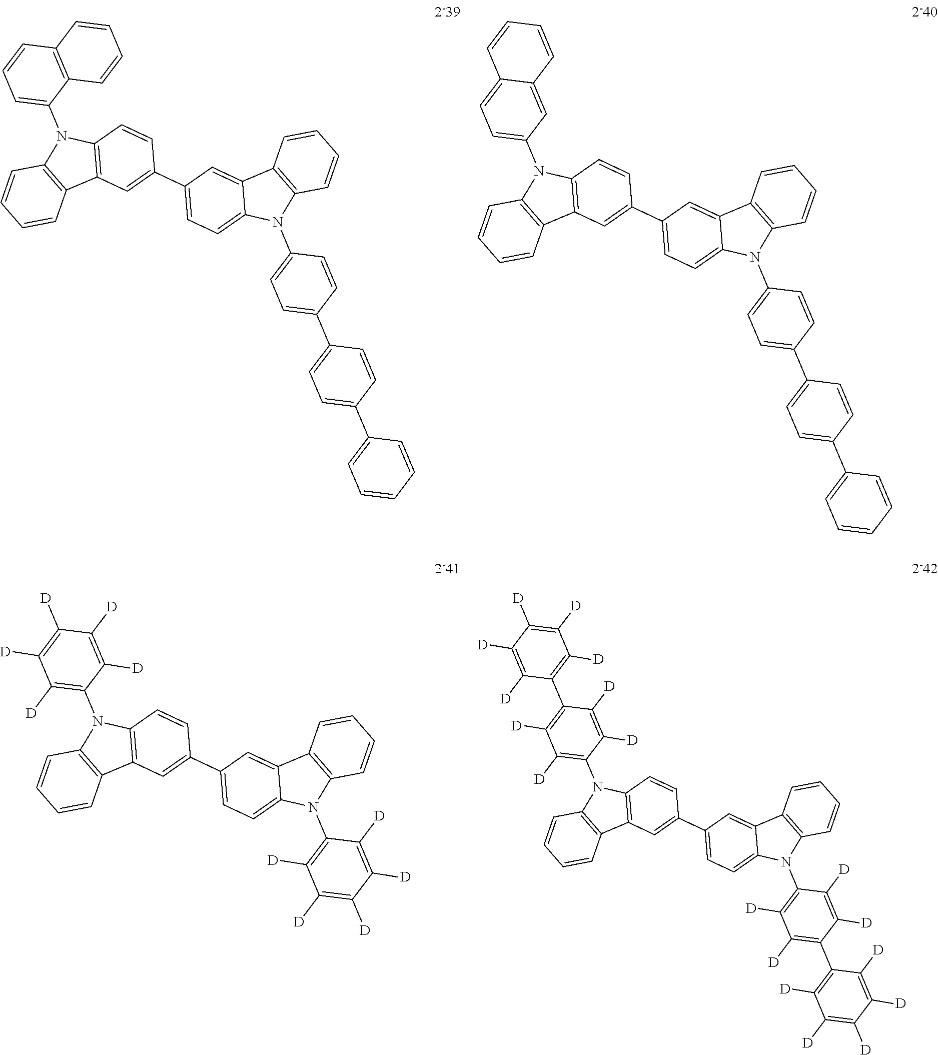 [SMILES: c1ccc(-c2ccc(-c3ccc(-n4c5ccccc5c5cc(-c6ccc7c(c6)c6ccccc6n7-c6cccc7ccccc67)ccc54)cc3)cc2)cc1 ; c1ccc(-c2ccc(-c3ccc(-n4c5ccccc5c5cc(-c6ccc7c(c6)c6ccccc6n7-c6ccc7ccccc7c6)ccc54)cc3)cc2)cc1 ; [2H]c1c([2H])c([2H])c(-n2c3ccccc3c3cc(-c4ccc5c(c4)c4ccccc4n5-c4c([2H])c([2H])c([2H])c([2H])c4[2H])ccc32)c([2H])c1[2H] ; [2H]c1c([2H])c([2H])c(-c2c([2H])c([2H])c(-n3c4ccccc4c4cc(-c5ccc6c(c5)c5ccccc5n6-c5c([2H])c([2H])c(-c6c([2H])c([2H])c([2H])c([2H])c6[2H])c([2H])c5[2H])ccc43)c([2H])c2[2H])c([2H])c1[2H]]

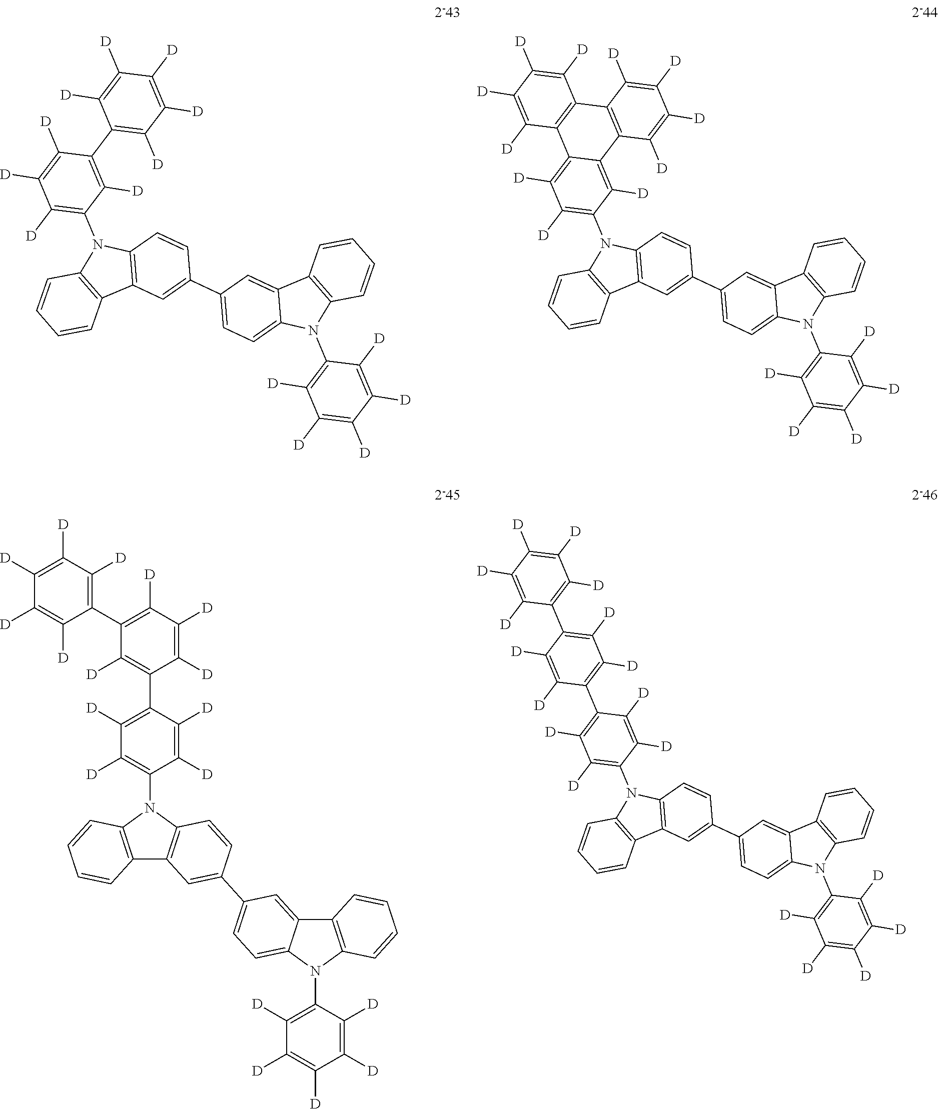

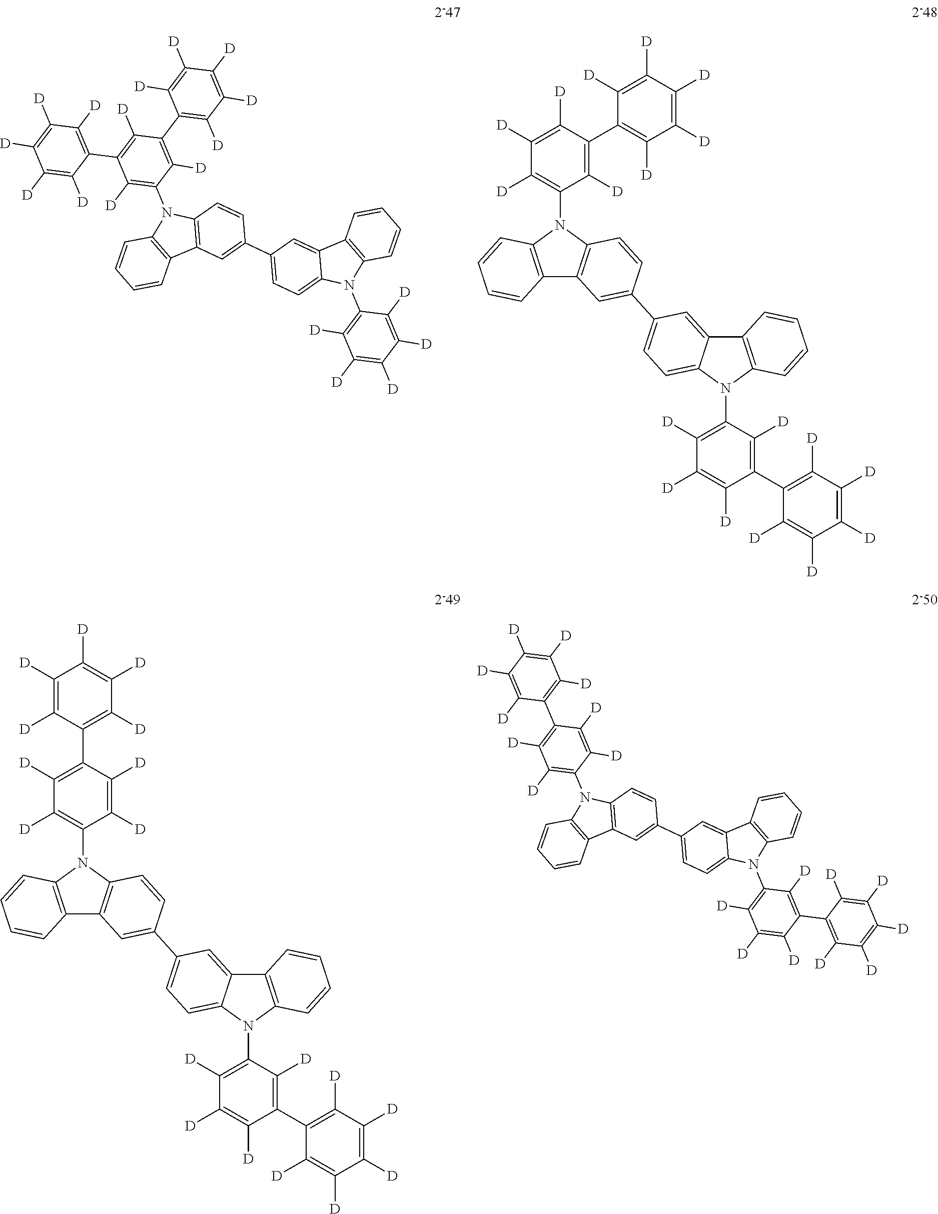

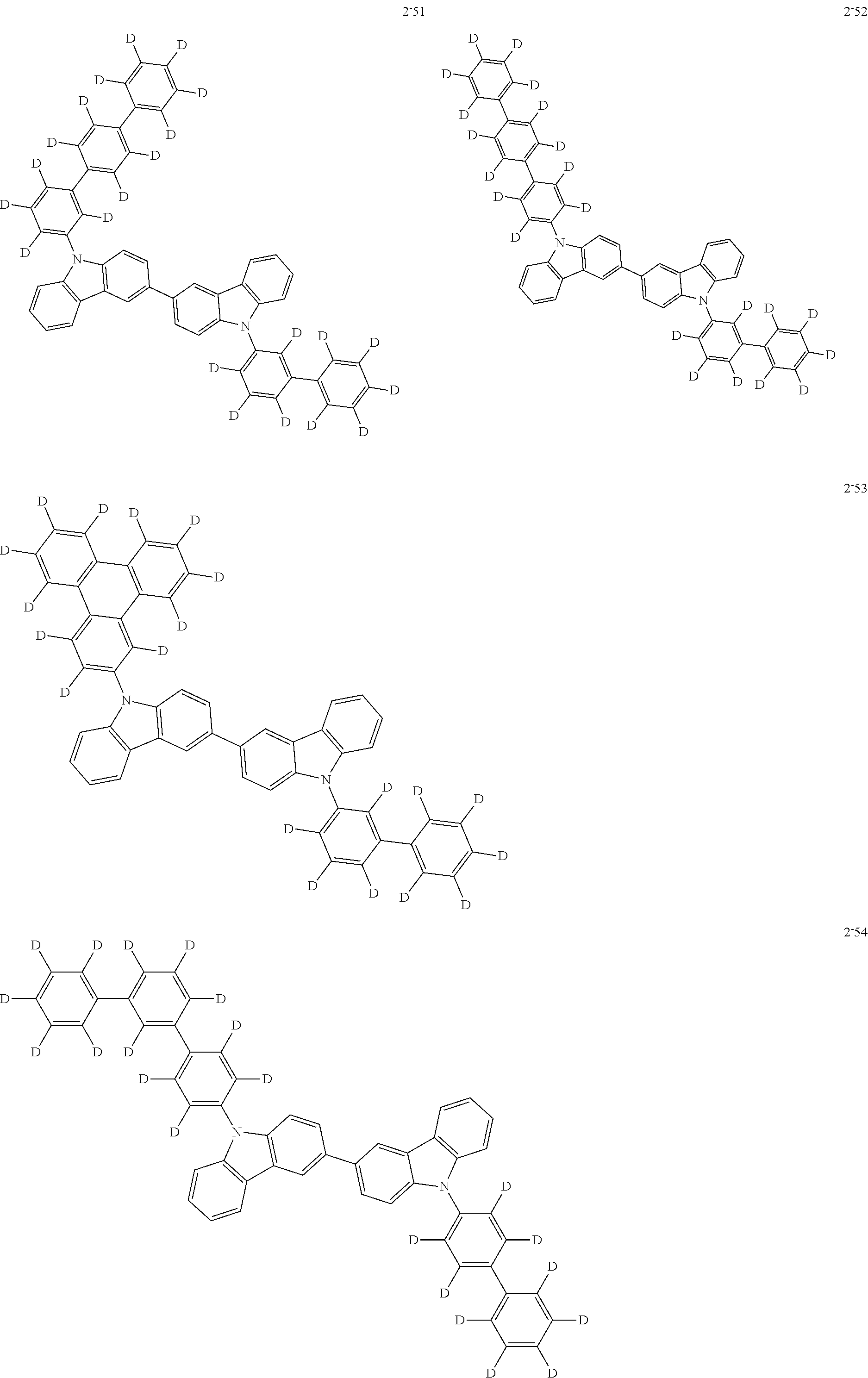

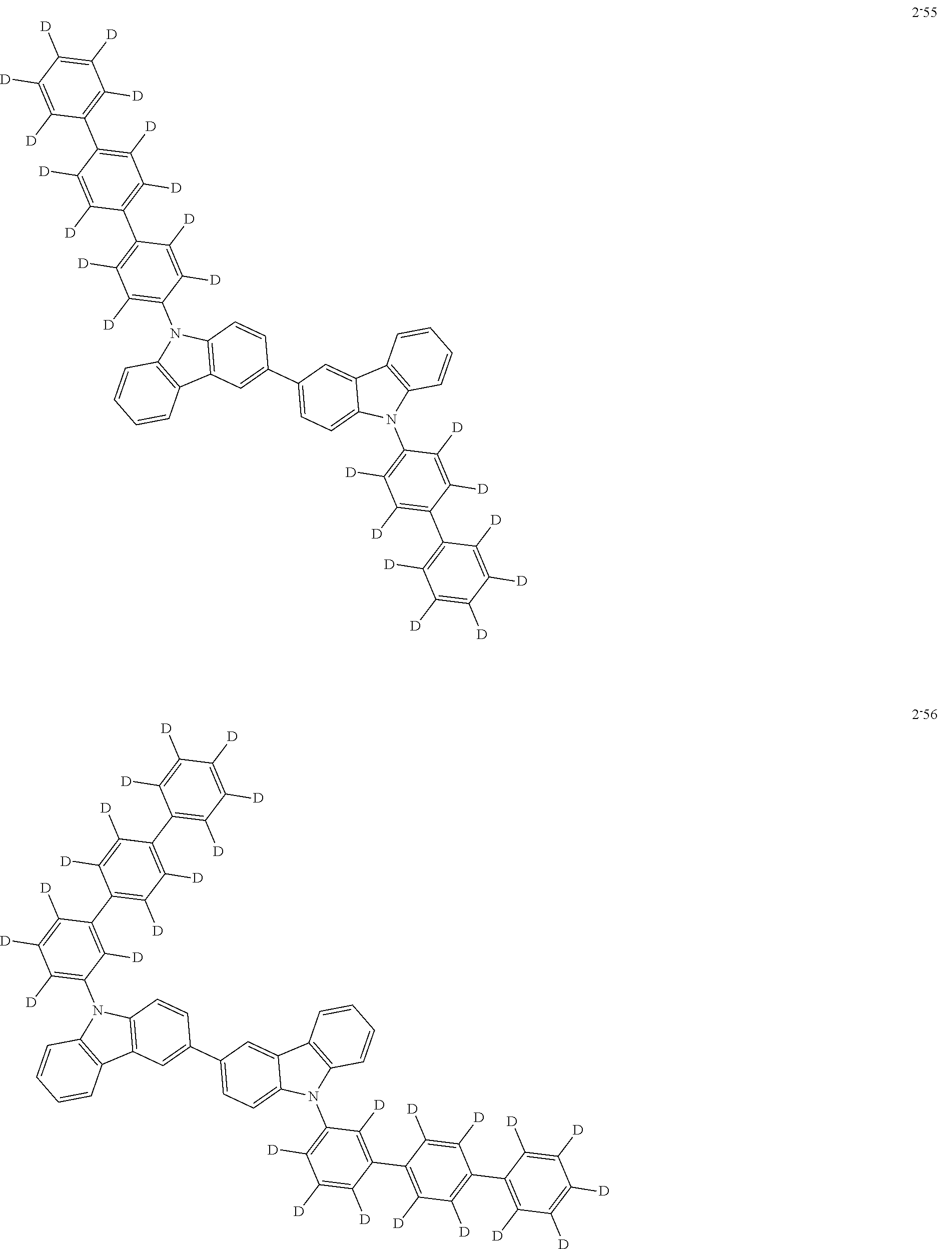 [SMILES: [2H]c1c([2H])c([2H])c(-c2c([2H])c([2H])c(-c3c([2H])c([2H])c(-n4c5ccccc5c5cc(-c6ccc7c(c6)c6ccccc6n7-c6c([2H])c([2H])c(-c7c([2H])c([2H])c([2H])c([2H])c7[2H])c([2H])c6[2H])ccc54)c([2H])c3[2H])c([2H])c2[2H])c([2H])c1[2H] ; [2H]c1c([2H])c([2H])c(-c2c([2H])c([2H])c(-c3c([2H])c([2H])c([2H])c(-n4c5ccccc5c5cc(-c6ccc7c(c6)c6ccccc6n7-c6c([2H])c([2H])c([2H])c(-c7c([2H])c([2H])c(-c8c([2H])c([2H])c([2H])c([2H])c8[2H])c([2H])c7[2H])c6[2H])ccc54)c3[2H])c([2H])c2[2H])c([2H])c1[2H]]

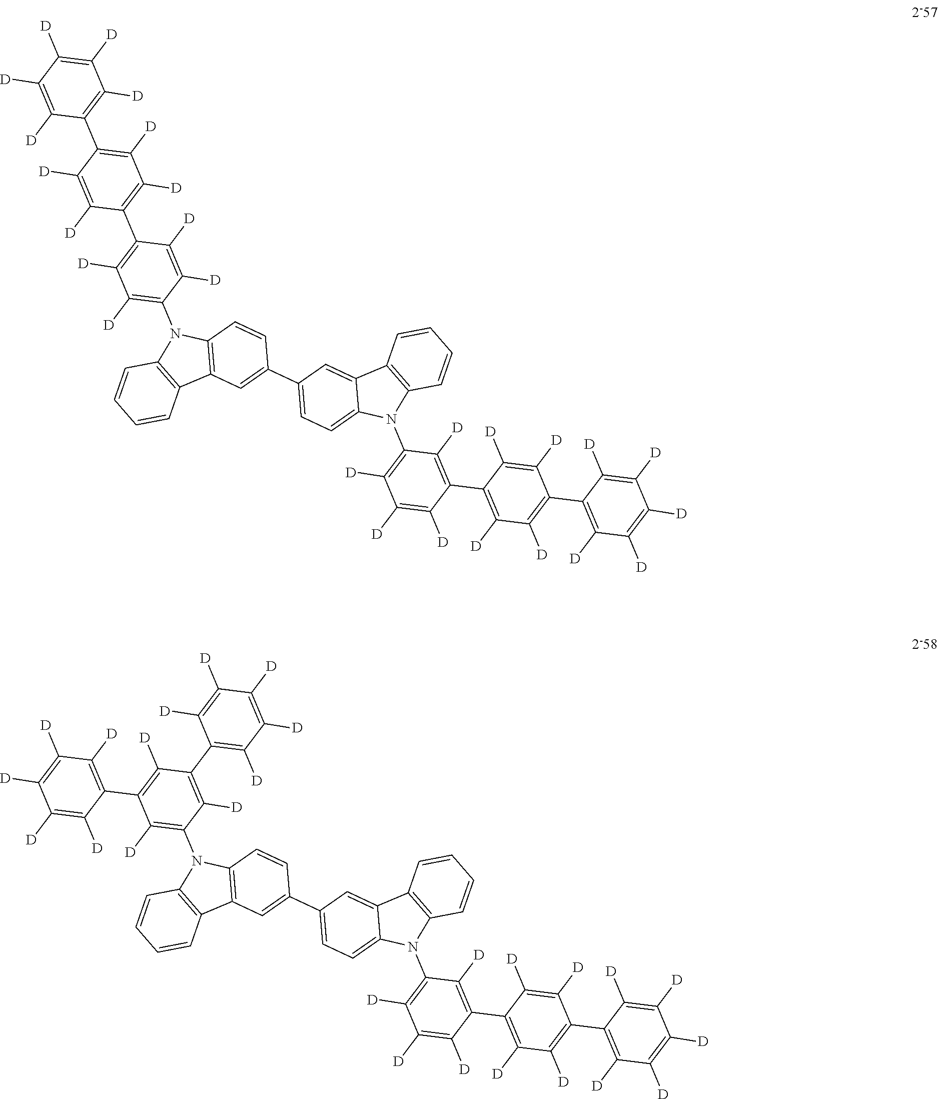

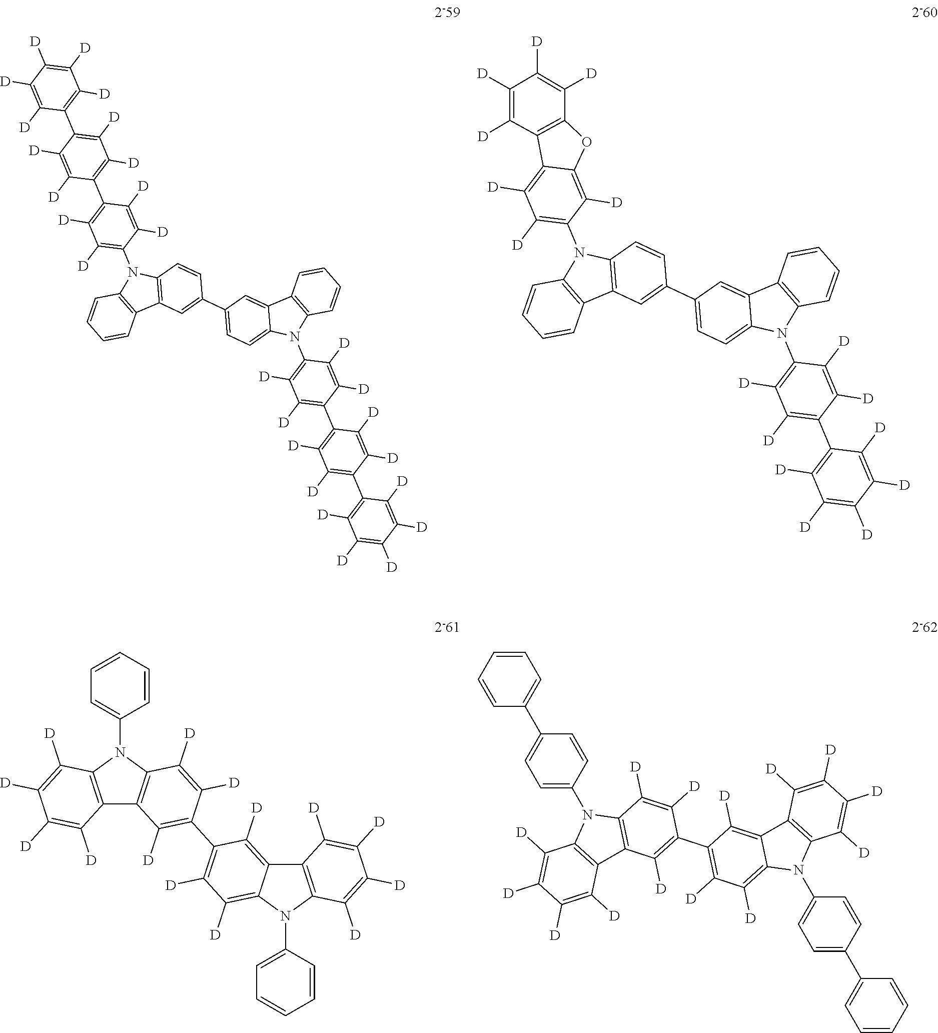

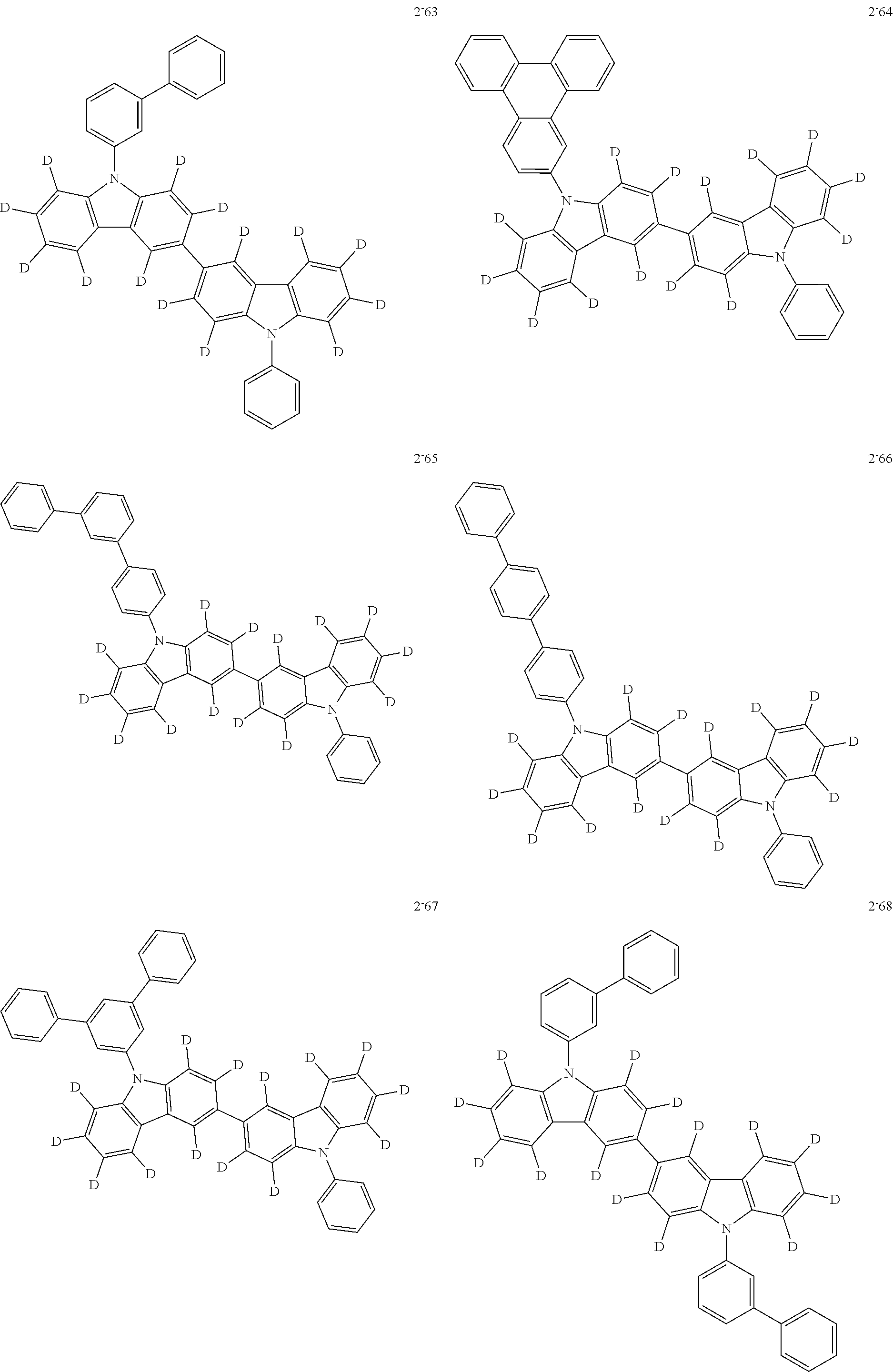

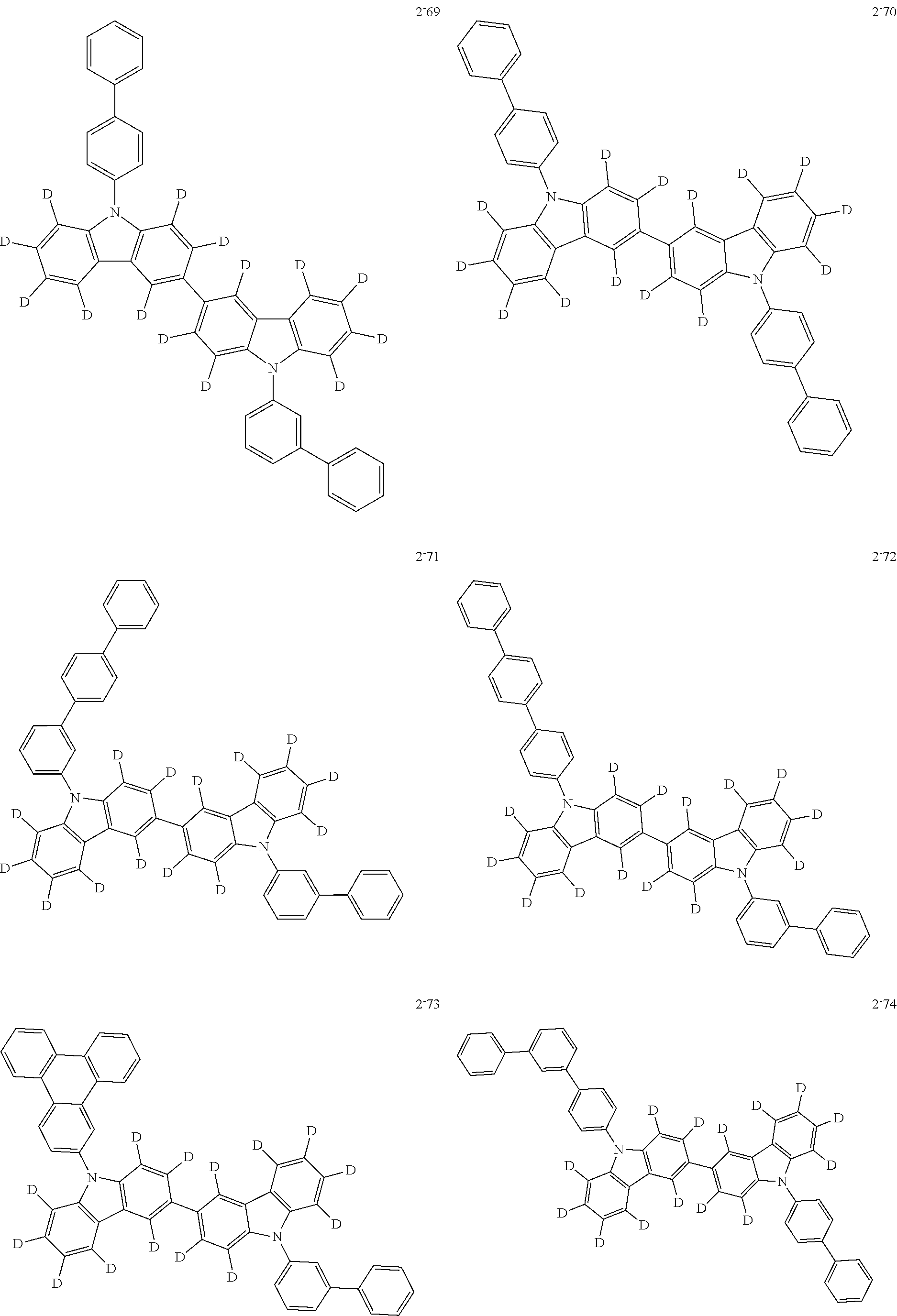

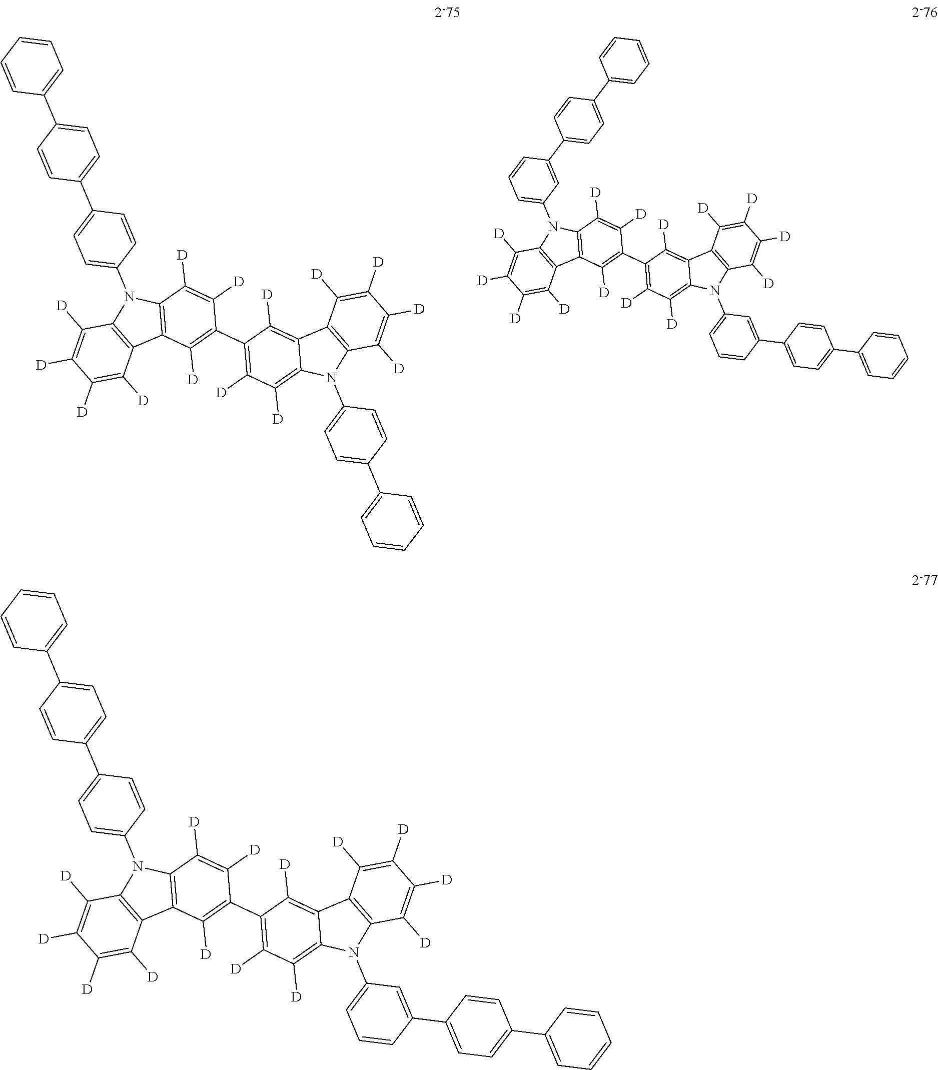

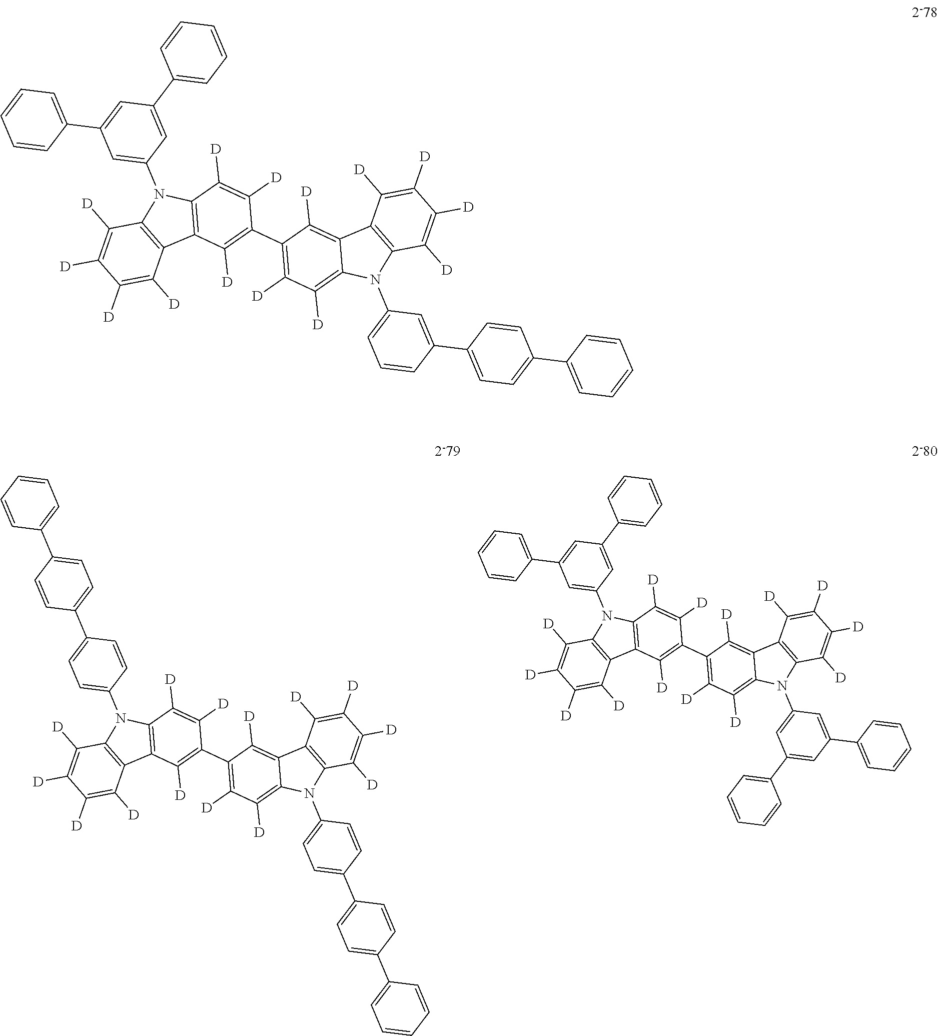

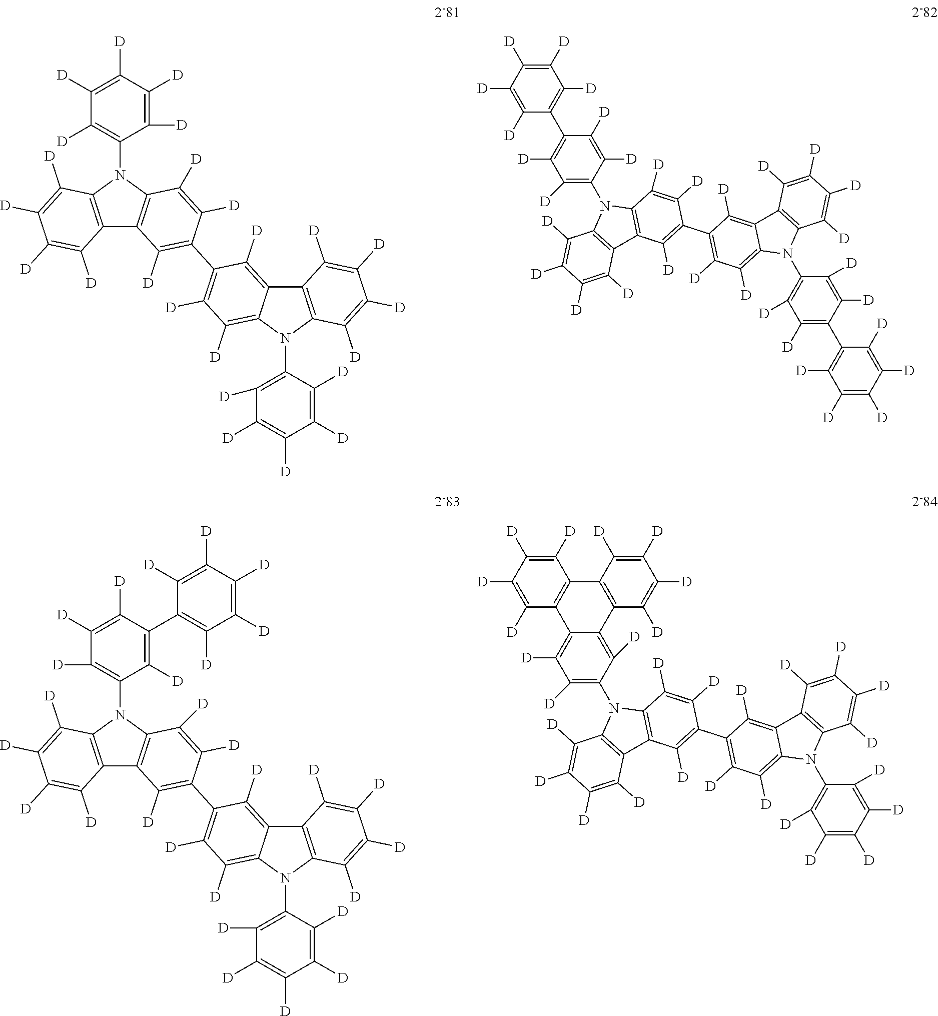

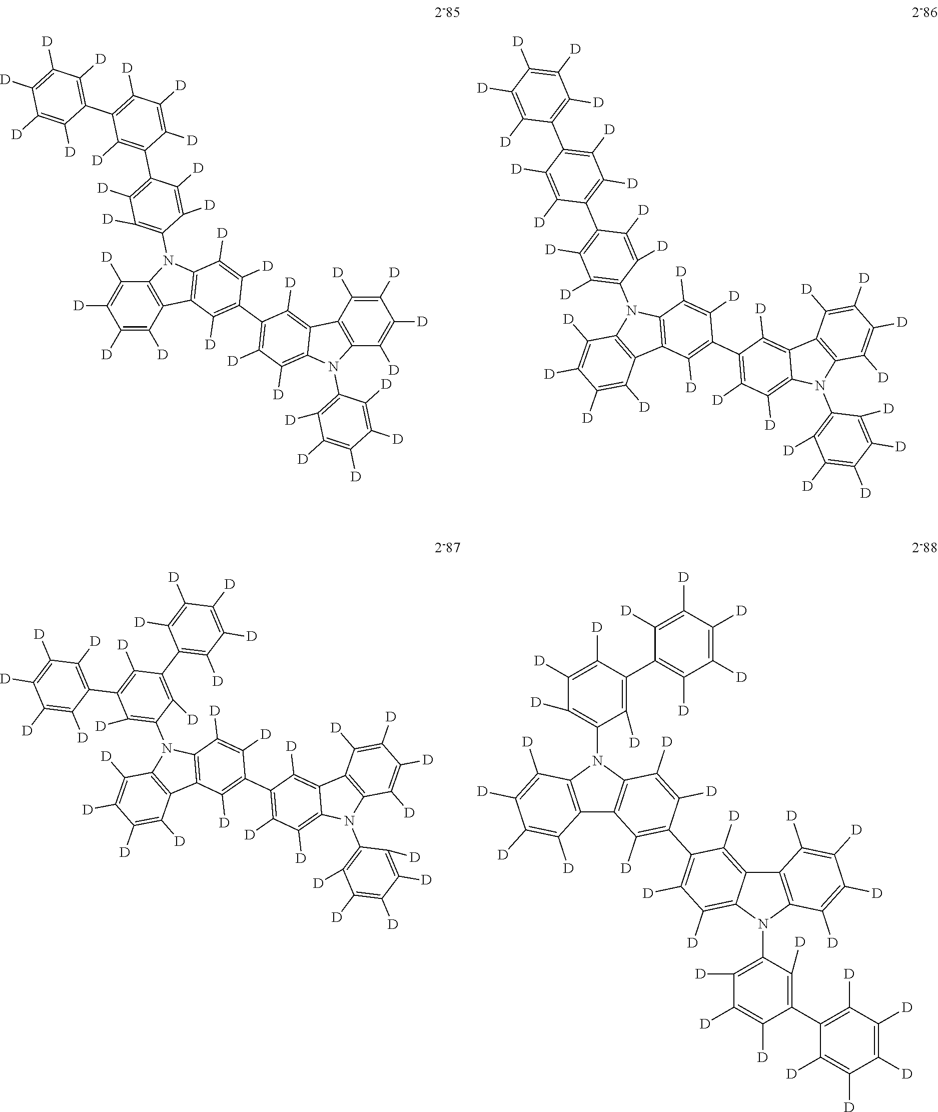

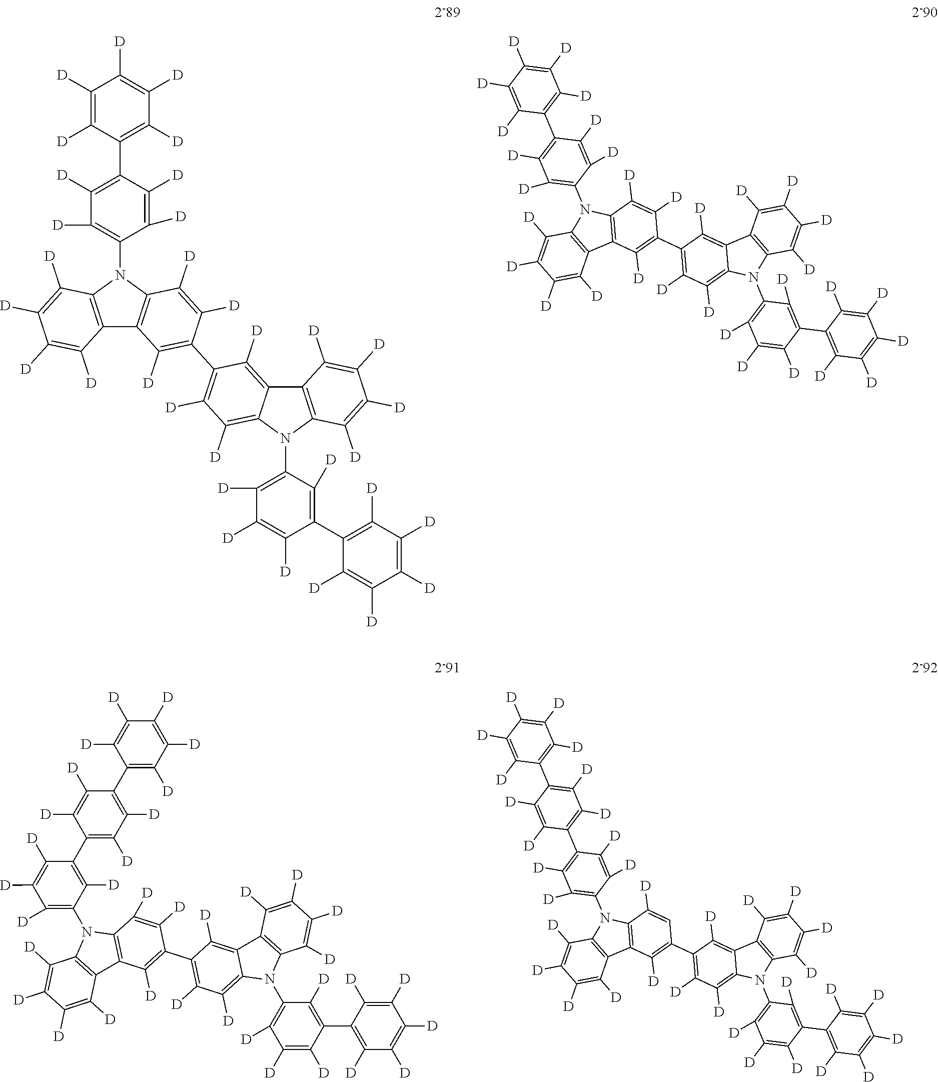

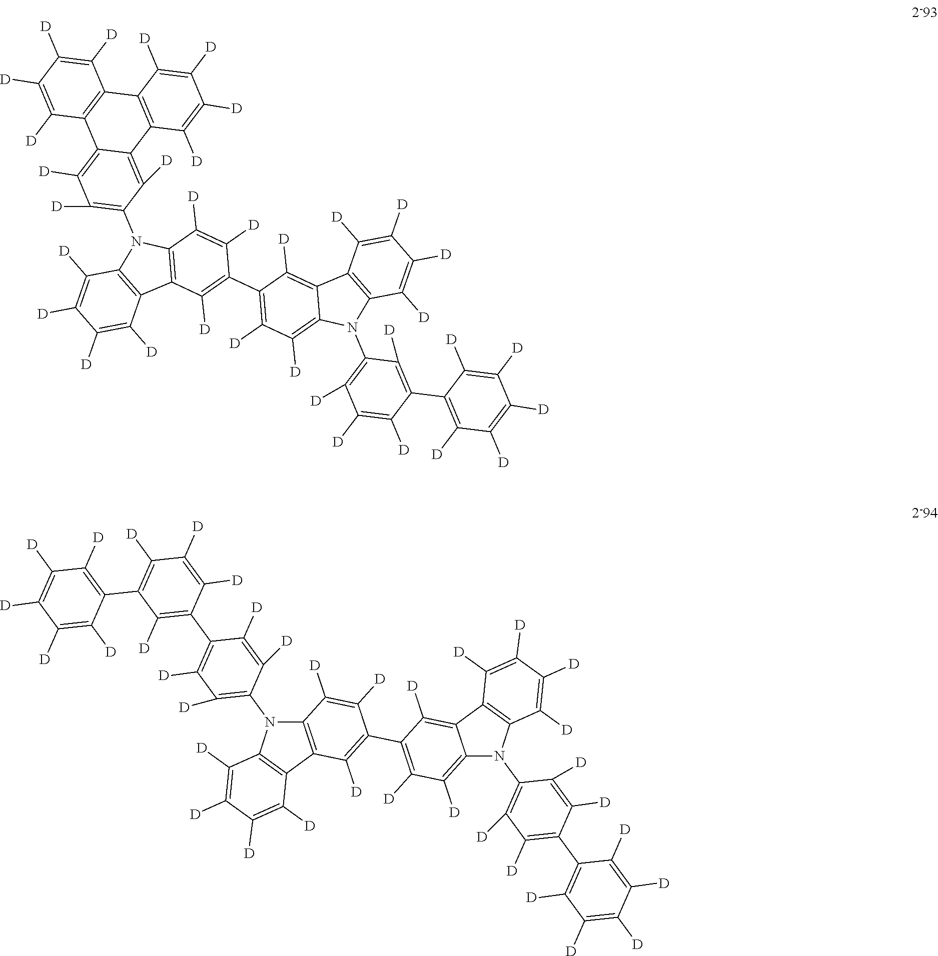

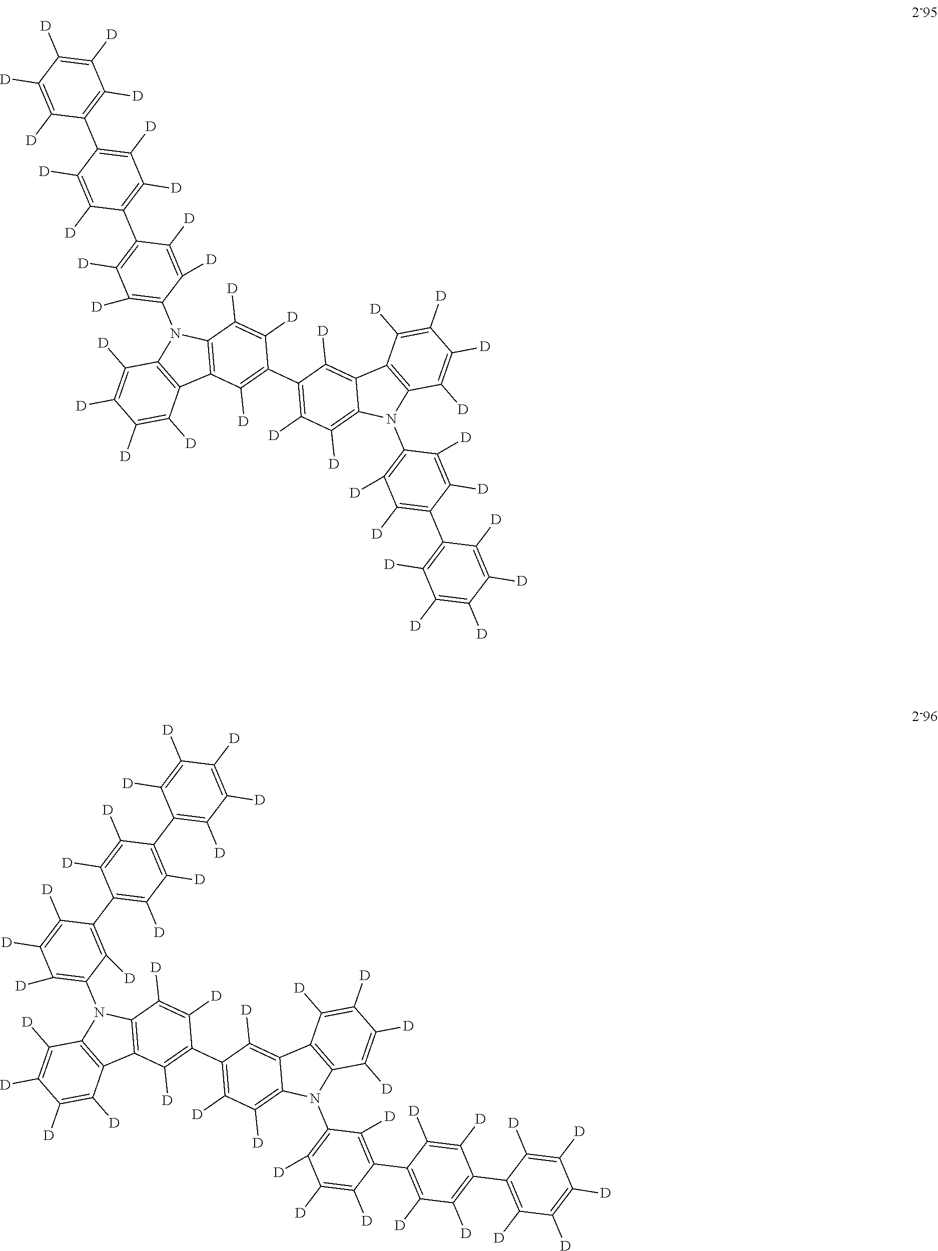

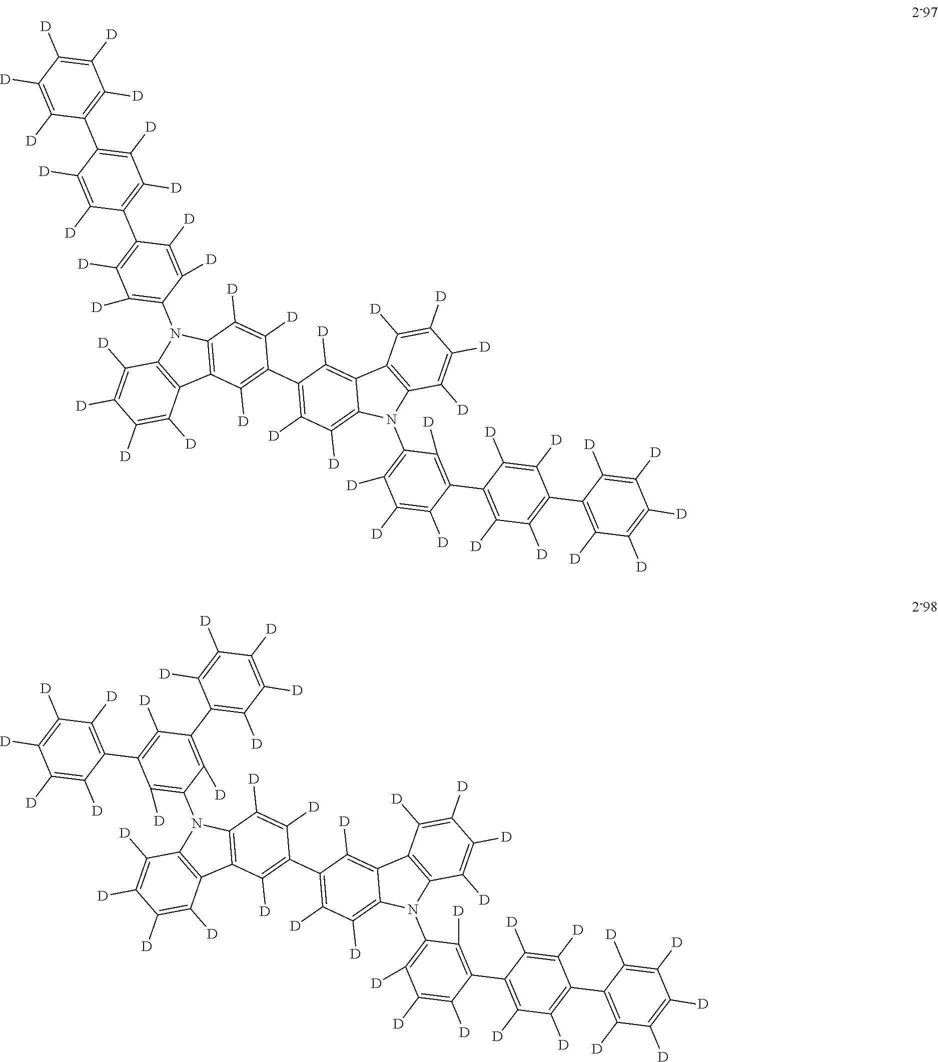

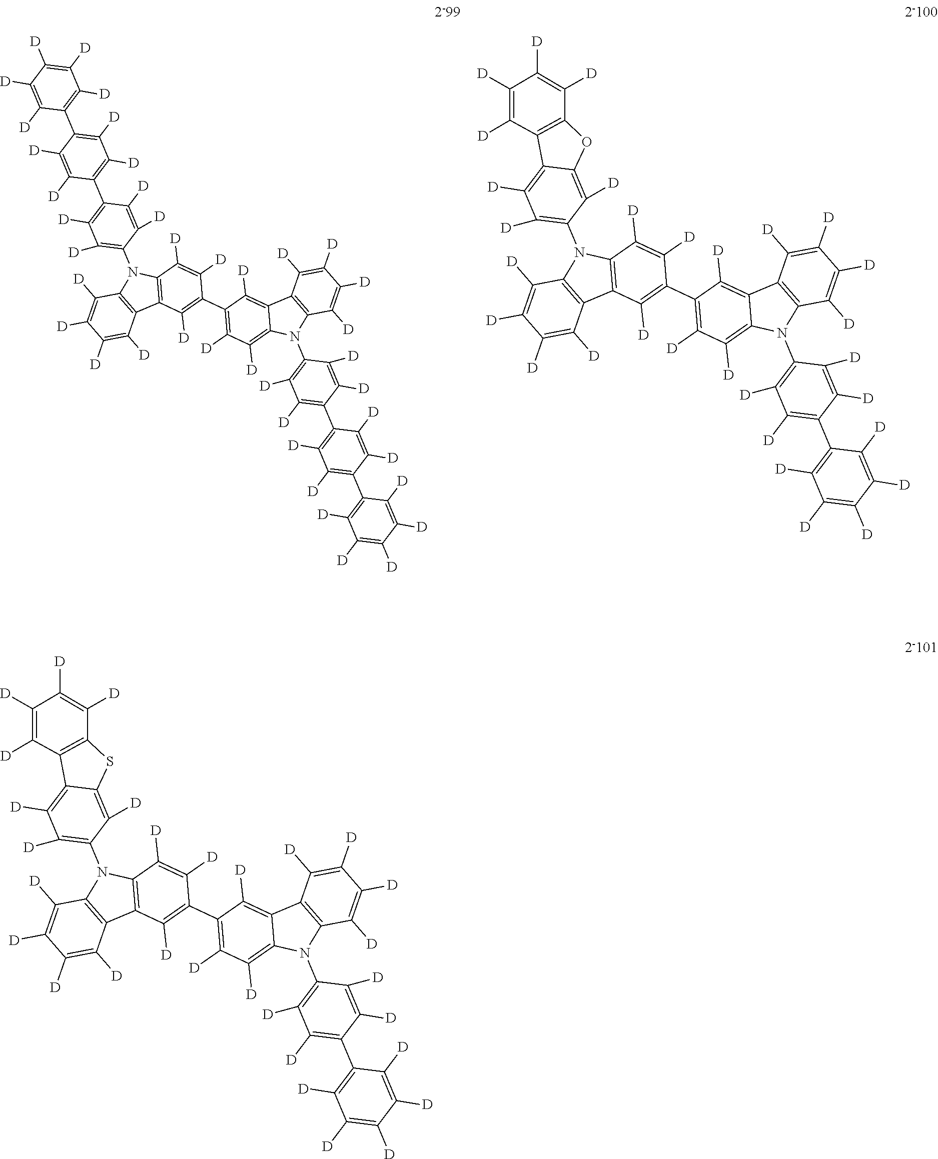

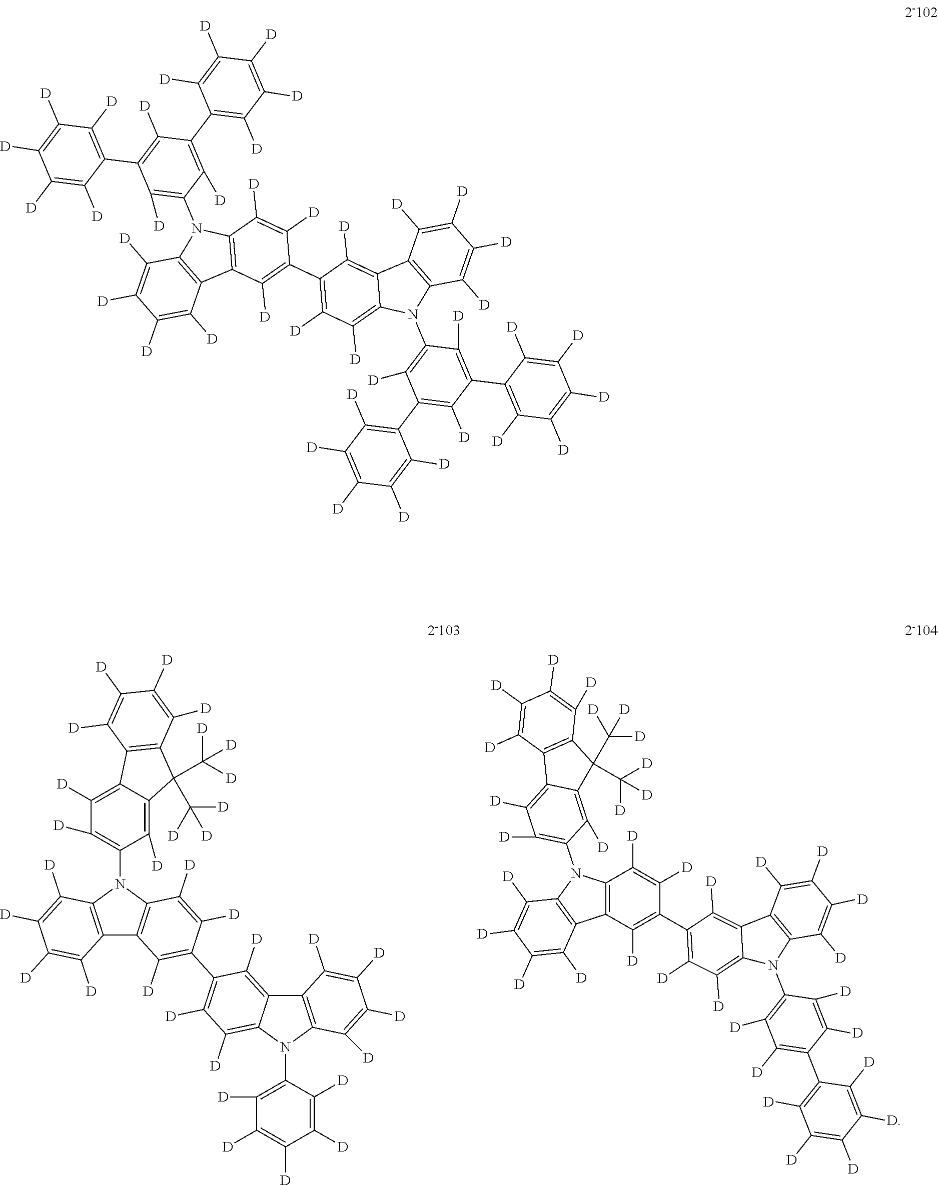

13. The organic light emitting device of claim 9, wherein the heterocyclic compound represented by Chemical Formula 3 is represented by any one of the following compounds:

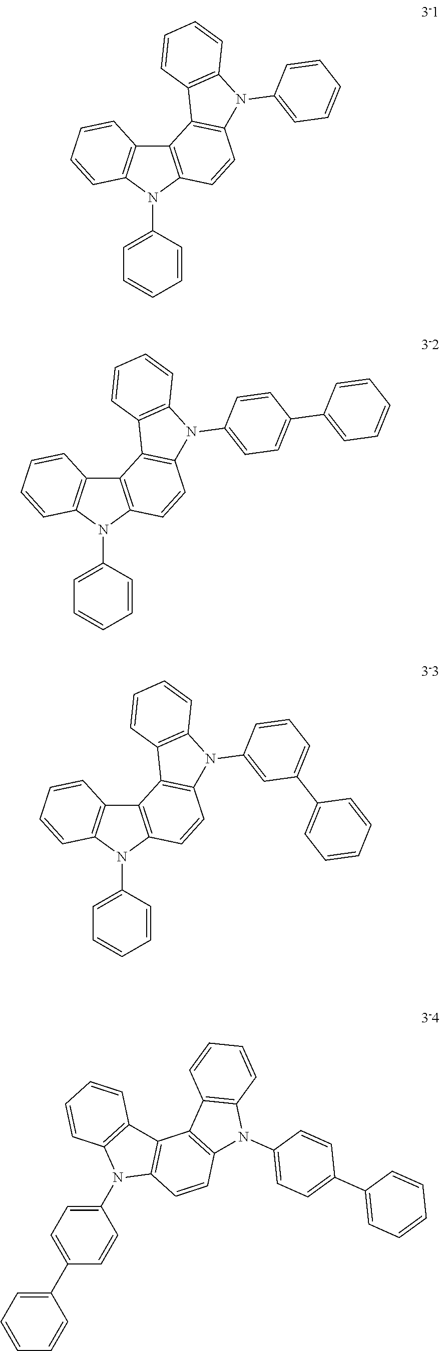 [SMILES: c1ccc(-n2c3ccccc3c3c4c5ccccc5n(-c5ccccc5)c4ccc32)cc1 ; c1ccc(-c2ccc(-n3c4ccccc4c4c5c6ccccc6n(-c6ccccc6)c5ccc43)cc2)cc1 ; c1ccc(-c2cccc(-n3c4ccccc4c4c5c6ccccc6n(-c6ccccc6)c5ccc43)c2)cc1 ; c1ccc(-c2ccc(-n3c4ccccc4c4c5c6ccccc6n(-c6ccc(-c7ccccc7)cc6)c5ccc43)cc2)cc1]

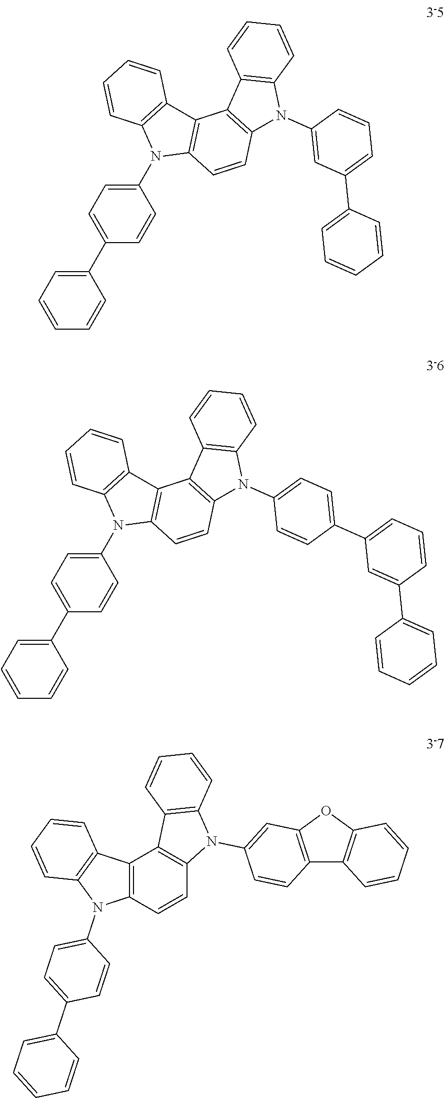 [SMILES: c1ccc(-c2ccc(-n3c4ccccc4c4c5c6ccccc6n(-c6cccc(-c7ccccc7)c6)c5ccc43)cc2)cc1 ; c1ccc(-c2ccc(-n3c4ccccc4c4c5c6ccccc6n(-c6ccc(-c7cccc(-c8ccccc8)c7)cc6)c5ccc43)cc2)cc1 ; c1ccc(-c2ccc(-n3c4ccccc4c4c5c6ccccc6n(-c6ccc7c(c6)oc6ccccc67)c5ccc43)cc2)cc1]

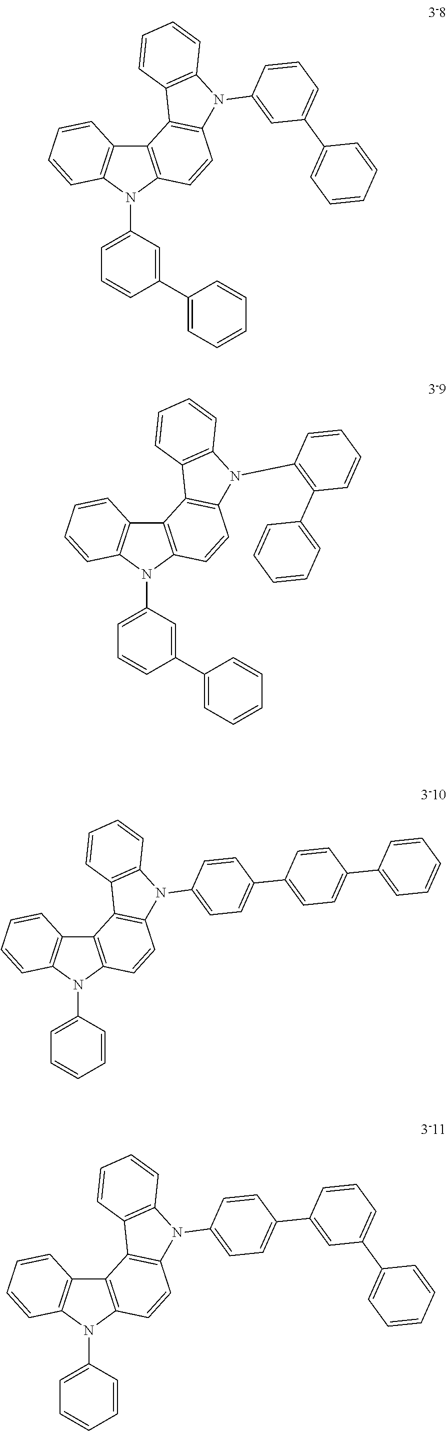 [SMILES: c1ccc(-c2cccc(-n3c4ccccc4c4c5c6ccccc6n(-c6cccc(-c7ccccc7)c6)c5ccc43)c2)cc1 ; c1ccc(-c2cccc(-n3c4ccccc4c4c5c6ccccc6n(-c6ccccc6-c6ccccc6)c5ccc43)c2)cc1 ; c1ccc(-c2ccc(-c3ccc(-n4c5ccccc5c5c6c7ccccc7n(-c7ccccc7)c6ccc54)cc3)cc2)cc1 ; c1ccc(-c2cccc(-c3ccc(-n4c5ccccc5c5c6c7ccccc7n(-c7ccccc7)c6ccc54)cc3)c2)cc1]

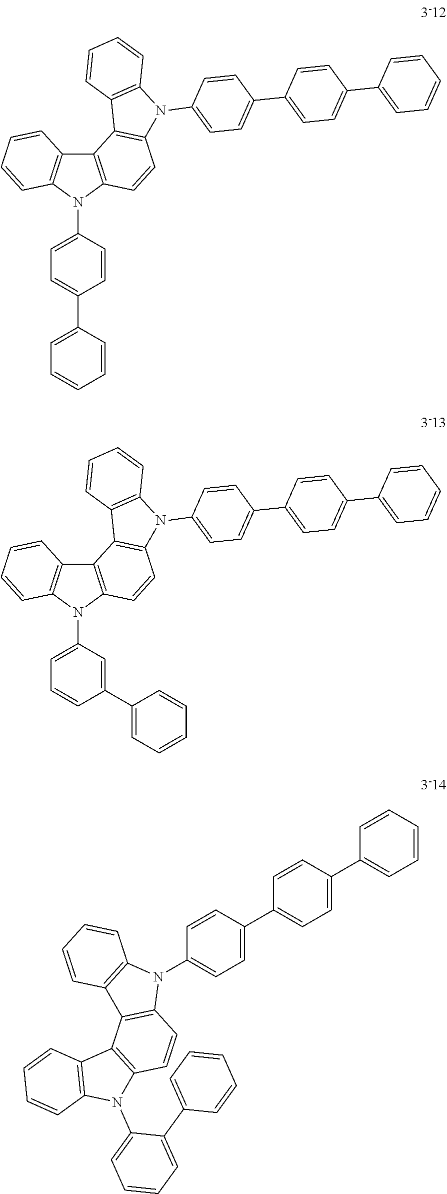 [SMILES: c1ccc(-c2ccc(-c3ccc(-n4c5ccccc5c5c6c7ccccc7n(-c7ccc(-c8ccccc8)cc7)c6ccc54)cc3)cc2)cc1 ; c1ccc(-c2ccc(-c3ccc(-n4c5ccccc5c5c6c7ccccc7n(-c7cccc(-c8ccccc8)c7)c6ccc54)cc3)cc2)cc1 ; c1ccc(-c2ccc(-c3ccc(-n4c5ccccc5c5c6c7ccccc7n(-c7ccccc7-c7ccccc7)c6ccc54)cc3)cc2)cc1]

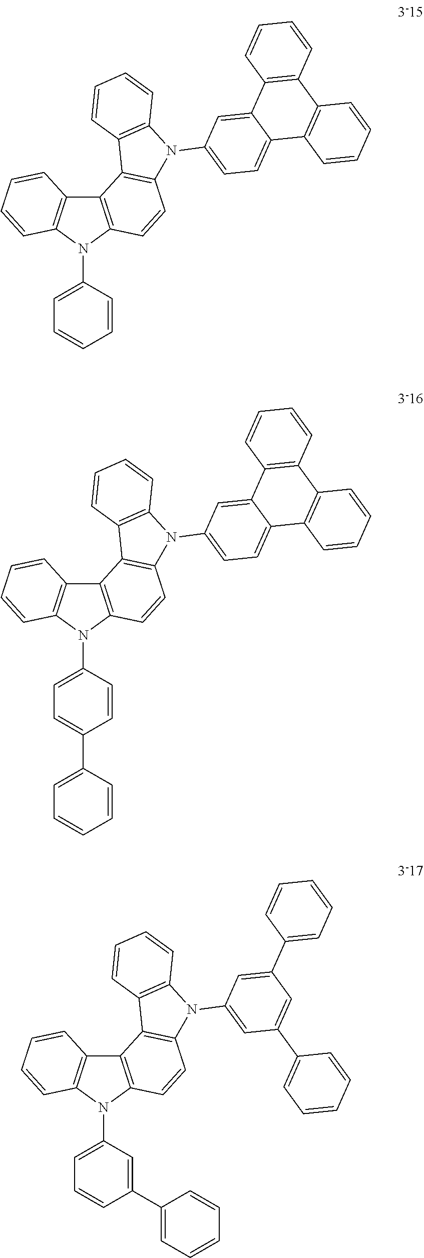 [SMILES: c1ccc(-n2c3ccccc3c3c4c5ccccc5n(-c5ccc6c7ccccc7c7ccccc7c6c5)c4ccc32)cc1 ; c1ccc(-c2ccc(-n3c4ccccc4c4c5c6ccccc6n(-c6ccc7c8ccccc8c8ccccc8c7c6)c5ccc43)cc2)cc1 ; c1ccc(-c2cccc(-n3c4ccccc4c4c5c6ccccc6n(-c6cc(-c7ccccc7)cc(-c7ccccc7)c6)c5ccc43)c2)cc1]

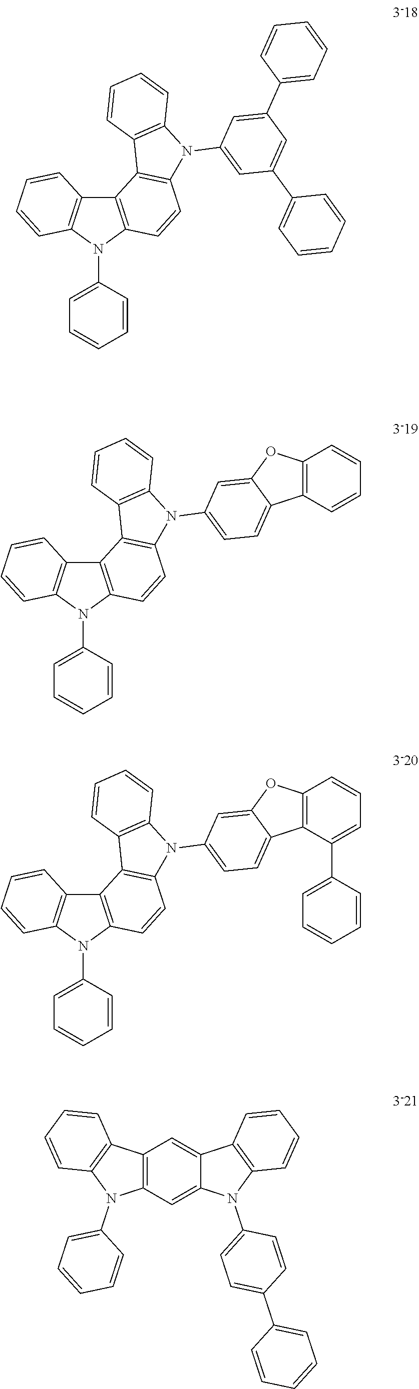 [SMILES: c1ccc(-c2cc(-c3ccccc3)cc(-n3c4ccccc4c4c5c6ccccc6n(-c6ccccc6)c5ccc43)c2)cc1 ; c1ccc(-n2c3ccccc3c3c4c5ccccc5n(-c5ccc6c(c5)oc5ccccc56)c4ccc32)cc1 ; c1ccc(-c2cccc3oc4cc(-n5c6ccccc6c6c7c8ccccc8n(-c8ccccc8)c7ccc65)ccc4c23)cc1 ; c1ccc(-c2ccc(-n3c4ccccc4c4cc5c6ccccc6n(-c6ccccc6)c5cc43)cc2)cc1]

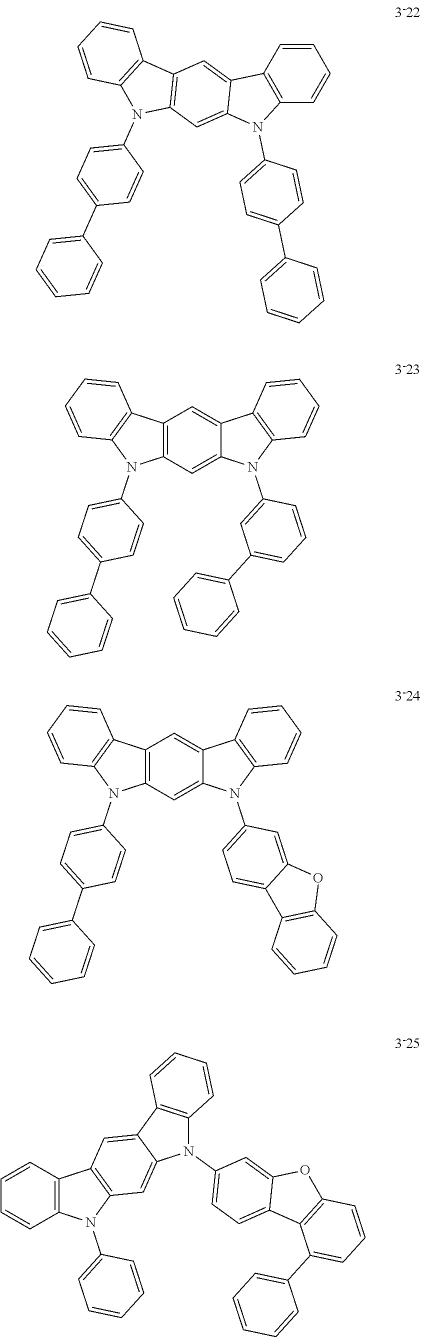 [SMILES: c1ccc(-c2ccc(-n3c4ccccc4c4cc5c6ccccc6n(-c6ccc(-c7ccccc7)cc6)c5cc43)cc2)cc1 ; c1ccc(-c2ccc(-n3c4ccccc4c4cc5c6ccccc6n(-c6cccc(-c7ccccc7)c6)c5cc43)cc2)cc1 ; c1ccc(-c2ccc(-n3c4ccccc4c4cc5c6ccccc6n(-c6ccc7c(c6)oc6ccccc67)c5cc43)cc2)cc1 ; c1ccc(-c2cccc3oc4cc(-n5c6ccccc6c6cc7c8ccccc8n(-c8ccccc8)c7cc65)ccc4c23)cc1]

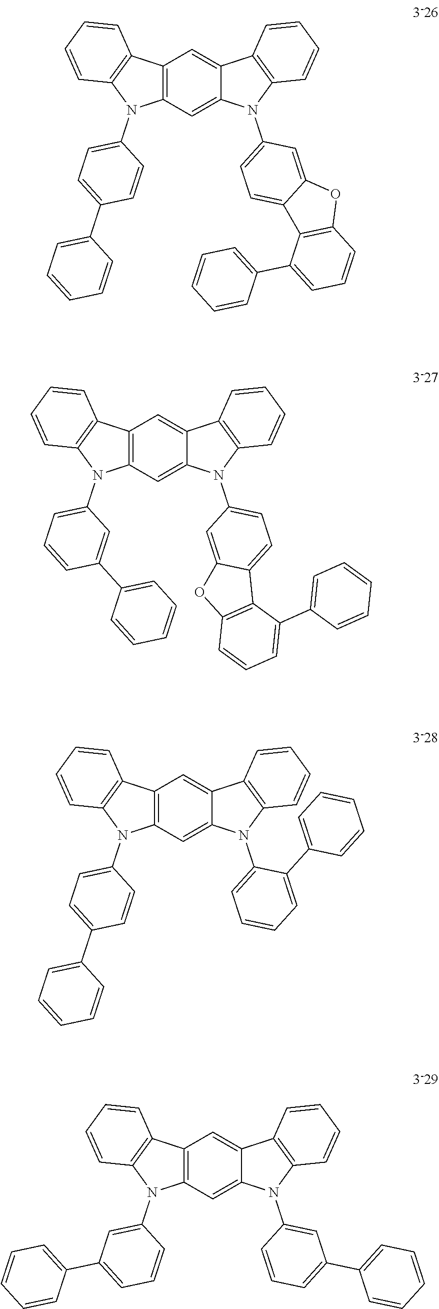 [SMILES: c1ccc(-c2ccc(-n3c4ccccc4c4cc5c6ccccc6n(-c6ccc7c(c6)oc6cccc(-c8ccccc8)c67)c5cc43)cc2)cc1 ; c1ccc(-c2cccc(-n3c4ccccc4c4cc5c6ccccc6n(-c6ccc7c(c6)oc6cccc(-c8ccccc8)c67)c5cc43)c2)cc1 ; c1ccc(-c2ccc(-n3c4ccccc4c4cc5c6ccccc6n(-c6ccccc6-c6ccccc6)c5cc43)cc2)cc1 ; c1ccc(-c2cccc(-n3c4ccccc4c4cc5c6ccccc6n(-c6cccc(-c7ccccc7)c6)c5cc43)c2)cc1]

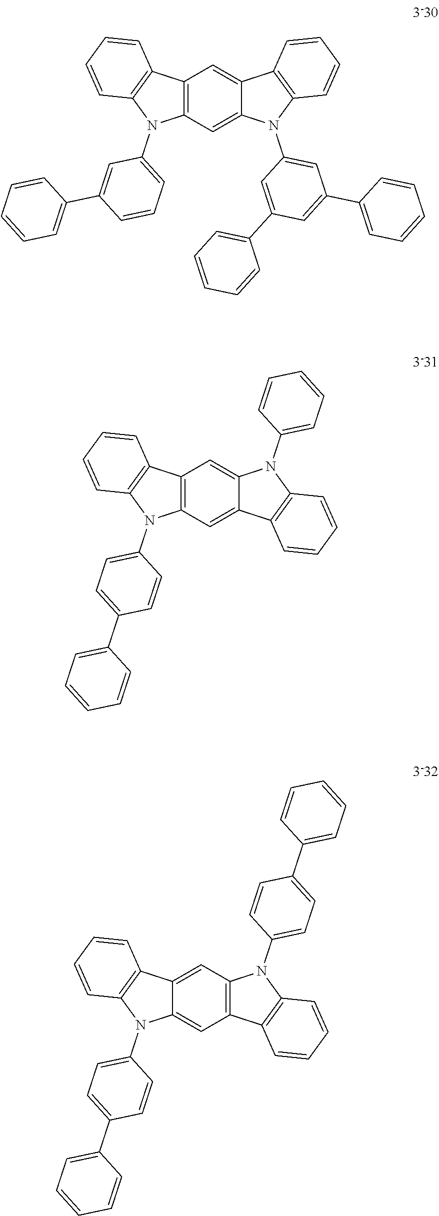 [SMILES: c1ccc(-c2cccc(-n3c4ccccc4c4cc5c6ccccc6n(-c6cc(-c7ccccc7)cc(-c7ccccc7)c6)c5cc43)c2)cc1 ; c1ccc(-c2ccc(-n3c4ccccc4c4cc5c(cc43)c3ccccc3n5-c3ccccc3)cc2)cc1 ; c1ccc(-c2ccc(-n3c4ccccc4c4cc5c(cc43)c3ccccc3n5-c3ccc(-c4ccccc4)cc3)cc2)cc1]

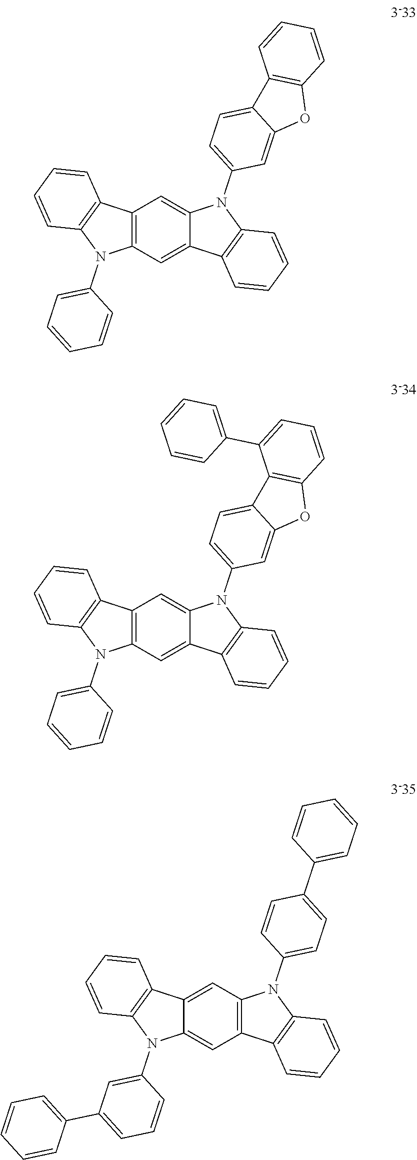 [SMILES: c1ccc(-n2c3ccccc3c3cc4c(cc32)c2ccccc2n4-c2ccc3c(c2)oc2ccccc23)cc1 ; c1ccc(-c2cccc3oc4cc(-n5c6ccccc6c6cc7c(cc65)c5ccccc5n7-c5ccccc5)ccc4c23)cc1 ; c1ccc(-c2ccc(-n3c4ccccc4c4cc5c(cc43)c3ccccc3n5-c3cccc(-c4ccccc4)c3)cc2)cc1]

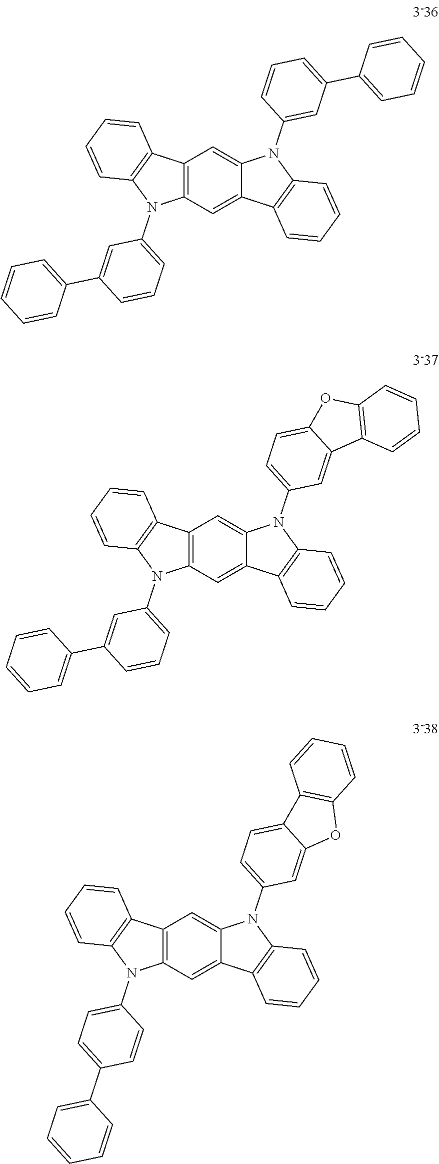 [SMILES: c1ccc(-c2cccc(-n3c4ccccc4c4cc5c(cc43)c3ccccc3n5-c3cccc(-c4ccccc4)c3)c2)cc1 ; c1ccc(-c2cccc(-n3c4ccccc4c4cc5c(cc43)c3ccccc3n5-c3ccc4oc5ccccc5c4c3)c2)cc1 ; c1ccc(-c2ccc(-n3c4ccccc4c4cc5c(cc43)c3ccccc3n5-c3ccc4c(c3)oc3ccccc34)cc2)cc1]

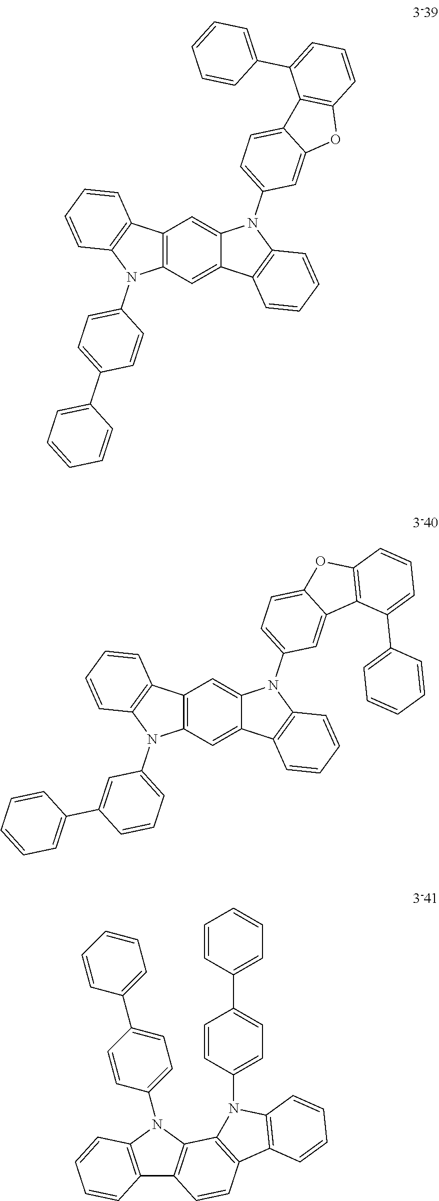

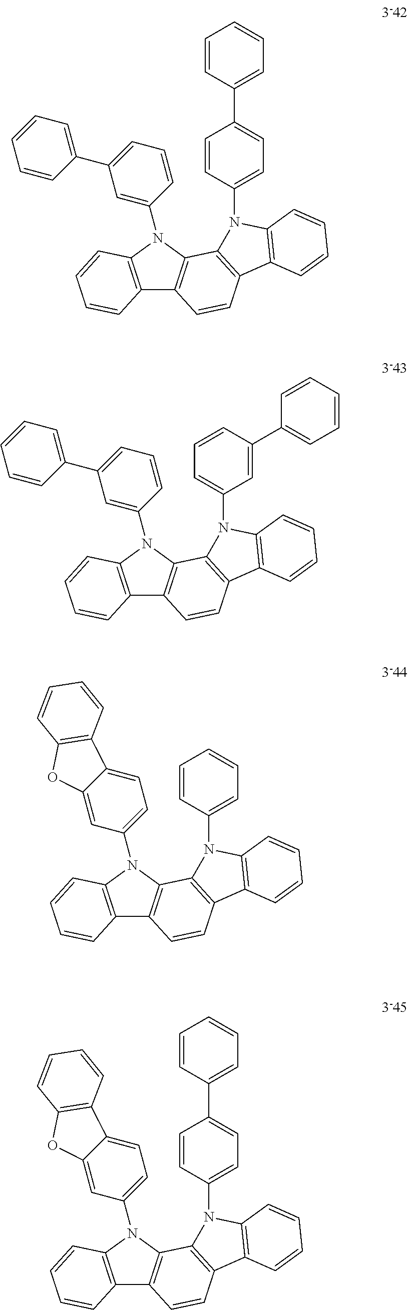 [SMILES: c1ccc(-c2ccc(-n3c4ccccc4c4ccc5c6ccccc6n(-c6cccc(-c7ccccc7)c6)c5c43)cc2)cc1 ; c1ccc(-c2cccc(-n3c4ccccc4c4ccc5c6ccccc6n(-c6cccc(-c7ccccc7)c6)c5c43)c2)cc1 ; c1ccc(-n2c3ccccc3c3ccc4c5ccccc5n(-c5ccc6c(c5)oc5ccccc56)c4c32)cc1 ; c1ccc(-c2ccc(-n3c4ccccc4c4ccc5c6ccccc6n(-c6ccc7c(c6)oc6ccccc67)c5c43)cc2)cc1]

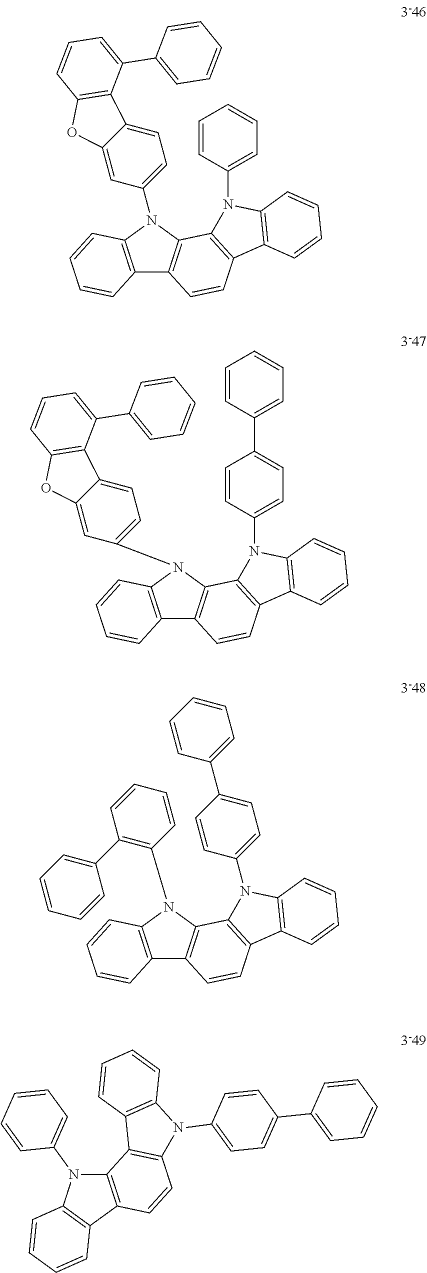 [SMILES: c1ccc(-c2cccc3oc4cc(-n5c6ccccc6c6ccc7c8ccccc8n(-c8ccccc8)c7c65)ccc4c23)cc1 ; c1ccc(-c2ccc(-n3c4ccccc4c4ccc5c6ccccc6n(-c6ccc7c(c6)oc6cccc(-c8ccccc8)c67)c5c43)cc2)cc1 ; c1ccc(-c2ccc(-n3c4ccccc4c4ccc5c6ccccc6n(-c6ccccc6-c6ccccc6)c5c43)cc2)cc1 ; c1ccc(-c2ccc(-n3c4ccccc4c4c3ccc3c5ccccc5n(-c5ccccc5)c34)cc2)cc1]

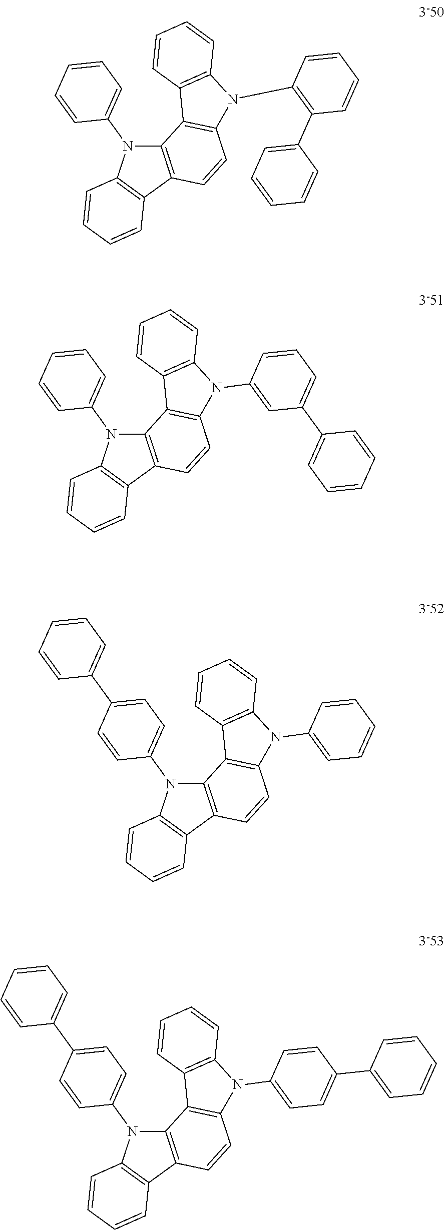 [SMILES: c1ccc(-c2ccccc2-n2c3ccccc3c3c2ccc2c4ccccc4n(-c4ccccc4)c23)cc1 ; c1ccc(-c2cccc(-n3c4ccccc4c4c3ccc3c5ccccc5n(-c5ccccc5)c34)c2)cc1 ; c1ccc(-c2ccc(-n3c4ccccc4c4ccc5c(c6ccccc6n5-c5ccccc5)c43)cc2)cc1 ; c1ccc(-c2ccc(-n3c4ccccc4c4c3ccc3c5ccccc5n(-c5ccc(-c6ccccc6)cc5)c34)cc2)cc1]

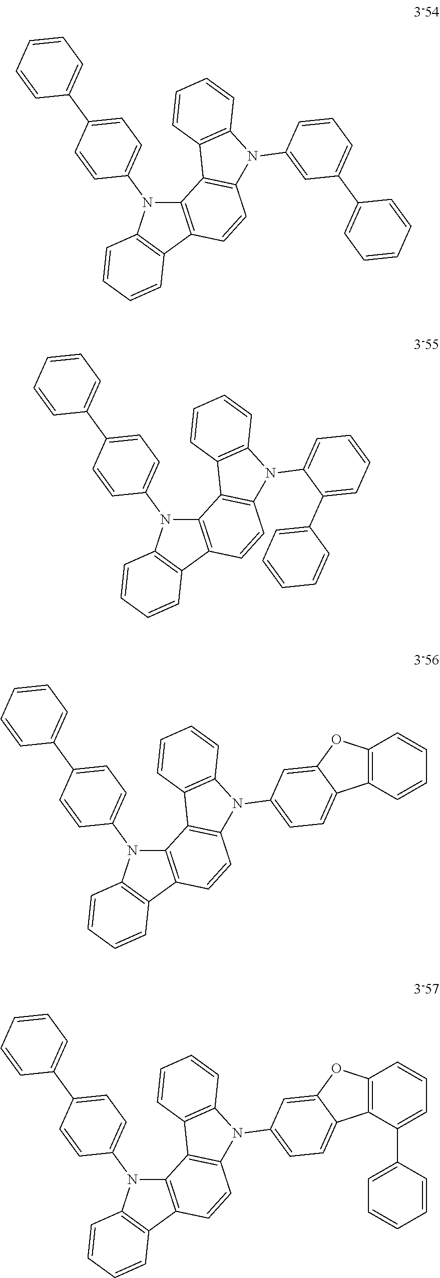 [SMILES: c1ccc(-c2ccc(-n3c4ccccc4c4ccc5c(c6ccccc6n5-c5cccc(-c6ccccc6)c5)c43)cc2)cc1 ; c1ccc(-c2ccc(-n3c4ccccc4c4ccc5c(c6ccccc6n5-c5ccccc5-c5ccccc5)c43)cc2)cc1 ; c1ccc(-c2ccc(-n3c4ccccc4c4ccc5c(c6ccccc6n5-c5ccc6c(c5)oc5ccccc56)c43)cc2)cc1 ; c1ccc(-c2ccc(-n3c4ccccc4c4ccc5c(c6ccccc6n5-c5ccc6c(c5)oc5cccc(-c7ccccc7)c56)c43)cc2)cc1]

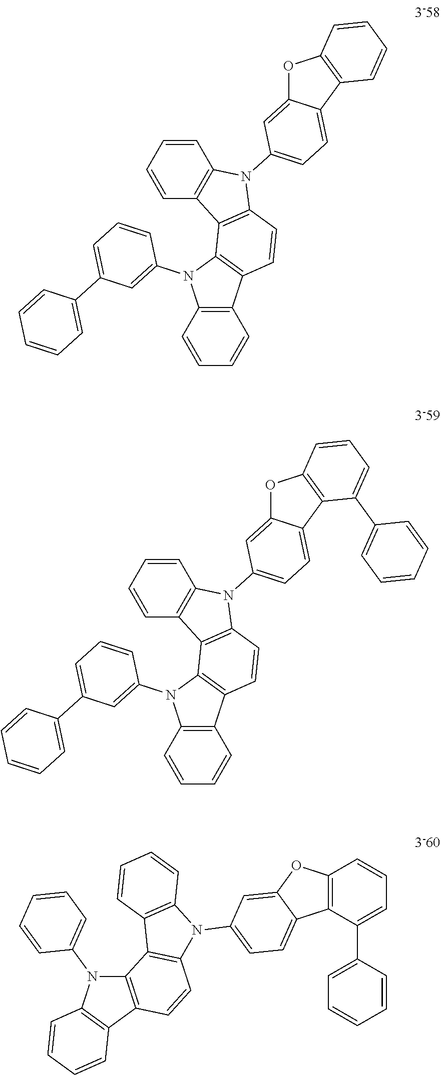 [SMILES: c1ccc(-c2cccc(-n3c4ccccc4c4ccc5c(c6ccccc6n5-c5ccc6c(c5)oc5ccccc56)c43)c2)cc1 ; c1ccc(-c2cccc(-n3c4ccccc4c4ccc5c(c6ccccc6n5-c5ccc6c(c5)oc5cccc(-c7ccccc7)c56)c43)c2)cc1 ; c1ccc(-c2cccc3oc4cc(-n5c6ccccc6c6c5ccc5c7ccccc7n(-c7ccccc7)c56)ccc4c23)cc1]

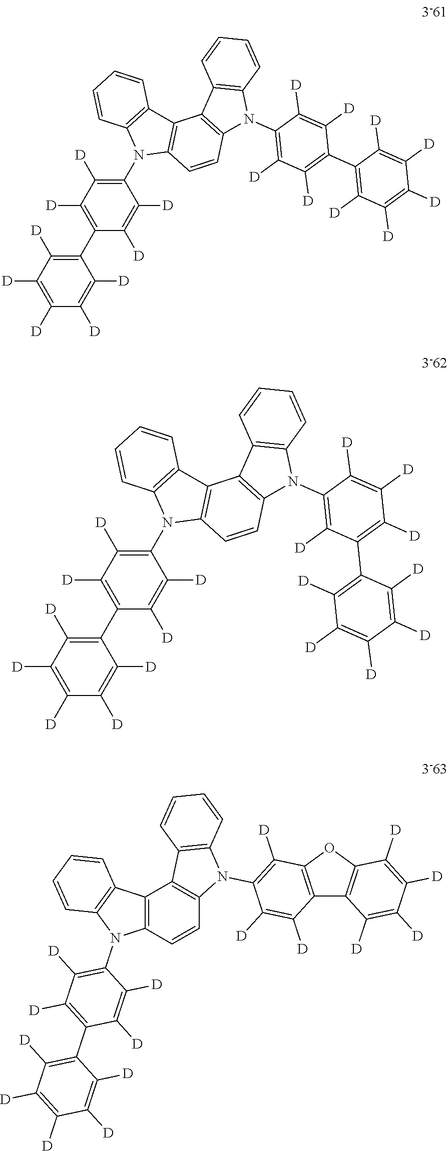

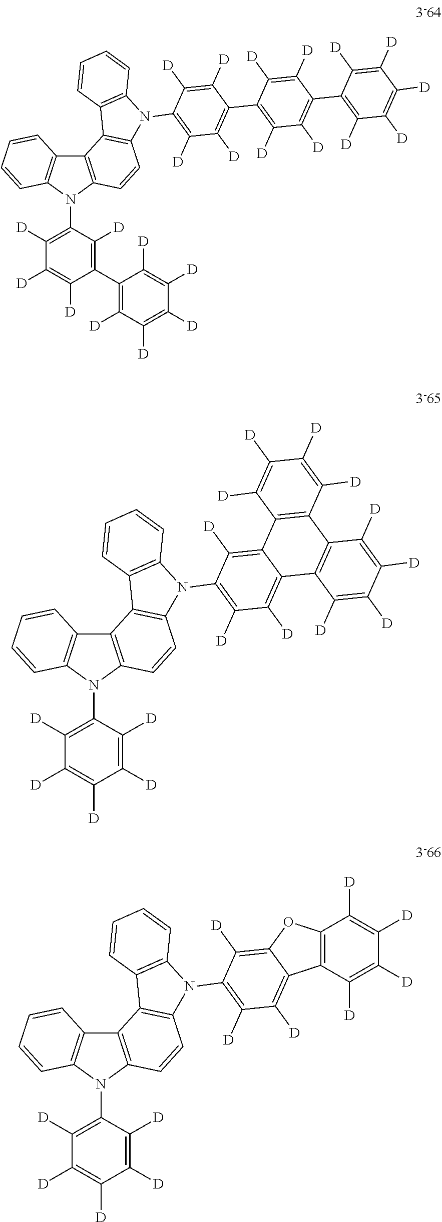

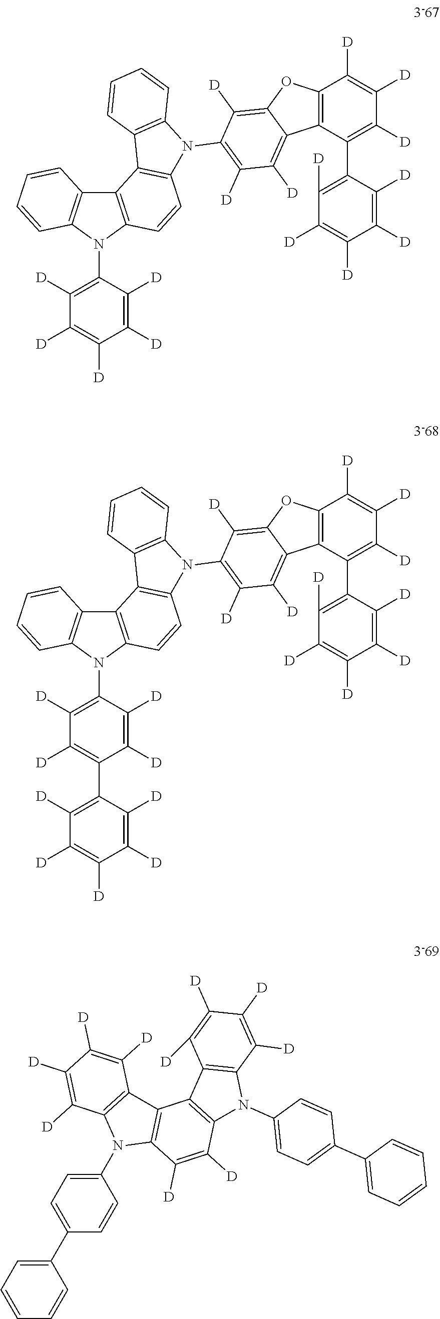 [SMILES: [2H]c1c([2H])c([2H])c(-c2c([2H])c([2H])c([2H])c3oc4c([2H])c(-n5c6ccccc6c6c7c8ccccc8n(-c8c([2H])c([2H])c([2H])c([2H])c8[2H])c7ccc65)c([2H])c([2H])c4c23)c([2H])c1[2H] ; [2H]c1c([2H])c([2H])c(-c2c([2H])c([2H])c(-n3c4ccccc4c4c5c6ccccc6n(-c6c([2H])c([2H])c7c(oc8c([2H])c([2H])c([2H])c(-c9c([2H])c([2H])c([2H])c([2H])c9[2H])c87)c6[2H])c5ccc43)c([2H])c2[2H])c([2H])c1[2H] ; [2H]c1c([2H])c([2H])c2c(c1[2H])c1c3c4c([2H])c([2H])c([2H])c([2H])c4n(-c4ccc(-c5ccccc5)cc4)c3c([2H])c([2H])c1n2-c1ccc(-c2ccccc2)cc1]

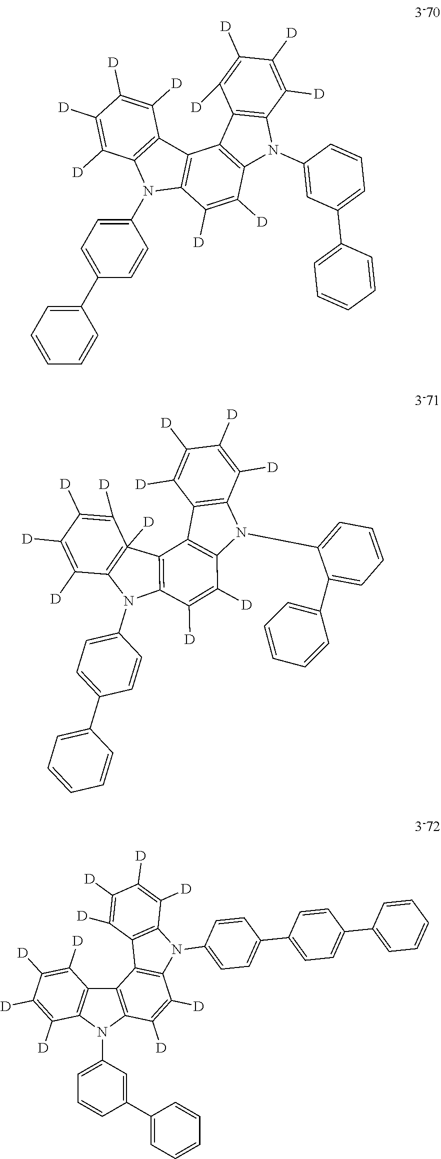

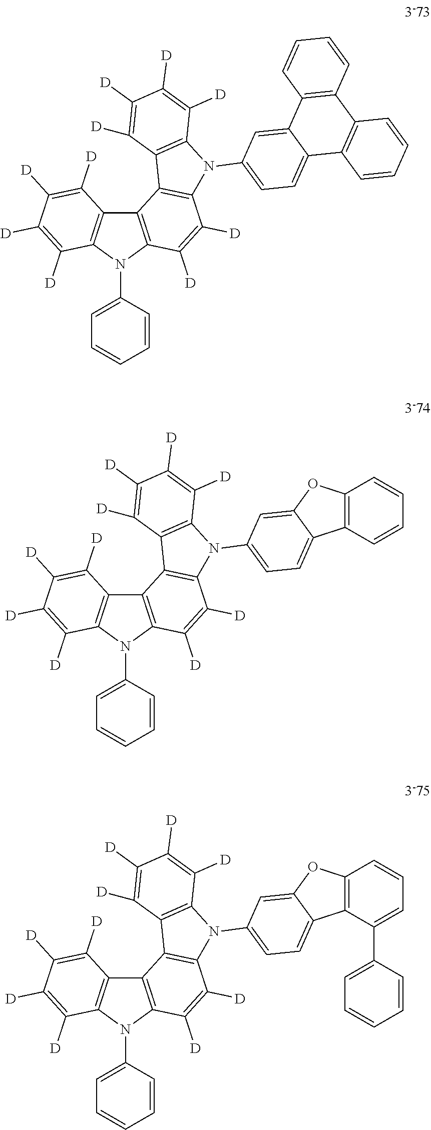 [SMILES: [2H]c1c([2H])c([2H])c2c(c1[2H])c1c3c4c([2H])c([2H])c([2H])c([2H])c4n(-c4ccc5c6ccccc6c6ccccc6c5c4)c3c([2H])c([2H])c1n2-c1ccccc1 ; [2H]c1c([2H])c([2H])c2c(c1[2H])c1c3c4c([2H])c([2H])c([2H])c([2H])c4n(-c4ccc5c(c4)oc4ccccc45)c3c([2H])c([2H])c1n2-c1ccccc1 ; [2H]c1c([2H])c([2H])c2c(c1[2H])c1c3c4c([2H])c([2H])c([2H])c([2H])c4n(-c4ccc5c(c4)oc4cccc(-c6ccccc6)c45)c3c([2H])c([2H])c1n2-c1ccccc1]

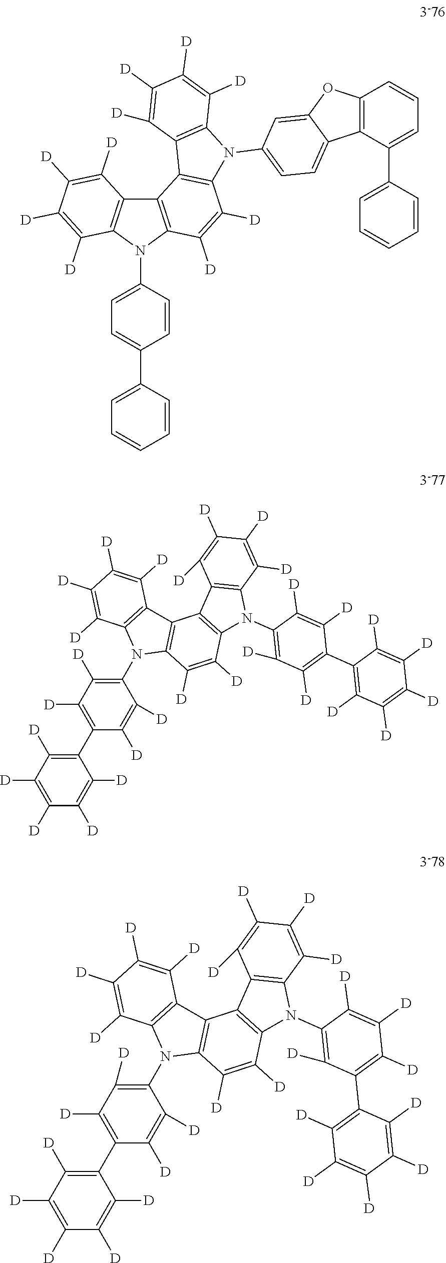

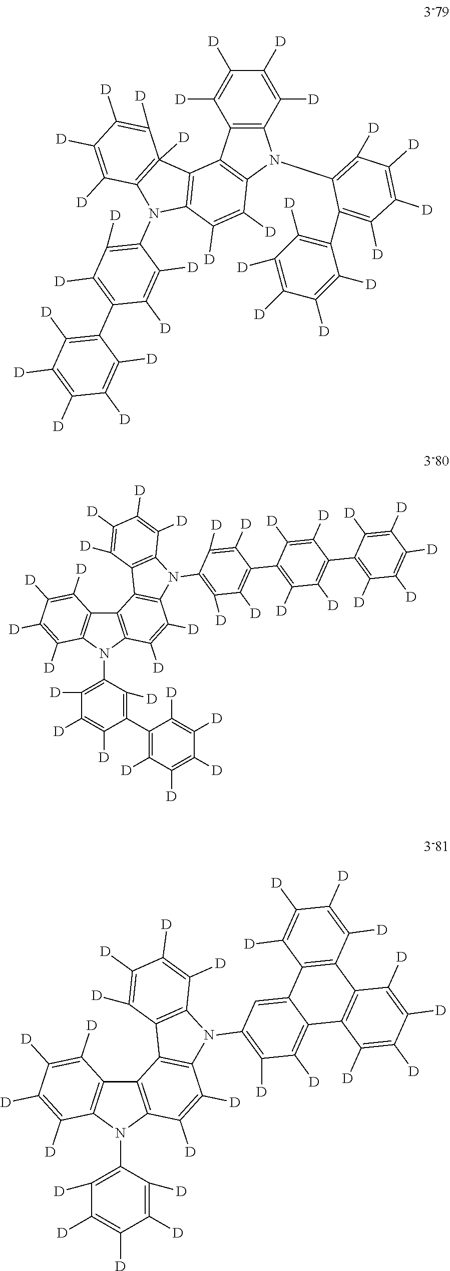

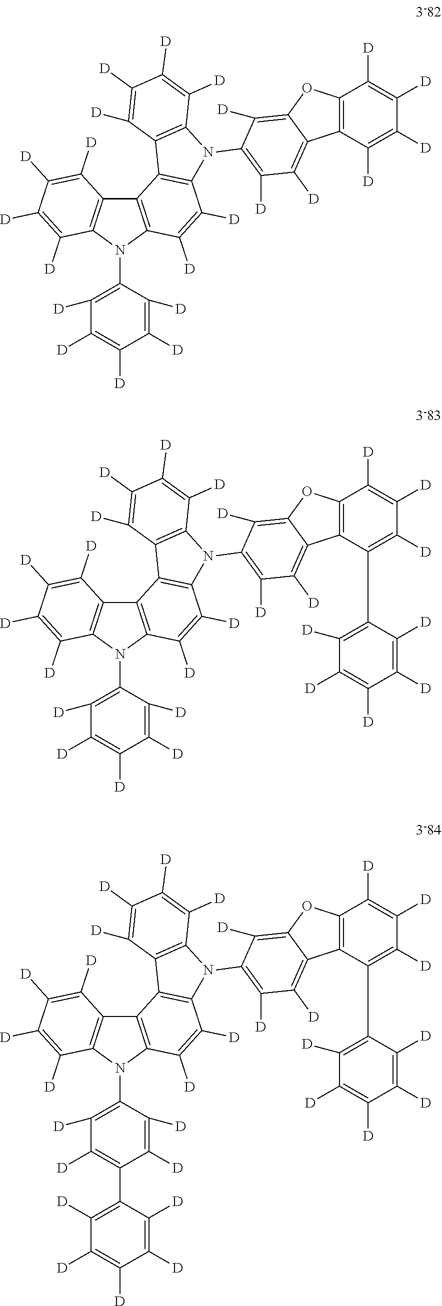

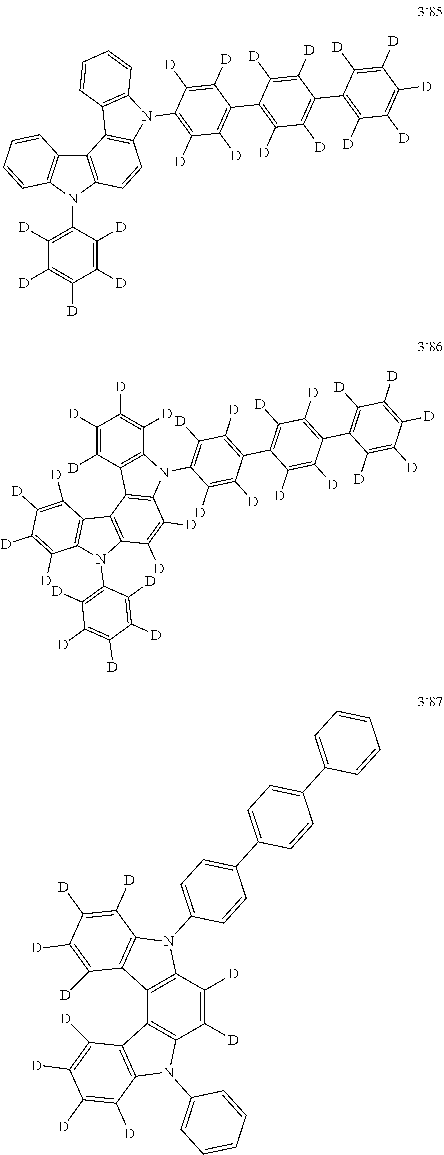

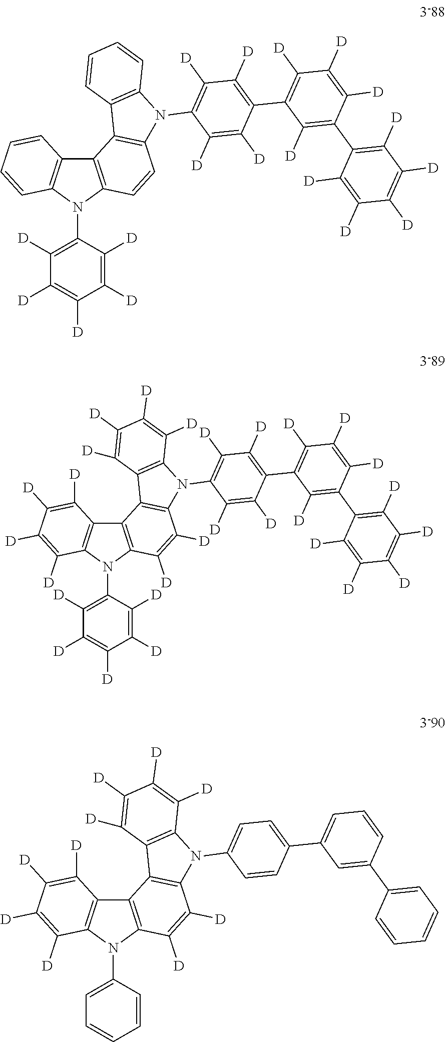

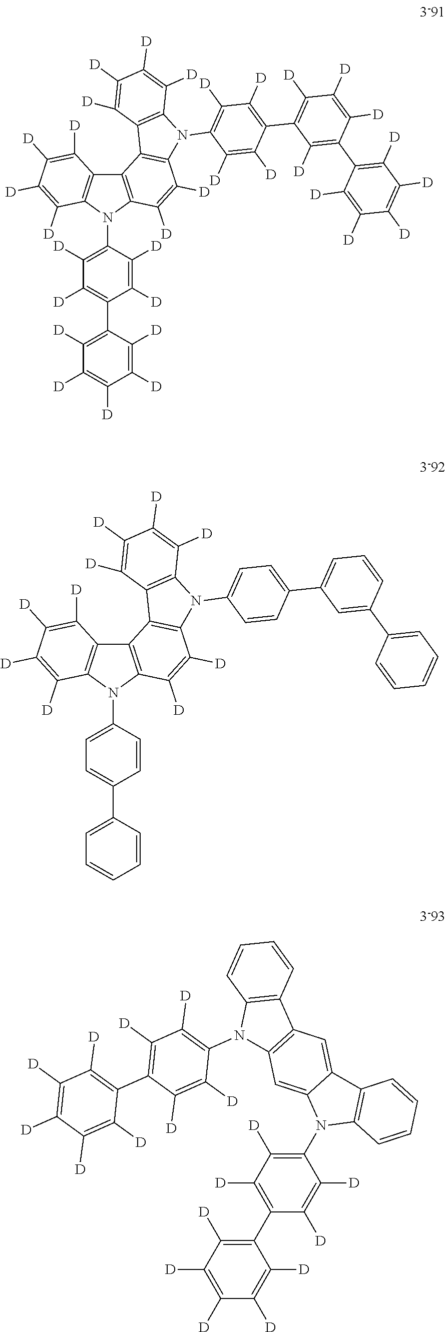

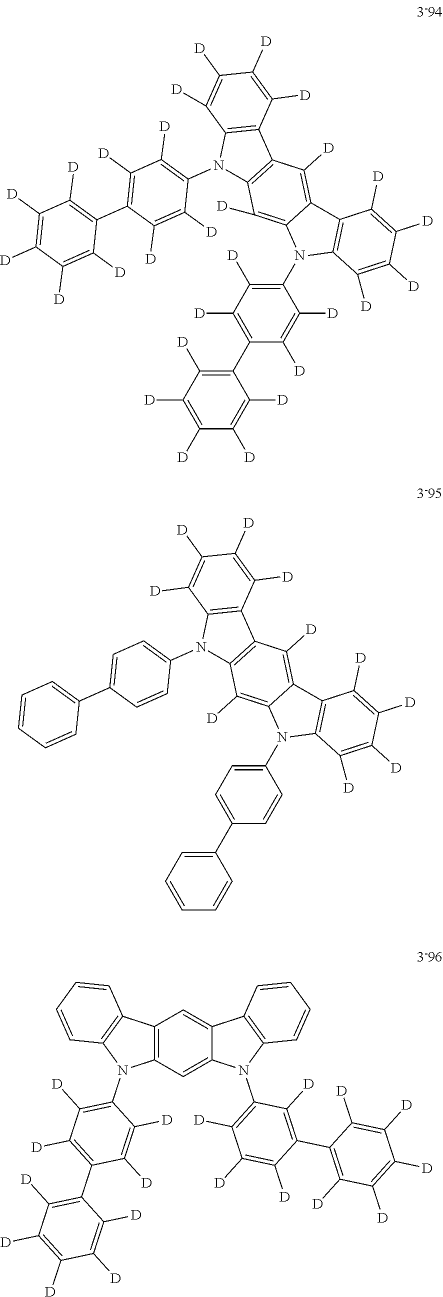

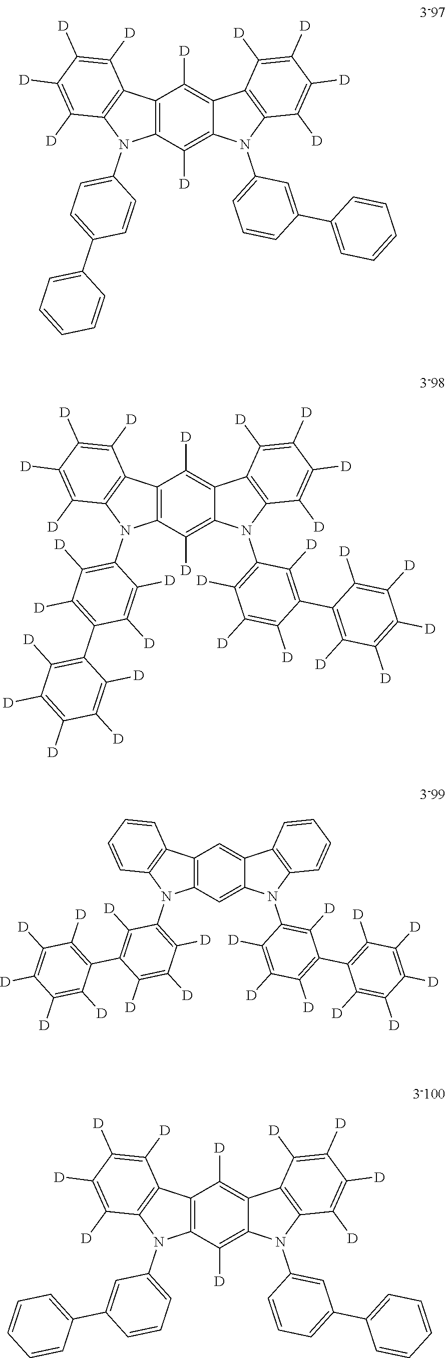

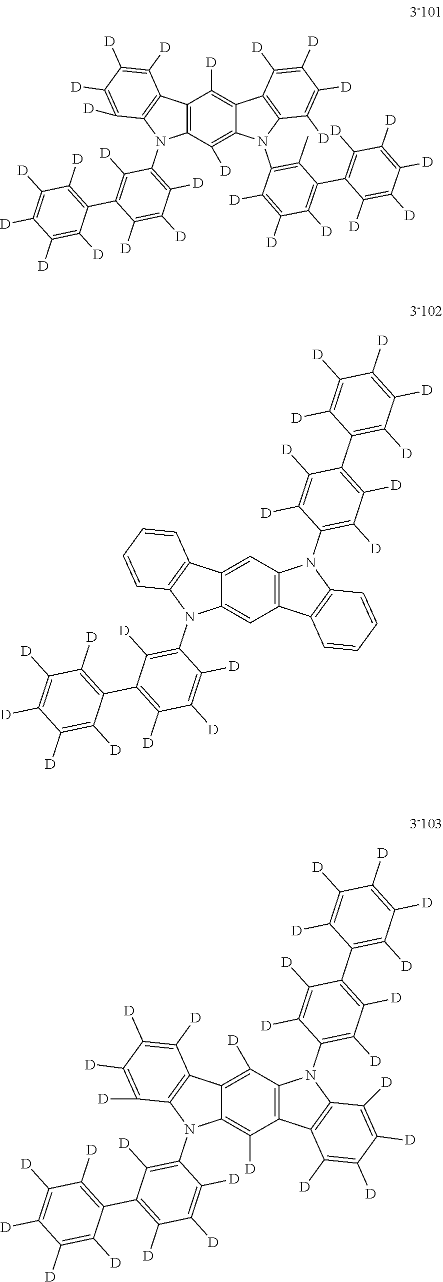

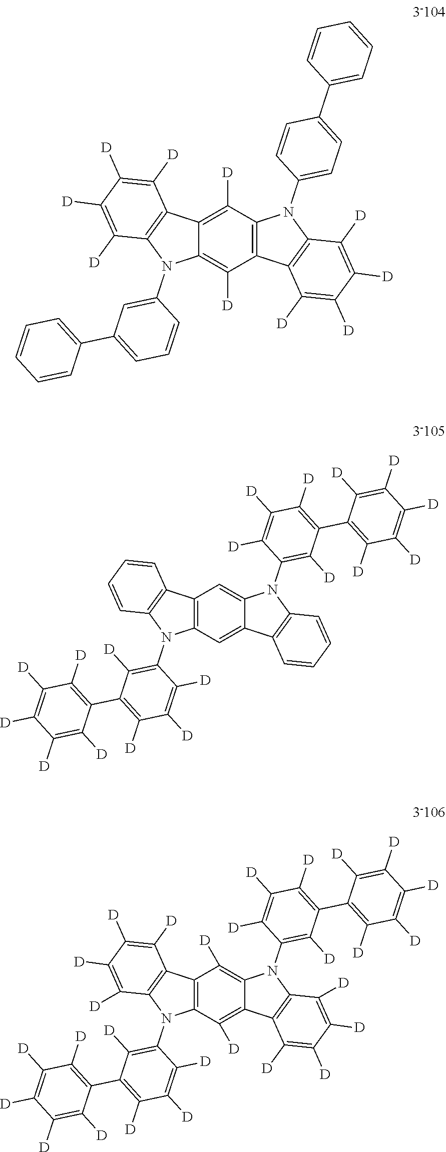

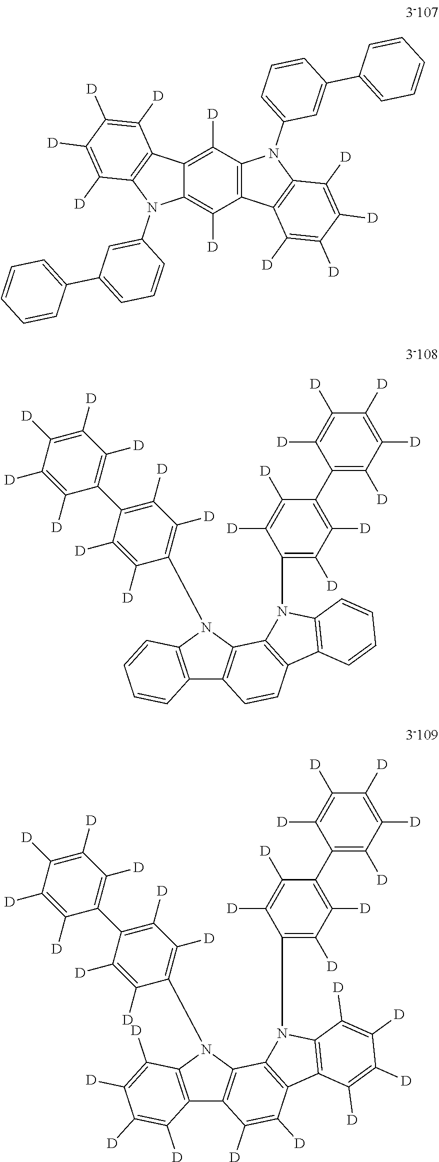

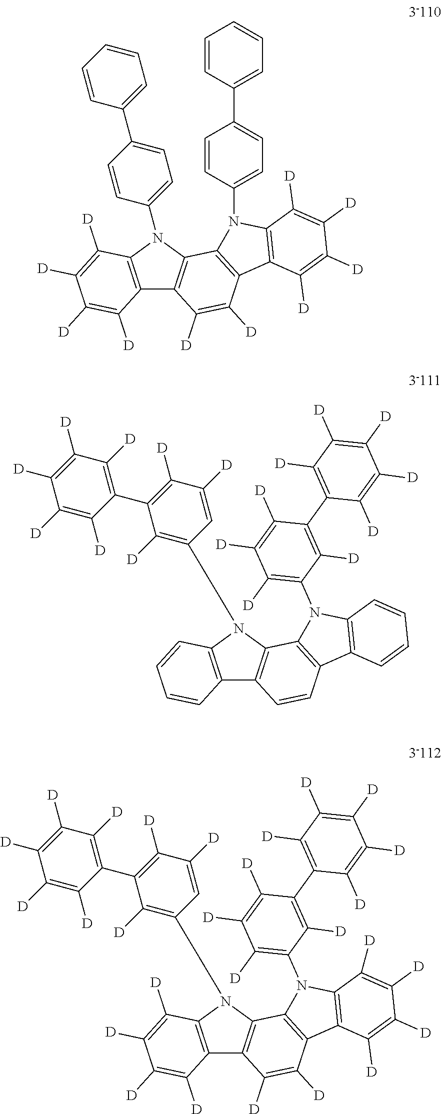

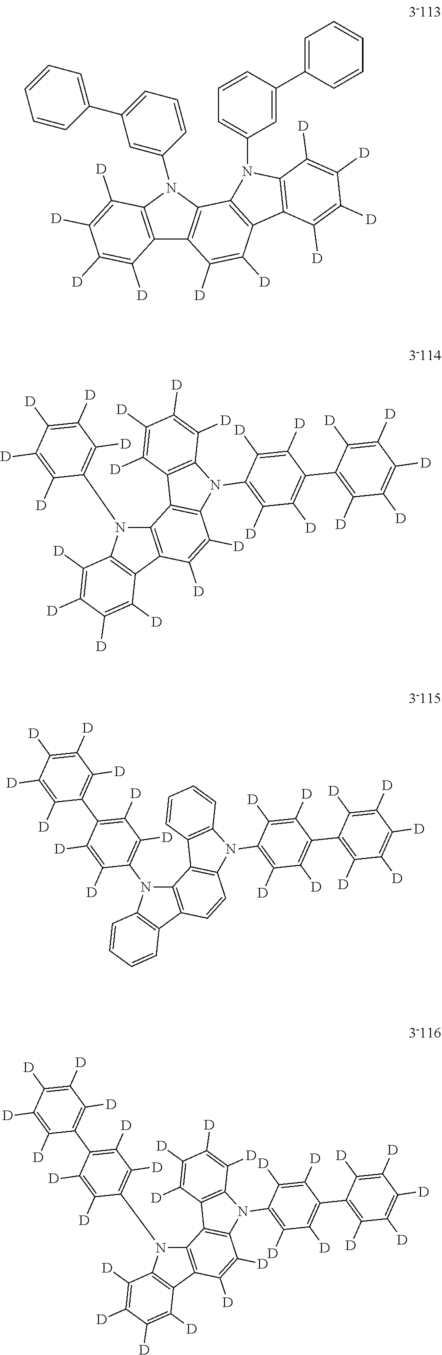

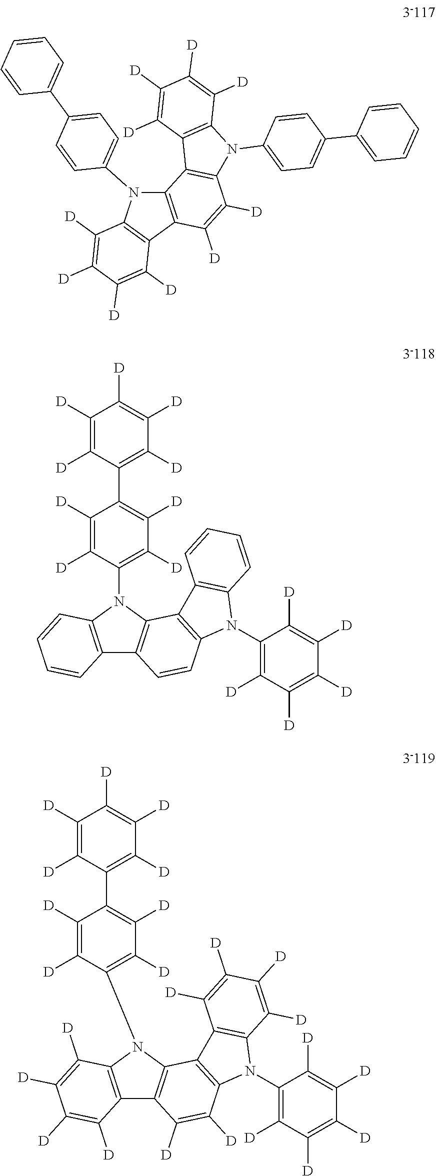 [SMILES: [2H]c1c([2H])c([2H])c2c(c1[2H])c1c(c([2H])c([2H])c3c4c([2H])c([2H])c([2H])c([2H])c4n(-c4ccc(-c5ccccc5)cc4)c31)n2-c1ccc(-c2ccccc2)cc1 ; [2H]c1c([2H])c([2H])c(-c2c([2H])c([2H])c(-n3c4ccccc4c4ccc5c(c6ccccc6n5-c5c([2H])c([2H])c([2H])c([2H])c5[2H])c43)c([2H])c2[2H])c([2H])c1[2H] ; [2H]c1c([2H])c([2H])c(-c2c([2H])c([2H])c(-n3c4c([2H])c([2H])c([2H])c([2H])c4c4c([2H])c([2H])c5c(c6c([2H])c([2H])c([2H])c([2H])c6n5-c5c([2H])c([2H])c([2H])c([2H])c5[2H])c43)c([2H])c2[2H])c([2H])c1[2H]]

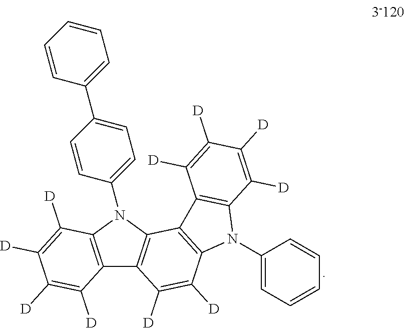 [SMILES: [2H]c1c([2H])c([2H])c2c(c1[2H])c1c(c([2H])c([2H])c3c4c([2H])c([2H])c([2H])c([2H])c4n(-c4ccc(-c5ccccc5)cc4)c31)n2-c1ccccc1]

14. The organic light emitting device of claim 9, further comprising one, two or more layers selected from the group consisting of a light emitting layer, a hole injection layer, a hole transport layer, a hole transport auxiliary layer, an electron injection layer, an electron transport layer, an electron blocking layer and a hole blocking layer.

15. A composition for an organic material layer, the composition comprising:
the heterocyclic compound of claim 1; and
a heterocyclic compound represented by the following Chemical Formula 2 or 3: 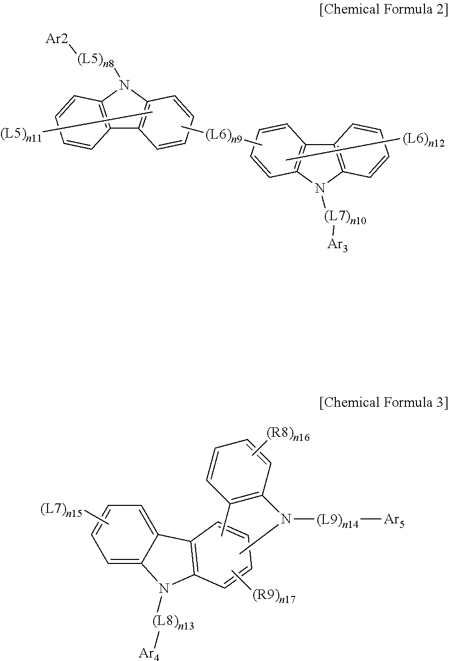
in Chemical Formulae 2 and 3,
L5 to L9 are the same as or different from each other, and each independently a single bond; a substituted or unsubstituted C6 to C60 arylene group; or a substituted or unsubstituted C2 to C60 heteroarylene group;
Ar2 to Ar5 are the same as or different from each other, and each independently a substituted or unsubstituted C6 to C60 aryl group; or a substituted or unsubstituted C2 to C60 heteroaryl group;
R5 to R9 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102 and —SiR101R102R103, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group;
n8 to n10, n13 and n14 are the same as or different from each other, and each independently an integer of 0 to 3;
n11 and n12 are an integer of 0 to 7;
n15 and n16 are an integer of 0 to 4; and
n17 is an integer of 0 to 2.

16. The composition of claim 15, wherein, in the composition, the heterocyclic compound represented by Chemical Formula 1 and the heterocyclic compound represented by Chemical Formula 2 or 3 have a weight ratio of 1:10 to 10:1.
